(19) United States
(12) Patent Application Publication
(10) Pub. No.: US 2026/0191199 A1
(43) Pub. Date: Jul. 9, 2026

(54) AMIDE COMPOUND AND HARMFUL ARTHROPOD CONTROL COMPOSITION CONTAINING SAME

(71) Applicant: SUMITOMO CHEMICAL COMPANY, LIMITED, Tokyo (JP)

(72) Inventors: Yasushi KATAGIRI, Takarazuka-shi (JP); Natsumi SATO, Takarazuka-shi (JP); Yusuke NAMBA, Takarazuka-shi (JP)

(73) Assignee: SUMITOMO CHEMICAL COMPANY, LIMITED, Tokyo (JP)

(21) Appl. No.: 19/131,713

(22) PCT Filed: Dec. 1, 2023
(86) PCT No.: PCT/JP2023/043063

§ 371 Date: May 21, 2025

(30) Foreign Application Priority Data
Dec. 5, 2022 (JP) 2022-194053

(51) Int. Cl. A01N 43/80; A01P 7/00; C07D 413/06

(52) U.S. Cl. CPC A01N 43/80; A01P 7/00; C07D 413/06

(57) ABSTRACT

The present invention provides a compound having an excellent control efficacy against harmful arthropods.

A compound represented by formula (I) [wherein R¹ and R² are identical to or different from each other, and each represent a C1-C6 chain hydrocarbon group or the like; p represents 0, 1, 2, 3, 4, 5, or 6; R³, R⁴, R⁵, R⁶, R⁷, and R⁸ are identical to or different from each other, and each represent a C1-C6 alkyl group optionally having one or more halogen atoms or the like; when p is 2, 3, 4, 5, or 6, R³ and R⁴ of 2, 3, 4, 5, or 6 are each independently identical to or different from each other; R⁹ represents a C1-C3 chain hydrocarbon group or the like; R¹⁰ and R¹¹ are identical to or different from each other, and each represent a C1-C4 alkyl group optionally having one or more halogen atoms or the like; R¹², R¹³, R¹⁴, R¹⁵, and R¹⁶ are identical to or different from each other, and each represent a C1-C4 chain hydrocarbon group or the like; and X represents an oxygen atom or the like] or a salt thereof has an excellent control efficacy against harmful arthropods.

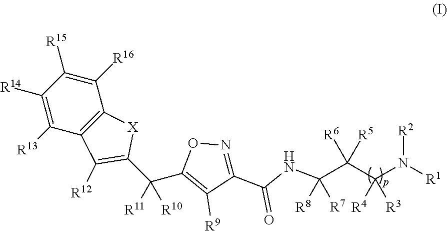

### TECHNICAL FIELD

This patent application claims the priority to and the benefit of Japanese Patent Application No. 2022-194053 (filed on Dec. 5, 2022), the entire contents of which are incorporated herein by reference.

The present invention relates to an amide compound and a harmful arthropod control composition containing the same.

### BACKGROUND ART

Various compounds have been investigated so far for controlling harmful arthropods. For example, Patent Document 1 describes that certain compounds have a harmful organism control effect.

### CITATION LIST

### Patent Document

Patent Document 1: WO 2014/119696 A

### SUMMARY OF THE INVENTION

### Problems to be Solved by the Invention

An object of the present invention is to provide a compound having an excellent control efficacy against harmful arthropods.

### Means for Solving the Problems

The present invention is as follows.
[1] A compound represented by formula (I)

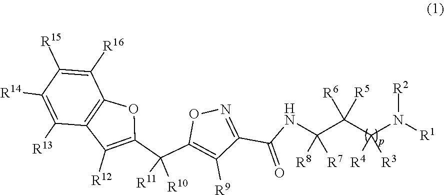

[wherein:
R¹ and R² are identical to or different from each other, and each represent a C1-C6 chain hydrocarbon group optionally having one or more substituents selected from Group A¹, a C1-C3 alkoxy group optionally having one or more halogen atoms, a benzyl group optionally having one or more substituents selected from Group B, a C1-C3 alkylcarbonyl group optionally having one or more halogen atoms, a C1-C3 alkoxycarbonyl group optionally having one or more halogen atoms, a C1-C3 alkylsulfonyl group optionally having one or more halogen atoms, or a hydrogen atom;
R¹ and R² may be combined to form —(CR¹⁷R¹⁸)—(CR¹⁹R²⁰)_{b}—(CR²⁵R²⁶)—, or —(CR²⁷R²⁸)—(CR²⁹R³⁰)—Y—(CR³¹R³²)—(CR³³R³⁴)—;
b represents 0, 1, 2, or 3;
Y represents an oxygen atom, a sulfur atom, or NR³⁵;
R¹⁷, R¹⁸, R¹⁹, R²⁰, R²⁵, R²⁶, R²⁷, R²⁸, R²⁹, R³⁰, R³¹, R³², R³³, R³⁴, and R³⁵ are identical to or different from each other, and each represent a C1-C4 alkyl group optionally having one or more halogen atoms, or a hydrogen atom;
when b is 2 or 3, R¹⁹ and R²⁰ of 2 or 3 may be each independently identical to or different from each other;
p represents 0, 1, 2, 3, 4, 5, or 6;
R³, R⁴, R⁵, R⁶, R⁷, and R⁸ are identical to or different from each other, and each represent a C1-C6 alkyl group optionally having one or more halogen atoms, or a hydrogen atom;
R³ and R⁴ may be combined to form —(CR³⁶R³⁷)—(CR³⁸R³⁹)ₑ—(CR⁴⁰R⁴¹)—, or —(CR⁴²R⁴³)ₕ—Z1 (CR⁴⁴R⁴⁵)ᵢ—;
R⁵ and R⁶ may be combined to form —(CR⁴⁶R⁴⁷)—(CR⁴⁸R⁴⁹)_{f}—(CR⁵⁰R⁵¹)—, or —(CR⁵²R⁵³)ⱼ—Z²—(CR⁵⁴R⁵⁵)ₖ—;
R⁷ and R⁸ may be combined to form —(CR⁵⁶R⁵⁷)—(CR⁵⁸R⁵⁹)_{g}—(CR⁶⁰R⁶¹)—, or —(CR⁶²R⁶³)ₘ—Z³—(CR⁶⁴R⁶⁵)ₙ—;
when p is 2, 3, 4, 5, or 6, R³ and R⁴ of 2, 3, 4, 5, or 6 may be each independently identical to or different from each other;
e, f, and g each independently represent 0, 1, 2, or 3;
h, i, j, k, m, and n each independently represent 1 or 2;
Z¹, Z², and Z³ are identical to or different from each other, and each represent an oxygen atom, a sulfur atom, or NR⁷⁰;
R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, and R⁷⁰ are identical to or different from each other, and each represent a C1-C4 alkyl group optionally having one or more halogen atoms, or a hydrogen atom;
when e, f, or g is 2 or 3, R³⁸, R³⁹, R⁴⁸, R⁴⁹, R⁵⁸, and R⁵⁹ of 2 or 3 may be each independently identical to or different from each other;
when h, i, j, k, m, or n is 2, R⁴², R⁴³, R⁴⁴, R⁴⁵, R⁵², R⁵³, R⁵⁴, R⁵⁵, R⁶², R⁶³, R⁶⁴, and R⁶⁵ of 2 may be each independently identical to or different from each other;
a plurality of R⁷⁰ (s) may be each independently identical to or different from each other;
R⁹ represents a C1-C3 chain hydrocarbon group optionally having one or more substituents selected from Group D, a cyano group, a formyl group, a carboxy group, a (C1-C4 alkoxy) carbonyl group wherein the C1-C4 alkoxy group optionally has one or more halogen atoms, a carbamoyl group, a halogen atom, or a hydrogen atom;
R¹⁰ and R¹¹ are identical to or different from each other, and each represent a C1-C4 alkyl group optionally having one or more halogen atoms, a halogen atom, or a hydrogen atom;
R¹², R¹³, R¹⁴, R¹⁵, and R¹⁶ are identical to or different from each other, and each represent a C1-C4 chain hydrocarbon group optionally having one or more substituents selected from Group E, a C1-C4 alkoxy group optionally having one or more substituents selected from Group E, a C3-C6 alicyclic hydrocarbon group optionally having one or more substituents selected from Group E, a phenyl group optionally having one or more substituents selected from Group B, a (C1-C4 alkyl) carbonyl group wherein the C1-C4 alkyl group optionally has one or more halogen atoms, a (C1-C4 alkoxy) carbonyl group wherein the C1-C4 alkoxy group optionally has one or more halogen atoms, a (C1-C4 alkyl) carbonyloxy group wherein the C1-C4 alkyl group optionally has one or more halogen atoms, a cyano group, a formyl group, a carboxy group, a nitro group, a hydroxy group, a halogen atom, or a hydrogen atom;
R¹³ and R¹⁴ may be combined to form —O—(CR⁷⁶R⁷⁷)—O—;
R¹⁴ and R¹⁵ may be combined to form —O—(CR⁷⁸R⁷⁹)—O—;
R¹⁵ and R¹⁶ may be combined to form —O—(CR⁸⁰R⁸¹)—O—;
R⁷⁶, R⁷⁷, R⁷⁸, R⁷⁹, R⁸⁰, and R⁸¹ are identical to or different from each other, and each represent a C1-C4 alkyl group optionally having one or more halogen atoms, a halogen atom, or a hydrogen atom;
X represents an oxygen atom, a sulfur atom, or NR⁷¹; and
R⁷¹ represents a C1-C4 alkyl group optionally having one or more halogen atoms, a C1-C4 alkoxycarbonyl group optionally having one or more halogen atoms, or a hydrogen atom;
Group A¹: a group consisting of a C3-C6 alicyclic hydrocarbon group optionally having one or more substituents selected from Group E, a C1-C4 alkoxy group optionally having one or more halogen atoms, a C1-C4 alkylcarbonyl group optionally having one or more halogen atoms, a C1-C4 alkoxycarbonyl group optionally having one or more halogen atoms, a C1-C4 alkylsulfonyl group optionally having one or more halogen atoms, a pyridyl group, a furyl group, a thienyl group, an isoxazolyl group, a pyridazinyl group, a pyrimidinyl group, a pyrazinyl group {the pyridyl group, the furyl group, the thienyl group, the isoxazolyl group, the pyridazinyl group, the pyrimidinyl group, and the pyrazinyl group may have one or more substituents selected from Group B}, a halogen atom, a hydroxy group, a cyano group, and CONR⁷²R⁷³;
R⁷² and R⁷³ are identical to or different from each other, and each represent a C1-C4 alkyl group optionally having one or more halogen atoms or a hydrogen atom;
Group B: a group consisting of a C1-C4 alkyl group optionally having one or more halogen atoms, a C1-C4 alkoxy group optionally having one or more halogen atoms, a C1-C4 alkylthio group optionally having one or more halogen atoms, a C1-C4 alkylsulfinyl group optionally having one or more halogen atoms, a C1-C4 alkylsulfonyl group optionally having one or more halogen atoms, a (C1-C4 alkoxy) carbonyl group wherein the C1-C4 alkoxy group optionally has one or more halogen atoms, a phenyl group optionally having one or more halogen atoms, a phenoxy group optionally having one or more halogen atoms, a cyano group, a nitro group, a carboxy group, a hydroxy group, a halogen atom, and CONR⁷⁴R⁷⁵;
R⁷⁴ and R⁷⁵ are identical to or different from each other, and each represent a C1-C4 alkyl group optionally having one or more halogen atoms or a hydrogen atom;
Group D: a group consisting of a hydroxy group and a halogen atom;
Group E: a group consisting of a C1-C4 alkoxy group optionally having one or more halogen atoms, a cyano group, a hydroxy group, and a halogen atom] (hereinafter, referred to as “Compound Z of the present invention) or a salt thereof (hereinafter, the Compound Z of the present invention or a salt thereof is referred to as “Compound W of the present invention”).
[2] The compound according to [1] Compound N of the present invention, wherein
R¹ and R² are identical to or different from each other, and each represent a C1-C6 chain hydrocarbon group optionally having one or more substituents selected from Group A, a C1-C3 alkoxy group optionally having one or more halogen atoms, a benzyl group optionally having one or more substituents selected from Group B, a C1-C3 alkylcarbonyl group optionally having one or more halogen atoms, a C1-C3 alkoxycarbonyl group optionally having one or more halogen atoms, a C1-C3 alkylsulfonyl group optionally having one or more halogen atoms, or a hydrogen atom; or
R¹ and R² are combined to form —(CR¹⁷R¹⁸)—(CR¹⁹R²⁰)_{b}—(CR²⁵R²⁶)—, or —(CR²⁷R²⁸)—(CR²⁹R³⁰)—Y—(CR³¹R³²)—(CR³³R³⁴)—, (hereinafter, referred to as “Compound N of the present invention) or a salt thereof (hereinafter, the Compound N of the present invention or a salt thereof is referred to as “Compound of the present invention”):
Group A: a group consisting of a C3-C6 alicyclic hydrocarbon group optionally having one or more substituents selected from Group E, a C1-C4 alkoxy group optionally having one or more halogen atoms, a C1-C4 alkylcarbonyl group optionally having one or more halogen atoms, a C1-C4 alkoxycarbonyl group optionally having one or more halogen atoms, a C1-C4 alkylsulfonyl group optionally having one or more halogen atoms, a pyridyl group, a furyl group, a thienyl group, a pyridazinyl group, a pyrimidinyl group, a pyrazinyl group {the pyridyl group, the furyl group, the thienyl group, the pyridazinyl group, the pyrimidinyl group, and the pyrazinyl group may have one or more substituents selected from Group B}, a halogen atom, a hydroxy group, a cyano group, and CONR⁷²R⁷³.
[3] The compound or a salt thereof according to [1], wherein
R¹ and R² are identical to or different from each other, and each represent a C1-C6 chain hydrocarbon group optionally having one or more substituents selected from Group A¹, a C1-C3 alkoxy group optionally having one or more halogen atoms, a benzyl group optionally having one or more substituents selected from Group F, or a hydrogen atom; or
R¹ and R² are combined to form —(CR¹⁷R¹⁸)—(CR¹⁹R²⁰)_{b}—(CR²⁵R²⁶)—, or —(CR²⁷R²⁸)—(CR²⁹R³⁰)—Y—(CR³¹R³²)—(CR³³R³⁴)—:
Group F: a group consisting of a C1-C4 alkyl group optionally having one or more halogen atoms, a C1-C4 alkoxy group optionally having one or more halogen atoms, a (C1-C4 alkoxy) carbonyl group wherein the C1-C4 alkoxy group optionally has one or more halogen atoms, a cyano group, a nitro group, a carboxy group, a hydroxy group, and a halogen atom,
[4] The compound or a salt thereof according to [2], wherein
R¹ and R² are identical to or different from each other, and each represent a C1-C6 chain hydrocarbon group optionally having one or more substituents selected from Group A, a C1-C3 alkoxy group optionally having one or more halogen atoms, a benzyl group optionally having one or more substituents selected from Group F, or a hydrogen atom; or
R¹ and R² are combined to form —(CR¹⁷R¹⁸)—(CR¹⁹R²⁰)_{b}—(CR²⁵R²⁶)—, or —(CR²⁷R²⁸)—(CR²⁹R³⁰)—Y—(CR³¹R³²)—(CR³³R³⁴)—:
Group F: a group consisting of a C1-C4 alkyl group optionally having one or more halogen atoms, a C1-C4 alkoxy group optionally having one or more halogen atoms, a (C1-C4 alkoxy) carbonyl group wherein the C1-C4 alkoxy group optionally has one or more halogen atoms, a cyano group, a nitro group, a carboxy group, a hydroxy group, and a halogen atom.
[5] The compound or a salt thereof according to any one of [1] to [4], wherein
R¹², R¹³, R¹⁴, R¹⁵, and R¹⁶ are identical to or different from each other, and each represent a C1-C4 chain hydrocarbon group optionally having one or more substituents selected from Group E, a C1-C4 alkoxy group optionally having one or more substituents selected from Group E, a C3-C6 alicyclic hydrocarbon group optionally having one or more substituents selected from Group E, a cyano group, a formyl group, a carboxy group, a nitro group, a hydroxy group, a halogen atom, or a hydrogen atom.
[6] The compound or a salt thereof according to any one of [1] to [5], wherein
X represents an oxygen atom or a sulfur atom.
[7] The compound or a salt thereof according to any one of [1] to [6], wherein
R⁹ represents a C1-C3 chain hydrocarbon group optionally having one or more substituents selected from Group D or a hydrogen atom.
[8] The compound or a salt thereof according to any one of [1] to [7], wherein
R¹⁰ and R¹¹ each represent a hydrogen atom.
[9] The compound or a salt thereof according to any one of [1] to [8], wherein
p represents 0 or 1.
[10] The compound or a salt thereof according to any one of [1] to [8], wherein
p represents 0.
[11] A composition for controlling a harmful arthropod comprising the compound or a salt thereof according to any one of [1] to [10], and an inert carrier.
[12] A composition comprising one or more ingredients selected from the group consisting of Group (a), Group (b), Group (c), and Group (d), and the compound or a salt thereof according to any one of [1] to [10]:
Group (a): a group consisting of insecticidal active ingredients, miticidal active ingredients, and nematicidal active ingredients;
Group (b): fungicidal active ingredients;
Group (c): plant growth modulating ingredients; and
Group (d): repellent ingredients.
[13] A method for controlling a harmful arthropod, which comprises applying an effective amount of the compound or a salt thereof according to any one of [1] to [10], or an effective amount of the composition according to [12] to a harmful arthropod or a habitat where the harmful arthropod lives.
[14] A seed or vegetative reproductive organ carrying an effective amount of the compound or a salt thereof according to any one of [1] to [10], or an effective amount of the composition according to [12].

### Effect of the Invention

The present invention can control harmful arthropods.

### MODE FOR CARRYING OUT THE INVENTION

The substituent in the present invention will be described.

The halogen atom means a fluorine atom, a chlorine atom, a bromine atom, or an iodine atom.

When the substituent has two or more halogen atoms or substituents, the halogen atoms or substituents may be identical to or different from each other.

The notation of “CX—CY” in the present description means that the number of carbon atoms is X to Y. For example, the notation of “C1-C6” means that the number of carbon atoms is 1 to 6.

The chain hydrocarbon group represents an alkyl group, an alkenyl group, or an alkynyl group.

Examples of the alkyl group include a methyl group, an ethyl group, a propyl group, an isopropyl group, a 1,1-dimethylpropyl group, a 1,2-dimethylpropyl group, a 1-ethylpropyl group, a butyl group, a sec-butyl group, a tert-butyl group, a pentyl group, and a hexyl group.

Examples of the alkenyl group include a vinyl group, a 1-propenyl group, a 2-propenyl group, a 1-methyl-1-propenyl group, a 1-methyl-2-propenyl group, a 1,2-dimethyl-1-propenyl group, a 1-ethyl-2-propenyl group, a 3-butenyl group, a 4-pentenyl group, and a 5-hexenyl group. Examples of the alkynyl group include an ethynyl group, a 1-propynyl group, a 2-propynyl group, a 1-methyl-2-propynyl group, a 1,1-dimethyl-2-propynyl group, a 1-ethyl-2-propynyl group, a 2-butynyl group, a 4-pentynyl group, and a 5-hexynyl group.

Examples of the alkoxy group include a methoxy group, an ethoxy group, a propoxy group, an isopropoxy group, a butoxy group, a tert-butoxy group, a pentyloxy group, and a hexyloxy group.

Examples of the alkylcarbonyl group include an acetyl group, a propanoyl group, a 2-methylpropanoyl group, and a hexanoyl group.

Examples of the alkoxycarbonyl group include a methoxycarbonyl group, an ethoxycarbonyl group, an isopropoxycarbonyl group, and a pentyloxycarbonyl group.

Examples of the alkylsulfonyl group include a methylsulfonyl group, an ethylsulfonyl group, a propylsulfonyl group, and an isopropylsulfonyl group.

The alicyclic hydrocarbon group represents a cycloalkyl group, a cycloalkenyl group, or a cycloalkynyl group.

Examples of the cycloalkyl group include a cyclopropyl group, a cyclobutyl group, a cyclopentyl group, and a cyclohexyl group.

Examples of the cycloalkenyl group include a cyclopropenyl group, a cyclobutenyl group, a cyclopentenyl group, and a cyclohexenyl group.

Examples of the cycloalkynyl group include a cyclohexynyl group.

The Compound W of the present invention may have one or more stereoisomers. Stereoisomers include enantiomers, diastereomers, geometric isomers, and the like. The Compound W of the present invention includes each stereoisomer and a stereoisomer mixture in any ratio.

The Compound Z of the present invention may form an acid addition salt. Examples of the acid forming the acid addition salt include inorganic acids such as hydrogen chloride, phosphoric acid, and sulfuric acid, and organic acids such as acetic acid, trifluoroacetic acid, benzoic acid, and p-toluenesulfonic acid. The acid addition salt is obtained, for example, by mixing the Compound Z of the present invention and an acid.

Examples of an embodiment of the Compound Z of the present invention include the following compounds.
[Embodiment Z1] The Compound Z of the present invention, wherein R¹ and R² are identical to or different from each other, and each represent a C1-C6 chain hydrocarbon group optionally having one or more substituents selected from the group A¹, a C1-C3 alkoxy group optionally having one or more halogen atoms, a benzyl group optionally having one or more substituents selected from the group B, or a hydrogen atom, or R¹ and R² are combined to form —(CH₂)—(CH₂)_{b}—(CH₂)—, or —(CH₂)—(CH₂)—Y—(CH₂)—(CH₂)—.
[Embodiment Z2] The Compound Z of the present invention, wherein R^{I} and R² are identical to or different from each other, and each represent a C1-C6 chain hydrocarbon group optionally having one or more substituents selected from the group A¹, a C1-C3 alkoxy group optionally having one or more halogen atoms, a benzyl group optionally having one or more substituents selected from a group B, or a hydrogen atom.
[Embodiment Z3] The compound according to Embodiment Z1, wherein R⁵ and R⁶ are identical to or different from each other, and each represent a C1-C6 alkyl group optionally having one or more halogen atoms or a hydrogen atom, or R⁵ and R⁶ are combined to form —(CH₂)—(CH₂)_{f}—(CH₂)— or —(CH₂)ⱼ—Z²—(CH₂)ₖ—.
[Embodiment Z4] The compound according to Embodiment Z1, wherein R⁵ and R⁶ are identical to or different from each other, and each represent a C1-C6 alkyl group or a hydrogen atom, or R⁵ and R⁶ are combined to form —(CH₂)—(CH₂)—(CH₂)—,
[Embodiment Z5] The compound according to Embodiment Z2, wherein R⁵ and R⁶ are identical to or different from each other, and each represent a C1-C6 alkyl group optionally having one or more halogen atoms or a hydrogen atom, or R⁵ and R⁶ are combined to form —(CH₂)—(CH₂)—(CH₂)— or —(CH₂)ⱼ—Z²—(CH₂)ₖ″.
[Embodiment Z6] The compound according to Embodiment 22, wherein R⁵ and R⁶ are identical to or different from each other, and each represent a C1-C6 alkyl group or a hydrogen atom, or R⁵ and R⁶ are combined to form —(CH₂)—(CH₂)_{f}—(CH₂)—.
[Embodiment Z7] The compound according to Embodiment Z3, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a formyl group, a carboxy group, a nitro group, a hydroxy group, a halogen atom, or a hydrogen atom, and R¹³ and R¹⁴ may be combined to form —O—(CH₂)—O—, and R¹⁴ and R¹⁵ may be combined to form —O—(CH₂)—O—, or, R¹⁵ and R¹⁶ may be combined to form —O—(CH₂)—O—.
[Embodiment Z8] The compound according to Embodiment Z4, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a formyl group, a carboxy group, a nitro group, a hydroxy group, a halogen atom, or a hydrogen atom, and R¹³ and R¹⁴ may be combined to form —O—(CH₂)—O—, and R¹⁴ and R¹⁵ may be combined to form —O—(CH₂)—O—, or, R¹⁵ and R¹⁶ may be combined to form —O—(CH₂)—O—.
[Embodiment Z9] The compound according to Embodiment Z5, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a formyl group, a carboxy group, a nitro group, a hydroxy group, a halogen atom, or a hydrogen atom, and R¹³ and R¹⁴ may be combined to form —O—(CH₂)—O—, and R¹⁴ and R¹⁵ may be combined to form —O—(CH₂)—O—, or, R¹⁵ and R¹⁶ may be combined to form —O—(CH₂)—O—,
[Embodiment Z10] The compound according to Embodiment Z6, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a formyl group, a carboxy group, a nitro group, a hydroxy group, a halogen atom, or a hydrogen atom, and R¹³ and R¹⁴ may be combined to form —O—(CH₂)—O—, and R¹⁴ and R¹⁵ may be combined to form —O—(CH₂)—O—, or, R¹⁵ and R¹⁶ may be combined to form —O—(CH₂)—O—.
[Embodiment Z11] The compound according to Embodiment Z3, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment Z12] The compound according to Embodiment Z4, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment Z13] The compound according to Embodiment Z5, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment Z14] The compound according to Embodiment 26, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment Z15] The compound according to Embodiment Z7, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}— (CH₂)— or —(CH₂)ₘ—Z³—(CH₂)ₙ—.
[Embodiment Z16] The compound according to Embodiment Z8, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH₂)— or —(CH₂)ₘ—Z³—(CH₂)ₙ—.
[Embodiment Z17] The compound according to Embodiment Z9, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH₂)— or —(CH₂)ₘ—Z³—(CH₂)ₙ—.
[Embodiment Z18] The compound according to Embodiment Z10, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH₂)— or —(CH₂)ₘ—Z³—(CH₂)ₙ—.
[Embodiment Z19] The compound according to Embodiment Z11, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)—(CH₂)— or —(CH₂)ₘ—Z³—(CH₂)ₙ—.
[Embodiment Z20] The compound according to Embodiment Z12, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH₂)— or —(CH₂)ₘ—Z³—(CH₂)ₙ—.
[Embodiment Z21] The compound according to Embodiment Z13, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH)— or —(CH₂)ₘ—Z³—(CH₂)ₙ—.
[Embodiment Z22] The compound according to Embodiment Z14, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH₂)— or —(CH₂)ₘ—Z³—(CH₂)ₙ—.
[Embodiment Z23] The compound according to Embodiment Z7, wherein R⁷ and R⁸ are identical to or different from each other, and each represent a C1-C6 alkyl group or a hydrogen atom.
[Embodiment Z24] The compound according to Embodiment Z8, wherein R⁷ and R⁸ are identical to or different from each other, and each represent a C1-C6 alkyl group or a hydrogen atom.
[Embodiment Z25] The compound according to Embodiment Z9, wherein R⁷ and R⁸ are identical to or different from each other, and each represent a C1-C6 alkyl group or a hydrogen atom.
[Embodiment Z26] The compound according to Embodiment Z10, wherein R⁷ and R⁸ are identical to or different from each other, and each represent a C1-C6 alkyl group or a hydrogen atom.
[Embodiment Z27] The compound according to Embodiment Z11, wherein R⁷ and R⁸ are identical to or different from each other, and each represent a C1-C6 alkyl group or a hydrogen atom.
[Embodiment Z28] The compound according to Embodiment Z12, wherein R⁷ and R⁸ are identical to or different from each other, and each represent a C1-C6 alkyl group or a hydrogen atom.
[Embodiment Z29] The compound according to Embodiment Z13, wherein R⁷ and R⁸ are identical to or different from each other, and each represent a C1-C6 alkyl group or a hydrogen atom.
[Embodiment Z30] The compound according to Embodiment Z14, wherein R⁷ and R⁸ are identical to or different from each other, and each represent a C1-C6 alkyl group or a hydrogen atom.
[Embodiment Z31] The compound according to Embodiment Z15, wherein R⁹ is a C1-C3 chain hydrocarbon group optionally having one or more substituents selected from the group D, a halogen atom, or a hydrogen atom,
[Embodiment Z32] The compound according to Embodiment Z16, wherein R⁹ is a C1-C3 chain hydrocarbon group optionally having one or more substituents selected from the group D, a halogen atom, or a hydrogen atom.
[Embodiment Z33] The compound according to Embodiment Z17, wherein R⁹ is a C1-C3 chain hydrocarbon group optionally having one or more substituents selected from the group D, a halogen atom, or a hydrogen atom,
[Embodiment Z34] The compound according to Embodiment Z18, wherein R⁹ is a C1-C3 chain hydrocarbon group optionally having one or more substituents selected from the group D, a halogen atom, or a hydrogen atom.
[Embodiment Z35] The compound according to Embodiment Z19, wherein R⁹ is a C1-C3 chain hydrocarbon group optionally having one or more substituents selected from the group D, a halogen atom, or a hydrogen atom,
[Embodiment Z36] The compound according to Embodiment Z20, wherein R⁹ is a C1-C3 chain hydrocarbon group optionally having one or more substituents selected from the group D, a halogen atom, or a hydrogen atom.
[Embodiment Z37] The compound according to Embodiment Z21, wherein R⁹ is a C1-C3 chain hydrocarbon group optionally having one or more substituents selected from the group D, a halogen atom, or a hydrogen atom.
[Embodiment Z38] The compound according to Embodiment Z22, wherein R⁹ is a C1-C3 chain hydrocarbon group optionally having one or more substituents selected from the group D, a halogen atom, or a hydrogen atom.
[Embodiment Z39] The compound according to Embodiment Z23, wherein R⁹ is a C1-C3 chain hydrocarbon group optionally having one or more substituents selected from the group D, a halogen atom, or a hydrogen atom.
[Embodiment Z40] The compound according to Embodiment Z24, wherein R⁹ is a C1-C3 chain hydrocarbon group optionally having one or more substituents selected from the group D, a halogen atom, or a hydrogen atom.
[Embodiment Z41] The compound according to Embodiment Z25, wherein R⁹ is a C1-C3 chain hydrocarbon group optionally having one or more substituents selected from the group D, a halogen atom, or a hydrogen atom.
[Embodiment Z42] The compound according to Embodiment Z26, wherein R⁹ is a C1-C3 chain hydrocarbon group optionally having one or more substituents selected from the group D, a halogen atom, or a hydrogen atom.
[Embodiment Z43] The compound according to Embodiment 227, wherein R⁹ is a C1-C3 chain hydrocarbon group optionally having one or more substituents selected from the group D, a halogen atom, or a hydrogen atom.
[Embodiment Z44] The compound according to Embodiment Z28, wherein R⁹ is a C1-C3 chain hydrocarbon group optionally having one or more substituents selected from the group D, a halogen atom, or a hydrogen atom,
[Embodiment Z45] The compound according to Embodiment Z29, wherein R⁹ is a C1-C3 chain hydrocarbon group optionally having one or more substituents selected from the group D, a halogen atom, or a hydrogen atom.
[Embodiment Z46] The compound according to Embodiment Z30, wherein R⁹ is a C1-C3 chain hydrocarbon group optionally having one or more substituents selected from the group D, a halogen atom, or a hydrogen atom.
[Embodiment Z47] The compound according to Embodiment Z15, wherein R⁹ is a hydrogen atom. [Embodiment Z48] The compound according to Embodiment Z16, wherein R⁹ is a hydrogen atom.
[Embodiment Z49] The compound according to Embodiment Z17, wherein R⁹ is a hydrogen atom,
[Embodiment Z50] The compound according to Embodiment Z18, wherein R⁹ is a hydrogen atom,
[Embodiment Z51] The compound according to Embodiment Z19, wherein R⁹ is a hydrogen atom.
[Embodiment Z52] The compound according to Embodiment Z20, wherein R⁹ is a hydrogen atom.
[Embodiment Z53] The compound according to Embodiment Z21, wherein R⁹ is a hydrogen atom.
[Embodiment Z54] The compound according to Embodiment Z22, wherein R⁹ is a hydrogen atom,
[Embodiment Z55] The compound according to Embodiment Z23, wherein R⁹ is a hydrogen atom.
[Embodiment Z56] The compound according to Embodiment 224, wherein R⁹ is a hydrogen atom.
[Embodiment Z57] The compound according to Embodiment Z25, wherein R⁹ is a hydrogen atom.
[Embodiment Z58] The compound according to Embodiment Z26, wherein R⁹ is a hydrogen atom,
[Embodiment Z59] The compound according to Embodiment Z27, wherein R⁹ is a hydrogen atom.
[Embodiment Z60] The compound according to Embodiment Z28, wherein R⁹ is a hydrogen atom.
[Embodiment Z61] The compound according to Embodiment Z29, wherein R⁹ is a hydrogen atom.
[Embodiment Z62] The compound according to Embodiment Z30, wherein R⁹ is a hydrogen atom,
[Embodiment Z63] The compound according to any one of Embodiment Z1 to Embodiment Z62 or the Compound Z of the present invention, wherein p is 0 or 1, and R³ and R⁴ are a hydrogen atom.
[Embodiment Z64] The compound according to any one of Embodiment Z1 to Embodiment Z62 or the Compound Z of the present invention, wherein p is 0.
[Embodiment Z65] The compound according to any one of Embodiment Z1 to Embodiment Z62 or the Compound Z of the present invention, wherein R¹⁰ and R¹¹ are a hydrogen atom.
[Embodiment Z66] The compound according to any one of Embodiment Z1 to Embodiment Z62 or the Compound Z of the present invention, wherein R¹⁰ and R¹¹ are a hydrogen atom, p is 0 or 1, and R³ and R⁴ are a hydrogen atom.
[Embodiment Z67] The compound according to any one of Embodiment Z1 to Embodiment Z62 or the Compound Z of the present invention, wherein R¹⁰ and R¹¹ are a hydrogen atom, and p is 0.
[Embodiment Z68] The compound according to any one of Embodiment Z1 to Embodiment Z62 or the Compound Z of the present invention, wherein R¹⁰ and R¹¹ are a hydrogen atom, and X is an oxygen atom or a sulfur atom.
[Embodiment Z69] The compound according to any one of Embodiment Z1 to Embodiment Z62 or the Compound Z of the present invention, wherein R¹⁰ and R¹¹ are a hydrogen atom, X is an oxygen atom or a sulfur atom, p is 0 or 1, and R³ and R⁴ are a hydrogen atom.
[Embodiment Z70] The compound according to any one of Embodiment Z1 to Embodiment Z62 or the Compound Z of the present invention, wherein R¹⁰ and R¹¹ are a hydrogen atom, X is an oxygen atom or a sulfur atom, and p is 0.
[Embodiment Z71] The compound according to any one of Embodiment Z1 to Embodiment Z62 or the Compound Z of the present invention, wherein R¹⁰ and R¹¹ are a hydrogen atom, and X is an oxygen atom.
[Embodiment Z72] The compound according to any one of Embodiment Z1 to Embodiment Z62 or the Compound Z of the present invention, wherein R¹⁰ and R¹¹ are a hydrogen atom, X is an oxygen atom, p is 0 or 1, and R³ and R⁴ are a hydrogen atom.
[Embodiment Z73] The compound according to any one of Embodiment Z1 to Embodiment Z62 or the Compound Z of the present invention, wherein R¹⁰ and R¹¹ are a hydrogen atom, X is an oxygen atom, and p is 0.
[Embodiment Z74] The compound according to any one of Embodiment Z1 to Embodiment Z62 or the Compound Z of the present invention, wherein R¹⁰ and R¹¹ are a hydrogen atom, and X is a sulfur atom.
[Embodiment Z75] The compound according to any one of Embodiment Z1 to Embodiment Z62 or the Compound Z of the present invention, wherein R¹⁰ and R¹¹ are a hydrogen atom, X is a sulfur atom, p is 0 or 1, and R³ and R⁴ are a hydrogen atom.
[Embodiment Z76] The compound according to any one of Embodiment Z1 to Embodiment Z62 or the Compound Z of the present invention, wherein R¹⁰ and R¹¹ are a hydrogen atom, X is a sulfur atom, and p is 0.
[Embodiment Z77] The compound according to any one of Embodiment Z1 to Embodiment Z62 or the Compound Z of the present invention, wherein R¹⁰ and R¹¹ are a hydrogen atom, and R¹² and R¹⁶ are a hydrogen atom.
[Embodiment Z78] The compound according to any one of Embodiment Z1 to Embodiment Z62 or the Compound Z of the present invention, wherein R¹⁰ and R¹¹ are a hydrogen atom, R¹² and R¹⁶ are a hydrogen atom, p is 0 or 1, and R³ and R⁴ are a hydrogen atom.
[Embodiment Z79] The compound according to any one of Embodiment Z1 to Embodiment Z62 or the Compound Z of the present invention, wherein R¹⁰ and R¹¹ are a hydrogen atom, R¹² and R¹⁶ are a hydrogen atom, and p is 0.
[Embodiment Z80] The compound according to any one of Embodiment Z1 to Embodiment Z62 or the Compound Z of the present invention, wherein R¹⁰ and R¹¹ are a hydrogen atom, R¹² and R¹⁶ are a hydrogen atom, and X is an oxygen atom or a sulfur atom.
[Embodiment Z81] The compound according to any one of Embodiment Z1 to Embodiment Z62 or the Compound Z of the present invention, wherein R¹⁰ and R¹¹ are a hydrogen atom, R¹² and R¹⁶ are a hydrogen atom, X is an oxygen atom or a sulfur atom, p is 0 or 1, and R³ and R⁴ are a hydrogen atom.
[Embodiment Z82] The compound according to any one of Embodiment Z1 to Embodiment Z62 or the Compound Z of the present invention, wherein R¹⁰ and R¹¹ are a hydrogen atom, R¹² and R¹⁶ are a hydrogen atom, X is an oxygen atom or a sulfur atom, and p is 0.
[Embodiment Z83] The compound according to any one of Embodiment Z1 to Embodiment Z62 or the Compound Z of the present invention, wherein R¹⁰ and R¹¹ are a hydrogen atom, R¹² and R¹⁶ are a hydrogen atom, and X is an oxygen atom.
[Embodiment Z84] The compound according to any one of Embodiment Z1 to Embodiment Z62 or the Compound Z of the present invention, wherein R¹⁰ and R¹¹ are a hydrogen atom, R¹² and R¹⁶ are a hydrogen atom, X is an oxygen atom, p is 0 or 1, and R³ and R⁴ are a hydrogen atom.
[Embodiment Z85] The compound according to any one of Embodiment Z1 to Embodiment Z62 or the Compound Z of the present invention, wherein R¹⁰ and R¹¹ are a hydrogen atom, R¹² and R¹⁶ are a hydrogen atom, X is an oxygen atom, and p is 0.
[Embodiment Z86] The compound according to any one of Embodiment Z1 to Embodiment Z62 or the Compound Z of the present invention, wherein R¹⁰ and R¹¹ are a hydrogen atom, R¹² and R¹⁶ are a hydrogen atom, and X is a sulfur atom,
[Embodiment Z87] The compound according to any one of Embodiment Z1 to Embodiment Z62 or the Compound Z of the present invention, wherein R¹⁰ and R¹¹ are a hydrogen atom, R¹² and R¹⁶ are a hydrogen atom, X is a sulfur atom, p is 0 or 1, and R³ and R⁴ are a hydrogen atom.
[Embodiment Z88] The compound according to any one of Embodiment Z1 to Embodiment Z62 or the Compound Z of the present invention, wherein R¹⁰ and R¹¹ are a hydrogen atom, R¹² and R¹⁶ are a hydrogen atom, X is a sulfur atom, and p is 0,
[Embodiment ZA1] The Compound Z of the present invention, wherein R¹⁰ and R¹¹ are a hydrogen atom,
[Embodiment ZA2] The Compound Z of the present invention, wherein R¹² and R¹⁶ are a hydrogen atom.
[Embodiment ZA3] The compound according to Embodiment ZA1, wherein R¹² and Ric are a hydrogen atom.
[Embodiment ZA3] The compound according to the Compound Z of the present invention, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment ZA4] The compound according to Embodiment ZA1, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment ZA5] The Compound Z of the present invention, wherein R¹² and R¹⁶ are a hydrogen atom, R¹³, R¹⁴, and R¹⁵ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment ZA6] The compound according to Embodiment ZA1, wherein R¹² and R¹⁶ are a hydrogen atom, R¹³, R¹⁴, and R¹⁵ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment ZA7] The Compound Z of the present invention, wherein R⁹ is a methyl group, a halogen atom, or a hydrogen atom.
[Embodiment ZA8] The compound according to Embodiment ZA1, wherein R⁹ is a methyl group, a halogen atom, or a hydrogen atom.
[Embodiment ZA9] The compound according to Embodiment ZA2, wherein R⁹ is a methyl group, a halogen atom, or a hydrogen atom.
[Embodiment ZA10] The compound according to Embodiment ZA3, wherein R⁹ is a methyl group, a halogen atom, or a hydrogen atom.
[Embodiment ZA11] The compound according to Embodiment ZA4, wherein R⁹ is a methyl group, a halogen atom, or a hydrogen atom,
[Embodiment ZA12] The compound according to Embodiment ZA5, wherein R⁹ is a methyl group, a halogen atom, or a hydrogen atom.
[Embodiment ZA13] The compound according to Embodiment ZA6, wherein R⁹ is a methyl group, a halogen atom, or a hydrogen atom.
[Embodiment ZA14] The Compound Z of the present invention, wherein R⁹ is a hydrogen atom.
[Embodiment ZA15] The compound according to Embodiment ZA1, wherein R⁹ is a hydrogen atom.
[Embodiment ZA16] The compound according to Embodiment ZA2, wherein R⁹ is a hydrogen atom.
[Embodiment ZA17] The compound according to Embodiment ZA3, wherein R⁹ is a hydrogen atom.
[Embodiment ZA18] The compound according to Embodiment ZA4, wherein R⁹ is a hydrogen atom.
[Embodiment ZA19] The compound according to Embodiment ZA5, wherein R⁹ is a hydrogen atom.
[Embodiment ZA20] The compound according to Embodiment ZA6, wherein R⁹ is a hydrogen atom.
[Embodiment ZA21] The compound according to any one of Embodiment ZA1 to Embodiment ZA20 or the Compound Z of the present invention, wherein X is an oxygen atom or a sulfur atom,
[Embodiment ZA22] The compound according to any one of Embodiment ZA1 to Embodiment ZA20 or the Compound Z of the present invention, wherein X is an oxygen atom.
[Embodiment ZA23] The compound according to any one of Embodiment ZA1 to Embodiment ZA20 or the Compound Z of the present invention, wherein X is a sulfur atom.

Examples of an embodiment of the Compound N of the present invention include the following compounds.
[Embodiment 1] The Compound N of the present invention, wherein R¹ and R² are identical to or different from each other, and each represent a C1-C6 chain hydrocarbon group optionally having one or more substituents selected from the group A, a C1-C3 alkoxy group optionally having one or more halogen atoms, a benzyl group optionally having one or more substituents selected from the group B, or a hydrogen atom, or R¹ and R² are combined to form —(CH₂)—(CH₂)_{b}—(CH₂)—, or —(CH₂)—(CH₂)—Y—(CH₂)—(CH₂)—.
[Embodiment 2] The Compound N of the present invention, wherein R¹ and R² are identical to or different from each other, and each represent a C1-C6 chain hydrocarbon group optionally having one or more substituents selected from the group A, a C1-C3 alkoxy group optionally having one or more halogen atoms, a benzyl group optionally having one or more substituents selected from a group B, or a hydrogen atom.
[Embodiment 3] The compound according to Embodiment 1, wherein R⁵ and R⁶ are identical to or different from each other, and each represent a C1-C6 alkyl group optionally having one or more halogen atoms or a hydrogen atom, or R⁵ and R⁶ are combined to form —(CH₂)—(CH₂)_{f}—(CH₂)— or —(CH₂)ⱼ—Z²—(CH₂)ₖ—.
[Embodiment 4] The compound according to Embodiment 1, wherein R⁵ and R⁶ are identical to or different from each other, and each represent a C1-C6 alkyl group or a hydrogen atom, or R⁵ and R⁶ are combined to form —(CH₂)—(CH₂)_{f}—(CH₂)—.
[Embodiment 5] The compound according to Embodiment 2, wherein R⁵ and R⁶ are identical to or different from each other, and each represent a C1-C6 alkyl group optionally having one or more halogen atoms or a hydrogen atom, or R⁵ and R⁶ are combined to form —(CH₂)—(CH₂)_{f}—(CH₂)— or —(CH₂)ⱼ—Z²—(CH₂)ₖ—.
[Embodiment 6] The compound according to Embodiment 2, wherein R⁵ and R⁶ are identical to or different from each other, and each represent a C1-C6 alkyl group or a hydrogen atom, or R⁵ and R⁶ are combined to form —(CH₂)—(CH₂)_{f}—(CH₂)—.
[Embodiment 7] The compound according to Embodiment 3, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a formyl group, a carboxy group, a nitro group, a hydroxy group, a halogen atom, or a hydrogen atom, and R¹³ and R¹⁴ may be combined to form —O—(CH₂)—O—, and R¹⁴ and R¹⁵ may be combined to form —O—(CH₂)—O—, or, R¹⁵ and R¹⁶ may be combined to form —O—(CH₂)—O—.
[Embodiment 8] The compound according to Embodiment 4, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a formyl group, a carboxy group, a nitro group, a hydroxy group, a halogen atom, or a hydrogen atom, and R¹³ and R¹⁴ may be combined to form —O—(CH₂)—O—, and R¹⁴ and R¹⁵ may be combined to form —O—(CH₂)—O—, or, R¹⁵ and R¹⁶ may be combined to form —O—(CH₂)—O—.
[Embodiment 9] The compound according to Embodiment 5, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a formyl group, a carboxy group, a nitro group, a hydroxy group, a halogen atom, or a hydrogen atom, and R¹³ and R¹⁴ may be combined to form —O—(CH₂)—O—, and R¹⁴ and R¹⁵ may be combined to form —O—(CH₂)—O—, or, R¹⁵ and R¹⁶ may be combined to form —O— (CH₂)—O—.
[Embodiment 10] The compound according to Embodiment 6, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a formyl group, a carboxy group, a nitro group, a hydroxy group, a halogen atom, or a hydrogen atom, and R¹³ and R¹⁴ may be combined to form —O—(CH₂)—O—, and R¹⁴ and R¹⁵ may be combined to form —O—(CH₂)—O—, or, R¹⁵ and R¹⁶ may be combined to form —O—(CH₂)—O—.
[Embodiment 11] The compound according to Embodiment 3, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment 12] The compound according to Embodiment 4, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment 13] The compound according to Embodiment 5, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment 14] The compound according to Embodiment 6, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment 15] The compound according to Embodiment 7, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH₂)— or —(CH₂)ₘ—Z³—(CH₂)ₙ—.
[Embodiment 16] The compound according to Embodiment 8, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH₂)— or —(CH₂)ₘ—Z³—(CH₂)ₙ—.
[Embodiment 17] The compound according to Embodiment 9, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH₂)— or —(CH₂)ₘ—Z³—(CH₂)ₙ—.
[Embodiment 18] The compound according to Embodiment 10, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH₂)— or —(CH₂)ₘ—Z³—(CH₂)ₙ—.
[Embodiment 19] The compound according to Embodiment 11, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH₂)— or —(CH₂)ₘ—Z³—(CH₂)ₙ—.
[Embodiment 20] The compound according to Embodiment 12, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH₂)— or —(CH₂)ₘ—Z³—(CH₂)ₙ—.
[Embodiment 21] The compound according to Embodiment 13, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH₂)— or —(CH₂)ₘ—Z³—(CH₂)ₙ—.
[Embodiment 22] The compound according to Embodiment 14, wherein R⁷ and R⁸ are identical to or different from each other, and each represent a C1-C6 alkyl group optionally having one or more halogen atoms or a hydrogen atom, or R⁷ and R⁸ are combined to form —(CH₂)—(CH₂)_{g}—(CH₂)— or —(CH₂)ₘ—Z³—(CH₂)ₙ—.
[Embodiment 23] The compound according to Embodiment 7, wherein R⁷ and R⁸ are identical to or different from each other, and each represent a C1-C6 alkyl group or a hydrogen atom.
[Embodiment 24] The compound according to Embodiment 8, wherein R⁷ and R⁸ are identical to or different from each other, and each represent a C1-C6 alkyl group or a hydrogen atom.
[Embodiment 25] The compound according to Embodiment 9, wherein R⁷ and R⁸ are identical to or different from each other, and each represent a C1-C6 alkyl group or a hydrogen atom.
[Embodiment 26] The compound according to Embodiment 10, wherein R⁷ and R⁸ are identical to or different from each other, and each represent a C1-C6 alkyl group or a hydrogen atom.
[Embodiment 27] The compound according to Embodiment 11, wherein R⁷ and R⁸ are identical to or different from each other, and each represent a C1-C6 alkyl group or a hydrogen atom.
[Embodiment 28] The compound according to Embodiment 12, wherein R⁷ and R⁸ are identical to or different from each other, and each represent a C1-C6 alkyl group or a hydrogen atom.
[Embodiment 29] The compound according to Embodiment 13, wherein R⁷ and R⁸ are identical to or different from each other, and each represent a C1-C6 alkyl group or a hydrogen atom.
[Embodiment 30] The compound according to Embodiment 14, wherein R⁷ and R⁸ are identical to or different from each other, and each represent a C1-C6 alkyl group or a hydrogen atom.
[Embodiment 31] The compound according to Embodiment 15, wherein R⁹ is a C1-C3 chain hydrocarbon group optionally having one or more substituents selected from the group D, a halogen atom, or a hydrogen atom,
[Embodiment 32] The compound according to Embodiment 16, wherein R⁹ is a C1-C3 chain hydrocarbon group optionally having one or more substituents selected from the group D, a halogen atom, or a hydrogen atom.
[Embodiment 33] The compound according to Embodiment 17, wherein R⁹ is a C1-C3 chain hydrocarbon group optionally having one or more substituents selected from the group D, a halogen atom, or a hydrogen atom,
[Embodiment 34] The compound according to Embodiment 18, wherein R⁹ is a C1-C3 chain hydrocarbon group optionally having one or more substituents selected from the group D, a halogen atom, or a hydrogen atom.
[Embodiment 35] The compound according to Embodiment 19, wherein R⁹ is a C1-C3 chain hydrocarbon group optionally having one or more substituents selected from the group D, a halogen atom, or a hydrogen atom,
[Embodiment 36] The compound according to Embodiment 20, wherein R⁹ is a C1-C3 chain hydrocarbon group optionally having one or more substituents selected from the group D, a halogen atom, or a hydrogen atom.
[Embodiment 37] The compound according to Embodiment 21, wherein R⁹ is a C1-C3 chain hydrocarbon group optionally having one or more substituents selected from the group D, a halogen atom, or a hydrogen atom,
[Embodiment 38] The compound according to Embodiment 22, wherein R⁹ is a C1-C3 chain hydrocarbon group optionally having one or more substituents selected from the group D, a halogen atom, or a hydrogen atom.
[Embodiment 39] The compound according to Embodiment 23, wherein R⁹ is a C1-C3 chain hydrocarbon group optionally having one or more substituents selected from the group D, a halogen atom, or a hydrogen atom,
[Embodiment 40] The compound according to Embodiment 24, wherein R⁹ is a C1-C3 chain hydrocarbon group optionally having one or more substituents selected from the group D, a halogen atom, or a hydrogen atom.
[Embodiment 41] The compound according to Embodiment 25, wherein R⁹ is a C1-C3 chain hydrocarbon group optionally having one or more substituents selected from the group D, a halogen atom, or a hydrogen atom,
[Embodiment 42] The compound according to Embodiment 26, wherein R⁹ is a C1-C3 chain hydrocarbon group optionally having one or more substituents selected from the group D, a halogen atom, or a hydrogen atom.
[Embodiment 43] The compound according to Embodiment 27, wherein R⁹ is a C1-C3 chain hydrocarbon group optionally having one or more substituents selected from the group D, a halogen atom, or a hydrogen atom,
[Embodiment 44] The compound according to Embodiment 28, wherein R⁹ is a C1-C3 chain hydrocarbon group optionally having one or more substituents selected from the group D, a halogen atom, or a hydrogen atom.
[Embodiment 45] The compound according to Embodiment 29, wherein R⁹ is a C1-C3 chain hydrocarbon group optionally having one or more substituents selected from the group D, a halogen atom, or a hydrogen atom.
[Embodiment 46] The compound according to Embodiment 30, wherein R⁹ is a C1-C3 chain hydrocarbon group optionally having one or more substituents selected from the group D, a halogen atom, or a hydrogen atom.
[Embodiment 47] The compound according to Embodiment 15, wherein R⁹ is a hydrogen atom,
[Embodiment 48] The compound according to Embodiment 16, wherein R⁹ is a hydrogen atom.
[Embodiment 49] The compound according to Embodiment 17, wherein R⁹ is a hydrogen atom.
[Embodiment 50] The compound according to Embodiment 18, wherein R⁹ is a hydrogen atom.
[Embodiment 51] The compound according to Embodiment 19, wherein R⁹ is a hydrogen atom, [Embodiment 52] The compound according to Embodiment 20, wherein R⁹ is a hydrogen atom.
[Embodiment 53] The compound according to Embodiment 21, wherein R⁹ is a hydrogen atom.
[Embodiment 54] The compound according to Embodiment 22, wherein R⁹ is a hydrogen atom.
[Embodiment 55] The compound according to Embodiment 23, wherein R⁹ is a hydrogen atom,
[Embodiment 56] The compound according to Embodiment 24, wherein R⁹ is a hydrogen atom.
[Embodiment 57] The compound according to Embodiment 25, wherein R⁹ is a hydrogen atom.
[Embodiment 58] The compound according to Embodiment 26, wherein R⁹ is a hydrogen atom.
[Embodiment 59] The compound according to Embodiment 27, wherein R⁹ is a hydrogen atom.
[Embodiment 60] The compound according to Embodiment 28, wherein R⁹ is a hydrogen atom.
[Embodiment 61] The compound according to Embodiment 29, wherein R⁹ is a hydrogen atom.
[Embodiment 62] The compound according to Embodiment 30, wherein R⁹ is a hydrogen atom.
[Embodiment 63] The compound according to any one of Embodiment 1 to Embodiment 62 or the Compound N of the present invention, wherein p is 0 or 1, and R³ and R⁴ are a hydrogen atom.
[Embodiment 64] The compound according to any one of Embodiment 1 to Embodiment 62 or the Compound N of the present invention, wherein p is 0.
[Embodiment 65] The compound according to any one of Embodiment 1 to Embodiment 62 or the Compound N of the present invention, wherein R¹⁰ and R¹¹ are a hydrogen atom.
[Embodiment 66] The compound according to any one of Embodiment 1 to Embodiment 62 or the Compound N of the present invention, wherein R¹⁰ and R¹¹ are a hydrogen atom, p is 0 or 1, and R³ and R⁴ are a hydrogen atom.
[Embodiment 67] The compound according to any one of Embodiment 1 to Embodiment 62 or the Compound N of the present invention, wherein R¹⁰ and R¹¹ are a hydrogen atom and p is 0.
[Embodiment 68] The compound according to any one of Embodiment 1 to Embodiment 62 or the Compound N of the present invention, wherein R¹⁰ and R¹¹ are a hydrogen atom, and X is an oxygen atom or a sulfur atom.
[Embodiment 69] The compound according to any one of Embodiment 1 to Embodiment 62 or the Compound N of the present invention, wherein R¹⁰ and R¹¹ are a hydrogen atom, X is an oxygen atom or a sulfur atom, p is 0 or 1, and R³ and R⁴ are a hydrogen atom.
[Embodiment 70] The compound according to any one of Embodiment 1 to Embodiment 62 or the Compound N of the present invention, wherein R¹⁰ and R¹¹ are a hydrogen atom, X is an oxygen atom or a sulfur atom, and p is 0.
[Embodiment 71] The compound according to any one of Embodiment 1 to Embodiment 62 or the Compound N of the present invention, wherein R¹⁰ and R¹¹ are a hydrogen atom, and X is an oxygen atom.
[Embodiment 72] The compound according to any one of Embodiment 1 to Embodiment 62 or the Compound N of the present invention, wherein R¹⁰ and R¹¹ are a hydrogen atom, X is an oxygen atom, p is 0 or 1, and R³ and R⁴ are a hydrogen atom.
[Embodiment 73] The compound according to any one of Embodiment 1 to Embodiment 62 or the Compound N of the present invention, wherein R¹⁰ and R¹¹ are a hydrogen atom, X is an oxygen atom, and p is 0.
[Embodiment 74] The compound according to any one of Embodiment 1 to Embodiment 62 or the Compound N of the present invention, wherein R¹⁰ and R¹¹ are a hydrogen atom, and X is a sulfur atom.
[Embodiment 75] The compound according to any one of Embodiment 1 to Embodiment 62 or the Compound N of the present invention, wherein R¹⁰ and R¹¹ are a hydrogen atom, X is a sulfur atom, p is 0 or 1, and R³ and R⁴ are a hydrogen atom.
[Embodiment 76] The compound according to any one of Embodiment 1 to Embodiment 62 or the Compound N of the present invention, wherein R¹⁰ and R¹¹ are a hydrogen atom, X is a sulfur atom, and p is 0.
[Embodiment 77] The compound according to any one of Embodiment 1 to Embodiment 62 or the Compound N of the present invention, wherein R¹⁰ and R¹¹ are a hydrogen atom, and R¹² and R¹⁶ are a hydrogen atom.
[Embodiment 78] The compound according to any one of Embodiment 1 to Embodiment 62 or the Compound N of the present invention, wherein R¹⁰ and R¹¹ are a hydrogen atom, R¹² and R¹⁶ are a hydrogen atom, p is 0 or 1, and R³ and R⁴ are a hydrogen atom.
[Embodiment 79] The compound according to any one of Embodiment 1 to Embodiment 62 or the Compound N of the present invention, wherein R¹⁰ and R¹¹ are a hydrogen atom, R¹² and R¹⁶ are a hydrogen atom, and p is 0.
[Embodiment 80] The compound according to any one of Embodiment 1 to Embodiment 62 or the Compound N of the present invention, wherein R¹⁰ and R¹¹ are a hydrogen atom, R¹² and R¹⁶ are a hydrogen atom, and X is an oxygen atom or a sulfur atom.
[Embodiment 81] The compound according to any one of Embodiment 1 to Embodiment 62 or the Compound N of the present invention, wherein R¹⁰ and R¹¹ are a hydrogen atom, R¹² and Rif are a hydrogen atom, X is an oxygen atom or a sulfur atom, p is 0 or 1, and R³ and R⁴ are a hydrogen atom.
[Embodiment 82] The compound according to any one of Embodiment 1 to Embodiment 62 or the Compound N of the present invention, wherein R¹⁰ and R¹¹ are a hydrogen atom, R¹² and R¹⁶ are a hydrogen atom, X is an oxygen atom or a sulfur atom, and p is 0.
[Embodiment 83] The compound according to any one of Embodiment 1 to Embodiment 62 or the Compound N of the present invention, wherein R¹⁰ and R¹¹ are a hydrogen atom, R¹² and R¹⁶ are a hydrogen atom, and X is an oxygen atom,
[Embodiment 84] The compound according to any one of Embodiment 1 to Embodiment 62 or the Compound N of the present invention, wherein R¹⁰ and R¹¹ are a hydrogen atom, R¹² and R¹⁶ are a hydrogen atom, X is an oxygen atom, p is 0 or 1, and R³ and R⁴ are a hydrogen atom.
[Embodiment 85] The compound according to any one of Embodiment 1 to Embodiment 62 or the Compound N of the present invention, wherein R¹⁰ and R¹¹ are a hydrogen atom, R¹² and R¹⁶ are a hydrogen atom, X is an oxygen atom, and p is 0.
[Embodiment 86] The compound according to any one of Embodiment 1 to Embodiment 62 or the Compound N of the present invention, wherein R¹⁰ and R¹¹ are a hydrogen atom, R¹² and R¹⁶ are a hydrogen atom, and X is a sulfur atom.
[Embodiment 87] The compound according to any one of Embodiment 1 to Embodiment 62 or the Compound N of the present invention, wherein R¹⁰ and R¹¹ are a hydrogen atom, R¹² and R¹⁶ are a hydrogen atom, X is a sulfur atom, p is 0 or 1, and R³ and R⁴ are a hydrogen atom.
[Embodiment 88] The compound according to any one of Embodiment 1 to Embodiment 62 or the Compound N of the present invention, wherein R¹⁰ and R¹¹ are a hydrogen atom, R¹² and R¹⁶ are a hydrogen atom, X is a sulfur atom, and p is 0.
[Embodiment A1] The Compound N of the present invention, wherein R¹⁰ and R¹¹ are a hydrogen atom,
[Embodiment A2] The Compound N of the present invention, wherein R¹² and R¹⁶ are a hydrogen atom.
[Embodiment A3] The compound according to Embodiment A1, wherein R¹² and R¹⁶ are a hydrogen atom.
[Embodiment A3] The Compound N of the present invention, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment A4] The compound according to Embodiment A1, wherein R¹², R¹³, R¹⁴, R¹⁵, and R¹⁶ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment A5] The Compound N of the present invention, wherein R¹² and R¹⁶ are a hydrogen atom, R¹³, R¹⁴ and R¹⁵ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment A6] The compound according to Embodiment A1, wherein R¹² and R¹⁶ are a hydrogen atom, R¹³, R¹⁴, and R¹⁵ are identical to or different from each other, and each represent a C1-C4 chain hydrocarbon group optionally having one or more substituents selected from the group E, a C1-C4 alkoxy group optionally having one or more substituents selected from the group E, a C3-C6 alicyclic hydrocarbon group optionally having one or more substituents selected from the group E, a cyano group, a halogen atom, or a hydrogen atom.
[Embodiment A7] The Compound N of the present invention, wherein R⁹ is a methyl group, a halogen atom, or a hydrogen atom.
[Embodiment A8] The compound according to Embodiment A1, wherein R⁹ is a methyl group, a halogen atom, or a hydrogen atom.
[Embodiment A9] The compound according to Embodiment A2, wherein R⁹ is a methyl group, a halogen atom, or a hydrogen atom.
[Embodiment A10] The compound according to Embodiment A3, wherein R⁹ is a methyl group, a halogen atom, or a hydrogen atom.
[Embodiment A11] The compound according to Embodiment A4, wherein R⁹ is a methyl group, a halogen atom, or a hydrogen atom.
[Embodiment A12] The compound according to Embodiment A5, wherein R⁹ is a methyl group, a halogen atom, or a hydrogen atom.
[Embodiment A13] The compound according to Embodiment A6, wherein R⁹ is a methyl group, a halogen atom, or a hydrogen atom.
[Embodiment A14] The compound according to the Compound N of the present invention, wherein R⁹ is a hydrogen atom.
[Embodiment A15] The compound according to Embodiment A1, wherein R⁹ is a hydrogen atom,
[Embodiment A16] The compound according to Embodiment A2, wherein R⁹ is a hydrogen atom.
[Embodiment A17] The compound according to Embodiment A3, wherein R⁹ is a hydrogen atom.
[Embodiment A18] The compound according to Embodiment A4, wherein R⁹ is a hydrogen atom.
[Embodiment A19] The compound according to Embodiment A5, wherein R⁹ is a hydrogen atom,
[Embodiment A20] The compound according to Embodiment A6, wherein R⁹ is a hydrogen atom.
[Embodiment A21] The compound according to any one of Embodiment A1 to Embodiment A20 or the Compound N of the present invention, wherein X is an oxygen atom or a sulfur atom.
[Embodiment A22] The compound according to any one of Embodiment A1 to Embodiment A20 or the Compound N of the present invention, wherein X is an oxygen atom.
[Embodiment A23] The compound according to any one of Embodiment A1 to Embodiment A20 or the Compound N of the present invention, wherein X is a sulfur atom.

Then, a method for producing the present invention compound W will be described.

### Production Method 1

A compound represented by formula (I) (that is, the Compound Z of the present invention) can be produced by reacting a compound represented by formula (M-1) (hereinafter, referred to as a compound (M-1)) with a compound represented by formula (M-2) (hereinafter, referred to as a compound (M-2)), in the presence of a condensing agent.

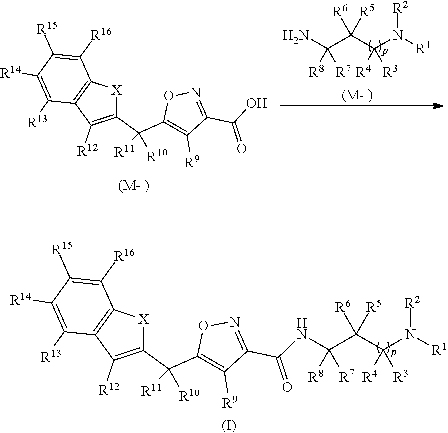

[In the formula, symbols represent the same meaning as described above.]

The reaction is typically performed in a solvent. Examples of the solvent used in the reaction include aromatic hydrocarbons such as benzene and toluene (hereinafter, referred to as aromatic hydrocarbons); aliphatic hydrocarbons such as hexane (hereinafter, referred to as aliphatic hydrocarbons); ethers such as diethyl ether and tetrahydrofuran (hereinafter, referred to as THE) (hereinafter, referred to as ethers); halogenated hydrocarbons such as dichloromethane, chloroform, 1,2-dichloroethane, and chlorobenzene (hereinafter, referred to as halogenated hydrocarbons); acid amides such as dimethylformamide (hereinafter, referred to as DMF) (hereinafter, referred to as acid amides); esters such as ethyl acetate and butyl acetate (hereinafter, referred to as esters); and mixtures thereof.

Examples of the condensing agent used in the reaction include 1-ethyl-3-(3-dimethylaminopropyl) carbodiimide hydrochloride, dicyclohexylcarbodiimide, benzotriazole-1-yloxytris (dimethylamino)phosphonium hexafluorophosphate, and (benzotriazole-1-yloxy)tripyrrolidinophosphonium hexafluorophosphate. The amount of the condensing agent used in the reaction can be typically used in an arbitrary ratio of 1 mol to an excess amount, and is preferably 1 mol to 3 mol, with respect to 1 mol of the compound (M-1).

The reaction may be performed in the presence of a base as necessary. Examples of the base used in the reaction include:
carbonates such as sodium carbonate and potassium carbonate (hereinafter, referred to as a carbonate); tertiary amines such as triethylamine, diisopropylethylamine, 1,8-diazabicyclo[5.4.0]undec-7-ene and 1,5-diazabicyclo[4.3. 0]non-5-ene (hereinafter, referred to as tertiary amines); and nitrogen-containing aromatic compounds such as pyridine and 4-dimethylaminopyridine (hereinafter, referred to as a nitrogen-containing aromatic compound). The amount of the base used in the reaction can be typically used in an arbitrary ratio of 1 mol to an excess amount, and is preferably 1 mol to 3 mol, with respect to 1 mol of the compound (M-1).

The reaction may be performed in the presence of 1-hydroxybenzotriazole, 1-hydroxy-7-azabenzotriazole, N-hydroxysuccinimide, or the like in place of or in addition to the base, as necessary. These amounts are typically used in an arbitrary ratio of 0.01 mol to 1 mol, and preferably in a ratio of 0.05 mol to 0.2 mol, with respect to 1 mol of the compound (M-1).

The reaction time of the reaction is typically in the range of 5 minutes to 72 hours. The reaction temperature of the reaction is typically in the range of −20° C. to 100° C. (however, when the boiling point of the solvent to be used is lower than 100° C., the temperature is −20° C. to the boiling point of the solvent).

In the reaction, the molar ratio of the compound (M-1) to the compound (M-2) used can be arbitrarily set, but is preferably equimolar or a ratio close thereto, for example, a ratio of the compound (M-2) to 1 mol of the compound (M-1) is from 1 mol to 3 mol.

After completion of the reaction, water is added to the reaction mixture, the mixture is extracted with an organic solvent, and the organic layer is subjected to post-treatment operations such as drying and concentration, thereby allowing to provide the Compound Z of the present invention.

The compound (M-2) is a commercially available compound or can be produced according to a known method.

### Production Method 2

The Compound Z of the present invention can also be produced by reacting a compound represented by formula (M-3) (hereinafter, referred to as a compound (M-3)) with the compound (M-2), in the presence of a base.

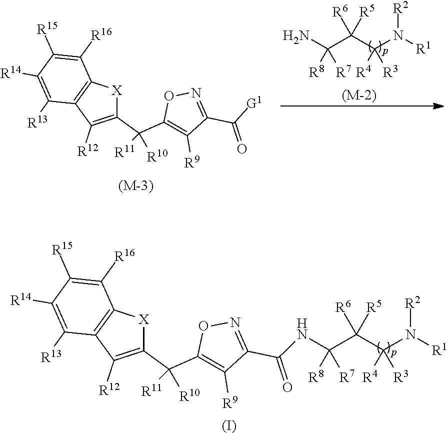

[In the formula, G1 represents a chlorine atom, a bromine atom, or an iodine atom, and other symbols represent the same meaning as described above.]

The reaction is typically performed in a solvent. Examples of the solvent used in the reaction include: ethers; aliphatic hydrocarbons; aromatic hydrocarbons; halogenated hydrocarbons; esters; nitriles such as acetonitrile and butyronitrile (hereinafter, referred to as nitriles); acid amide; sulfoxides such as dimethyl sulfoxide (hereinafter, referred to as DMSO) (hereinafter, referred to as sulfoxides); and mixtures thereof.

Examples of the base used in the reaction include carbonates, tertiary amines, and nitrogen-containing aromatic compounds. The amount of the base used in the reaction can be typically used in an arbitrary ratio of 1 mol to an excess amount, and is preferably 1 mol to 3 mol, with respect to 1 mol of the compound (M-3).

The reaction time of the reaction is typically in the range of 5 minutes to 72 hours. The reaction temperature of the reaction is typically in the range of −20° C. to 100° C.

In the reaction, the molar ratio of the compound (M-2) to the compound (M-3) used can be arbitrarily set, but is preferably equimolar or a ratio close thereto, specifically, from 0.5 mol to 3 mol of the compound (M-2) with respect to 1 mol of the compound (M-3).

After completion of the reaction, water is added to the reaction mixture, the mixture is extracted with an organic solvent, and the organic layer is subjected to post-treatment operations such as drying and concentration, thereby allowing to provide the Compound Z of the present invention.

### Production Method 3

The Compound Z of the present invention can also be produced by reacting a compound represented by formula (M-4) (hereinafter, referred to as a compound (M-4)) with the compound (M-2).

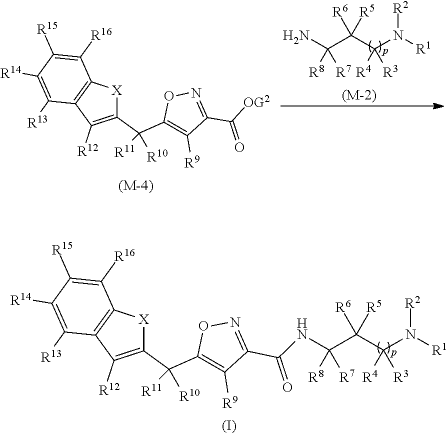

[In the formula, G² represents a methyl group or an ethyl group, and other symbols represent the same meaning as described above.]

The reaction is performed in a solvent or in the absence of a solvent. Examples of the solvent used in the reaction include ethers, aliphatic hydrocarbons, aromatic hydrocarbons, halogenated hydrocarbons, esters, nitriles, acid amides, sulfoxides, and mixtures thereof.

The reaction time of the reaction is typically in the range of 5 minutes to 72 hours. The reaction temperature of the reaction is typically in the range of 50° C. to 200° C.

In the reaction, the molar ratio of the compound (M-2) to the compound (M-4) used can be arbitrarily set, but is preferably equimolar or a ratio close thereto, specifically, from 0.5 mol to 3 mol of the compound (M-2) with respect to 1 mol of the compound (M-4).

After completion of the reaction, water is added to the reaction mixture, the mixture is extracted with an organic solvent, and the organic layer is subjected to post-treatment operations such as drying and concentration, thereby allowing to provide the Compound Z of the present invention,

### Production Method 4

The Compound Z of the present invention can also be produced by reacting a compound represented by formula (M-5) (hereinafter, referred to as a compound (M-5)) with a compound represented by formula (M-6) (hereinafter, referred to as a compound (M-6)), in the presence of a metal catalyst and a base.

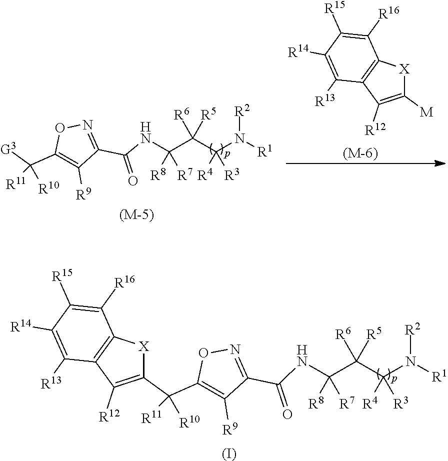

[In the formula, M represents B (OG⁴)₂ or a 4,4,5,5-tetramethyl-1,3,2-dioxaborolane-2-yl group, G⁴ represents a hydrogen atom or a C1 to C6 alkyl group, G³ represents a (dimethoxyphosphinyl)oxy group, a (diethoxyphosphinyl)oxy group, a bromine atom, or an iodine atom, and other symbols represent the same meaning as described above.]

The reaction is typically performed in a solvent. Examples of the solvent used in the reaction include ethers; hydrocarbons; aprotic polar solvents such as DME, N-methylpyrrolidone, and DMSO (hereinafter, referred to as an aprotic polar solvent), water, and mixtures thereof.

Examples of the metal catalyst used in the reaction include palladium catalysts such as tetrakis(triphenylphosphine) palladium (0), 1,1′-bis(diphenylphosphino) ferrocene palladium (II) dichloride, tris(dibenzylideneacetone) dipalladium (0), and palladium (II) acetate; and nickel catalysts such as bis(cyclooctadiene) nickel (0).

Examples of the base used in the reaction include alkali metal carbonates and organic bases.

In the reaction, a ligand may be used as necessary. Examples of the ligand used in the reaction include triphenylphosphine, xantphos, 2,2′-bis(diphenylphosphino)-1,1′-binaphthyl, 1,1′-bis(diphenylphosphino) ferrocene, 2-dicyclohexylphosphino-2′, 4′, 6′-triisopropylbiphenyl, 2-dicyclohexylphosphino-2′, 6′-dimethoxybiphenyl and 1,2-bis (diphenylphosphino) ethane.

For the reaction, the compound (M-6) is typically used in a ratio of 0.5 mol to 10 mol, the metal catalyst is typically used in a ratio of 0.001 mol to 0.5 mol, and the base is typically used in a ratio of 0.1 mol to 5 mol, with respect to 1 mol of the compound (M-5).

When a ligand is used, the ligand is typically used in a ratio of 0.01 mol to 1 mol with respect to 1 mol of the compound (M-5).

The reaction time is typically in the range of 5 minutes to 48 hours. The reaction temperature is typically in the range of −20° C. to 200° C.

After completion of the reaction, water is added to the reaction mixture, the mixture is extracted with an organic solvent, and the organic layer is subjected to post-treatment operations such as drying and concentration, thereby allowing to provide the Compound Z of the present invention.

The compound (M-6) is a commercially available compound or can be produced according to a known method.

Hereinafter, a method for producing a production intermediate will be described.

### Reference Production Method 1

The compound (M-1) can be produced by subjecting the compound (M-4) to a hydrolysis reaction in the presence of a base.

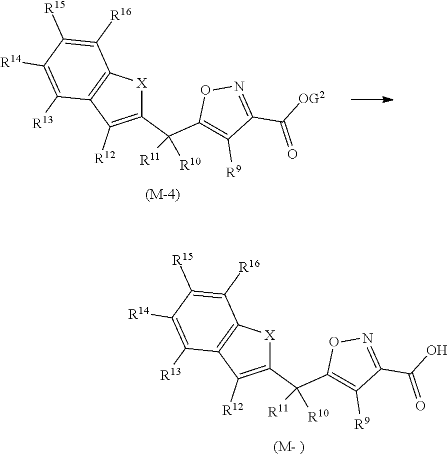

[In the formula, symbols represent the same meaning as described above.]

The reaction is performed in the presence of a base and in the presence of water and an organic solvent. Examples of the solvent used in the reaction include ethers; nitriles; alcohols such as methanol, ethanol, and propanol; and mixtures thereof.

Examples of the base used in the reaction include alkali metal hydroxides such as lithium hydroxide, sodium hydroxide, and potassium hydroxide.

The reaction time of the reaction is typically in the range of 5 minutes to 72 hours. The reaction temperature of the reaction is typically in the range of 0° C. to 100° C.

The amount of the base used in the reaction can be typically used in an arbitrary ratio of 1 mol to an excess amount, and is preferably 1 mol to 5 mol, with respect to 1 mol of the compound (M-1).

After completion of the reaction, water is added to the reaction mixture, the mixture is extracted with an organic solvent, and the organic layer is subjected to post-treatment operations such as drying and concentration, thereby allowing to provide the compound (M-1).

### Reference Production Method 2

The compound (M-3) can be produced by reacting the compound (M-1) with a halogenating agent.

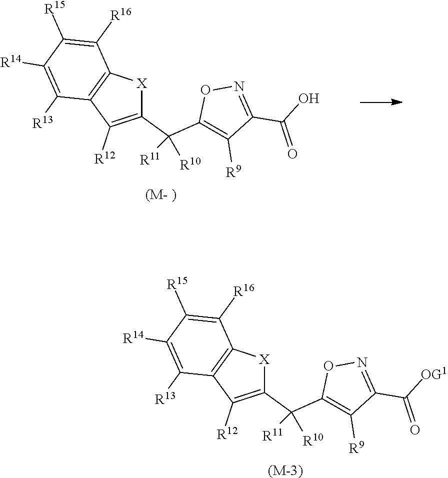

[In the formula, symbols represent the same meaning as described above.]

The reaction is typically performed in a solvent. Examples of the solvent used in the reaction include ethers, aliphatic hydrocarbons, aromatic hydrocarbons, halogenated hydrocarbons, and mixtures thereof.

Examples of the halogenating agent used in the reaction include thionyl chloride, oxalyl chloride, phosphorus oxychloride, phosphorus oxybromide, and phosphorus tribromide.

The reaction time of the reaction is typically in the range of 5 minutes to 24 hours. The reaction temperature is typically in the range of 0 to 100° C.

The amount of the halogenating agent used in the reaction can be typically used in an arbitrary ratio of 1 mol to an excess amount, and is preferably 1 mol to 5 mol, with respect to 1 mol of the compound (M-1).

After completion of the reaction, the reaction mixture is subjected to post-treatment operations such as concentration as it is, whereby the compound (M-3) can be obtained.

### Reference Production Method 3

The compound (M-4) can be produced by reacting the compound (M-7) with the compound (M-6), in the presence of a metal catalyst and a base,

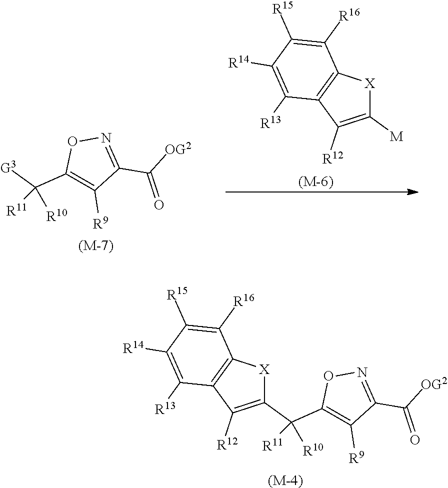

[In the formula, symbols represent the same meaning as described above.]

The reaction can be performed in accordance wit.

Production Method 4, using the compound (M-7) in place of the compound (M-5).

The compound (M-7) is known, or can be produced in accordance with the methods described in WO 2014/119696 A, WO 2010/142801 A, WO 2005/026133 A, and Bioorganic & Medicinal Chemistry Letters, 2010, 20, 1263.

### Reference Production Method 4

The compound (M-5) can be produced by reacting a compound represented by formula (M-8) (hereinafter, referred to as a compound (M-8)) with a phosphorylating agent or a halogenating agent.

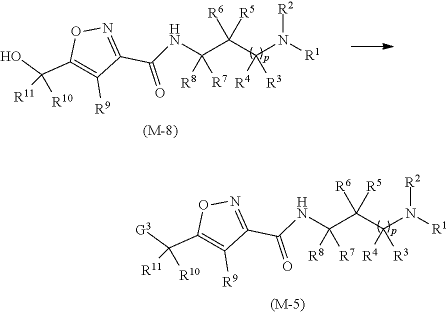

[In the formula, symbols represent the same meaning as described above.]

The reaction is typically performed in a solvent. Examples of the solvent used in the reaction include ethers, aliphatic hydrocarbons, aromatic hydrocarbons, halogenated hydrocarbons, and mixtures thereof.

Examples of the phosphorylating agent used in the reaction include dimethyl chlorophosphate and diethyl chlorophosphate.

Examples of the halogenating agent used in the reaction include phosphorus tribromide, phosphorus oxybromide, a mixture of carbon tetrabromide and triphenylphosphine, and a mixture of iodine and triphenylphosphine.

The reaction time of the reaction is typically in the range of 5 minutes to 24 hours. The reaction temperature is typically in the range of −20 to 100° C.

The amount of the phosphorylating agent or the halogenating agent used in the reaction can be typically used in an arbitrary ratio of 1 mol to an excess amount, and is preferably 1 mol to 5 mol, with respect to 1 mol of the compound (M-8).

After completion of the reaction, water is added to the reaction mixture, the mixture is extracted with an organic solvent, and the organic layer is subjected to post-treatment operations such as drying and concentration, thereby allowing to provide the compound (M-5).

### Reference Production Method 5

The compound (M-8) can be produced by reacting a compound represented by formula (M-9) (hereinafter, referred to as a compound (M-9)) with the compound (M-8).

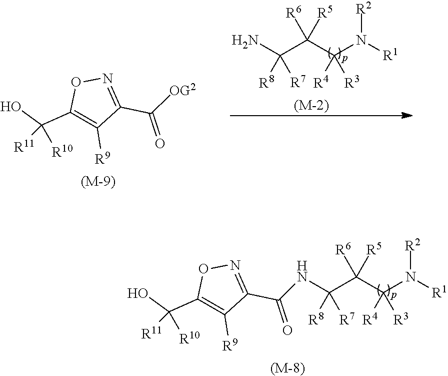

[In the formula, symbols represent the same meaning as described above.]

The reaction can be performed in accordance with Production method 3, using the compound (M-9) in place of the compound (M-4).

The compound (M-9) is a commercially available compound, or can be produced according to a known method.

The Compound of the present invention or the Compound W of the present invention may be mixed with or used in combination with one or more ingredient(s) selected from the group consisting of the following Group (a), Group (b), Group (c), and Group (d) (hereinafter referred to as “Present ingredient”).

When the Compound of the present invention or the Compound W of the present invention is mixed with or used in combination with the Present ingredient, they are used simultaneously, separately, or at time intervals with each other.

When the Compound of the present invention or the Compound W of the present invention is used simultaneously with the Present ingredient, the Compound of the present invention or the Compound W of the present invention and the Present ingredient may be contained in separate formulations or contained in one formulation.

One aspect of the present invention provides a composition comprising one or more ingredient(s) selected from the group consisting of Group (a), Group (b), Group (c), and Group (d), and the Compound of the present invention.

One aspect of the present invention provides a composition comprising one or more ingredient(s) selected from the group consisting of Group (a), Group (b), Group (c), and Group (d), and the Compound W of the present invention (hereinafter referred to as “Composition A”).
Group (a) is a group consisting of acetylcholinesterase inhibitors (for example, carbamate insecticides and organophosphate insecticides), GABA-gated chloride channel blockers (for example, phenylpyrazole insecticides), sodium channel modulators (for example, pyrethroid insecticides), nicotinic acetylcholine receptor competitive modulators (for example, neonicotinoid insecticides), nicotinic acetylcholine receptor allosteric modulators, glutamate-gated chloride channel allosteric modulators (for example, macrolide insecticides), juvenile hormone mimics, multisite inhibitors, chordotonal organ TRPV channel modulators, mite growth inhibitors, microbial disruptors of insect midgut membranes, inhibitors of mitochondrial ATP synthase, uncouplers of oxidative phosphorylation, nicotinic acetylcholine receptor channel blockers (for example, nereistoxin insecticides), inhibitors of chitin biosynthesis, moulting disruptors, ecdysone receptor agonists, octopamine receptor agonists, mitochondrial complexes I, II, III, and IV electron transport inhibitors, voltage-dependent sodium channel blockers, inhibitors of acetyl CoA carboxylase, ryanodine receptor modulators (for example, diamide insecticides), chordotonal organ modulators, and microbial insecticides, and other insecticidal active ingredients, miticidal active ingredients, and nematicidal active ingredients. These ingredients are described in the classification on the basis of action mechanism by IRAC.
Group (b) is a group consisting of nucleic acids synthesis inhibitors (for example, phenylamide fungicides and acylamino acid fungicides), cell division and cytoskeleton inhibitors (for example, MBC fungicides), respiration inhibitors (for example, QoI fungicides, and QiI fungicides), amino acids synthesis and protein synthesis inhibitors (for example, anilino-pyrimidine fungicides), signal transduction inhibitors, lipid synthesis and membrane synthesis inhibitors, sterol biosynthesis inhibitors (for example, DMI fungicides such as triazole fungicides), cell wall biosynthesis inhibitors, melanin synthesis inhibitors, plant defense inducers, fungicides with multi-site contact activity, microbial fungicides, and other fungicidal active ingredients. These ingredients are described in the classification on the basis of action mechanism by FRAC.
Group (c) is a group of plant growth regulatory ingredients (including mycorrhizal fungi and root nodule bacteria).
Group (d) is a group of repellent ingredients.

Hereinafter, examples of the combination of the Present ingredient and the Compound W of the present invention (including the Compound of the present invention) are described. For example, “alanycarb+SX” indicates a combination of alanycarb and SX.

The abbreviation of “SX” indicates any one of the Compound W of the present invention selected from the Compound groups SX1 to SX1844 described in Examples. Also, all of the following Present ingredient are known ingredients, and may be obtained from commercially available formulations, or may be prepared by known methods. When the Present ingredient is a microorganism, it may also be available from a bacterial authority depository. Further, the number in parentheses represents the CAS RN (registered trademark).

Combinations of the Present ingredient in the above Group (a) and the Compound W of the present invention:
abamectin SX, acephate+SX, acequinocyl+SX, acetamiprid+SX, acetoprole+SX, acrinathrin+SX, acynonapyr+SX, afidopyropen+SX, afoxolaner+SX, alanycarb+SX, aldicarb+SX, allethrin+SX, alpha-cypermethrin+SX, alpha-endosulfan+-SX, aluminium phosphide+SX, amitraz+SX, azadirachtin+SX, azamethiphos+SX, azinphos-ethyl+SX, azinphos-methyl+SX, azocyclotin+SX, bark of Celastrus angulatus+SX, bendiocarb+SX, benfluthrin+SX, benfuracarb+SX, bensultap+SX, benzoximate+SX, benzpyrimoxan+SX, beta-cyfluthrin+SX, beta-cypermethrin+SX, bifenazate+SX, bifenthrin+SX, bioallethrin+SX, bioresmethrin+SX, bistrifluron+SX, borax+SX, boric acid+SX, broflanilide+SX, bromopropylate+SX, buprofezin+SX, butocarboxim+SX, butoxycarboxim+SX, cadusafos+SX, calcium phosphide+SX, carbaryl+SX, carbofuran+SX, carbosulfan+SX, cartap hydrochloride+SX, cartap+SX, chinomethionat+SX, chlorantraniliprole+SX, chlordane+SX, chlorethoxyfos+SX, chlorfenapyr+SX, chlorfenvinphos+SX, chlorfluazuron+SX, chlormephos+SX, chloropicrin+SX, chlorpyrifos+SX, chlorpyrifos-methyl+SX, chromafenozide+SX, clofentezine+SX, clothianidin+SX, concanamycin A+SX, coumaphos+SX, cryolite+SX, cyanophos+SX, cyantraniliprole+SX, cyclaniliprole+SX, cyclobutrifluram+SX, cycloprothrin+SX, cycloxaprid+SX, cyenopyrafen+SX, cyetpyrafen+SX, cyflumetofen+SX, cyfluthrin+SX, cyhalodiamide+SX, cyhalothrin+SX, cyhexatin+SX, cypermethrin+SX, cyphenothrin+SX, cyproflanilide+SX, cyromazine+SX, dazomet+SX, deltamethrin+SX, demeton-S-methyl+SX, diafenthiuron+SX, diazinon+SX, dichlorvos+SX, dicloromezotiaz+SX, dicofol+SX, dicrotophos+SX, diflovidazin+SX, diflubenzuron+SX, dimefluthrin+SX, dimethoate+SX, dimethylvinphos+SX, dimpropyridaz+SX, dinotefuran+SX, disodium octaborate+SX, disulfoton+SX, DNOC(2-methyl-4,6-dinitrophenol)+SX, doramectin+SX, dried leaves of Dryopteris filix-mas+SX, emamectin-benzoate+SX, empenthrin+SX, endosulfan+SX, EPN(O-ethyl O-(4-nitrophenyl)phenylphosphonothioate)+SX, epsilon-metofluthrin+SX, epsilon-momfluorothrin+SX, esfenvalerate+SX, ethiofencarb+SX, ethion+SX, ethiprole+SX, ethoprophos+SX, etofenprox+SX, etoxazole+SX, extract of Artemisia absinthium+SX, extract of Azadirachta indica+SX, extract of Cassia nigricans+SX, extract of clitoria ternatea+SX, extract of Symphytum officinale+SX, extract of Chenopodium ambrosioides+SX, extract of Tanacetum vulgare+SX, extract of Urtica dioica+SX, extract of Viscum album+SX, famphur+SX, fenamiphos+SX, fenazaquin+SX, fenbutatin oxide+SX, fenitrothion+SX, fenmezoditiaz+SX, fenobucarb+SX, fenoxycarb+SX, fenpropathrin+SX, fenpyroximate+SX, fenthion+SX, fenvalerate+SX, fipronil+SX, flometoquin+SX, flonicamid+SX, fluacrypyrim+SX, fluazaindolizine+SX, fluazuron+SX, flubendiamide+SX, fluchlordiniliprole+SX, flucycloxuron+SX, flucythrinate+SX, fluensulfone+SX, flufenoprox+SX, flufenoxuron+SX, flufiprole+SX, flumethrin+SX, flupentiofenox+SX, flupyradifurone+SX, flupyrimin+SX, flupyroxystrobin+SX, fluralaner+SX, fluvalinate+SX, fluxametamide+SX, formetanate+SX, fosthiazate+SX, furamethrin+SX, furathiocarb+SX, gamma-cyhalothrin+SX, GS-omega/kappa HXTX-Hvla peptide+SX, halfenprox+SX, halofenozide+SX, heptafluthrin+SX, heptenophos+SX, hexaflumuron+SX, hexythiazox+SX, potassium salt of hop beta acid+SX, hydramethylnon+SX, hydroprene+SX, imicyafos+SX, imidacloprid+SX, imidaclothiz+SX, imiprothrin+SX, indazapyroxamet+SX, indoxacarb+SX, isocycloseram+SX, isofenphos+SX, isoprocarb+SX, isopropyl-O-(methoxyaminothiophosphoryl) salicylate+SX, isoxathion+SX, ivermectin+SX, kadethrin+SX, kappa-tefluthrin+SX, kappa-bifenthrin+SX, kinoprene+SX, lambda-cyhalothrin+SX, ledprona+SX, lenoremycin+SX, lepimectin+SX, lime sulfur+SX, lotilaner+SX, lufenuron+SX, machine oil+SX, malathion+SX, mecarbam+SX, meperfluthrin+SX, metaflumizone+SX, metam+SX, methamidophos+SX, methidathion+SX, methiocarb+SX, methomyl+SX, methoprene+SX, methoxychlor+SX, methoxyfenozide+SX, methyl bromide+SX, metofluthrin+SX, metolcarb+SX, metoxadiazone+SX, mevinphos+SX, milbemectin+SX, milbemycin oxime+SX, mivorilaner+SX, modoflaner+SX, momfluorothrin+SX, monocrotophos+SX, moxidectin+SX, naled+SX, nicofluprole+SX, nicotine+SX, nicotine-sulfate+SX, nitenpyram+SX, novaluron+SX, noviflumuron+SX, oil of the seeds of Chenopodium anthelminticum+SX, omethoate+SX, oxamyl+SX, oxazosulfyl+SX, oxydemeton-methyl+SX, parathion+SX, parathion-methyl+SX, permethrin+SX, phenothrin+SX, phenthoate+SX, phorate+SX, phosalone+SX, phosmet+SX, phosphamidon+SX, phosphine+SX, phoxim+SX, pirimicarb+SX, pirimiphos-methyl+SX, prallethrin+SX, profenofos+SX, profluthrin+SX, propargite+SX, propetamphos+SX, propoxur+SX, propylene glycol alginate+SX, prothiofos+SX, pyflubumide+SX, pymetrozine+SX, pyraclofos+SX, pyrethrins+SX, pyridaben+SX, pyridalyl+SX, pyridaphenthion+SX, pyrifluquinazone+SX, pyrimidifen+SX, pyriminostrobin+SX, pyriprole+SX, pyriproxyfen+SX, quinalphos+SX, resmethrin+SX, rotenone+SX, ryanodine+SX, sarolaner+SX, selamectin+SX, sigma-cypermethrin+SX, silafluofen+SX, sodium borate+SX, sodium metaborate+SX, spidoxamat+SX, spinetoram+SX, spinosad+SX, spirobudifen+SX, spirodiclofen+SX, spiromesifen+SX, spiropidion+SX, spirotetramat+SX, sulfiflumin+SX, sulfluramid+SX, sulfotep+SX, sulfoxaflor+SX, sulfur+SX, sulfuryl fluoride+SX, tartar emetic+SX, tau-fluvalinate+SX, tebufenozide+SX, tebufenpyrad+SX, tebupirimfos+SX, teflubenzuron+SX, tefluthrin+SX, temephos+SX, terbufos+SX, terpene constituents of the extract of chenopodium ambrosioides near ambrosioides+SX, tetrachlorantraniliprole+SX, tetrachlorvinphos+SX, tetradifon+SX, tetramethrin+SX, tetramethylfluthrin+SX, tetraniliprole+SX, theta-cypermethrin+SX, thiacloprid+SX, thiamethoxam+SX, thiocyclam+SX, thiodicarb+SX, thiofanox+SX, thiometon+SX, thiosultap-disodium+SX, thiosultap-monosodium+SX, tigolaner+SX, tiorantraniliprole+SX, tioxazafen+SX, tolfenpyrad+SX, tralomethrin+SX, transfluthrin+SX, triazamate+SX, triazophos+SX, trichlorfon+SX, trifluenfuronate+SX, triflumezopyrim+SX, triflumuron+SX, trimethacarb+SX, tyclopyrazoflor+SX, umifoxolaner+SX, vamidothion+SX, wood extract of Quassia amara+SX, XMC (3,5-dimethylphenyl N-methylcarbamate)+SX, xylylcarb+SX, zeta-cypermethrin+SX, zinc phosphide+SX, 4-[5-(3,5-dichlorophenyl)-5-(trifluoromethyl)-4,5-dihydro-1,2-oxazol-3-yl]-2-methyl-N-(1-oxothietan-3-yl)benzamide (1241050-20-3)+SX, 3-methoxy-N-(5-{5-(trifluoromethyl)-5-[3-(trifluoromethyl)phenyl]-4,5-dihydro-1,2-oxazol-3-yl}indan-1-yl)propanamide (1118626-57-5)+SX, N-{4-chloro-3-[(1-cyanocyclopropyl)(carbamoyl]phenyl}-1-methyl-4-(methanesulfonyl)-3-(1,1,2,2,2-pentafluoroethyl)-1H-pyrazole-3-carboxamide (1400768-21-9)+SX, N-[3-chloro-1-(pyridin-3-yl)-1H-pyrazol-4-yl]-2-(methanesulfonyl)propanamide (2396747-83-2)+SX, N-[4-chloro-2-(pyridin-3-yl)-1,3-thiazol-5-yl]-N-ethyl-3-(methanesulfonyl)propanamide+SX, 1,4-dimethyl-2-[2-(pyridin-3-yl)-2H-indazol-5-yl]-1,2,4-triazolidine-3,5-dione (2171099-09-3)+SX, 2-isopropyl-5-[(3,4,4-trifluoro-3-buten-1-yl)sulfonyl]-1,3,4-thiadiazole (2058052-95-0)+SX, N-({2-fluoro-4-[(2S,3S)-2-hydroxy-3-(3,4,5-trichlorophenyl)-3-(trifluoromethyl)pyrrolidin-1-yl]phenyl}methyl)cyclopropanecarboxamide+SX, 7-fluoro-N-[1-(methylsulfanyl)-2-methylpropan-2-yl]-2-(pyridin-3-yl)-2H-indazole-4-carboxamide+SX, 7-fluoro-N-[1-(methanesulfinyl)-2-methylpropan-2-yl]-2-(pyridin-3-yl)-2H-indazole-4-carboxamide+SX, 7-fluoro-N-[1-(methanesulfonyl)-2-methylpropan-2-yl]-2-(pyridin-3-yl)-2H-indazole-4-carboxamide+SX, N-[1-(difluoromethyl)cyclopropyl]-2-(pyridin-3-yl)-2H-indazole-4-carboxamide+SX, 2,9-dihydro-9-(methoxymethyl)-2-(pyridin-3-yl)-10H-pyrazolo[3,4-f]pyrido[2,3-b][1,4]oxazepin-10-one (2607927-97-7)+SX, BT crop protein CrylAb+SX, BT crop protein CrylAc+SX, BT crop protein Cry1Fa+SX, BT crop protein Cry1A.105+SX, BT crop protein Cry2Ab+SX, BT crop protein Vip3A+SX, BT crop protein mCry3A+SX, BT crop protein Cry3Ab+SX, BT crop protein Cry3Bb+SX, BT crop protein Cry34Abl/Cry35Abl+SX, Adoxophyes orana granulosis virus strain BV-0001+SX, Anticarsia gemmatalis MNPV(multiple nucleocapsid nucleopolyhedrovirus)+SX, Autographa californica MNPV+SX, Cydia pomonella GV (granulovirus) strain V15+SX, Cydia pomonella GV strain V22+SX, Cryptophlebia leucotreta GV+SX, Dendrolimus punctatus cypovirus+SX, Helicoverpa armigera NPV (nucleopolyhedrovirus) strain BV-0003+SX, Helicoverpa zea NPV+SX, Lymantria dispar NPV+SX, Mamestra brassicae NPV+SX, Mamestra configurata NPV+SX, Neodiprion abietis NPV+SX, Neodiprion lecontei NPV+SX, Neodiprion sertifer NPV+SX, Nosema locustae+SX, Orgyia pseudotsugata NPV+SX, Pieris rapae GV+SX, Plodia interpunctella GV+SX, Spodoptera exigua MNPV+SX, Spodoptera littoralis MNPV+SX, Spodoptera litura NPV+SX, Arthrobotrys dactyloides+SX, Bacillus firmus strain GB-126+SX, Bacillus firmus strain I-1582+SX, Bacillus firmus strain NCIM2637+SX, Bacillus megaterium+SX, Bacillus sp. strain AQ175+SX, Bacillus sp. strain AQ177+SX, Bacillus sp. strain AQ178+SX, Bacillus sphaericus strain 2362 serotype H5a5b+SX, Bacillus sphaericus strain ABTS1743+SX, Bacillus thuringiensis strain AQ52+SX, Bacillus thuringiensis strain BD #32+SX, Bacillus thuringiensis strain CR-371+SX, Bacillus thuringiensis subsp. Aizawai strain ABTS-1857+SX, Bacillus thuringiensis subsp. Aizawai strain AM65-52+SX, Bacillus thuringiensis subsp. Aizawai strain GC-91+SX, Bacillus thuringiensis subsp. Aizawai strain NB200+SX, Bacillus thuringiensis subsp. Aizawai Serotype strain H-7+SX, Bacillus thuringiensis subsp. Kurstaki strain ABTS351+SX, Bacillus thuringiensis subsp. Kurstaki strain BMP123+SX, Bacillus thuringiensis subsp. Kurstaki strain CCT1306)+SX, Bacillus thuringiensis subsp. Kurstaki strain EG2348+SX, Bacillus thuringiensis subsp. Kurstaki strain EG7841+SX, Bacillus thuringiensis subsp. Kurstaki strain EVB113-19+SX, Bacillus thuringiensis subsp. Kurstaki strain F810+SX, Bacillus thuringiensis subsp. Kurstaki strain HD-1+SX, Bacillus thuringiensis subsp. Kurstaki strain PB54+SX, Bacillus thuringiensis subsp. Kurstaki strain SA-11+SX, Bacillus thuringiensis subsp. Kurstaki strain SA-12+SX, Bacillus thuringiensis subsp. Tenebriosis strain NB176+SX, Bacillus thuringiensis subsp. Thuringiensis strain MPPL002+SX, Bacillus thuringiensis subsp. morrisoni+SX, Bacillus thuringiensis var. colmeri+SX, Bacillus thuringiensis var. darmstadiensis strain 24-91+SX, Bacillus thuringiensis var. dendrolimus+SX, Bacillus thuringiensis var. galleriae+SX, Bacillus thuringiensis var. israelensis strain BMP144+SX, Bacillus thuringiensis var. israelensis serotype strain H-14+SX, Bacillus thuringiensis var. japonensis strain buibui+SX, Bacillus thuringiensis var. san diego strain M-7+SX, Bacillus thuringiensis var. 7216+SX, Bacillus thuringiensis var. aegypti+SX, Bacillus thuringiensis var. T36+SX, Beauveria bassiana strain ANT-03+SX, Beauveria bassiana strain ATCC74040+SX, Beauveria bassiana strain GHA+SX, Beauveria brongniartii+SX, Burkholderia rinojensis strain A396+SX, Chromobacterium subtsugae strain PRAA4-1T+SX, Dactyllela ellipsospora+SX, Dectylaria thaumasia+SX, Hirsutella minnesotensis+SX, Hirsutella rhossiliensis+SX, Hirsutella thompsonii+SX, Lagenidium giganteum+SX, Lecanicillium lecanii strain KV01+SX, Lecanicillium lecanii conidia of strain DAOM198499+SX, Lecanicillium lecanii conidia of strain DAOM216596+SX, Lecanicillium muscarium strain Ve6+SX, Metarhizium anisopliae strain F52+SX, Metarhizium anisopliae var. acridum+SX, Metarhizium anisopliae var. anisopliae BIPESCO 5/F52+SX, Metarhizium flavoviride+SX, Monacrosporium phymatopagum+SX, Paecilomyces fumosoroseus Apopka strain 97+SX, Paecilomyces lilacinus strain 251+SX, Paecilomyces tenuipes strain T1+SX, Paenibacillus popilliae+SX, Pasteuria nishizawae strain Pn1+SX, Pasteuria penetrans+SX, Pasteuria usgae+SX, Pasteuria thornei+SX, Serratia entomophila+SX, Verticillium chlamydosporium+SX, Verticillium lecani strain NCIM1312+SX, Wolbachia pipientis+SX.

Combination of the Present ingredient in the above Group (b) and the Compound W of the present invention: acibenzolar-S-methyl+SX, aldimorph+SX, ametoctradin+SX, aminopyrifen+SX, amisulbrom+SX, anilazine+SX, azaconazole+SX, azoxystrobin+SX, basic copper sulfate+SX, benalaxyl+SX, benalaxyl-M+SX, benodanil+SX, benomyl+SX, benthiavalicarb+SX, benthiavalicarb-isopropyl+SX, benzovindiflupyr+SX, binapacryl+SX, biphenyl+SX, bitertanol+SX, bixafen+SX, blasticidin-S+SX, Bordeaux mixture+SX, boscalid+SX, bromothalonil+SX, bromuconazole+SX, bupirimate+SX, captafol+SX, captan+SX, carbendazim+SX, carboxin+SX, carpropamid+SX, chinomethionat+SX, chitin+SX, chloroinconazide+SX, chloroneb+SX, chlorothalonil+SX, chlozolinate+SX, colletochlorin B+SX, copper(II) acetate+SX, copper(II) hydroxide+SX, copper oxychloride+SX, copper(II) sulfate+SX, coumoxystrobin+SX, cyazofamid+SX, cyflufenamid+SX, cymoxanil+SX, cyproconazole+SX, cyprodinil+SX, dichlobentiazox+SX, dichlofluanid+SX, diclocymet+SX, diclomezine+SX, dicloran+SX, diethofencarb+SX, difenoconazole+SX, diflumetorim+SX, dimethachlone+SX, dimethirimol+SX, dimethomorph+SX, dimoxystrobin+SX, diniconazole+SX, diniconazole-M+SX, dinocap+SX, dipotassium hydrogenphosphite+SX, dipymetitrone+SX, dithianon+SX, dodecylbenzenesulphonic acid bisethylenediamine copper(II) salt+SX, dodemorph+SX, dodine+SX, edifenphos+SX, enoxastrobin+SX, epoxiconazole+SX, etaconazole+SX, ethaboxam+SX, ethirimol+SX, etridiazole+SX, extract of Allium sativum+SX, extract of the cotyledons of lupine plantlets (“BLAD”)+SX, extract of Equisetum arvense+SX, extract of Melaleuca alternifolia+SX, extract of Reynoutria sachalinensis+SX, extract of Tropaeolum majus+SX, famoxadone+SX, fenamidone+SX, fenaminstrobin+SX, fenarimol+SX, fenbuconazole+SX, fenfuram+SX, fenhexamid+SX, fenoxanil+SX, fenpiclonil+SX, fenpicoxamid+SX, fenpropidin+SX, fenpropimorph+SX, fenpyrazamine+SX, fentin acetate+SX, fentin chloride+SX, fentin hydroxide+SX, ferbam+SX, ferimzone+SX, florylpicoxamid+SX, fluazinam+SX, flubeneteram+SX, fludioxonil+SX, flufenoxadiazam+SX, flufenoxystrobin+SX, fluindapyr+SX, flumetylsulforim+SX, flumorph+SX, fluopicolide+SX, fluopyram+SX, fluopimomide+SX, fluoroimide+SX, fluoxapiprolin+SX, fluoxastrobin+SX, fluoxytioconazole+SX, fluquinconazole+SX, flusilazole+SX, flusulfamide+SX, flutianil+SX, flutolanil+SX, flutriafol+SX, fluxapyroxad+SX, folpet+SX, fosetyl+SX, fosetyl-aluminium+SX, fuberidazole+SX, furalaxyl+SX, furametpyr+SX, guazatine+SX, hexaconazole+SX, hymexazole+SX, imazalil+SX, imibenconazole+SX, iminoctadine+SX, iminoctadine triacetate+SX, inpyrfluxam+SX, iodocarb+SX, ipconazole+SX, ipfentrifluconazole+SX, ipflufenoquin+SX, iprobenfos+SX, iprodione+SX, iprovalicarb+SX, isofetamid+SX, isoflucypram+SX, isoprothiolane+SX, isopyrazam+SX, isotianil+SX, kasugamycin+SX, kresoxim-methyl+SX, laminarin+SX, leaves and bark of Quercus+SX, mancozeb+SX, mandestrobin+SX, mandipropamid+SX, maneb+SX, mefentrifluconazole+SX, mepanipyrim+SX, mepronil+SX, meptyldinocap+SX, metalaxyl+SX, metalaxyl-M+SX, metarylpicoxamid+SX, metconazole+SX, methasulfocarb+SX, metiram+SX, metominostrobin+SX, metrafenone+SX, metyltetraprole+SX, myclobutanil+SX, naftifine+SX, nuarimol+SX, octhilinone+SX, ofurace+SX, orysastrobin+SX, oxadixyl+SX, oxathiapiprolin+SX, oxine-copper+SX, oxolinic acid+SX, oxpoconazole+SX, oxpoconazole fumarate+SX, oxycarboxin+SX, oxytetracycline+SX, pefurazoate+SX, penconazole+SX, pencycuron+SX, penflufen+SX, penthiopyrad+SX, phenamacril+SX, phosphorous acid+SX, phthalide+SX, picarbutrazox+SX, picoxystrobin+SX, piperalin+SX, polyoxins+SX, potassium hydrogencarbonate+SX, potassium dihydrogenphosphite+SX, probenazole+SX, prochloraz+SX, procymidone+SX, propamidine+SX, propamocarb+SX, propiconazole+SX, propineb+SX, proquinazid+SX, prothiocarb+SX, prothioconazole+SX, pydiflumetofen+SX, pyraclostrobin+SX, pyrametostrobin+SX, pyraoxystrobin+SX, pyrapropoyne+SX, pyraziflumid+SX, pyrazophos+SX, pyribencarb+SX, pyributicarb+SX, pyridachlometyl+SX, pyrifenox+SX, pyrimethanil+SX, pyrimorph+SX, pyriofenone+SX, pyrisoxazole+SX, pyroquilon+SX, Quillaja extract+SX, quinconazole+SX, quinofumelin+SX, quinoxyfen+SX, quintozene+SX, Saponins of Chenopodium quinoa+SX, seboctylamine+SX, sedaxane+SX, silthiofam+SX, simeconazole+SX, sodium hydrogencarbonate+SX, spiroxamine+SX, streptomycin+SX, sulfur+SX, tebuconazole+SX, tebufloquin+SX, teclofthalam+SX, tecnazene+SX, terbinafine+SX, tetraconazole+SX, thiabendazole+SX, thifluzamide+SX, thiophanate+SX, thiophanate-methyl+SX, thiram+SX, thymol+SX, tiadinil+SX, tolclofos-methyl+SX, tolfenpyrad+SX, tolprocarb+SX, tolylfluanid+SX, triadimefon+SX, triadimenol+SX, triazoxide+SX, triclopyricarb+SX, tricyclazole+SX, tridemorph+SX, trifloxystrobin+SX, triflumizole+SX, triforine+SX, triticonazole+SX, validamycin+SX, valifenalate+SX, vinclozolin+SX, yellow mustard powder+SX, zinc thiazole+SX, zineb+SX, ziram+SX, zoxamide+SX, N′-[4-({3-[(4-chlorophenyl)methyl]-1,2,4-thiadiazol-5-yl}oxy)-2,5-dimethylphenyl]-N-ethyl-N-methylmethanimidamide (1202781-91-6)+SX, N′-{4-[(4,5-dichlorothiazol-2-yl)oxy]-2,5-dimethylphenyl}-N-ethyl-N-methylmethanimidamide (929908-57-6)+SX, N′-(2,5-dimethyl-4-phenoxyphenyl)-N-ethyl-N-methylmethanimidamide (1052688-31-9)+SX, N′-[5-chloro-4-(2-fluorophenoxy)-2-methylphenyl]-N-ethyl-N-methylmethanimidamide (2055589-28-9)+SX, N′-[2-chloro-4-(2-fluorophenoxy)-5-methylphenyl]-N-ethyl-N-methylmethanimidamide (2055756-21-1)+SX, N′-(2-chloro-4-phenoxy-5-methylphenyl)-N-ethyl-N-methylmethanimidamide (2062599-39-5)+SX, N′-[4-(1-hydroxy-1-phenyl-2,2,2-trifluoroethyl)-2-methyl-5-methoxyphenyl]-N-isopropyl-N-methylmethanimidamide (2101814-55-3)+SX, N′-[5-bromo-6-(1-methyl-2-propoxyethoxy)-2-methylpyridin-3-yl]-N-ethyl-N-methylmethanimidamide (1817828-69-5)+SX, 4-(2-bromo-4-fluorophenyl)-N-(2-chloro-6-fluorophenyl)-1,3-dimethyl-1H-pyrazol-5-amine (1362477-26-6)+SX, 2-[6-(3-fluoro-4-methoxyphenyl)-5-methylpyridin-2-yl]quinazoline (1257056-97-5)+SX, ethyl (2Z)-3-amino-2-cyano-3-phenylacrylate (39491-78-6)+SX, N-[(2-chlorothiazol-5-yl)methyl]-N-ethyl-6-methoxy-3-nitropyridin-2-amine (1446247-98-8)+SX, 5-(4-chlorobenzyl)-2-(chloromethyl)-2-methyl-1-(1H-1,2,4-triazol-1-ylmethyl)cyclopentan-1-ol (1394057-11-4)+SX, (1R, 2S, 5S)-5-(4-chlorobenzyl)-2-(chloromethyl)-2-methyl-1-(1H-1,2,4-triazol-1-ylmethyl)cyclopentan-1-ol (1801930-06-2)+SX, (13,2R, 5R)-5-(4-chlorobenzyl)-2-(chloromethyl)-2-methyl-1-(1H-1,2,4-triazol-1-ylmethyl)cyclopentan-1-ol (1801930-07-3)+SX, 2-(chloromethyl)-5-(4-fluorobenzyl)-2-methyl-1-(1H-1,2,4-triazol-1-ylmethyl)cyclopentan-1-ol (1394057-13-6)+SX, (1R, 23,5S)-2-(chloromethyl)-5-(4-fluorobenzyl)-2-methyl-1-(1H-1,2,4-triazol-l-ylmethyl)cyclopentan-1-ol (1801930-08-4)+SX, (1S, 2R, 5R)-2-(chloromethyl)-5-(4-fluorobenzyl)-2-methyl-1-(1H-1,2,4-triazol-1-ylmethyl)cyclopentan-1-ol (1801930-09-5)+SX, methyl 3-[(4-chlorophenyl)methyl]-2-hydroxy-1-methyl-2-(1H-1,2,4-triazol-1-ylmethyl)cyclopentan-1-carboxylate (1791398-02-1)+SX, 1-(2,4-difluorophenyl)-2-(1H-1,2,4-triazol-1-yl)-1-[1-(4-bromo-2,6-difluorophenoxy)cyclopropyl]ethanol (2019215-86-0)+SX, 1-(2,4-difluorophenyl)-2-(1H-1,2,4-triazol-1-yl)-1-[1-(4-chloro-2,6-difluorophenoxy)cyclopropyl]ethanol (2019215-84-8)+SX, 1-[2-(1-chlorocyclopropyl)-3-(2-fluorophenyl)-2-hydroxypropyl]-1H-imidazole-5-carbonitrile (2018316-13-5)+SX, 1-[2-(1-chlorocyclopropyl)-3-(2,3-difluorophenyl)-2-hydroxypropyl]-1H-imidazole-5-carbonitrile (2018317-25-2)+SX, 2-[6-(4-bromophenoxy)-2-(trifluoromethyl)pyridin-3-yl]-1-(1H-1,2,4-triazol-1-yl)propan-2-ol (2082661-43-4)+SX, 2-[6-(4-chlorophenoxy)-2-(trifluoromethyl)pyridin-3-yl]-1-(1H-1,2,4-triazol-1-yl)propan-2-ol (2082660-27-1)+SX, methyl ({2-methyl-5-[1-(4-methoxy-2-methylphenyl)-1H-pyrazol-3-yl]phenyl}methyl)carbamate (1605879-98-8)+SX, 2-(difluoromethyl)-N-[1,1,3-trimethyl-2,3-dihydro-1H-inden-4-yl]pyridine-3-carboxamide (1616239-21-4)+SX, 2-(difluoromethyl)-N-[3-ethyl-1,1-dimethyl-2,3-dihydro-1H-inden-4-yl]pyridine-3-carboxamide (1847460-02-9)+SX, 2-(difluoromethyl)-N-[3-propyl-1,1-dimethyl-2,3-dihydro-1H-inden-4-yl]pyridine-3-carboxamide (1847460-05-2)+SX, (2E,3Z)-5-{[1-(4-chlorophenyl)-1H-pyrazol-3-yl]oxy}-2-(methoxyimino)-N,3-dimethylpent-3-enamide (1445331-27-0)+SX, (2E,3Z)-5-{[1-(2,4-dichlorophenyl)-1H-pyrazol-3-yl]oxy}-2-(methoxyimino)-N,3-dimethylpent-3-enamide (1445331-54-3)+SX, 5-chloro-4-({2-[6-(4-chlorophenoxy)pyridin-3-yl]ethyl}amino)-6-methylpyrimidine (1605340-92-8)+SX, N-(1-benzyl-1,3-dimethylbutyl)-8-fluoroquinoline-3-carboxamide (2132414-04-9)+SX, N-(1-benzyl-3,3,3-trifluoro-1-methylpropyl)-8-fluoroquinoline-3-carboxamide (2132414-00-5)+SX, 4,4-dimethyl-2-({4-[5-(trifluoromethyl)-1,2,4-oxadiazol-3-yl]phenyl}methyl)isoxazolidin-3-one (2098918-25-1)+SX, 5,5-dimethyl-2-({4-[5-(trifluoromethyl)-1,2,4-oxadiazol-3-yl]phenyl}methyl)isoxazolidin-3-one (2098918-26-2)+SX, N-ethyl-2-methyl-N-({4-[5-(trifluoromethyl)-1,2,4-oxadiazol-3-yl]phenyl}methyl)propanamide+SX, N,2-dimethoxy-N-({4-[5-(trifluoromethyl)-1,2,4-oxadiazol-3-yl]phenyl}methyl)propanamide+SX, N-methoxy-N-({4-[5-(trifluoromethyl)-1,2,4-oxadiazol-3-yl]phenyl}methyl)(cyclopropanecarboxamide+SX, N-methoxy-N′-methyl-N-({4-[5-(trifluoromethyl)-1,2,4-oxadiazol-3-yl]phenyl}methyl)urea+SX, N′-ethyl-N-methoxy-N-({4-[5-(trifluoromethyl)-1,2,4-oxadiazol-3-yl]phenyl}methyl)urea+SX, N,N′-dimethoxy-N-({4-[5-(trifluoromethyl)-1,2,4-oxadiazol-3-yl]phenyl}methyl)urea+SX, N-acetyl-2-(ethanesulfonyl)-N-[2-(methoxycarbonyl)-4-(trifluoromethoxy)phenyl]-4-(trifluoromethyl)benzamide (2043675-28-9)+SX, 3-(4-bromo-7-fluoroindol-1-yl)butan-2-yl N-[(3-hydroxy-4-methoxypyridin-2-yl)carbonyl]-L-alaninate+SX, 3-(7-bromoindol-1-yl)butan-2-yl N-[(3-hydroxy-4-methoxypyridin-2-yl)carbonyl]-L-alaninate+SX, 3-(7-bromo-4-fluoroindol-1-yl)butan-2-yl N-[(3-hydroxy-4-methoxypyridin-2-yl)carbonyl]-L-alaninate+SX, 3-(3,5-dichloropyridin-2-yl)butan-2-yl N-[(3-hydroxy-4-methoxypyridin-2-yl)carbonyl]-L-alaninate+SX, 3-(3,5-dichloropyridin-2-yl)butan-2-yl N-{[3-(acetoxymethoxy)-4-methoxypyridin-2-yl]carbonyl}-L-alaninate+SX, (1S)-1-[1-(naphthalen-1-yl)cyclopropyl]ethyl N-[(3-hydroxy-4-methoxypyridin-2-yl)carbonyl]-L-alaninate+SX, (1S)-1-[1-(naphthalen-1-yl)cyclopropyl]ethyl N-[(3-acetoxy-4-methoxypyridin-2-yl)carbonyl]-L-alaninate+SX, (1S)-1-[1-(naphthalen-1-yl)cyclopropyl]ethyl N-{[3-(acetoxymethoxy)-4-methoxypyridin-2-yl]carbonyl}-L-alaninate+SX, Agrobacterium radiobactor strain K1026+SX, N-({4-[5-(trifluoromethyl)-1,2,4-oxadiazol-3-yl]phenyl}methyl)cyclopropanecarboxamide+SX, N-allyl-N-({4-[5-(trifluoromethyl)-1,2,4-oxadiazol-3-yl]phenyl}methyl)acetamide+SX, N-allyl-N-({4-[5-(trifluoromethyl)-1,2,4-oxadiazol-3-yl]phenyl}methyl)propanamide+SX, N-({4-[5-(trifluoromethyl)-1,2,4-oxadiazol-3-yl]phenyl}methyl)propanamide+SX, 3,3,3-trifluoro-N-({2-fluoro-4-[5-(trifluoromethyl)-1,2,4-oxadiazol-3-yl]phenyl}methyl)propanamide+SX, 3,3,3-trifluoro-N-({3-fluoro-4-[5-(trifluoromethyl)-1,2,4-oxadiazol-3-yl]phenyl}methyl)propanamide+SX, 3,3,3-trifluoro-N-({2,3-difluoro-4-[5-(trifluoromethyl)-1,2,4-oxadiazol-3-yl]phenyl}methyl)propanamide+SX, N-({2,3-difluoro-4-[5-(trifluoromethyl)-1,2,4-oxadiazol-3-yl]phenyl}methyl)butanamide+SX, N-methoxy-N-methyl-N′—({4-[5-(trifluoromethyl)-1,2,4-oxadiazol-3-yl]phenyl}methyl)urea+SX, N,N-diethyl-N′—({4-[5-(trifluoromethyl)-1,2,4-oxadiazol-3-yl]phenyl}methyl)urea+SX, N-methyl-N′—({4-[5-(trifluoromethyl)-1,2,4-oxadiazol-3-yl]phenyl}methyl)urea+SX, 1-({4-[5-(trifluoromethyl)-1,2,4-oxadiazol-3-yl]phenyl}methyl)pyrrolidin-2-one+SX, 1-({4-[5-(trifluoromethyl)-1,2,4-oxadiazol-3-yl]phenyl}methyl)piperidin-2-one+SX, 4-({4-[5-(trifluoromethyl)-1,2,4-oxadiazol-3-yl]phenyl}methyl)morpholin-3-one+SX, 2-({4-[5-(trifluoromethyl)-1,2,4-oxadiazol-3-yl]phenyl}methyl)isoxazolidin-3-one+SX, 3,3-dimethyl-1-({4-[5-(trifluoromethyl)-1,2,4-oxadiazol-3-yl]phenyl}methyl)piperidin-2-one+SX, 2-({4-[5-(trifluoromethyl)-1,2,4-oxadiazol-3-yl]phenyl}methyl)-1,2-oxazinan-3-one+SX, 1-({3-fluoro-4-[5-(trifluoromethyl)-1,2,4-oxadiazol-3-yl]phenyl}methyl)azepan-2-one+SX, 4,4-dimethyl-1-({4-[5-(trifluoromethyl)-1,2,4-oxadiazol-3-yl]phenyl}methyl)pyrrolidin-2-one+SX, 5-methyl-1-({4-[5-(trifluoromethyl)-1,2,4-oxadiazol-3-yl]phenyl}methyl)pyrrolidin-2-one+SX, ethyl 1-({4-[5-(trifluoromethyl)-1,2,4-oxadiazol-3-yl]phenyl}methyl)-1H-pyrazole-4-carboxylate+SX, N-methyl-1-({4-[5-(trifluoromethyl)-1,2,4-oxadiazol-3-yl]phenyl}methyl)-1H-pyrazole-4-carboxamide+SX, N-propyl-1-({4-[5-(trifluoromethyl)-1,2,4-oxadiazol-3-yl]phenyl}methyl)-1H-pyrazole-4-carboxamide+SX, N-methoxy-1-({4-[5-(trifluoromethyl)-1,2,4-oxadiazol-3-yl]phenyl}methyl)-1H-pyrazole-4-carboxamide+SX, N-methoxy-N-methyl-1-({4-[5-(trifluoromethyl)-1,2,4-oxadiazol-3-yl]phenyl}methyl)-1H-pyrazole-4-carboxamide+SX, N,N-dimethyl-1-({4-[5-(trifluoromethyl)-1,2,4-oxadiazol-3-yl]phenyl}methyl)-1H-1,2,4-triazol-3-amine+SX, N-methyl-4-[5-(trifluoromethyl)-1,2,4-oxadiazol-3-yl]benzamide+SX, methyl 2-[2-chloro-4-(4-chlorophenoxy)phenyl]-2-hydroxy-3-(1H-1,2,4-triazol-1-yl)propanoate+SX, ethyl 2-[2-chloro-4-(4-chlorophenoxy)phenyl]-2-hydroxy-3-(1H-1,2,4-triazol-1-yl)propanoate+SX, methyl 2-[2-(trifluoromethyl)-4-(4-chlorophenoxy)phenyl]-2-hydroxy-3-(1H-1,2,4-triazol-1-yl)propanoate+SX, 1-(2,3-dimethylpyridin-5-yl)-4,4-difluoro-3,3-dimethyl-3,4-dihydroisoquinoline+SX, 1-[2-(difluoromethyl)-3-methylpyridin-5-yl]-4,4-difluoro-3,3-dimethyl-3,4-dihydroisoquinoline+SX, 2,2-difluoro-N-[6-({[1-(1-methyl-1H-tetrazol-5-yl)benzimidazo]-2-yl]oxy}methyl)pyridin-2-yl]-2-phenoxyacetamide+SX, 1-[2-(1-chlorocyclopropyl)-3-(3-chloro-2-fluorophenyl)-2-hydroxypropyl]-1H-imidazole-5-carbonitrile+SX, ethyl 1-[(4-{[2-(trifluoromethyl)-1,3-dioxolan-2-yl]methoxy}phenyl)methyl]-1H-pyrazole-4-carboxylate+SX, ethyl 1-[(4-{[(1Z)-2-ethoxy-3,3,3-trifluoro-1-propen-1-yl]oxy}phenyl)methyl]-1H-pyrazole-4-carboxylate+SX, 6-chloro-3-(3-cyclopropyl-2-fluorophenoxy)-N-[2-(2,4-dimethylphenyl)-2,2-difluoroethyl]-5-methylpyridazine-4-carboxamide+SX, 6-chloro-3-(3-cyclopropyl-2-fluorophenoxy)-N-[2-(3,4-dimethylphenyl)-2,2-difluoroethyl]-5-methylpyridazine-4-carboxamide+SX, 6-chloro-N-[2-(2-chloro-4-methylphenyl)-2,2-difluoroethyl]-3-(3-cyclopropyl-2-fluorophenoxy)-5-methylpyridazine-4-carboxamide+SX, 2-[cyano(2,6-difluoropyridin-4-yl)amino]-N-(2,2-dimethylcyclobutyl)-5-methylthiazole-4-carboxamide+SX, 2-[cyano(2,6-difluoropyridin-4-yl)amino]-N-(spiro[3.4]octan-1-yl)-5-methylthiazole-4-carboxamide+SX, 2-[cyano(2,6-difluoropyridin-4-yl)amino]-N-hexyl-5-methylthiazole-4-carboxamide+SX, 2-[acetyl (2,6-difluoropyridin-4-yl)amino]-N-(2,2-dimethylcyclobutyl)-5-methylthiazole-4-carboxamide+SX, 2-[(2-methoxyacetyl) (2,6-difluoropyridin-4-yl)amino]-N-(2,2-dimethylcyclobutyl)-5-methylthiazole-4-carboxamide+SX, 2-[(2-methylpropanoyl) (2,6-difluoropyridin-4-yl)amino]-N-(2,2-dimethylcyclobutyl)-5-methylthiazole-4-carboxamide+SX, 2-[(2,6-difluoropyridin-4-yl)amino]-N-(2,2-dimethylcyclobutyl)-5-methylthiazole-4-carboxamide+SX, 2-{[(oxetan-3-yl)carbonyl](2,6-difluoropyridin-4-yl)amino}-N-(2,2-dimethylcyclobutyl)-5-methylthiazole-4-carboxamide+SX, 2-{[(oxolan-3-yl)carbonyl](2,6-difluoropyridin-4-yl)amino}-N-(2,2-dimethylcyclobutyl)-5-methylthiazole-4-carboxamide+SX, 2-{[(oxan-4-yl)carbonyl](2,6-difluoropyridin-4-yl)amino}-N-(2,2-dimethylcyclobutyl)-5-methylthiazole-4-carboxamide+SX, 2-[(2,6-difluoropyridin-4-yl)amino]-N-(2,2-dimethylcyclobutyl)-5-methylthiazole-4-carboxamide+SX, 2-{[(oxetan-3-yl)carbonyl](2,6-difluoropyridin-4-yl)amino}-N-(2,2-dimethylcyclobutyl)-5-methylthiazole-4-carboxamide+SX, 2-{[(oxolan-3-yl)carbonyl](2,6-difluoropyridin-4-yl)amino}-N-(2,2-dimethylcyclobutyl)-5-methylthiazole-4-carboxamide+SX, 2-{[(oxan-4-yl)(carbonyl](2,6-difluoropyridin-4-yl)aamino}-N-(2,2-dimethylcyclobutyl)-5-methylthiazole-4-carboxamide+SX, 5-[5-(difluoromethyl)-1,3,4-oxadiazol-2-yl]-N-[1-(2-fluorophenyl)ethyl]pyrimidin-2-amine+SX, 5-[5-(difluoromethyl)-1,3,4-oxadiazol-2-yl]-N-[1-(2,6-difluorophenyl)ethyl]pyrimidin-2-amine+SX, 5-[5-(difluoromethyl)-1,3,4-oxadiazol-2-yl]-N-[1-(3,5-difluorophenyl)ethyl]pyrimidin-2-amine+SX, 5-[5-(difluoromethyl)-1,3,4-oxadiazol-2-yl]-N-[1-(6-chloropyridin-3-yl)ethyl]pyrimidin-2-amine+SX, 5-[5-(difluoromethyl)-1,3,4-oxadiazol-2-yl]-N-[1-(2-fluorophenyl)cyclopropyl]pyrimidin-2-amine+SX, 5-[5-(difluoromethyl)-1,3,4-oxadiazol-2-yl]-N-[1-(2,6-difluorophenyl)cyclopropyl]pyrimidin-2-amine+SX, 5-[5-(difluoromethyl)-1,3,4-oxadiazol-2-yl]-N-[1-(2-fluoro-3-methoxyphenyl)cyclopropyl]pyrimidin-2-amine+SX, 5-[5-(difluoromethyl)-1,3,4-oxadiazol-2-yl]-2-{[1-(2,6-difluorophenyl)cyclopropyl]oxy}pyrimidine+SX, 3-[3-(3-cyclopropyl-2-fluorophenoxy)-6-methylpyridazin-4-yl]-5-[(2,4-dimethylphenyl)methyl]-5,6-dihydro-4H-1,2,4-oxadiazine+SX, (5S)-3-[3-(3-cyclopropyl-2-fluorophenoxy)-6-methylpyridazin-4-yl]-5-[(2,4-dimethylphenyl)methyl]-5,6-dihydro-4H-1,2,4-oxadiazine+SX, 3-[3-(3-chloro-2-fluorophenoxy)-6-methylpyridazin-4-yl]-5-[(4-bromo-2-methylphenyl)methyl]-5,6-dihydro-4H-1,2,4-oxadiazine+SX, 3-[3-(3-chloro-2-fluorophenoxy)-6-methylpyridazin-4-yl]-5-[(2-chloro-4-methylphenyl)methyl]-5,6-dihydro-4H-1,2,4-oxadiazine+SX, (5S)-3-[3-(3-chloro-2-fluorophenoxy)-6-methylpyridazin-4-yl]-5-[(2-chloro-4-methylphenyl)methyl]-5,6-dihydro-4H-1,2,4-oxadiazine+SX, (5R)-3-[3-(3-chloro-2-fluorophenoxy)-6-methylpyridazin-4-yl]-5-[(2-chloro-4-methylphenyl)methyl]-5,6-dihydro-4H-1,2,4-oxadiazine+SX, N-((2S)-1-{3-[2-(5-fluoro-2-methoxyphenyl)-2-hydroxyethyl]-5-[(E)-1-(isopropoxyimino)ethyl]-2,6-dioxo-3,6-dihydropyrimidin-1(2H)-yl}-3-methylbutan-2-yl)-2-methylpropanamide+SX, N-((2S)-1-{3-[2-(5-fluoro-2-methoxyphenyl)-2-hydroxyethyl]-5-[(E)-1-(isopropoxyimino)ethyl]-2,6-dioxo-3,6-dihydropyrimidin-1(2H)-yl}-3-methylbutan-2-yl)-2,2-dimethylpropanamide+SX, N-((2S)-1-{3-[2-(5-fluoro-2-methoxyphenyl)-2-hydroxyethyl]-5-[(E)-1-(isopropoxyimino)ethyl]-2,6-dioxo-3,6-dihydropyrimidin-1(2H)-yl}propan-2-yl)-2-methylpropanamide+SX, N-((2S)-1-{3-[2-(2-methoxyphenyl)-2-hydroxyethyl]-5-[(E)-1-(isopropoxyimino)ethyl]-2,6-dioxo-3,6-dihydropyrimidin-1(2H)-yl}propan-2-yl)-2-methylpropanamide+SX, N-((2S)-1-{3-[2-(5-fluoro-2-methoxyphenyl)-2-(2-cyanoethoxy)ethyl]-5-[1-(isopropoxyimino)ethyl]-2,6-dioxo-3,6-dihydropyrimidin-1(2H)-yl}propan-2-yl)-2-methylpropanamide+SX, methyl ({5-[1-(2,6-difluoro-4-isopropylphenyl)-1H-pyrazol-3-yl]-2-methylphenyl}methyl)carbamate+SX, methyl ({5-[1-(2,6-difluoro-4-cyclopropylphenyl)-1H-pyrazol-3-yl]-2-methylphenyl}methyl)carbamate+SX, methyl ({5-[1-(2,6-difluoro-4-methoxyphenyl)-1H-pyrazol-3-yl]-2-methylphenyl}methyl)carbamate+SX, methyl (Z)-2-(5-cyclopentyl-2-methylphenoxy)-3-methoxyprop-2-enoate+SX, methyl (Z)-2-(5-cyclohexyl-2-methylphenoxy)-3-methoxyprop-2-enoate+SX, methyl (Z)-2-[(3-isopropyl-1H-pyrazol-1-yl)-2-methylphenoxy]-3-methoxyprop-2-enoate+SX, 1-(4,5-dimethyl-1H-benzimidazol-1-yl)-4,4-difluoro-3,3-dimethyl-3,4-dihydroisoquinoline+SX, 1-(4,5-dimethyl-1H-benzimidazol-1-yl)-4,4,5-trifluoro-3,3-dimethyl-3,4-dihydroisoquinoline+SX, 1-(pyrazolo[1,5-a]pyridin-3-yl)-4,4-difluoro-3,3-dimethyl-3,4-dihydroisoquinoline+SX, 1-(6,7-dimethylpyrazolo[1,5-a]pyridin-3-yl)-6-fluoro-3,3-dimethylisoquinolin-4(3H)-one+SX, methyl (2Z)-3-methoxy-2-[(4-methyl[1,1′-biphenyl]-3-yl)oxy]prop-2-enoate+SX, Agrobacterium radiobactor strain K84+SX, Bacillus amyloliquefaciens strain PTA-4838 (Aveo(registered trademark) EZ Nematicide)+SX, Bacillus amyloliquefaciens strain AT332+SX, Bacillus amyloliquefaciens strain B3+SX, Bacillus amyloliquefaciens strain D747+SX, Bacillus amyloliquefaciens strain DB101+SX, Bacillus amyloliquefaciens strain DB102+SX, Bacillus amyloliquefaciens strain GB03+SX, Bacillus amyloliquefaciens strain FZB24+SX, Bacillus amyloliquefaciens strain FZB42+SX, Bacillus amyloliquefaciens strain IN937a+SX, Bacillus amyloliquefaciens strain MBI600+SX, Bacillus amyloliquefaciens strain QST713+SX, Bacillus amyloliquefaciens isolate strain B246+SX, Bacillus amyloliquefaciens strain F727+SX, Bacillus amyloliquefaciens subsp. plantarum strain D747+SX, Bacillus licheniformis strain HB-2+SX, Bacillus licheniformis strain SB3086+SX, Bacillus pumilus strain AQ717+SX, Bacillus pumilus strain BUF-33+SX, Bacillus pumilus strain GB34+SX, Bacillus pumilus strain QST2808+SX, Bacillus simplex strain CGF2856+SX, Bacillus subtilis strain AQ153+SX, Bacillus subtilis strain AQ743+SX, Bacillus subtilis strain BU1814+SX, Bacillus subtilis strain D747+SX, Bacillus subtilis strain DB101+SX, Bacillus subtilis strain FZB24+SX, Bacillus subtilis strain GB03+SX, Bacillus subtilis strain HAI0404+SX, Bacillus subtilis strain IAB/BSO3+SX, Bacillus subtilis strain MBI600+SX, Bacillus subtilis strain QST30002/AQ30002+SX, Bacillus subtilis strain QST30004/AQ30004+SX, Bacillus subtilis strain QST713+SX, Bacillus subtilis strain QST714+SX, Bacillus subtilis var. Amyloliquefaciens strain FZB24+SX, Bacillus subtilis strain Y1336+SX, Burkholderia cepacia+SX, Burkholderia cepacia type Wisconsin strain J82+SX, Burkholderia cepacia type Wisconsin strain M54+SX, Candida oleophila strain 0+SX, Candida saitoana+SX, Chaetomium cupreum+SX, Clonostachys rosea+SX, Coniothyrium minitans strain CGMCC8325+SX, Coniothyrium minitans strain CON/M/91-8+SX, cryptococcus albidus+SX, Erwinia carotovora subsp. carotovora strain CGE234M403+SX, Fusarium oxysporum strain Fo47+SX, Gliocladium catenulatum strain J1446+SX, Paenibacillus polymyxa strain AC-1+SX, Paenibacillus polymyxa strain BS-0105+SX, Pantoea agglomerans strain E325+SX, Phlebiopsis gigantea strain VRA1992+SX, Pseudomonas aureofaciens strain TX-1+SX, Pseudomonas chlororaphis strain 63-28+SX, Pseudomonas chlororaphis strain AFS009+SX, Pseudomonas chlororaphis strain MA342+SX, Pseudomonas fluorescens strain 1629RS+SX, Pseudomonas fluorescens strain A506+SX, Pseudomonas fluorescens strain CL145A+SX, Pseudomonas fluorescens strain G7090+SX, Pseudomonas sp. strain CAB-02+SX, Pseudomonas syringae strain 742RS+SX, Pseudomonas syringae strain MA-4+SX, Pseudozyma flocculosa strain PF-A22UL+SX, Pseudomonas rhodesiae strain HAI-0804+SX, Pythium oligandrum strain DV74+SX, Pythium oligandrum strain M1+SX, Streptomyces griseoviridis strain K61+SX, Streptomyces lydicus strain WYCD108US+SX, Streptomyces lydicus strain WYEC108+SX, Talaromyces flavus strain SAY-Y-94-01+SX, Talaromyces flavus strain V117b+SX, Trichoderma asperellum strain ICC012+SX, Trichoderma asperellum SKT-1+SX, Trichoderma asperellum strain T25+SX, Trichoderma asperellum strain T34+SX, Trichoderma asperellum strain TV1+SX, Trichoderma atroviride strain CNCM 1-1237+SX, Trichoderma atroviride strain LC52+SX, Trichoderma atroviride strain IMI 206040+SX, Trichoderma atroviride strain SC1+SX, Trichoderma atroviride strain SKT-1+SX, Trichoderma atroviride strain T11+SX, Trichoderma gamsii strain ICC080+SX, Trichoderma harzianum strain 21+SX, Trichoderma harzianum strain DB104+SX, Trichoderma harzianum strain DSM 14944+SX, Trichoderma harzianum strain ESALQ-1303+SX, Trichoderma harzianum strain ESALQ-1306+SX, Trichoderma harzianum strain IIHR—Th-2+SX, Trichoderma harzianum strain ITEM908+SX, Trichoderma harzianum strain kd+SX, Trichoderma harzianum strain MO1+SX, Trichoderma harzianum strain SF+SX, Trichoderma harzianum strain T22+SX, Trichoderma harzianum strain T39+SX, Trichoderma harzianum strain T78+SX, Trichoderma harzianum strain TH35+SX, Trichoderma polysporum strain IMI206039+SX, trichoderma stromaticum+SX, Trichoderma virens strain G-41+SX, Trichoderma virens strain GL-21+SX, Trichoderma viride+SX, Variovorax paradoxus strain CGF4526+SX, Harpin protein+SX.

Combination of the Present ingredient in the above Group (c) and the Compound W of the present invention: 1-methylcyclopropene+SX, 1,3-diphenylurea+SX, 2,3,5-triiodobenzoic acid+SX, IAA ((1H-indol-3-yl)acetic acid)+SX, IBA (4-(1H-indol-3-yl)butyric acid)+SX, MCPA (2-(4-chloro-2-methylphenoxy) acetic acid)+SX, MCPB (4-(4-chloro-2-methylphenoxy)butyric acid)+SX, 4-CPA (4-chlorophenoxyacetic acid)+SX, 5-aminolevulinic acid hydrochloride+SX, 6-benzylaminopurine+SX, abscisic acid+SX, AVG (aminoethoxyvinylglycine)+SX, anisiflupurin+SX, ancymidol+SX, butralin+SX, calcium carbonate+SX, calcium chloride+SX, calcium formate+SX, calcium peroxide+SX, calcium polysulfide+SX, calcium sulfate+SX, chlormequat-chloride+SX, chlorpropham+SX, choline chloride+SX, cloprop+SX, cyanamide+SX, cyclanilide+SX, daminozide+SX, decan-1-ol+SX, dichlorprop+SX, dikegulac+SX, dimethipin+SX, diquat+SX, ethephon+SX, ethychlozate+SX, flumetralin+SX, flurprimidol+SX, forchlorfenuron+SX, formononetin+SX, Gibberellin A+SX, Gibberellin A3+SX, inabenfide+SX, Kinetin+SX, lipochitooligosaccharide SP104+SX, maleic hydrazide+SX, mefluidide+SX, mepiquat-chloride+SX, oxidized glutathione+SX, paclobutrazol+SX, pendimethalin+SX, prohexadione-calcium+SX, prohydrojasmon+SX, pyraflufen-ethyl+SX, sintofen+SX, sodium 1-naphthaleneacetate+SX, sodium cyanate+SX, thidiazuron+SX, triapenthenol+SX, tribufos+SX, trinexapac-ethyl+SX, uniconazole-P+SX, 2-(naphthalen-1-yl)acetamide+SX, [4-oxo-4-(2-phenylethyl)amino]butylate+SX, methyl 5-(trifluoromethyl)benzo[b]thiophene-2-carboxylate+SX, 3-[(6-chloro-4-phenylquinazolin-2-yl)amino]propan-1-ol+SX, Claroideoglomus etunicatum+SX, Claroideoglomus claroideum+SX, Funneliformis mosseae+SX, Gigaspora margarita+SX, Gigaspora rosea+SX, Glomus aggregatum+SX, Glomus deserticola+SX, Glomus monosporum+SX, Paraglomus brasillianum+SX, Rhizophagus clarus+SX, Rhizophagus intraradices RTI-801+SX, Rhizophagus irregularis DAOM 197198+SX, Azorhizobium caulinodans+SX, Azospirillum amazonense+SX, Azospirillum brasilense XOH+SX, Azospirillum brasilense Ab-V5+SX, Azospirillum brasilense Ab-V6+SX, Azospirillum caulinodans+SX, Azospirillum halopraeferens+SX, Azospirillum irakense+SX, Azospirillum lipoferum+SX, Bradyrhizobium elkanii SEMIA 587+SX, Bradyrhizobium elkanii SEMIA 5019+SX, Bradyrhizobium japonicum TA-11+SX, Bradyrhizobium japonicum USDA 110+SX, Bradyrhizobium liaoningense+SX, Bradyrhizobium lupini+SX, Delftia acidovorans RAY209+SX, Mesorhizobium ciceri+SX, Mesorhizobium huakii+SX, Mesorhizobium loti+SX, Rhizobium etli+SX, Rhizobium galegae+SX, Rhizobium leguminosarum bv. Phaseoli+SX, Rhizobium leguminosarum bv. Trifolii+SX, Rhizobium leguminosarum bv. Viciae+SX, Rhizobium trifolii+SX, Rhizobium tropici+SX, Sinorhizobium fredii+SX, Sinorhizobium meliloti+SX, Zucchini Yellow Mosaik Virus weak strain+SX.

Combination of the Present ingredient in the above Group (d) and the Compound W of the present invention: anthraquinone+SX, deet+SX, icaridin+SX.

The ratio of the Compound W of the present invention to the Present ingredient includes, but not limited thereto, as a ratio by weight (the Compound W of the present invention: the Present ingredient) 1,000:1 to 1:1,000, 500:1 to 1:500, 100:1 to 1:100, 50:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, and 1:50, and the others.

The Compound W of the present invention has control effect on harmful arthropods such as harmful insects and harmful mites, harmful nematodes, and harmful mollusks. Examples of the harmful arthropods, harmful nematodes, and harmful mollusks include the followings.

Hemiptera:
from the family Delphacidae, for example, small brown planthopper (Laodelphax striatellus), brown planthopper (Nilaparvata lugens), white-backed planthopper (Sogatella furcifera), corn planthopper (Peregrinus maidis), cereal leafhopper (Javesella pellucida), sugarcane leafhopper (Perkinsiella saccharicida), white-bellied planthopper (Stenocranus pacificus) and Tagosodes orizicolus;
from the family Cicadellidae, for example, green rice leafhopper (Nephotettix cincticeps), green paddy leafhopper (Nephotettix virescens), rice leafhopper (Nephotettix nigropictus), zigzag-striped leafhopper (Recilia dorsalis), tea green leafhopper (Empoasca onukii), potato leafhopper (Empoasca fabae), corn leafhopper (Dalbulus maidis), rice leafhopper (Cofana spectra), and Amrasca biguttula biguttula;
from the family Aphrophoridae, for example, European spittlebug (Philaenus spumarius);
from the family Cercopidae, for example, Mahanarva posticata and Mahanarva fimbriolata;
from the family Aphididae, for example, bean aphid (Aphis fabae), soybean aphid (Aphis glycines), cotton aphid (Aphis gossypii), green apple aphid (Aphis pomi), apple aphid (Aphis spiraecola), green peach aphid (Myzus persicae), leaf-curling plum aphid (Brachycaudus helichrysi), cabbage aphid (Brevicoryne brassicae), rosy apple aphid(Dysaphis plantaginea), false cabbage aphid (Lipaphis erysimi), potato aphid (Macrosiphum euphorbiae), foxglove aphid (Aulacorthum solani), lettuce aphid (Nasonovia ribisnigri), grain aphid (Rhopalosiphum padi), corn aphid (Rhopalosiphum maidis), brown citrus aphid (Toxoptera citricida), mealy plum aphid (Hyalopterus pruni), cane aphid (Melanaphis sacchari), black rice root aphid (Tetraneura nigriabdominalis), sugarcane cottony aphid (Ceratovacuna lanigera), apple woolly aphid (Eriosoma lanigerum), and English grain aphid (Sitobion avenae);
from the family Phylloxeridae, for example, grapevine phylloxera (Daktulosphaira vitifoliae), Pecan phylloxera (Phylloxera devastatrix), Pecan leaf phylloxera (Phylloxera notabilis), and Southern pecan leaf phylloxera (Phylloxera russelae);
from the family Adelgidae, for example, hemlock woolly aphid (Adelges tsugae), balsam woolly aphid (Adelges piceae), and Aphrastasia pectinatae;
from the family Pentatomidae, for example, black rice bug (Scotinophara lurida), black paddy bug (Scotinophara coarctata), common green stink bug (Nezara antennata), white-spotted spined bug (Eysarcoris aeneus), lewis spined bug (Eysarcoris lewisi), white-spotted bug (Eysarcoris ventralis), Eysarcoris annamita, brown marmorated stink bug (Halyomorpha halys), green plant bug (Nezara viridula), brown stink bug(Euschistus heros), red banded stink bug(Piezodorus guildinii), legume stink bug (Piezodorus hybneri), Oebalus pugnax, and Dichelops melacanthus; from the family Cydnidae, for example, Scaptocoris castanea; from the family Alydidae, for example, bean bug (Riptortus clavatus), corbett rice bug (Leptocorisa chinensis), and rice bug (Leptocorisa acuta);
from the family Coreidae, for example, Cletus punctiger and Australian leaf-footed bug (Leptoglossus australis);
from the family Lygaeidae, for example, oriental chinch bug (Cavelerius saccharivorus), seed bug (Togo hemipterus), and chinch bug (Blissus leucopterus);
from the family Miridae, for example, rice leaf bug (Trigonotylus caelestialium), sorghum plant bug (Stenotus rubrovittatus), wheat leaf bug (Stenodema calcarata), and American tarnished plant bug (Lygus lineolaris);
from the family Aleyrodidae, for example, greenhouse whitefly (Trialeurodes vaporariorum), tobacco whitefly (Bemisia tabaci), citrus whitefly (Dialeurodes citri), citrus spiny whitefly (Aleurocanthus spiniferus), tea spiny whitefly (Aleurocanthus camelliae), and Pealius euryae;
from the family Diaspididae, for example, Abgrallaspis cyanophylli, red scale (Aonidiella aurantii), San Jose scale (Diaspidiotus perniciosus), white peach scale (Pseudaulacaspis pentagona), arrowhead scale (Unaspis yanonensis), and citrus snow scale (Unaspis citri);
from the family Coccidae, for example, pink wax scale (Ceroplastes rubens);
from the family Margarodidae, for example, fluted scale (Icerya purchasi) and seychelles fluted scale (Icerya seychellarum);
from the family Pseudococcidae, for example, solanum mealybug (Phenacoccus solani), cotton mealybug (Phenacoccus solenopsis), Japanese mealybug (Planococcus kraunhiae), white peach scale (Pseudococcus comstocki), citrus mealybug (Planococcus citri), currant mealybug (Pseudococcus calceolariae), long-tailed mealybug (Pseudococcus longispinus), and tuttle mealybug (Brevennia rehi);
from the family Psyllidae, for example, citrus psylla (Diaphorina citri), two-spotted citrus psyllid (Trioza erytreae), pear sucker (Cacopsylla pyrisuga), Cacopsylla chinensis, potato psyllid (Bactericera cockerelli), and Pear psylla (Cacopsylla pyricola);
from the family Tingidae, for example, sycamore lace bug (Corythucha ciliata), aster tingid (Corythucha marmorata), Japanese pear lace bug (Stephanitis nashi), and azalea lace bug (Stephanitis pyrioides);
from the family Cimicidae, for example, common bed bug (Cimex lectularius) and tropical bed bug (Cimex hemipterus);
from the family Cicadidae, for example, Quesada gigas;
from the family Reduviidae, for example, Triatoma infestans, large kissing bug (Triatoma rubrofasciata), Triatoma dimidiata, and Rhodonius prolixus;
and the others.

Lepidoptera:
from the family Crambidae, for example, rice stem borer (Chilo suppressalis), Dark-headed stem borer (Chilo polychrysus), white stem borer (Scirpophaga innotata), yellow paddy borer (Scirpophaga incertulas), Rupela albina, rice leaf roller (Cnaphalocrocis medinalis), Marasmia patnalis, rice leaf roller (Marasmia exigua), cotton leaf roller (Notarcha derogata), corn borer (Ostrinia furnacalis), European corn borer (Ostrinia nubilalis), cabbage webworm (Hellula undalis), grape leafroller (Herpetogramma luctuosale), bluegrass webworm (Parapediasia teterrellus), rice case-worm (Nymphula depunctalis), Sugarcane borer (Diatraea saccharalis), and eggplant fruit borer (Leucinodes orbonalis);
from the family Pyralidae, for example, lesser cornstalk borer (Elasmopalpus lignosellus), mealworm moth (Plodia interpunctella), persimmon bark borer (Euzophera batangensis), and fig moth (Cadra cautella);
from the family Noctuidae, for example, common cutworm (Spodoptera litura), beet armyworm (Spodoptera exigua), rice armyworm (Mythimna separata), cabbage moth (Mamestra brassicae), pink borer (Sesamia inferens), grass armyworm (Spodoptera mauritia), green rice caterpillar (Naranga aenescens), Spodoptera frugiperda, true armyworm (Spodoptera exempta), semitropical armyworm (Spodoptera eridania), black cutworm (Agrotis ipsilon), turnip moth (Agrotis segetum), beet worm (Autographa nigrisigna), rice looper (Plusia festucae), Soybean looper (Chrysodeixis includens), Trichoplusia spp., Heliothis spp. (such as tobacco budworm (Heliothis virescens)), Helicoverpa spp. (such as tobacco budworm (Helicoverpa armigera), corn earworm (Helicoverpa zea)), velvet-bean caterpillar (Anticarsia gemmatalis), cotton leafworm (Alabama argillacea), and hop vine borer (Hydraecia immanis);
from the family Pieridae, for example, common cabbage worm (Pieris rapae);
from the family Tortricidae, for example, oriental fruit moth (Grapholita molesta), Grapholita dimorpha, soybean moth (Leguminivora glycinivorella), Matsumuraeses azukivora, summer fruit tortrix (Adoxophyes orana fasciata), smaller tea tortrix (Adoxophyes honmai), Japanese tea tortrix (Homona magnanima), apple tortrix (Archips fuscocupreanus), codling moth (Cydia pomonella), sugarcane shoot borer (Tetramoera schistaceana), bean shoot borer (Epinotia aporema), citrus fruit borer (Citripestis sagittiferella), and European grapevine moth (Lobesia botrana);
from the family Gracillariidae, for example, tea leaf roller (Caloptilia theivora) and Asiatic apple leaf miner (Phyllonorycter ringoniella);
from the family Carposinidae, for example, peach fruit moth (Carposina sasakii);
from the family Lyonetiidae, for example, coffee leaf miner (Leucoptera coffeella), peach leaf miner (Lyonetia clerkella), and Lyonetia prunifoliella;
from the family Lymantriidae, for example, Lymantria spp. (such as gypsy moth (Lymantria dispar)) and Euproctis spp. (such as tea lymantriid (Euproctis pseudoconspersa));
from the family Plutellidae, for example, diamondback moth (Plutella xylostella);
from the family Gelechiidae, for example, peach worm (Anarsia lineatella), sweetpotato leaf folder (Helcystogramma triannulella), pink bollworm (Pectinophora gossypiella), potato moth (Phthorimaea operculella), and tomato leafminer (Tuta absoluta);
from the family Arctiidae, for example, American white moth (Hyphantria cunea);
from the family Castniidae, for example, giant sugarcane borer (Telchin licus);
from the family Cossidae, for example, Cossus insularis;
from the family Geometridae, for example, Ascotis selenaria;
from the family Limacodidae, for example, blue-striped nettle grub (Parasa lepida);
from the family Stathmopodidae, for example, persimmon fruit moth (Stathmopoda masinissa);
from the family Sphingidae, for example, tobacco hornworm (Acherontia lachesis);
from the family Sesiidae, for example, Nokona feralis, cherry borer (Synanthedon hector), and Synanthedon tenuis:
from the family Hesperiidae, for example, rice skipper (Parnara guttata);
from the family Tineidae, for example, casemaking clothes moth (Tinea translucens), common clothes moth (Tineola bisselliella);
and the others.

Thysanoptera:
from the family Thripidae, for example, western flower thrips (Frankliniella occidentalis), oriental thrips (Thrips palmi), yellow tea thrips (Scirtothrips dorsalis), onion thrips (Thrips tabaci), eastern flower thrips (Frankliniella intonsa), rice thrips (Stenchaetothrips biformis), Echinothrips americanus, and avocado thrips (Scirtothrips perseae);
from the family Phlaeothripidae, for example, aculeated rice thrips (Haplothrips aculeatus);
and the others.

Diptera:
from the family Anthomyiidae, for example, seedcorn maggot (Delia platura), onion maggot (Delia antiqua), and beet leaf miner (Pegomya cunicularia);
from the family Ulidiidae, for example, sugarbeet root maggot (Tetanops myopaeformis);
from the family Agromyzidae, for example, rice leaf miner (Agromyza oryzae), tomato leaf miner (Liriomyza sativae), chrysanthemum leaf miner (Liriomyza trifolii), and pea leafminer (Chromatomyia horticola);
from the family Chloropidae, for example, rice stem maggot (Chlorops oryzae);
from the family Tephritidae, for example, melon fly (Bactrocera cucurbitae), oriental fruit fly (Bactrocera dorsalis), Malaysian fruit fly (Bactrocera latifrons), olive fruit fly (Bactrocera oleae), Queensland fruit fly (Bactrocera tryoni), Mediterranean fruit fly (Ceratitis capitata), apple maggot (Rhagoletis pomonella), and Japanese cherry fruit fly (Rhacochlaena japonica);
from the family Ephydridae, for example, smaller rice leaf miner (Hydrellia griseola), whorl maggot (Hydrellia philippina), and paddy stem maggot (Hydrellia sasakii);
from the family Drosophilidae, for example, cherry drosophila (Drosophila suzukii), and common fruit fly (Drosophila melanogaster);
from the family Phoridae, for example, Megaselia spiracularis;
from the family Psychodidae, for example, Clogmia albipunctata;
from the family Sciaridae, for example, Bradysia difformis and Bradysia odoriphaga;
from the family Cecidomyiidae, for example, hessian fly (Mayetiola destructor) and paddy gall fly (Orseolia oryzae);
from the family Diopsidae, for example, Diopsis macrophthalma;
from the family Glossinidae, for example, Glossina palpalis and Glossina morsitans;
from the family Simuliidae, for example, Simulium japonicum and Simulium damnosum;
from the subfamily Phlebotominae;
from the family Tipulidae, for example, rice crane fly (Tipula aino), common cranefly (Tipula oleracea), and European cranefly (Tipula paludosa);
from the family Culicidae, for example, common house mosquito (Culex pipiens pallens), Culex tritaeniorhynchus,Culex pipiens f. molestus, brown house mosquito (Culex quinquefasciatus), northern house mosquito (Culex pipiens pipiens), Culex vishnui, Asian tiger mosquito (Aedes albopictus), dengue mosquito (Aedes aegypti), Chinese malaria mosquito (Anopheles sinensis), Anopheles gambiae, Anopheles stephensi, Anopheles coluzzii, Anopheles albimanus, Anopheles sundaicus, Anopheles arabiensis, Anopheles funestus, Anopheles darlingi, Anopheles farauti, and Anopheles minimus;
from the family Simulidae, for example, Prosimulium yezoensis and Simulium ornatum;
from the family Tabanidae, for example, Tabanus trigonus;
from the family Muscidae, for example, house fly (Musca domestica), false stable fly (Muscina stabulans), biting house fly (Stomoxys calcitrans), and buffalo fly (Haematobia irritans);
from the family Calliphoridae;
from the family Sarcophagidae;
from the family Chironomidae, for example, Chironomus plumosus, Chironomus yoshimatsui, and Glyptotendipes tokunagai;
from the family Fannidae;
and the others.

Coleoptera:
from the family Chrysomelidae, for example, Diabrotica spp. (such as western corn rootworm (Diabrotica virgifera virgifera), southern corn rootworm (Diabrotica undecimpunctata howardi), northern corn rootworm (Diabrotica barberi), Mexican corn rootworm (Diabrotica virgifera zeae), banded cucumber beetle (Diabrotica balteata), cucurbit beetle(Diabrotica speciosa)), bean leaf beetle (Cerotoma trifurcata), barley leaf beetle (Oulema melanopus), cucurbit leaf beetle (Aulacophora femoralis), striped flea beetle (Phyllotreta striolata), cabbage flea beetle (Phyllotreta cruciferae), western black flea beetle (Phyllotreta pusilla), cabbage stem flea beetle (Psylliodes chrysocephala), hop flea beetle (Psylliodes punctulata), Colorado potato beetle (Leptinotarsa decemlineata), rice leaf beetle (Oulema oryzae), grape colaspis (Colaspis brunnea), corn flea beetle (Chaetocnema pulicaria), sweet-potato flea beetle (Chaetocnema confinis), potato flea beetle (Epitrix cucumeris), rice leaf beetle (Dicladispa armigera), southern corn leaf beetle (Myochrous denticollis), Laccoptera quadrimaculata, tobacco flea beetle (Epitrix hirtipennis), daikon leaf beetle (Phaedon brassicae) and two-striped leaf beetle (Medythia nigrobilineata);
from the family Carabidae, for example, Seedcorn beetle (Stenolophus lecontei) and slender seed-corn ground beetle (Clivina impressifrons);
from the family Scarabaeidae, for example, cupreus chafer (Anomala cuprea), soybean beetle (Anomala rufocuprea), Anomala albopilosa, Japanese beetle (Popillia japonica), yellowish elongate chafer (Heptophylla picea), European Chafer(Rhizotrogus majalis), Tomarus gibbosus, Holotrichia spp., Phyllophaga spp. (such as June beetle (Phyllophaga crinita)), and Diloboderus spp. (such as Diloboderus abderus);
from the family Anthriibidae, for example, coffee bean weevil (Araecerus coffeae);
from the family Aponidae, for example, sweet-potato weevil (Cylas formicarius);
from the family Bruchidae, for example, Mexican bean weevil (Zabrotes subfasciatus);
from the family Scolytidae, for example, pine beetle (Tomicus piniperda) and coffee berry borer (Hypothenemus hampei);
from the family Curculionidae, for example, West Indian sweet-potato weevil (Euscepes postfasciatus), alfalfa weevil (Hypera postica), maize wevil (Sitophilus zeamais), rice weevil (Sitophilus oryzae), grain weevil (Sitophilus granarius), rice plant weevil (Echinocnemus squameus), rice water weevil (Lissorhoptrus oryzophilus), Rhabdoscelus lineaticollis, boll weevil (Anthonomus grandis), nunting billbug (Sphenophorus venatus), southern corn billbug (Sphenophorus callosus), soybean stalk weevil (Sternechus subsignatus), sugarcane weevil (Sphenophorus levis), rusty gourd-shaped weevil (Scepticus griseus), brown gourd-shaped weevil (Scepticus uniformis), Aracanthus spp. (such as Aracanthus mourei), and cotton root borer (Eutinobothrus brasiliensis);
from the family Tenebrionidae, for example, red meal beetle (Tribolium castaneum), mason beetle (Tribolium confusum), and lesser mealworm (Alphitobius diaperinus);
from the family Coccinellidae, for example, twenty-eight-spotted ladybird (Epilachna vigintioctopunctata);
from the family Bostrychidae, for example, common powder-post beetle (Lyctus brunneus), and lesser grain borer (Rhizopertha dominica);
from the family Ptinidae;
from the family Cerambycidae, for example, citrus long-horned beetle (Anoplophora malasiaca), Migdolus fryanus, and peach borer (Aromia bungii);
from the family Elateridae, for example, Melanotus okinawensis, barley wireworm (Agriotes fuscicollis), Melanotus legatus, Anchastus spp., Conoderus spp., Ctenicera spp., Limonius spp., and Aeolus spp.;
from the family Staphylinidae, for example, Paederus fuscipes;
from the family Dermestidae, for example, varied carpet beetle (Anthrenus verbasci), hide beetle (Dermestes maculates), and khapra beetle (Trogoderma granarium);
from the family Anobiidae, for example, tobacco beetle (Lasioderma serricorne), and biscuit beetle (Stegobium paniceum);
from the family Laemophloeidae, for example, flat grain beetle (Cryptolestes ferrugineus);
from the family Silvanidae, for example, saw-toothed grain beetle (Oryzaephilus surinamensis);
from the family Nitidulidae, for example, blossom beetle (Brassicogethes aeneus);
and the others.

Orthoptera:
from the family Acrididae, for example, oriental migratory locust (Locusta migratoria), Moroccan locust (Dociostaurus maroccanus), Australian plague locust (Chortoicetes terminifera), red locust (Nomadacris septemfasciata), brown locust(Locustana pardalina), tree locust (Anacridium melanorhodon), Italian locust (Calliptamus italicus), differential grasshopper (Melanoplus differentialis), two-striped grasshopper (Melanoplus bivittatus), migratory grasshopper (Melanoplus sanguinipes), red-legged grasshopper (Melanoplus femurrubrum), clear-winged grasshopper (Camnula pellucida), desert locust (Schistocerca gregaria), Yellow-winged locust(Gastrimargus musicus), spur-throated locust (Austracris guttulosa), Japanese grasshopper (Oxya yezoensis), rice grasshopper (Oxya japonica), and Bombay locust (Patanga succincta);
from the family Gryllotalpidae, for example, oriental mole cricket (Gryllotalpa orientalis);
from the family Gryllidae, for example, house cricket (Acheta domestica), and emma field cricket (Teleogryllus emma);
from the family Tettigoniidae, for example, mormon cricket (Anabrus simplex);
and the others.

Hymenoptera:
from the family Tenthredinidae, for example, beet sawfly (Athalia rosae) and nippon cabbage sawfly (Athalia japonica);
from the family Formicidae, for example, Solenopsis spp. (such as red imported fire ant (Solenopsis invicta), tropical fire ant (Solenopsis geminata)), Atta spp. (such as brown leaf-cutting ant (Atta capiguara), Acromyrmex spp., Paraponera clavata, black house ant (Ochetellus glaber), pharaoh ant (Monomorium pharaonis), Argentine ant (Linepithema humile), Formica japonica, Pristomyrmex punctutus, Pheidole noda, big-headed ant (Pheidole megacephala), Camponotus spp. (such as Camponotus japonicus, Camponotus obscuripes), Pogonomyrmex spp. (such as western harvester ant (Pogonomyrmex occidentalis)), Wasmania spp. (such as Wasmania auropunctata), and long-legged ant (Anoplolepis gracilipes);
from the family Vespidae, for example, Asian giant hornet (Vespa mandarinia, Vespa simillima, Vespa analis, Asian hornet(Vespa velutina), and Polistes jokahamae;
from the family Siricidae, for example, pine wood wasp (Urocerus gigas);
from the family Bethylidae;
and the others.

Blattodea:
from the family Ectobiidae, for example, German cockroach (Blattella germanica);
from the family Blattidae, for example, smoky-brown cockroach (Periplaneta fuliginosa), American cockroach (Periplaneta americana), Australian cockroach (Periplaneta australasiae), brown cockroach (Periplaneta brunnea), and black cockroach (Blatta orientalis);
from the family Termitidae, for example, Japanese termite (Reticulitermes speratus), Formosan termite (Coptotermes formosanus), western drywood termite (Incisitermes minor), Cryptotermes domesticus, Odontotermes formosanus, Neotermes koshunensis, Glyptotermes satsumensis, Glyptotermes nakajimai, Glyptotermes fuscus, Hodotermopsis sjostedti, Coptotermes guangzhouensis, Reticulitermes amamianus, Reticulitermes miyatakei, Reticulitermes kanmonensis, Nasutitermes takasagoensis, Pericapritermes nitobei, Sinocapritermes mushae, and Cornitermes cumulans;
and the others.

Siphonaptera:
from the family Pulicidae, for example, human flea (Pulex irritans), cat flea (Ctenocephalides felis), dog flea (Ctenocephalides canis), oriental rat flea (Xenopsylla cheopis), and chicken flea (Echidnophaga gallinacea); from the family Pulicidae, for example, chigoe flea (Tunga penetrans);
from the family Ceratophyllidae, for example, European rat flea (Nosopsyllus fasciatus); and the others.

Psocodae:
from the family Pediculidae, for example, head louse (Pediculus humanus capitis);
from the family Pthiridae, for example, crab louse (Pthirus pubis);
from the family Haematopinidae, for example, short-nosed cattle louse (Haematopinus eurysternus) and pig louse (Haematopinus suis);
from the family Linognathidae, for example, blue cattle louse (Linognathus vituli), sheep face louse (Linognathus ovillus), and capillate louse (Solenopotes capillatus);
from the family Bovicoliidae, for example, cattle biting louse (Bovicola bovis), sheep biting louse (Bovicola ovis), Bovicola breviceps, Damalinia forficula, and Werneckiella spp.;
from the family Trichodectidae, for example, dog biting louse (Trichodectes canis) and cat louse (Felicola subrostratus);
from the family Menoponidae, for example, common chicken louse (Menopon gallinae), chicken body louse (Menacanthus stramineus), and Trinoton spp;
from the family Trimenoponidae, for example, Cummingsia spp.;
from the family Trogiidae, for example, death watch (Trogium pulsatorium);
from the family Liposcelidae or Liposcelididae, for example, book louse (Liposcelis corrodens), Liposcelis bostrychophila, Liposcelis pearmani and Liposcelis entomophila;
and the other.

Thysanura:
from the family Lepismatidae, for example, oriental silverfish (Ctenolepisma villosa) and moth fish (Lepisma saccharina);
and the others.

Acari:
from the family Tetranychidae, for example, common red spider mite (Tetranychus urticae), kanzawa spider mite (Tetranychus kanzawai), red spider mite (Tetranychus evansi), citrus red mite (Panonychus citri), fruit-tree red spider mite (Panonychus ulmi), and Oligonychus spp.;
from the family Eriophyidae, for example, Japanese citrus rust mite (Aculops pelekassi), Phyllocoptruta citri, tomato mite (Aculops lycopersici), purple mite (Calacarus carinatus), tea rust mite (Acaphylla theavagrans), Eriophyes chibaensis, apple bud mite (Aculus schlechtendali), Aceria diospyri, Aceria tosichella, and Shevtchenkella sp.;
from the family Tarsonemidae, for example, broad mite (Polyphagotarsonemus latus);
from the family Tenuipalpidae, for example, Brevipalpus phoenicis;
from the family Tuckerellidae;
from the family Ixodidae, for example, Haemaphysalis longicornis, Haemaphysalis flava, Haemaphysalis japonica, Haemaphysalis campanulata, American dog tick (Dermacentor variabilis), Dermacentor taiwanensis, Rocky Mountain wood tick (Dermacentor andersoni), netted tick (Dermacentor reticulatus), Ixodes ovatus, Ixodes persulcatus, black-legged tick (Ixodes scapularis), Ixodes pacificus, Ixodes holocyclus, Ixodes ricinus, lone star tick (Amblyomma americanum), gulf coast tick (Amblyomma maculatum), Rhipicephalus microplus, cattle tick (Rhipicephalus annulatus), brown dog tick (Rhipicephalus sanguineus), Rhipicephalus appendiculatus, and Rhipicephalus decoloratus;
from the family Argasidae, for example, fowl tick (Argas persicus), Ornithodoros hermsi, and Ornithodoros turicata;
from the family Acaridae, for example, cereal mite (Tyrophagus putrescentiae) and grassland mite (Tyrophagus similis);
from the family Pyroglyphidae, for example, American house dust mite (Dermatophagoides farinae) and European house dust mite (Dermatophagoides pteronyssinus);
from the family Cheyletidae, for example, Cheyletus eruditus, Cheyletus malaccensis, Chelacaropsis moorei, and Cheyletiella yasguri;
from the family Psoroptidae, for example, sheep scab mite (Psoroptes ovis), horse psoroptic mange mite (Psoroptes equi), Knemidocoptes mutans, ear mange mite (Otodectes cynotis), and Chorioptes spp.;
from the family Sarcoptidae, for example, Notoedres cati, Notoedres muris, and itch mite (Sarcoptes scabiei);
from the family Listrophoridae, for example, Listrophorus gibbus;
from the family Dermanyssidae, for example, bird mite (Dermanyssus gallinae);
from the family Macronyssidae, for example, feather mite (Ornithonyssus sylviarum) and tropical rat mite (Ornithonyssus bacoti);
from the family Varroidae, for example, Varroa jacobsoni;
from the family Demodicidae, for example, dog follicle mite (Demodex canis) and cat follicle mite (Demodex cati);
from the family Trombiculidae, for example, Leptotrombidium akamushi, Leptotrombidium pallidum, and Leptotrombidium scutellare;
and the others.

Araneae:
from the family Eutichuridae, for example, Cheiracanthium japonicum;
from the family Theridiidae, for example, red-back spider (Latrodectus hasseltii);
and the others

Polydesmida:
from the family Paradoxosomatidae, for example, flat-backed millipede (Oxidus gracilis) and Nedyopus tambanus;
and the others.

Isopoda:
from the family Armadillidiidae, common pill bug (Armadillidium vulgare);
and the others.

Chilopoda:
from the family Scutigeridae, for example, Thereuonema hilgendorfi;
from the family Scolopendridae, giant tropical centipede (Scolopendra subspinipes);
from the family Ethopolyidae, Bothropolys rugosus;
and the others.

Gastropoda:
from the family Limacidae, for example, tree slug (Limax marginatus) and garden tawny slug (Limax flavus); from the family Philomycidae, for example, Meghimatium bilineatum;
from the family Ampullariidae, for example, golden apple snail (Pomacea canaliculata);
from the family Lymnaeidae, for example, Austropeplea ollula;
and the others.

Nematoda:
from the family Aphelenchoididae, for example, rice white-tip nematode (Aphelenchoides besseyi);
from the family Pratylenchidae, for example, root lesion nematode (Pratylenchus coffeae), Pratylenchus brachyurus, California meadow nematode (Pratylenchus neglectus), and Radopholus similis;
from the family Heteroderidae, for example, javanese root-knot nematode (Meloidogyne javanica), southern root-knot nematode (Meloidogyne incognita), guava root-knot nematodes (Meloidogyne enterolobii), northern root-knot nematode (Meloidogyne hapla), Columbia root-knot nematode (Meloidogyne chitwoodi), soybean cyst nematode (Heterodera glycines), potato cyst nematode (Globodera rostochiensis), and white potato cyst nematode (Globodera pallida);
from the family Hoplolaimidae, for example, Rotylenchulus reniformis;
from the family Anguinidae, for example, strawberry bud nematode (Nothotylenchus acris), and stem nematode (Ditylenchus dipsaci);
from the family Tylenchulidae, for example, citrus nematode (Tylenchulus semipenetrans);
from the family Longidoridae, for example, dagger nematode (Xiphinema index);
from the family Trichodoridae;
from the family Parasitaphelenchidae, for example, pine wilt disease (Bursaphelenchus xylophilus);
and the others.

The harmful arthropods such as harmful insects and harmful mites, harmful mollusks and harmful nematodes may be those having a reduced susceptibility to or a developed resistance to an insecticide, a mitecide, a molluscicide or a nematicide.

The method for controlling harmful arthropods of the present invention is carried out by applying an effective amount of the Compound of the present invention, or the Compound W of the present invention or the Composition A to a harmful arthropod directly and/or a habitat thereof (for example, plant bodies, soil, an interior of a house, animal bodies). Examples of the method for controlling harmful arthropods of the present invention include foliage treatment, soil treatment, root treatment, shower treatment, smoking treatment, water surface treatment and seed treatment.

The Compound of the present invention, the Compound W of the present invention or the Composition A is usually used by mixing it with inert carrier(s) such as solid carrier(s), liquid carrier(s), and gaseous carrier(s), surfactant(s), and the like, and as needed, adding thereto auxiliary agent(s) for formulation such as binder(s), dispersant(s), and stabilizer(s) to be formulated into an aqueous suspension formulation, an oily suspension formulation, an oil solution, an emulsifiable concentrate, an emulsion formulation, a microemulsion formulation, a microcapsule formulation, a wettable powder, a granular wettable powder, a dust formulation, a granule, a tablet, an aerosol formulation, a resin formulation, or the like. In addition to these formulations, the Compound of the present invention, the Compound W of the present invention or the Composition A may be used by formulating it into a dosage form described in Manual on development and use of FAO and WHO Specifications for pesticides, FAO Plant Production and Protection Papers-271-276, prepared by the FAO/WHO Joint Meeting on Pesticide Specifications, 2016, ISSN: 0259-2517.

These formulations usually comprise 0.0001 to 99% by weight ratio of the Compound of the present invention, the Compound W of the present invention or the Composition A.

Examples of the solid carrier include fine powders and granules of clays (for example, pyrophyllite clay and kaolin clay), talc, calcium carbonate, diatomaceous earth, zeolite, bentonite, acid white clay, attapulgite, white carbon, ammonium sulfate, vermiculite, perlite, pumice, silica sand, chemical fertilizers (for example, ammonium sulfate, ammonium phosphate, ammonium nitrate, urea, and ammonium chloride), and the others; as well as resins (for example, polyethylene, polypropylene, polyester, polyurethane, polyamide, and polyvinyl chloride).

Examples of the liquid carrier include water, alcohols (for example, ethanol, cyclohexanol, benzyl alcohol, propylene glycol, and polyethylene glycol), ketones (for example, acetone and cyclohexanone), aromatic hydrocarbons (for example, xylene, phenyl xylyl ethane, and methylnaphthalene), aliphatic hydrocarbons (for example, hexane and cyclohexane), esters (for example, ethyl acetate, methyl oleate, and propylene carbonate), nitriles (for example, acetonitrile), ethers (for example, ethylene glycol dimethyl ether), amides (for example, N, N-dimethylformamide and N, N-dimethyloctanamide), sulfoxides (for example, dimethyl sulfoxide), lactams (for example, N-methylpyrrolidone and N-octylpyrrolidone), fatty acids (for example, oleic acid), and vegetable oils (for example, soybean oil).

Examples of the gaseous carrier include fluorocarbon, butane gas, LPG (liquefied petroleum gas), dimethyl ether, nitrogen, and carbon dioxide.

Examples of the surfactant include nonionic surfactants (for example, polyoxyethylene alkyl ethers, polyoxyethylene alkyl aryl ethers, and polyethylene glycol fatty acid esters), and anionic surfactants (for example, alkyl sulfonates, alkyl aryl sulfonates, and alkyl sulfates).

Examples of the other auxiliary agent for formulation include binders, dispersants, colorants, and stabilizers, and the specific examples thereof include polysaccharides (for example, starch, gum arabic, cellulose derivatives, and alginic acid), lignin derivatives, water-soluble synthetic polymers (for example, polyvinyl alcohol, polyvinyl pyrrolidone, and polyacrylic acids), acidic isopropyl phosphate, and dibutylhydroxytoluene.

Moreover, some adjuvants can be employed to improve or help the efficacy of the Compound W of the present invention. The specific examples thereof include Nimbus (registered trademark), Assist (registered trademark), Aureo (registered trademark), Iharol (registered trademark), Silwet L-77 (registered trademark), BreakThru (registered trademark), SundanceII (registered trademark), Induce (registered trademark), Penetrator (registered trademark), AgriDex (registered trademark), Lutensol A8 (registered trademark) NP-7 (registered trademark), Triton (registered trademark), Nufilm (registered trademark), Emulgator NP7 (registered trademark), Emulad (registered trademark), TRITON X 45 (registered trademark), AGRAL 90 (registered trademark) AGROTIN (registered trademark), ARPON (registered trademark), EnSpray N (registered trademark), and BANOLE (registered trademark).

As used herein, examples of the plant include whole plant, stem and leaf, flower, ear, fruit, tree stem, branch, crown, seed, vegetative reproductive organ, and seedling.

A vegetative reproduction organ means a part of plant such as root, stem, and leaf which has a growth capability even when said part is separated from the plant body and placed into soil. Examples of the vegetative reproduction organ include tuberous root, creeping root, bulb, corm or solid bulb, tuber, rhizome, stolon, rhizophore, cane cuttings, propagule, and vine cutting. Stolon is also referred to as “runner”, and propagule is also referred to as “propagulum” and categorized into broad bud and bulbil. Vine cutting means a shoot (collective term of leaf and stem) of sweet potato, glutinous yam, or the like. Bulb, corm or solid bulb, tuber, rhizome, cane cuttings, rhizophore, and tuberous root are also collectively referred to as “bulb”. For example, cultivation of potato starts with planting a tuber into soil, and the tuber to be used is generally referred to as “seed potato”.

Examples of a method for controlling harmful arthropods by applying an effective amount of the Compound of the present invention, the Compound W of the present invention or the Composition A to soils include a method of applying an effective amount of the Compound of the present invention, the Compound W of the present invention or the Composition A to soils before planting plants or after planting plants. Specifically, examples of the application method include planting hole treatment (spraying into planting holes, soil mixing after planting hole treatment), plant foot treatment (plant foot spraying, soil mixing after plant foot treatment, irrigation at plant foot, plant foot treatment at a later seeding raising stage), planting furrow treatment (planting furrow spraying, soil mixing after planting furrow treatment), planting row treatment (planting row spraying, soil mixing after planting row treatment, planting row spraying at a growing stage), planting row treatment at the time of sowing (planting row spraying at the time of sowing, soil mixing after planting row treatment at the time of sowing), broadcast treatment (overall soil surface spraying, soil mixing after broadcast treatment), side-article treatment, treatment of water surface (application to water surface, application to water surface after flooding), other soil spraying treatment (spraying of a granular formulation on leaves at a growing stage, spraying under a canopy or around a tree stem, spraying on the soil surface, mixing with surface soil, spraying into seed holes, spraying on the ground surfaces of furrows, spraying between plants), other irrigation treatment (soil irrigation, irrigation at a seedling raising stage, chemical solution injection treatment, irrigation of a plant part just above the ground, chemical solution drip irrigation, chemigation), seedling raising box treatment (spraying into a seedling raising box, irrigation of a seedling raising box, flooding into a seedling raising box with chemical solution), seedling raising tray treatment (spraying on a seedling raising tray, irrigation of a seedling raising tray, flooding into a seedling raising tray with chemical solution), seedbed treatment (spraying on a seedbed, irrigation of a seedbed, spraying on a lowland rice nursery, immersion of seedlings), seedbed soil incorporation treatment (mixing with seedbed soil, mixing with seedbed soil before sowing, spraying at sowing before covering with soils, spraying at sowing after covering with soils, mixing with covering with soils), and other treatment (mixing with culture soil, plowing under, mixing with surface soil, mixing with soil at the place where raindrops fall from a canopy, treatment at a planting position, spraying of a granule formulation on flower clusters, mixing with a paste fertilizer).

Examples of the application to seeds (or seed treatments) include an application of the Compound of the present invention, the Compound W of the present invention or the Composition A to seeds or vegetative reproductive organs, and specific examples thereof include spraying treatment in which a suspension of the Compound of the present invention, the Compound W of the present invention or the Composition A is sprayed onto seed surface or the vegetative reproductive organ surface in the form of mist; smearing treatment in which the Compound of the present invention, the Compound W of the present invention or the Composition A is coated a surface of seeds or the vegetative reproductive organ; a soaking treatment in which the seeds are soaked into the solution of the Compound of the present invention, the Compound W of the present invention or the Composition A for a certain time; and a method for coating the seeds or the vegetative reproductive organ with a carrier containing the Compound of the present invention, the Compound W of the present invention or the Composition A (film coating treatment, pellet coating treatment). Examples of the above-described vegetative reproductive organ include particularly seed potato.

When the Composition A is applied to seeds or vegetative reproductive organs, the Composition A may be also applied to seeds or vegetative reproductive organs as a single formulation, or the Composition A may be applied to seeds or vegetative reproductive organs as multiple different formulations by multiple times. Examples of the method in which the Composition A is applied as multiple different formulations by multiple times include, for example, a method in which the formulations comprising as an active component the Compound of the present invention or the Compound W of the present invention only are applied, and seeds or vegetative reproductive organs are air dried, followed by applying the formulations comprising the Present ingredient; and a method in which the formulations comprising as an active component the Compound of the present invention, the Compound W of the present invention and the Present ingredients are applied, and seeds or vegetative reproductive organs are air dried, followed by applying the formulations comprising the Present ingredients other than the already-applied Present ingredients, are included.

As used herein, seeds or vegetative reproductive organs carrying the Compound of the present invention, the Compound W of the present invention or the Composition A means seeds or vegetative reproductive organs in the state where the Compound of the present invention, the Compound W of the present invention or the Composition A is adhered to a surface of the seeds or the vegetative reproductive organ. The above-described seeds or vegetative reproductive organs carrying the Compound of the present invention, the Compound W of the present invention of the Composition A may be adhered by any other materials that are different from the Compound of the present invention, the Compound W of the present invention or the Composition A before or after being adhered the Compound of the present invention, the Compound W of the present invention or the Composition A to the seeds or vegetative reproductive organs.

Also, when the Composition A is adhered in a form of layer(s) to a surface of seeds or vegetative reproductive organ, the layer(s) is/are composed of one layer or a multiple layers. Also, when multiple layers are formed, each of the layer may be composed of a layer comprising one or more active ingredients, or a combination of a layer comprising one or more active ingredients and a layer not comprising an active ingredient.

Seeds or vegetative reproductive organs carrying the Compound of the present invention, the Compound W of the present invention or the Composition A can be obtained, for example, by applying the formulations comprising the Compound of the present invention, the Compound W of the present invention or the Composition A by the above-described application method to seeds to seeds or vegetative reproductive organs.

When the Compound of the present invention, the Compound W of the present invention or the Composition A is applied for harmful arthropods control in agricultural fields, the application dose thereof is usually within a range of 1 to 10,000 g of the Compound of the present invention or the Compound W of the present invention per 10,000 m². In the case of being applied to seeds or vegetative reproductive organs, the dose of application dose thereof is usually within a range of 0.001 to 100 g of the Compound of the present invention or the Compound W of the present invention per 1 Kg of seeds. When the Compound of the present invention, the Compound W of the present invention or the Composition A is formulated into an emulsifiable concentrate, a wettable powder or a flowable etc., they are usually applied by diluting them with water so as to make an effective concentration of the active ingredients 0.01 to 10,000 ppm, and the dust formulation or the granular formulation, etc., is usually applied as itself without diluting them.

Also, the resin preparation which is processed into a sheet or a string may be applied by winding a plant with a sheet or a string of the resin preparation, putting a string of the resin preparation around a crop so that the plant is surrounded by the string, or laying a sheet of the resin preparation on the soil surface near the root of a plant.

When the Compound of the present invention, the Compound W of the present invention or the Composition A is used to control harmful arthropods that live inside a house, the application dose as an amount of the Compound of the present invention or the Compound W of the present invention is usually within a range from 0.01 to 1,000 mg per 1 m² of an area to be treated, in the case of using it on a planar area. In the case of using it spatially, the application dose as an amount of the Compound of the present invention or the Compound W of the present invention is usually within a range from 0.01 to 500 mg per 1 m³ of the space to be treated. When the Compound of the present invention, the Compound W of the present invention or the Composition A is formulated into emulsifiable concentrates, wettable powders, flowables or the others, such formulations are usually applied after diluting it with water in such a way that a concentration of the active ingredient is within a range from 0.1 to 10,000 ppm. In the case of being formulated into oil solutions, aerosols, smoking agents, poison baits and the others, such formulations are used as itself without diluting it.

When the Compound of the present invention, the Compound W of the present invention or the Composition A is used for controlling external parasites of livestock such as cows, horses, pigs, sheep, goats and chickens and small animals such as dogs, cats, rats and mice, the composition of the present invention may be applied to the animals by a known method in the veterinary field. Specifically, when systemic control is intended, the composition of the present invention is administered to the animals as a tablet, a mixture with feed or a suppository, or by injection (including intramuscular, subcutaneous, intravenous and intraperitoneal injections). On the other hand, when non-systemic control is intended, the composition of the present invention is applied to the animals by means of spraying of the oil solution or aqueous solution, pour-on or spot-on treatment, or washing of the animal with a shampoo formulation, or by putting a collar or ear tag made of the resin formulations to the animal. In the case of being administered to an animal body, the dose of the compound of the Compound of the present invention, the Compound W of the present invention or the Composition A is usually within a range from 0.1 to 1,000 mg per 1 kg of an animal body weight.

Also, the composition of the Compound of the present invention, the Compound W of the present invention or the Composition A may be used as an agent for controlling harmful arthropods in agricultural lands such as paddy fields, fields, turfs, and orchards. Examples of the plants include the followings, corn (dent corn, flint corn, flour corn, popcorn, waxy corn, sweet corn, and field corn), rice (long grain rice, short grain rice, medium grain rice, japonica rice, tropical japonica rice, indica rice, javanica rice, paddy rice, upland rice, floating rice, direct-seeded rice, transplanted rice, and glutinous rice), wheat (bread wheat (hard wheat, soft wheat, medium wheat, red wheat, and white wheat), durum wheat, spelt wheat, and club wheat, winter wheat and spring wheat of them), barley (two-rowed barley (=barley for brewery), six-rowed barley, hull-less barley, and pearl barley, winter barley and spring barley of them), rye (winter rye and spring rye), triticale (winter triticale and spring triticale), oat (winter oat and spring oat), sorghum, cotton (upland cotton and Pima cotton), soybean (ripe seed harvest soybean, green soybeans, and early harvest soybeans, indeterminate type, determinate type, and semi-determinate type of them), peanut, buckwheat, beet (beets for sugar production, beets for feed, beets for root vegetable, beets for leaf vegetable, and beets for fuel), rapeseed (winter rapeseed and spring rapeseed), canola (winter canola and spring canola), sunflower (sunflowers for oil extraction, edible sunflowers, and sunflowers for ornamental purpose), sugar cane, tobacco, tea, mulberry, solanaceous vegetables (for example, eggplant, tomato, pimento, pepper, and potato), cucurbitaceous vegetables (for example, cucumber, pumpkin, zucchini, water melon, and melon), cruciferous vegetables (for example, Japanese radish, white turnip, horseradish, kohlrabi, Chinese cabbage, cabbage, leaf mustard, broccoli, and cauliflower), asteraceous vegetables (for example, burdock, crown daisy, artichoke, and lettuce), liliaceous vegetables (for example, welsh onion, onion, garlic, and asparagus), ammiaceous vegetables (for example, carrot, parsley, celery, and parsnip), chenopodiaceous vegetables (for example, spinach and Swiss chard), lamiaceous vegetables (for example, perilla, mint, and basil), strawberry, sweet potato, glutinous yam, eddoe, pomaceous fruits (for example, apple, pear, Japanese pear, Chinese white pear, Chinese quince, and quince), stone fleshy fruits (for example, peach, plum, nectarine, Japanese apricot (Prunus mume), cherry fruit, apricot, and prune), citrus fruits (for example, Citrus unshiu, orange, lemon, lime, and grapefruit), nuts (for example, chestnuts, walnuts, hazelnuts, almond, pistachio, cashew nuts, and macadamia nuts), berry fruits (for example, blueberry, cranberry, blackberry, and raspberry), grapes, Japanese persimmon, fig, olive, Japanese plum, banana, coffee, date palm, coconuts, ornamental plants, forest plants, turfs, grasses, and the others.

The above plants are not specifically limited as long as they are generally cultivated cultivars. The above plants also include plants which may be produced by natural breeding, plants which may be generated by mutation, F1 hybrid plants, and genetically modified crops. Examples of the genetically modified crops include plants which have resistance to HPPD (4-hydroxyphenylpyruvate dioxygenase enzyme) inhibitors such as isoxaflutole, ALS (acetolactate synthase) inhibitors such as imazethapyr and thifensulfuron-methyl, EPSP (5-enolpyruvylshikimate-3-phosphate synthase) inhibitors, glutamine synthetase inhibitors, PPO (protoporphyrinogen oxidase) inhibitors, or herbicide such as bromoxynil and dicamba; plants which can synthesize a selective toxin known in Bacillus spp, such as Bacillus thuringiensis or the like; and plants which can synthesize a gene fragment or the like which is partially identical to an endogenous gene derived from a harmful insect, and induce a gene silencing (RNAi; RNA interference) in the target harmful insect to achieve a specific insecticidal activity.

### EXAMPLES

Hereinafter, the present invention will be described in more detail with reference to production examples, formulation examples, test examples, and the like, but the present invention is not limited to these examples.

In the present specification, Me represents a methyl group, Et represents an ethyl group, Pr represents a propyl group, i-Pr represents an isopropyl group, Bu represents a butyl group, t-Bu represents a tert-butyl group, C₂F₅ represents a perfluoroethyl group, c-Pr represents a cyclopropyl group, c-Bu represents a cyclobutyl group, c-C₅H₉ represents a cyclopentyl group, c-C₆H₁₁ represents a cyclohexyl group, Ph represents a phenyl group, Py2 represents a 2-pyridyl group, Py3 represents a 3-pyridyl group, and Py4 represents a 4-pyridyl group. When c-Pr, Ph, Py2, Py3, and Py4 have a substituent, the substituent is described together with a substitution position before the symbol. For example, 1-CN-c-Pr represents a 1-cyanocyclopropyl group, 3-F-Ph represents a 3-fluorophenyl group, 3,4,5-(OCH₃)₃-Ph represents a 3,4,5-trimethoxyphenyl group, and 5-CF₃-Py₂ represents a 5-(trifluoromethyl)-2-pyridyl group.

First, production examples of the Compound W of the present invention and production intermediates thereof will be shown.

### Reference Production Example 1

7.8 g of (1-amino-2-methylpropane-2-yl)dimethylamine was added to 7.7 g of ethyl 5-(hydroxymethyl) isoxazole-3-carboxylate, and the mixture was stirred at 140° C. for 2 hours. The resulting mixture was concentrated under reduced pressure. The resulting residue was subjected to silica gel chromatography (chloroform:methanol=5:1) to provide 5.5 g of Intermediate 1 represented by the following formula.

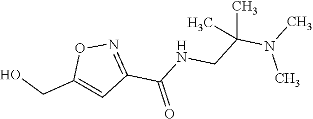 [SMILES: CN(C)C(C)(C)CNC(=O)c1cc(CO)on1]

Intermediate 1: ¹H-NMR (CDCl₃) δ: 7.56-7.41 (1H, m), 6.65 (1H, s), 4.79 (2H, s), 3.35 (2H, s), 2.23 (6H, s), 1.06 (6H, s).

### Reference Production Example 1-1

The compound produced in accordance with Reference production example 1 and physical properties thereof are Shown below.

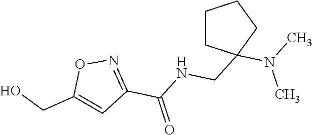 [SMILES: CN(C)C1(CNC(=O)c2cc(CO)on2)CCCC1]

Intermediate 2: ¹H-NMR (CDCl₃) δ: 7.57-7.40 (1H, m), 6.67 (1H, s), 4.80 (2H, s), 3.43 (2H, s), 2.24 (6H, s), 1.87-1.75 (2H, m), 1.71-1.56 (4H, m), 1.42-1.33 (2H, m).

### Reference Production Example 2

2.98 g of diethylphosphoryl chloride was added to a mixture of 2.77 g of Intermediate 1, 3.49 g of triethylamine, 0.14 g of 4-N, N-dimethylaminopyridine, and 30 mL of chloroform, and the mixture was stirred at room temperature for 1 hour. An aqueous sodium bicarbonate solution was added to the resulting mixture, which was then extracted with chloroform. The resulting organic layer was dried over anhydrous sodium sulfate and concentrated under reduced pressure. The resulting residue was subjected to silica gel chromatography (chloroform:methanol=10:1) to provide 2.79 g of Intermediate 3 represented by the following formula.

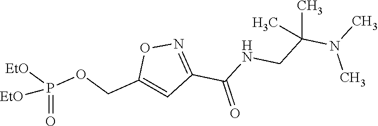 [SMILES: CCOP(=O)(OCC)OCc1cc(C(=O)NCC(C)(C)N(C)C)no1]

Intermediate 3: ¹H-NMR (CDCl₃) δ: 7.51-7.42 (1H, m), 6.80 (1H, s), 5.16 (2H, d), 4.21-4.09 (4H, m), 3.34 (2H, s), 2.22 (6H, s), 1.41-1.28 (6H, m), 1.05 (6H, s).

### Reference Production Example 2-1

The compound produced in accordance with Reference production example 2 and physical properties thereof are shown below.

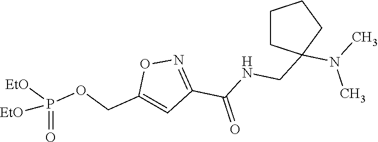 [SMILES: CCOP(=O)(OCC)OCc1cc(C(=O)NCC2(N(C)C)CCCC2)no1]

Intermediate 4: ¹H-NMR (CDCl₃) δ:7.51-7.42 (1H, m), 6.80 (1H, s), 5.16 (2H, d), 4.20-4.09 (4H, m), 3.43 (2H, s), 2.24 (6H, s), 1.87-1.76 (2H, m), 1.66-1.57 (4H, m), 1.42-1.30 (8H, m).

### Reference Production Example 3

14.2 g of cesium carbonate was added to a mixture of 4.93 g of 3,4,5-trimethylphenol, 8.56 g of 2,2-diethoxy-1-bromoethane, and 30 mL of DMF, and the mixture was stirred at 130° C. for 7 hours. Water was added to the resulting mixture, which was then extracted with methyl tert-butyl ether (hereinafter, referred to as MTBE). The resulting organic layer was dried over anhydrous magnesium sulfate and concentrated under reduced pressure to provide 9.13 g of Intermediate 5 represented by the following formula.

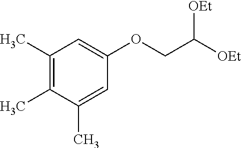 [SMILES: CCOC(COc1cc(C)c(C)c(C)c1)OCC]

Intermediate 5: ¹H-NMR (CDCl₃) δ: 6.61 (2H, s), 4.82 (1H, t), 3.97 (2H, d), 3.80-3.58 (4H, m), 2.24 (6H, s), 2.09 (3H, s), 1.22 (6H, t).

### Reference Production Example 4

13.7 g of a mixture of polyphosphoric acid:silica gel=1:1 was added to a mixture of 9.13 g of Intermediate 5 and 100 mL of toluene, and the mixture was stirred under heat-reflux for 4 hours. The resulting mixture was filtered and concentrated under reduced pressure. The resulting residue was subjected to silica gel chromatography (hexane) to provide 2.67 g of Intermediate 6 represented by the following formula.

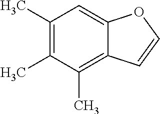

Intermediate 6: ¹H-NMR (CDCl₃) δ:7.51 (1H, d), 7.18 (1H, s), 6.73 (1H, d), 2.44 (3H, s), 2.39 (3H, s), 2.25 (3H, 3).

### Reference Production Example 5

7.57 mL of n-butyllithium (2.64 mol/L hexane solution) was added to a mixture of 2.67 g of Intermediate 6 and 30 mL of THF, and the mixture was stirred at −78° C. for 10 minutes. 4.34 g of 2-isopropoxy-4,4,5,5-tetramethyl-1, 3, 2-dioxaborolane was added to the resulting mixture, and the mixture was stirred at room temperature for 1 hour. An aqueous ammonium chloride solution was added to the resulting mixture, and the mixture was extracted with ethyl acetate. The resulting organic layer was dried over anhydrous sodium sulfate and concentrated under reduced pressure. The resulting residue was subjected to silica gel chromatography (hexane:ethyl acetate=5:1) to provide 4.13 g of Intermediate 7 represented by the following formula.

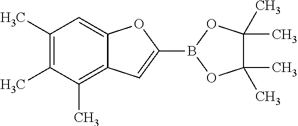

Intermediate 7: ¹H-NMR (CDCl₃) δ:7.42 (1H, s), 7.21 (1H, s), 2.44 (3H, s), 2.38 (3H, s), 2.23 (3H, s), 1.37 (12H, s).

### Reference Production Example 5-1

The compound produced in accordance with Reference production example 5 and physical properties thereof are shown below.

A compound represented by formula (A-1),

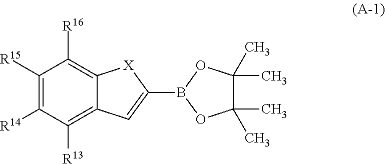

wherein the combination of R¹³, R¹⁴, R¹⁵, R¹⁶, and X is any one of the combinations shown in [Table A-1].

TABLE A-1

| Intermediate | R¹³ | R¹⁴ | R¹⁵ | R¹⁶ | X |
|---|---|---|---|---|---|
| 8 | H | H | Cl | Cl | O |
| 9 | H | OCF₃ | H | H | O |
| 10 | F | F | F | H | O |
| 11 | Cl | Cl | H | H | O |
| 12 | F | H | H | Cl | O |
| 13 | H | Me | Cl | H | O |
| 14 | H | Me | Me | H | O |
| 15 | Me | H | Me | H | O |
| 16 | Me | Cl | Me | H | O |
| 17 | H | C1 | Me | H | O |
| 18 | H | Me | OMe | H | O |
| 19 | Me | Me | H | H | S |

| | | | | |
|---|---|---|---|---|
| 20 | H | —OCH₂O— | H | O |

Intermediate 8: ¹H-NMR (CDCl₃) δ:7.44 (1H, d), 7.38 (1H, s), 7.33 (1H, d), 1.39 (12H, s).

Intermediate 9: ¹H-NMR (CDCl₃) δ: 7.55 (1H, d), 7.49 (1H, s), 7.38 (1H, s), 7.21 (1H, d), 1.40 (12H, s).

Intermediate 10: ¹H-NMR (CDCl₃) δ: 7.69 (1H, d), 6.85 (1H, d), 1.43 (12H, s).

Intermediate 11: ¹H-NMR (CDCl₃) δ: 7.48 (1H, s), 7.40 (2H, s), 1.40 (12H, s).

Intermediate 12: ¹H-NMR (CDCl₃) δ:7.49 (1H, s), 7.28 (1H, dd), 6.87 (1H, t), 1.39 (12H, s).

Intermediate 13: ¹H-NMR (CDCl₃) δ:7.56 (1H, s), 7.46 (1H, s), 7.30 (1H, s), 2.44 (3H, s), 1.39 (12H, s). Intermediate 14: ¹H-NMR (CDCl₃) δ:7.37 (1H, s), 7.33 (1H, s), 7.30 (1H, s), 2.36 (3H, s), 2.33 (3H, s), 1.38 (12H, s).

Intermediate 15: ¹H-NMR (CDCl₃) δ:7.42 (1H, s), 7.18 (1H, s), 6.86 (1H, s), 2.47 (3H, s), 2.43 (3H, s), 1.37 (12H, s).

Intermediate 16: ¹H-NMR (CDCl₃) δ:7.40 (1H, s), 7.28 (1H, s), 2.53 (3H, s), 2.48 (3H, s), 1.39 (12H, s).

Intermediate 17: ¹H-NMR (CDCl₃) δ:7.61 (1H, s), 7.41 (1H, s), 7.29 (1H, s), 2.47 (3H, s), 1.39 (12H, s).

Intermediate 18: ¹H-NMR (CDCl₃) δ:7.34 (1H, s), 7.29 (1H, s), 7.01 (1H, s), 3.85 (3H, s), 2.27 (3H, s), 1.38 (12H, s).

Intermediate 19: ¹H-NMR (CDCl₃) δ:7.97 (1H, s), 7.64 (1H, d), 7.19 (1H, d), 2.55 (3H, s), 2.39 (3H, s), 1.39 (12H, s).

Intermediate 20: ¹H-NMR (CDCl₃) δ:7.62 (1H, s), 7.02 (1H, s), 6.62 (1H, s), 6.06 (2H, s), 1.43 (12H, s).

### Reference Production Example 6

5.62 mL of n-butyllithium (2.64 mol/L hexane solution) was added to a mixture of 1.98 g of 5-isopropylbenzofuran and 15 mL of THF, and the mixture was stirred at −78° C. for 10 minutes. 3.40 mL of triisopropylborate was added to the resulting mixture, and the mixture was stirred at room temperature for 2 hours. Hydrogen chloride (2 mol/L aqueous solution) was added to the resulting mixture, and the mixture was extracted with ethyl acetate. The resulting organic layer was dried over anhydrous sodium sulfate and concentrated under reduced pressure to provide 2.37 g of Intermediate 21 represented by the following formula.

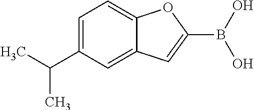

Intermediate 21: ¹H-NMR (CDCl₃) δ:7.59 (1H, s), 7.42 (1H, d), 7.17 (1H, d), 6.72 (1H, s), 3.07-2.96 (1H, m), 1.30 (6H, d).

### Reference Production Example 7

1.30 g of tetrakis(triphenylphosphine) palladium (0) was added to a mixture of 3.80 g of ethyl 5-[[(diethoxyphosphinyl)oxy] methyl]-3-isoxazole carboxylate produced by the method described in WO 2014/119696 A, 3.00 g of benzofuran-2-boronic acid, 4.66 g of potassium carbonate, 70 mL of toluene, and 20 mL of water, and the mixture was stirred under heat-reflux for 2 hours, Water was added to the resulting mixture, and the mixture was extracted with MTBE. The resulting organic layer was dried over anhydrous sodium sulfate and concentrated under reduced pressure. The resulting residue was subjected to to provide 1.33 g of Intermediate 22 represented by the following formula.

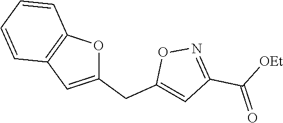 [SMILES: CCOC(=O)c1cc(Cc2cc3ccccc3o2)on1]

Intermediate 22: ¹H-NMR (CDCl₃) δ:7.52 (1H, d), 7.44 (1H, d), 7.35-7.19 (2H, m), 6.60 (1H, s), 6.56 (1H, s), 4.43 (2H, q), 4.37 (2H, s), 1.40 (3H, t).

### Reference Production Example 7-1

The compound produced in accordance with Reference production example 7 and physical properties thereof are shown below.

A compound represented by formula (A-2),

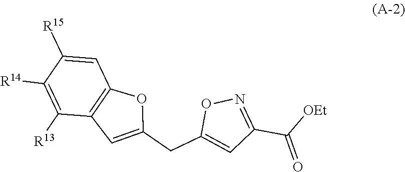

wherein the combination of R¹³, R¹⁴, R¹⁵, and X is any one of the combinations shown in [Table A-2].

TABLE A-2

| Intermediate | R¹³ | R¹⁴ | R¹⁵ | X |
|---|---|---|---|---|
| 23 | Cl | Cl | H | O |
| 24 | H | Me | H | O |
| 25 | H | Me | Me | O |
| 26 | H | H | H | S |
| 27 | H | H | Cl | S |
| 28 | H | F | H | O |
| 29 | H | H | F | O |
| 30 | H | H | Me | O |
| 31 | Me | H | Me | O |
| 32 | H | Me | Me | S |
| 33 | Me | Cl | Me | O |
| 34 | H | Cl | Me | O |
| 35 | H | Et | H | O |
| 36 | H | i-Pr | H | O |
| 37 | H | Cl | H | O |
| 38 | H | H | OMe | O |
| 39 | H | Me | OMe | O |
| 40 | H | Ph | H | O |
| 41 | Me | Me | H | S |
| 42 | H | Cl | Cl | O |
| 43 | Me | Me | Me | O |
| 44 | H | H | H | NMe |
| 45 | H | Me | Cl | O |
| 46 | F | F | F | O |

| | | | |
|---|---|---|---|
| 47 | H | —OCH₂O— | O |

Intermediate 23: ¹H-NMR (CDCl₃) δ: 7.35 (1H, d), 7.29 (1H, d), 6.69 (1H, s), 6.59 (1H, s), 4.44 (2H, q), 4.35 (2H, s), 1.41 (3H, t).

Intermediate 24: ¹H-NMR (CDCl₃) δ:7.32 (1H, d), 7.31 (1H, s), 7.08 (1H, s), 6.55 (1H, s), 6.52 (1H, s), 4.43 (2H, q), 4.31 (2H, s), 2.43 (3H, s), 1.40 (3H, t).

Intermediate 25: ¹H-NMR (CDCl₃) δ:7.27 (1H, s), 7.22 (1H, s), 6.52 (1H, s), 6.49 (1H, s), 4.42 (2H, q), 4.29 (2H, s), 2.35 (3H, s), 2.32 (3H, s), 1.40 (3H, t).

Intermediate 26: ¹H-NMR (CDCl₃) δ:7.73 (1H, d), 7.69 (1H, d), 7.37-7.29 (2H, m), 7.17 (1H, s), 6.52 (1H, s), 4.42 (2H, s), 4.42 (2H, q), 1.40 (3H, t).

Intermediate 27: ¹H-NMR (CDCl₃) δ:7.77 (1H, s), 7.62 (1H, d), 7.33 (1H, d), 7.13 (1H, s), 6.53 (1H, s), 4.43 (2H, q), 4.41 (2H, s), 1.40 (3H, t).

Intermediate 28: ¹H-NMR (CDCl₃) δ:7.36 (1H, dd), 7.18 (1H, dd), 7.00 (1H, td), 6.57 (2H, s), 4.44 (2H, q), 4.33 (2H, s), 1.41 (3H, t).

Intermediate 29: ¹H-NMR (CDCl₃) δ:7.44 (1H, dd), 7.17 (1H, dd), 7.00 (1H, td), 6.57 (1H, s), 6.56 (1H, s), 4.44 (2H, q), 4.32 (2H, s), 1.41 (3H, t).

Intermediate 30: ¹H-NMR (CDCl₃) δ:7.39 (1H, d), 7.25 (1H, d), 7.05 (1H, d), 6.54 (2H, s), 4.43 (2H, q), 4.30 (2H, s), 2.46 (3H, s), 1.40 (3H, t).

Intermediate 31: ¹H-NMR (CDCl₃) δ:7.08 (1H, s), 6.86 (1H, s), 6.54 (2H, s), 4.43 (2H, q), 4.30 (2H, s), 2.44 (3H, s), 2.42 (3H, s), 1.40 (3H, t).

Intermediate 32: ¹H-NMR (CDCl₃) δ:7.54 (1H, s), 7.48 (1H, s), 7.05 (1H, s), 6.49 (1H, s), 4.42 (2H, q), 4.38 (2H, s), 2.36 (3H, s), 2.35 (3H, s), 1.39 (3H, t).

Intermediate 33: ¹H-NMR (CDCl₃) δ:7.18 (1H, s), 6.55 (2H, s), 4.43 (2H, q), 4.31 (2H, s), 2.50 (3H, s), 2.47 (3H, s), 1.41 (3H, t).

Intermediate 34: ¹H-NMR (CDCl₃) δ:7.50 (1H, s), 7.31 (1H, s), 6.55 (1H, s), 6.51 (1H, s), 4.43 (2H, q), 4.31 (2H, s), 2.46 (3H, s), 1.41 (3H, t).

Intermediate 35: ¹H-NMR (CDCl₃) δ:7.35 (1H, d), 7.34 (1H, s), 7.12 (1H, d), 6.54 (2H, s), 4.43 (2H, q), 4.32 (2H, s), 2.73 (2H, q), 1.40 (3H, t), 1.27 (3H, t).

Intermediate 36: ¹H-NMR (CDCl₃) δ: 7.37 (1H, s), 7.36 (1H, d), 7.16 (1H, d), 6.55 (1H, s), 6.54 (1H, s), 4.43 (2H, q), 4.32 (2H, s), 3.06-2.94 (1H, m), 1.40 (3H, t), 1.29 (6H, d).

Intermediate 37: ¹H-NMR (CDCl₃) δ:7.50 (1H, d), 7.36 (1H, d), 7.24 (1H, dd), 6.57 (1H, s), 6.55 (1H, s), 4.44 (2H, q), 4.33 (2H, s), 1.41 (3H, t).

Intermediate 38: ¹H-NMR (CDCl₃) δ:7.38 (1H, d), 6.99 (1H, d), 6.86 (1H, dd), 6.55 (1H, s), 6.52 (1H, s), 4.43 (2H, g), 4.30 (2H, s), 3.85 (3H, s), 1.40 (3H, t).

Intermediate 39: ¹H-NMR (CDCl₃) δ:7.24 (1H, s), 6.93 (1H, s), 6.53 (1H, s), 6.47 (1H, s), 4.43 (2H, q), 4.28 (2H, s), 3.86 (3H, s), 2.27 (3H, s), 1.40 (3H, t).

Intermediate 40: ¹H-NMR (CDCl₃) δ:7.71 (1H, s), 7.63-7.32 (7H, m), 6.65 (1H, s), 6.58 (1H, s), 4.44 (2H, q), 4.36 (2H, s), 1.41 (3H, t).

Intermediate 41: ¹H-NMR (CDCl₃) δ:7.52 (1H, d), 7.23 (1H, s), 7.14 (1H, d), 6.51 (1H, s), 4.42 (2H, q), 4.42 (2H, s), 2.48 (3H, s), 2.38 (3H, s), 1.40 (3H, t).

Intermediate 42: ¹H-NMR (CDCl₃) δ:7.60 (1H, s), 7.56 (1H, s), 6.57 (1H, s), 6.54 (1H, s), 4.44 (2H, q), 4.33 (2H, s), 1.41 (3H, t).

Intermediate 43: ¹H-NMR (CDCl₃) δ:7.11 (1H, s), 6.54 (1H, s), 6.53 (1H, s), 4.42 (2H, g), 4.30 (2H, s), 2.40 (3H, s), 2.38 (3H, s), 2.23 (3H, s), 1.40 (3H, t).

Intermediate 44: ¹H-NMR (CDCl₃) δ: 7.58 (1H, d), 7.30 (1H, d), 7.23 (1H, t), 7.12 (1H, t), 6.42 (1H, s), 6.35 (1H, s), 4.41 (2H, q), 4.33 (2H, s), 3.66 (3H, s), 1.39 (3H, t).

Intermediate 45: ¹H-NMR (CDCl₃) δ:7.46 (1H, s), 7.36 (1H, s), 6.55 (1H, s), 6.51 (1H, s), 4.43 (2H, q), 4.31 (2H, s), 2.43 (3H, s), 1.41 (3H, t).

Intermediate 46: ¹H-NMR (CDCl₃) δ:7.65 (1H, d), 6.91 (1H, d), 6.40 (1H, s), 4.42 (2H, s), 4.41 (2H, q), 1.39 (3H, t).

Intermediate 47: ¹H-NMR (CDCl₃) δ:7.54 (1H, d), 6.92 (1H, s), 6.67 (1H, d), 6.39 (1H, s), 6.00 (2H, s), 4.40 (2H, q), 4.36 (2H, s), 1.38 (3H, t).

### Reference Production Example 8

4 mL of sodium hydroxide (1 mol/L aqueous solution) was added to a mixture of 1.33 g of Intermediate 22 and 4 mL of THF, and the mixture was stirred at room temperature for 30 minutes. 5 mL of hydrogen chloride (35% aqueous solution) was added to the resulting mixture under ice cooling, and the mixture was extracted with MTBE. The resulting organic layer was dried over anhydrous sodium sulfate and concentrated under reduced pressure to provide 1.26 g of Intermediate 48 represented by the following formula.

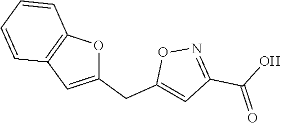 [SMILES: O=C(O)c1cc(Cc2cc3ccccc3o2)on1]

Intermediate 48: ¹H-NMR (CDCl₃) δ:7.53 (1H, d), 7.44 (1H, d), 7.32-7.18 (2H, m), 6.61 (1H, s), 6.61 (1H, s), 4.35 (2H, s).

### Reference Production Example 8-1

The compound produced in accordance with Reference production example 8 and physical properties thereof are shown below.

A compound represented by formula (A-3),

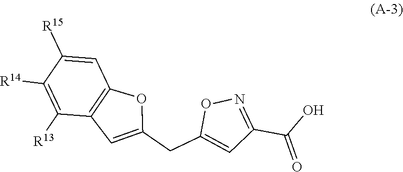

wherein the combination of R¹³, R¹⁴, R¹⁵, and X is any one of the combinations shown in [Table A-3].

TABLE A-3

| Intermediate | R¹³ | R¹⁴ | R¹⁵ | X |
|---|---|---|---|---|
| 49 | Cl | Cl | H | O |
| 50 | H | Me | H | O |
| 51 | H | Me | Me | O |
| 52 | H | H | H | S |
| 53 | H | H | Cl | S |
| 54 | H | F | H | O |
| 55 | H | H | F | O |
| 56 | H | H | Me | O |
| 57 | Me | H | Me | O |
| 58 | H | Me | Me | S |
| 59 | Me | Cl | Me | O |
| 60 | H | Cl | Me | O |
| 61 | H | Et | H | O |
| 62 | H | i-Pr | H | O |
| 63 | H | Cl | H | O |
| 64 | H | H | OMe | O |
| 65 | H | Me | OMe | O |
| 66 | H | Ph | H | O |
| 67 | Me | Me | H | S |

Intermediate 49: ¹H-NMR (CDCl₃) δ:7.73-7.41 (2H, m), 6.58 (1H, s), 6.51 (1H, s), 4.22 (2H, s), 4.17-4.03 (1H, m).

Intermediate 50: ¹H-NMR (CDCl₃) δ:7.35-7.29 (2H, m), 7.09 (1H, d), 6.61 (1H, s), 6.54 (1H, s), 4.34 (2H, s), 2.43 (3H, s).

Intermediate 51: ¹H-NMR (CDCl₃) δ: 7.28 (1H, s), 7.23 (1H, s), 6.59 (1H, s), 6.50 (1H, s), 4.31 (2H, s), 2.80-2.39 (1H, m), 2.35 (3H, s), 2.32 (3H, s).

Intermediate 52: ¹H-NMR (CDCl₃) δ:7.79 (1H, d), 7.72 (1H, d), 7.40-7.28 (2H, m), 7.18 (1H, s), 6.58 (1H, s), 4.45 (2H, s).

Intermediate 53: ¹H-NMR (DMSO-D₆) δ:8.09 (1H, d), 7.81 (1H, d), 7.39 (1H, d), 7.33 (1H, s), 6.63 (1H, s), 4.57 (2H, s).

Intermediate 54: ¹H-NMR (CDCl₃) δ:7.40-7.33 (1H, m), 7.22-7.15 (1H, m), 7.05-6.95 (1H, m), 6.63 (1H, s), 6.57 (1H, s), 4.35 (2H, s).

Intermediate 55: ¹H-NMR (CDCl₃) δ: 7.45 (1H, dd), 7.17 (1H, d), 7.00 (1H, t), 6.63 (1H, s), 6.59 (1H, s), 4.34 (2H, s).

Intermediate 56: ¹H-NMR (CDCl₃) δ:7.40 (1H, d), 7.26 (1H, s), 7.06 (1H, d), 6.61 (1H, s), 6.56 (1H, s), 4.36 (2H, s), 2.46 (3H, s).

Intermediate 57: ¹H-NMR (CDCl₃) δ:7.08 (1H, s), 6.87 (1H, s), 6.61 (1H, s), 6.56 (1H, s), 4.34 (2H, s), 2.44 (3H, s), 2.42 (3H, s).

Intermediate 58: ¹H-NMR (CDCl₃) δ:7.54 (1H, s), 7.48 (1H, s), 7.06 (1H, s), 6.56 (1H, s), 4.41 (2H, s), 2.36 (3H, s), 2.35 (3H, s).

Intermediate 59: ¹H-NMR (CDCl₃) δ:7.18 (1H, s), 6.60 (1H, s), 6.56 (1H, s), 4.30 (2H, s), 2.50 (3H, s), 2.47 (3H, s).

Intermediate 60: ¹H-NMR (CDCl₃) δ:7.51 (1H, s), 7.31 (1H, s), 6.62 (1H, s), 6.53 (1H, s), 4.33 (2H, s), 2.46 (3H, s).

Intermediate 61: ¹H-NMR (CDCl₃) δ:7.37-7.32 (2H, m), 7.12 (1H, d), 6.61 (1H, s), 6.56 (1H, s), 4.34 (2H, s), 2.73 (2H, q), 1.27 (3H, t).

Intermediate 62: ¹H-NMR (CDCl₃) δ:7.40-7.33 (2H, m), 7.16 (1H, d), 6.60 (1H, s), 6.57 (1H, s), 4.34 (2H, s), 3.06-2.94 (1H, m), 1.29 (6H, d).

Intermediate 63: ¹H-NMR (CDCl₃) δ:7.50 (1H, s), 7.36 (1H, d), 7.24 (1H, d), 6.62 (1H, s), 6.57 (1H, s), 4.80-4.72 (1H, m), 4.35 (2H, s).

Intermediate 64: ¹H-NMR (CDCl₃) δ:7.39 (1H, d), 6.99 (1H, s), 6.87 (1H, d), 6.61 (1H, s), 6.54 (1H, s), 4.32 (2H, s), 3.85 (3H, s), 1.25 (1H, s).

Intermediate 65: ¹H-NMR (CDCl₃) δ:7.24 (1H, s), 6.93 (1H, s), 6.59 (1H, s), 6.48 (1H, s), 4.31 (2H, s), 3.86 (3H, s), 2.27 (3H, s).

Intermediate 66: ¹H-NMR (CDCl₃) δ:7.75-7.32 (7H, m), 6.98 (1H, s), 6.66 (1H, s), 6.64 (1H, s), 4.38 (2H, s).

Intermediate 67: ¹H-NMR (CDCl₃) δ:7.53 (1H, d), 7.24 (1H, s), 7.14 (1H, d), 6.57 (1H, s), 4.44 (2H, s), 2.48 (3H, s), 2.39 (3H, s).

### Reference Production Example 9

0.70 g of 10% palladium/activated carbon (containing about 50% water) was added to a mixture of 6.96 g of 4-methyl-5-[(2-naphthalenylmethoxy)methyl]-N-[(tetrahydro-3-furanyl)methyl)]-3-isoxazole carboxamide produced by the method described in WO 2014/119696 A and 50 mL of ethanol, and the mixture was stirred at room temperature for 10 hours under a hydrogen atmosphere. The resulting mixture was filtered and concentrated under reduced pressure. The resulting residue was subjected to silica gel chromatography (hexane:ethyl acetate=1:1) to provide 2.45 g of Intermediate 68 represented by the following formula.

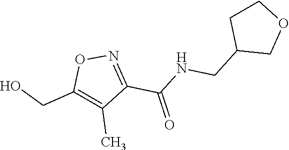 [SMILES: Cc1c(C(=O)NCC2CCOC2)noc1CO]

Intermediate 68: ¹H-NMR (CDCl₃) δ:7.19-7.09 (1H, m), 4.73 (2H, d), 3.95-3.89 (1H, m), 3.85-3.73 (2H, m), 3.61 (1H, dd), 3.52-3.35 (2H, m), 2.67-2.46 (2H, m), 2.27 (3H, s), 2.14-2.05 (1H, m), 1.73-1.66 (1H, m).

### Reference Production Example 10

3.08 g of triphenylphosphine was added to a mixture of 1.88 g of Intermediate 68, 3.89 g of carbon tetrabromide, and 50 mL of THF, and the mixture was stirred at room temperature for 15 hours. The resulting mixture was filtered and concentrated under reduced pressure. The resulting residue was subjected to silica gel. chromatography (hexane:ethyl acetate=2:1) to provide 1.59 g of Intermediate 69 represented by the following formula.

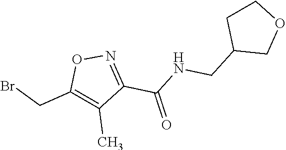 [SMILES: Cc1c(C(=O)NCC2CCOC2)noc1CBr]

Intermediate 69: ¹H-NMR (CDCl₃) δ:7.01-6.90 (1H, m), 4.46 (2H, s), 3.95-3.82 (2H, m), 3.77 (1H, dd), 3.59 (1H, dd), 3.50-3.38 (2H, m), 2.63-2.51 (1H, m), 2.26 (3H, s), 2.14-2.04 (1H, m), 1.72-1.62 (1H, m).

### Reference Production Example 11

0.50 g of tetrakis(triphenylphosphine) palladium (0) was added to a mixture of 1.32 g of Intermediate 69, 1.78 g of Intermediate 14, 1.81 g of potassium carbonate, 20 mL of toluene, and 8 mL of water, and the mixture was stirred under heat-reflux for 1 hour. Water was added to the resulting mixture, and the mixture was extracted with MTBE. The resulting organic layer was dried over anhydrous sodium sulfate and concentrated under reduced pressure. The resulting residue was subjected to silica gel chromatography (hexane:ethyl acetate=2:1) to provide 0.37 g of Intermediate 70 represented by the following formula.

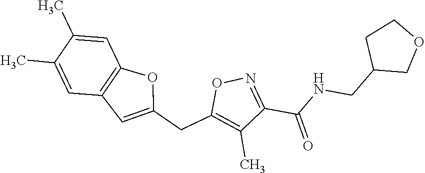 [SMILES: Cc1cc2cc(Cc3onc(C(=O)NCC4CCOC4)c3C)oc2cc1C]

Intermediate 70: ¹H-NMR (CDCl₃) δ:7.24 (1H, s), 7.20 (1H, s), 6.97-6.89 (1H, m), 6.39 (1H, s), 4.21 (2H, s), 3.94-3.82 (2H, m), 3.76 (1H, dd), 3.58 (1H, dd), 3.49-3.38 (2H, m), 2.61-2.51 (1H, m), 2.33 (3H, s), 2.31 (3H, s), 2.22 (3H, s), 2.13-2.05 (1H, m), 1.72-1.60 (1H, m).

### Reference Production Example 12

210 mg of potassium hydroxide was added to a mixture of 210 mg of Intermediate 70, 10 mL of ethanol, and 2 mL of water, and the mixture was stirred under heat-reflux for 4 hours and 30 minutes. Hydrogen chloride (1 mol/L aqueous solution) was added to the resulting mixture, and the mixture was extracted with MTBE. The resulting organic layer was dried over anhydrous sodium sulfate and concentrated under reduced pressure to provide 170 mg of Intermediate 71 represented by the following formula.

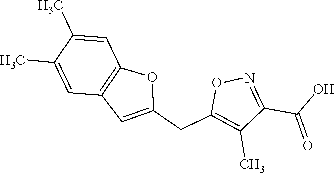 [SMILES: Cc1cc2cc(Cc3onc(C(=O)O)c3C)oc2cc1C]

Intermediate 71: ¹H-NMR (CDCl₃) δ:7.25 (1H, s), 7.21 (1H, s), 6.40 (1H, s), 2.92 (1H, dd), 2.85 (1H, dd), 2.34 (3H, s), 2.32 (3H, s), 1.89 (1H, d), 1.28 (3H, d).

### Reference Production Example 13

70 mg of bis(tricyclohexylphosphine) palladium (II) dichloride was added to a mixture of 290 mg of Intermediate 45, 200 mg of ethyl boronic acid, 580 mg of tripotassium phosphate, 5 mL of toluene, and 1 mL of water, and the mixture was stirred under heat-reflux for 3 hours and 30 minutes. Water was added to the resulting mixture, and the mixture was extracted with MTBE. The resulting organic layer was dried over anhydrous sodium sulfate and concentrated under reduced pressure. The resulting residue was subjected to silica gel chromatography (hexane:ethyl acetate=5:1) to provide 10 mg of Intermediate 72 represented by the following formula.

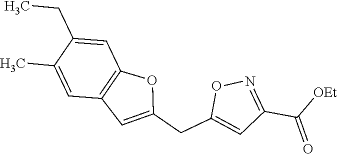 [SMILES: CCOC(=O)c1cc(Cc2cc3cc(C)c(CC)cc3o2)on1]

Intermediate 72: ¹H-NMR (CDCl₃) δ:7.28 (1H, s), 7.25 (1H, s), 6.53 (1H, s), 6.49 (1H, s), 4.43 (2H, q), 4.30 (2H, s), 2.70 (2H, q), 2.36 (3H, s), 1.40 (3H, s), 1.26 (3H, s).

### Reference Production Example 14

1.03 g of [1-(aminomethyl)cyclopentyl]methanol was added to a mixture of 1.80 g of Intermediate 51, 1.53 g of 1-ethyl-3-(3-dimethylaminopropyl) carbodiimide hydrochloride, 0.09 g of 1-hydroxybenzotriazole, and 10 mL of chloroform, and the mixture was stirred at room temperature for 16 hours. The resulting mixture was concentrated under reduced pressure. The resulting residue was subjected to silica gel chromatography (hexane:ethyl acetate=3:1) to provide 1.16 g of Intermediate 73 represented by the following formula.

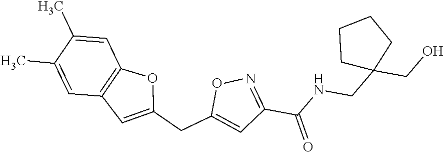 [SMILES: Cc1cc2cc(Cc3cc(C(=O)NCC4(CO)CCCC4)no3)oc2cc1C]

Intermediate 73: ¹H-NMR (CDCl₃) δ:7.28-7.22 (2H, m), 7.21 (1H, s), 6.58 (1H, s), 6.48 (1H, s), 4.28 (2H, s), 3.44 (1H, t), 3.40 (2H, d), 3.32 (2H, d), 2.35 (3H, s), 2.32 (3H, s), 1.69-1.58 (4H, m), 1.54-1.45 (2H, m), 1.42-1.33 (2H, m).

### Reference Production Example 15

910 mg of triphenylphosphine and 810 mg of dimethoxyethyl azodicarboxylate were added in this order to a mixture of 880 mg of Intermediate 73, 510 mg of phthalimide, and 20 mL of THF, and the mixture was stirred at room temperature for 3 hours. The resulting mixture was concentrated under reduced pressure. The resulting residue was subjected to silica gel chromatography (hexane:ethyl acetate=3:1) to provide 910 mg of Intermediate 74 represented by the following formula.

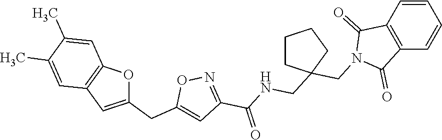 [SMILES: Cc1cc2cc(Cc3cc(C(=O)NCC4(CN5C(=O)c6ccccc6C5=O)CCCC4)no3)oc2cc1C]

Intermediate 74: ¹H-NMR (CDCl₃) δ:8.52-8.43 (1H, m), 7.93-7.85 (2H, m), 7.78-7.71 (2H, m), 7.26 (1H, s), 7.22 (1H, s), 6.60 (1H, s), 6.48 (1H, s), 4.30 (2H, s), 3.69 (2H, s), 3.28 (2H, d), 2.35 (3H, s), 2.32 (3H, s), 1.85-1.65 (4H, m), 1.60-1.42 (4H, m).

### Reference Production Example 16

1.16 g of Dess-Martin periodinane was added to a mixture of 700 mg of Intermediate 73 and 20 mL of chloroform, and the mixture was stirred at room temperature for 2 hours and 30 minutes. An aqueous sodium bicarbonate solution and an aqueous sodium thiosulfate solution were added to the resulting mixture, and the mixture was extracted with MTBE. The resulting organic layer was dried over anhydrous sodium sulfate and concentrated under reduced pressure. The resulting residue was subjected to to provide 550 mg of Intermediate 75 represented by the following formula.

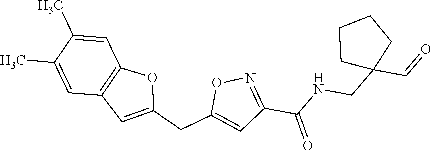 [SMILES: Cc1cc2cc(Cc3cc(C(=O)NCC4(C=O)CCCC4)no3)oc2cc1C]

Intermediate 75: ¹H-NMR (CDCl₃) δ:9.42 (1H, s), 7.26 (1H, s), 7.24-7.15 (2H, m), 6.55 (1H, s), 6.46 (1H, s), 4.27 (2H, s), 3.58 (2H, d), 2.34 (3H, s), 2.32 (3H, s), 1.91-1.70 (6H, m), 1.66-1.58 (2H, m).

### Reference Production Example 17

210 mg of t-butyl [1-(aminomethyl)cyclopentyl]carbamate was added to a mixture of 200 mg of Intermediate 48, 190 mg of 1-ethyl-3-(3-dimethylaminopropyl) carbodiimide hydrochloride, 10 mg of 1-hydroxybenzotriazole, and 5 mL of chloroform, and the mixture was stirred at room temperature for 16 hours. The resulting mixture was concentrated under reduced pressure. The resulting residue was subjected to silica gel chromatography (hexane:ethyl acetate=3:1) to provide 350 mg of Intermediate 76 represented by the following formula.

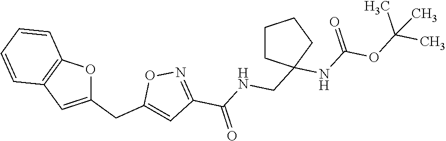 [SMILES: CC(C)(C)OC(=O)NC1(CNC(=O)c2cc(Cc3cc4ccccc4o3)on2)CCCC1]

Intermediate 76: ¹H-NMR (CDCl₃) δ:7.63-7.54 (1H, m), 7.52 (1H, d), 7.44 (1H, d), 7.30-7.19 (2H, m), 6.90 (1H, s), 6.59 (1H, s), 4.68-4.61 (1H, m), 4.32 (2H, s), 3.71 (2H, d), 1.86-1.65 (8H, m), 1.44 (9H, s).

### Reference Production Example 17-1

The compound produced in accordance with Reference production example 17 and physical properties thereof are shown below.

A compound represented by formula (A-4),

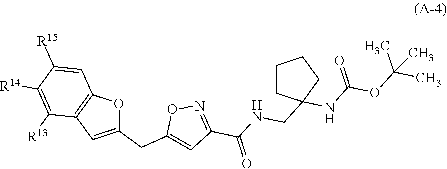

wherein the combination of R¹³, R¹⁴, and R¹⁵ is any one of the combinations shown in [Table A-4].

TABLE A-4

| Intermediate | R¹³ | R¹⁴ | R¹⁵ |
|---|---|---|---|
| 77 | H | H | Me |
| 78 | H | Me | Me |
| 79 | Me | Cl | Me |

Intermediate 77: ¹H-NMR (CDCl₃) δ:7.67-7.48 (1H, m), 7.38 (1H, d), 7.24 (1H, s), 7.04 (1H, d), 6.58 (1H, s), 6.53 (1H, s), 4.73-4.57 (1H, m), 4.29 (2H, s), 3.71 (2H, d), 2.45 (3H, s), 1.88-1.66 (8H, m), 1.44 (9H, s).

Intermediate 78: ¹H-NMR (CDCl₃) δ:7.62-7.50 (1H, m), 7.26 (1H, s), 7.21 (1H, s), 6.57 (1H, s), 6.47 (1H, s), 4.69-4.60 (1H, m), 4.28 (2H, s), 3.71 (2H, d), 2.34 (3H, s), 2.32 (3H, s), 1.87-1.65 (8H, m), 1.44 (9H, s).

Intermediate 79: ¹H-NMR (CDCl₃) δ:7.67-7.64 (1H, m), 7.17 (1H, s), 6.59 (1H, s), 6.54 (1H, s), 4.69-4.58 (1H, m), 4.29 (2H, s), 3.71 (2H, d), 2.49 (3H, s), 2.46 (3H, s), 1.87-1.63 (8H, m), 1.44 (9H, s).

### Reference Production Example 18

A mixture of 3.0 g of potassium cyanide and 10 mL of water was added to a mixture of 3.52 g of cyclopentanone, 4.90 g of N-methoxy-N-methylamine hydrochloride, and 5 mL of water under ice cooling, and the mixture was stirred at room temperature for 2 hours. The resulting mixture was extracted with MTBE. The resulting organic layer was dried over anhydrous sodium sulfate and concentrated under reduced pressure to provide 6.32 g of Intermediate 80 represented by the following formula.

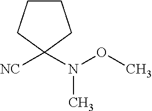

Intermediate 80: ¹H-NMR (CDCl₃) δ:3.55 (3H, s), 2.68 (3H, s), 2.23-2.05 (2H, m), 1.99-1.73 (6H, m).

### Reference Production Example 19

10 mL of a THE solution of 4.82 g of concentrated sulfuric acid was added to a mixture of 3.73 g of aluminum hydride and 50 mL of THF, and the mixture was stirred at −15° C. for 10 minutes. 6.32 g of Intermediate 80 was added to the resulting mixture and the mixture was stirred at room temperature for 3 hour. To the resulting mixture, 3.73 mL of water, 3.73 mL of sodium hydroxide (15% aqueous solution), and 11.2 mL of water were added in this order at −15° C., and the mixture was stirred at room temperature for 1 hour. The resulting mixture was filtered and concentrated under reduced pressure to provide 6.13 g of Intermediate 81 represented by the following formula.

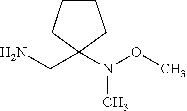

Intermediate 81: ¹H-NMR (CDCl₃) δ:3.48 (3H, s), 2.74-2.63 (2H, m), 2.55 (3H, s), 1.81-1.72 (2H, m), 1.71-1.61 (2H, m), 1.58-1.47 (4H, m), 1.45-1.34 ((2H, m).

### Production Example 1

92 mg of tetrakis(triphenylphosphine) palladium (0) was added to a mixture of 300 mg of Intermediate 3, 420 mg of benzothiophene-2-boronic acid, 440 mg of potassium carbonate, 10 mL of toluene, and 2 mL of water, and the mixture was stirred under heat-reflux for 14 hours. Water was added to the resulting mixture, and the mixture was extracted with MTBE. The resulting organic layer was dried over anhydrous sodium sulfate and concentrated under reduced pressure. The resulting residue was subjected to silica gel chromatography (chloroform:methanol=10:1) to provide 90 mg of Present invention compound 1 represented by the following formula.

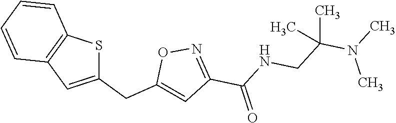 [SMILES: CN(C)C(C)(C)CNC(=O)c1cc(Cc2cc3ccccc3s2)on1]

Compound of the present invention 1: ¹H-NMR (CDCl₃): 7.77 (1H, d), 7.70 (1H, d), 7.48-7.39 (1H, m), 7.36-7.28 (2H, m), 7.16 (1H, s), 6.57 (1H, s), 4.40 (2H, s), 3.33 (2H, d), 2.22 (6H, s), 1.04 (6H, s).

### Production Example 1-1

The compound produced in accordance with Production example 1 and physical properties thereof are shown below. A compound represented by formula (B-1),

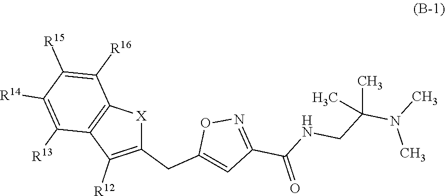

wherein the combination of R¹², R¹³, R¹⁴, R¹⁵, R¹⁶, and X is any one of the combinations shown in [Table B-1].

TABLE B-1

| Compound | | | | | | |
|---|---|---|---|---|---|---|
| of the | | | | | | |
| present | | | | | | |
| invention | R¹² | R¹³ | R¹⁴ | R¹⁵ | R¹⁶ | X |
| 2 | H | H | H | H | H | O |
| 3 | H | H | Cl | H | H | O |
| 4 | H | H | H | Cl | H | O |
| 5 | H | H | C1 | H | H | S |
| 6 | H | H | H | Cl | H | S |
| 7 | H | H | H | Cl | Cl | O |

Compound of the present invention 2: ¹H-NMR (CDCl₃) δ:7.52 (1H, d), 7.49-7.37 (2H, m), 7.29-7.18 (2H, m), 6.60 (1H, s), 6.58 (1H, s), 4.32 (2H, s), 3.33 (2H, d), 2.22 (6H, s), 1.05 (6H, s).

Compound of the present invention 3: ¹H-NMR (CDCl₃) δ: 7.69 (1H, d), 7.67 (1H, d), 7.50-7.41 (1H, m), 7.28 (1H, dd), 7.10 (1H, s), 6.57 (1H, s), 4.40 (2H, s), 3.32 (2H, d), 2.21 (6H, s), 1.04 (6H, s).

Compound of the present invention 4: ¹H-NMR (CDCl₃) δ:7.52-7.37 (3H, m), 7.21 (1H, dd), 6.60 (1H, s), 6.56 (1H, s), 4.31 (2H, s), 3.32 (2H, d), 2.22 (6H, s), 1.05 (6H, s).

Compound of the present invention 5: ¹H-NMR (CDCl₃) δ:7.69 (1H, d), 7.67 (1H, d), 7.53-7.40 (1H, m), 7.27 (1H, dd), 7.10 (1H, s), 6.57 (1H, s), 4.40 (2H, s), 3.33 (2H, d), 2.21 (6H, s), 1.05 (6H, s).

Compound of the present invention 6: ¹H-NMR (CDCl₃) δ:7.75 (1H, d), 7.61 (1H, d), 7.53-7.41 (1H, m), 7.31 (1H, dd), 7.12 (1H, s), 6.57 (1H, s), 4.39 (2H, s), 3.33 (2H, d), 2.21 (6H, s), 1.05 (6H, s).

Compound of the present invention 7: ¹H-NMR (CDCl₃) δ:7.51-7.41 (1H, m), 7.34 (1H, d), 7.32 (1H, d), 6.63 (1H, s), 6.60 (1H, s), 4.36 (2H, s), 3.33 (2H, d), 2.22 (6H, s), 1.05 (6H, s).

### Production Example 2

140 mg of tetrakis(triphenylphosphine) palladium (0) was added to a mixture of 500 mg of Intermediate 2, 550 mg of 5-chlorobenzothiophene-2-boronic acid pinacol ester, 340 mg of potassium carbonate, 10 mL of toluene, and 2 mL of water, and the mixture was stirred under heat-reflux for 3 hours and 30 minutes. Water was added to the resulting mixture, and the mixture was extracted with MTBE. The resulting organic layer was dried over anhydrous sodium sulfate and concentrated under reduced pressure. The resulting residue was subjected to silica gel chromatography (chloroform:methanol=10:1) to provide 140 mg of Compound of the present invention 8 represented by the following formula.

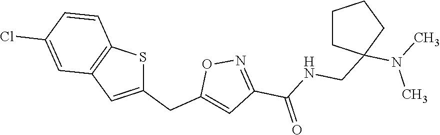 [SMILES: CN(C)C1(CNC(=O)c2cc(Cc3cc4cc(Cl)ccc4s3)on2)CCCC1]

Compound of the present invention 8: ¹H-NMR (CDCl₃) δ:7.71-7.66 (2H, m), 7.50-7.39 (1H, m), 7.29 (1H, dd), 7.10 (1H, s), 6.57 (1H, s), 4.40 (2H, s), 3.41 (2H, d), 2.23 (6H, s), 1.85-1.74 (2H, m), 1.69-1.55 (4H, m), 1.41-1.31 (2H, m).

### Production Example 2-1

The compound produced in accordance with Production example 2 and physical properties thereof are shown below. A compound represented by formula (B-2),

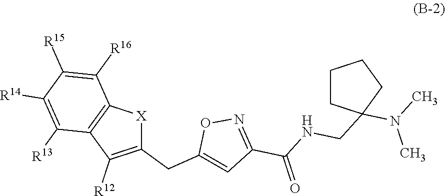

wherein the combination of R¹², R¹³, R¹⁴, R¹⁵, R¹⁶, and X is any one of the combinations shown in [Table B-2].

TABLE B-2

| Compound | | | | | | |
|---|---|---|---|---|---|---|
| of the | | | | | | |
| present | | | | | | |
| invention | R¹² | R¹³ | R¹⁴ | R¹⁵ | R¹⁶ | X |
| 9 | H | H | F | H | H | O |
| 10 | H | H | H | Cl | Cl | O |
| 11 | H | H | OCF³ | H | H | O |
| 12 | H | F | F | F | H | O |
| 13 | H | Cl | Cl | H | H | O |
| 14 | Me | H | H | H | H | O |
| 15 | H | F | H | H | Cl | O |
| 16 | H | H | CF³ | H | H | S |
| 17 | H | H | Me | H | H | O |
| 18 | H | H | OMe | H | H | O |
| 19 | H | H | H | OMe | H | O |
| 20 | H | H | F | H | H | S |
| 21 | H | F | H | H | H | O |
| 22 | H | Me | H | H | H | O |
| 23 | H | H | H | H | F | O |
| 24 | H | H | CN | H | H | O |

Compound of the present invention 9: ¹H-NMR (CDCl₃) δ: 7.51-7.40 (1H, m), 7.35 (1H, dd), 7.17 (1H, dd), 6.98 (1H, td), 6.61 (1H, s), 6.59 (1H, s), 4.31 (2H, s), 3.42 (2H, d), 2.23 (6H, s), 1.86-1.74 (2H, m), 1.69-1.54 (4H, m), 1.41-1.31 (2H, m).

Compound of the present invention 10: ¹H-NMR (CDCl₃) δ:7.54-7.38 (1H, m), 7.34 (1H, d), 7.31 (1H, d), 6.63 (1H, s), 6.60 (1H, s), 4.36 (2H, s), 3.42 (2H, d), 2.23 (6H, s), 1.87-1.76 (2H, m), 1.67-1.56 (4H, m), 1.44-1.31 (2H, m).

Compound of the present invention 11: ¹H-NMR (CDCl₃) δ:7.51-7.40 (2H, m), 7.38 (1H, s), 7.14 (1H, d), 6.61 (1H, s), 6.60 (1H, s), 4.33 (2H, s), 3.42 (2H, d), 2.23 (6H, s), 1.87-1.74 (2H, m), 1.70-1.55 (4H, m), 1.43-1.31 (2H, m).

Compound of the present invention 12: ¹H-NMR (CDCl₃) δ: 7.64 (1H, d), 7.26-7.21 (1H, m), 6.90 (1H, d), 6.43 (1H, s), 4.40 (2H, s), 3.39 (2H, d), 2.21 (6H, s), 1.85-1.73 (2H, m), 1.67-1.55 (4H, m), 1.41-1.29 (2H, m).

Compound of the present invention 13: ¹H-NMR (CDCl₃) δ:7.51-7.40 (1H, m), 7.34 (1H, d), 7.29 (1H, d), 6.69 (1H, s), 6.63 (1H, s), 4.33 (2H, s), 3.42 (2H, d), 2.23 (6H, s), 1.86-1.74 (2H, m), 1.69-1.56 (4H, m), 1.42-1.32 (2H, m).

Compound of the present invention 14: ¹H-NMR (CDCl₃) δ:7.47 (1H, d), 7.45-7.36 (2H, m), 7.30-7.21 (2H, m), 6.51 (1H, s), 4.26 (2H, s), 3.40 (2H, d), 2.24 (3H, s), 2.22 (6H, s), 1.84-1.74 (2H, m), 1.69-1.55 (4H, m), 1.40-1.31 (2H, m).

Compound of the present invention 15: ¹H-NMR (CDCl₃) δ: 7.51-7.38 (1H, m), 7.21 (1H, dd), 6.89 (1H, t), 6.72 (1H, s), 6.64 (1H, s), 4.37 (2H, s), 3.42 (2H, d), 2.23 (6H, s), 1.87-1.74 (2H, m), 1.71-1.58 (4H, m), 1.42-1.31 (2H, m).

Compound of the present invention 16: ¹H-NMR (CDCl₃) δ: 7.98 (1H, s), 7.88 (1H, d), 7.54 (1H, d), 7.50-7.39 (1H, m), 7.25 (1H, s), 6.59 (1H, s), 4.44 (2H, s), 3.41 (2H, d), 2.23 (6H, s), 1.86-1.75 (2H, m), 1.70-1.60 (4H, m), 1.41-1.31 (2H, m).

Compound of the present invention 17: ¹H-NMR (CDCl₃) δ:7.50-7.36 (1H, m), 7.31 (1H, d), 7.29 (1H, s), 7.07 (1H, d), 6.59 (1H, s), 6.50 (1H, s), 4.29 (2H, s), 3.42 (2H, d), 2.42 (3H, s), 2.23 (6H, s), 1.85-1.73 (2H, m), 1.69-1.54 (4H, m), 1.43-1.30 (2H, m).

Compound of the present invention 18: ¹H-NMR (CDCl₃) δ:7.48-7.44 (1H, m), 7.32 (1H, d), 6.98 (1H, d), 6.87 (1H, dd), 6.60 (1H, s), 6.52 (1H, s), 4.29 (2H, s), 3.84 (3H, s), 3.41 (2H, d), 2.23 (6H, s), 1.86-1.74 (2H, m), 1.68-1.54 (4H, m), 1.42-1.27 (2H, m).

Compound of the present invention 19: ¹H-NMR (CDCl₃) δ:7.48-7.40 (1H, m), 7.37 (1H, d), 6.98 (1H, d), 6.85 (1H, dd), 6.59 (1H, s), 6.51 (1H, s), 4.28 (2H, s), 3.84 (3H, s), 3.41 (2H, d), 2.23 (6H, s), 1.85-1.73 (2H, m), 1.67-1.54 (4H, m), 1.42-1.28 (2H, m).

Compound of the present invention 20: ¹H-NMR (CDCl₃) δ:7.70 (1H, dd), 7.51-7.40 (1H, m), 7.36 (1H, dd), 7.12 (1H, s), 7.07 (1H, td), 6.58 (1H, s), 4.40 (2H, s), 3.42 (2H, d), 2.23 (6H, s), 1.87-1.75 (2H, m), 1.71-1.53 (4H, m), 1.43-1.29 (2H, m).

Compound of the present invention 21: ¹H-NMR (CDCl₃) δ: 7.50-7.40 (1H, m), 7.26-7.18 (2H, s), 6.95-6.88 (1H, m), 6.69 (1H, s), 6.62 (1H, s), 4.33 (2H, s), 3.42 (2H, d), 2.23 (6H, s), 1.87-1.74 (2H, m), 1.70-1.52 (4H, m), 1.42-1.31 (2H, m).

Compound of the present invention 22: ¹H-NMR (CDCl₃) δ:7.50-7.39 (1H, m), 7.27 (1H, d), 7.16 (1H, t), 7.01 (1H, d), 6.61 (1H, s), 6.59 (1H, s), 4.32 (2H, s), 3.42 (2H, d), 2.48 (3H, s), 2.23 (6H, s), 1.85-1.75 (2H, m), 1.69-1.56 (4H, m), 1.43-1.32 (2H, m).

Compound of the present invention 23: ¹H-NMR (CDCl₃) δ: 7.52-7.39 (1H, m), 7.28 (1H, d), 7.14 (1H, td), 7.01 (1H, dd), 6.62 (2H, s), 4.35 (2H, s), 3.42 (2H, d), 2.23 (6H, s), 1.86-1.43 (2H, m), 1.71-1.56 (4H, m), 1.42-1.31 (2H, m).

Compound of the present invention 24: ¹H-NMR (CDCl₃) δ:7.87 (1H, s), 7.57 (1H, d), 7.52 (1H, d), 7.50-7.39 (1H, m), 6.67 (1H, s), 6.63 (1H, s), 4.36 (2H, s), 3.42 (2H, d), 2.23 (6H, s), 1.86-1.74 (2H, m), 1.71-1.56 (4H, m), 1.43-1.31 (2H, m).

### Production Example 3

150 mg of (1-amino-2-methylpropane-2-yl) (ethyl)methylamine was added to a mixture of 200 mg of Intermediate 52, 220 mg of 1-ethyl-3-(3-dimethylaminopropyl) carbodiimide hydrochloride, 10 mg of 1-hydroxybenzotriazole, and 2 mL of chloroform, and the mixture was stirred at room temperature for 14 hours and 30 minutes. The resulting mixture was concentrated under reduced pressure. The resulting residue was subjected to silica gel chromatography (chloroform:methanol=10:1) to provide 200 mg of Compound of the present invention 25 represented by the following formula.

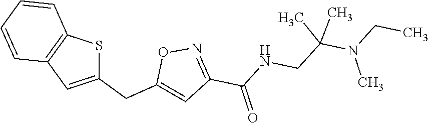 [SMILES: CCN(C)C(C)(C)CNC(=O)c1cc(Cc2cc3ccccc3s2)on1]

Compound of the present invention 25: ¹H-NMR (CDCl₃) 5:7.78 (1H, d), 7.71 (1H, d), 7.60-7.47 (1H, m), 7.37-7.28 (2H, m), 7.16 (1H, s), 6.57 (1H, s), 4.40 (2H, s), 3.32 (2H, d), 2.40 (2H, q), 2.19 (3H, s), 1.07 (3H, t), 1.06 (6H, s).

### Production Example 3-1

The compound produced in accordance with Production example 3 and physical properties thereof are shown below.

A compound represented by formula (B-3),

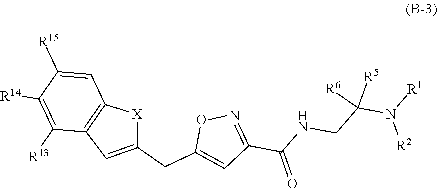

wherein the combination of R¹, R², R⁵, R⁶, R¹³, R¹⁴, R¹⁵, and X is any one of the combinations shown in [Table B-3].

TABLE B-3

| Compound | | | | | | | | |
|---|---|---|---|---|---|---|---|---|
| of the | | | | | | | | |
| present | | | | | | | | |
| invention | R¹ | R² | R⁵ | R⁶ | R¹³ | R¹⁴ | R¹⁵ | X |
| 26 | Et | Me | Me | Me | H | H | H | O |
| 27 | Me | Me | Me | Me | Cl | Cl | H | O |
| 28 | Me | Me | Me | Me | H | F | H | O |
| 29 | Et | Me | Me | Me | H | F | H | O |
| 30 | Me | Me | Me | Me | H | Me | H | O |
| 31 | Et | Me | Me | Me | H | Me | H | O |
| 32 | Me | Me | Et | Et | H | Me | Me | O |
| 33 | OMe | Me | Me | Me | H | Me | Me | O |
| 34 | Me | Me | i-Pr | Me | H | Me | Me | O |
| 35 | —(CH₂)₂O(CH₂)₂— | | Me | Me | H | Me | Me | O |
| 36 | —(CH₂)₄— | | Me | Me | H | Me | Me | O |
| 37 | H | H | Me | Me | H | Me | Me | O |
| 38 | Et | Et | Me | Me | H | Me | Me | O |
| 39 | Bu | Me | Me | Me | H | Me | Me | O |
| 40 | t-Bu | H | Me | Me | H | Me | Me | O |

| 41 | Me | Me | —(CH₂)₄— | H | H | H | O |
|---|---|---|---|---|---|---|---|
| 42 | Me | Me | —(CH₂)₄— | H | H | H | S |
| 43 | Et | Me | —(CH₂)₄— | H | H | H | S |
| 44 | Et | Me | —(CH₂)₄— | H | H | H | O |
| 45 | Me | Me | —(CH₂)₄— | H | H | Cl | S |
| 46 | Me | H | —(CH₂)₄— | H | F | H | O |
| 47 | Me | Me | —(CH₂)₄— | H | H | F | O |
| 48 | Et | Me | —(CH₂)₄— | H | Me | H | O |
| 49 | Me | Me | —(CH₂)₄— | H | H | Me | O |
| 50 | Me | Me | —(CH₂)₄— | Me | H | Me | O |
| 51 | Et | Me | —(CH₂)₄— | H | H | Me | O |
| 52 | Me | H | —(CH₂)₄— | H | Me | H | O |
| 53 | H | H | —(CH₂)₄— | H | Me | H | O |
| 54 | Me | H | —(CH₂)₄— | H | Me | Me | O |
| 55 | Et | H | —(CH₂)₄— | H | Me | Me | O |
| 56 | OMe | Me | —(CH₂)₄— | H | Me | Me | O |
| 57 | Et | Me | —(CH₂)₄— | H | Me | Me | S |
| 58 | Me | H | —(CH₂)₄— | H | H | H | O |
| 59 | —(CH₂)₄— | | —(CH₂)₄— | H | Me | Me | O |
| 60 | Et | H | —(CH₂)₄— | H | H | Me | O |
| 61 | —(CH₂)₄— | | —(CH₂)₄— | H | H | Me | O |
| 62 | i-Pr | H | —(CH₂)₄— | H | Me | Me | O |
| 63 | Et | H | —(CH₂)₄— | Me | Cl | Me | O |
| 64 | i-Pr | H | —(CH₂)₄— | Me | Cl | Me | O |
| 65 | i-Pr | H | —(CH₂)₄— | Me | H | Me | O |
| 66 | i-Pr | H | —(CH₂)₄— | H | Cl | Me | O |
| 67 | Me | Me | —(CH₂)₃— | H | Me | H | O |
| 68 | Me | Me | —(CH₂)₃— | H | Me | Me | O |
| 69 | Et | Me | —(CH₂)₃— | H | Me | Me | O |
| 70 | H | H | —(CH₂)₂— | H | Me | Me | O |
| 71 | Me | Me | —(CH₂)₂OCH₂— | H | Me | Me | O |
| 72 | i-Pr | H | —(CH₂)₄— | H | H | H | O |
| 73 | i-Pr | H | —(CH₂)₄— | H | Me | H | O |
| 74 | i-Pr | H | —(CH₂)₄— | H | Et | H | O |
| 75 | i-Pr | H | —(CH₂)₄— | H | i-Pr | H | O |
| 76 | i-Pr | H | —(CH₂)₄— | H | H | F | O |
| 77 | i-Pr | H | —(CH₂)₄— | H | F | H | O |
| 78 | i-Pr | H | —(CH₂)₄— | H | Cl | H | O |
| 79 | i-Pr | H | —(CH₂)₄— | H | H | OMe | O |
| 80 | i-Pr | H | —(CH₂)₄— | H | Me | OMe | O |
| 81 | i-Pr | H | —(CH₂)₄— | H | H. | Me | O |
| 82 | i-Pr | H | —(CH₂)₄— | H | Ph | H | O |
| 83 | i-Pr | H | —(CH₂)₄— | H | Me | Me | S |
| 84 | i-Pr | H | —(CH₂)₄— | Me | Me | H | S |

Compound of the present invention 26: ¹H-NMR (CDCl₃) δ:7.60-7.47 (2H, m), 7.43 (1H, d), 7.31-7.17 (2H, m), 6.60 (1H, s), 6.59 (1H, s), 4.32 (2H, s), 3.32 (2H, d), 2.40 (2H, q), 2.19 (3H, s), 1.07 (3H, t), 1.06 (6H, s).

Compound of the present invention 27: ¹H-NMR (CDCl₃) δ: 7.52-7.42 (1H, m), 7.34 (1H, d), 7.29 (1H, d), 6.69 (1H, s), 6.63 (1H, s), 4.33 (2H, s), 3.33 (2H, d), 2.22 (6H, s), 1.05 (6H, s).

Compound of the present invention 28: ¹H-NMR (CDCl₃) δ:7.53-7.40 (1H, m), 7.36 (1H, dd), 7.17 (1H, dd), 6.98 (1H, td), 6.61 (1H, s), 6.56 (1H, s), 4.31 (2H, s), 3.33 (2H, d), 2.22 (6H, s), 1.05 (6H, s).

Compound of the present invention 29: ¹H-NMR (CDCl₃) δ:7.62-7.48 (1H, m), 7.35 (1H, dd), 7.17 (1H, dd), 6.98 (1H, td), 6.60 (1H, s), 6.56 (1H, s), 4.31 (2H, s), 3.32 (2H, d), 2.40 (2H, q), 2.19 (3H, s), 1.07 (3H, t), 1.06 (6H, s).

Compound of the present invention 30: ¹H-NMR (CDCl₃) δ:7.51-7.40 (1H, m), 7.30 (1H, d), 7.29 (1H, s), 7.07 (1H, d), 6.59 (1H, s), 6.50 (1H, s), 4.30 (2H, s), 3.33 (2H, d), 2.42 (3H, s), 2.22 (6H, s), 1.05 (6H, s).

Compound of the present invention 31: ¹H-NMR (CDCl₃) δ: 7.62-7.47 (1H, m), 7.30 (1H, d), 7.29 (1H, s), 7.07 (1H, d), 6.59 (1H, s), 6.51 (1H, s), 4.29 (2H, s), 3.32 (2H, d), 2.42 (3H, s), 2.40 (2H, q), 2.19 (3H, s), 1.07 (3H, t), 1.06 (6H, s).

Compound of the present invention 32: ¹H-NMR (CDCl₃) δ: 7.54-7.40 (1H, m), 7.26 (1H, d), 7.21 (1H, s), 6.57 (1H, s), 6.47 (1H, s), 4.27 (2H, s), 3.34 (2H, d), 2.34 (3H, s), 2.33 (6H, s), 2.31 (3H, s), 1.68-1.56 (2H, m), 1.51-1.39 (2H, m), 0.89 (6H, t).

Compound of the present invention 33: ¹H-NMR (CDCl₃) δ:7.50-7.37 (1H, m), 7.26 (1H, d), 7.22 (1H, s), 6.58 (1H, s), 6.47 (1H, s), 4.28 (2H, s), 3.51 (3H, s), 3.45 (2H, d), 2.54 (3H, s), 2.34 (3H, s), 2.32 (3H, s), 1.08 (6H, s).

Compound of the present invention 34: ¹H-NMR (CDCl₃) δ:7.56-7.49 (1H, m), 7.26 (1H, d), 7.21 (1H, s), 6.57 (1H, s), 6.47 (1H, s), 4.27 (2H, s), 3.34 (2H, d), 2.34 (9H, s); 2.31 (3H, s), 2.05-1.94 (1H, m), 1.00-0.87 (9H, m).

Compound of the present invention 35: ¹H-NMR (CDCl₃) δ: 7.54-7.41 (1H, m), 7.26 (1H, d), 7.21 (1H, s), 6.58 (1H, s), 6.48 (1H, s), 4.28 (2H, s), 3.73 (4H, t), 3.34 (2H, d), 2.54 (4H, t), 2.34 (3H, s), 2.32 (3H, s), 1.08 (6H, s).

Compound of the present invention 36: ¹H-NMR (CDCl₃) δ:7.57-7.35 (1H, m), 7.26 (1H, s), 7.21 (1H, s), 6.58 (1H, s), 6.47 (1H, s), 4.27 (2H, s), 3.34 (2H, d), 2.72-2.49 (4H, m), 2.34 (3H, s), 2.31 (3H, s), 1.82-1.69 (4H, m), 1.08 (6H, s).

Compound of the present invention 37: ¹H-NMR (CDCl₃) δ:7.28-7.23 (1H, m), 7.26 (1H, d), 7.21 (1H, s), 6.58 (1H, s), 6.47 (1H, s), 4.28 (2H, s), 3.31 (2H, d), 2.34 (3H, s), 2.32 (3H, s), 1.48-1.28 (2H, m), 1.16 (6H, s).

Compound of the present invention 38: ¹H-NMR (CDCl₃) δ:7.64-7.57 (1H, m), 7.26 (1H, d), 7.21 (1H, s), 6.57 (1H, s), 6.47 (1H, s), 4.27 (2H, s), 3.27 (2H, d), 2.56 (4H, q), 2.34 (3H, s), 2.32 (3H, s), 1.10 (6H, s), 1.06 (6H, t).

Compound of the present invention 39: ¹H-NMR (CDCl₃) δ: 7.55-7.49 (1H, m), 7.26 (1H, d), 7.21 (1H, s), 6.57 (1H, s), 6.47 (1H, s), 4.27 (2H, s), 3.32 (2H, d), 2.37-2.30 (2H, m), 2.34 (3H, s), 2.32 (3H, s), 2.18 (3H, s), 1.48-1.39 (2H, m), 1.37-1.26 (2H, m), 1.05 (6H, s), 0.90 (3H, c).

Compound of the present invention 40: ¹H-NMR (CDCl₃) δ:7.77-7.68 (1H, m), 7.26 (1H, d), 7.21 (1H, s), 6.58 (1H, s), 6.47 (1H, s), 4.27 (2H, s), 3.23 (2H, d), 2.34 (3H, S), 2.31 (3H, s), 1.21 (6H, s), 1.23 (9H, s).

Compound of the present invention 41: ¹H-NMR (CDCl₃) δ:7.52 (1H, d), 7.50-7.36 (2H, m), 7.31-7.17 (2H, m), 6.60 (1H, s), 6.58 (1H, s), 4.32 (2H, s), 3.42 (2H, d), 2.23 (6H, s), 1.87-1.73 (2H, m), 1.72-1.53 (4H, m), 1.44-1.30 (2H, m).

Compound of the present invention 42: ¹H-NMR (CDCl₃) δ: 7.78 (1H, d), 7.70 (1H, d), 7.50-7.39 (1H, m), 7.37-7.28 (2H, m), 7.16 (1H, s), 6.57 (1H, s), 4.40 (2H, s), 3.41 (2H, d), 2.23 (6H, s), 1.87-1.72 (2H, m), 1.70-1.55 (4H, m), 1.42-1.31 (2H, m).

Compound of the present invention 43: ¹H-NMR (CDCl₃) δ:7.78 (1H, d), 7.70 (1H, d), 7.50-7.39 (1H, m), 7.37-7.28 (2H, m), 7.16 (1H, s), 6.57 (1H, s), 4.40 (2H, s), 3.41 (2H, d), 2.43 (2H, q), 2.23 (3H, s), 1.85-1.72 (2H, m), 1.70-1.55 (4H, m), 1.49-1.35 (2H, m), 1.06 (3H, t).

Compound of the present invention 44: ¹H-NMR (CDCl₃) δ:7.52 (1H, d), 7.50-7.38 (2H, m), 7.29-7.19 (2H, m), 6.60 (1H, s), 6.59 (1H, s), 4.32 (2H, s), 3.42 (2H, d), 2.44 (2H, q), 2.23 (3H, s), 1.86-1.72 (2H, m), 1.70-1.54 (4H, m), 1.49-1.34 (2H, m), 1.06 (3H, t).

Compound of the present invention 45: ¹H-NMR (CDCl₃) δ: 7.76 (1H, d), 7.61 (1H, d), 7.49-7.40 (1H, m), 7.31 (1H, dd), 7.12 (1H, s), 6.57 (1H, s), 4.31 (2H, s), 3.41 (2H, d), 2.23 (6H, s), 1.86-1.73 (2H, m), 1.70-1.54 (4H, m), 1.42-1.30 (2H, m).

Compound of the present invention 46: ¹H-NMR (CDCl₃) δ:7.56-7.41 (1H, m), 7.35 (1H, dd), 7.17 (1H, dd), 6.98 (1H, td), 6.60 (1H, s), 6.55 (1H, s), 4.30 (2H, s), 3.42 (2H, d), 2.34 (3H, s), 1.76-1.45 (8H, m).

Compound of the present invention 47: ¹H-NMR (CDCl₃) δ: 7.50-7.37 (2H, m), 7.16 (1H, dd), 6.99 (1H, td), 6.60 (1H, s), 6.56 (1H, s), 4.30 (2H, s), 3.42 (2H, d), 2.23 (6H, s), 1.86-1.73 (2H, m), 1.70-1.53 (4H, m), 1.42-1.29 (2H, m).

Compound of the present invention 48: ¹H-NMR (CDCl₃) δ:7.51-7.41 (1H, m), 7.31 (1H, d), 7.29 (1H, s), 7.07 (1H, d), 6.59 (1H, s), 6.51 (1H, s), 4.29 (2H, s), 3.42 (2H, d), 2.43 (2H, q), 2.42 (3H, s), 2.23 (3H, s), 1.83-1.71 (2H, m), 1.69-1.55 (4H, m), 1.48-1.37 (2H, m), 1.06 (3H, t).

Compound of the present invention 49: ¹H-NMR (CDCl₃) δ: 7.48-7.40 (1H, m), 7.38 (1H, d), 7.24 (1H, s), 7.04 (1H, d), 6.58 (1H, s), 6.52 (1H, s), 4.29 (2H, s), 3.42 (2H, d) 2.46 (3H, s), 2.23 (6H, s), 1.85-1.75 (2H, m), 1.69-1.54 (4H, m), 1.42-1.31 (2H, m).

Compound of the present invention 50: ¹H-NMR (CDCl₃) δ:7.49-7.39 (1H, m), 7.07 (1H, s), 6.85 (1H, s), 6.59 (1H, s), 6.53 (1H, s), 4.29 (2H, s), 3.42 (2H, d), 2.43 (3H, s), 2.41 (3H, s), 2.23 (6H, s), 1.85-1.73 (2H, m), 1.71-1.55 (4H, m), 1.42-1.31 (2H, m).

Compound of the present invention 51: ¹H-NMR (CDCl₃) δ:7.50-7.41 (1H, m), 7.38 (1H, d), 7.24 (1H, s), 7.04 (1H, d), 6.58 (1H, s), 6.53 (1H, s), 4.28 (2H, s), 3.41 (2H, d), 2.45 (3H, s), 2.43 (2H, q), 2.23 (3H, s), 1.83-1.71 (2H, m), 1.70-1.54 (4H, m), 1.49-1.36 (2H, m), 1.06 (3H, t).

Compound of the present invention 52: ¹H-NMR (CDCl₃) δ:7.81-7.48 (1H, m), 7.30 (1H, d), 7.28 (1H, s), 7.06 (1H, d), 6.58 (1H, s), 6.48 (1H, s), 4.26 (2H, s), 3.45 (2H, d), 2.42 (3H, s), 2.37 (3H, s), 1.76-1.47 (8H, m).

Compound of the present invention 53: ¹H-NMR (CDCl₃) δ:7.40-7.28 (3H, m), 7.07 (1H, d), 6.60 (1H, s), 6.50 (1H, s), 4.29 (2H, s), 3.40 (2H, d), 2.42 (3H, s), 1.85-1.31 (10H, m).

Compound of the present invention 54: ¹H-NMR (CDCl₃) δ:7.75-7.51 (1H, m), 7.25 (1H, d), 7.20 (1H, s), 6.56 (1H, s), 6.44 (1H, s), 4.23 (2H, s), 3.45 (2H, d), 2.38 (3H, s), 2.34 (3H, s), 2.31 (3H, s), 1.77-1.50 (8H, m).

Compound of the present invention 55: ¹H-NMR (CDCl₃) δ: 7.51-7.37 (1H, m), 7.26 (1H, s), 7.21 (1H, s), 6.57 (1H, s), 6.47 (1H, s), 4.27 (2H, s), 3.37 (2H, d), 2.55 (2H, q), 2.34 (3H, s), 2.31 (3H, s), 1.73-1.50 (8H, m), 1.09 (3H, t).

Compound of the present invention 56: ¹H-NMR (CDCl₃) δ:7.75-7.64 (1H, m), 7.26 (1H, s), 7.21 (1H, s), 6.58 (1H, s), 6.47 (1H, s), 4.28 (2H, s), 3.53 (2H, d), 3.52 (3H, s), 2.60 (3H, s), 2.34 (3H, s), 2.32 (3H, s), 1.82-1.59 (6H, m), 1.56-1.43 (2H, m).

Compound of the present invention 57: ¹H-NMR (CDCl₃) δ:7.53 (1H, s), 7.49-7.40 (2H, m), 7.04 (1H, s), 6.55 (1H, s), 4.36 (2H, s), 3.41 (2H, d), 2.43 (2H, q), 2.35 (3H, s), 2.34 (3H, s), 2.23 (3H, s), 1.82-1.72 (2H, m), 1.70-1.59 (4H, m), 1.49-1.37 (2H, m), 1.06 (3H, t).

Compound of the present invention 58: ¹H-NMR (CDCl₃) δ: 7.52 (1H, d), 7.49-7.34 (2H, m), 7.27 (1H, t), 7.21 (1H, t), 6.60 (1H, s), 6.58 (1H, s), 4.31 (2H, s), 3.40 (2H, d), 2.32 (3H, s), 1.78-1.46 (8H, m).

Compound of the present invention 59: ¹H-NMR (CDCl₃) δ:7.59-7.46 (1H, m), 7.26 (1H, s), 7.21 (1H, s), 6.57 (1H, s), 6.47 (1H, s), 4.28 (2H, s), 3.38 (2H, d), 2.60 (4H, t), 2.34 (3H, s), 2.32 (3H, s), 1.85-1.70 (6H, m), 1.69-1.59 (4H, m), 1.47-1.34 (2H, m).

Compound of the present invention 60: ¹H-NMR (CDCl₃) δ:7.53-7.40 (1H, m), 7.38 (1H, d), 7.24 (1H, s), 7.04 (1H, d), 6.58 (1H, s), 6.52 (1H, s), 4.29 (2H, s), 3.36 (2H, d), 2.54 (2H, q), 2.46 (3H, s), 1.76-1.46 (8H, m), 1.09 (3H, t).

Compound of the present invention 61: ¹H-NMR (CDCl₃) δ:7.60-7.50 (1H, m), 7.38 (1H, d), 7.24 (1H, s), 7.04 (1H, d), 6.59 (1H, s), 6.52 (1H, s), 4.29 (2H, s), 3.38 (2H, d), 2.66-2.53 (4H, m), 2.46 (3H, s), 1.84-1.70 (6H, m), 1.68-1.54 (4H, m), 1.47-1.34 (2H, m).

Compound of the present invention 62: ¹H-NMR (CDCl₃) δ:7.56-7.50 (1H, m), 7.26 (1H, s), 7.21 (1H, s), 6.57 (1H, s), 6.47 (1H, s), 4.27 (2H, s), 3.34 (2H, d), 2.94-2.83 (1H, m), 2.34 (3H, s), 2.32 (3H, s), 1.78-1.43 (9H, m), 1.07 (6H, d).

Compound of the present invention 63: ¹H-NMR (CDCl₃) δ:7.60-7.36 (1H, m), 7.17 (1H, s), 6.59 (1H, s), 6.53 (1H, s), 4.28 (2H, s), 3.38 (2H, d), 2.65-2.51 (2H, m), 2.49 (3H, s), 2.46 (3H, s), 1.71-1.52 (8H, m), 1.10 (3H, t).

Compound of the present invention 64: ¹H-NMR (CDCl₃) δ:7.59-7.51 (1H, m), 7.17 (1H, s), 6.60 (1H, s), 6.53 (1H, s), 4.29 (2H, s), 3.34 (2H, d), 2.95-2.82 (1H, m), 2.49 (3H, s), 2.46 (3H, s), 1.74-1.50 (8H, m), 1.07 (6H, d).

Compound of the present invention 65: ¹H-NMR (CDCl₃) δ: 7.58-7.50 (1H, m), 7.07 (1H, s), 6.85 (1H, s), 6.59 (1H, s), 6.53 (1H, s), 4.29 (2H, s), 3.34 (2H, d), 2.94-2.83 (1H, m), 2.43 (3H, s), 2.41 (3H, s), 1.75-1.61 (4H, m), 1.60-1.50 (4H, m), 1.07 (6H, d).

Compound of the present invention 66: ¹H-NMR (CDCl₃) δ:7.59-7.51 (1H, m), 7.48 (1H, s), 7.30 (1H, s), 6.59 (1H, s), 6.49 (1H, s), 4.28 (2H, s), 3.34 (2H, d), 2.95-2.83 (1H, m), 2.45 (3H, s), 1.76-1.61 (4H, m), 1.60-1.50 (4H, m), 1.07 (6H, d).

Compound of the present invention 67: ¹H-NMR (CDCl₃) δ: 7.31 (1H, d), 7.29 (1H, s), 7.28-7.18 (1H, m), 7.07 (1H, d), 6.60 (1H, s), 6.51 (1H, s), 4.29 (2H, s), 3.41 (2H, d), 2.42 (3H, s), 2.31-2.16 (2H, m), 2.24 (6H, s), 1.83-1.57 (4H, m).

Compound of the present invention 68: ¹H-NMR (CDCl₃): 7.32-7.22 (2H, m), 7.21 (1H, s), 6.58 (1H, s), 6.47 (1H, s), 4.27 (2H, s), 3.61 (2H, d), 2.34 (3H, s), 2.32 (3H, s), 2.28-2.16 (2H, m), 2.24 (6H, s), 1.84-1.62 (4H, m).

Compound of the present invention 69: ¹H-NMR (CDCl₃) δ: 7.26 (1H, s), 7.21 (1H, s), 7.20-7.13 (1H, m), 6.58 (1H, s), 6.47 (1H, s), 4.27 (2H, s), 3.62 (2H, d), 2.46 (2H, q), 2.34 (3H, s), 2.32 (3H, s), 2.23 (3H, s), 2.20-2.10 (2H, m), 1.84-1.70 (4H, m), 1.05 (3H, t).

Compound of the present invention 70: ¹H-NMR (CDCl₃) δ: 7.26 (1H, s), 7.21 (1H, s), 7.15-7.05 (1H, m), 6.60 (1H, s), 6.48 (1H, s), 4.29 (2H, s), 3.42 (2H, d), 2.35 (3H, s), 2.32 (3H, s), 1.70-1.52 (2H, m), 0.69-0.63 (2H, m), 0.62-0.57 (2H, m).

Compound of the present invention 71: ¹H-NMR (CDCl₃) δ:7.43-7.34 (1H, m), 7.26 (1H, s), 7.21 (1H, s), 6.58 (1H, s), 6.47 (1H, s), 4.28 (2H, s), 3.97 (1H, td), 3.91 (1H, d), 3.84 (1H, q), 3.66 (1H, dd), 3.55 (1H, d), 3.52 (1H, d), 2.34 (3H, s), 2.32 (3H, s), 2.29 (6H, s), 2.18-2.08 (1H, m), 1.67-1.58 (1H, m).

Compound of the present invention 72: ¹H-NMR (CDCl₃) δ: 7.60-7.50 (2H, m), 7.43 (1H, d), 7.27 (1H, t), 7.21 (1H, t), 6.60 (1H, s), 6.59 (1H, s), 4.32 (2H, s), 3.34 (2H, d), 2.94-2.84 (1H, m), 1.74-1.48 (8H, m), 1.07 (6H, d).

Compound of the present invention 73: ¹H-NMR (CDCl₃) δ:7.61-7.46 (1H, m), 7.30 (1H, d), 7.29 (1H, s), 7.07 (1H, d), 6.59 (1H, s), 6.50 (1H, s), 4.29 (2H, s), 3.34 (2H, d), 2.95-2.83 (1H, m), 2.42 (3H, s), 1.76-1.49 (8H, m), 1.06 (6H, d).

Compound of the present invention 74: ¹H-NMR (CDCl₃) δ:7.59-7.46 (1H, m), 7.33 (1H, d), 7.32 (1H, s), 7.10 (1H, d), 6.59 (1H, s), 6.52 (1H, s), 4.29 (2H, s), 3.34 (2H, d), 2.94-2.84 (1H, m), 2.72 (2H, q), 1.75-1.50 (8H, m), 1.26 (3H, t), 1.06 (6H, d).

Compound of the present invention 75: ¹H-NMR (CDCl₃) δ: 7.58-7.44 (1H, m), 7.35 (1H, s), 7.34 (1H, d), 7.14 (1H, d), 6.58 (1H, s), 6.53 (1H, s), 4.29 (2H, s), 3.34 (2H, d), 3.05-2.94 (1H, m), 2.93-2.84 (1H, m), 1.74-1.49 (8H, m), 1.28 (6H, d), 1.06 (6H, d).

Compound of the present invention 76: ¹H-NMR (CDCl₃) δ: 7.63-7.48 (1H, m), 7.43 (1H, dd), 7.16 (1H, d), 6.99 (1H, t), 6.60 (1H, s), 6.56 (1H, s), 4.30 (2H, s), 3.34 (2H, d), 2.94-2.84 (1H, m), 1.74-1.50 (8H, m), 1.07 (6H, d).

Compound of the present invention 77: ¹H-NMR (CDCl₃) δ: 7.59-7.51 (1H, m), 7.35 (1H, dd), 7.17 (1H, dd), 6.98 (1H, td), 6.60 (1H, s), 6.56 (1H, s), 4.30 (2H, s), 3.34 (2H, d), 2.95-2.84 (1H, m), 1.75-1.51 (8H, m), 1.07 (6H, d).

Compound of the present invention 78: ¹H-NMR (CDCl₃) δ:7.59-7.51 (1H, m), 7.49 (1H, dd), 7.35 (1H, d), 7.23 (1H, dd), 6.61 (1H, s), 6.54 (1H, s), 4.31 (2H, s), 3.34 (2H, d), 2.94-2.84 (1H, m), 1.74-1.50 (8H, m), 1.07 (6H, d).

Compound of the present invention 79: ¹H-NMR (CDCl₃) δ: 7.57-7.50 (1H, m), 7.37 (1H, d), 6.98 (1H, d), 6.85 (1H, dd), 6.59 (1H, s), 6.51 (1H, s), 4.28 (2H, s), 3.84 (3H, s), 3.34 (2H, d), 2.94-2.84 (1H, m), 1.74-1.48 (8H, m), 1.07 (6H, d).

Compound of the present invention 80: ¹H-NMR (CDCl₃) δ: 7.57-7.47 (1H, m), 7.22 (1H, s), 6.92 (1H, s), 6.57 (1H, s), 6.45 (1H, s), 4.26 (2H, s), 3.85 (3H, s), 3.34 (2H, d), 2.94-2.84 (1H, m), 2.26 (3H, s), 1.75-1.52 (8H, m), 1.06 (6H, d).

Compound of the present invention 81: ¹H-NMR (CDCl₃) δ:7.57-7.50 (1H, m), 7.38 (1H, d), 7.24 (1H, s), 7.04 (1H, d), 6.58 (1H, s), 6.52 (1H, s), 4.29 (2H, s), 3.34 (2H, d), 2.94-2.84 (1H, m), 2.45 (3H, s), 1.74-1.51 (8H, m), 1.07 (6H, d).

Compound of the present invention 82: ¹H-NMR (CDCl₃) δ: 7.70 (1H, s), 7.67-7.52 (3H, m), 7.50-7.40 (4H, m), 7.34 (1H, t), 6.63 (1H, s), 6.62 (1H, s), 4.33 (2H, s), 3.35 (2H, d), 2.94-2.84 (1H, m), 1.74-1.51 (8H, m), 1.07 (6H, d).

Compound of the present invention 83: ¹H-NMR (CDCl₃) δ:7.57-7.48 (2H, m), 7.46 (1H, s), 7.04 (1H, s), 6.54 (1H, s), 4.36 (2H, s), 3.34 (2H, d), 2.94-2.84 (1H, m), 2.35 (3H, s), 2.34 (3H, s), 1.73-1.52 (8H, m), 1.06 (6H, d).

Compound of the present invention 84: ¹H-NMR (CDCl₃) δ: 7.56-7.48 (2H, m), 7.21 (1H, s), 7.12 (1H, d), 6.56 (1H, s), 4.39 (2H, s), 3.34 (2H, d), 2.94-2.84 (1H, m), 2.47 (3H, s), 2.38 (3H, s), 1.76-1.49 (8H, m), 1.06 (6H, d).

### Production Example 3-2

The compound produced in accordance with Production example 3 and physical properties thereof are shown below.

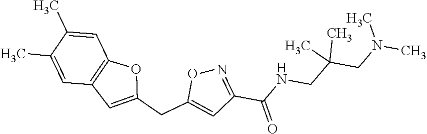

Compound of the present invention 85: ¹H-NMR (CDCl₃) δ: 9.34-9.23 (1H, m), 7.26 (1H, d), 7.21 (1H, s), 6.57 (1H, s), 6.47 (1H, s), 4.26 (2H, s), 3.33 (2H, d), 2.34 (3H, s), 2.33 (6H, s), 2.31 (3H, s), 2.30 (2H, s), 0.96 (6H, s).

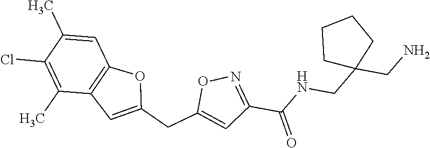 [SMILES: Cc1cc2oc(Cc3cc(C(=O)NCC4(CN)CCCC4)no3)cc2c(C)c1Cl]

Compound of the present invention 165: ¹H-NMR (CDCl₃) δ: 8.52 (1H, s), 7.16 (1H, s), 6.60 (1H, s), 6.53 (1H, s), 4.28 (2H, s), 3.44 (2H, d), 2.76 (2H, s), 2.48 (3H, s), 2.46 (3H, s), 1.87-1.43 (10H, m).

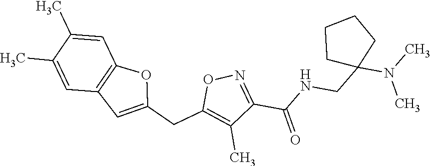

Compound of the present invention 86: ¹H-NMR (CDCl₃) δ:7.46-7.37 (1H, m), 7.23 (1H, s), 7.20 (1H, s), 6.37 (1H, s), 4.21 (2H, s), 3.41 (2H, d), 2.32 (3H, s), 2.31 (3H, s), 2.24 (6H, s), 2.21 (3H, s), 1.87-1.75 (2H, m), 1.72-1.52 (4H, m), 1.47-1.32 (2H, m).

### Production Example 3-3

The compound produced in accordance with Production example 3 and physical properties thereof are shown below.

A compound represented by formula (B-4),

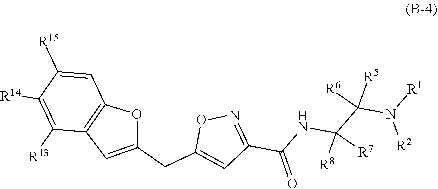

wherein the combination of R¹, R², R⁵, R⁶, R⁷, R⁸, R¹³, R¹⁴, and R¹⁵ is any one of the combinations shown in [Table B-4].

TABLE B-4

| Compound | | | | | | | | | |
|---|---|---|---|---|---|---|---|---|---|
| of the | | | | | | | | | |
| present | | | | | | | | | |
| invention | R¹ | R² | R⁵ | R⁶ | R⁷ | R⁸ | R¹³ | R¹⁴ | R¹⁵ |
| 87 | Me | Me | Et | Me | Me | H | H | Me | Me |

| | | | | | | | | |
|---|---|---|---|---|---|---|---|---|
| 88 | Me | Me | —(CH₂)₄— | Me | H | H | Me | Me |
| 89 | Me | Me | —(CH₂)₄— | Bu | H | H | Me | Me |
| 90 | Me | Me | —(CH₂)₄— | Me | H | Me | Cl | Me |
| 91 | Et | Et | —(CH₂)₄— | Me | H | H | Me | Me |
| 92 | Et | Et | —(CH₂)₄— | Me | H | Me | Cl | Me |

Compound of the present invention 87: ¹H-NMR (CDCl₃) δ:7.72-7.65 (1H, m), 7.26 (1H, d), 7.21 (1H, s), 6.56 (1H, s), 6.47 (1H, s), 4.27 (2H, s), 4.05-3.95 (1H, m), 2.34 (3H, s), 2.34 (3H, s), 2.32 (3H, s), 2.24 (3H, s), 1.66-1.52 (2H, m), 1.31 (3H, d), 1.06-0.88 (6H, m).

Compound of the present invention 88: ¹H-NMR (CDCl₃) δ:7.26 (1H, s), 7.21 (1H, s), 7.05 (1H, d), 6.57 (1H, s), 6.47 (1H, s), 4.45-4.33 (1H, m), 4.27 (2H, s), 2.34 (3H, s), 2.32 (3H, s), 2.22 (6H, s), 1.83-1.34 (8H, m), 1.25 (3H, d).

Compound of the present invention 89: ¹H-NMR (CDCl₃) δ:7.26 (1H, s), 7.22 (1H, s), 6.62 (1H, d), 6.59 (1H, s), 6.48 (1H, s), 4.41 (1H, td), 4.28 (2H, s), 2.35 (3H, s), 2.32 (3H, s), 2.24 (6H, s), 1.96-1.20 (14H, m), 0.86 (3H, t).

Compound of the present invention 90: ¹H-NMR (CDCl₃) δ: 7.17 (1H, s), 7.11-7.01 (1H, m), 6.59 (1H, s), 6.54 (1H, s), 4.44-4.33 (1H, m), 4.29 (2H, s), 2.49 (3H, s), 2.46 (3H, s), 2.22 (6H, s), 1.84-1.49 (8H, m), 1.26 (3H, d).

Compound of the present invention 91: ¹H-NMR (CDCl₃) δ:7.26 (1H, s), 7.21 (1H, s), 7.20-7.12 (1H, m), 6.57 (1H, s), 6.47 (1H, s), 4.27 (2H, s), 4.26-4.20 (1H, m), 2.69-2.55 (4H, m), 2.34 (3H, s), 2.32 (3H, s), 1.79-1.47 (8H, m), 1.24 (3H, d), 1.03 (6H, t).

Compound of the present invention 92: ¹H-NMR (CDCl₃) δ:7.22-7.13 (2H, m), 6.59 (1H, s), 6.54 (1H, s), 4.29 (2H, s), 4.28-4.19 (1H, m), 2.69-2.56 (4H, m), 2.49 (3H, s), 2.46 (3H, s), 1.79-1.47 (8H, m), 1.25 (3H, d), 1.03 (6H, t).

### Production Example 4

To 60 mg of Intermediate 46 was added 70 mg of (1-amino-2-methylpropan-2-yl) (ethyl)methylamine and the mixture was stirred at 140° C. for 2 hours 30 minutes. The resulting mixture was concentrated under reduced pressure. The resulting residue was subjected to silica gel chromatography (chloroform:methanol=10:1) to provide 30 mg of Compound of the present invention 93 represented by the following formula,

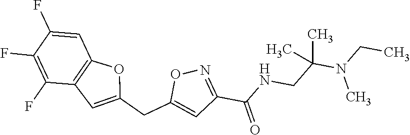 [SMILES: CCN(C)C(C)(C)CNC(=O)c1cc(Cc2cc3c(F)c(F)c(F)cc3o2)on1]

Compound of the present invention 93: ¹H-NMR (CDCl₃) δ:7.64 (1H, d), 7.58-7.46 (1H, m), 6.90 (1H, d), 6.44 (1H, s), 4.40 (2H, s), 3.30 (2H, d), 2.38 (2H, q), 2.17 (3H, s); 1.05 (3H, t), 1.04 (6H, s).

### Production Example 4-1

The compound produced in accordance with Production example 4 and physical properties thereof are shown below.

A compound represented by formula (B-3),

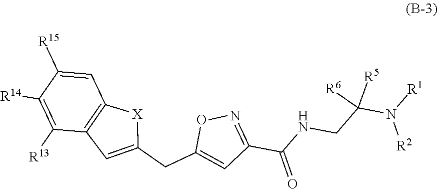

wherein the combination of R¹, R², R⁵, R⁶, R¹³, R¹⁴, R¹⁵, and X is any one of the combinations shown in [Table B-5].

TABLE B-5

| Compound | | | | | | | | |
|---|---|---|---|---|---|---|---|---|
| of the | | | | | | | | |
| present | | | | | | | | |
| invention | R¹ | R² | R⁵ | R⁶ | R¹³ | R¹⁴ | R¹⁵ | X |
| 94 | Me | Me | Me | Me | H | Cl | O | O |
| 95 | Me | Me | Me | Me | H | Me | O | O |
| 96 | Et | Me | Me | Me | H | Me | O | O |
| 97 | Et | Me | Me | Me | Me | C1 | O | O |
| 98 | Et | Me | Me | Me | Me | Me | O | O |
| 99 | Et | Me | —(CH₂)₄— | | H | Cl | Me | O |
| 100 | Me | Me | —(CH₂)₄— | | H | Cl | H | O |
| 101 | Et | Me | —(CH₂)₄— | | H | H | OMe | O |
| 102 | Me | Me | —(CH₂)₄— | | H | Me | Me | O |
| 103 | Et | Me | —(CH₂)₄— | | H | Me | Me | O |
| 104 | Me | Me | —(CH₂)₄— | | H | Et | H | O |
| 105 | Me | Me | —(CH₂)₄— | | H | i-Pr | H | O |
| 106 | Me | Me | —(CH₂)₄— | | H | H | H | NMe |
| 107 | Me | Me | —(CH₂)₄— | | H | Ph | H | O |
| 108 | Me | Me | —(CH₂)₄— | | H | Me | OMe | O |
| 109 | Me | Me | —(CH₂)₄— | | H | Me | Cl | O |
| 110 | Me | Me | —(CH₂)₄— | | Me | Me | Me | O |
| 111 | Me | Me | —(CH₂)₄— | | H | Cl | Cl | O |
| 112 | Me | Me | —(CH₂)₄— | | H | —OCH₂O— | | O |
| 113 | Me | Me | —(CH₂)₄— | | Me | Cl | Me | O |
| 114 | Et | Me | —(CH₂)₄— | | Me | Me | Me | O |
| 115 | Me | Me | —(CH₂)₄— | | H | Cl | Me | O |
| 116 | Et | Me | —(CH₂)₄— | | Me | Cl | Me | O |
| 117 | Me | Me | —(CH₂)₄— | | H | Me | Et | O |
| 118 | OMe | Me | —(CH₂)₄— | | H | Me | H | O |
| 119 | Et | H | —(CH₂)₄— | | H | Me | H | O |
| 120 | Me | Me | —(CH₂)₄— | | Me | Me | H | S |
| 121 | Me | Me | —(CH₂)₄— | | H | Me | Me | S |
| 122 | i-Pr | H | —(CH₂)₄— | | Me | Me | Me | O |
| 170 | Bn | H | —(CH₂)₄— | | H | Me | Me | O |
| 173 | Me | H | Me | Me | H | Me | O | O |

| | | | | | | | |
|---|---|---|---|---|---|---|---|
| 174 | Me | H | —(CH₂)₄— | H | Cl | Me | O |

Compound of the present invention 94: ¹H-NMR (CDCl₃) δ:7.59 (1H, s), 7.55 (1H, s), 7.52-7.41 (1H, m), 6.61 (1H, s), 6.53 (1H, s), 4.31 (2H, s), 3.33 (2H, d), 2.22 (6H, s), 1.05 (6H, s).

Compound of the present invention 95: ¹H-NMR (CDCl₃) δ:7.51-7.40 (1H, m), 7.26 (1H, d), 7.21 (1H, s), 6.57 (1H, s), 6.47 (1H, s), 4.27 (2H, s), 3.33 (2H, d), 2.34 (3H, s), 2.32 (3H, s), 2.22 (6H, s), 1.05 (6H, s).

Compound of the present invention 96: ¹H-NMR (CDCl₃) δ:7.58-7.48 (1H, m), 7.26 (1H, d), 7.21 (1H, s), 6.57 (1H, s), 6.47 (1H, s), 4.27 (2H, s), 3.32 (2H, d), 2.40 (2H, q), 2.34 (3H, s), 2.31 (3H, s), 2.19 (3H, s), 1.06 (3H, t), 1.05 (6H, s).

Compound of the present invention 97: ¹H-NMR (CDCl₃) δ: 7.60-7.51 (1H, m), 7.17 (1H, d), 6.59 (1H, s), 6.54 (1H, s), 4.29 (2H, s), 3.32 (2H, d), 2.49 (3H, s), 2.46 (3H, s), 2.40 (2H, q), 2.19 (3H, s), 1.07 (3H, t), 1.06 (6H, s).

Compound of the present invention 98: ¹H-NMR (CDCl₃) δ:7.58-7.48 (1H, m), 7.10 (1H, d), 6.58 (1H, s), 6.53 (1H, s), 4.28 (2H, s), 3.32 (2H, d), 2.40 (2H, q), 2.39 (3H, s), 2.37 (3H, s), 2.23 (3H, s), 2.19 (3H, s), 1.07 (3H, t), 1.06 (6H, s).

Compound of the present invention 99: ¹H-NMR (CDCl₃) δ:7.52-7.40 (1H, m), 7.49 (1H, s), 7.30 (1H, s), 6.59 (1H, s), 6.50 (1H, s), 4.29 (2H, s), 3.41 (2H, d), 2.46 (3H, s), 2.43 (2H, q), 2.32 (3H, s), 1.82-1.72 (2H, m), 1.70-1.59 (4H, m), 1.78-1.37 (2H, m), 1.06 (3H, t).

Compound of the present invention 100: ¹H-NMR (CDCl₃) δ:7.51-7.39 (2H, m), 7.35 (1H, t), 7.23 (1H, d), 6.61 (1H, s), 6.54 (1H, s), 4.31 (2H, s), 3.42 (2H, d), 2.24 (6H, s), 1.88-1.73 (2H, m), 1.70-1.50 (4H, m), 1.44-1.29 (2H, m).

Compound of the present invention 101: ¹H-NMR (CDCl₃) δ:7.52-7.41 (1H, m), 7.37 (1H, d), 6.98 (1H, s), 6.85 (1H, dd), 6.59 (1H, s), 6.51 (1H, s), 4.28 (2H, s), 3.84 (3H, s), 3.41 (2H, d), 2.43 (2H, q), 2.23 (3H, s), 1.86-1.72 (2H, m), 1.69-1.54 (4H, m), 1.48-1.37 (2H, m), 1.06 (3H, t).

Compound of the present invention 102: ¹H-NMR (CDCl₃) δ:7.49-7.37 (1H, m), 7.26 (1H, s), 7.21 (1H, s), 6.57 (1H, s), 6.47 (1H, s), 4.28 (2H, s), 3.42 (2H, d), 2.34 (3H, s), 2.32 (3H, s), 2.23 (6H, s), 1.85-1.75 (2H, m), 1.70-1.54 (4H, m), 1.42-1.31 (2H, m).

Compound of the present invention 103: ¹H-NMR (CDCl₃) δ:7.50-7.38 (1H, m), 7.26 (1H, s), 7.21 (1H, s), 6.57 (1H, s), 6.47 (1H, s), 4.27 (2H, s), 3.42 (2H, d), 2.44 (2H, q), 2.34 (3H, s), 2.32 (3H, s), 2.23 (3H, s), 1.84-1.72 (2H, m), 1.70-1.54 (4H, m), 1.50-1.36 (2H, m), 1.06 (3H, t).

Compound of the present invention 104: ¹H-NMR (CDCl₃) δ: 7.50-7.38 (1H, m), 7.36 (1H, d), 7.32 (1H, s), 7.10 (1H, d), 6.59 (1H, s), 6.52 (1H, s), 4.30 (2H, s), 3.42 (2H, d), 2.72 (2H, q), 2.23 (6H, s), 1.87-1.74 (2H, m), 1.70-1.52 (4H, m), 1.42-1.31 (2H, m), 1.26 (3H, t).

Compound of the present invention 105: ¹H-NMR (CDCl₃) δ:7.48-7.38 (1H, m), 7.36 (1H, s), 7.34 (1H, d), 7.14 (1H, dd), 6.59 (1H, s), 6.53 (1H, s), 4.30 (2H, s), 3.42 (2H, d), 3.05-2.94 (1H, m), 2.23 (6H, s), 1.86-1.74 (2H, m), 1.68-1.53 (4H, m), 1.42-1.32 (2H, m), 1.28 (6H, d).

Compound of the present invention 106: ¹H-NMR (CDCl₃) δ: 7.56 (1H, d), 7.48-7.38 (1H, m), 7.29 (1H, d), 7.22 (1H, t), 7.11 (1H, t), 6.42 (1H, s), 6.42 (1H, s), 4.31 (2H, s), 3.67 (3H, s), 3.40 (2H, d), 2.23 (6H, s), 1.85-1.75 (2H, m), 1.67-1.54 (4H, m), 1.42-1.30 (2H, m).

Compound of the present invention 107: ¹H-NMR (CDCl₃) δ:7.71 (1H, s), 7.60 (2H, d), 7.52-7.41 (5H, m), 7.34 (1H, t), 6.63 (1H, s), 6.63 (1H, s), 4.34 (2H, s), 3.42 (2H, d), 2.24 (6H, s), 1.87-1.75 (2H, m), 1.72-1.54 (4H, m), 1.43-1.32 (2H, m).

Compound of the present invention 108: ¹H-NMR (CDCl₃) δ:7.48-7.40 (1H, m), 7.23 (1H, s), 6.92 (1H, s), 6.58 (1H, s), 6.45 (1H, s), 4.26 (2H, s), 3.86 (3H, s), 3.42 (2H, d), 2.27 (3H, s), 2.23 (6H, s), 1.86-1.75 (2H, m), 1.71-1.54 (4H, m), 1.42-1.32 (2H, m).

Compound of the present invention 109: ¹H-NMR (CDCl₃) δ: 7.50-7.40 (2H, m), 7.34 (1H, s), 6.59 (1H, s), 6.49 (1H, s), 4.29 (2H, s), 3.42 (2H, d), 2.43 (3H, s), 2.23 (6H, s), 1.85-1.75 (2H, m), 1.72-1.54 (4H, m), 1.42-1.32 (2H, m).

Compound of the present invention 110: ¹H-NMR (CDCl₃) δ:7.48-7.39 (1H, m), 7.10 (1H, s), 6.58 (1H, s), 6.52 (1H, s), 4.28 (2H, s), 3.42 (2H, d), 2.39 (3H, s), 2.37 (3H, s), 2.23 (6H, s), 2.23 (3H, s), 1.86-1.75 (2H, m), 1.71-1.55 (4H, m), 1.43-1.32 (2H, m).

Compound of the present invention 111: ¹H-NMR (CDCl₃) δ: 7.59 (1H, s), 7.56 (1H, s), 7.49-7.41 (1H, m), 6.61 (1H, s), 6.53 (1H, s), 4.31 (2H, s), 3.42 (2H, d), 2.23 (6H, s), 1.86-1.72 (2H, m), 1.70-1.56 (4H, m), 1.42-1.32 (2H, m).

Compound of the present invention 112: ¹H-NMR (CDCl₃) δ:7.53 (1H, d), 7.46-7.37 (1H, m), 6.90 (1H, s), 6.66 (1H, d), 6.42 (1H, s), 6.00 (2H, s), 4.34 (2H, s), 3.39 (2H, d), 2.22 (6H, s), 1.84-1.72 (2H, m), 1.67-1.56 (4H, m), 1.41-1.30 (2H, m).

Compound of the present invention 113: ¹H-NMR (CDCl₃) δ: 7.49-7.40 (1H, m), 7.17 (1H, s), 6.60 (1H, s), 6.53 (1H, s), 4.29 (2H, s), 3.42 (2H, d), 2.49 (3H, s), 2.46 (3H, s), 2.23 (6H, s), 1.85-1.75 (2H, m), 1.70-1.59 (4H, m), 1.42-1.32 (2H, m).

Compound of the present invention 114: ¹H-NMR (CDCl₃) δ:7.51-7.41 (1H, m), 7.10 (1H, s), 6.58 (1H, s), 6.53 (1H, s), 4.28 (2H, s), 3.42 (2H, d), 2.44 (2H, q), 2.39 (3H, s), 2.37 (3H, s), 2.23 (3H, s), 2.23 (3H, s), 1.82-1.72 (2H, m), 1.71-1.56 (4H, m), 1.49-1.38 (2H, m), 1.06 (3H, t).

Compound of the present invention 115: ¹H-NMR (CDCl₃) δ:7.50-7.40 (2H, m), 7.34 (1H, s), 6.59 (1H, s), 6.49 (1H, s), 4.29 (2H, s), 3.42 (2H, d), 2.43 (3H, s), 2.23 (6H, s), 1.85-1.75 (2H, m), 1.72-1.54 (4H, m), 1.42-1.32 (2H, m).

Compound of the present invention 116: ¹H-NMR (CDCl₃) δ: 7.51-7.42 (1H, m), 7.17 (1H, s), 6.60 (1H, s), 6.54 (1H, s), 4.29 (2H, s), 3.42 (2H, d), 2.49 (3H, s), 2.46 (3H, s), 2.44 (2H, q), 2.23 (3H, s), 1.83-1.72 (2H, m), 1.71-1.51 (4H, m), 1.48-1.37 (2H, m), 1.06 (3H, t).

Compound of the present invention 117: ¹H-NMR (CDCl₃) δ:7.49-7.40 (1H, m), 7.26 (1H, s), 7.24 (1H, s), 6.58 (1H, s), 6.47 (1H, s), 4.28 (2H, s), 3.42 (2H, d), 2.70 (2H, q), 2.36 (3H, s), 2.23 (6H, s), 1.85-1.75 (2H, m), 1.69-1.57 (4H, m), 1.42-1.32 (2H, m), 1.25 (3H, t).

Compound of the present invention 118: ¹H-NMR (CDCl₃) δ: 7.75-7.65 (1H, m), 7.31 (1H, d), 7.30 (1H, s), 7.07 (1H, d), 6.59 (1H, s), 6.51 (1H, s), 4.29 (2H, s), 3.53 (2H, d), 3.52 (3H, s), 2.60 (3H, s), 2.42 (3H, s), 1.82-1.58 (6H, m), 1.54-1.42 (2H, m).

Compound of the present invention 119: ¹H-NMR (CDCl₃) δ:7.49-7.39 (1H, m), 7.30 (1H, d), 7.29 (1H, s), 7.07 (1H, d), 6.59 (1H, s), 6.50 (1H, s), 4.29 (2H, s), 3.36 (2H, d), 2.55 (2H, q), 2.42 (3H, s), 1.77-1.49 (8H, m), 1.09 (3H, t).

Compound of the present invention 120: ¹H-NMR (CDCl₃) δ:7.51 (1H, d), 7.47-7.39 (1H, m), 7.21 (1H, s), 7.12 (1H, d), 6.56 (1H, s), 4.39 (2H, s), 3.41 (2H, d), 2.47 (3H, s), 2.38 (3H, s), 2.23 (6H, s), 1.86-1.74 (2H, m), 1.70-1.54 (4H, m), 1.41-1.32 (2H, m).

Compound of the present invention 121: ¹H-NMR (CDCl₃) δ: 7.53 (1H, s), 7.48-7.38 (2H, m), 7.04 (1H, s), 6.55 (1H, s), 4.36 (2H, s), 3.41 (2H, d), 2.35 (3H, s), 2.34 (3H, s), 2.23 (6H, s), 1.86-1.73 (2H, m), 1.72-1.54 (4H, m), 1.43-1.31 (2H, m).

Compound of the present invention 122: ¹H-NMR (CDCl₃) δ:7.58-7.46 (1H, m), 7.10 (1H, s), 6.58 (1H, d), 6.52 (1H, s), 4.28 (2H, s), 3.34 (2H, d), 2.94-2.84 (1H, m), 2.39 (3H, s), 2.37 (3H, s), 2.23 (3H, s), 1.74-1.49 (8H, m), 1.07 (6H, d).

Compound of the present invention 170: ¹H-NMR (CDCl₃) δ: 7.54-7.43 (1H, m), 7.39-7.28 (5H, m), 7.26-7.20 (2H, m), 6.58 (1H, d), 6.48 (1H, s), 4.28 (2H, s), 3.70 (2H, s), 3.44 (2H, d), 2.35 (3H, s), 2.32 (3H, s), 1.76-1.32 (9H, m).

Compound of the present invention 173: ¹H-NMR (CDCl₃) δ:7.39-7.28 (1H, m), 7.26 (1H, s), 7.21 (1H, s), 6.58 (1H, d), 6.47 (1H, s), 4.27 (2H, s), 3.35 (2H, d), 2.34 (6H, s); 2.31 (3H, s), 1.99-1.77 (1H, m), 1.13 (6H, s).

Compound of the present invention 174: ¹H-NMR (CDCl₃) δ:7.48 (1H, s), 7.40-7.32 (1H, m), 7.30 (1H, s), 6.59 (1H, d), 6.49 (1H, s), 4.29 (2H, s), 3.38 (2H, d), 2.46 (3H, s), 2.30 (3H, s), 1.73-1.29 (9H, m).

### Production Example 5

500 mg of ethyl iodide was added to a mixture of 240 mg of Compound of the present invention 46, 450 mg of potassium carbonate, and 10 mL of acetonitrile, and the mixture was stirred under heat-reflux for 2 hours. Water was added to the resulting mixture, and the mixture was extracted with MTBE. The resulting organic layer was dried over anhydrous sodium sulfate and concentrated under reduced pressure. The resulting residue was subjected to silica gel chromatography (chloroform:methanol=10:1) to provide 50 mg of Compound of the present invention 123 represented by the following formula.

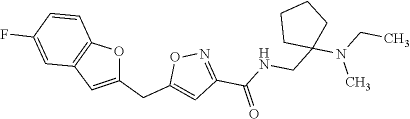 [SMILES: CCN(C)C1(CNC(=O)c2cc(Cc3cc4cc(F)ccc4o3)on2)CCCC1]

Compound of the present invention 123: ¹H-NMR (CDCl₃) δ: 7.54-7.41 (1H, m), 7.35 (1H, dd), 7.17 (1H, dd), 6.99 (1H, td), 6.60 (1H, s), 6.56 (1H, s), 4.31 (2H, s), 3.42 (2H, d), 2.44 (2H, q), 2.23 (3H, s), 1.84-1.72 (2H, m), 1.70-1.54 (4H, m), 1.83-1.35 (2H, m), 1.06 (3H, t).

### Production Example 5-1

The compound produced in accordance with Production example 5 and physical properties thereof are shown below.

A compound represented by formula (B-5),

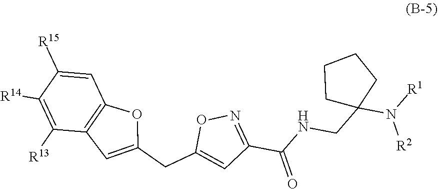

wherein the combination of R¹, R², R¹³, R¹⁴, and R¹⁵ is any one of the combinations shown in [Table B-6],

TABLE B-6

| Compound | | | | | |
|---|---|---|---|---|---|
| of the | | | | | |
| present | | | | | |
| invention | R¹ | R² | R¹³ | R¹⁴ | R¹⁵ |
| 124 | Pr | Me | H | Me | Me |
| 125 | CH₂—Ph | Et | H | Me | Me |
| 126 | CH₂CN | Et | H | Me | Me |
| 127 | CH₂—(4-CF₃—Ph) | Et | H | Me | Me |
| 128 | CH₂CO₂Et | Et | H | Me | Me |
| 129 | CH₂CN | H | H | Me | Me |
| 130 | CH₂—(4-OCH₃—Ph) | Et | H | Me | Me |
| 131 | CH₂CH═CMe₂ | Et | H | Me | Me |
| 132 | CH₂—Ph | Et | Me | Cl | Me |
| 133 | CH₂C(O)OEt | Et | Me | Cl | Me |
| 134 | CH₂—[3,4,5—(OCH₃)₃—Ph] | Et | H | Me | Me |
| 135 | CH₂C(O)O(t-Bu) | Et | H | Me | Me |
| 136 | CH₂C(O)OMe | Et | H | Me | Me |
| 137 | CH₂—(4-CH₃—Ph) | Et | H | Me | Me |
| 138 | i-Pr | Me | H | Me | Me |
| 139 | i-Pr | Me | Me | Cl | Me |
| 140 | CH₂CH═CCl₂ | Et | H | Me | Me |
| 141 | CH₂CH═CMe₂ | Et | Me | Cl | Me |
| 142 | CH₂CH═CH₂ | Et | H | Me | Me |
| 143 | CH₂C≡CH | Et | H | Me | Me |
| 144 | CH₂CH═C(H)Me | Et | H | Me | Me |
| 166 | CH₂C≡CH | CH₂C≡CH | H | Me | Me |
| 167 | CH₂C≡CH | H | H | Me | Me |
| 169 | CH₂C≡CH | i-Pr | H | Me | Me |
| 171 | CH₂C≡CH | Bn | H | Me | Me |
| 172 | CH₂-3-CO₂Et-isoxazol-5-yl | Et | H | Me | Me |

Compound of the present invention 124: ¹H-NMR (CDCl₃) δ: 7.51-7.44 (1H, m), 7.26 (1H, s), 7.21 (1H, s), 6.57 (1H, s), 6.47 (1H, s), 4.27 (2H, s), 3.41 (2H, d), 2.36-2.29 (2H, m), 2.34 (3H, s), 2.31 (3H, s), 2.21 (3H, s), 1.81-1.71 (2H, m), 1.69-1.58 (4H, m), 1.52-1.37 (4H, m), 0.89 (3H, t).

Compound of the present invention 125: ¹H-NMR (CDCl₃) δ:7.61-7.48 (1H, m), 7.40 (2H, t), 7.30 (2H, t), 7.27 (1H, s), 7.22 (1H, s), 7.18 (1H, t), 6.57 (1H, s), 6.49 (1H, s), 4.30 (2H, s), 3.74 (2H, s), 3.44 (2H, d), 2.66 (2H, q), 2.35 (3H, s), 2.32 (3H, s), 1.85-1.74 (2H, m), 1.73-1.58 (4H, m), 1.55-1.46 (2H, m), 0.95 (3H, t).

Compound of the present invention 126: ¹H-NMR (CDCl₃) δ:7.26 (1H, s), 7.21 (1H, s), 7.17-7.05 (1H, m), 6.57 (1H, s), 6.47 (1H, s), 4.28 (2H, s), 3.69 (2H, s), 3.46 (2H, d), 2.79 (2H, q), 2.35 (3H, s), 2.32 (3H, s), 1.94-1.51 (8H, m), 1.12 (3H, t).

Compound of the present invention 127: ¹H-NMR (CDCl₃) δ:7.58-7.44 (5H, m), 7.27 (1H, s), 7.22 (1H, s), 6.58 (1H, s), 6.50 (1H, s), 4.30 (2H, s), 3.79 (2H, s), 3.45 (2H, d), 2.68 (2H, q), 2.35 (3H, s), 2.32 (3H, s), 1.81-1.58 (6H, m), 1.56-1.45 (2H, m), 0.95 (3H, t).

Compound of the present invention 128: ¹H-NMR (CDCl₃) δ: 8.13-8.03 (1H, m), 7.26 (1H, s), 7.21 (1H, s), 6.59 (1H, s), 6.47 (1H, s), 4.27 (2H, s), 4.18 (2H, q), 3.40 (2H, s), 3.38 (2H, d), 2.73 (2H, q), 2.34 (3H, s), 2.32 (3H, s), 1.72-1.62 (6H, m), 1.58-1.48 (2H, m), 1.24 (3H, t), 1.03 (3H, t).

Compound of the present invention 129: ¹H-NMR (CDCl₃) δ:7.26 (1H, s), 7.21 (1H, s), 7.15-7.06 (1H, m), 6.57 (1H, s), 6.48 (1H, s), 4.28 (2H, s), 3.57 (2H, s), 3.45 (2H, d), 2.34 (3H, s), 2.32 (3H, s), 1.85-1.60 (8H, m), 1.43-1.33 (1H, m).

Compound of the present invention 130: ¹H-NMR (CDCl₃) δ:7.60-7.51 (1H, m), 7.30 (2H, d), 7.26 (1H, s), 7.22 (1H, s), 6.84 (2H, d), 6.57 (1H, s), 6.49 (1H, s), 4.29 (2H, s), 3.77 (3H, s), 3.66 (2H, s), 3.43 (2H, d), 2.64 (2H, q), 2.35 (3H, s), 2.32 (3H, s), 1.84-1.58 (6H, m), 1.55-1.44 (2H, m), 0.93 (3H, t).

Compound of the present invention 131: ¹H-NMR (CDCl₃) δ:7.56-7.45 (1H, m), 7.26 (1H, s), 7.21 (1H, s), 6.57 (1H, s), 6.47 (1H, s), 5.26-5.19 (1H, m), 4.28 (2H, s), 3.36 (2H, d), 3.15 (2H, d), 2.57 (2H, q), 2.34 (3H, s), 2.32 (3H, s), 1.81-1.59 (12H, m), 1.53-1.42 (2H, m), 1.02 (3H, t).

Compound of the present invention 132: ¹H-NMR (CDCl₃) δ:7.63-7.52 (1H, m), 7.42-7.38 (2H, m), 7.33-7.27 (2H, m), 7.21-7.15 (2H, m), 6.59 (1H, s), 6.55 (1H, s), 4.31 (2H, s), 3.74 (2H, s), 3.44 (2H, d), 2.66 (2H, q), 2.50 (3H, s), 2.46 (3H, s), 1.86-1.74 (2H, m), 1.73-1.57 (4H, m), 1.55-1.45 (2H, m), 0.95 (3H, t).

Compound of the present invention 133: ¹H-NMR (CDCl₃) δ:8.16-8.02 (1H, m), 7.17 (1H, s), 6.61 (1H, s), 6.54 (1H, s), 4.29 (2H, s), 4.17 (2H, q), 3.40 (2H, s), 3.38 (2H, d), 2.73 (2H, q), 2.49 (3H, s), 2.46 (3H, s), 1.72-1.48 (8H, m), 1.25 (3H, t), 1.03 (3H, t).

Compound of the present invention 134: ¹H-NMR (CDCl₃) δ: 7.65-7.59 (1H, m), 7.26 (1H, d), 7.21 (1H, s), 6.64 (2H, s), 6.57 (1H, s), 6.47 (1H, s), 4.27 (2H, s), 3.88 (6H, s), 3.81 (3H, s), 3.67 (2H, s), 3.44 (2H, d), 2.65 (2H, q), 2.35 (3H, s), 2.32 (3H, s), 1.85-1.58 (6H, m), 1.56-1.48 (2H, m), 0.99 (3H, t).

Compound of the present invention 135: ¹H-NMR (CDCl₃) δ:8.03-7.95 (1H, m), 7.25 (1H, s), 7.21 (1H, s), 6.59 (1H, s), 6.47 (1H, s), 4.27 (2H, s), 3.37 (2H, d), 3.29 (2H, s), 2.72 (2H, q), 2.34 (3H, s), 2.32 (3H, s), 1.74-1.51 (8H, m), 1.44 (9H, s), 1.03 (3H, t).

Compound of the present invention 136: ¹H-NMR (CDCl₃) δ: 8.12-8.03 (1H, m), 7.26 (1H, s), 7.21 (1H, s), 6.59 (1H, s), 6.47 (1H, s), 4.27 (2H, s), 3.73 (3H, s), 3.42 (2H, s), 3.37 (2H, d), 2.72 (2H, q), 2.34 (3H, s), 2.30 (3H, s), 1.74-1.48 (8H, m), 1.02 (3H, t).

Compound of the present invention 137: ¹H-NMR (CDCl₃) δ: 7.62-7.54 (1H, m), 7.28 (2H, d), 7.27 (1H, s), 7.22 (1H, s), 7.11 (2H, s), 6.57 (1H, s), 6.49 (1H, s), 4.30 (2H, s), 3.69 (2H, s), 3.43 (2H, d), 2.64 (2H, q), 2.35 (3H, s), 2.32 (3H, s), 2.30 (3H, s), 1.84-1.59 (6H, m), 1.56-1.45 (2H, m), 0.94 (3H, t).

Compound of the present invention 138: ¹H-NMR (CDCl₃) δ:7.50-7.40 (1H, m), 7.25 (1H, s), 7.21 (1H, s), 6.57 (1H, s), 6.47 (1H, s), 4.27 (2H, s), 3.37 (2H, d), 3.29-3.18 (1H, m), 2.34 (3H, s), 2.31 (3H, s), 2.21 (3H, s), 1.89-1.78 (2H, m), 1.74-1.54 (4H, m), 1.49-1.37 (2H, m), 1.03 (6H, d).

Compound of the present invention 139: ¹H-NMR (CDCl₃) δ:7.51-7.44 (1H, m), 7.17 (1H, s), 6.59 (1H, s), 6.53 (1H, s), 4.29 (2H, s), 3.37 (2H, d), 3.29-3.18 (1H, m), 2.49 (3H, s), 2.46 (3H, s), 2.21 (3H, s), 1.88-1.79 (2H, m), 1.72-1.56 (4H, m), 1.48-1.38 (2H, m), 1.04 (6H, d).

Compound of the present invention 140: ¹H-NMR (CDCl₃) δ:7.36-7.28 (1H, m), 7.26 (1H, s), 7.21 (1H, s), 6.58 (1H, s), 6.48 (1H, s), 5.97 (1H, t), 4.28 (2H, s), 3.39 (2H, d), 3.35 (2H, d), 2.62 (2H, q), 2.34 (3H, s), 2.32 (3H, s), 1.76-1.58 (6H, m), 1.54-1.45 (2H, m), 1.06 (3H, t).

Compound of the present invention 141: ¹H-NMR (CDCl₃) δ: 7.59-7.49 (1H, m), 7.17 (1H, s), 6.60 (1H, s), 6.54 (1H, s), 5.26-5.19 (1H, m), 4.29 (2H, s), 3.36 (2H, d), 3.15 (2H, d), 2.57 (2H, q), 2.49 (3H, s), 2.46 (3H, s), 1.86-1.57 (12H, m), 1.54-1.42 (2H, m), 1.02 (3H, t).

Compound of the present invention 142: ¹H-NMR (CDCl₃) δ:7.54-7.45 (1H, m), 7.26 (1H, s), 7.21 (1H, s), 6.58 (1H, s), 6.47 (1H, s), 5.94-5.83 (1H, m), 5.25-5.18 (1H, m), 5.07-5.02 (1H, m), 4.27 (2H, s), 3.38 (2H, d), 3.18 (2H, d), 2.61 (2H, q), 2.34 (3H, s), 2.32 (3H, s), 1.79-1.56 (6H, m), 1.53-1.44 (2H, m), 1.02 (3H, t).

Compound of the present invention 143: ¹H-NMR (CDCl₃) δ:7.44-7.37 (1H, m), 7.26 (1H, s), 7.21 (1H, s), 6.57 (1H, s), 6.47 (1H, s), 4.27 (2H, s), 3.55 (2H, d), 3.46 (2H, d), 2.77 (2H, q), 2.34 (3H, s), 2.31 (3H, s), 2.23 (1H, t), 1.90-1.80 (2H, m), 1.76-1.61 (4H, m), 1.59-1.51 (2H, m), 1.07 (3H, t).

Compound of the present invention 144: ¹H-NMR (CDCl₃) δ:7.55-7.44 (1H, m), 7.26 (1H, s), 7.21 (1H, s), 6.58 (1H, s), 6.47 (1H, s), 5.63-5.41 (2H, m), 4.28 (2H, s), 3.37 (2H, d), 3.12 (2H, d), 2.59 (2H, q), 2.34 (3H, s), 2.32 (3H, s), 1.83-1.58 (9H, m), 1.53-1.43 (2H, m), 1.01 (3H, t).

Compound of the present invention 166: ¹H-NMR (CDCl₃) δ: 7.39-7.31 (1H, m), 7.26 (1H, s), 7.21 (1H, s), 6.57 (1H, s), 6.47 (1H, s), 4.27 (2H, s), 3.69 (4H, d), 3.50 (2H, d), 2.34 (3H, s), 2.32 (3H, s), 2.27 (2H, t), 1.93-1.82 (2H, m), 1.79-1.64 (4H, m), 1.63-1.54 (2H, m).

Compound of the present invention 167: ¹H-NMR (CDCl₃) δ:7.43-7.33 (1H, m), 7.26 (1H, s), 7.21 (1H, s), 6.57 (1H, s), 6.47 (1H, s), 4.27 (2H, s), 3.44 (2H, d), 3.38 (2H, d), 2.34 (3H, s), 2.31 (3H, s), 2.24 (1H, t), 1.79-1.52 (9H, m).

Compound of the present invention 169: ¹H-NMR (CDCl₃) δ:7.43-7.34 (1H, m), 7.25 (1H, s), 7.21 (1H, s), 6.57 (1H, s), 6.47 (1H, s), 4.27 (2H, s), 3.46 (2H, d), 3.42 (2H, d), 3.35-3.24 (1H, m), 2.34 (3H, s), 2.32 (3H, s), 2.18 (1H, t), 2.02-1.90 (2H, m), 1.77-1.53 (6H, m), 1.14 (6H, d).

Compound of the present invention 171: ¹H-NMR (CDCl₃) δ:7.54-7.47 (1H, m), 7.38-7.29 (4H, m), 7.27-7.20 (3H, m), 6.59 (1H, d), 6.48 (1H, s), 4.28 (2H, s), 3.93 (2H, s), 3.60 (2H, d), 3.40 (2H, d), 2.34 (3H, s), 2.32 (3H, s), 2.31 (1H, t), 2.02-1.91 (2H, m), 1.83-1.62 (6H, m).

Compound of the present invention 172: ¹H-NMR (CDCl₃) δ:7.41-7.33 (1H, m), 7.26 (1H, s), 7.21 (1H, s), 6.74 (1H, s), 6.59 (1H, s), 6.49 (1H, s), 4.41 (2H, q), 4.29 (2H, s), 3.91 (2H, s), 3.46 (2H, d), 2.73 (2H, q), 2.35 (3H, s), 2.32 (3H, s), 1.76-1.50 (8H, m), 1.39 (3H, t), 1.03 (3H, t).

### Production Example 5-2

The compound produced in accordance with Production example 5 and physical properties thereof are shown below.

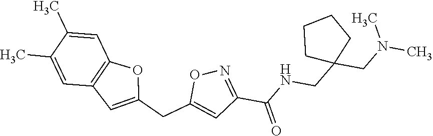

Compound of the present invention 145: ¹H-NMR (CDCl₃) δ:9.30-9.23 (1H, m), 7.25 (1H, s), 7.21 (1H, s), 6.56 (1H, s), 6.46 (1H, s), 4.26 (2H, s), 3.38 (2H, d), 2.42-2.26 (14H, m), 1.76-1.53 (6H, m), 1.39-1.28 (2H, m).

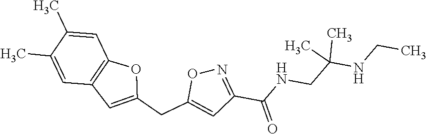 [SMILES: CCNC(C)(C)CNC(=O)c1cc(Cc2cc3cc(C)c(C)cc3o2)on1]

Compound of the present invention 175: ¹H-NMR (CDCl₃) δ: 7.36-7.27 (1H, m), 7.26 (1H, s), 7.21 (1H, s), 6.58 (1H, s), 6.47 (1H, s), 4.28 (2H, s), 3.30 (2H, d), 2.57 (2H, q), 2.35 (3H, s), 2.32 (3H, s), 1.28-1.22 (1H, m), 1.12 (6H, s), 1.09 (3H, t).

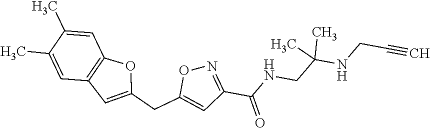 [SMILES: C#CCNC(C)(C)CNC(=O)c1cc(Cc2cc3cc(C)c(C)cc3o2)on1]

Compound of the present invention 176: ¹H-NMR (CDCl₃) δ: 7.34-7.27 (1H, m), 7.26 (1H, s), 7.21 (1H, s), 6.58 (1H, s), 6.47 (1H, s), 4.27 (2H, s), 3.40 (2H, d), 3.36 (2H, d), 2.34 (3H, s), 2.31 (3H, s), 2.23 (1H, t), 1.29-1.22 (1H, m), 1.15 (6H, s).

### Production Example 6

2 mL of trifluoroacetic acid was added to a mixture of 1.08 g of Intermediate 78 and 20 mL of chloroform, and the mixture was stirred at room temperature for 2 hours and 30 minutes. An aqueous sodium bicarbonate solution was added to the resulting mixture, which was then extracted with chloroform. The resulting organic layer was dried over anhydrous sodium sulfate and concentrated under reduced pressure. The resulting residue was subjected to silica gel chromatography (chloroform:methanol=10:1) to provide 880 mg of Compound of the present invention 146 represented by the following formula.

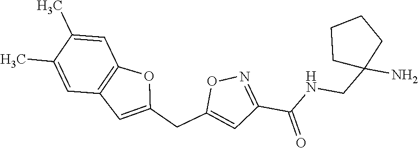 [SMILES: Cc1cc2cc(Cc3cc(C(=O)NCC4(N)CCCC4)no3)oc2cc1C]

Compound of the present invention 146: ¹H-NMR (CDCl₃) δ: 7.36-7.28 (1H, m), 7.26 (1H, s), 7.21 (1H, s), 6.58 (1H, s), 6.47 (1H, s), 4.28 (2H, s), 3.40 (2H, d), 2.34 (3H, s), 2.32 (3H, s), 1.85-1.59 (4H, m), 1.53-1.41 (6H, m).

### Production Example 6-1

The compound produced in accordance with Production example 6 and physical properties thereof are shown below.

A compound represented by formula (B-6),

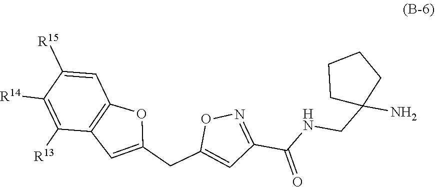

in which the combination of R¹³, R¹⁴, and R¹⁵ is any one of the combinations shown in [Table B-7],

TABLE B-7

| Compound of the | | | |
|---|---|---|---|
| present invention | R¹³ | R¹⁴ | R¹⁵ |
| 147 | H | H | H |
| 148 | H | H | Me |
| 149 | Me | Cl | Me |

Compound of the present invention 147: ¹H-NMR (CDCl₃) δ:7.52 (1H, d), 7.44 (1H, d), 7.38-7.19 (3H, m), 6.61 (1H, s), 6.59 (1H, s), 4.32 (2H, s), 3.40 (2H, d), 1.86-1.59 (6H, m), 1.54-1.43 (2H, m), 1.41-1.24 (2H, m).

Compound of the present invention 148: ¹H-NMR (CDCl₃) δ:7.38 (1H, d), 7.36-7.29 (1H, m), 7.24 (1H, s), 7.04 (1H, d), 6.59 (1H, s), 6.53 (1H, s), 4.29 (2H, s), 3.41 (2H, d), 2.45 (3H, s), 1.86-1.59 (6H, m), 1.54-1.36 (4H, m).

Compound of the present invention 149: ¹H-NMR (CDCl₃) δ:7.38-7.29 (1H, m), 7.17 (1H, s), 6.60 (1H, s), 6.53 (1H, s), 4.29 (2H, s), 3.40 (2H, d), 2.49 (3H, s), 2.46 (3H, s), 1.85-1.60 (6H, m), 1.53-1.29 (4H, m).

### Production Example 7

To 80 mg of Compound of the present invention 103, 5 mL of hydrogen chloride (4 mol/L cyclopentyl methyl ether solution) was added, and the mixture was stirred at room temperature for 1 hour and 30 minutes. The resulting mixture was concentrated under reduced pressure to provide 58 mg of Compound of the present invention 150 represented by the following formula.

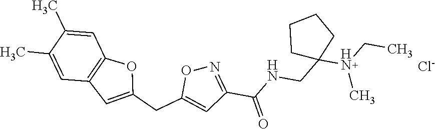

Compound of the present invention 150: ¹H-NMR (CDCl₃) δ:12.05-11.89 (1H, m), 7.88-7.66 (1H, m), 7.26 (1H, s), 7.22 (1H, s), 6.71 (1H, s), 6.50 (1H, s), 4.29 (2H, s), 3.82-3.63 (2H, m), 3.45-3.31 (1H, m), 3.14-3.01 (1H, m), 2.81 (3H, s), 2.52-2.36 (2H, m), 2.35 (3H, s), 2.32 (3H, s), 2.08-1.68 (6H, m), 1.55 (3H, t).

### Production Example 7-1

The compound produced in accordance with Production example 7 and physical properties thereof are shown below.

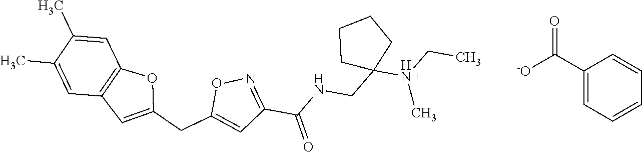

Compound of the present invention 151: ¹H-NMR (CDCl₃) δ: 8.28-8.18 (1H, m), 8.07 (2H, d), 7.48 (1H, t), 7.39 (2H, t), 7.24 (1H, s), 7.20 (1H, s), 6.57 (1H, s), 6.41 (1H, s), 6.24-5.58 (1H, m), 4.20 (2H, s), 3.62 (2H, d), 2.80 (2H, q), 2.53 (3H, s), 2.34 (3H, s), 2.31 (3H, s), 2.09-1.92 (2H, m), 1.82-1.59 (6H, m), 1.24 (3H, t).

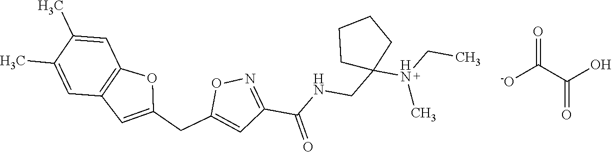

Compound of the present invention 152: ¹H-NMR (CDCl₃) δ: 11.94-11.63 (1H, m), 8.22-7.98 (1H, m), 7.26 (1H, s), 7.21 (1H, s), 6.57 (1H, s), 6.49 (1H, s), 4.27 (2H, s), 3.98-3.67 (3H, m), 3.54-3.40 (1H, m), 3.00-2.90 (1H, m), 2.88 (3H, s), 2.34 (3H, s), 2.32 (3H, s), 2.28-2.12 (2H, m), 1.97-1.70 (6H, m), 1.39 (3H, t).

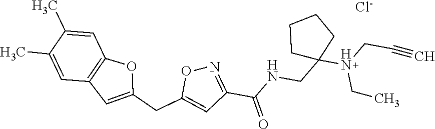

Compound of the present invention 168: ¹H-NMR (CDCl₃) δ: 8.01-7.90 (1H, m), 7.26 (1H, s), 7.21 (1H, s), 6.70 (1H, s), 6.49 (1H, s), 4.29 (2H, s), 4.26-4.21 (1H, m), 4.00-3.74 (3H, m), 3.55-3.33 (2H, m), 2.81 (1H, t), 2.59-2.47 (1H, m), 2.42-2.37 (1H, m), 2.34 (3H, s), 2.32 (3H, s), 2.04-1.76 (6H, m), 1.61 (3H, t).

### Production Example 8

0.05 mL of acetyl chloride was added to a mixture of 150 mg of Compound of the present invention 55, 0.16 mL of triethylamine, and 5 mL of chloroform, and the mixture was stirred at room temperature for 10 minutes. The resulting mixture was concentrated under reduced pressure. The resulting residue was subjected to silica gel chromatography (hexane:ethyl acetate=1:1) to provide 150 mg of Compound of the present invention 153 represented by the following formula.

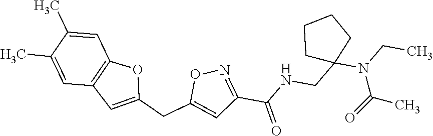 [SMILES: CCN(C(C)=O)C1(CNC(=O)c2cc(Cc3cc4cc(C)c(C)cc4o3)on2)CCCC1]

Compound of the present invention 153: ¹H-NMR (CDCl₃) δ:7.73-7.58 (1H, m), 7.26 (1H, s), 7.21 (1H, s), 6.56 (1H, s), 6.47 (1H, s), 4.27 (2H, s), 3.73 (2H, d), 3.35 (2H, q), 2.34 (3H, s), 2.31 (3H, s), 2.25-2.13 (5H, m), 1.93-1.81 (2H, m), 1.77-1.67 (4H, m), 1.27 (3H, t).

### Production Example 8-1

The compound produced in accordance with Production example 8 and physical properties thereof are shown below.

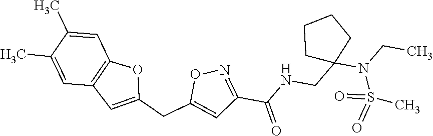 [SMILES: CCN(C1(CNC(=O)c2cc(Cc3cc4cc(C)c(C)cc4o3)on2)CCCC1)S(C)(=O)=O]

Compound of the present invention 154: ¹H-NMR (CDCl₃) δ:7.62-7.52 (1H, m), 7.26 (1H, s), 7.21 (1H, s), 6.56 (1H, s), 6.47 (1H, s), 4.27 (2H, s), 3.66 (2H, d), 3.40 (2H, q), 3.00 (3H, s), 2.34 (3H, s), 2.31 (3H, s), 2.15-2.02 (2H, m), 2.01-1.90 (2H, m), 1.84-1.70 (4H, m), 1.27 (3H, t).

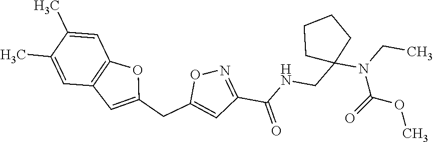 [SMILES: CCN(C(=O)OC)C1(CNC(=O)c2cc(Cc3cc4cc(C)c(C)cc4o3)on2)CCCC1]

Compound of the present invention 155: ¹H-NMR (CDCl₃) δ: 7.39-7.28 (1H, m), 7.26 (1H, s), 7.21 (1H, s), 6.57 (1H, s), 6.47 (1H, s), 4.27 (2H, s), 3.70 (3H, s), 3.68 (2H, d), 3.31 (2H, q), 2.34 (3H, s), 2.32 (3H, s), 2.19-2.05 (2H, m), 1.92-1.81 (2H, m), 1.79-1.64 (4H, m), 1.14 (3H, t).

### Production Example 9

420 mg of sodium triacetoxyborohydride was added to a mixture of 260 mg of Compound of the present invention 55, 60 mg of acetaldehyde, and 10 mL of THF, and the mixture was stirred at room temperature for 3 hours and 20 minutes. An aqueous sodium bicarbonate solution was added to the resulting mixture, which was then extracted with MTBE. The resulting organic layer was dried over anhydrous sodium sulfate and concentrated under reduced pressure. The resulting residue was subjected to silica gel chromatography (chloroform:methanol=10:1) to provide 280 mg of Compound of the present invention 156 represented by the following formula.

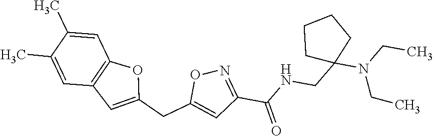 [SMILES: CCN(CC)C1(CNC(=O)c2cc(Cc3cc4cc(C)c(C)cc4o3)on2)CCCC1]

Compound of the present invention 156: ¹H-NMR (CDCl₃) δ: 7.60-7.47 (1H, m), 7.26 (1H, s), 7.21 (1H, s), 6.57 (1H, s), 6.47 (1H, s), 4.27 (2H, s), 3.35 (2H, d), 2.57 (4H, q), 2.34 (3H, s), 2.31 (3H, s), 1.78-1.55 (6H, m), 1.53-1.40 (2H, m), 1.06 (6H, t).

### Production Example 9-1

The compound produced in accordance with Production example 9 and physical properties thereof are shown below.

A compound represented by formula (B-5),

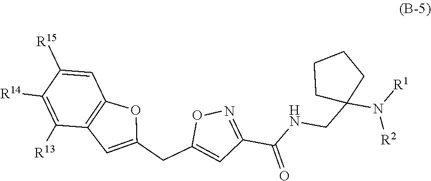

wherein the combination of R¹, R², R¹³, R¹⁴, and R¹⁵ is any one of the combinations shown in [Table B-8].

TABLE B-8

| Compound | | | | | |
|---|---|---|---|---|---|
| of the | | | | | |
| present | | | | | |
| invention | R¹ | R² | R¹³ | R¹⁴ | R¹⁵ |
| 157 | CH₂CH₂OCH₃ | Et | H | Me | Me |
| 158 | CH₂(c-Pr) | Et | H | Me | Me |
| 159 | Et | Et | H | H | Me |
| 160 | i-Pr | Et | H | Me | Me |
| 161 | i-Pr | Et | Me | Cl | Me |

Compound of the present invention 157: ¹H-NMR (CDCl₃) δ:7.81-7.72 (1H, m), 7.26 (1H, s), 7.21 (1H, s), 6.57 (1H, s), 6.47 (1H, s), 4.27 (2H, s), 3.43-3.33 (7H, m), 2.72 (2H, t), 2.62 (2H, q), 2.34 (3H, s), 2.32 (3H, s), 1.76-1.59 (6H, m), 1.56-1.40 (2H, m), 1.05 (3H, t).

Compound of the present invention 158: ¹H-NMR (CDCl₃) δ: 7.64-7.51 (1H, m), 7.26 (1H, s), 7.21 (1H, s), 6.57 (1H, s), 6.47 (1H, s), 4.27 (2H, s), 3.35 (2H, d), 2.68 (2H, q), 2.40 (2H, d), 2.34 (3H, s), 2.31 (3H, s), 1.80-1.58 (6H, m), 1.53-1.42 (2H, m), 1.09 (3H, t), 0.90-0.78 (1H, m), 0.57-0.42 (2H, m), 0.18-0.06 (2H, m).

Compound of the present invention 159: ¹H-NMR (CDCl₃) δ:7.60-7.48 (1H, m), 7.38 (1H, d), 7.24 (1H, s), 7.04 (1H, d), 6.58 (1H, s), 6.53 (1H, s), 4.29 (2H, s), 3.35 (2H, d), 2.57 (4H, q), 2.46 (3H, s), 1.79-1.58 (6H, m), 1.52-1.41 (2H, m), 1.06 (6H, t).

Compound of the present invention 160: ¹H-NMR (CDCl₃) δ: 7.60-7.50 (1H, m), 7.26 (1H, s), 7.21 (1H, s), 6.57 (1H, s), 6.47 (1H, s), 4.27 (2H, s), 3.31 (2H, d), 3.27-3.15 (1H, m), 2.58 (2H, q), 2.34 (3H, s), 2.31 (3H, s), 1.80-1.60 (6H, m), 1.55-1.46 (2H, m), 1.05 (3H, t), 1.05 (6H, d).

Compound of the present invention 161: ¹H-NMR (CDCl₃) δ:7.60-7.52 (1H, m), 7.17 (1H, s), 6.60 (1H, s), 6.54 (1H, s), 4.29 (2H, s), 3.31 (2H, d), 3.26-3.16 (1H, m), 2.58 (2H, q), 2.49 (3H, s), 2.46 (3H, s), 1.80-1.47 (8H, m), 1.05 (3H, t), 1.05 (6H, d).

### Production Example 10

130 mg of sodium triacetoxyborohydride was added to a mixture of 190 mg of Intermediate 75, 60 mg of N-methoxy-N-methylamine hydrochloride, and 10 mL of THF, and the mixture was stirred at room temperature for 3 hours and 30 minutes. An aqueous sodium bicarbonate solution was added to the resulting mixture, which was then extracted with MTBE. The resulting organic layer was dried over anhydrous sodium sulfate and concentrated under reduced pressure. The resulting residue was subjected to silica gel chromatography (hexane:ethyl acetate=5:1) to provide 110 mg of Compound of the present invention 162 represented by the following formula.

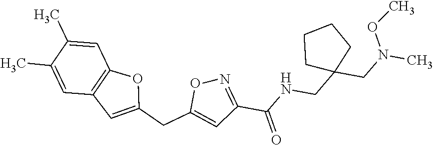

Compound of the present invention 162: ¹H-NMR (CDCl₃) δ:8.15-8.07 (1H, m), 7.26 (1H, s), 7.21 (1H, s), 6.58 (1H, s), 6.47 (1H, s), 4.27 (2H, s), 3.53 (3H, s), 3.41 (2H, d), 2.67 (2H, s), 2.61 (3H, s), 2.34 (3H, s), 2.31 (3H, s), 1.75-1.39 (8H, m).

### Production Example 11

4 mL of sodium hydroxide (1 mol/L aqueous solution) was added to a mixture of 190 mg of Compound of the present invention 128, and 10 mL of THF, and the mixture was stirred at room temperature for 12 hours. Hydrogen chloride (10% aqueous solution) was added to the resulting mixture, and the mixture was extracted with ethyl acetate. The resulting organic layer was dried over anhydrous sodium sulfate and concentrated under reduced pressure to provide 90 mg of Compound of the present invention 163 represented by the following formula.

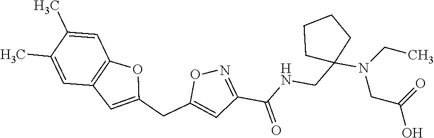 [SMILES: CCN(CC(=O)O)C1(CNC(=O)c2cc(Cc3cc4cc(C)c(C)cc4o3)on2)CCCC1]

Compound of the present invention 163: ¹H-NMR (CDCl₃) δ:7.49-7.30 (2H, m), 7.23 (1H, s), 7.18 (1H, s), 6.57 (1H, s), 6.51 (1H, s), 4.15 (2H, s), 3.60 (2H, s), 3.33 (2H, s), 2.80 (2H, q), 2.33 (3H, s), 2.30 (3H, s), 2.08-1.53 (8H, m), 1.22 (3H, t).

### Production Example 12

10 mL of methylamine (40% methanol solution) was added to 910 mg of Intermediate 74 and the mixture was stirred at room temperature for 19 hours. The resulting mixture was concentrated under reduced pressure. The resulting residue was subjected to silica gel chromatography (chloroform:methanol=5:1) to provide 420 mg of Compound of the present invention 164 represented by the following formula.

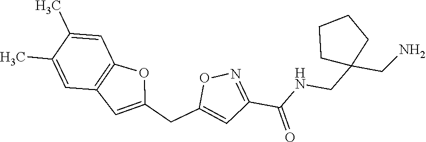 [SMILES: Cc1cc2cc(Cc3cc(C(=O)NCC4(CN)CCCC4)no3)oc2cc1C]

Compound of the present invention 164: ¹H-NMR (CDCl₃) δ: 8.66-8.54 (1H, m), 7.26 (1H, s), 7.21 (1H, s), 6.57 (1H, s), 6.46 (1H, s), 4.27 (2H, s), 3.43 (2H, d), 2.73 (2H, s), 2.34 (3H, s), 2.31 (3H, s), 1.75-1.28 (10H, m).

Then, examples of the Compound W of the present invention produced in accordance with any one of the production examples described in Examples and the production methods described in the present specification are shown below.

A compound represented by formula (L-1) (hereinafter, referred to as a compound (L-1)),

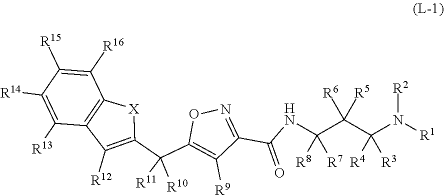

wherein R¹² is a hydrogen atom, R¹³ is a hydrogen atom, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX1).

TABLE L1

| R¹ | R² | R³ | R⁴ | R⁵ | R⁶ | R⁷ | R⁸ | R⁹ | R¹⁰ | R¹¹ |
|---|---|---|---|---|---|---|---|---|---|---|
| H | H | H | H | H | H | H | H | H | H | H |
| Me | H | H | H | H | H | H | H | H | H | H |
| Me | Me | H | H | H | H | H | H | H | H | H |
| Me | Me | Me | H | H | H | H | H | H | H | H |
| Me | Me | H | H | Me | H | H | H | H | H | H |
| Me | Me | H | H | H | H | Me | H | H | H | H |
| Me | Me | Me | Me | H | H | H | H | H | H | H |
| Me | Me | H | H | Me | Me | H | H | H | H | H |
| Me | Me | H | H | H | H | Me | Me | H | H | H |
| Me | Me | Me | Me | H | H | H | H | H | Me | H |
| Me | Me | H | H | Me | Me | H | H | H | Me | H |
| Me | Me | H | H | H | H | Me | Me | H | Me | H |
| Me | Me | Me | Me | H | H | H | H | H | OH | H |
| Me | Me | H | H | Me | Me | H | H | H | OH | H |
| Me | Me | H | H | H | H | Me | Me | H | OH | H |
| Me | Me | Me | Me | H | H | H | H | H | F | H |
| Me | Me | H | H | Me | Me | H | H | H | F | H |
| Me | Me | H | H | H | H | Me | Me | H | F | H |
| Me | Me | Me | Me | H | H | H | H | Me | H | H |
| Me | Me | H | H | Me | Me | H | H | Me | H | H |
| Me | Me | H | H | H | H | Me | Me | Me | H | H |
| Me | Me | Me | Me | H | H | H | H | F | H | H |
| Me | Me | H | H | Me | Me | H | H | F | H | H |
| Me | Me | H | H | H | H | Me | Me | F | H | H |
| Me | Me | Me | Me | H | H | H | H | C1 | H | H |
| Me | Me | H | H | Me | Me | H | H | C1 | H | H |
| Me | Me | H | H | H | H | Me | Me | Cl | H | H |
| Me | Me | Me | Me | H | H | H | H | Br | H | H |
| Me | Me | H | H | Me | Me | H | H | Br | H | H |
| Me | Me | H | H | H | H | Me | Me | Br | H | H |
| Me | Me | Me | Me | H | H | H | H | I | H | H |
| Me | Me | H | H | Me | Me | H | H | I | H | H |
| Me | Me | H | H | H | H | Me | Me | I | H | H |
| Me | Me | Me | Me | Me | Me | H | H | H | H | H |
| Me | Me | H | H | Me | Me | Me | Me | H | H | H |
| Me | Me | Me | Me | Me | Me | Me | Me | H | H | H |
| Me | Me | Me | Me | Me | Me | Me | Me | Me | H | H |
| Me | Me | Me | Me | Me | Me | Me | Me | Me | Me | H |
| Me | Me | Me | Me | Me | Me | Me | Me | Me | Me | Me |

TABLE L2

| R¹ | R² | R³ | R⁴ | R⁵ | R⁶ | R⁷ | R⁸ | R⁹ | R¹⁰ | R¹¹ |
|---|---|---|---|---|---|---|---|---|---|---|
| H | H | Me | H | H | H | H | H | H | H | H |
| H | H | H | H | Me | H | H | H | H | H | H |
| H | H | H | H | H | H | Me | H | H | H | H |
| H | H | Me | Me | H | H | H | H | H | H | H |
| H | H | H | H | Me | Me | H | H | H | H | H |
| H | H | H | H | H | H | Me | Me | H | H | H |
| H | H | Me | Me | H | H | H | H | Me | H | H |
| H | H | H | H | Me | Me | H | H | Me | H | H |
| H | H | H | H | H | H | Me | Me | Me | H | H |
| H | H | Me | Me | H | H | H | H | H | Me | H |
| H | H | H | H | Me | Me | H | H | H | Me | H |
| H | H | H | H | H | H | Me | Me | H | Me | H |
| H | H | Me | Me | Me | Me | H | H | H | H | H |
| H | H | H | H | Me | Me | Me | Me | H | H | H |
| H | H | Me | Me | Me | Me | Me | Me | H | H | H |
| H | H | Me | Me | Me | Me | Me | Me | Me | H | H |
| H | H | Me | Me | Me | Me | Me | Me | Me | Me | H |
| H | H | Me | Me | Me | Me | Me | Me | Me | Me | Me |

TABLE L3

| R¹ | R² | R³ | R⁴ | R⁵ | R⁶ | R⁷ | R⁸ | R⁹ | R¹⁰ | R¹¹ |
|---|---|---|---|---|---|---|---|---|---|---|
| Et | H | H | H | H | H | H | H | H | H | H |
| Et | Me | H | H | H | H | H | H | H | H | H |
| Et | Me | Me | H | H | H | H | H | H | H | H |
| Et | Me | H | H | Me | H | H | H | H | H | H |
| Et | Me | H | H | H | H | Me | H | H | H | H |
| Et | Me | Me | Me | H | H | H | H | H | H | H |
| Et | Me | H | H | Me | Me | H | H | H | H | H |
| Et | Me | H | H | H | H | Me | Me | H | H | H |
| Et | Me | Me | Me | H | H | H | H | Me | H | H |
| Et | Me | H | H | Me | Me | H | H | Me | H | H |
| Et | Me | H | H | H | H | Me | Me | Me | H | H |
| Et | Me | Me | Me | Me | Me | H | H | H | H | H |
| Et | Me | H | H | Me | Me | Me | Me | H | H | H |
| Et | Me | Me | Me | Me | Me | Me | Me | H | H | H |
| Et | Me | Me | Me | Me | Me | H | H | Me | H | H |
| Et | Me | H | H | Me | Me | Me | Me | Me | Me | H |
| Et | Me | Me | Me | Me | Me | Me | Me | Me | Me | Me |
| Et | Et | H | H | H | H | H | H | H | H | H |
| Et | Et | Me | H | H | H | H | H | H | H | H |
| Et | Et | H | H | Me | H | H | H | H | H | H |
| Et | Et | H | H | H | H | Me | H | H | H | H |
| Et | Et | Me | Me | H | H | H | H | H | H | H |
| Et | Et | H | H | Me | Me | H | H | H | H | H |
| Et | Et | H | H | H | H | Me | Me | H | H | H |
| Et | Et | Me | Me | H | H | H | H | Me | H | H |
| Et | Et | H | H | Me | Me | H | H | Me | H | H |
| Et | Et | H | H | H | H | Me | Me | Me | H | H |
| Et | Et | Me | Me | Me | Me | H | H | H | H | H |
| Et | Et | H | H | Me | Me | Me | Me | H | H | H |
| Et | Et | Me | Me | Me | Me | Me | Me | H | H | H |
| Et | Et | Me | Me | Me | Me | Me | Me | Me | H | H |
| Et | Et | Me | Me | Me | Me | Me | Me | Me | Me | H |
| Et | Et | Me | Me | Me | Me | Me | Me | Me | Me | Me |

TABLE L4

| R¹ | R² | R³ | R⁴ | R⁵ | R⁶ | R⁷ | R⁸ | R⁹ | R¹⁰ | R¹¹ |
|---|---|---|---|---|---|---|---|---|---|---|
| i-Pr | H | H | H | H | H | H | H | H | H | H |
| i-Pr | Me | H | H | H | H | H | H | H | H | H |
| i-Pr | Me | Me | H | H | H | H | H | H | H | H |
| i-Pr | Me | H | H | Me | H | H | H | H | H | H |
| i-Pr | Me | H | H | H | H | Me | H | H | H | H |
| i-Pr | Me | Me | Me | H | H | H | H | H | H | H |
| i-Pr | Me | H | H | Me | Me | H | H | H | H | H |
| i-Pr | Me | H | H | H | H | Me | Me | H | H | H |
| i-Pr | Me | Me | Me | H | H | H | H | Me | H | H |
| i-Pr | Me | H | H | Me | Me | H | H | Me | H | H |
| i-Pr | Me | H | H | H | H | Me | Me | Me | H | H |
| i-Pr | Me | Me | Me | Me | Me | H | H | H | H | H |
| i-Pr | Me | H | H | Me | Me | Me | Me | H | H | H |
| i-Pr | Me | Me | Me | Me | Me | Me | Me | H | H | H |
| i-Pr | Me | Me | Me | Me | Me | H | H | Me | H | H |
| i-Pr | Me | H | H | Me | Me | Me | Me | Me | Me | H |
| i-Pr | M? | Me | Me | Me | Me | Me | Me | Me | Me | Me |
| i-Pr | Et | H | H | H | H | H | H | H | H | H |
| i-Pr | Et | Me | H | H | H | H | H | H | H | H |
| i-Pr | Et | H | H | Me | H | H | H | H | H | H |
| i-Pr | Et | H | H | H | H | Me | H | H | H | H |
| i-Pr | Et | Me | Me | H | H | H | H | H | H | H |
| i-Pr | Et | H | H | Me | Me | H | H | H | H | H |
| i-Pr | Et | H | H | H | H | Me | Me | H | H | H |
| i-Pr | Et | Me | Me | H | H | H | H | Me | H | H |
| i-Pr | Et | H | H | Me | Me | H | H | Me | H | H |
| i-Pr | Et | H | H | H | H | Me | Me | Me | H | H |
| i-Pr | Et | Me | Me | Me | Me | H | H | H | H | H |
| i-Pr | Et | H | H | Me | Me | Me | Me | H | H | H |
| i-Pr | Et | Me | Me | Me | Me | Me | Me | H | H | H |
| i-Pr | Et | Me | Me | Me | Me | Me | Me | Me | H | H |
| i-Pr | Et | Me | Me | Me | Me | Me | Me | Me | Me | H |
| i-Pr | Et | Me | Me | Me | Me | Me | Me | Me | Me | Me |

TABLE L5

| R¹ | R² | R³ | R⁴ | R⁵ | R⁶ | R⁷ | R⁸ | R⁹ | R¹⁰ | R¹¹ |
|---|---|---|---|---|---|---|---|---|---|---|
| Pr | H | H | H | H | H | H | H | H | H | H |
| Pr | Me | H | H | H | H | H | H | H | H | H |
| Pr | Me | Me | H | H | H | H | H | H | H | H |
| Pr | Me | H | H | Me | H | H | H | H | H | H |
| Pr | Me | H | H | H | H | Me | H | H | H | H |
| Pr | Me | Me | Me | H | H | H | H | H | H | H |
| Pr | Me | H | H | Me | Me | H | H | H | H | H |
| Pr | Me | H | H | H | H | Me | Me | H | H | H |
| Pr | Me | Me | Me | H | H | H | H | Me | H | H |
| Pr | Me | H | H | Me | Me | H | H | Me | H | H |
| Pr | Me | H | H | H | H | Me | Me | Me | H | H |
| Pr | Me | Me | Me | Me | Me | H | H | H | H | H |
| Pr | Me | H | H | Me | Me | Me | Me | H | H | H |
| Px | Me | Me | Me | Me | Me | Me | Me | H | H | H |
| Pr | Me | Me | Me | Me | Me | H | H | Me | H | H |
| Pr | Me | H | H | Me | Me | Me | Me | Me | Me | H |
| Pr | Me | Me | Me | Me | Me | Me | Me | Me | Me | Me |

TABLE L6

| R¹ | R² | R³ | R⁴ | R⁵ | R⁶ | R⁷ | R⁸ | R⁹ | R¹⁰ | R¹¹ |
|---|---|---|---|---|---|---|---|---|---|---|
| Pr | Et | H | H | H | H | H | H | H | H | H |
| Pr | Et | Me | H | H | H | H | H | H | H | H |
| Pr | Et | H | H | Me | H | H | H | H | H | H |
| Pr | Et | H | H | H | H | Me | H | H | H | H |
| Pr | Et | Me | Me | H | H | H | H | H | H | H |
| Pr | Et | H | H | Me | Me | H | H | H | H | H |
| Pr | Et | H | H | H | H | Me | Me | H | H | H |
| Pr | Et | Me | Me | H | H | H | H | Me | H | H |
| Pr | Et | H | H | Me | Me | H | H | Me | H | H |
| Pr | Et | H | H | H | H | Me | Me | Me | H | H |
| Pr | Et | Me | Me | Me | Me | H | H | H | H | H |
| Pr | Et | H | H | Me | Me | Me | Me | H | H | H |
| Pr | Et | Me | Me | Me | Me | Me | Me | H | H | H |
| Pr | Et | Me | Me | Me | Me | Me | Me | Me | H | H |
| Pr | Et | Me | Me | Me | Me | Me | Me | Me | Me | H |
| Pr | Et | Me | Me | Me | Me | Me | Me | Me | Me | Me |
| Pr | Pr | H | H | H | H | H | H | H | H | H |
| Pr | Pr | Me | H | H | H | H | H | H | H | H |
| Pr | Pr | H | H | Me | H | H | H | H | H | H |
| Pr | Pr | H | H | H | H | Me | H | H | H | H |
| Pr | Pr | Me | Me | H | H | H | H | H | H | H |
| Pr | Pr | H | H | Me | Me | H | H | H | H | H |
| Pr | Pr | H | H | H | H | Me | Me | H | H | H |
| Pr | Pr | Me | Me | H | H | H | H | Me | H | H |
| Pr | Pr | H | H | Me | Me | H | H | Me | H | H |
| Pr | Pr | H | H | H | H | Me | Me | Me | H | H |
| Pr | Pr | Me | Me | Me | Me | H | H | H | H | H |
| Pr | Pr | H | H | Me | Me | Me | Me | H | H | H |
| Pr | Pr | Me | Me | Me | Me | Me | Me | H | H | H |
| Pr | Pr | Me | Me | Me | Me | H | H | Me | H | H |
| Pr | Pr | H | H | Me | Me | Me | Me | Me | Me | H |
| Pr | Pr | Me | Me | Me | Me | Me | Me | Me | Me | Me |

TABLE L7

| R¹ | R² | R³ | R⁴ | R⁵ | R⁶ | R⁷ | R⁸ | R⁹ | R¹⁰ | R¹¹ |
|---|---|---|---|---|---|---|---|---|---|---|
| Pr | i-Pr | H | H | H | H | H | H | H | H | H |
| Pr | i-Pr | Me | H | H | H | H | H | H | H | H |
| Pr | i-Pr | H | H | Me | H | H | H | H | H | H |
| Pr | i-Pr | H | H | H | H | Me | H | H | H | H |
| Pr | i-Pr | Me | Me | H | H | H | H | H | H | H |
| Pr | i-Pr | H | H | Me | Me | H | H | H | H | H |
| Pr | i-Pr | H | H | H | H | Me | Me | H | H | H |
| Pr | i-Pr | Me | Me | H | H | H | H | Me | H | H |
| Pr | i-Pr | H | H | Me | Me | H | H | Me | H | H |
| Pr | i-Pr | H | H | H | H | Me | Me | Me | H | H |
| Pr | i-Pr | Me | Me | Me | Me | H | H | H | H | H |
| Pr | i-Pr | H | H | Me | Me | Me | Me | H | H | H |
| Pr | i-Pr | Me | Me | Me | Me | Me | Me | H | H | H |
| Pr | i-Pr | Me | Me | Me | Me | Me | Me | Me | H | H |
| Pr | i-Pr | Me | Me | Me | Me | Me | Me | Me | Me | H |
| Pr | i-Pr | Me | Me | Me | Me | Me | Me | Me | Me | Me |
| Bu | H | H | H | H | H | H | H | H | H | H |
| Bu | Me | H | H | H | H | H | H | H | H | H |
| Bu | Me | Me | H | H | H | H | H | H | H | H |
| Bu | Me | H | H | Me | H | H | H | H | H | H |
| Bu | Me | H | H | H | H | Me | H | H | H | H |
| Bu | Me | Me | Me | H | H | H | H | H | H | H |
| Bu | Me | H | H | Me | Me | H | H | H | H | H |
| Bu | Me | H | H | H | H | Me | Me | H | H | H |
| Bu | Me | Me | Me | H | H | H | H | Me | H | H |
| Bu | Me | H | H | Me | Me | H | H | Me | H | H |
| Bu | Me | H | H | H | H | Me | Me | Me | H | H |
| Bu | Me | Me | Me | Me | Me | H | H | H | H | H |
| Bu | Me | H | H | Me | Me | Me | Me | H | H | H |
| Bu | Me | Me | Me | Me | Me | Me | Me | H | H | H |
| Bu | Me | Me | Me | Me | Me | H | H | Me | H | H |
| Bu | Me | H | H | Me | Me | Me | Me | Me | Me | H |
| Bu | Me | Me | Me | Me | Me | Me | Me | Me | Me | Me |

TABLE L8

| R¹ | R² | R³ | R⁴ | R⁵ | R⁶ | R⁷ | R⁸ | R⁹ | R¹⁰ | R¹¹ |
|---|---|---|---|---|---|---|---|---|---|---|
| Bu | Et | H | H | H | H | H | H | H | H | H |
| Bu | Et | Me | H | H | H | H | H | H | H | H |
| Bu | Et | H | H | Me | H | H | H | H | H | H |
| Bu | Et | H | H | H | H | Me | H | H | H | H |
| Bu | Et | Me | Me | H | H | H | H | H | H | H |
| Bu | Et | H | H | Me | Me | H | H | H | H | H |
| Bu | Et | H | H | H | H | Me | Me | H | H | H |
| Bu | Et | Me | Me | H | H | H | H | Me | H | H |
| Bu | Et | H | H | Me | Me | H | H | Me | H | H |
| Bu | Et | H | H | H | H | Me | Me | Me | H | H |
| Bu | Et | Me | Me | Me | Me | H | H | H | H | H |
| Bu | Et | H | H | Me | Me | Me | Me | H | H | H |
| Bu | Et | Me | Me | Me | Me | Me | Me | H | H | H |
| Bu | Et | Me | Me | Me | Me | Me | Me | Me | H | H |
| Bu | Et | Me | Me | Me | Me | Me | Me | Me | Me | H |
| Bu | Et | Me | Me | Me | Me | Me | Me | Me | Me | Me |
| Bu | i-Pr | H | H | H | H | H | H | H | H | H |
| Bu | i-Pr | Me | H | H | H | H | H | H | H | H |
| Bu | i-Pr | H | H | Me | H | H | H | H | H | H |
| Bu | i-Pr | H | H | H | H | Me | H | H | H | H |
| Bu | i-Pr | Me | Me | H | H | H | H | H | H | H |
| Bu | i-Pr | H | H | Me | Me | H | H | H | H | H |
| Bu | i-Pr | H | H | H | H | Me | Me | H | H | H |
| Bu | i-Pr | Me | Me | H | H | H | H | Me | H | H |
| Bu | i-Pr | H | H | Me | Me | H | H | Me | H | H |
| Bu | i-Pr | H | H | H | H | Me | Me | Me | H | H |
| Bu | i-Pr | Me | Me | Me | Me | H | H | H | H | H |
| Bu | i-Pr | H | H | Me | Me | Me | Me | H | H | H |
| Bu | i-Pr | Me | Me | Me | Me | Me | Me | H | H | H |
| Bu | i-Pr | Me | Me | Me | Me | H | H | Me | H | H |
| Bu | i-Pr | H | H | Me | Me | Me | Me | Me | Me | H |
| Bu | i-Pr | Me | Me | Me | Me | Me | Me | Me | Me | Me |

TABLE L9

| R¹ | R² | R³ | R⁴ | R⁵ | R⁶ | R⁷ | R⁸ | R⁹ | R¹⁰ | R¹¹ |
|---|---|---|---|---|---|---|---|---|---|---|
| Bu | Pr | H | H | H | H | H | H | H | H | H |
| Bu | Pr | Me | H | H | H | H | H | H | H | H |
| Bu | Pr | H | H | Me | H | H | H | H | H | H |
| Bu | Pr | H | H | H | H | Me | H | H | H | H |
| Bu | Pr | Me | Me | H | H | H | H | H | H | H |
| Bu | Pr | H | H | Me | Me | H | H | H | H | H |
| Bu | Pr | H | H | H | H | Me | Me | H | H | H |
| Bu | Pr | Me | Me | H | H | H | H | Me | H | H |
| Bu | Pr | H | H | Me | Me | H | H | Me | H | H |
| Bu | Pr | H | H | H | H | Me | Me | Me | H | H |
| Bu | Pr | Me | Me | Me | Me | H | H | H | H | H |
| Bu | Pr | H | H | Me | Me | Me | Me | H | H | H |
| Bu | Pr | Me | Me | Me | Me | Me | Me | H | H | H |
| Bu | Pr | Me | Me | Me | Me | Me | Me | Me | H | H |
| Bu | Pr | Me | Me | Me | Me | Me | Me | Me | Me | H |
| Bu | Pr | Me | Me | Me | Me | Me | Me | Me | Me | Me |
| Bu | Bu | H | H | H | H | H | H | H | H | H |
| Bu | Bu | Me | H | H | H | H | H | H | H | H |
| Bu | Bu | H | H | Me | H | H | H | H | H | H |
| Bu | Bu | H | H | H | H | Me | H | H | H | H |
| Bu | Bu | Me | Me | H | H | H | H | H | H | H |
| Bu | Bu | H | H | Me | Me | H | H | H | H | H |
| Bu | Bu | H | H | H | H | Me | Me | H | H | H |
| Bu | Bu | Me | Me | H | H | H | H | Me | H | H |
| Bu | Bu | H | H | Me | Me | H | H | Me | H | H |
| Bu | Bu | H | H | H | H | Me | Me | Me | H | H |
| Bu | Bu | Me | Me | Me | Me | H | H | H | H | H |
| Bu | Bu | H | H | Me | Me | Me | Me | H | H | H |
| Bu | Bu | Me | Me | Me | Me | Me | Me | H | H | H |
| Bu | Bu | Me | Me | Me | Me | H | H | Me | H | H |
| Bu | Bu | H | H | Me | Me | Me | Me | Me | Me | H |
| Bu | Bu | Me | Me | Me | Me | Me | Me | Me | Me | Me |

TABLE L10

| R¹ | R² | R³ | R⁴ | R⁵ | R⁶ | R⁷ | R⁸ | R⁹ | R¹⁰ | R¹¹ |
|---|---|---|---|---|---|---|---|---|---|---|
| t-Bu | H | H | H | H | H | H | H | H | H | H |
| t-Bu | Me | H | H | H | H | H | H | H | H | H |
| t-Bu | Me | Me | H | H | H | H | H | H | H | H |
| t-Bu | Me | H | H | Me | H | H | H | H | H | H |
| t-Bu | Me | H | H | H | H | Me | H | H | H | H |
| t-Bu | Me | Me | Me | H | H | H | H | H | H | H |
| t-Bu | Me | H | H | Me | Me | H | H | H | H | H |
| t-Bu | Me | H | H | H | H | Me | Me | H | H | H |
| t-Bu | Me | Me | Me | H | H | H | H | Me | H | H |
| t-Bu | Me | H | H | Me | Me | H | H | Me | H | H |
| t-Bu | Me | H | H | H | H | Me | Me | Me | H | H |
| t-Bu | Me | Me | Me | Me | Me | H | H | H | H | H |
| t-Bu | Me | H | H | Me | Me | Me | Me | H | H | H |
| t-Bu | Me | Me | Me | Me | Me | Me | Me | H | H | H |
| t-Bu | Me | Me | Me | Me | Me | H | H | M? | H | H |
| t-Bu | Me | H | H | Me | Me | Me | Me | Me | Me | H |
| t-Bu | Me | Me | Me | Me | Me | Me | Me | Me | Me | Me |
| t-Bu | Et | H | H | H | H | H | H | H | H | H |
| t-Bu | Et | Me | H | H | H | H | H | H | H | H |
| t-Bu | Et | H | H | Me | H | H | H | H | H | H |
| t-Bu | Et | H | H | H | H | Me | H | H | H | H |
| t-Bu | Et | Me | Me | H | H | H | H | H | H | H |
| t-Bu | Et | H | H | Me | Me | H | H | H | H | H |
| t-Bu | Et | H | H | H | H | Me | Me | H | H | H |
| t-Bu | Et | Me | Me | H | H | H | H | Me | H | H |
| t-Bu | Et | H | H | Me | Me | H | H | Me | H | H |
| t-Bu | Et | H | H | H | H | Me | Me | Me | H | H |
| t-Bu | Et | Me | Me | Me | Me | H | H | H | H | H |
| t-Bu | Et | H | H | Me | Me | Me | Me | H | H | H |
| t-Bu | Et | Me | Me | Me | Me | Me | Me | H | H | H |
| t-Bu | Et | Me | Me | Me | Me | Me | Me | Me | H | H |
| t-Bu | Et | Me | Me | Me | Me | Me | Me | Me | Me | H |
| t-Bu | Et | Me | Me | Me | Me | Me | Me | Me | Me | Me |

TABLE L11

| R¹ | R² | R³ | R⁴ | R⁵ | R⁶ | R⁷ | R⁸ | R⁹ | R¹⁰ | R¹¹ |
|---|---|---|---|---|---|---|---|---|---|---|
| t-Bu | Pr | H | H | H | H | H | H | H | H | H |
| t-Bu | Pr | Me | H | H | H | H | H | H | H | H |
| t-Bu | Pr | H | H | Me | H | H | H | H | H | H |
| t-Bu | Pr | H | H | H | H | Me | H | H | H | H |
| t-Bu | Pr | Me | Me | H | H | H | H | H | H | H |
| t-Bu | Pr | H | H | Me | Me | H | H | H | H | H |
| t-Bu | Pr | H | H | H | H | Me | Me | H | H | H |
| t-Bu | Pr | Me | Me | H | H | H | H | Me | H | H |
| t-Bu | Pr | H | H | Me | Me | B | H | Me | H | H |
| t-Bu | Pr | H | H | H | H | Me | Me | Me | H | H |
| t-Bu | Pr | Me | Me | Me | Me | H | H | H | H | H |
| t-Bu | Pr | H | H | Me | Me | Me | Me | H | H | H |
| t-Bu | Pr | Me | Me | Me | Me | Me | Me | H | H | H |
| t-Bu | Pr | Me | Me | Me | Me | Me | Me | Me | H | H |
| t-Bu | Pr | Me | Me | Me | Me | Me | Me | Me | Me | H |
| t-Bu | Pr | Me | Me | Me | Me | Me | Me | Me | Me | Me |

TABLE L12

| R¹ | R² | R³ | R⁴ | R⁵ | R⁶ | R⁷ | R⁸ | R⁹ | R¹⁰ | R¹¹ |
|---|---|---|---|---|---|---|---|---|---|---|
| t-Bu | Bu | H | H | H | H | H | H | H | H | H |
| t-Bu | Bu | Me | H | H | H | H | H | H | H | H |
| t-Bu | Bu | H | H | Me | H | H | H | H | H | H |
| t-Bu | Bu | H | H | H | HI | Me | H | H | H | H |
| t-Bu | Bu | Me | Me | H | H | H | H | H | H | H |
| t-Bu | Bu | H | H | Me | Me | H | H | H | H | H |
| t-Bu | Bu | H | H | H | H | Me | Me | H | H | H |
| t-Bu | Bu | Me | Me | H | H | H | H | Me | H | H |
| t-Bu | Bu | H | H | Me | Me | H | H | Me | H | H |
| t-Bu | Bu | H | H | H | H | Me | Me | Me | H | H |
| t-Bu | Bu | Me | Me | Me | Me | H | H | H | H | H |
| t-Bu | Bu | H | H | Me | Me | Me | Me | H | H | H |
| t-Bu | Bu | Me | Me | Me | Me | Me | Me | H | H | H |
| t-Bu | Bu | Me | Me | Me | Me | H | H | Me | H | H |
| t-Bu | Bu | H | H | Me | Me | Me | Me | Me | Me | H |
| t-Bu | Bu | Me | Me | Me | Me | Me | Me | Me | Me | Me |
| Me | H | Me | Me | H | H | H | H | H | H | H |
| Me | H | H | H | Me | Me | H | H | H | H | H |
| Me | H | H | H | H | H | Me | Me | H | H | H |
| Me | H | Me | Me | Me | Me | H | H | H | H | H |
| Me | H | H | H | Me | Me | Me | Me | H | H | H |
| Me | H | Me | Me | Me | Me | Me | Me | H | H | H |
| Me | H | Me | Me | Me | Me | H | H | Me | H | H |
| Me | H | H | H | Me | Me | Me | Me | Me | Me | H |
| Me | H | Me | Me | Me | Me | Me | Me | Me | Me | Me |
| Et | H | Me | Me | H | H | H | H | H | H | H |
| Et | H | H | H | Me | Me | H | H | H | H | H |
| Et | H | H | H | H | H | Me | Me | H | H | H |
| Et | H | Me | Me | Me | Me | H | H | H | H | H |
| Et | H | H | H | Me | Me | Me | Me | H | H | H |
| Et | H | Me | Me | Me | Me | Me | Me | H | H | H |
| Et | H | Me | Me | Me | Me | H | H | Me | H | H |
| Et | H | H | H | Me | Me | Me | Me | Me | Me | H |
| Et | H | Me | Me | Me | Me | Me | Me | Me | Me | Me |

TABLE L13

| R¹ | R² | R³ | R⁴ | R⁵ | R⁶ | R⁷ | R⁸ | R⁹ | R¹⁰ | R¹¹ |
|---|---|---|---|---|---|---|---|---|---|---|
| i-Pr | H | Me | Me | H | H | H | H | H | H | H |
| i-Pr | H | H | H | Me | Me | H | H | H | H | H |
| i-Pr | H | H | H | H | H | Me | Me | H | H | H |
| i-Pr | H | Me | Me | Me | Me | H | H | H | H | H |
| i-Pr | H | H | H | Me | Me | Me | Me | H | H | H |
| i-Pr | H | Me | Me | Me | Me | Me | Me | H | H | H |
| i-Pr | H | Me | Me | Me | Me | H | H | Me | H | H |
| i-Pr | H | H | H | Me | Me | Me | Me | Me | Me | H |
| i-Pr | H | Me | Me | Me | Me | Me | Me | Me | Me | Me |
| Pr | H | Me | Me | H | H | H | H | H | H | H |
| Pr | H | H | H | Me | Me | H | H | H | H | H |
| Pr | H | H | H | H | H | Me | Me | H | H | H |
| Pr | H | Me | Me | Me | Me | H | H | H | H | H |
| Pr | H | H | H | Me | Me | Me | Me | H | H | H |
| Pr | H | Me | Me | Me | Me | Me | Me | H | H | H |
| Pr | H | Me | Me | Me | Me | H | H | Me | H | H |
| Pr | H | H | H | Me | Me | Me | Me | Me | Me | H |
| Pr | H | Me | Me | Me | Me | Me | Me | Me | Me | Me |
| Bu | H | Me | Me | H | H | H | H | H | H | H |
| Bu | H | H | H | Me | Me | H | H | H | H | H |
| Bu | H | H | H | H | H | Me | Me | H | H | H |
| Bu | H | Me | Me | Me | Me | H | H | H | H | H |
| Bu | H | H | H | Me | Me | Me | Me | H | H | H |
| Bu | H | Me | Me | Me | Me | Me | Me | H | H | H |
| Bu | H | Me | Me | Me | Me | H | H | Me | H | H |
| Bu | H | H | H | Me | Me | Me | Me | Me | Me | H |
| Bu | H | Me | Me | Me | Me | Me | Me | Me | Me | Me |
| t-Bu | H | Me | Me | H | H | H | H | H | H | H |
| t-Bu | H | H | H | Me | Me | H | H | H | H | H |
| t-Bu | H | H | H | H | H | Me | Me | H | H | H |
| t-Bu | H | Me | Me | Me | Me | H | H | H | H | H |
| t-Bu | H | H | H | Me | Me | Me | Me | H | H | H |
| t-Bu | H | Me | Me | Me | Me | Me | Me | H | H | H |
| t-Bu | H | Me | Me | Me | Me | H | H | Me | H | H |
| t-Bu | H | H | H | Me | Me | Me | Me | Me | Me | H |
| t-Bu | H | Me | Me | Me | Me | Me | Me | Me | Me | Me |

The compound (L-1), wherein R¹² is a methyl group, R¹³ is a hydrogen atom, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX2).

The compound (L-1), wherein R¹² is a hydrogen atom, R¹³ is a methyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX3).

The compound (L-1), wherein R¹² is a hydrogen atom, R¹³ is a hydrogen atom, R¹⁴ is a methyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX4).

The compound (L-1), wherein R¹² is a hydrogen atom, R¹³ is a hydrogen atom, R¹⁴ is a hydrogen atom, R¹⁵ is a methyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX5).

The compound (L-1), wherein R¹² is a hydrogen atom, R¹³ is a hydrogen atom, R¹⁴ is a hydrogen atom, R¹⁵ is a hydrogen atom, R¹⁶ is a methyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX6).

The compound (L-1), wherein R¹² is a hydrogen atom, R¹³ is a methyl group, R¹⁴ is a methyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX7).

The compound (L-1), wherein R¹² is a hydrogen atom, R¹³ is a methyl group, R¹⁴ is a hydrogen atom, R¹⁵ is a methyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX8).

The compound (L-1), wherein R¹² is a hydrogen atom, R¹³ is a methyl group, R¹⁴ is a hydrogen atom, R¹⁵ is a hydrogen atom, R¹⁶ is a methyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX9).

The compound (L-1), wherein R¹² is a hydrogen atom, R¹³ is a hydrogen atom, R¹⁴ is a methyl group, R¹⁵ is a methyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX10).

The compound (L-1), wherein R¹² is a hydrogen atom, R¹³ is a hydrogen atom, R¹⁴ is a methyl group, R¹⁵ is a hydrogen atom, R¹⁶ is a methyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX11).

The compound (L-1), wherein R¹² is a hydrogen atom, R¹³ is a hydrogen atom, R¹⁴ is a hydrogen atom, R¹⁵ is a methyl group, R¹⁶ is a methyl group, X is an oxygen atom, and the combination of R¹, R², R³, R⁴, R⁵, R⁶, R⁷, R⁸, R⁹, group SX12).

The compound (L-1), wherein R¹² is a hydrogen atom, R¹³ is a methyl group, R¹⁴ is a methyl group, R¹⁵ is a methyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX13).

The compound (L-1), wherein R¹² is a hydrogen atom, R¹³ is a hydrogen atom, R¹⁴ is a methyl group, R¹⁵ is a methyl group, R¹⁶ is a methyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX14).

The compound (L-1), wherein R¹² is a hydrogen atom, R¹³ is a fluorine atom, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX15).

The compound (L-1), wherein R¹² is a hydrogen atom, R¹³ is a hydrogen atom, R¹⁴ is a fluorine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX16).

The compound (L-1), wherein R¹² is a hydrogen atom, R¹³ is a hydrogen atom, R¹⁴ is a hydrogen atom, R¹⁵ is a fluorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX17).

The compound (L-1), wherein R¹² is a hydrogen atom, R¹³ is a hydrogen atom, R¹⁴ is a hydrogen atom, R¹⁵ is a hydrogen atom, R¹⁶ is a fluorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX18).

The compound (L-1), wherein R¹² is a hydrogen atom, R¹³ is a chlorine atom, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX19).

The compound (L-1), wherein R¹² is a hydrogen atom, R¹³ is a hydrogen atom, R¹⁴ is a chlorine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX20).

The compound (L-1), wherein R¹² is a hydrogen atom, R¹³ is a hydrogen atom, R¹⁴ is a hydrogen atom, R¹⁵ is a chlorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX21).

The compound (L-1), wherein R¹² is a hydrogen atom, R¹³ is a hydrogen atom, R¹⁴ is a hydrogen atom, R¹⁵ is a hydrogen atom, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX22).

The compound (L-1), wherein R¹² is a hydrogen atom, R¹³ is a bromine atom, R¹⁴ is a hydrogen atom, R¹⁵ is a R¹⁰, and R¹¹ is any one of the combinations shown in [Table L1] to [Table L13] (hereinafter, referred to as a compound group SX23).

The compound (L-1), wherein R¹² is a hydrogen atom, R¹³ is a hydrogen atom, R¹⁴ is a bromine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX24).

The compound (L-1), wherein R¹² is a hydrogen atom, R¹³ is a hydrogen atom, R¹⁴ is a hydrogen atom, R¹⁵ is a bromine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX25).

The compound (L-1), wherein R¹² is a hydrogen atom, R¹³ is a hydrogen atom, R¹⁴ is a hydrogen atom, R¹⁵ is a hydrogen atom, R¹⁶ is a bromine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX26).

The compound (L-1), wherein R¹² is a hydrogen atom, R¹³ is an iodine atom, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX27).

The compound (L-1), wherein R¹² is a hydrogen atom, R¹³ is a hydrogen atom, R¹⁴ is an iodine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX28).

The compound (L-1), wherein R¹² is a hydrogen atom, R¹³ is a hydrogen atom, R¹⁴ is a hydrogen atom, R¹⁵ is an iodine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX29).

The compound (L-1), wherein R¹² is a hydrogen atom, R¹³ is a hydrogen atom, R¹⁴ is a hydrogen atom, R¹⁵ is a hydrogen atom, R¹⁶ is an iodine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX30).

The compound (L-1), wherein R¹² is a hydrogen atom, R¹³ is a methoxy group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX31).

The compound (L-1), wherein R¹² is a hydrogen atom, R¹³ is a hydrogen atom, R¹⁴ is a methoxy group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX32).

The compound (L-1), wherein R¹² is a hydrogen atom, R¹³ is a hydrogen atom, R¹⁴ is a hydrogen atom, R¹⁵ is a methoxy group, Rue is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX33).

The compound (1-1), wherein R¹² is a hydrogen atom, R¹³ is a hydrogen atom, R¹⁴ is a hydrogen atom, R¹⁵ is a hydrogen atom, R¹⁶ is a methoxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX34).

The compound (L-1), wherein R¹² is a hydrogen atom, R¹³ is a trifluoromethyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX35).

The compound (L-1), wherein R¹² is a hydrogen atom, R¹³ is a hydrogen atom, R¹⁴ is a trifluoromethyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX36).

The compound (L-1), wherein R¹² is a hydrogen atom, R¹³ is a hydrogen atom, R¹⁴ is a hydrogen atom, R¹⁵ is a trifluoromethyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX37).

The compound (L-1), wherein R¹² is a hydrogen atom, R¹³ is a hydrogen atom, R¹⁴ is a hydrogen atom, R¹⁵ is a hydrogen atom, R¹⁶ is a trifluoromethyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX38).

The compound (L-1), wherein R¹² is a hydrogen atom, R¹³ is a cyclopropyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX39).

The compound (L-1), wherein R¹² is a hydrogen atom, R¹³ is a hydrogen atom, R¹⁴ is a cyclopropyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX40).

The compound (L-1), wherein R¹² is a hydrogen atom, R¹³ is a hydrogen atom, R¹⁴ is a hydrogen atom, R¹⁵ is a cyclopropyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX41).

The compound (L-1), wherein R¹² is a hydrogen atom, R¹³ is a hydrogen atom, R¹⁴ is a hydrogen atom, R¹⁵ is a hydrogen atom, R¹⁶ is a cyclopropyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX42).

The compound (L-1), wherein R¹² is a hydrogen atom, R¹³ is a cyclobutyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX43).

The compound (L-1), wherein R¹² is a hydrogen atom, R¹³ is a hydrogen atom, R¹⁴ is a cyclobutyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX44).

The compound (L-1), wherein R¹² is a hydrogen atom, R¹³ is a hydrogen atom, R¹⁴ is a hydrogen atom, R¹⁵ is a cyclobutyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX45).

The compound (L-1), wherein R¹² is a hydrogen atom, R¹³ is a hydrogen atom, R¹⁴ is a hydrogen atom, R¹⁵ is a hydrogen atom, R¹⁶ is a cyclobutyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX46).

The compound (L-1), wherein R¹² is a hydrogen atom, R¹³ is a cyclopentyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX47).

The compound (L-1), wherein R¹² is a hydrogen atom, R¹³ is a hydrogen atom, R¹⁴ is a cyclopentyl group, R¹⁵ is a R¹⁰, and R¹¹ is any one of the combinations shown in [Table L1] to [Table L13] (hereinafter, referred to as a compound group SX48).

The compound (L-1), wherein R¹² is a hydrogen atom, R¹³ is a hydrogen atom, R¹⁴ is a hydrogen atom, R¹⁵ is a cyclopentyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX49).

The compound (L-1), wherein R¹² is a hydrogen atom, R¹³ is a hydrogen atom, R¹⁴ is a hydrogen atom, R¹⁵ is a hydrogen atom, R¹⁶ is a cyclopentyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX50).

The compound (L-1), wherein R¹² is a hydrogen atom, R¹³ is a cyclohexyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX51).

The compound (L-1), wherein R¹² is a hydrogen atom, R¹³ is a hydrogen atom, R¹⁴ is a cyclohexyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX52).

The compound (L-1), wherein R¹² is a hydrogen atom, R¹³ is a hydrogen atom, R¹⁴ is a hydrogen atom, R¹⁵ is a cyclohexyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX53).

The compound (L-1), wherein R¹² is a hydrogen atom, R¹³ is a hydrogen atom, R¹⁴ is a hydrogen atom, R¹⁵ is a hydrogen atom, R¹⁶ is a cyclohexyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX54).

The compound (L-1), wherein R¹² is a hydrogen atom, R¹³ is a phenyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX55).

The compound (L-1), wherein R¹² is a hydrogen atom, R¹³ is a hydrogen atom, R¹⁴ is a phenyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX56).

The compound (L-1), wherein R¹² is a hydrogen atom, R¹³ is a hydrogen atom, R¹⁴ is a hydrogen atom, R¹⁵ is a phenyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX57).

The compound (L-1), wherein R¹² is a hydrogen atom, R¹³ is a hydrogen atom, R¹⁴ is a hydrogen atom, R¹⁵ is a hydrogen atom, R¹⁶ is a phenyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX58).

The compound (L-1), wherein R¹² is a hydrogen atom, R¹³ is an acetyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX59).

The compound (L-1), wherein R¹² is a hydrogen atom, R¹³ is a hydrogen atom, R¹⁴ is an acetyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX60).

The compound (L-1), wherein R¹² is a hydrogen atom, R¹³ is a hydrogen atom, R¹⁴ is a hydrogen atom, R¹⁵ is an acetyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX61).

The compound (L-1), wherein R¹² is a hydrogen atom, R¹³ is a hydrogen atom, R¹⁴ is a hydrogen atom, R¹⁵ is a hydrogen atom, R¹⁶ is an acetyl group, X is an oxygen atom, and the combination of R¹, R², R³, R⁴, R⁵, R⁶, R⁷, R⁸, R⁹, group SX62).

The compound (L-1), wherein R¹² is a hydrogen atom, R¹³ is a trifluoroacetyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX63).

The compound (L-1), wherein R¹² is a hydrogen atom, R¹³ is a hydrogen atom, R¹⁴ is a trifluoroacetyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX64).

The compound (L-1), wherein R¹² is a hydrogen atom, R¹³ is a hydrogen atom, R¹⁴ is a hydrogen atom, R¹⁵ is a trifluoroacetyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX65).

The compound (L-1), wherein R¹² is a hydrogen atom, R¹³ is a hydrogen atom, R¹⁴ is a hydrogen atom, R¹⁵ is a hydrogen atom, R¹⁶ is a trifluoroacetyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX66).

The compound (1-1), wherein R¹² is a hydrogen atom, R¹³ is a methoxycarbonyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX67).

The compound (L-1), wherein R¹² is a hydrogen atom, R¹³ is a hydrogen atom, R¹⁴ is a methoxycarbonyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX68).

The compound (L-1), wherein R¹² is a hydrogen atom, R¹³ is a hydrogen atom, R¹⁴ is a hydrogen atom, R¹⁵ is a methoxycarbonyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX69).

The compound (L-1), wherein R¹² is a hydrogen atom, R¹³ is a hydrogen atom, R¹⁴ is a hydrogen atom, R¹⁵ is a hydrogen atom, R¹⁶ is a methoxycarbonyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX70).

The compound (L-1), wherein R¹² is a hydrogen atom, R¹³ is a trifluoromethoxycarbonyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX71).

The compound (L-1), wherein R¹² is a hydrogen atom, R¹³ is a hydrogen atom, R¹⁴ is a trifluoromethoxycarbonyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX72).

The compound (L-1), wherein R¹² is a hydrogen atom, R¹³ is a hydrogen atom, R¹⁴ is a hydrogen atom, R¹⁵ is a trifluoromethoxycarbonyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX73).

The compound (L-1), wherein R¹² is a hydrogen atom, R¹³ is a hydrogen atom, R¹⁴ is a hydrogen atom, R¹⁵ is a hydrogen atom, R¹⁶ is a trifluoromethoxycarbonyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX74).

The compound (L-1), wherein R¹² is a hydrogen atom, R¹³ is an acetoxy group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX75).

The compound (L-1), wherein R¹² is a hydrogen atom, R¹³ is a hydrogen atom, R¹⁴ is an acetoxy group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX76).

The compound (L-1), wherein R¹² is a hydrogen atom, R¹³ is a hydrogen atom, R¹⁴ is a hydrogen atom, R¹⁵ is an acetoxy group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX77).

The compound (L-1), wherein R¹² is a hydrogen atom, R¹³ is a hydrogen atom, R¹⁴ is a hydrogen atom, R¹⁵ is a hydrogen atom, R¹⁶ is an acetoxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX78).

The compound (L-1), wherein R¹² is a hydrogen atom, R¹³ is a trifluoroacetoxy group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX79).

The compound (L-1), wherein R¹² is a hydrogen atom, R¹³ is a hydrogen atom, R¹⁴ is a trifluoroacetoxy group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX80).

The compound (L-1), wherein R¹² is a hydrogen atom, R¹³ is a hydrogen atom, R¹⁴ is a hydrogen atom, R¹⁵ is a trifluoroacetoxy group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX81).

The compound (L-1), wherein R¹² is a hydrogen atom, R¹³ is a hydrogen atom, R¹⁴ is a hydrogen atom, R¹⁵ is a hydrogen atom, R¹⁶ is a trifluoroacetoxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX82).

The compound (L-1), wherein R¹² is a hydrogen atom, R¹³ is a cyano group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX83).

The compound (L-1), wherein R¹² is a hydrogen atom, R¹³ is a hydrogen atom, R¹⁴ is a cyano group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX84).

The compound (L-1), wherein R¹² is a hydrogen atom, R¹³ is a hydrogen atom, R¹⁴ is a hydrogen atom, R¹⁵ is a cyano group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX85).

The compound (L-1), wherein R¹² is a hydrogen atom, R¹³ is a hydrogen atom, R¹⁴ is a hydrogen atom, R¹⁵ is a hydrogen atom, R¹⁶ is a cyano group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX86).

The compound (L-1), wherein R¹² is a hydrogen atom, R¹³ is a formyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX87).

The compound (L-1), wherein R¹² is a hydrogen atom, R¹³ is a hydrogen atom, R¹⁴ is a formyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX88).

The compound (L-1), wherein R¹² is a hydrogen atom, R¹³ is a hydrogen atom, R¹⁴ is a hydrogen atom, R¹⁵ is a formyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX89).

The compound (L-1), wherein R¹² is a hydrogen atom, R¹³ is a hydrogen atom, R¹⁴ is a hydrogen atom, R¹⁵ is a hydrogen atom, R¹⁶ is a formyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX90).

The compound (L-1), wherein R¹² is a hydrogen atom, R¹³ is a carboxy group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX91).

The compound (L-1), wherein R¹² is a hydrogen atom, R¹³ is a hydrogen atom, R¹⁴ is a carboxy group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX92).

The compound (L-1), wherein R¹² is a hydrogen atom, R¹³ is a hydrogen atom, R¹⁴ is a hydrogen atom, R¹⁵ is a carboxy group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX93).

The compound (L-1), wherein R¹² is a hydrogen atom, R¹³ is a hydrogen atom, R¹⁴ is a hydrogen atom, R¹⁵ is a hydrogen atom, R¹⁶ is a carboxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX94).

The compound (L-1), wherein R¹² is a hydrogen atom, R¹³ is a nitro group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX95).

The compound (L-1), wherein R¹² is a hydrogen atom, R¹³ is a hydrogen atom, R¹⁴ is a nitro group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX96).

The compound (L-1), wherein R¹² is a hydrogen atom, R¹³ is a hydrogen atom, R¹⁴ is a hydrogen atom, R¹⁵ is a nitro group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX97).

The compound (L-1), wherein R¹² is a hydrogen atom, R¹³ is a hydrogen atom, R¹⁴ is a hydrogen atom, R¹⁵ is a hydrogen atom, R¹⁶ is a nitro group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX98).

The compound (L-1), wherein R¹² is a hydrogen atom, R¹³ is a hydroxy group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX99).

The compound (L-1), wherein R¹² is a hydrogen atom, R¹³ is a hydrogen atom, R¹⁴ is a hydroxy group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX100).

The compound (L-1), wherein R¹² is a hydrogen atom, R¹³ is a hydrogen atom, R¹⁴ is a hydrogen atom, R¹⁵ is a hydroxy group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX101).

The compound (L-1), wherein R¹² is a hydrogen atom, R¹³ is a hydrogen atom, R¹⁴ is a hydrogen atom, R¹⁵ is a hydrogen atom, R¹⁶ is a hydroxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX102).

The compound (L-1), wherein R¹² is a hydrogen atom, R¹³ is a fluorine atom, R¹⁴ is a fluorine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX103).

The compound (L-1), wherein R¹² is a hydrogen atom, R¹³ is a fluorine atom, R¹⁴ is a hydrogen atom, R¹⁵ is a fluorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX104).

The compound (L-1), wherein R¹² is a hydrogen atom, R¹³ is a fluorine atom, R¹⁴ is a hydrogen atom, R¹⁵ is a hydrogen atom, R¹⁶ is a fluorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX105).

The compound (L-1), wherein R¹² is a hydrogen atom, R¹³ is a hydrogen atom, R¹⁴ is a fluorine atom, R¹⁵ is a fluorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX106).

The compound (L-1), wherein R¹² is a hydrogen atom, R¹³ is a hydrogen atom, R¹⁴ is a fluorine atom, R¹⁵ is a hydrogen atom, R¹⁶ is a fluorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX107).

The compound (L-1), wherein R¹² is a hydrogen atom, R¹³ is a hydrogen atom, R¹⁴ is a hydrogen atom, R¹⁵ is a fluorine atom, R¹⁶ is a fluorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX108).

The compound (L-1), wherein R¹² is a hydrogen atom, R¹³ is a fluorine atom, R¹⁴ is a fluorine atom, R¹⁵ is a fluorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX109).

The compound (1-1), wherein R¹² is a hydrogen atom, R¹³ is a hydrogen atom, R¹⁴ is a fluorine atom, R¹⁵ is a fluorine atom, R¹⁶ is a fluorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX110).

The compound (L-1), wherein R¹² is a hydrogen atom, R¹³ is a chlorine atom, R¹⁴ is a chlorine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX111).

The compound (L-1), wherein R¹² is a hydrogen atom, R¹³ is a chlorine atom, R¹⁴ is a hydrogen atom, R¹⁵ is a chlorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX112).

The compound (L-1), wherein R¹² is a hydrogen atom, R¹³ is a chlorine atom, R¹⁴ is a hydrogen atom, R¹⁵ is a hydrogen atom, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX113).

The compound (L-1), wherein R¹² is a hydrogen atom, R¹³ is a hydrogen atom, R¹⁴ is a chlorine atom, R¹⁵ is a chlorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX114).

The compound (L-1), wherein R¹² is a hydrogen atom, R¹³ is a hydrogen atom, R¹⁴ is a chlorine atom, R¹⁵ is a hydrogen atom, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX115).

The compound (L-1), wherein R¹² is a hydrogen atom, R¹³ is a hydrogen atom, R¹⁴ is a hydrogen atom, R¹⁵ is a chlorine atom, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX116).

The compound (L-1), wherein R¹² is a hydrogen atom, R¹³ is a chlorine atom, R¹⁴ is a chlorine atom, R¹⁵ is a chlorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX117).

The compound (L-1), wherein R¹² is a hydrogen atom, R¹³ is a hydrogen atom, R¹⁴ is a chlorine atom, R¹⁵ is a chlorine atom, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX118).

The compound (L-1), wherein R¹² is a hydrogen atom, R¹³ is a bromine atom, R¹⁴ is a bromine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX119).

The compound (L-1), wherein R¹² is a hydrogen atom, R¹³ is a bromine atom, R¹⁴ is a hydrogen atom, R¹⁵ is a bromine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX120).

The compound (L-1), wherein R¹² is a hydrogen atom, R¹³ is a bromine atom, R¹⁴ is a hydrogen atom, R¹⁵ is a hydrogen atom, R¹⁶ is a bromine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX121).

The compound (L-1), wherein R¹² is a hydrogen atom, R¹³ is a hydrogen atom, R¹⁴ is a bromine atom, R¹⁵ is a bromine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX122).

The compound (L-1), wherein R¹² is a hydrogen atom, R¹³ is a hydrogen atom, R¹⁴ is a bromine atom, R¹⁵ is a hydrogen atom, R¹⁶ is a bromine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX123).

The compound (L-1), wherein R¹² is a hydrogen atom, R¹³ is a hydrogen atom, R¹⁴ is a hydrogen atom, R¹⁵ is a bromine atom, R¹⁶ is a bromine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX124).

The compound (L-1), wherein R¹² is a hydrogen atom, R¹³ is a bromine atom, R¹⁴ is a bromine atom, R¹⁵ is a bromine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX125).

The compound (L-1), wherein R¹² is a hydrogen atom, R¹³ is a hydrogen atom, R¹⁴ is a bromine atom, R¹⁵ is a bromine atom, R¹⁶ is a bromine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX126).

The compound (L-1), wherein R¹² is a hydrogen atom, R¹³ is an iodine atom, R¹⁴ is an iodine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX127).

The compound (L-1), wherein R¹² is a hydrogen atom, R¹³ is an iodine atom, R¹⁴ is a hydrogen atom, R¹⁵ is an iodine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX128).

The compound (L-1), wherein R¹² is a hydrogen atom, R¹³ is an iodine atom, R¹⁴ is a hydrogen atom, R¹⁵ is a hydrogen atom, R¹⁶ is an iodine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX129).

The compound (L-1), wherein R¹² is a hydrogen atom, R¹³ is a hydrogen atom, R¹⁴ is an iodine atom, R¹⁵ is an iodine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX130).

The compound (L-1), wherein R¹² is a hydrogen atom, R¹³ is a hydrogen atom, R¹⁴ is an iodine atom, R¹⁵ is a hydrogen atom, R¹⁶ is an iodine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX131).

The compound (L-1), wherein R¹² is a hydrogen atom, R¹³ is a hydrogen atom, R¹⁴ is a hydrogen atom, R¹⁵ is an iodine atom, R¹⁶ is an iodine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX132).

The compound (L-1), wherein R¹² is a hydrogen atom, R¹³ is an iodine atom, R¹⁴ is an iodine atom, R¹⁵ is an iodine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX133).

The compound (L-1), wherein R¹² is a hydrogen atom, R¹³ is a hydrogen atom, R¹⁴ is an iodine atom, R¹⁵ is an iodine atom, R¹⁶ is an iodine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX134).

The compound (L-1), wherein R¹² is a hydrogen atom, R¹³ is a fluorine atom, R¹⁴ is a chlorine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX135).

The compound (L-1), wherein R¹² is a hydrogen atom, R¹³ is a fluorine atom, R¹⁴ is a hydrogen atom, R¹⁵ is a chlorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX136).

The compound (L-1), wherein R¹² is a hydrogen atom, R¹³ is a fluorine atom, R¹⁴ is a hydrogen atom, R¹⁵ is a hydrogen atom, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX137).

The compound (L-1), wherein R¹² is a hydrogen atom, R¹³ is a chlorine atom, R¹⁴ is a fluorine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX138).

The compound (L-1), wherein R¹² is a hydrogen atom, R¹³ is a chlorine atom, R¹⁴ is a hydrogen atom, R¹⁵ is a fluorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX139).

The compound (L-1), wherein R¹² is a hydrogen atom, R¹³ is a chlorine atom, R¹⁴ is a hydrogen atom, R¹⁵ is a hydrogen atom, R¹⁶ is a fluorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX140).

The compound (L-1), wherein R¹² is a hydrogen atom, R¹³ is a hydrogen atom, R¹⁴ is a fluorine atom, R¹⁵ is a chlorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX141).

The compound (1-1), wherein R¹² is a hydrogen atom, R¹³ is a hydrogen atom, R¹⁴ is a fluorine atom, R¹⁵ is a hydrogen atom, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX142).

The compound (L-1), wherein R¹² is a hydrogen atom, R¹³ is a hydrogen atom, R¹⁴ is a chlorine atom, R¹⁵ is a fluorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX143).

The compound (L-1), wherein R¹² is a hydrogen atom, R¹³ is a hydrogen atom, R¹⁴ is a chlorine atom, R¹⁵ is a hydrogen atom, R¹⁶ is a fluorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX144).

The compound (L-1), wherein R¹² is a hydrogen atom, R¹³ is a hydrogen atom, R¹⁴ is a hydrogen atom, R¹⁵ is a fluorine atom, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX145).

The compound (L-1), wherein R¹² is a hydrogen atom, R¹³ is a hydrogen atom, R¹⁴ is a hydrogen atom, R¹⁵ is a chlorine atom, R¹⁶ is a fluorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX146).

The compound (L-1), wherein R¹² is a hydrogen atom, R¹³ is a fluorine atom, R¹⁴ is a methyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX147).

The compound (L-1), wherein R¹² is a hydrogen atom, R¹³ is a fluorine atom, R¹⁴ is a hydrogen atom, R¹⁵ is a methyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX148).

The compound (L-1), wherein R¹² is a hydrogen atom, R¹³ is a fluorine atom, R¹⁴ is a hydrogen atom, R¹⁵ is a hydrogen atom, R¹⁶ is a methyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX149).

The compound (L-1), wherein R¹² is a hydrogen atom, R¹³ is a methyl group, R¹⁴ is a fluorine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX150).

The compound (L-1), wherein R¹² is a hydrogen atom, R¹³ is a methyl group, R¹⁴ is a hydrogen atom, R¹⁵ is a fluorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX151).

The compound (L-1), wherein R¹² is a hydrogen atom, R¹³ is a methyl group, R¹⁴ is a hydrogen atom, R¹⁵ is a hydrogen atom, R¹⁶ is a fluorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX152).

The compound (L-1), wherein R¹² is a hydrogen atom, R¹³ is a hydrogen atom, R¹⁴ is a fluorine atom, R¹⁵ is a methyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX153).

The compound (L-1), wherein R¹² is a hydrogen atom, R¹³ is a hydrogen atom, R¹⁴ is a fluorine atom, R¹⁵ is a hydrogen atom, R¹⁶ is a methyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX154).

The compound (L-1), wherein R¹² is a hydrogen atom, R¹³ is a hydrogen atom, R¹⁴ is a methyl group, R¹⁵ is a fluorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX155).

The compound (L-1), wherein R¹² is a hydrogen atom, R¹³ is a hydrogen atom, R¹⁴ is a methyl group, R¹⁵ is a hydrogen atom, R¹⁶ is a fluorine atom, X is an oxygen atom, and the combination of R¹, R², R³, R⁴, R⁵, R⁶, R⁷, R³, R⁹, R¹⁰, and R¹¹ is any one of the combinations shown in [Table L1] to [Table L13] (hereinafter, referred to as a compound group SX156).

The compound (L-1), wherein R¹² is a hydrogen atom, R¹³ is a hydrogen atom, R¹⁴ is a hydrogen atom, R¹⁵ is a fluorine atom, R¹⁶ is a methyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX157).

The compound (L-1), wherein R¹² is a hydrogen atom, R¹³ is a hydrogen atom, R¹⁴ is a hydrogen atom, R¹⁵ is a methyl group, R¹⁶ is a fluorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX158).

The compound (L-1), wherein R¹² is a hydrogen atom, R¹³ is a fluorine atom, R¹⁴ is a methoxy group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX159).

The compound (L-1), wherein R¹² is a hydrogen atom, R¹³ is a fluorine atom, R¹⁴ is a hydrogen atom, R¹⁵ is a methoxy group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX160).

The compound (L-1), wherein R¹² is a hydrogen atom, R¹³ is a fluorine atom, R¹⁴ is a hydrogen atom, R¹⁵ is a hydrogen atom, R¹⁶ is a methoxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX161).

The compound (L-1), wherein R¹² is a hydrogen atom, R¹³ is a methoxy group, R¹⁴ is a fluorine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX162).

The compound (L-1), wherein R¹² is a hydrogen atom, R¹³ is a methoxy group, R¹⁴ is a hydrogen atom, R¹⁵ is a fluorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX163).

The compound (L-1), wherein R¹² is a hydrogen atom, R¹³ is a methoxy group, R¹⁴ is a hydrogen atom, R¹⁵ is a hydrogen atom, R¹⁶ is a fluorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX164).

The compound (L-1), wherein R¹² is a hydrogen atom, R¹³ is a hydrogen atom, R¹⁴ is a fluorine atom, R¹⁵ is a methoxy group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX165).

The compound (L-1), wherein R¹² is a hydrogen atom, R¹³ is a hydrogen atom, R¹⁴ is a fluorine atom, R¹⁵ is a hydrogen atom, R¹⁶ is a methoxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX166).

The compound (L-1), wherein R¹² is a hydrogen atom, R¹³ is a hydrogen atom, R¹⁴ is a methoxy group, R¹⁵ is a fluorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX167).

The compound (L-1), wherein R¹² is a hydrogen atom, R¹³ is a hydrogen atom, R¹⁴ is a methoxy group, R¹⁵ is a hydrogen atom, R¹⁶ is a fluorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX168).

The compound (L-1), wherein wherein R¹² is a hydrogen atom, R¹³ is a hydrogen atom, R¹⁴ is a hydrogen atom, R¹⁵ is a fluorine atom, R¹⁶ is a methoxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX169).

The compound (L-1), wherein R¹² is a hydrogen atom, R¹³ is a hydrogen atom, R¹⁴ is a hydrogen atom, R¹⁵ is a methoxy group, R¹⁶ is a fluorine group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX170).

The compound (L-1), wherein R¹² is a hydrogen atom, R¹³ is a chlorine atom, R¹⁴ is a methyl group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX171).

The compound (L-1), wherein R¹² is a hydrogen atom, R¹³ is a chlorine atom, R¹⁴ is a hydrogen atom, R¹⁵ is a methyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX172).

The compound (L-1), wherein R¹² is a hydrogen atom, R¹³ is a chlorine atom, R¹⁴ is a hydrogen atom, R¹⁵ is a hydrogen atom, R¹⁶ is a methyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX173).

The compound (L-1), wherein R¹² is a hydrogen atom, R¹³ is a methyl group, R¹⁴ is a chlorine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX174).

The compound (L-1), wherein R¹² is a hydrogen atom, R¹³ is a methyl group, R¹⁴ is a hydrogen atom, R¹⁵ is a chlorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX175).

The compound (L-1), wherein R¹² is a hydrogen atom, R¹³ is a methyl group, R¹⁴ is a hydrogen atom, R¹⁵ is a hydrogen atom, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX176).

The compound (L-1), wherein R¹² is a hydrogen atom, R¹³ is a hydrogen atom, R¹⁴ is a chlorine atom, R¹⁵ is a methyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX177).

The compound (L-1), wherein R¹² is a hydrogen atom, R¹³ is a hydrogen atom, R¹⁴ is a chlorine atom, R¹⁵ is a hydrogen atom, R¹⁶ is a methyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX178).

The compound (L-1), wherein R¹² is a hydrogen atom, R¹³ is a hydrogen atom, R¹⁴ is a methyl group, R¹⁵ is a chlorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX179).

The compound (L-1), wherein R¹² is a hydrogen atom, R¹³ is a hydrogen atom, R¹⁴ is a methyl group, R¹⁵ is a hydrogen atom, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX180).

The compound (L-1), wherein R¹² is a hydrogen atom, R¹³ is a hydrogen atom, R¹⁴ is a hydrogen atom, R¹⁵ is a chlorine atom, R¹⁶ is a methyl group, X is an oxygen atom, and the combination of R¹, R², R³, R⁴, R⁵, R⁶, R⁷, R⁸, R⁹, R¹⁰, and R¹ is any one of the combinations shown in [Table L1] to [Table L13] (hereinafter, referred to as a compound group SX181).

The compound (L-1), wherein R¹² is a hydrogen atom, R¹³ is a hydrogen atom, R¹⁴ is a hydrogen atom, R¹⁵ is a methyl group, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX182).

The compound (L-1), wherein R¹² is a hydrogen atom, R¹³ is a chlorine atom, R¹⁴ is a methoxy group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX183).

The compound (L-1), wherein R¹² is a hydrogen atom, R¹³ is a chlorine atom, R¹⁴ is a hydrogen atom, R¹⁵ is a methoxy group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX184).

The compound (L-1), wherein R¹² is a hydrogen atom, R¹³ is a chlorine atom, R¹⁴ is a hydrogen atom, R¹⁵ is a hydrogen atom, R¹⁶ is a methoxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX185).

The compound (L-1), wherein R¹² is a hydrogen atom, R¹³ is a methoxy group, R¹⁴ is a chlorine atom, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX186).

The compound (L-1), wherein R¹² is a hydrogen atom, R¹³ is a methoxy group, R¹⁴ is a hydrogen atom, R¹⁵ is a chlorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX187).

The compound (L-1), wherein R¹² is a hydrogen atom, R¹³ is a methoxy group, R¹⁴ is a hydrogen atom, R¹⁵ is a hydrogen atom, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX188).

The compound (L-1), wherein R¹² is a hydrogen atom, R¹³ is a hydrogen atom, R¹⁴ is a chlorine atom, R¹⁵ is a methoxy group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX189).

The compound (L-1), wherein R¹² is a hydrogen atom, R¹³ is a hydrogen atom, R¹⁴ is a chlorine atom, R¹⁵ is a hydrogen atom, R¹⁶ is a methoxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX190).

The compound (L-1), wherein R¹² is a hydrogen atom, R¹³ is a hydrogen atom, R¹⁴ is a methoxy group, R¹⁵ is a chlorine atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX191).

The compound (L-1), wherein R¹² is a hydrogen atom, R¹³ is a hydrogen atom, R¹⁴ is a methoxy group, R¹⁵ is a hydrogen atom, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX192).

The compound (L-1), wherein R¹² is a hydrogen atom, R¹³ is a hydrogen atom, R¹⁴ is a hydrogen atom, R¹⁵ is a chlorine atom, R¹⁶ is a methoxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX193).

The compound (L-1), wherein R¹² is a hydrogen atom, R¹³ is a hydrogen atom, R¹⁴ is a hydrogen atom, R¹⁵ is a methoxy group, R¹⁶ is a chlorine atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX194).

The compound (L-1), wherein R¹² is a hydrogen atom, R¹³ is a methyl group, R¹⁴ is a methoxy group, R¹⁵ is a hydrogen atom, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX195).

The compound (L-1), wherein R¹² is a hydrogen atom, R¹³ is a methyl group, R¹⁴ is a hydrogen atom, R¹⁵ is a methoxy group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX196).

The compound (L-1), wherein R¹² is a hydrogen atom, R¹³ is a methyl group, R¹⁴ is a hydrogen atom, R¹⁵ is a hydrogen atom, R¹⁶ is a methoxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX197).

The compound (L-1), wherein R¹² is a hydrogen atom, R¹³ is a methoxy group, R¹⁴ is a methyl group, R¹⁵ is a hydrogen atom, R¹⁶ is a methoxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX198).

The compound (L-1), wherein R¹² is a hydrogen atom, R¹³ is a methoxy group, R¹⁴ is a hydrogen atom, R¹⁵ is a methyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX199).

The compound (L-1), wherein R¹² is a hydrogen atom, R¹³ is a methoxy group, R¹⁴ is a hydrogen atom, R¹⁵ is a hydrogen atom, R¹⁶ is a methyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX200).

The compound (L-1), wherein R¹² is a hydrogen atom, R¹³ is a hydrogen atom, R¹⁴ is a methyl group, R¹⁵ is a methoxy group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX201).

The compound (L-1), wherein R¹² is a hydrogen atom, R¹³ is a hydrogen atom, R¹⁴ is a methyl group, R¹⁵ is a hydrogen atom, R¹⁶ is a methoxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX202).

The compound (L-1), wherein R¹² is a hydrogen atom, R¹³ is a hydrogen atom, R¹⁴ is a methoxy group, R¹⁵ is a methyl group, R¹⁶ is a hydrogen atom, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX203).

The compound (L-1), wherein R¹² is a hydrogen atom, R¹³ is a hydrogen atom, R¹⁴ is a methoxy group, R¹⁵ is a hydrogen atom, R¹⁶ is a methyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX204).

The compound (L-1), wherein R¹² is a hydrogen atom, R¹³ is a hydrogen atom, R¹⁴ is a hydrogen atom, R¹⁵ is a methyl group, R¹⁶ is a methoxy group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX205).

The compound (L-1), wherein R¹² is a hydrogen atom, R¹³ is a hydrogen atom, R¹⁴ is a hydrogen atom, R¹⁵ is a methoxy group, R¹⁶ is a methyl group, X is an oxygen atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX206).

The compound (L-1), wherein R¹² is a hydrogen atom, R¹³ is a hydrogen atom, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX207).

The compound (L-1), wherein R¹² is a methyl group, R¹³ is a hydrogen atom, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX208).

The compound (L-1), wherein R¹² is a hydrogen atom, R¹³ is a methyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX209).

The compound (L-1), wherein R¹² is a hydrogen atom, R¹³ is a hydrogen atom, R¹⁴ is a methyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX210).

The compound (L-1), wherein R¹² is a hydrogen atom, R¹³ is a hydrogen atom, R¹⁴ is a hydrogen atom, R¹⁵ is a methyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX211).

The compound (L-1), wherein R¹² is a hydrogen atom, R¹³ is a hydrogen atom, R¹⁴ is a hydrogen atom, R¹⁵ is a hydrogen atom, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX212).

The compound (L-1), wherein R¹² is a hydrogen atom, R¹³ is a methyl group, R¹⁴ is a methyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX213).

The compound (L-1), wherein R¹² is a hydrogen atom, R¹³ is a methyl group, R¹⁴ is a hydrogen atom, R¹⁵ is a methyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX214).

The compound (L-1), wherein R¹² is a hydrogen atom, R¹³ is a methyl group, R¹⁴ is a hydrogen atom, R¹⁵ is a hydrogen atom, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX215).

The compound (L-1), wherein R¹² is a hydrogen atom, R¹³ is a hydrogen atom, R¹⁴ is a methyl group, R¹⁵ is a methyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX216).

The compound (L-1), wherein R¹² is a hydrogen atom, R¹³ is a hydrogen atom, R¹⁴ is a methyl group, R¹⁵ is a hydrogen atom, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX217).

The compound (L-1), wherein R¹² is a hydrogen atom, R¹³ is a hydrogen atom, R¹⁴ is a hydrogen atom, R¹⁵ is a methyl group, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX218).

The compound (L-1), wherein R¹² is a hydrogen atom, R¹³ is a methyl group, R¹⁴ is a methyl group, R¹⁵ is a methyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX219).

The compound (L-1), wherein R¹² is a hydrogen atom, R¹³ is a hydrogen atom, R¹⁴ is a methyl group, R¹⁵ is a methyl group, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX220).

The compound (L-1), wherein R¹² is a hydrogen atom, R¹³ is a fluorine atom, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX221).

The compound (L-1), wherein R¹² is a hydrogen atom, R¹³ is a hydrogen atom, R¹⁴ is a fluorine atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX222).

The compound (L-1), wherein R¹² is a hydrogen atom, R¹³ is a hydrogen atom, R¹⁴ is a hydrogen atom, R¹⁵ is a fluorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX223).

The compound (L-1), wherein R¹² is a hydrogen atom, R¹³ is a hydrogen atom, R¹⁴ is a hydrogen atom, R¹⁵ is a hydrogen atom, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX224).

The compound (L-1), wherein R¹² is a hydrogen atom, R¹³ is a chlorine atom, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX225).

The compound (L-1), wherein R¹² is a hydrogen atom, R¹³ is a hydrogen atom, R¹⁴ is a chlorine atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX226).

The compound (L-1), wherein R¹² is a hydrogen atom, R¹³ is a hydrogen atom, R¹⁴ is a hydrogen atom, R¹⁵ is a chlorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX227).

The compound (L-1), wherein R¹² is a hydrogen atom, R¹³ is a hydrogen atom, R¹⁴ is a hydrogen atom, R¹⁵ is a hydrogen atom, R¹⁶ is a chlorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX228).

The compound (L-1), wherein R¹² is a hydrogen atom, R¹³ is a bromine atom, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX229).

The compound (L-1), wherein R¹² is a hydrogen atom, R¹³ is a hydrogen atom, R¹⁴ is a bromine atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX230).

The compound (L-1), wherein R¹² is a hydrogen atom, R¹³ is a hydrogen atom, R¹⁴ is a hydrogen atom, R¹⁵ is a bromine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX231).

The compound (L-1), wherein R¹² is a hydrogen atom, R¹³ is a hydrogen atom, R¹⁴ is a hydrogen atom, R¹⁵ is a hydrogen atom, R¹⁶ is a bromine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX232).

The compound (L-1), wherein R¹² is a hydrogen atom, R¹³ is an iodine atom, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX233).

The compound (L-1), wherein R¹² is a hydrogen atom, R¹³ is a hydrogen atom, R¹⁴ is an iodine atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX234).

The compound (L-1), wherein R¹² is a hydrogen atom, R¹³ is a hydrogen atom, R¹⁴ is a hydrogen atom, R¹⁵ is an iodine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX235).

The compound (L-1), wherein R¹² is a hydrogen atom, R¹³ is a hydrogen atom, R¹⁴ is a hydrogen atom, R¹⁵ is a hydrogen atom, R¹⁶ is an iodine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX236).

The compound (L-1), wherein R¹² is a hydrogen atom, R¹³ is a methoxy group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX237).

The compound (L-1), wherein R¹² is a hydrogen atom, R¹³ is a hydrogen atom, R¹⁴ is a methoxy group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX238).

The compound (L-1), wherein R¹² is a hydrogen atom, R¹³ is a hydrogen atom, R¹⁴ is a hydrogen atom, R¹⁵ is a methoxy group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX239).

The compound (L-1), wherein R¹² is a hydrogen atom, R¹³ is a hydrogen atom, R¹⁴ is a hydrogen atom, R¹⁵ is a hydrogen atom, R¹⁶ is a methoxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX240).

The compound (L-1), wherein R¹² is a hydrogen atom, R¹³ is a trifluoromethyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX241).

The compound (L-1), wherein R¹² is a hydrogen atom, R¹³ is a hydrogen atom, R¹⁴ is a trifluoromethyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX242).

The compound (L-1), wherein R¹² is a hydrogen atom, R¹³ is a hydrogen atom, R¹⁴ is a hydrogen atom, R¹⁵ is a trifluoromethyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX243).

The compound (L-1), wherein R¹² is a hydrogen atom, R¹³ is a hydrogen atom, R¹⁴ is a hydrogen atom, R¹⁵ is a hydrogen atom, R¹⁶ is a trifluoromethyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX244).

The compound (L-1), wherein R¹² is a hydrogen atom, R¹³ is a cyclopropyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX245).

The compound (L-1), wherein R¹² is a hydrogen atom, R¹³ is a hydrogen atom, R¹⁴ is a cyclopropyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX246).

The compound (L-1), wherein R¹² is a hydrogen atom, R¹³ is a hydrogen atom, R¹⁴ is a hydrogen atom, R¹⁵ is a cyclopropyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX247).

The compound (L-1), wherein R¹² is a hydrogen atom, R¹³ is a hydrogen atom, R¹⁴ is a hydrogen atom, R¹⁵ is a hydrogen atom, R¹⁶ is a cyclopropyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX248).

The compound (L-1), wherein R¹² is a hydrogen atom, R¹³ is a cyclobutyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX249).

The compound (L-1), wherein R¹² is a hydrogen atom, R¹³ is a hydrogen atom, R¹⁴ is a cyclobutyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX250).

The compound (L-1), wherein R¹² is a hydrogen atom, R¹³ is a hydrogen atom, R¹⁴ is a hydrogen atom, R¹⁵ is a cyclobutyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX251).

The compound (L-1), wherein R¹² is a hydrogen atom, R¹³ is a hydrogen atom, R¹⁴ is a hydrogen atom, R¹⁵ is a hydrogen atom, R¹⁶ is a cyclobutyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX252).

The compound (L-1), wherein R¹² is a hydrogen atom, R¹³ is a cyclopentyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX253).

The compound (L-1), wherein R¹² is a hydrogen atom, R¹³ is a hydrogen atom, R¹⁴ is a cyclopentyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX254).

The compound (L-1), wherein R¹² is a hydrogen atom, R¹³ is a hydrogen atom, R¹⁴ is a hydrogen atom, R¹⁵ is a cyclopentyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX255).

The compound (L-1), wherein R¹² is a hydrogen atom, R¹³ is a hydrogen atom, R¹⁴ is a hydrogen atom, R¹⁵ is a hydrogen atom, R¹⁶ is a cyclopentyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX256).

The compound (L-1), wherein R¹² is a hydrogen atom, R¹³ is a cyclohexyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX257).

The compound (L-1), wherein R¹² is a hydrogen atom, R¹³ is a hydrogen atom, R¹⁴ is a cyclohexyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX258).

The compound (L-1), wherein R¹² is a hydrogen atom, R¹³ is a hydrogen atom, R¹⁴ is a hydrogen atom, R¹⁵ is a cyclohexyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX259).

The compound (L-1), wherein R¹² is a hydrogen atom, R¹³ is a hydrogen atom, R¹⁴ is a hydrogen atom, R¹⁵ is a hydrogen atom, R¹⁶ is a cyclohexyl atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX260).

The compound (L-1), wherein R¹² is a hydrogen atom, R¹³ is a phenyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX261).

The compound (L-1), wherein wherein R¹² is a hydrogen atom, R¹³ is a hydrogen atom, R¹⁴ is a phenyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX262).

The compound (L-1), wherein R¹² is a hydrogen atom, R¹³ is a hydrogen atom, R¹⁴ is a hydrogen atom, R¹⁵ is a phenyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX263).

The compound (L-1), wherein R¹² is a hydrogen atom, R¹³ is a hydrogen atom, R¹⁴ is a hydrogen atom, R¹⁵ is a hydrogen atom, R¹⁶ is a phenyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX264).

The compound (L-1), wherein R¹² is a hydrogen atom, R¹³ is an acetyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX265).

The compound (L-1), wherein R¹² is a hydrogen atom, R¹³ is a hydrogen atom, R¹⁴ is an acetyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX266).

The compound (L-1), wherein R¹² is a hydrogen atom, R¹³ is a hydrogen atom, R¹⁴ is a hydrogen atom, R¹⁵ is an acetyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX267).

The compound (L-1), wherein R¹² is a hydrogen atom, R¹³ is a hydrogen atom, R¹⁴ is a hydrogen atom, R¹⁵ is a hydrogen atom, R¹⁶ is an acetyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX268).

The compound (L-1), wherein R¹² is a hydrogen atom, R¹³ is a trifluoroacetyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX269).

The compound (L-1), wherein R¹² is a hydrogen atom, R¹³ is a hydrogen atom, R¹⁴ is a trifluoroacetyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX270).

The compound (L-1), wherein R¹² is a hydrogen atom, R¹³ is a hydrogen atom, R¹⁴ is a hydrogen atom, R¹⁵ is a trifluoroacetyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX271).

The compound (L-1), wherein R¹² is a hydrogen atom, R¹³ is a hydrogen atom, R¹⁴ is a hydrogen atom, R¹⁵ is a hydrogen atom, R¹⁶ is a trifluoroacetyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX272).

The compound (L-1), wherein R¹² is a hydrogen atom, R¹³ is a methoxycarbonyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX273).

The compound (L-1), wherein R¹² is a hydrogen atom, R¹³ is a hydrogen atom, R¹⁴ is a methoxycarbonyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX274).

The compound (L-1), wherein R¹² is a hydrogen atom, R¹³ is a hydrogen atom, R¹⁴ is a hydrogen atom, R¹⁵ is a methoxycarbonyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX275).

The compound (L-1), wherein R¹² is a hydrogen atom, R¹³ is a hydrogen atom, R¹⁴ is a hydrogen atom, R¹⁵ is a hydrogen atom, R¹⁶ is a methoxycarbonyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX276).

The compound (L-1), wherein R¹² is a hydrogen atom, R¹³ is a trifluoromethoxycarbonyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX277).

The compound (L-1), wherein R¹² is a hydrogen atom, R¹³ is a hydrogen atom, R¹⁴ is a trifluoromethoxycarbonyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX278).

The compound (L-1), wherein R¹² is a hydrogen atom, R¹³ is a hydrogen atom, R¹⁴ is a hydrogen atom, R¹⁵ is a trifluoromethoxycarbonyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX279).

The compound (L-1), wherein R¹² is a hydrogen atom, R¹³ is a hydrogen atom, R¹⁴ is a hydrogen atom, R¹⁵ is a hydrogen atom, R¹⁶ is a trifluoromethoxycarbonyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX280).

The compound (L-1), wherein R¹² is a hydrogen atom, R¹³ is an acetoxy group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX281).

The compound (L-1), wherein R¹² is a hydrogen atom, R¹³ is a hydrogen atom, R¹⁴ is an acetoxy group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX282).

The compound (L-1), wherein R¹² is a hydrogen atom, R¹³ is a hydrogen atom, R¹⁴ is a hydrogen atom, R¹⁵ is an acetoxy group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX283).

The compound (L-1), wherein R¹² is a hydrogen atom, R¹³ is a hydrogen atom, R¹⁴ is a hydrogen atom, R¹⁵ is a hydrogen atom, R¹⁶ is an acetoxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX284).

The compound (L-1), wherein R¹² is a hydrogen atom, R¹³ is a trifluoroacetoxy group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX285).

The compound (L-1), wherein R¹² is a hydrogen atom, R¹³ is a hydrogen atom, R¹⁴ is a trifluoroacetoxy group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX286).

The compound (L-1), wherein R¹² is a hydrogen atom, R¹³ is a hydrogen atom, R¹⁴ is a hydrogen atom, R¹⁵ is a trifluoroacetoxy group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX287).

The compound (L-1), wherein R¹² is a hydrogen atom, R¹³ is a hydrogen atom, R¹⁴ is a hydrogen atom, R¹⁵ is a hydrogen atom, R¹⁶ is a trifluoroacetoxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX288).

The compound (L-1), wherein R¹² is a hydrogen atom, R¹³ is a cyano group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX289).

The compound (L-1), wherein R¹² is a hydrogen atom, R¹³ is a hydrogen atom, R¹⁴ is a cyano group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX290).

The compound (L-1), wherein R¹² is a hydrogen atom, R¹³ is a hydrogen atom, R¹⁴ is a hydrogen atom, R¹⁵ is a cyano group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX291).

The compound (L-1), wherein R¹² is a hydrogen atom, R¹³ is a hydrogen atom, R¹⁴ is a hydrogen atom, R¹⁵ is a hydrogen atom, R¹⁶ is a cyano group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX292).

The compound (L-1), wherein R¹² is a hydrogen atom, R¹³ is a formyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX293).

The compound (L-1), wherein R¹² is a hydrogen atom, R¹³ is a hydrogen atom, R¹⁴ is a formyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX294).

The compound (L-1), wherein R¹² is a hydrogen atom, R¹³ is a hydrogen atom, R¹⁴ is a hydrogen atom, R¹⁵ is a formyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX295).

The compound (L-1), wherein R¹² is a hydrogen atom, R¹³ is a hydrogen atom, R¹⁴ is a hydrogen atom, R¹⁵ is a hydrogen atom, R¹⁶ is a formyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX296).

The compound (L-1), wherein R¹² is a hydrogen atom, R¹³ is a carboxy group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX297).

The compound (L-1), wherein R¹² is a hydrogen atom, R¹³ is a hydrogen atom, R¹⁴ is a carboxy group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX298).

The compound (L-1), wherein R¹² is a hydrogen atom, R¹³ is a hydrogen atom, R¹⁴ is a hydrogen atom, R¹⁵ is a carboxy group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX299).

The compound (L-1), wherein R¹² is a hydrogen atom, R¹³ is a hydrogen atom, R¹⁴ is a hydrogen atom, R¹⁵ is a hydrogen atom, R¹⁶ is a carboxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX300).

The compound (L-1), wherein R¹² is a hydrogen atom, R¹³ is a nitro group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX301).

The compound (L-1), wherein R¹² is a hydrogen atom, R¹³ is a hydrogen atom, R¹⁴ is a nitro group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX302).

The compound (L-1), wherein R¹² is a hydrogen atom, R¹³ is a hydrogen atom, R¹⁴ is a hydrogen atom, R¹⁵ is a nitro group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX303).

The compound (L-1), wherein R¹² is a hydrogen atom, R¹³ is a hydrogen atom, R¹⁴ is a hydrogen atom, R¹⁵ is a hydrogen atom, R¹⁶ is a nitro group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX304).

The compound (L-1), wherein R¹² is a hydrogen atom, R¹³ is a hydroxy group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX305).

The compound (L-1), wherein R¹² is a hydrogen atom, R¹³ is a hydrogen atom, R¹⁴ is a hydroxy group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX306).

The compound (L-1), wherein R¹² is a hydrogen atom, R¹³ is a hydrogen atom, R¹⁴ is a hydrogen atom, R¹⁵ is a hydroxy group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX307).

The compound (L-1), wherein R¹² is a hydrogen atom, R¹³ is a hydrogen atom, R¹⁴ is a hydrogen atom, R¹⁵ is a hydrogen atom, R¹⁶ is a hydroxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX308).

The compound (L-1), wherein R¹² is a hydrogen atom, R¹³ is a fluorine atom, R¹⁴ is a fluorine atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX309).

The compound (L-1), wherein R¹² is a hydrogen atom, R¹³ is a fluorine atom, R¹⁴ is a hydrogen atom, R¹⁵ is a fluorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX310).

The compound (L-1), wherein R¹² is a hydrogen atom, R¹³ is a fluorine atom, R¹⁴ is a hydrogen atom, R¹⁵ is a hydrogen atom, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX311).

The compound (L-1), wherein R¹² is a hydrogen atom, R¹³ is a hydrogen atom, R¹⁴ is a fluorine atom, R¹⁵ is a fluorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX312).

The compound (L-1), wherein R¹² is a hydrogen atom, R¹³ is a hydrogen atom, R¹⁴ is a fluorine atom, R¹⁵ is a hydrogen atom, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX313).

The compound (L-1), wherein R¹² is a hydrogen atom, R¹³ is a hydrogen atom, R¹⁴ is a hydrogen atom, R¹⁵ is a fluorine atom, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX314).

The compound (L-1), wherein R¹² is a hydrogen atom, R¹³ is a fluorine atom, R¹⁴ is a fluorine atom, R¹⁵ is a fluorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX315).

The compound (L-1), wherein R¹² is a hydrogen atom, R¹³ is a hydrogen atom, R¹⁴ is a fluorine atom, R¹⁵ is a fluorine atom, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX316).

The compound (L-1), wherein R¹² is a hydrogen atom, R¹³ is a chlorine atom, R¹⁴ is a chlorine atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX317).

The compound (L-1), wherein R¹² is a hydrogen atom, R¹³ is a chlorine atom, R¹⁴ is a hydrogen atom, R¹⁵ is a chlorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX318).

The compound (L-1), wherein R¹² is a hydrogen atom, R¹³ is a chlorine atom, R¹⁴ is a hydrogen atom, R¹⁵ is a hydrogen atom, R¹⁶ is a chlorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX319).

The compound (L-1), wherein R¹² is a hydrogen atom, R¹³ is a hydrogen atom, R¹⁴ is a chlorine atom, R¹⁵ is a chlorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX320).

The compound (L-1), wherein R¹² is a hydrogen atom, R¹³ is a hydrogen atom, R¹⁴ is a chlorine atom, R¹⁵ is a hydrogen atom, R¹⁶ is a chlorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX321).

The compound (L-1), wherein R¹² is a hydrogen atom, R¹³ is a hydrogen atom, R¹⁴ is a hydrogen atom, R¹⁵ is a chlorine atom, R¹⁶ is a chlorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX322).

The compound (L-1), wherein R¹² is a hydrogen atom, R¹³ is a chlorine atom, R¹⁴ is a chlorine atom, R¹⁵ is a chlorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX323).

The compound (L-1), wherein R¹² is a hydrogen atom, R¹³ is a hydrogen atom, R¹⁴ is a chlorine atom, R¹⁵ is a chlorine atom, R¹⁶ is a chlorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX324).

The compound (L-1), wherein R¹² is a hydrogen atom, R¹³ is a bromine atom, R¹⁴ is a bromine atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX325).

The compound (L-1), wherein R¹² is a hydrogen atom, R¹³ is a bromine atom, R¹⁴ is a hydrogen atom, R¹⁵ is a bromine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX326).

The compound (L-1), wherein R¹² is a hydrogen atom, R¹³ is a bromine atom, R¹⁴ is a hydrogen atom, R¹⁵ is a hydrogen atom, R¹⁶ is a bromine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX327).

The compound (L-1), wherein R¹² is a hydrogen atom, R¹³ is a hydrogen atom, R¹⁴ is a bromine atom, R¹⁵ is a bromine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX328).

The compound (L-1), wherein R¹² is a hydrogen atom, R¹³ is a hydrogen atom, R¹⁴ is a bromine atom, R¹⁵ is a hydrogen atom, R¹⁶ is a bromine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX329).

The compound (L-1), wherein R¹² is a hydrogen atom, R¹³ is a hydrogen atom, R¹⁴ is a hydrogen atom, R¹⁵ is a bromine atom, R¹⁶ is a bromine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX330).

The compound (L-1), wherein R¹² is a hydrogen atom, R¹³ is a bromine atom, R¹⁴ is a bromine atom, R¹⁵ is a bromine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX331).

The compound (L-1), wherein R¹² is a hydrogen atom, R¹³ is a hydrogen atom, R¹⁴ is a bromine atom, R¹⁵ is a bromine atom, R¹⁶ is a bromine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX332).

The compound (L-1), wherein R¹² is a hydrogen atom, R¹³ is an iodine atom, R¹⁴ is an iodine atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX333).

The compound (L-1), wherein R¹² is a hydrogen atom, R¹³ is an iodine atom, R¹⁴ is a hydrogen atom, R¹⁵ is an iodine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX334).

The compound (L-1), wherein R¹² is a hydrogen atom, R¹³ is an iodine atom, R¹⁴ is a hydrogen atom, R¹⁵ is a hydrogen atom, R¹⁶ is an iodine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX335).

The compound (L-1), wherein R¹² is a hydrogen atom, R¹³ is a hydrogen atom, R¹⁴ is an iodine atom, R¹⁵ is an iodine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX336).

The compound (L-1), wherein R¹² is a hydrogen atom, R¹³ is a hydrogen atom, R¹⁴ is an iodine atom, R¹⁵ is a hydrogen atom, R¹⁶ is an iodine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX337).

The compound (L-1), wherein R¹² is a hydrogen atom, R¹³ is a hydrogen atom, R¹⁴ is a hydrogen atom, R¹⁵ is an iodine atom, R¹⁶ is an iodine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX338).

The compound (L-1), wherein R¹² is a hydrogen atom, R¹³ is an iodine atom, R¹⁴ is an iodine atom, R¹⁵ is an iodine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX339).

The compound (L-1), wherein R¹² is a hydrogen atom, R¹³ is a hydrogen atom, R¹⁴ is an iodine atom, R¹⁵ is an iodine atom, R¹⁶ is an iodine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX340).

The compound (L-1), wherein R¹² is a hydrogen atom, R¹³ is a fluorine atom, R¹⁴ is a chlorine atom, R¹⁵ is a hydrogen atom, R¹⁶ is an iodine atom, X is a sulfur atom, and and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX341).

The compound (L-1), wherein R¹² is a hydrogen atom, R¹³ is a fluorine atom, R¹⁴ is a hydrogen atom, R¹⁵ is a chlorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX342).

The compound (L-1), wherein R¹² is a hydrogen atom, R¹³ is a fluorine atom, R¹⁴ is a hydrogen atom, R¹⁵ is a hydrogen atom, R¹⁶ is a chlorine atom, X is a sulfur atom, and the combination of R¹, R², R³, R⁴, R⁵, R⁶, R⁷, R⁸, R⁹, R¹⁰, and R¹ is any one of the combinations shown in [Table L1] to [Table L13] (hereinafter, referred to as a compound group SX343).

The compound (L-1), wherein R¹² is a hydrogen atom, R¹³ is a chlorine atom, R¹⁴ is a fluorine atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX344).

The compound (L-1), wherein R¹² is a hydrogen atom, R¹³ is a chlorine atom, R¹⁴ is a hydrogen atom, R¹⁵ is a fluorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX345).

The compound (L-1), wherein R¹² is a hydrogen atom, R¹³ is a chlorine atom, R¹⁴ is a hydrogen atom, R¹⁵ is a hydrogen atom, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX346).

The compound (L-1), wherein R¹² is a hydrogen atom, R¹³ is a hydrogen atom, R¹⁴ is a fluorine atom, R¹⁵ is a chlorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX347).

The compound (L-1), wherein R¹² is a hydrogen atom, R¹³ is a hydrogen atom, R¹⁴ is a fluorine atom, R¹⁵ is a iodine atom, R¹⁶ is an iodine atom, X is a sulfur atom, and hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX348).

The compound (L-1), wherein R¹² is a hydrogen atom, R¹³ is a hydrogen atom, R¹⁴ is a chlorine atom, R¹⁵ is a fluorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX349).

The compound (L-1), wherein R¹² is a hydrogen atom, R¹³ is a hydrogen atom, R¹⁴ is a chlorine atom, R¹⁵ is a hydrogen atom, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX350).

The compound (L-1), wherein R¹² is a hydrogen atom, R¹³ is a hydrogen atom, R¹⁴ is a hydrogen atom, R¹⁵ is a fluorine atom, R¹⁶ is a chlorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX351).

The compound (L-1), wherein R¹² is a hydrogen atom, R¹³ is a hydrogen atom, R¹⁴ is a hydrogen atom, R¹⁵ is a chlorine atom, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX352).

The compound (L-1), wherein R¹² is a hydrogen atom, R¹³ is a fluorine atom, R¹⁴ is a methyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX353).

The compound (L-1), wherein R¹² is a hydrogen atom, R¹³ is a fluorine atom, R¹⁴ is a hydrogen atom, R¹⁵ is a methyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX354).

The compound (L-1), wherein R¹² is a hydrogen atom, R¹³ is a fluorine atom, R¹⁴ is a hydrogen atom, R¹⁵ is a hydrogen atom, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX355).

The compound (L-1), wherein R¹² is a hydrogen atom, R¹³ is a methyl group, R¹⁴ is a fluorine atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX356).

The compound (L-1), wherein R¹² is a hydrogen atom, R¹³ is a methyl group, R¹⁴ is a hydrogen atom, R¹⁵ is a fluorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX357).

The compound (L-1), wherein R¹² is a hydrogen atom, R¹³ is a methyl group, R¹⁴ is a hydrogen atom, R¹⁵ is a hydrogen atom, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX358).

The compound (L-1), wherein R¹² is a hydrogen atom, R¹³ is a hydrogen atom, R¹⁴ is a fluorine atom, R¹⁵ is a methyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX359).

The compound (L-1), wherein R¹² is a hydrogen atom, R¹³ is a hydrogen atom, R¹⁴ is a fluorine atom, R¹⁵ is a hydrogen atom, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX360).

The compound (L-1), wherein R¹² is a hydrogen atom, R¹³ is a hydrogen atom, R¹⁴ is a methyl group, R¹⁵ is a fluorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX361).

The compound (L-1), wherein R¹² is a hydrogen atom, R¹³ is a hydrogen atom, R¹⁴ is a methyl group, R¹⁵ is a hydrogen atom, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX362).

The compound (L-1), wherein R¹² is a hydrogen atom, R¹³ is a hydrogen atom, R¹⁴ is a hydrogen atom, R¹⁵ is a fluorine atom, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX363).

The compound (L-1), wherein R¹² is a hydrogen atom, R¹³ is a hydrogen atom, R¹⁴ is a hydrogen atom, R¹⁵ is a methyl group, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX364).

The compound (L-1), wherein R¹² is a hydrogen atom, R¹³ is a fluorine atom, R¹⁴ is a methoxy group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX365).

The compound (L-1), wherein R¹² is a hydrogen atom, R¹³ is a fluorine atom, R¹⁴ is a hydrogen atom, R¹⁵ is a methoxy group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX366).

The compound (1-1), wherein R¹² is a hydrogen atom, R¹³ is a fluorine atom, R¹⁴ is a hydrogen atom, R¹⁵ is a hydrogen atom, R¹⁶ is a methoxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX367).

The compound (L-1), wherein R¹² is a hydrogen atom, R¹³ is a methoxy group, R¹⁴ is a fluorine atom, R¹⁵ is a hydrogen atom, R¹⁶ is a hydrogen atom, X is a sulfur atom, and the combination of R¹, R², R³, R⁴, R⁵, R⁶, R⁷, R⁸, R³, R¹⁰, and R¹¹ is any one of the combinations shown in [Table L1] to [Table L13] (hereinafter, referred to as a compound group SX368).

The compound (L-1), wherein R¹² is a hydrogen atom, R¹³ is a methoxy group, R¹⁴ is a hydrogen atom, R¹⁵ is a fluorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX369).

The compound (L-1), wherein R¹² is a hydrogen atom, R¹³ is a methoxy group, R¹⁴ is a hydrogen atom, R¹⁵ is a hydrogen atom, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX370).

The compound (L-1), wherein R¹² is a hydrogen atom, R¹³ is a hydrogen atom, R¹⁴ is a fluorine atom, R¹⁵ is a methoxy group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX371).

The compound (L-1), wherein R¹² is a hydrogen atom, R¹³ is a hydrogen atom, R¹⁴ is a fluorine atom, R¹⁵ is a hydrogen atom, R¹⁶ is a methoxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX372).

The compound (L-1), wherein R¹² is a hydrogen atom, R¹³ is a hydrogen atom, R¹⁴ is a methoxy group, R¹⁵ is a fluorine atom, R¹⁶ is a methoxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX373).

The compound (L-1), wherein R¹² is a hydrogen atom, R¹³ is a hydrogen atom, R¹⁴ is a methoxy group, R¹⁵ is a hydrogen atom, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX374).

The compound (L-1), wherein R¹² is a hydrogen atom, R¹³ is a hydrogen atom, R¹⁴ is a hydrogen atom, R¹⁵ is a fluorine atom, R¹⁶ is a methoxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX375).

The compound (L-1), wherein R¹² is a hydrogen atom, R¹³ is a hydrogen atom, R¹⁴ is a hydrogen atom, R¹⁵ is a methoxy group, R¹⁶ is a fluorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX376).

The compound (L-1), wherein R¹² is a hydrogen atom, R¹³ is a chlorine atom, R¹⁴ is a methyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX377).

The compound (L-1), wherein R¹² is a hydrogen atom, R¹³ is a chlorine atom, R¹⁴ is a hydrogen atom, R¹⁵ is a methyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX378).

The compound (1-1), wherein R¹² is a hydrogen atom, R¹³ is a chlorine atom, R¹⁴ is a hydrogen atom, R¹⁵ is a hydrogen atom, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX379).

The compound (L-1), wherein R¹² is a hydrogen atom, R¹³ is a methyl group, R¹⁴ is a chlorine atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX380).

The compound (L-1), wherein R¹² is a hydrogen atom, R¹³ is a methyl group, R¹⁴ is a hydrogen atom, R¹⁵ is a chlorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX381).

The compound (L-1), wherein R¹² is a hydrogen atom, R¹³ is a methyl group, R¹⁴ is a hydrogen atom, R¹⁵ is a hydrogen atom, R¹⁶ is a chlorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX382).

The compound (L-1), wherein R¹² is a hydrogen atom, R¹³ is a hydrogen atom, R¹⁴ is a chlorine atom, R¹⁵ is a methyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX383).

The compound (L-1), wherein R¹² is a hydrogen atom, R¹³ is a hydrogen atom, R¹⁴ is a chlorine atom, R¹⁵ is a hydrogen atom, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX384).

The compound (L-1), wherein R¹² is a hydrogen atom, R¹³ is a hydrogen atom, R¹⁴ is a methyl group, R¹⁵ is a chlorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX385).

The compound (L-1), wherein R¹² is a hydrogen atom, R¹³ is a hydrogen atom, R¹⁴ is a methyl group, R¹⁵ is a hydrogen atom, R¹⁶ is a chlorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX386).

The compound (L-1), wherein R¹² is a hydrogen atom, R¹³ is a hydrogen atom, R¹⁴ is a hydrogen atom, R¹⁵ is a chlorine atom, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX387).

The compound (L-1), wherein R¹² is a hydrogen atom, R¹³ is a hydrogen atom, R¹⁴ is a hydrogen atom, R¹⁵ is a methyl group, R¹⁶ is a chlorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX388).

The compound (L-1), wherein R¹² is a hydrogen atom, R¹³ is a chlorine atom, R¹⁴ is a methoxy group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX389).

The compound (L-1), wherein R¹² is a hydrogen atom, R¹³ is a chlorine atom, R¹⁴ is a hydrogen atom, R¹⁵ is a methoxy group, R¹⁶ is a hydrogen atom, X is a sulfur atom, and the combination of R¹, R², R³, R⁴, R⁵, R⁶, R¹, R⁸, R⁹, R¹⁰, and R¹¹ is any one of the combinations shown in [Table L1] to [Table L13] (hereinafter, referred to as a compound group SX390).

The compound (L-1), wherein R¹² is a hydrogen atom, R¹³ is a chlorine atom, R¹⁴ is a hydrogen atom, R¹⁵ is a hydrogen atom, R¹⁶ is a methoxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX391).

The compound (L-1), wherein R¹² is a hydrogen atom, R¹³ is a methoxy group, R¹⁴ is a chlorine atom, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX392).

The compound (L-1), wherein R¹² is a hydrogen atom, R¹³ is a methoxy group, R¹⁴ is a hydrogen atom, R¹⁵ is a chlorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX393).

The compound (L-1), wherein R¹² is a hydrogen atom, R¹³ is a methoxy group, R¹⁴ is a hydrogen atom, R¹⁵ is a hydrogen atom, R¹⁶ is a chlorine atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX394).

The compound (L-1), wherein R¹² is a hydrogen atom, R¹³ is a hydrogen atom, R¹⁴ is a chlorine atom, R¹⁵ is a methoxy group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX395).

The compound (L-1), wherein R¹² is a hydrogen atom, R¹³ is a hydrogen atom, R¹⁴ is a chlorine atom, R¹⁵ is a hydrogen atom, R¹⁶ is a methoxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX396).

The compound (L-1), wherein R¹² is a hydrogen atom, R¹³ is a hydrogen atom, R¹⁴ is a methoxy group, R¹⁵ is a chlorine atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX397).

The compound (L-1), wherein R¹² is a hydrogen atom, R¹³ is a hydrogen atom, R¹⁴ is a methoxy group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX398).

The compound (L-1), wherein R¹² is a hydrogen atom, R¹³ is a hydrogen atom, R¹⁴ is a hydrogen atom, R¹⁵ is a chlorine atom, R¹⁶ is a methoxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX399).

The compound (L-1), wherein R¹² is a hydrogen atom, R¹³ is a hydrogen atom, R¹⁴ is a hydrogen atom, R¹⁵ is a methoxy group, R¹⁶ is a chlorine group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX400).

The compound (L-1), wherein R¹² is a hydrogen atom, R¹³ is a methyl group, R¹⁴ is a methoxy group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX401).

The compound (L-1), wherein R¹² is a hydrogen atom, R¹³ is a methyl group, R¹⁴ is a hydrogen atom, R¹⁵ is a methoxy group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX402).

The compound (L-1), wherein R¹² is a hydrogen atom, R¹³ is a methyl group, R¹⁴ is a hydrogen atom, R¹⁵ is a hydrogen atom, R¹⁶ is a methoxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX403).

The compound (L-1), wherein R¹² is a hydrogen atom, R¹³ is a methoxy group, R¹⁴ is a methyl group, R¹⁵ is a hydrogen atom, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX404).

The compound (L-1), wherein R¹² is a hydrogen atom, R¹³ is a methoxy group, R¹⁴ is a hydrogen atom, R¹⁵ is a methyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX405).

The compound (L-1), wherein R¹² is a hydrogen atom, R¹³ is a methoxy group, R¹⁴ is a hydrogen atom, R¹⁵ is a hydrogen atom, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX406).

The compound (L-1), wherein R¹² is a hydrogen atom, R¹³ is a hydrogen atom, R¹⁴ is a methyl group, R¹⁵ is a methoxy group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX407).

The compound (L-1), wherein R¹² is a hydrogen atom, R¹³ is a hydrogen atom, R¹⁴ is a methyl group, R¹⁵ is a hydrogen atom, R¹⁶ is a methoxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX408).

The compound (L-1), wherein R¹² is a hydrogen atom, R¹³ is a hydrogen atom, R¹⁴ is a methoxy group, R¹⁵ is a methyl group, R¹⁶ is a hydrogen atom, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX409).

The compound (L-1), wherein R¹² is a hydrogen atom, R¹³ is a hydrogen atom, R¹⁴ is a methoxy group, R¹⁵ is a hydrogen atom, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX410).

The compound (L-1), wherein R¹² is a hydrogen atom, R¹³ is a hydrogen atom, R¹⁴ is a hydrogen atom, R¹⁵ is a methyl group, R¹⁶ is a methoxy group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX411).

The compound (L-1), wherein R¹² is a hydrogen atom, R¹³ is a hydrogen atom, R¹⁴ is a hydrogen atom, R¹⁵ is a methoxy group, R¹⁶ is a methyl group, X is a sulfur atom, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX412).

The compound (L-1), wherein R¹² is a hydrogen atom, R¹³ is a hydrogen atom, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX413).

The compound (L-1), wherein R¹² is a methyl group, R¹³ is a hydrogen atom, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX414).

The compound (L-1), wherein R¹² is a hydrogen atom, R¹³ is a methyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX415).

The compound (L-1), wherein R¹² is a hydrogen atom, R¹³ is a hydrogen atom, R¹⁴ is a methyl group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX416).

The compound (L-1), wherein R¹² is a hydrogen atom, R¹³ is a hydrogen atom, R¹⁴ is a hydrogen atom, R¹⁵ is a methyl group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX417).

The compound (L-1), wherein R¹² is a hydrogen atom, R¹³ is a hydrogen atom, R¹⁴ is a hydrogen atom, R¹⁵ is a hydrogen atom, R¹⁶ is a methyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX418).

The compound (L-1), wherein R¹² is a hydrogen atom, R¹³ is a methyl group, R¹⁴ is a methyl group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX419).

The compound (L-1), wherein R¹² is a hydrogen atom, R¹³ is a methyl group, R¹⁴ is a hydrogen atom, R¹⁵ is a methyl group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX420).

The compound (L-1), wherein R¹² is a hydrogen atom, R¹³ is a methyl group, R¹⁴ is a hydrogen atom, R¹⁵ is a hydrogen atom, R¹⁶ is a methyl group, X is NH, and the combination of R¹, R², R³, B4, R⁵, R⁶, R⁷, R⁸, R⁹, R¹⁰, and R¹¹ is any one of the combinations shown in [Table L1] to [Table L13] (hereinafter, referred to as a compound group SX421).

The compound (L-1), wherein R¹² is a hydrogen atom, R¹³ is a hydrogen atom, R¹⁴ is a methyl group, R¹⁵ is a methyl group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX422).

The compound (L-1), wherein R¹² is a hydrogen atom, R¹³ is a hydrogen atom, R¹⁴ is a methyl group, R¹⁵ is a hydrogen atom, R¹⁶ is a methyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX423).

The compound (L-1), wherein R¹² is a hydrogen atom, R¹³ is a hydrogen atom, R¹⁴ is a hydrogen atom, R¹⁵ is a methyl group, R¹⁶ is a methyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX424).

The compound (L-1), wherein R¹² is a hydrogen atom, R¹³ is a methyl group, R¹⁴ is a methyl group, R¹⁵ is a methyl group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX425).

The compound (L-1), wherein R¹² is a hydrogen atom, R¹³ is a hydrogen atom, R¹⁴ is a methyl group, R¹⁵ is a methyl group, R¹⁶ is a methyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX426).

The compound (L-1), wherein R¹² is a hydrogen atom, R¹³ is a fluorine atom, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX427).

The compound (L-1), wherein R¹² is a hydrogen atom, R¹³ is a hydrogen atom, R¹⁴ is a fluorine atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX428).

The compound (L-1), wherein R¹² is a hydrogen atom, R¹³ is a hydrogen atom, R¹⁴ is a hydrogen atom, R¹⁵ is a fluorine atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX429).

The compound (L-1), wherein R¹² is a hydrogen atom, R¹³ is a hydrogen atom, R¹⁴ is a hydrogen atom, R¹⁵ is a hydrogen atom, R¹⁶ is a fluorine atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX430).

The compound (L-1), wherein R¹² is a hydrogen atom, R¹³ is a chlorine atom, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX431).

The compound (L-1), wherein R¹² is a hydrogen atom, R¹³ is a hydrogen atom, R¹⁴ is a chlorine atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX432).

The compound (L-1), wherein R¹² is a hydrogen atom, R¹³ is a hydrogen atom, R¹⁴ is a hydrogen atom, R¹⁵ is a chlorine atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX433).

The compound (L-1), wherein R¹² is a hydrogen atom, R¹³ is a hydrogen atom, R¹⁴ is a hydrogen atom, R¹⁵ is a hydrogen atom, R¹⁶ is a chlorine atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX434).

The compound (L-1), wherein R¹² is a hydrogen atom, R¹³ is a bromine atom, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX435).

The compound (L-1), wherein R¹² is a hydrogen atom, R¹³ is a hydrogen atom, R¹⁴ is a bromine atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX436).

The compound (L-1), wherein R¹² is a hydrogen atom, R¹³ is a hydrogen atom, R¹⁴ is a hydrogen atom, R¹⁵ is a bromine atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX437).

The compound (L-1), wherein R¹² is a hydrogen atom, R¹³ is a hydrogen atom, R¹⁴ is a hydrogen atom, R¹⁵ is a hydrogen atom, R¹⁶ is a bromine atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX438).

The compound (L-1), wherein R¹² is a hydrogen atom, R¹³ is an iodine atom, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX439).

The compound (L-1), wherein R¹² is a hydrogen atom, R¹³ is a hydrogen atom, R¹⁴ is an iodine atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX440).

The compound (L-1), wherein R¹² is a hydrogen atom, R¹³ is a hydrogen atom, R¹⁴ is a hydrogen atom, R¹⁵ is an iodine atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX441).

The compound (L-1), wherein R¹² is a hydrogen atom, R¹³ is a hydrogen atom, R¹⁴ is a hydrogen atom, R¹⁵ is a hydrogen atom, R¹⁶ is an iodine atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX442).

The compound (L-1), wherein R¹² is a hydrogen atom, R¹³ is a methoxy group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX443).

The compound (L-1), wherein R¹² is a hydrogen atom, R¹³ is a hydrogen atom, R¹⁴ is a methoxy group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX444).

The compound (L-1), wherein R¹² is a hydrogen atom, R¹³ is a hydrogen atom, R¹⁴ is a hydrogen atom, R¹⁵ is a methoxy group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX445).

The compound (L-1), wherein R¹² is a hydrogen atom, R¹³ is a hydrogen atom, R¹⁴ is a hydrogen atom, R¹⁵ is a hydrogen atom, R¹⁶ is a methoxy group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX446).

The compound (L-1), wherein R¹² is a hydrogen atom, R¹³ is a trifluoromethyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX447).

The compound (L-1), wherein R¹² is a hydrogen atom, R¹³ is a hydrogen atom, R¹⁴ is a trifluoromethyl group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX448).

The compound (L-1), wherein R¹² is a hydrogen atom, R¹³ is a hydrogen atom, R¹⁴ is a hydrogen atom, R¹⁵ is a trifluoromethyl group, R¹⁶ is a hydrogen atom, X is NH, and the combination of R¹, R², R³, R⁴, R⁵, R⁶, R⁷, R³, R⁹, R¹⁰, and R¹¹ is any one of the combinations shown in [Table L1] to [Table L13] (hereinafter, referred to as a compound group SX449).

The compound (L-1), wherein R¹² is a hydrogen atom, R¹³ is a hydrogen atom, R¹⁴ is a hydrogen atom, R¹⁵ is a hydrogen atom, R¹⁶ is a trifluoromethyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX450).

The compound (L-1), wherein R¹² is a hydrogen atom, R¹³ is a cyclopropyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX451).

The compound (L-1), wherein R¹² is a hydrogen atom, R¹³ is a hydrogen atom, R¹⁴ is a cyclopropyl group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX452).

The compound (L-1), wherein R¹² is a hydrogen atom, R¹³ is a hydrogen atom, R¹⁴ is a hydrogen atom, R¹⁵ is a cyclopropyl group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX453).

The compound (L-1), wherein R¹² is a hydrogen atom, R¹³ is a hydrogen atom, R¹⁴ is a hydrogen atom, R¹⁵ is a hydrogen atom, R¹⁶ is a cyclopropyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX454).

The compound (L-1), wherein R¹² is a hydrogen atom, R¹³ is a cyclobutyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX455).

The compound (L-1), wherein R¹² is a hydrogen atom, R¹³ is a hydrogen atom, R¹⁴ is a cyclobutyl group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX456).

The compound (L-1), wherein R¹² is a hydrogen atom, R¹³ is a hydrogen atom, R¹⁴ is a hydrogen atom, R¹⁵ is a cyclobutyl group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX457).

The compound (L-1), wherein R¹² is a hydrogen atom, R¹³ is a hydrogen atom, R¹⁴ is a hydrogen atom, R¹⁵ is a hydrogen atom, R¹⁶ is a cyclobutyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX458).

The compound (L-1), wherein R¹² is a hydrogen atom, R¹³ is a cyclopentyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX459).

The compound (1-1), wherein R¹² is a hydrogen atom, R¹³ is a hydrogen atom, R¹⁴ is a cyclopentyl group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX460).

The compound (L-1), wherein R¹² is a hydrogen atom, R¹³ is a hydrogen atom, R¹⁴ is a hydrogen atom, R¹⁵ is a cyclopentyl group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX461).

The compound (L-1), wherein R¹² is a hydrogen atom, R¹³ is a hydrogen atom, R¹⁴ is a hydrogen atom, R¹⁵ is a hydrogen atom, R¹⁶ is a cyclopentyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX462).

The compound (L-1), wherein R¹² is a hydrogen atom, R¹³ is a cyclohexyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX463).

The compound (L-1), wherein R¹² is a hydrogen atom, R¹³ is a hydrogen atom, R¹⁴ is a cyclohexyl group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX464).

The compound (L-1), wherein R¹² is a hydrogen atom, R¹³ is a hydrogen atom, R¹⁴ is a hydrogen atom, R¹⁵ is a cyclohexyl group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX465).

The compound (L-1), wherein R¹² is a hydrogen atom, R¹³ is a hydrogen atom, R¹⁴ is a hydrogen atom, R¹⁵ is a hydrogen atom, R¹⁶ is a cyclohexyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX466).

The compound (L-1), wherein R¹² is a hydrogen atom, R¹³ is a phenyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX467).

The compound (L-1), wherein wherein R¹² is a hydrogen atom, R¹³ is a hydrogen atom, R¹⁴ is a phenyl group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX468).

The compound (L-1), wherein R¹² is a hydrogen atom, R¹³ is a hydrogen atom, R¹⁴ is a hydrogen atom, R¹⁵ is a phenyl group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX469).

The compound (L-1), wherein R¹² is a hydrogen atom, R¹³ is a hydrogen atom, R¹⁴ is a hydrogen atom, R¹⁵ is a hydrogen atom, R¹⁶ is a phenyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX470).

The compound (L-1), wherein R¹² is a hydrogen atom, R¹³ is an acetyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX471).

The compound (L-1), wherein R¹² is a hydrogen atom, R¹³ is a hydrogen atom, R¹⁴ is an acetyl group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX472).

The compound (L-1), wherein R¹² is a hydrogen atom, R¹³ is a hydrogen atom, R¹⁴ is a hydrogen atom, R¹⁵ is an acetyl group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX473).

The compound (L-1), wherein R¹² is a hydrogen atom, R¹³ is a hydrogen atom, R¹⁴ is a hydrogen atom, R¹⁵ is a hydrogen atom, R¹⁶ is an acetyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX474).

The compound (L-1), wherein R¹² is a hydrogen atom, R¹³ is a trifluoroacetyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX475).

The compound (L-1), wherein R¹² is a hydrogen atom, R¹³ is a hydrogen atom, R¹⁴ is a trifluoroacetyl group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX476).

The compound (L-1), wherein R¹² is a hydrogen atom, R¹³ is a hydrogen atom, R¹⁴ is a hydrogen atom, R¹⁵ is a trifluoroacetyl group, R¹⁶ is a hydrogen atom, X is NH, and the combination of R¹, R², R³, R⁴, R⁵, R⁶, R¹, R⁸, R⁹, R¹⁰, and R¹¹ is any one of the combinations shown in [Table L1] to [Table L13] (hereinafter, referred to as a compound group SX477).

The compound (L-1), wherein R¹² is a hydrogen atom, R¹³ is a hydrogen atom, R¹⁴ is a hydrogen atom, R¹⁵ is a hydrogen atom, R¹⁶ is a trifluoroacetyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX478).

The compound (L-1), wherein R¹² is a hydrogen atom, R¹³ is a methoxycarbonyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX479).

The compound (L-1), wherein R¹² is a hydrogen atom, R¹³ is a hydrogen atom, R¹⁴ is a methoxycarbonyl group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX480).

The compound (L-1), wherein R¹² is a hydrogen atom, R¹³ is a hydrogen atom, R¹⁴ is a hydrogen atom, R¹⁵ is a methoxycarbonyl group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX481).

The compound (L-1), wherein R¹² is a hydrogen atom, R¹³ is a hydrogen atom, R¹⁴ is a hydrogen atom, R¹⁵ is a hydrogen atom, R¹⁶ is a methoxycarbonyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX482).

The compound (L-1), wherein R¹² is a hydrogen atom, R¹³ is a trifluoromethoxycarbonyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX483).

The compound (L-1), wherein R¹² is a hydrogen atom, R¹³ is a hydrogen atom, R¹⁴ is a trifluoromethoxycarbonyl group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX484).

The compound (L-1), wherein R¹² is a hydrogen atom, R¹³ is a hydrogen atom, R¹⁴ is a hydrogen atom, R¹⁵ is a trifluoromethoxycarbonyl group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX485).

The compound (L-1), wherein R¹² is a hydrogen atom, R¹³ is a hydrogen atom, R¹⁴ is a hydrogen atom, R¹⁵ is a hydrogen atom, R¹⁶ is a trifluoromethoxycarbonyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX486).

The compound (L-1), wherein R¹² is a hydrogen atom, R¹³ is an acetoxy group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX487).

The compound (L-1), wherein R¹² is a hydrogen atom, R¹³ is a hydrogen atom, R¹⁴ is an acetoxy group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX488).

The compound (L-1), wherein R¹² is a hydrogen atom, R¹³ is a hydrogen atom, R¹⁴ is a hydrogen atom, R¹⁵ is an acetoxy group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX489).

The compound (L-1), wherein R¹² is a hydrogen atom, R¹³ is a hydrogen atom, R¹⁴ is a hydrogen atom, R¹⁵ is a hydrogen atom, R¹⁶ is an acetoxy group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX490).

The compound (L-1), wherein R¹² is a hydrogen atom, R¹³ is a trifluoroacetoxy group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX491).

The compound (L-1), wherein R¹² is a hydrogen atom, R¹³ is a hydrogen atom, R¹⁴ is a trifluoroacetoxy group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX492).

The compound (L-1), wherein R¹² is a hydrogen atom, R¹³ is a hydrogen atom, R¹⁴ is a hydrogen atom, R¹⁵ is a trifluoroacetoxy group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX493).

The compound (L-1), wherein R¹² is a hydrogen atom, R¹³ is a hydrogen atom, R¹⁴ is a hydrogen atom, R¹⁵ is a hydrogen atom, R¹⁶ is a trifluoroacetoxy group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX494).

The compound (L-1), wherein R¹² is a hydrogen atom, R¹³ is a cyano group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX495).

The compound (L-1), wherein R¹² is a hydrogen atom, R¹³ is a hydrogen atom, R¹⁴ is a cyano group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX496).

The compound (L-1), wherein R¹² is a hydrogen atom, R¹³ is a hydrogen atom, R¹⁴ is a hydrogen atom, R¹⁵ is a cyano group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX497).

The compound (L-1), wherein R¹² is a hydrogen atom, R¹³ is a hydrogen atom, R¹⁴ is a hydrogen atom, R¹⁵ is a hydrogen atom, R¹⁶ is a cyano group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX498).

The compound (L-1), wherein R¹² is a hydrogen atom, R¹³ is a formyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group sx499).

The compound (L-1), wherein R¹² is a hydrogen atom, R¹³ is a hydrogen atom, R¹⁴ is a formyl group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX500).

The compound (L-1), wherein R¹² is a hydrogen atom, R¹³ is a hydrogen atom, R¹⁴ is a hydrogen atom, R¹⁵ is a formyl group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX501).

The compound (L-1), wherein R¹² is a hydrogen atom, R¹³ is a hydrogen atom, R¹⁴ is a hydrogen atom, R¹⁵ is a hydrogen atom, Ru6 is a formyl group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX502).

The compound (L-1), wherein R¹² is a hydrogen atom, R¹³ is a carboxy group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX503).

The compound (L-1), wherein R¹² is a hydrogen atom, R¹³ is a hydrogen atom, R¹⁴ is a carboxy group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX504).

The compound (L-1), wherein R¹² is a hydrogen atom, R¹³ is a hydrogen atom, R¹⁴ is a hydrogen atom, R¹⁵ is a carboxy group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX505).

The compound (L-1), wherein R¹² is a hydrogen atom, R¹³ is a hydrogen atom, R¹⁴ is a hydrogen atom, R¹⁵ is a hydrogen atom, R¹⁶ is a carboxy group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX506).

The compound (1-1), wherein R¹² is a hydrogen atom, R¹³ is a nitro group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX507).

The compound (L-1), wherein R¹² is a hydrogen atom, R¹³ is a hydrogen atom, R¹⁴ is a nitro group, R¹⁵ is a hydrogen atom, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX508).

The compound (L-1), wherein R¹² is a hydrogen atom, R¹³ is a hydrogen atom, R¹⁴ is a hydrogen atom, R¹⁵ is a nitro group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX509).

The compound (L-1), wherein R¹² is a hydrogen atom, R¹³ is a hydrogen atom, R¹⁴ is a hydrogen atom, R¹⁵ is a hydrogen atom, R¹⁶ is a nitro group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX510).

The compound (L-1), wherein R¹² is a hydrogen atom, R¹³ is a hydroxy group, R¹⁴ is a hydrogen atom, R¹⁵ is a hydrogen atom, R¹⁶ is a hydroxy group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX511).

The compound (L-1), wherein R¹² is a hydrogen atom, R¹³ is a hydrogen atom, R¹⁴ is a hydroxy group, R¹⁵ is a hydrogen atom, R¹⁶ is a hydroxy group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX512).

The compound (L-1), wherein R¹² is a hydrogen atom, R¹³ is a hydrogen atom, R¹⁴ is a hydrogen atom, R¹⁵ is a hydroxy group, R¹⁶ is a hydrogen atom, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX513).

The compound (L-1), wherein R¹² is a hydrogen atom, R¹³ is a hydrogen atom, R¹⁴ is a hydrogen atom, R¹⁵ is a hydrogen atom, R¹⁶ is a hydroxy group, X is NH, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX514).

The compound (L-1), wherein R¹² is a hydrogen atom, R¹³ is a hydrogen atom, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX515).

The compound (L-1), wherein R¹² is a methyl group, R¹³ is a hydrogen atom, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX516).

The compound (L-1), wherein R¹² is a hydrogen atom, R¹³ is a methyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX517).

The compound (L-1), wherein R¹² is a hydrogen atom, R¹³ is a hydrogen atom, R¹⁴ is a methyl group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX518).

The compound (L-1), wherein R¹² is a hydrogen atom, R¹³ is a hydrogen atom, R¹⁴ is a hydrogen atom, R¹⁵ is a methyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX519).

The compound (L-1), wherein R¹² is a hydrogen atom, R¹³ is a hydrogen atom, R¹⁴ is a hydrogen atom, R¹⁵ is a hydrogen atom, R¹⁶ is a methyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX520).

The compound (L-1), wherein R¹² is a hydrogen atom, R¹³ is a methyl group, R¹⁴ is a methyl group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX521).

The compound (L-1), wherein R¹² is a hydrogen atom, R¹³ is a methyl group, R¹⁴ is a hydrogen atom, R¹⁵ is a methyl group, R¹⁶ is a hydrogen atom, X is NCH₃, and the combination of R¹, R², R³, R⁴, R⁵, R⁶, R⁷, R⁸, R⁹, R¹⁰, and R¹¹. is any one of the combinations shown in [Table L1] to [Table L13] (hereinafter, referred to as a compound group SX522).

The compound (L-1), wherein R¹² is a hydrogen atom, R¹³ is a methyl group, R¹⁴ is a hydrogen atom, R¹⁵ is a hydrogen atom, R¹⁶ is a methyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX523).

The compound (L-1), wherein R¹² is a hydrogen atom, R¹³ is a hydrogen atom, R¹⁴ is a methyl group, R¹⁵ is a methyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX524).

The compound (L-1), wherein R¹² is a hydrogen atom, R¹³ is a hydrogen atom, R¹⁴ is a methyl group, R¹⁵ is a hydrogen atom, R¹⁶ is a methyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX525).

The compound (L-1), wherein R¹² is a hydrogen atom, R¹³ is a hydrogen atom, R¹⁴ is a hydrogen atom, R¹⁵ is a methyl group, R¹⁶ is a methyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX526).

The compound (L-1), wherein R¹² is a hydrogen atom, R¹³ is a methyl group, R¹⁴ is a methyl group, R¹⁵ is a methyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX527).

The compound (L-1), wherein R¹² is a hydrogen atom, R¹³ is a hydrogen atom, R¹⁴ is a methyl group, R¹⁵ is a methyl group, R¹⁶ is a methyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX528).

The compound (L-1), wherein R¹² is a hydrogen atom, R¹³ is a fluorine atom, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX529).

The compound (L-1), wherein R¹² is a hydrogen atom, R¹³ is a hydrogen atom, R¹⁴ is a fluorine atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX530).

The compound (L-1), wherein R¹² is a hydrogen atom, R¹³ is a hydrogen atom, R¹⁴ is a hydrogen atom, R¹⁵ is a fluorine atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX531).

The compound (L-1), wherein R¹² is a hydrogen atom, R¹³ is a hydrogen atom, R¹⁴ is a hydrogen atom, R¹⁵ is a hydrogen atom, R¹⁶ is a fluorine atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX532).

The compound (L-1), wherein R¹² is a hydrogen atom, R¹³ is a chlorine atom, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX533).

The compound (L-1), wherein R¹² is a hydrogen atom, R¹³ is a hydrogen atom, R¹⁴ is a chlorine atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX534).

The compound (L-1), wherein R¹² is a hydrogen atom, R¹³ is a hydrogen atom, R¹⁴ is a hydrogen atom, R¹⁵ is a chlorine atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX535).

The compound (L-1), wherein R¹² is a hydrogen atom, R¹³ is a hydrogen atom, R¹⁴ is a hydrogen atom, R¹⁵ is a hydrogen atom, R¹⁶ is a chlorine atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX536).

The compound (L-1), wherein R¹² is a hydrogen atom, R¹³ is a bromine atom, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX537).

The compound (L-1), wherein R¹² is a hydrogen atom, R¹³ is a hydrogen atom, R¹⁴ is a bromine atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX538).

The compound (L-1), wherein R¹² is a hydrogen atom, R¹³ is a hydrogen atom, R¹⁴ is a hydrogen atom, R¹⁵ is a bromine atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX539).

The compound (L-1), wherein R¹² is a hydrogen atom, R¹³ is a hydrogen atom, R¹⁴ is a hydrogen atom, R¹⁵ is a hydrogen atom, R¹⁶ is a bromine atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX540).

The compound (L-1), wherein R¹² is a hydrogen atom, R¹³ is an iodine atom, R¹⁴ is a hydrogen atom, R¹⁵ is a hydrogen atom, R¹⁶ is a bromine atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX541).

The compound (L-1), wherein R¹² is a hydrogen atom, R¹³ is a hydrogen atom, R¹⁴ is an iodine atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX542).

The compound (L-1), wherein R¹² is a hydrogen atom, R¹³ is a hydrogen atom, R¹⁴ is a hydrogen atom, R¹⁵ is an iodine atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX543).

The compound (L-1), wherein R¹² is a hydrogen atom, R¹³ is a hydrogen atom, R¹⁴ is a hydrogen atom, R¹⁵ is a hydrogen atom, R¹⁶ is an iodine atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX544).

The compound (L-1), wherein R¹² is a hydrogen atom, R¹³ is a methoxy group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX545).

The compound (L-1), wherein R¹² is a hydrogen atom, R¹³ is a hydrogen atom, R¹⁴ is a methoxy group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX546).

The compound (L-1), wherein R¹² is a hydrogen atom, R¹³ is a hydrogen atom, R¹⁴ is a hydrogen atom, R¹⁵ is a methoxy group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX547).

The compound (L-1), wherein R¹² is a hydrogen atom, R¹³ is a hydrogen atom, R¹⁴ is a hydrogen atom, R¹⁵ is a hydrogen atom, R¹⁶ is a methoxy group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX548).

The compound (L-1), wherein R¹² is a hydrogen atom, R¹³ is a trifluoromethyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX549).

The compound (L-1), wherein R¹² is a hydrogen atom, R¹³ is a hydrogen atom, R¹⁴ is a trifluoromethyl group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX550).

The compound (L-1), wherein R¹² is a hydrogen atom, R¹³ is a hydrogen atom, R¹⁴ is a hydrogen atom, R¹⁵ is a trifluoromethyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX551).

The compound (L-1), wherein R¹² is a hydrogen atom, R¹³ is a hydrogen atom, R¹⁴ is a hydrogen atom, R¹⁵ is a hydrogen atom, R¹⁶ is a trifluoromethyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX552).

The compound (L-1), wherein R¹² is a hydrogen atom, R¹³ is a cyclopropyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX553).

The compound (L-1), wherein R¹² is a hydrogen atom, R¹³ is a hydrogen atom, R¹⁴ is a cyclopropyl group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX554).

The compound (L-1), wherein R¹² is a hydrogen atom, R¹³ is a hydrogen atom, R¹⁴ is a hydrogen atom, R¹⁵ is a cyclopropyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX555).

The compound (L-1), wherein R¹² is a hydrogen atom, R¹³ is a hydrogen atom, R¹⁴ is a hydrogen atom, R¹⁵ is a hydrogen atom, R¹⁶ is a cyclopropyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX556).

The compound (L-1), wherein R¹² is a hydrogen atom, R¹³ is a cyclobutyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX557).

The compound (L-1), wherein R¹² is a hydrogen atom, R¹³ is a hydrogen atom, R¹⁴ is a cyclobutyl group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX558).

The compound (L-1), wherein R¹² is a hydrogen atom, R¹³ is a hydrogen atom, R¹⁴ is a hydrogen atom, R¹⁵ is a cyclobutyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX559).

The compound (L-1), wherein R¹² is a hydrogen atom, R¹³ is a hydrogen atom, R¹⁴ is a hydrogen atom, R¹⁵ is a hydrogen atom, R¹⁶ is a cyclobutyl group, X is NCH₃, and the combination of R¹, R², R³, R⁴, R⁵, R⁶, R⁷, R⁸, R⁹, R¹⁰, and R¹¹. is any one of the combinations shown in [Table L1] to [Table L13] (hereinafter, referred to as a compound group SX560).

The compound (L-1), wherein R¹² is a hydrogen atom, R¹³ is a cyclopentyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX561).

The compound (L-1), wherein R¹² is a hydrogen atom, R¹³ is a hydrogen atom, R¹⁴ is a cyclopentyl group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX562).

The compound (L-1), wherein R¹² is a hydrogen atom, R¹³ is a hydrogen atom, R¹⁴ is a hydrogen atom, R¹⁵ is a cyclopentyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX563).

The compound (L-1), wherein R¹² is a hydrogen atom, R¹³ is a hydrogen atom, R¹⁴ is a hydrogen atom, R¹⁵ is a hydrogen atom, R¹⁶ is a cyclopentyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX564).

The compound (L-1), wherein R¹² is a hydrogen atom, R¹³ is a cyclohexyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX565).

The compound (L-1), wherein R¹² is a hydrogen atom, R¹³ is a hydrogen atom, R¹⁴ is a cyclohexyl group, R¹⁵ is a hydrogen atom, R¹⁶ is a bromine atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX566).

The compound (L-1), wherein R¹² is a hydrogen atom, R¹³ is a hydrogen atom, R¹⁴ is a hydrogen atom, R¹⁵ is a cyclohexyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX567).

The compound (L-1), wherein R¹² is a hydrogen atom, R¹³ is a hydrogen atom, R¹⁴ is a hydrogen atom, R¹⁵ is a hydrogen atom, R¹⁶ is a cyclohexyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX568).

The compound (L-1), wherein R¹² is a hydrogen atom, R¹³ is a phenyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX569).

The compound (L-1), wherein R¹² is a hydrogen atom, R¹³ is a hydrogen atom, R¹⁴ is a phenyl group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX570).

The compound (L-1), wherein R¹² is a hydrogen atom, R¹³ is a hydrogen atom, R¹⁴ is a hydrogen atom, R¹⁵ is a phenyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX571).

The compound (L-1), wherein R¹² is a hydrogen atom, R¹³ is a hydrogen atom, R¹⁴ is a hydrogen atom, R¹⁵ is a hydrogen atom, R¹⁶ is a phenyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX572).

The compound (L-1), wherein R¹² is a hydrogen atom, R¹³ is an acetyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX573).

The compound (L-1), wherein R¹² is a hydrogen atom, R¹³ is a hydrogen atom, R¹⁴ is an acetyl group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX574).

The compound (L-1), wherein R¹² is a hydrogen atom, R¹³ is a hydrogen atom, R¹⁴ is a hydrogen atom, R¹⁵ is an acetyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX575).

The compound (L-1), wherein R¹² is a hydrogen atom, R¹³ is a hydrogen atom, R¹⁴ is a hydrogen atom, R¹⁵ is a hydrogen atom, R¹⁶ is an acetyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX576).

The compound (L-1), wherein R¹² is a hydrogen atom, R¹³ is a trifluoroacetyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX577).

The compound (L-1), wherein R¹² is a hydrogen atom, R¹³ is a hydrogen atom, R¹⁴ is a trifluoroacetyl group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX578).

The compound (L-1), wherein R¹² is a hydrogen atom, R¹³ is a hydrogen atom, R¹⁴ is a hydrogen atom, R¹⁵ is a trifluoroacetyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX579).

The compound (L-1), wherein R¹² is a hydrogen atom, R¹³ is a hydrogen atom, R¹⁴ is a hydrogen atom, R¹⁵ is a hydrogen atom, R¹⁶ is a trifluoroacetyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX580).

The compound (L-1), wherein R¹² is a hydrogen atom, R¹³ is a methoxycarbonyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX581).

The compound (L-1), wherein R¹² is a hydrogen atom, R¹³ is a hydrogen atom, R¹⁴ is a methoxycarbonyl group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX582).

The compound (L-1), wherein R¹² is a hydrogen atom, R¹³ is a hydrogen atom, R¹⁴ is a hydrogen atom, R¹⁵ is a methoxycarbonyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX583).

The compound (L-1), wherein R¹² is a hydrogen atom, R¹³ is a hydrogen atom, R¹⁴ is a hydrogen atom, R¹⁵ is a hydrogen atom, R¹⁶ is a methoxycarbonyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX584).

The compound (L-1), wherein R¹² is a hydrogen atom, R¹³ is a trifluoromethoxycarbonyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX585).

The compound (L-1), wherein R¹² is a hydrogen atom, R¹³ is a hydrogen atom, R¹⁴ is a trifluoromethoxycarbonyl group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX586).

The compound (L-1), wherein R¹² is a hydrogen atom, R¹³ is a hydrogen atom, R¹⁴ is a hydrogen atom, R¹⁵ is a trifluoromethoxycarbonyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX587).

The compound (L-1), wherein R¹² is a hydrogen atom, R¹³ is a hydrogen atom, R¹⁴ is a hydrogen atom, R¹⁵ is a hydrogen atom, R¹⁶ is a trifluoromethoxycarbonyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX588).

The compound (L-1), wherein R¹² is a hydrogen atom, R¹³ is an acetoxy group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX589).

The compound (L-1), wherein R¹² is a hydrogen atom, R¹³ is a hydrogen atom, R¹⁴ is an acetoxy group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX590).

The compound (L-1), wherein R¹² is a hydrogen atom, R¹³ is a hydrogen atom, R¹⁴ is a hydrogen atom, R¹⁵ is an acetoxy group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX591).

The compound (L-1), wherein R¹² is a hydrogen atom, R¹³ is a hydrogen atom, R¹⁴ is a hydrogen atom, R¹⁵ is a hydrogen atom, R¹⁶ is an acetoxy group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX592).

The compound (L-1), wherein R¹² is a hydrogen atom, R¹³ is a trifluoroacetoxy group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX593).

The compound (L-1), wherein R¹² is a hydrogen atom, R¹³ is a hydrogen atom, R¹⁴ is a trifluoroacetoxy group, RE5 is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX594).

The compound (L-1), wherein R¹² is a hydrogen atom, R¹³ is a hydrogen atom, R¹⁴ is a hydrogen atom, R¹⁵ is a trifluoroacetoxy group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX595).

The compound (L-1), wherein R¹² is a hydrogen atom, R¹³ is a hydrogen atom, R¹⁴ is a hydrogen atom, R¹⁵ is a hydrogen atom, R¹⁶ is a trifluoroacetoxy group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX596).

The compound (L-1), wherein R¹² is a hydrogen atom, R¹³ is a cyano group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁶, R⁹, R¹⁰, and R¹¹. is any one of the combinations shown in [Table L1] to [Table L13] (hereinafter, referred to as a compound group SX597).

The compound (L-1), wherein R¹² is a hydrogen atom, R¹³ is a hydrogen atom, R¹⁴ is a cyano group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX598).

The compound (L-1), wherein R¹² is a hydrogen atom, R¹³ is a hydrogen atom, R¹⁴ is a hydrogen atom, R¹⁵ is a cyano group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX599).

The compound (L-1), wherein R¹² is a hydrogen atom, R¹³ is a hydrogen atom, R¹⁴ is a hydrogen atom, R¹⁵ is a hydrogen atom, R¹⁶ is a cyano group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX600).

The compound (L-1), wherein R¹² is a hydrogen atom, R¹³ is a formyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX601).

The compound (L-1), wherein R¹² is a hydrogen atom, R¹³ is a hydrogen atom, R¹⁴ is a formyl group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX602).

The compound (L-1), wherein R¹² is a hydrogen atom, R¹³ is a hydrogen atom, R¹⁴ is a hydrogen atom, R¹⁵ is a formyl group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX603).

The compound (L-1), wherein R¹² is a hydrogen atom, R¹³ is a hydrogen atom, R¹⁴ is a hydrogen atom, R¹⁵ is a hydrogen atom, R¹⁶ is a formyl group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX604).

The compound (L-1), wherein R¹² is a hydrogen atom, R¹³ is a carboxy group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX605).

The compound (L-1), wherein R¹² is a hydrogen atom, R¹³ is a hydrogen atom, R¹⁴ is a carboxy group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX606).

The compound (L-1), wherein R¹² is a hydrogen atom, R¹³ is a hydrogen atom, R¹⁴ is a hydrogen atom, R¹⁵ is a carboxy group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX607).

The compound (L-1), wherein R¹² is a hydrogen atom, R¹³ is a hydrogen atom, R¹⁴ is a hydrogen atom, R¹⁵ is a hydrogen atom, R¹⁶ is a carboxy group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX608).

The compound (L-1), wherein R¹² is a hydrogen atom, R¹³ is a nitro group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX609).

The compound (L-1), wherein R¹² is a hydrogen atom, R¹³ is a hydrogen atom, R¹⁴ is a nitro group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX610).

The compound (L-1), wherein R¹² is a hydrogen atom, R¹³ is a hydrogen atom, R¹⁴ is a hydrogen atom, R¹⁵ is a nitro group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX611).

The compound (L-1), wherein R¹² is a hydrogen atom, R¹³ is a hydrogen atom, R¹⁴ is a hydrogen atom, R¹⁵ is a hydrogen atom, R¹⁶ is a nitro group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX612).

The compound (L-1), wherein R¹² is a hydrogen atom, R¹³ is a hydroxy group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX613).

The compound (L-1), wherein R¹² is a hydrogen atom, R¹³ is a hydrogen atom, R¹⁴ is a hydroxy group, R¹⁵ is a hydrogen atom, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX614).

The compound (L-1), wherein R¹² is a hydrogen atom, R¹³ is a hydrogen atom, R¹⁴ is a hydrogen atom, R¹⁵ is a hydroxy group, R¹⁶ is a hydrogen atom, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX615).

The compound (L-1), wherein R¹² is a hydrogen atom, R¹³ is a hydrogen atom, R¹⁴ is a hydrogen atom, R¹⁵ is a hydrogen atom, R¹⁶ is a hydroxy group, X is NCH₃, and the combination of R¹, R², R³, R⁴, R⁵, R⁶, R⁷, R⁸, R⁹, R¹⁰, and R¹¹ is any one of the combinations shown in [Table L1] to [Table L13] (hereinafter, referred to as a compound group SX616).

A compound represented by formula (L-2) (hereinafter, referred to as a compound (L-2)),

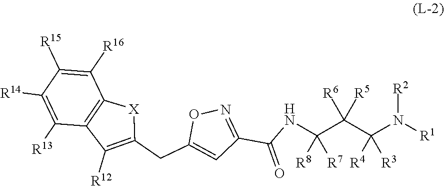

wherein R¹² is a hydrogen atom, R¹³ is a hydrogen atom, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L14] to [Table L99] (hereinafter, referred to as a compound group SX617).

TABLE L14

| R¹ | R² | R³ | R⁴ | R⁵ | R⁶ | R⁷ | R⁸ |
|---|---|---|---|---|---|---|---|
| Me | Me | Et | Et | H | H | H | H |
| Me | Me | H | H | Et | Et | H | H |
| Me | Me | H | H | H | H | Et | Et |
| Et | Me | Et | Et | H | H | H | H |
| Et | Me | H | H | Et | Et | H | H |
| Et | Me | H | H | H | H | Et | Et |
| Et | Et | Et | Et | H | H | H | H |
| Et | Et | H | H | Et | Et | H | H |
| Et | Et | H | H | H | H | Et | Et |
| i-Pr | Me | Et | Et | H | H | H | H |
| i-Pr | Me | H | H | Et | Et | H | H |
| i-Pr | Me | H | H | H | H | Et | Et |
| i-Pr | Et | Et | Et | H | H | H | H |
| i-Pr | Et | H | H | Et | Et | H | H |
| i-Pr | Et | H | H | H | H | Et | Et |
| Pr | Me | Et | Et | H | H | H | H |
| Pr | Me | H | H | Et | Et | H | H |
| Pr | Me | H | H | H | H | Et | Et |
| Pr | Et | Et | Et | H | H | H | H |
| Pr | Et | H | H | Et | Et | H | H |
| Pr | Et | H | H | H | H | Et | Et |
| Pr | i-Pr | Et | Et | H | H | H | H |
| Pr | i-Pr | H | H | Et | Et | H | H |
| Pr | i-Pr | H | H | H | H | Et | Et |
| Pr | Pr | Et | Et | H | H | H | H |
| Pr | Pr | H | H | Et | Et | H | H |
| Pr | Pr | H | H | H | H | Et | Et |

TABLE L15

| R¹ | R² | R³ | R⁴ | R⁵ | R⁶ | R⁷ | R⁸ |
|---|---|---|---|---|---|---|---|
| Bu | Me | Et | Et | H | H | H | H |
| Bu | Me | H | H | Et | Et | H | H |
| Bu | Me | H | H | H | H | Et | Et |
| Bu | Et | Et | Et | H | H | H | H |
| Bu | Et | H | H | Et | Et | H | H |
| Bu | Et | H | H | H | H | Et | Et |
| Bu | i-Pr | Et | Et | H | H | H | H |
| Bu | i-Pr | H | H | Et | Et | H | H |
| Bu | i-Pr | H | H | H | H | Et | Et |
| Bu | Pr | Et | Et | H | H | H | H |
| Bu | Pr | H | H | Et | Et | H | H |
| Bu | Pr | H | H | H | H | Et | Et |
| Bu | Bu | Et | Et | H | H | H | H |
| Bu | Bu | H | H | Et | Et | H | H |
| Bu | Bu | H | H | H | H | Et | Et |
| t-Bu | Me | Et | Et | H | H | H | H |
| t-Bu | Me | H | H | Et | Et | H | H |
| t-Bu | Me | H | H | H | H | Et | Et |
| t-Bu | Et | Et | Et | H | H | H | H |
| t-Bu | Et | H | H | Et | Et | H | H |
| t-Bu | Et | H | H | H | H | Et | Et |
| t-Bu | Pr | Et | Et | H | H | H | H |
| t-Bu | Pr | H | H | Et | Et | H | H |
| t-Bu | Pr | H | H | H | H | Et | Et |
| t-Bu | Bu | Et | Et | H | H | H | H |
| t-Bu | Bu | H | H | Et | Et | H | H |
| t-Bu | Bu | H | H | H | H | Et | Et |

TABLE L16

| R¹ | R² | R³ | R⁴ | R⁵ | R⁶ | R⁷ | R⁸ |
|---|---|---|---|---|---|---|---|
| Me | Me | i-Pr | H | H | H | H | H |
| Me | Me | H | H | i-Pr | H | H | H |
| Me | Me | H | H | H | H | i-Pr | H |
| Et | Me | i-Pr | H | H | H | H | H |
| Et | Me | H | H | i-Pr | H | H | H |
| Et | Me | H | H | H | H | i-Pr | H |
| Et | Et | i-Pr | H | H | H | H | H |
| Et | Et | H | H | i-Pr | H | H | H |
| Et | Et | H | H | H | H | i-Pr | H |
| i-Pr | Me | i-Pr | H | H | H | H | H |
| i-Pr | Me | H | H | i-Pr | H | H | H |
| i-Pr | Me | H | H | H | H | i-Pr | H |
| i-Pr | Et | i-Pr | H | H | H | H | H |
| i-Pr | Et | H | H | i-Pr | H | H | H |
| i-Pr | Et | H | H | H | H | i-Pr | H |
| Pr | Me | i-Pr | H | H | H | H | H |
| Pr | Me | H | H | i-Pr | H | H | H |
| Pr | Me | H | H | H | H | i-Pr | H |
| Pr | Et | i-Pr | H | H | H | H | H |
| Pr | Et | H | H | i-Pr | H | H | H |
| Pr | Et | H | H | H | H | i-Pr | H |
| Pr | i-Pr | i-Pr | H | H | H | H | H |
| Pr | i-Pr | H | H | i-Pr | H | H | H |
| Pr | i-Pr | H | H | H | H | i-Pr | H |
| Pr | Pr | i-Pr | H | H | H | H | H |
| Pr | Pr | H | H | i-Pr | H | H | H |
| Pr | Pr | H | H | H | H | i-Pr | H |

TABLE L17

| R¹ | R² | R³ | R⁴ | R⁵ | R⁶ | R⁷ | R⁸ |
|---|---|---|---|---|---|---|---|
| Bu | Me | i-Pr | H | H | H | H | H |
| Bu | Me | H | H | i-Pr | H | H | H |
| Bu | Me | H | H | H | H | i-Pr | H |
| Bu | Et | i-Pr | H | H | H | H | H |
| Bu | Et | H | H | i-Pr | H | H | H |
| Bu | Et | H | H | H | H | i-Pr | H |
| Bu | i-Pr | i-Pr | H | H | H | H | H |
| Bu | i-Pr | H | H | i-Pr | H | H | H |
| Bu | i-Pr | H | H | H | H | i-Pr | H |
| Bu | Pr | i-Pr | H | H | H | H | H |
| Bu | Pr | H | H | i-Pr | H | H | H |
| Bu | Pr | H | H | H | H | i-Pr | H |
| Bu | Bu | i-Pr | H | H | H | H | H |
| Bu | Bu | H | H | i-Pr | H | H | H |
| Bu | Bu | H | H | H | H | i-Pr | H |
| t-Bu | Me | i-Pr | H | H | H | H | H |
| t-Bu | Me | H | H | i-Pr | H | H | H |
| t-Bu | Me | H | H | H | H | i-Pr | H |
| t-Bu | Et | i-Pr | H | H | H | H | H |
| t-Bu | Et | H | H | i-Pr | H | H | H |
| t-Bu | Et | H | H | H | H | i-Pr | H |
| t-Bu | Pr | i-Pr | H | H | H | H | H |
| t-Bu | Pr | H | H | i-Pr | H | H | H |
| t-Bu | Pr | H | H | H | H | i-Pr | H |
| t-Bu | Bu | i-Pr | H | H | H | H | H |
| t-Bu | Bu | H | H | i-Pr | H | H | H |
| t-Bu | Bu | H | H | H | H | i-Pr | H |

TABLE L18

| R¹ | R² | R³ | R⁴ | R⁵ | R⁶ | R⁷ | R⁸ |
|---|---|---|---|---|---|---|---|
| Me | Me | Pr | Pr | H | H | H | H |
| Me | Me | H | H | Pr | Pr | H | H |
| Me | Me | H | H | H | H | Pr | Pr |
| Et | Me | Pr | Pr | H | H | H | H |
| Et | Me | H | H | Pr | Pr | H | H |
| Et | Me | H | H | H | H | Pr | Pr |
| Et | Et | Pr | Pr | H | H | H | H |
| Et | Et | H | H | Pr | Pr | H | H |
| Et | Et | H | H | H | H | Pr | Pr |
| i-Pr | Me | Pr | Pr | H | H | H | H |
| i-Pr | Me | H | H | Pr | Pr | H | H |
| i-Pr | Me | H | H | H | H | Pr | Pr |
| i-Pr | Et | Pr | Pr | H | H | H | H |
| i-Pr | Et | H | H | Pr | Pr | H | H |
| i-Pr | Et | H | H | H | H | Pr | Pr |
| Pr | Me | Pr | Pr | H | H | H | H |
| Pr | Me | H | H | Pr | Pr | H | H |
| Pr | Me | H | H | H | H | Pr | Pr |
| Pr | Et | Pr | Pr | H | H | H | H |
| Pr | Et | H | H | Pr | Pr | H | H |
| Pr | Et | H | H | H | H | Pr | Pr |
| Pr | i-Pr | Pr | Pr | H | H | H | H |
| Pr | i-Pr | H | H | Pr | Pr | H | H |
| Pr | i-Pr | H | H | H | H | Pr | Pr |
| Pr | Pr | Pr | Pr | H | H | H | H |
| Pr | Pr | H | H | Pr | Pr | H | H |
| Pr | Pr | H | H | H | H | Pr | Pr |

TABLE L19

| R¹ | R² | R³ | R⁴ | R⁵ | R⁶ | R⁷ | R⁸ |
|---|---|---|---|---|---|---|---|
| Bu | Me | Pr | Pr | H | H | H | H |
| Bu | Me | H | H | Pr | Pr | H | H |
| Bu | Me | H | H | H | H | Pr | Pr |
| Bu | Et | Pr | Pr | H | H | H | H |
| Bu | Et | H | H | Pr | Pr | H | H |
| Bu | Et | H | H | H | H | Pr | Pr |
| Bu | i-Pr | Pr | Pr | H | H | H | H |
| Bu | i-Pr | H | H | Pr | Pr | H | H |
| Bu | i-Pr | H | H | H | H | Pr | Pr |
| Bu | Pr | Pr | Pr | H | H | H | H |
| Bu | Pr | H | H | Pr | Pr | H | H |
| Bu | Pr | H | H | H | H | Pr | Pr |
| Bu | Bu | Pr | Pr | H | H | H | H |
| Bu | Bu | H | H | Pr | Pr | H | H |
| Bu | Bu | H | H | H | H | Pr | Pr |
| t-Bu | Me | Pr | Pr | H | H | H | H |
| t-Bu | Me | H | H | Pr | Pr | H | H |
| t-Bu | Me | H | H | H | H | Pr | Pr |
| t-Bu | Et | Pr | Pr | H | H | H | H |
| t-Bu | Et | H | H | Pr | Pr | H | H |
| t-Bu | Et | H | H | H | H | Pr | Pr |
| t-Bu | Pr | Pr | Pr | H | H | H | H |
| t-Bu | Pr | H | H | Pr | Pr | H | H |
| t-Bu | Pr | H | H | H | H | Pr | Pr |
| t-Bu | Bu | Pr | Pr | H | H | H | H |
| t-Bu | Bu | H | H | Pr | Pr | H | H |
| t-Bu | Bu | H | H | H | H | Pr | Pr |

TABLE L20

| R¹ | R² | R³ | R⁴ | R⁵ | R⁶ | R⁷ | R⁸ |
|---|---|---|---|---|---|---|---|
| Me | Me | Bu | Bu | H | H | H | H |
| Me | Me | H | H | Bu | Bu | H | H |
| Me | Me | H | H | H | H | Bu | Bu |
| Et | Me | Bu | Bu | H | H | H | H |
| Et | Me | H | H | Bu | Bu | H | H |
| Et | Me | H | H | H | H | Bu | Bu |
| Et | Et | Bu | Bu | H | H | H | H |
| Et | Et | H | H | Bu | Bu | H | H |
| Et | Et | H | H | H | H | Bu | Bu |
| i-Pr | Me | Bu | Bu | H | H | H | H |
| i-Pr | Me | H | H | Bu | Bu | H | H |
| i-Pr | Me | H | H | H | H | Bu | Bu |
| i-Pr | Et | Bu | Bu | H | H | H | H |
| i-Pr | Et | H | H | Bu | Bu | H | H |
| i-Pr | Et | H | H | H | H | Bu | Bu |
| Pr | Me | Bu | Bu | H | H | H | H |
| Pr | Me | H | H | Bu | Bu | H | H |
| Pr | Me | H | H | H | H | Bu | Bu |
| Pr | Et | Bu | Bu | H | H | H | H |
| Pr | Et | H | H | Bu | Bu | H | H |
| Pr | Et | H | H | H | H | Bu | Bu |
| Pr | i-Pr | Bu | Bu | H | H | H | H |
| Pr | i-Pr | H | H | Bu | Bu | H | H |
| Pr | i-Pr | H | H | H | H | Bu | Bu |
| Pr | Pr | Bu | Bu | H | H | H | H |
| Pr | Pr | H | H | Bu | Bu | H | H |
| Pr | Pr | H | H | H | H | Bu | Bu |

TABLE L21

| R¹ | R² | R³ | R⁴ | R⁵ | R⁶ | R⁷ | R⁸ |
|---|---|---|---|---|---|---|---|
| Bu | Me | Bu | Bu | H | H | H | H |
| Bu | Me | H | H | Bu | Bu | H | H |
| Bu | Me | H | H | H | H | Bu | Bu |
| Bu | Et | Bu | Bu | H | H | H | H |
| Bu | Et | H | H | Bu | Bu | H | H |
| Bu | Et | H | H | H | H | Bu | Bu |
| Bu | i-Pr | Bu | Bu | H | H | H | H |
| Bu | i-Pr | H | H | Bu | Bu | H | H |
| Bu | i-Pr | H | H | H | H | Bu | Bu |
| Bu | Pr | Bu | Bu | H | H | H | H |
| Bu | Pr | H | H | Bu | Bu | H | H |
| Bu | Pr | H | H | H | H | Bu | Bu |
| Bu | Bu | Bu | Bu | H | H | H | H |
| Bu | Bu | H | H | Bu | Bu | H | H |
| Bu | Bu | H | H | H | H | Bu | Bu |
| t-Bu | Me | Bu | Bu | H | H | H | H |
| t-Bu | Me | H | H | Bu | Bu | H | H |
| t-Bu | Me | H | H | H | H | Bu | Bu |
| t-Bu | Et | Bu | Bu | H | H | H | H |
| t-Bu | Et | H | H | Bu | Bu | H | H |
| t-Bu | Et | H | H | H | H | Bu | Bu |
| t-Bu | Pr | Bu | Bu | H | H | H | H |
| t-Bu | Pr | H | H | Bu | Bu | H | H |
| t-Bu | Pr | H | H | H | H | Bu | Bu |
| t-Bu | Bu | Bu | Bu | H | H | H | H |
| t-Bu | Bu | H | H | Bu | Bu | H | H |
| t-Bu | Bu | H | H | H | H | Bu | Bu |

TABLE L22

| | | | | | | | |
|---|---|---|---|---|---|---|---|
| R¹ | R² | R³ | R⁴ | R⁵ | R⁶ | R⁷ | R⁸ |

| | | | | | | |
|---|---|---|---|---|---|---|
| H | H | —(CH₂)₂— | H | H | H | H |

| | | | | | | |
|---|---|---|---|---|---|---|
| H | H | H | H | —(CH₂)₂— | H | H |

| | | | | | | |
|---|---|---|---|---|---|---|
| H | H | H | H | H | H | —(CH₂)₂— |

| | | | | | | |
|---|---|---|---|---|---|---|
| Me | H | —(CH₂)₂— | H | H | H | H |

| | | | | | | |
|---|---|---|---|---|---|---|
| Me | H | H | H | —(CH₂)₂— | H | H |

| | | | | | | |
|---|---|---|---|---|---|---|
| Me | H | H | H | H | H | —(CH₂)₂— |

| | | | | | | |
|---|---|---|---|---|---|---|
| Me | Me | —(CH₂)₂— | H | H | H | H |

| | | | | | | |
|---|---|---|---|---|---|---|
| Me | Me | H | H | —(CH₂)₂— | H | H |

| | | | | | | |
|---|---|---|---|---|---|---|
| Me | Me | H | H | H | H | —(CH₂)₂— |

| | | | | | | |
|---|---|---|---|---|---|---|
| Et | H | —(CH₂)₂— | H | H | H | H |

| | | | | | | |
|---|---|---|---|---|---|---|
| Et | H | H | H | —(CH₂)₂— | H | H |

| | | | | | | |
|---|---|---|---|---|---|---|
| Et | H | H | H | H | H | —(CH₂)₂— |

| | | | | | | |
|---|---|---|---|---|---|---|
| Et | Me | —(CH₂)₂— | H | H | H | H |

| | | | | | | |
|---|---|---|---|---|---|---|
| Et | Me | H | H | —(CH₂)₂— | H | H |

| | | | | | | |
|---|---|---|---|---|---|---|
| Et | Me | H | H | H | H | —(CH₂)₂— |

| | | | | | | |
|---|---|---|---|---|---|---|
| Et | Et | —(CH₂)₂— | H | H | H | H |

| | | | | | | |
|---|---|---|---|---|---|---|
| Et | Et | H | H | —(CH₂)₂— | H | H |

| | | | | | | |
|---|---|---|---|---|---|---|
| Et | Et | H | H | H | H | —(CH₂)₂— |

| | | | | | | |
|---|---|---|---|---|---|---|
| i-Pr | H | —(CH₂)₂— | H | H | H | H |

| | | | | | | |
|---|---|---|---|---|---|---|
| i-Pr | H | H | H | —(CH₂)₂— | H | H |

| | | | | | | |
|---|---|---|---|---|---|---|
| i-Pr | H | H | H | H | H | —(CH₂)₂— |

| | | | | | | |
|---|---|---|---|---|---|---|
| i-Pr | Me | —(CH₂)₂— | H | H | H | H |

| | | | | | | |
|---|---|---|---|---|---|---|
| i-Pr | Me | H | H | —(CH₂)₂— | H | H |

| | | | | | | |
|---|---|---|---|---|---|---|
| i-Pr | Me | H | H | H | H | —(CH₂)₂— |

| | | | | | | |
|---|---|---|---|---|---|---|
| i-Pr | Et | —(CH₂)₂— | H | H | H | H |

| | | | | | | |
|---|---|---|---|---|---|---|
| i-Pr | Et | H | H | —(CH₂)₂— | H | H |

| | | | | | | |
|---|---|---|---|---|---|---|
| i-Pr | Et | H | H | H | H | —(CH₂)₂— |

TABLE L23

| | | | | | | | |
|---|---|---|---|---|---|---|---|
| R¹ | R² | R³ | R⁴ | R⁵ | R⁶ | R⁷ | R⁸ |

| | | | | | | |
|---|---|---|---|---|---|---|
| Pr | H | —(CH₂)₂— | H | H | H | H |

| | | | | | | |
|---|---|---|---|---|---|---|
| Pr | H | H | H | —(CH₂)₂— | H | H |

| | | | | | | |
|---|---|---|---|---|---|---|
| Pr | H | H | H | H | H | —(CH₂)₂— |

| | | | | | | |
|---|---|---|---|---|---|---|
| Pr | Me | —(CH₂)₂— | H | H | H | H |

| | | | | | | |
|---|---|---|---|---|---|---|
| Pr | Me | H | H | —(CH₂)₂— | H | H |

| | | | | | | |
|---|---|---|---|---|---|---|
| Pr | Me | H | H | H | H | —(CH₂)₂— |

| | | | | | | |
|---|---|---|---|---|---|---|
| Pr | Et | —(CH₂)₂— | H | H | H | H |

| | | | | | | |
|---|---|---|---|---|---|---|
| Pr | Et | H | H | —(CH₂)₂— | H | H |

| | | | | | | |
|---|---|---|---|---|---|---|
| Pr | Et | H | H | H | H | —(CH₂)₂— |

| | | | | | | |
|---|---|---|---|---|---|---|
| Pr | i-Pr | —(CH₂)₂— | H | H | H | H |

| | | | | | | |
|---|---|---|---|---|---|---|
| Pr | i-Pr | H | H | —(CH₂)₂— | H | H |

| | | | | | | |
|---|---|---|---|---|---|---|
| Pr | i-Pr | H | H | H | H | —(CH₂)₂— |

| | | | | | | |
|---|---|---|---|---|---|---|
| Pr | Pr | —(CH₂)₂— | H | H | H | H |

| | | | | | | |
|---|---|---|---|---|---|---|
| Pr | Pr | H | H | —(CH₂)₂— | H | H |

| | | | | | | |
|---|---|---|---|---|---|---|
| Pr | Pr | H | H | H | H | —(CH₂)₂— |

| | | | | | | |
|---|---|---|---|---|---|---|
| Bu | H | —(CH₂)₂— | H | H | H | H |

| | | | | | | |
|---|---|---|---|---|---|---|
| Bu | H | H | H | —(CH₂)₂— | H | H |

| | | | | | | |
|---|---|---|---|---|---|---|
| Bu | H | H | H | H | H | —(CH₂)₂— |

| | | | | | | |
|---|---|---|---|---|---|---|
| Bu | Me | —(CH₂)₂— | H | H | H | H |

| | | | | | | |
|---|---|---|---|---|---|---|
| Bu | Me | H | H | —(CH₂)₂— | H | H |

| | | | | | | |
|---|---|---|---|---|---|---|
| Bu | Me | H | H | H | H | —(CH₂)₂— |

| | | | | | | |
|---|---|---|---|---|---|---|
| Bu | Et | —(CH₂)₂— | H | H | H | H |

| | | | | | | |
|---|---|---|---|---|---|---|
| Bu | Et | H | H | —(CH₂)₂— | H | H |

| | | | | | | |
|---|---|---|---|---|---|---|
| Bu | Et | H | H | H | H | —(CH₂)₂— |

| | | | | | | |
|---|---|---|---|---|---|---|
| Bu | i-Pr | —(CH₂)₂— | H | H | H | H |

| | | | | | | |
|---|---|---|---|---|---|---|
| Bu | i-Pr | H | H | —(CH₂)₂— | H | H |

| | | | | | | |
|---|---|---|---|---|---|---|
| Bu | i-Pr | H | H | H | H | —(CH₂)₂— |

| | | | | | | |
|---|---|---|---|---|---|---|
| Bu | Pr | —(CH₂)₂— | H | H | H | H |

| | | | | | | |
|---|---|---|---|---|---|---|
| Bu | Pr | H | H | —(CH₂)₂— | H | H |

| | | | | | | |
|---|---|---|---|---|---|---|
| Bu | Pr | H | H | H | H | —(CH₂)₂— |

| | | | | | | |
|---|---|---|---|---|---|---|
| Bu | Bu | —(CH₂)₂— | H | H | H | H |

| | | | | | | |
|---|---|---|---|---|---|---|
| Bu | Bu | H | H | —(CH₂)₂— | H | H |

| | | | | | | |
|---|---|---|---|---|---|---|
| Bu | Bu | H | H | H | H | —(CH₂)₂— |

TABLE L24

| | | | | | | | |
|---|---|---|---|---|---|---|---|
| R¹ | R² | R³ | R⁴ | R⁵ | R⁶ | R⁷ | R⁸ |

| | | | | | | |
|---|---|---|---|---|---|---|
| t-Bu | H | —(CH₂)₂— | H | H | H | H |

| | | | | | | |
|---|---|---|---|---|---|---|
| t-Bu | H | H | H | —(CH₂)₂— | H | H |

| | | | | | | |
|---|---|---|---|---|---|---|
| t-Bu | H | H | H | H | H | —(CH₂)₂— |

| | | | | | | |
|---|---|---|---|---|---|---|
| t-Bu | Me | —(CH₂)₂— | H | H | H | H |

| | | | | | | |
|---|---|---|---|---|---|---|
| t-Bu | Me | H | H | —(CH₂)₂— | H | H |

| | | | | | | |
|---|---|---|---|---|---|---|
| t-Bu | Me | H | H | H | H | —(CH₂)₂— |

| | | | | | | |
|---|---|---|---|---|---|---|
| t-Bu | Et | —(CH₂)₂— | H | H | H | H |

| | | | | | | |
|---|---|---|---|---|---|---|
| t-Bu | Et | H | H | —(CH₂)₂— | H | H |

| | | | | | | |
|---|---|---|---|---|---|---|
| t-Bu | Et | H | H | H | H | —(CH₂)₂— |

| | | | | | | |
|---|---|---|---|---|---|---|
| t-Bu | Pr | —(CH₂)₂— | H | H | H | H |

| | | | | | | |
|---|---|---|---|---|---|---|
| t-Bu | Pr | H | H | —(CH₂)₂— | H | H |

| | | | | | | |
|---|---|---|---|---|---|---|
| t-Bu | Pr | H | H | H | H | —(CH₂)₂— |

| | | | | | | |
|---|---|---|---|---|---|---|
| t-Bu | Bu | —(CH₂)₂— | H | H | H | H |

| | | | | | | |
|---|---|---|---|---|---|---|
| t-Bu | Bu | H | H | —(CH₂)₂— | H | H |

| | | | | | | |
|---|---|---|---|---|---|---|
| t-Bu | Bu | H | H | H | H | —(CH₂)₂— |

TABLE L25

| | | | | | | | |
|---|---|---|---|---|---|---|---|
| R¹ | R² | R³ | R⁴ | R⁵ | R⁶ | R⁷ | R⁸ |

| | | | | | | |
|---|---|---|---|---|---|---|
| H | H | —(CH₂)₃— | H | H | H | H |

| | | | | | | |
|---|---|---|---|---|---|---|
| H | H | H | H | —(CH₂)₃— | H | H |

| | | | | | | |
|---|---|---|---|---|---|---|
| H | H | H | H | H | H | —(CH₂)₃— |

| | | | | | | |
|---|---|---|---|---|---|---|
| Me | H | —(CH₂)₃— | H | H | H | H |

| | | | | | | |
|---|---|---|---|---|---|---|
| Me | H | H | H | —(CH₂)₃— | H | H |

| | | | | | | |
|---|---|---|---|---|---|---|
| Me | H | H | H | H | H | —(CH₂)₃— |

| | | | | | | |
|---|---|---|---|---|---|---|
| Me | Me | —(CH₂)₃— | H | H | H | H |

| | | | | | | |
|---|---|---|---|---|---|---|
| Me | Me | H | H | —(CH₂)₃— | H | H |

| | | | | | | |
|---|---|---|---|---|---|---|
| Me | Me | H | H | H | H | —(CH₂)₃— |

| | | | | | | |
|---|---|---|---|---|---|---|
| Et | H | —(CH₂)₃— | H | H | H | H |

| | | | | | | |
|---|---|---|---|---|---|---|
| Et | H | H | H | —(CH₂)₃— | H | H |

| | | | | | | |
|---|---|---|---|---|---|---|
| Et | H | H | H | H | H | —(CH₂)₃— |

| | | | | | | |
|---|---|---|---|---|---|---|
| Et | Me | —(CH₂)₃— | H | H | H | H |

| | | | | | | |
|---|---|---|---|---|---|---|
| Et | Me | H | H | —(CH₂)₃— | H | H |

| | | | | | | |
|---|---|---|---|---|---|---|
| Et | Me | H | H | H | H | —(CH₂)₃— |

| | | | | | | |
|---|---|---|---|---|---|---|
| Et | Et | —(CH₂)₃— | H | H | H | H |

| | | | | | | |
|---|---|---|---|---|---|---|
| Et | Et | H | H | —(CH₂)₃— | H | H |

| | | | | | | |
|---|---|---|---|---|---|---|
| Et | Et | H | H | H | H | —(CH₂)₃— |

| | | | | | | |
|---|---|---|---|---|---|---|
| i-Pr | H | —(CH₂)₃— | H | H | H | H |

| | | | | | | |
|---|---|---|---|---|---|---|
| i-Pr | H | H | H | —(CH₂)₃— | H | H |

| | | | | | | |
|---|---|---|---|---|---|---|
| i-Pr | H | H | H | H | H | —(CH₂)₃— |

| | | | | | | |
|---|---|---|---|---|---|---|
| i-Pr | Me | —(CH₂)₃— | H | H | H | H |

| | | | | | | |
|---|---|---|---|---|---|---|
| i-Pr | Me | H | H | —(CH₂)₃— | H | H |

| | | | | | | |
|---|---|---|---|---|---|---|
| i-Pr | Me | H | H | H | H | —(CH₂)₃— |

| | | | | | | |
|---|---|---|---|---|---|---|
| i-Pr | Et | —(CH₂)₃— | H | H | H | H |

| | | | | | | |
|---|---|---|---|---|---|---|
| i-Pr | Et | H | H | —(CH₂)₃— | H | H |

| | | | | | | |
|---|---|---|---|---|---|---|
| i-Pr | Et | H | H | H | H | —(CH₂)₃— |

TABLE L26

| | | | | | | | |
|---|---|---|---|---|---|---|---|
| R¹ | R² | R³ | R⁴ | R⁵ | R⁶ | R⁷ | R⁸ |

| | | | | | | |
|---|---|---|---|---|---|---|
| Pr | H | —(CH₂)₃— | H | H | H | H |

| | | | | | | |
|---|---|---|---|---|---|---|
| Pr | H | H | H | —(CH₂)₃— | H | H |

| | | | | | | |
|---|---|---|---|---|---|---|
| Pr | H | H | H | H | H | —(CH₂)₃— |

| | | | | | | |
|---|---|---|---|---|---|---|
| Pr | Me | —(CH₂)₃— | H | H | H | H |

| | | | | | | |
|---|---|---|---|---|---|---|
| Pr | Me | H | H | —(CH₂)₃— | H | H |

| | | | | | | |
|---|---|---|---|---|---|---|
| Pr | Me | H | H | H | H | —(CH₂)₃— |

| | | | | | | |
|---|---|---|---|---|---|---|
| Pr | Et | —(CH₂)₃— | H | H | H | H |

| | | | | | | |
|---|---|---|---|---|---|---|
| Pr | Et | H | H | —(CH₂)₃— | H | H |

| | | | | | | |
|---|---|---|---|---|---|---|
| Pr | Et | H | H | H | H | —(CH₂)₃— |

| | | | | | | |
|---|---|---|---|---|---|---|
| Pr | i-Pr | —(CH₂)₃— | H | H | H | H |

| | | | | | | |
|---|---|---|---|---|---|---|
| Pr | i-Pr | H | H | —(CH₂)₃— | H | H |

| | | | | | | |
|---|---|---|---|---|---|---|
| Pr | i-Pr | H | H | H | H | —(CH₂)₃— |

| | | | | | | |
|---|---|---|---|---|---|---|
| Pr | Pr | —(CH₂)₃— | H | H | H | H |

| | | | | | | |
|---|---|---|---|---|---|---|
| Pr | Pr | H | H | —(CH₂)₃— | H | H |

| | | | | | | |
|---|---|---|---|---|---|---|
| Pr | Pr | H | H | H | H | —(CH₂)₃— |

| | | | | | | |
|---|---|---|---|---|---|---|
| Bu | H | —(CH₂)₃— | H | H | H | H |

| | | | | | | |
|---|---|---|---|---|---|---|
| Bu | H | H | H | —(CH₂)₃— | H | H |

| | | | | | | |
|---|---|---|---|---|---|---|
| Bu | H | H | H | H | H | —(CH₂)₃— |

| | | | | | | |
|---|---|---|---|---|---|---|
| Bu | Me | —(CH₂)₃— | H | H | H | H |

| | | | | | | |
|---|---|---|---|---|---|---|
| Bu | Me | H | H | —(CH₂)₃— | H | H |

| | | | | | | |
|---|---|---|---|---|---|---|
| Bu | Me | H | H | H | H | —(CH₂)₃— |

| | | | | | | |
|---|---|---|---|---|---|---|
| Bu | Et | —(CH₂)₃— | H | H | H | H |

| | | | | | | |
|---|---|---|---|---|---|---|
| Bu | Et | H | H | —(CH₂)₃— | H | H |

| | | | | | | |
|---|---|---|---|---|---|---|
| Bu | Et | H | H | H | H | —(CH₂)₃— |

| | | | | | | |
|---|---|---|---|---|---|---|
| Bu | i-Pr | —(CH₂)₃— | H | H | H | H |

| | | | | | | |
|---|---|---|---|---|---|---|
| Bu | i-Pr | H | H | —(CH₂)₃— | H | H |

| | | | | | | |
|---|---|---|---|---|---|---|
| Bu | i-Pr | H | H | H | H | —(CH₂)₃— |

| | | | | | | |
|---|---|---|---|---|---|---|
| Bu | Pr | —(CH₂)₃— | H | H | H | H |

| | | | | | | |
|---|---|---|---|---|---|---|
| Bu | Pr | H | H | —(CH₂)₃— | H | H |

| | | | | | | |
|---|---|---|---|---|---|---|
| Bu | Pr | H | H | H | H | —(CH₂)₃— |

| | | | | | | |
|---|---|---|---|---|---|---|
| Bu | Bu | —(CH₂)₃— | H | H | H | H |

| | | | | | | |
|---|---|---|---|---|---|---|
| Bu | Bu | H | H | —(CH₂)₃— | H | H |

| | | | | | | |
|---|---|---|---|---|---|---|
| Bu | Bu | H | H | H | H | —(CH₂)₃— |

TABLE L27

| | | | | | | | |
|---|---|---|---|---|---|---|---|
| R¹ | R² | R³ | R⁴ | R⁵ | R⁶ | R⁷ | R⁸ |

| | | | | | | |
|---|---|---|---|---|---|---|
| t-Bu | H | —(CH₂)₃— | H | H | H | H |

| | | | | | | |
|---|---|---|---|---|---|---|
| t-Bu | H | H | H | —(CH₂)₃— | H | H |

| | | | | | | |
|---|---|---|---|---|---|---|
| t-Bu | H | H | H | H | H | —(CH₂)₃— |

| | | | | | | |
|---|---|---|---|---|---|---|
| t-Bu | Me | —(CH₂)₃— | H | H | H | H |

| | | | | | | |
|---|---|---|---|---|---|---|
| t-Bu | Me | H | H | —(CH₂)₃— | H | H |

| | | | | | | |
|---|---|---|---|---|---|---|
| t-Bu | Me | H | H | H | H | —(CH₂)₃— |

| | | | | | | |
|---|---|---|---|---|---|---|
| t-Bu | Et | —(CH₂)₃— | H | H | H | H |

| | | | | | | |
|---|---|---|---|---|---|---|
| t-Bu | Et | H | H | —(CH₂)₃— | H | H |

| | | | | | | |
|---|---|---|---|---|---|---|
| t-Bu | Et | H | H | H | H | —(CH₂)₃— |

| | | | | | | |
|---|---|---|---|---|---|---|
| t-Bu | Pr | —(CH₂)₃— | H | H | H | H |

| | | | | | |
|---|---|---|---|---|---|
| t-Bu | Pr | H | H | —(CH₂)₃— | H |

| | | | | | | |
|---|---|---|---|---|---|---|
| t-Bu | Pr | H | H | H | H | —(CH₂)₃— |

| | | | | | | |
|---|---|---|---|---|---|---|
| t-Bu | Bu | —(CH₂)₃— | H | H | H | H |

| | | | | | | |
|---|---|---|---|---|---|---|
| t-Bu | Bu | H | H | —(CH₂)₃— | H | H |

| | | | | | | |
|---|---|---|---|---|---|---|
| t-Bu | Bu | H | H | H | H | —(CH₂)₃— |

TABLE L28

| | | | | | | | |
|---|---|---|---|---|---|---|---|
| R¹ | R² | R³ | R⁴ | R⁵ | R⁶ | R⁷ | R⁸ |

| | | | | | | |
|---|---|---|---|---|---|---|
| H | H | —(CH₂)₄— | H | H | H | H |

| | | | | | | |
|---|---|---|---|---|---|---|
| H | H | H | H | —(CH₂)₄— | H | H |

| | | | | | | |
|---|---|---|---|---|---|---|
| H | H | H | H | H | H | —(CH₂)₄— |

| | | | | | | |
|---|---|---|---|---|---|---|
| Me | H | —(CH₂)₄— | H | H | H | H |

| | | | | | | |
|---|---|---|---|---|---|---|
| Me | H | H | H | —(CH₂)₄— | H | H |

| | | | | | | |
|---|---|---|---|---|---|---|
| Me | H | H | H | H | H | —(CH₂)₄— |

| | | | | | | |
|---|---|---|---|---|---|---|
| Me | Me | —(CH₂)₄— | H | H | H | H |

| | | | | | | |
|---|---|---|---|---|---|---|
| Me | Me | H | H | —(CH₂)₄— | H | H |

| | | | | | | |
|---|---|---|---|---|---|---|
| Me | Me | H | H | H | H | —(CH₂)₄— |

| | | | | | | |
|---|---|---|---|---|---|---|
| Et | H | —(CH₂)₄— | H | H | H | H |

| | | | | | | |
|---|---|---|---|---|---|---|
| Et | H | H | H | —(CH₂)₄— | H | H |

| | | | | | | |
|---|---|---|---|---|---|---|
| Et | H | H | H | H | H | —(CH₂)₄— |

| | | | | | | |
|---|---|---|---|---|---|---|
| Et | Me | —(CH₂)₄— | H | H | H | H |

| | | | | | | |
|---|---|---|---|---|---|---|
| Et | Me | H | H | —(CH₂)₄— | H | H |

| | | | | | | |
|---|---|---|---|---|---|---|
| Et | Me | H | H | H | H | —(CH₂)₄— |

| | | | | | | |
|---|---|---|---|---|---|---|
| Et | Et | —(CH₂)₄— | H | H | H | H |

| | | | | | | |
|---|---|---|---|---|---|---|
| Et | Et | H | H | —(CH₂)₄— | H | H |

| | | | | | | |
|---|---|---|---|---|---|---|
| Et | Et | H | H | H | H | —(CH₂)₄— |

| | | | | | | |
|---|---|---|---|---|---|---|
| i-Pr | H | —(CH₂)₄— | H | H | H | H |

| | | | | | | |
|---|---|---|---|---|---|---|
| i-Pr | H | H | H | —(CH₂)₄— | H | H |

| | | | | | | |
|---|---|---|---|---|---|---|
| i-Pr | H | H | H | H | H | —(CH₂)₄— |

| | | | | | | |
|---|---|---|---|---|---|---|
| i-Pr | Me | —(CH₂)₄— | H | H | H | H |

| | | | | | | |
|---|---|---|---|---|---|---|
| i-Pr | Me | H | H | —(CH₂)₄— | H | H |

| | | | | | | |
|---|---|---|---|---|---|---|
| i-Pr | Me | H | H | H | H | —(CH₂)₄— |

| | | | | | | |
|---|---|---|---|---|---|---|
| i-Pr | Et | —(CH₂)₄— | H | H | H | H |

| | | | | | | |
|---|---|---|---|---|---|---|
| i-Pr | Et | H | H | —(CH₂)₄— | H | H |

| | | | | | | |
|---|---|---|---|---|---|---|
| i-Pr | Et | H | H | H | H | —(CH₂)₄— |

TABLE L29

| | | | | | | | |
|---|---|---|---|---|---|---|---|
| R¹ | R² | R³ | R⁴ | R⁵ | R⁶ | R⁷ | R⁸ |

| | | | | | | |
|---|---|---|---|---|---|---|
| Pr | H | —(CH₂)₄— | H | H | H | H |

| | | | | | | |
|---|---|---|---|---|---|---|
| Pr | H | H | H | —(CH₂)₄— | H | H |

| | | | | | | |
|---|---|---|---|---|---|---|
| Pr | H | H | H | H | H | —(CH₂)₄— |

| | | | | | | |
|---|---|---|---|---|---|---|
| Pr | Me | —(CH₂)₄— | H | H | H | H |

| | | | | | | |
|---|---|---|---|---|---|---|
| Pr | Me | H | H | —(CH₂)₄— | H | H |

| | | | | | | |
|---|---|---|---|---|---|---|
| Pr | Me | H | H | H | H | —(CH₂)₄— |

| | | | | | | |
|---|---|---|---|---|---|---|
| Pr | Et | —(CH₂)₄— | H | H | H | H |

| | | | | | | |
|---|---|---|---|---|---|---|
| Pr | Et | H | H | —(CH₂)₄— | H | H |

| | | | | | | |
|---|---|---|---|---|---|---|
| Pr | Et | H | H | H | H | —(CH₂)₄— |

| | | | | | | |
|---|---|---|---|---|---|---|
| Pr | i-Pr | —(CH₂)₄— | H | H | H | H |

| | | | | | | |
|---|---|---|---|---|---|---|
| Pr | i-Pr | H | H | —(CH₂)₄— | H | H |

| | | | | | | |
|---|---|---|---|---|---|---|
| Pr | i-Pr | H | H | H | H | —(CH₂)₄— |

| | | | | | | |
|---|---|---|---|---|---|---|
| Pr | Pr | —(CH₂)₄— | H | H | H | H |

| | | | | | | |
|---|---|---|---|---|---|---|
| Pr | Pr | H | H | —(CH₂)₄— | H | H |

| | | | | | | |
|---|---|---|---|---|---|---|
| Pr | Pr | H | H | H | H | —(CH₂)₄— |

| | | | | | | |
|---|---|---|---|---|---|---|
| Bu | H | —(CH₂)₄— | H | H | H | H |

| | | | | | | |
|---|---|---|---|---|---|---|
| Bu | H | H | H | —(CH₂)₄— | H | H |

| | | | | | | |
|---|---|---|---|---|---|---|
| Bu | H | H | H | H | H | —(CH₂)₄— |

| | | | | | | |
|---|---|---|---|---|---|---|
| Bu | Me | —(CH₂)₄— | H | H | H | H |

| | | | | | | |
|---|---|---|---|---|---|---|
| Bu | Me | H | H | —(CH₂)₄— | H | H |

| | | | | | | |
|---|---|---|---|---|---|---|
| Bu | Me | H | H | H | H | —(CH₂)₄— |

| | | | | | | |
|---|---|---|---|---|---|---|
| Bu | Et | —(CH₂)₄— | H | H | H | H |

| | | | | | | |
|---|---|---|---|---|---|---|
| Bu | Et | H | H | —(CH₂)₄— | H | H |

| | | | | | | |
|---|---|---|---|---|---|---|
| Bu | Et | H | H | H | H | —(CH₂)₄— |

| | | | | | | |
|---|---|---|---|---|---|---|
| Bu | i-Pr | —(CH₂)₄— | H | H | H | H |

| | | | | | | |
|---|---|---|---|---|---|---|
| Bu | i-Pr | H | H | —(CH₂)₄— | H | H |

| | | | | | | |
|---|---|---|---|---|---|---|
| Bu | i-Pr | H | H | H | H | —(CH₂)₄— |

| | | | | | | |
|---|---|---|---|---|---|---|
| Bu | Pr | —(CH₂)₄— | H | H | H | H |

| | | | | | | |
|---|---|---|---|---|---|---|
| Bu | Pr | H | H | —(CH₂)₄— | H | H |

| | | | | | | |
|---|---|---|---|---|---|---|
| Bu | Pr | H | H | H | H | —(CH₂)₄— |

| | | | | | | |
|---|---|---|---|---|---|---|
| Bu | Bu | —(CH₂)₄— | H | H | H | H |

| | | | | | | |
|---|---|---|---|---|---|---|
| Bu | Bu | H | H | —(CH₂)₄— | H | H |

| | | | | | | |
|---|---|---|---|---|---|---|
| Bu | Bu | H | H | H | H | —(CH₂)₄— |

TABLE L30

| | | | | | | | |
|---|---|---|---|---|---|---|---|
| R¹ | R² | R³ | R⁴ | R⁵ | R⁶ | R⁷ | R⁸ |

| | | | | | | |
|---|---|---|---|---|---|---|
| t-Bu | H | —(CH₂)₄— | H | H | H | H |

| | | | | | | |
|---|---|---|---|---|---|---|
| t-Bu | H | H | H | —(CH₂)₄— | H | H |

| | | | | | | |
|---|---|---|---|---|---|---|
| t-Bu | H | H | H | H | H | —(CH₂)₄— |

| | | | | | | |
|---|---|---|---|---|---|---|
| t-Bu | Me | —(CH₂)₄— | H | H | H | H |

| | | | | | | |
|---|---|---|---|---|---|---|
| t-Bu | Me | H | H | —(CH₂)₄— | H | H |

| | | | | | | |
|---|---|---|---|---|---|---|
| t-Bu | Me | H | H | H | H | —(CH₂)₄— |

| | | | | | | |
|---|---|---|---|---|---|---|
| t-Bu | Et | —(CH₂)₄— | H | H | H | H |

| | | | | | | |
|---|---|---|---|---|---|---|
| t-Bu | Et | H | H | —(CH₂)₄— | H | H |

| | | | | | | |
|---|---|---|---|---|---|---|
| t-Bu | Et | H | H | H | H | —(CH₂)₄— |

| | | | | | | |
|---|---|---|---|---|---|---|
| t-Bu | Pr | —(CH₂)₄— | H | H | H | H |

| | | | | | | |
|---|---|---|---|---|---|---|
| t-Bu | Pr | H | H | —(CH₂)₄— | H | H |

| | | | | | | |
|---|---|---|---|---|---|---|
| t-Bu | Pr | H | H | H | H | —(CH₂)₄— |

| | | | | | | |
|---|---|---|---|---|---|---|
| t-Bu | Bu | —(CH₂)₄— | H | H | H | H |

| | | | | | | |
|---|---|---|---|---|---|---|
| t-Bu | Bu | H | H | —(CH₂)₄— | H | H |

| | | | | | | |
|---|---|---|---|---|---|---|
| t-Bu | Bu | H | H | H | H | —(CH₂)₄— |

TABLE L31

| | | | | | | | |
|---|---|---|---|---|---|---|---|
| R¹ | R² | R³ | R⁴ | R⁵ | R⁶ | R⁷ | R⁸ |

| | | | | | | |
|---|---|---|---|---|---|---|
| H | H | —(CH₂)₅— | H | H | H | H |

| | | | | | | |
|---|---|---|---|---|---|---|
| H | H | H | H | —(CH₂)₅— | H | H |

| | | | | | | |
|---|---|---|---|---|---|---|
| H | H | H | H | H | H | —(CH₂)₅— |

| | | | | | | |
|---|---|---|---|---|---|---|
| Me | H | —(CH₂)₅— | H | H | H | H |

| | | | | | | |
|---|---|---|---|---|---|---|
| Me | H | H | H | —(CH₂)₅— | H | H |

| | | | | | | |
|---|---|---|---|---|---|---|
| Me | H | H | H | H | H | —(CH₂)₅— |

| | | | | | | |
|---|---|---|---|---|---|---|
| Me | Me | —(CH₂)₅— | H | H | H | H |

| | | | | | | |
|---|---|---|---|---|---|---|
| Me | Me | H | H | —(CH₂)₅— | H | H |

| | | | | | | |
|---|---|---|---|---|---|---|
| Me | Me | H | H | H | H | —(CH₂)₅— |

| | | | | | | |
|---|---|---|---|---|---|---|
| Et | H | —(CH₂)₅— | H | H | H | H |

| | | | | | | |
|---|---|---|---|---|---|---|
| Et | H | H | H | —(CH₂)₅— | H | H |

| | | | | | | |
|---|---|---|---|---|---|---|
| Et | H | H | H | H | H | —(CH₂)₅— |

| | | | | | | |
|---|---|---|---|---|---|---|
| Et | Me | —(CH₂)₅— | H | H | H | H |

| | | | | | | |
|---|---|---|---|---|---|---|
| Et | Me | H | H | —(CH₂)₅— | H | H |

| | | | | | | |
|---|---|---|---|---|---|---|
| Et | Me | H | H | H | H | —(CH₂)₅— |

| | | | | | | |
|---|---|---|---|---|---|---|
| Et | Et | —(CH₂)₅— | H | H | H | H |

| | | | | | | |
|---|---|---|---|---|---|---|
| Et | Et | H | H | —(CH₂)₅— | H | H |

| | | | | | | |
|---|---|---|---|---|---|---|
| Et | Et | H | H | H | H | —(CH₂)₅— |

| | | | | | | |
|---|---|---|---|---|---|---|
| i-Pr | H | —(CH₂)₅— | H | H | H | H |

| | | | | | | |
|---|---|---|---|---|---|---|
| i-Pr | H | H | H | —(CH₂)₅— | H | H |

| | | | | | | |
|---|---|---|---|---|---|---|
| i-Pr | H | H | H | H | H | —(CH₂)₅— |

| | | | | | | |
|---|---|---|---|---|---|---|
| i-Pr | Me | —(CH₂)₅— | H | H | H | H |

| | | | | | | |
|---|---|---|---|---|---|---|
| i-Pr | Me | H | H | —(CH₂)₅— | H | H |

| | | | | | | |
|---|---|---|---|---|---|---|
| i-Pr | Me | H | H | H | H | —(CH₂)₅— |

| | | | | | | |
|---|---|---|---|---|---|---|
| i-Pr | Et | —(CH₂)₅— | H | H | H | H |

| | | | | | | |
|---|---|---|---|---|---|---|
| i-Pr | Et | H | H | —(CH₂)₅— | H | H |

| | | | | | | |
|---|---|---|---|---|---|---|
| i-Pr | Et | H | H | H | H | —(CH₂)₅— |

TABLE L32

| | | | | | | | |
|---|---|---|---|---|---|---|---|
| R¹ | R² | R³ | R⁴ | R⁵ | R⁶ | R⁷ | R⁸ |

| | | | | | | |
|---|---|---|---|---|---|---|
| Pr | H | —(CH₂)₅— | H | H | H | H |

| | | | | | | |
|---|---|---|---|---|---|---|
| Pr | H | H | H | —(CH₂)₅— | H | H |

| | | | | | | |
|---|---|---|---|---|---|---|
| Pr | H | H | H | H | H | —(CH₂)₅— |

| | | | | | | |
|---|---|---|---|---|---|---|
| Pr | Me | —(CH₂)₅— | H | H | H | H |

| | | | | | | |
|---|---|---|---|---|---|---|
| Pr | Me | H | H | —(CH₂)₅— | H | H |

| | | | | | | |
|---|---|---|---|---|---|---|
| Pr | Me | H | H | H | H | —(CH₂)₅— |

| | | | | | | |
|---|---|---|---|---|---|---|
| Pr | Et | —(CH₂)₅— | H | H | H | H |

| | | | | | | |
|---|---|---|---|---|---|---|
| Pr | Et | H | H | —(CH₂)₅— | H | H |

| | | | | | | |
|---|---|---|---|---|---|---|
| Pr | Et | H | H | H | H | —(CH₂)₅— |

| | | | | | | |
|---|---|---|---|---|---|---|
| Pr | i-Pr | —(CH₂)₅— | H | H | H | H |

| | | | | | | |
|---|---|---|---|---|---|---|
| Pr | i-Pr | H | H | —(CH₂)₅— | H | H |

| | | | | | | |
|---|---|---|---|---|---|---|
| Pr | i-Pr | H | H | H | H | —(CH₂)₅— |

| | | | | | | |
|---|---|---|---|---|---|---|
| Pr | Pr | —(CH₂)₅— | H | H | H | H |

| | | | | | | |
|---|---|---|---|---|---|---|
| Pr | Pr | H | H | —(CH₂)₅— | H | H |

| | | | | | | |
|---|---|---|---|---|---|---|
| Pr | Pr | H | H | H | H | —(CH₂)₅— |

| | | | | | | |
|---|---|---|---|---|---|---|
| Bu | H | —(CH₂)₅— | H | H | H | H |

| | | | | | | |
|---|---|---|---|---|---|---|
| Bu | H | H | H | —(CH₂)₅— | H | H |

| | | | | | | |
|---|---|---|---|---|---|---|
| Bu | H | H | H | H | H | —(CH₂)₅— |

| | | | | | | |
|---|---|---|---|---|---|---|
| Bu | Me | —(CH₂)₅— | H | H | H | H |

| | | | | | | |
|---|---|---|---|---|---|---|
| Bu | Me | H | H | —(CH₂)₅— | H | H |

| | | | | | | |
|---|---|---|---|---|---|---|
| Bu | Me | H | H | H | H | —(CH₂)₅— |

| | | | | | | |
|---|---|---|---|---|---|---|
| Bu | Et | —(CH₂)₅— | H | H | H | H |

| | | | | | | |
|---|---|---|---|---|---|---|
| Bu | Et | H | H | —(CH₂)₅— | H | H |

| | | | | | | |
|---|---|---|---|---|---|---|
| Bu | Et | H | H | H | H | —(CH₂)₅— |

| | | | | | | |
|---|---|---|---|---|---|---|
| Bu | i-Pr | —(CH₂)₅— | H | H | H | H |

| | | | | | | |
|---|---|---|---|---|---|---|
| Bu | i-Pr | H | H | —(CH₂)₅— | H | H |

| | | | | | | |
|---|---|---|---|---|---|---|
| Bu | i-Pr | H | H | H | H | —(CH₂)₅— |

| | | | | | | |
|---|---|---|---|---|---|---|
| Bu | Pr | —(CH₂)₅— | H | H | H | H |

| | | | | | | |
|---|---|---|---|---|---|---|
| Bu | Pr | H | H | —(CH₂)₅— | H | H |

| | | | | | | |
|---|---|---|---|---|---|---|
| Bu | Pr | H | H | H | H | —(CH₂)₅— |

| | | | | | | |
|---|---|---|---|---|---|---|
| Bu | Bu | —(CH₂)₅— | H | H | H | H |

| | | | | | | |
|---|---|---|---|---|---|---|
| Bu | Bu | H | H | —(CH₂)₅— | H | H |

| | | | | | | |
|---|---|---|---|---|---|---|
| Bu | Bu | H | H | H | H | —(CH₂)₅— |

TABLE L33

| | | | | | | | |
|---|---|---|---|---|---|---|---|
| R¹ | R² | R³ | R⁴ | R⁵ | R⁶ | R⁷ | R⁸ |

| | | | | | | |
|---|---|---|---|---|---|---|
| t-Bu | H | —(CH₂)₅— | H | H | H | H |

| | | | | | | |
|---|---|---|---|---|---|---|
| t-Bu | H | H | H | —(CH₂)₅— | H | H |

| | | | | | | |
|---|---|---|---|---|---|---|
| t-Bu | H | H | H | H | H | —(CH₂)₅— |

| | | | | | | |
|---|---|---|---|---|---|---|
| t-Bu | Me | —(CH₂)₅— | H | H | H | H |

| | | | | | | |
|---|---|---|---|---|---|---|
| t-Bu | Me | H | H | —(CH₂)₅— | H | H |

| | | | | | | |
|---|---|---|---|---|---|---|
| t-Bu | Me | H | H | H | H | —(CH₂)₅— |

| | | | | | | |
|---|---|---|---|---|---|---|
| t-Bu | Et | —(CH₂)₅— | H | H | H | H |

| | | | | | | |
|---|---|---|---|---|---|---|
| t-Bu | Et | H | H | —(CH₂)₅— | H | H |

| | | | | | | |
|---|---|---|---|---|---|---|
| t-Bu | Et | H | H | H | H | —(CH₂)₅— |

| | | | | | | |
|---|---|---|---|---|---|---|
| t-Bu | Pr | —(CH₂)₅— | H | H | H | H |

| | | | | | | |
|---|---|---|---|---|---|---|
| t-Bu | Pr | H | H | —(CH₂)₅— | H | H |

| | | | | | | |
|---|---|---|---|---|---|---|
| t-Bu | Pr | H | H | H | H | —(CH₂)₅— |

| | | | | | | |
|---|---|---|---|---|---|---|
| t-Bu | Bu | —(CH₂)₅— | H | H | H | H |

| | | | | | | |
|---|---|---|---|---|---|---|
| t-Bu | Bu | H | H | —(CH₂)₅— | H | H |

| | | | | | | |
|---|---|---|---|---|---|---|
| t-Bu | Bu | H | H | H | H | —(CH₂)₅— |

TABLE L34

| R¹ | R² | R³ | R⁴ | R⁵ | R⁶ | R⁷ | R⁸ |
|---|---|---|---|---|---|---|---|
| CH₂CH═CH₂ | H | Me | Me | H | H | H | H |
| CH₂CH═CH₂ | H | H | H | Me | Me | H | H |
| CH₂CH═CH₂ | H | H | H | H | H | Me | Me |
| CH₂CH═CH₂ | Me | Me | Me | H | H | H | H |
| CH₂CH═CH₂ | Me | H | H | Me | Me | H | H |
| CH₂CH═CH₂ | Me | H | H | H | H | Me | Me |
| CH₂CH═CH₂ | Et | Me | Me | H | H | H | H |
| CH₂CH═CH₂ | Et | H | H | Me | Me | H | H |
| CH₂CH═CH₂ | Et | H | H | H | H | Me | Me |
| CH₂CH═CH₂ | i-Pr | Me | Me | H | H | H | H |
| CH₂CH═CH₂ | i-Pr | H | H | Me | Me | H | H |
| CH₂CH═CH₂ | i-Pr | H | H | H | H | Me | Me |
| CH₂CH═CH₂ | Pr | Me | Me | H | H | H | H |
| CH₂CH═CH₂ | Pr | H | H | Me | Me | H | H |
| CH₂CH═CH₂ | Pr | H | H | H | H | Me | Me |
| CH₂CH═CH₂ | Bu | Me | Me | H | H | H | H |
| CH₂CH═CH₂ | Bu | H | H | Me | Me | H | H |
| CH₂CH═CH₂ | Bu | H | H | H | H | Me | Me |
| CH₂CH═CHCH₃ | H | Me | Me | H | H | H | H |
| CH₂CH═CHCH₃ | H | H | H | Me | Me | H | H |
| CH₂CH═CHCH₃ | H | H | H | H | H | Me | Me |
| CH₂CH═CHCH₃ | Me | Me | Me | H | H | H | H |
| CH₂CH═CHCH₃ | Me | H | H | Me | Me | H | H |
| CH₂CH═CHCH₃ | Me | H | H | H | H | Me | Me |
| CH₂CH═CHCH₃ | Et | Me | Me | H | H | H | H |
| CH₂CH═CHCH₃ | Et | H | H | Me | Me | H | H |
| CH₂CH═CHCH₃ | Et | H | H | H | H | Me | Me |
| CH₂CH═CHCH₃ | i-Pr | Me | Me | H | H | H | H |
| CH₂CH═CHCH₃ | i-Pr | H | H | Me | Me | H | H |
| CH₂CH═CHCH₃ | i-Pr | H | H | H | H | Me | Me |
| CH₂CH═CHCH₃ | Pr | Me | Me | H | H | H | H |
| CH₂CH═CHCH₃ | Pr | H | H | Me | Me | H | H |
| CH₂CH═CHCH₃ | Pr | H | H | H | H | Me | Me |
| CH₂CH═CHCH₃ | Bu | Me | Me | H | H | H | H |
| CH₂CH═CHCH₃ | Bu | H | H | Me | Me | H | H |
| CH₂CH═CHCH₃ | Bu | H | H | H | H | Me | Me |

TABLE L35

| R¹ | R² | R³ | R⁴ | R⁵ | R⁶ | R⁷ | R⁸ |
|---|---|---|---|---|---|---|---|
| CH₂CH═C(CH₃)₂ | H | Me | Me | H | H | H | H |
| CH₂CH═C(CH₃)₂ | H | H | H | Me | Me | H | H |
| CH₂CH═C(CH₃)₂ | H | H | H | H | H | Me | Me |
| CH₂CH═C(CH₃)₂ | Me | Me | Me | H | H | H | H |
| CH₂CH═C(CH₃)₂ | Me | H | H | Me | Me | H | H |
| CH₂CH═C(CH₃)₂ | Me | H | H | H | H | Me | Me |
| CH₂CH═C(CH₃)₂ | Et | Me | Me | H | H | H | H |
| CH₂CH═C(CH₃)₂ | Et | H | H | Me | Me | H | H |
| CH₂CH═C(CH₃)₂ | Et | H | H | H | H | Me | Me |
| CH₂CH═C(CH₃)₂ | i-Pr | Me | Me | H | H | H | H |
| CH₂CH═C(CH₃)₂ | i-Pr | H | H | Me | Me | H | H |
| CH₂CH═C(CH₃)₂ | i-Pr | H | H | H | H | Me | Me |
| CH₂CH═C(CH₃)₂ | Pr | Me | Me | H | H | H | H |
| CH₂CH═C(CH₃)₂ | Pr | H | H | Me | Me | H | H |
| CH₂CH═C(CH₃)₂ | Pr | H | H | H | H | Me | Me |
| CH₂CH═C(CH₃)₂ | Bu | Me | Me | H | H | H | H |
| CH₂CH═C(CH₃)₂ | Bu | H | H | Me | Me | H | H |
| CH₂CH═C(CH₃)₂ | Bu | H | H | H | H | Me | Me |
| CH₂C(CH₃)═CH₂ | H | Me | Me | H | H | H | H |
| CH₂C(CH₃)═CH₂ | H | H | H | Me | Me | H | H |
| CH₂C(CH₃)═CH₂ | H | H | H | H | H | Me | Me |
| CH₂C(CH₃)═CH₂ | Me | Me | Me | H | H | H | H |
| CH₂C(CH₃)═CH₂ | Me | H | H | Me | Me | H | H |
| CH₂C(CH₃)═CH₂ | Me | H | H | H | H | Me | Me |
| CH₂C(CH₃)═CH₂ | Et | Me | Me | H | H | H | H |
| CH₂C(CH₃)═CH₂ | Et | H | H | Me | Me | H | H |
| CH₂C(CH₃)═CH₂ | Et | H | H | H | H | Me | Me |
| CH₂C(CH₃)═CH₂ | i-Pr | Me | Me | H | H | H | H |
| CH₂C(CH₃)═CH₂ | i-Pr | H | H | Me | Me | H | H |
| CH₂C(CH₃)═CH₂ | i-Pr | H | H | H | H | Me | Me |
| CH₂C(CH₃)═CH₂ | Pr | Me | Me | H | H | H | H |
| CH₂C(CH₃)═CH₂ | Pr | H | H | Me | Me | H | H |
| CH₂C(CH₃)═CH₂ | Pr | H | H | H | H | Me | Me |
| CH₂C(CH₃)═CH₂ | Bu | Me | Me | H | H | H | H |
| CH₂C(CH₃)═CH₂ | Bu | H | H | Me | Me | H | H |
| CH₂C(CH₃)═CH₂ | Bu | H | H | H | H | Me | Me |

TABLE L36

| R¹ | R² | R³ | R⁴ | R⁵ | R⁶ | R⁷ | R⁸ |
|---|---|---|---|---|---|---|---|
| CH₂C(CH₃)═CHCH₃ | H | Me | Me | H | H | H | H |
| CH₂C(CH₃)═CHCH₃ | H | H | H | Me | Me | H | H |
| CH₂C(CH₃)═CHCH₃ | H | H | H | H | H | Me | Me |
| CH₂C(CH₃)═CHCH₃ | Me | Me | Me | H | H | H | H |
| CH₂C(CH₃)═CHCH₃ | Me | H | H | Me | Me | H | H |
| CH₂C(CH₃)═CHCH₃ | Me | H | H | H | H | Me | Me |
| CH₂C(CH₃)═CHCH₃ | Et | Me | Me | H | H | H | H |
| CH₂C(CH₃)═CHCH₃ | Et | H | H | Me | Me | H | H |
| CH₂C(CH₃)═CHCH₃ | Et | H | H | H | H | Me | Me |
| CH₂C(CH₃)═CHCH₃ | i-Pr | Me | Me | H | H | H | H |
| CH₂C(CH₃)═CHCH₃ | i-Pr | H | H | Me | Me | H | H |
| CH₂C(CH₃)═CHCH₃ | i-Pr | H | H | H | H | Me | Me |
| CH₂C(CH₃)═CHCH₃ | Pr | Me | Me | H | H | H | H |
| CH₂C(CH₃)═CHCH₃ | Pr | H | H | Me | Me | H | H |
| CH₂C(CH₃)═CHCH₃ | Pr | H | H | H | H | Me | Me |
| CH₂C(CH₃)═CHCH₃ | Bu | Me | Me | H | H | H | H |
| CH₂C(CH₃)═CHCH₃ | Bu | H | H | Me | Me | H | H |
| CH₂C(CH₃)═CHCH₃ | Bu | H | H | H | H | Me | Me |
| CH₂C(CH₃)═C(CH₃)₂ | H | Me | Me | H | H | H | H |
| CH₂C(CH₃)═C(CH₃)₂ | H | H | H | Me | Me | H | H |
| CH₂C(CH₃)═C(CH₃)₂ | H | H | H | H | H | Me | Me |
| CH₂C(CH₃)═C(CH₃)₂ | Me | Me | Me | H | H | H | H |
| CH₂C(CH₃)═C(CH₃)₂ | Me | H | H | Me | Me | H | H |
| CH₂C(CH₃)═C(CH₃)₂ | Me | H | H | H | H | Me | Me |
| CH₂C(CH₃)═C(CH₃)₂ | Et | Me | Me | H | H | H | H |
| CH₂C(CH₃)═C(CH₃)₂ | Et | H | H | Me | Me | H | H |
| CH₂C(CH₃)═C(CH₃)₂ | Et | H | H | H | H | Me | Me |
| CH₂C(CH₃)═C(CH₃)₂ | i-Pr | Me | Me | H | H | H | H |
| CH₂C(CH₃)═C(CH₃)₂ | i-Pr | H | H | Me | Me | H | H |
| CH₂C(CH₃)═C(CH₃)₂ | i-Pr | H | H | H | H | Me | Me |
| CH₂C(CH₃)═C(CH₃)₂ | Pr | Me | Me | H | H | H | H |
| CH₂C(CH₃)═C(CH₃)₂ | Pr | H | H | Me | Me | H | H |
| CH₂C(CH₃)═C(CH₃)₂ | Pr | H | H | H | H | Me | Me |
| CH₂C(CH₃)═C(CH₃)₂ | Bu | Me | Me | H | H | H | H |
| CH₂C(CH₃)═C(CH₃)₂ | Bu | H | H | Me | Me | H | H |
| CH₂C(CH₃)═C(CH₃)₂ | Bu | H | H | H | H | Me | Me |

TABLE L37

| R¹ | R² | R³ | R⁴ | R⁵ | R⁶ | R⁷ | R⁸ |
|---|---|---|---|---|---|---|---|
| CH₂C═CH | H | Me | Me | H | H | H | H |
| CH₂C═CH | H | H | H | Me | Me | H | H |
| CH₂C═CH | H | H | H | H | H | Me | Me |
| CH₂C═CH | Me | Me | Me | H | H | H | H |
| CH₂C═CH | Me | H | H | Me | Me | H | H |
| CH₂C═CH | Me | H | H | H | H | Me | Me |
| CH₂C═CH | Et | Me | Me | H | H | H | H |
| CH₂C═CH | Et | H | H | Me | Me | H | H |
| CH₂C═CH | Et | H | H | H | H | Me | Me |
| CH₂C═CH | i-Pr | Me | Me | H | H | H | H |
| CH₂C═CH | i-Pr | H | H | Me | Me | H | H |
| CH₂C═CH | i-Pr | H | H | H | H | Me | Me |
| CH₂C═CH | Pr | Me | Me | H | H | H | H |
| CH₂C═CH | Pr | H | H | Me | Me | H | H |
| CH₂C═CH | Pr | H | H | H | H | Me | Me |
| CH₂C═CH | Bu | Me | Me | H | H | H | H |
| CH₂C═CH | Bu | H | H | Me | M? | H | H |
| CH₂C═CH | Bu | H | H | H | H | Me | Me |
| CH₂C═CCH₃ | H | Me | Me | H | H | H | H |
| CH₂C═CCH₃ | H | H | H | Me | Me | H | H |
| CH₂C═CCH₃ | H | H | H | H | H | Me | Me |
| CH₂C═CCH₃ | Me | Me | Me | H | H | H | H |
| CH₂C═CCH₃ | Me | H | H | Me | Me | H | H |
| CH₂C═CCH₃ | Me | H | H | H | H | Me | Me |
| CH₂C═CCH₃ | Et | Me | Me | H | H | H | H |
| CH₂C═CCH₃ | Et | H | H | Me | Me | H | H |
| CH₂C═CCH₃ | Et | H | H | H | H | Me | Me |
| CH₂C═CCH₃ | i-Pr | Me | Me | H | H | H | H |
| CH₂C═CCH₃ | i-Pr | H | H | Me | Me | H | H |
| CH₂C═CCH₃ | i-Pr | H | H | H | H | Me | Me |
| CH₂C═CCH₃ | Pr | Me | Me | H | H | H | H |
| CH₂C═CCH₃ | Pr | H | H | Me | Me | H | H |
| CH₂C═CCH₃ | Pr | H | H | H | H | Me | Me |
| CH₂C═CCH₃ | Bu | Me | Me | H | H | H | H |
| CH₂C═CCH₃ | Bu | H | H | Me | Me | H | H |
| CH₂C═CCH₃ | Bu | H | H | H | H | Me | Me |

TABLE L38

| R¹ | R² | R³ | R⁴ | R⁵ | R⁶ | R⁷ | R⁸ |
|---|---|---|---|---|---|---|---|
| CH₂C═CH₂CH₃ | H | Me | Me | H | H | H | H |
| CH₂C═CH₂CH₃ | H | H | H | Me | Me | H | H |
| CH₂C═CH₂CH₃ | H | H | H | H | H | Me | Me |
| CH₂C═CH₂CH₃ | Me | Me | Me | H | H | H | H |
| CH₂C═CH₂CH₃ | Me | H | H | Me | Me | H | H |
| CH₂C═CH₂CH₃ | Me | H | H | H | H | Me | Me |
| CH₂C═CH₂CH₃ | Et | Me | Me | H | H | H | H |
| CH₂C═CH₂CH₃ | Et | H | H | Me | Me | H | H |
| CH₂C═CH₂CH₃ | Et | H | H | H | H | Me | Me |
| CH₂C═CH₂CH₃ | i-Pr | Me | Me | H | H | H | H |
| CH₂C═CH₂CH₃ | i-Pr | H | H | Me | Me | H | H |
| CH₂C═CH₂CH₃ | i-Pr | H | H | H | H | Me | Me |
| CH₂C═CH₂CH₃ | Pr | Me | Me | H | H | H | H |
| CH₂C═CH₂CH₃ | Pr | H | H | Me | Me | H | H |
| CH₂C═CH₂CH₃ | Pr | H | H | H | H | Me | Me |
| CH₂C═CH₂CH₃ | Bu | Me | Me | H | H | H | H |
| CH₂C═CH₂CH₃ | Bu | H | H | Me | Me | H | H |
| CH₂C═CH₂CH₃ | Bu | H | H | H | H | Me | Me |
| CH₂C═CCH₂CH₂CH₃ | H | Me | Me | H | H | H | H |
| CH₂C═CCH₂CH₂CH₃ | H | H | H | Me | Me | H | H |
| CH₂C═CCH₂CH₂CH₃ | H | H | H | H | H | Me | Me |
| CH₂C═CCH₂CH₂CH₃ | Me | Me | Me | H | H | H | H |
| CH₂C═CCH₂CH₂CH₃ | Me | H | H | Me | Me | H | H |
| CH₂C═CCH₂CH₂CH₃ | Me | H | H | H | H | Me | Me |
| CH₂C═CCH₂CH₂CH₃ | Et | Me | Me | H | H | H | H |
| CH₂C═CCH₂CH₂CH₃ | Et | H | H | Me | Me | H | H |
| CH₂C═CCH₂CH₂CH₃ | Et | H | H | H | H | Me | Me |
| CH₂C═CCH₂CH₂CH₃ | i-Pr | Me | Me | H | H | H | H |
| CH₂C═CCH₂CH₂CH₃ | i-Pr | H | H | Me | Me | H | H |
| CH₂C═CCH₂CH₂CH₃ | i-Pr | H | H | H | H | Me | Me |
| CH₂C═CCH₂CH₂CH₃ | Pr | Me | Me | H | H | H | H |
| CH₂C═CCH₂CH₂CH₃ | Pr | H | H | Me | Me | H | H |
| CH₂C═CCH₂CH₂CH₃ | Pr | H | H | H | H | Me | Me |
| CH₂C═CCH₂CH₂CH₃ | Bu | Me | Me | H | H | H | H |
| CH₂C═CCH₂CH₂CH₃ | Bu | H | H | Me | Me | H | H |
| CH₂C═CCH₂CH₂CH₃ | Bu | H | H | H | H | Me | Me |

TABLE L39

| R¹ | R² | R³ | R⁴ | R⁵ | R⁶ | R⁷ | R⁸ |
|---|---|---|---|---|---|---|---|
| CH₂c-Pr | H | Me | Me | H | H | H | H |
| CH₂c-Pr | H | H | H | Me | Me | H | H |
| CH₂c-Pr | H | H | H | H | H | Me | Me |
| CH₂c-Pr | Me | Me | Me | H | H | H | H |
| CH₂c-Pr | Me | H | H | Me | Me | H | H |
| CH₂c-Pr | Me | H | H | H | H | Me | Me |
| CH₂c-Pr | Et | Me | Me | H | H | H | H |
| CH₂c-Pr | Et | H | H | Me | Me | H | H |
| CH₂c-Pr | Et | | H | H | H | Me | Me |
| CH₂c-Pr | i-Pr | Me | Me | H | H | H | H |
| CH₂c-Pr | i-Pr | H | H | Me | Me | H | H |
| CH₂c-Pr | i-Pr | H | H | H | H | Me | Me |
| CH₂c-Pr | Pr | Me | Me | H | H | H | H |
| CH₂c-Pr | Pr | H | H | Me | Me | H | H |
| CH₂c-Pr | Pr | H | H | H | H | Me | Me |
| CH₂c-Pr | Bu | Me | Me | H | H | H | H |
| CH₂c-Pr | Bu | H | H | Me | Me | H | H |
| CH₂c-Pr | Bu | H | H | H | H | Me | Me |

TABLE L40

| R¹ | R² | R³ | R⁴ | R⁵ | R⁶ | R⁷ | R⁸ |
|---|---|---|---|---|---|---|---|
| CH₂-1-CN-c-Pr | H | Me | Me | H | H | H | H |
| CH₂-1-CN-c-Pr | H | H | H | Me | Me | H | H |
| CH₂-1-CN-c-Pr | H | H | H | H | H | Me | Me |
| CH₂-1-CN-c-Pr | Me | Me | Me | H | H | H | H |
| CH₂-1-CN-c-Pr | Me | H | H | Me | Me | H | H |
| CH₂-1-CN-c-Pr | Me | H | H | H | H | Me | Me |
| CH₂-1-CN-c-Pr | Et | Me | Me | H | H | H | H |
| CH₂-1-CN-c-Pr | Et | H | H | Me | Me | H | H |
| CH₂-1-CN-c-Pr | Et | H | H | H | H | Me | Me |
| CH₂-1-CN-c-Pr | i-Pr | Me | Me | H | H | H | H |
| CH₂-1-CN-c-Pr | i-Pr | H | H | Me | Me | H | H |
| CH₂-1-CN-c-Pr | i-Pr | H | H | H | H | Me | Me |
| CH₂-1-CN-c-Pr | Pr | Me | Me | H | H | H | H |
| CH₂-1-CN-c-Pr | Pr | H | H | Me | Me | H | H |
| CH₂-1-CN-c-Pr | Pr | H | H | H | H | Me | Me |
| CH₂-1-CN-c-Pr | Bu | Me | Me | H | H | H | H |
| CH₂-1-CN-c-Pr | Bu | H | H | Me | Me | H | H |
| CH₂-1-CN-c-Pr | Bu | H | H | H | H | Me | Me |

TABLE L41

| R¹ | R² | R³ | R⁴ | R⁵ | R⁶ | R⁷ | R⁸ |
|---|---|---|---|---|---|---|---|
| CH₂-1-F-c-Pr | H | Me | Me | H | H | H | H |
| CH₂-1-F-c-Pr | H | H | H | Me | Me | H | H |
| CH₂-1-F-c-Pr | H | H | H | H | H | Me | Me |
| CH₂-1-F-c-Pr | Me | Me | Me | H | H | H | H |
| CH₂-1-F-c-Pr | Me | H | H | Me | Me | H | H |
| CH₂-1-F-c-Pr | Me | H | H | H | H | Me | Me |
| CH₂-1-F-c-Pr | Et | Me | Me | H | H | H | H |
| CH₂-1-F-c-Pr | Et | H | H | Me | Me | H | H |
| CH₂-1-F-c-Pr | Et | H | H | H | H | Me | Me |
| CH₂-1-F-c-Pr | i-Pr | Me | Me | H | H | H | H |
| CH₂-1-F-c-Pr | i-Pr | H | H | Me | Me | H | H |
| CH₂-1-F-c-Pr | i-Pr | H | H | H | H | Me | Me |
| CH₂-1-F-c-Pr | Pr | Me | Me | H | H | H | H |
| CH₂-1-F-c-Pr | Pr | H | H | Me | Me | H | H |
| CH₂-1-F-c-Pr | Pr | H | H | H | H | Me | Me |
| CH₂-1-F-c-Pr | Bu | Me | Me | H | H | H | H |
| CH₂-1-F-c-Pr | Bu | H | H | Me | Me | H | H |
| CH₂-1-F-c-Pr | Bu | H | H | H | H | Me | Me |

TABLE L42

| R¹ | R² | R³ | R⁴ | R⁵ | R⁶ | R⁷ | R⁸ |
|---|---|---|---|---|---|---|---|
| CH₂c-Bu | H | Me | Me | H | H | H | H |
| CH₂c-Bu | H | H | H | Me | Me | H | H |
| CH₂c-Bu | H | H | H | H | H | Me | Me |
| CH₂c-Bu | Me | Me | Me | H | H | H | H |
| CH₂c-Bu | Me | H | H | Me | Me | H | H |
| CH₂c-Bu | Me | H | H | H | H | Me | Me |
| CH₂c-Bu | Et | Me | Me | H | H | H | H |
| CH₂c-Bu | Et | H | H | Me | Me | H | H |
| CH₂c-Bu | Et | H | H | H | H | Me | Me |
| CH₂c-Bu | i-Pr | Me | Me | H | H | H | H |
| CH₂c-Bu | i-Pr | H | H | Me | Me | H | H |
| CH₂c-Bu | i-Pr | H | H | H | H | Me | Me |
| CH₂c-Bu | Pr | Me | Me | H | H | H | H |
| CH₂c-Bu | Pr | H | H | Me | Me | H | H |
| CH₂c-Bu | Pr | H | H | H | H | Me | Me |
| CH₂c-Bu | Bu | Me | Me | H | H | H | H |
| CH₂c-Bu | Bu | H | H | Me | Me | H | H |
| CH₂c-Bu | Bu | H | H | H | H | Me | Me |

TABLE L43

| R¹ | R² | R³ | R⁴ | R⁵ | R⁶ | R⁷ | R⁸ |
|---|---|---|---|---|---|---|---|
| CH₂c-C₅H₉ | H | Me | Me | H | H | H | H |
| CH₂c-C₅H₉ | H | H | H | Me | Me | H | H |
| CH₂c-C₅H₉ | H | H | H | H | H | Me | Me |
| CH₂c-C₅H₉ | Me | Me | Me | H | H | H | H |
| CH₂c-C₅H₉ | Me | H | H | Me | Me | H | H |
| CH₂c-C₅H₉ | Me | H | H | H | H | Me | Me |
| CH₂c-C₅H₉ | Et | Me | Me | H | H | H | H |
| CH₂c-C₅H₉ | Et | H | H | Me | Me | H | H |
| CH₂c-C₅H₉ | Et | H | H | H | H | Me | Me |
| CH₂c-C₅H₉ | i-Pr | Me | Me | H | H | H | H |
| CH₂c-C₅H₉ | i-Pr | H | H | Me | Me | H | H |
| CH₂c-C₅H₉ | i-Pr | H | H | H | H | Me | Me |
| CH₂c-C₅H₉ | Pr | Me | Me | H | H | H | H |
| CH₂c-C₅H₉ | Pr | H | H | Me | Me | H | H |
| CH₂c-C₅H₉ | Pr | H | H | H | H | Me | Me |
| CH₂c-C₅H₉ | Bu | Me | Me | H | H | H | H |
| CH₂c-C₅H₉ | Bu | H | H | Me | Me | H | H |
| CH₂c-C₅H₉ | Bu | H | H | H | H | Me | Me |

TABLE L44

| R¹ | R² | R³ | R⁴ | R⁵ | R⁶ | R⁷ | R⁸ |
|---|---|---|---|---|---|---|---|
| CH₂-1-cyclopentenyl | H | Me | Me | H | H | H | H |
| CH₂-1-cyclopentenyl | H | H | H | Me | Me | H | H |
| CH₂-1-cyclopentenyl | H | H | H | H | H | Me | Me |
| CH₂-1-cyclopentenyl | Me | Me | Me | H | H | H | H |
| CH₂-1-cyclopentenyl | Me | H | H | Me | Me | H | H |
| CH₂-1-cyclopentenyl | Me | H | H | H | H | Me | Me |
| CH₂-1-cyclopentenyl | Et | Me | Me | H | H | H | H |
| CH₂-1-cyclopentenyl | Et | H | H | Me | Me | H | H |
| CH₂-1-cyclopentenyl | Et | H | H | H | H | Me | Me |
| CH₂-1-cyclopentenyl | i-Pr | Me | Me | H | H | H | H |
| CH₂-1-cyclopentenyl | i-Pr | H | H | Me | Me | H | H |
| CH₂-1-cyclopentenyl | i-Pr | H | H | H | H | Me | Me |
| CH₂-1-cyclopentenyl | Pr | Me | Me | H | H | H | H |
| CH₂-1-cyclopentenyl | Pr | H | H | Me | Me | H | H |
| CH₂-1-cyclopentenyl | Pr | H | H | H | H | Me | Me |
| CH₂-1-cyclopentenyl | Bu | Me | Me | H | H | H | H |
| CH₂-1-cyclopentenyl | Bu | H | H | Me | Me | H | H |
| CH₂-1-cyclopentenyl | Bu | H | H | H | H | Me | Me |

TABLE L45

| R¹ | R² | R³ | R⁴ | R⁵ | R⁶ | R⁷ | R⁸ |
|---|---|---|---|---|---|---|---|
| CH₂c-C₆H₁₁ | H | Me | Me | H | H | H | H |
| CH₂c-C₆H₁₁ | H | H | H | Me | Me | H | H |
| CH₂c-C₆H₁₁ | H | H | H | H | H | Me | Me |
| CH₂c-C₆H₁₁ | Me | Me | Me | H | H | H | H |
| CH₂c-C₆H₁₁ | Me | H | H | Me | Me | H | H |
| CH₂c-C₆H₁₁ | Me | H | H | H | H | Me | Me |
| CH₂c-C₆H₁₁ | Et | Me | Me | H | H | H | H |
| CH₂c-C₆H₁₁ | Et | H | H | Me | Me | H | H |
| CH₂c-C₆H₁₁ | Et | H | H | H | H | Me | Me |
| CH₂c-C₆H₁₁ | i-Pr | Me | Me | H | H | H | H |
| CH₂c-C₆H₁₁ | i-Pr | H | H | Me | Me | H | H |
| CH₂c-C₆H₁₁ | i-Pr | H | H | H | H | Me | Me |
| CH₂c-C₆H₁₁ | Pr | Me | Me | H | H | H | H |
| CH₂c-C₆H₁₁ | Pr | H | H | Me | Me | H | H |
| CH₂c-C₆H₁₁ | Pr | H | H | H | H | Me | Me |
| CH₂c-C₆H₁₁ | Bu | Me | Me | H | H | H | H |
| CH₂c-C₆H₁₁ | Bu | H | H | Me | Me | H | H |
| CH₂c-C₆H₁₁ | Bu | H | H | H | H | Me | Me |

TABLE L46

| R¹ | R² | R³ | R⁴ | R⁵ | R⁶ | R⁷ | R⁸ |
|---|---|---|---|---|---|---|---|
| CH₂-1-cyclohexenyl | H | Me | Me | H | H | H | H |
| CH₂-1-cyclohexenyl | H | H | H | Me | Me | H | H |
| CH₂-1-cyclohexenyl | H | H | H | H | H | Me | Me |
| CH₂-1-cyclohexenyl | Me | Me | Me | H | H | H | H |
| CH₂-1-cyclohexenyl | Me | H | H | Me | Me | H | H |
| CH₂-1-cyclohexenyl | Me | H | H | H | H | Me | Me |
| CH₂-1-cyclohexenyl | Et | Me | Me | H | H | H | H |
| CH₂-1-cyclohexenyl | Et | H | H | Me | Me | H | H |
| CH₂-1-cyclohexenyl | Et | H | H | H | H | Me | Me |
| CH₂-1-cyclohexenyl | i-Pr | Me | Me | H | H | H | H |
| CH₂-1-cyclohexenyl | i-Pr | H | H | Me | Me | H | H |
| CH₂-1-cyclohexenyl | i-Pr | H | H | H | H | Me | Me |
| CH₂-1-cyclohexenyl | Pr | Me | Me | H | H | H | H |
| CH₂-1-cyclohexenyl | Pr | H | H | Me | Me | H | H |
| CH₂-1-cyclohexenyl | Pr | H | H | H | H | Me | Me |
| CH₂-1-cyclohexenyl | Bu | Me | Me | H | H | H | H |
| CH₂-1-cyclohexenyl | Bu | H | H | Me | Me | H | H |
| CH₂-1-cyclohexenyl | Bu | H | H | H | H | Me | Me |

TABLE L47

| R¹ | R² | R³ | R⁴ | R⁵ | R⁶ | R⁷ | R⁸ |
|---|---|---|---|---|---|---|---|
| CH₂CH₂OCH₃ | H | Me | Me | H | H | H | H |
| CH₂CH₂OCH₃ | H | H | H | Me | Me | H | H |
| CH₂CH₂OCH₃ | H | H | H | H | H | Me | Me |
| CH₂CH₂OCH₃ | Me | Me | Me | H | H | H | H |
| CH₂CH₂OCH₃ | Me | H | H | Me | Me | H | H |
| CH₂CH₂OCH₃ | Me | H | H | H | H | Me | Me |
| CH₂CH₂OCH₃ | Et | Me | Me | H | H | H | H |
| CH₂CH₂OCH₃ | Et | H | H | Me | Me | H | H |
| CH₂CH₂OCH₃ | Et | H | H | H | H | Me | Me |
| CH₂CH₂OCH₃ | i-Pr | Me | Me | H | H | H | H |
| CH₂CH₂OCH₃ | i-Pr | H | H | Me | Me | H | H |
| CH₂CH₂OCH₃ | i-Pr | H | H | H | H | Me | Me |
| CH₂CH₂OCH₃ | Pr | Me | Me | H | H | H | H |
| CH₂CH₂OCH₃ | Pr | H | H | Me | Me | H | H |
| CH₂CH₂OCH₃ | Pr | H | H | H | H | Me | Me |
| CH₂CH₂OCH₃ | Bu | Me | Me | H | H | H | H |
| CH₂CH₂OCH₃ | Bu | H | H | Me | Me | H | H |
| CH₂CH₂OCH₃ | Bu | H | H | H | H | Me | Me |
| CH₂CH₂CH₂OCH₃ | H | Me | Me | H | H | H | H |
| CH₂CH₂CH₂OCH₃ | H | H | H | Me | Me | H | H |
| CH₂CH₂CH₂OCH₃ | H | H | H | H | H | Me | Me |
| CH₂CH₂CH₂OCH₃ | Me | Me | Me | H | H | H | H |
| CH₂CH₂CH₂OCH₃ | Me | H | H | Me | Me | H | H |
| CH₂CH₂CH₂OCH₃ | Me | H | H | H | H | Me | Me |
| CH₂CH₂CH₂OCH₃ | Et | Me | Me | H | H | H | H |
| CH₂CH₂CH₂OCH₃ | Et | H | H | Me | Me | H | H |
| CH₂CH₂CH₂OCH₃ | Et | H | H | H | H | Me | Me |
| CH₂CH₂CH₂OCH₃ | i-Pr | Me | Me | H | H | H | H |
| CH₂CH₂CH₂OCH₃ | i-Pr | H | H | Me | Me | H | H |
| CH₂CH₂CH₂OCH₃ | i-Pr | H | H | H | H | Me | Me |
| CH₂CH₂CH₂OCH₃ | Pr | Me | Me | H | H | H | H |
| CH₂CH₂CH₂OCH₃ | Pr | H | H | Me | Me | H | H |
| CH₂CH₂CH₂OCH₃ | Pr | H | H | H | H | Me | Me |
| CH₂CH₂CH₂OCH₃ | Bu | Me | Me | H | H | H | H |
| CH₂CH₂CH₂OCH₃ | Bu | H | H | Me | Me | H | H |
| CH₂CH₂CH₂OCH₃ | Bu | H | H | H | H | Me | Me |

TABLE L48

| R¹ | R² | R³ | R⁴ | R⁵ | R⁶ | R⁷ | R⁸ |
|---|---|---|---|---|---|---|---|
| CH₂CH₂OCH₂CH₃ | H | Me | Me | H | H | H | H |
| CH₂CH₂OCH₂CH₃ | H | H | H | Me | Me | H | H |
| CH₂CH₂OCH₂CH₃ | H | H | H | H | H | Me | Me |
| CH₂CH₂OCH₂CH₃ | Me | Me | Me | H | H | H | H |
| CH₂CH₂OCH₂CH₃ | Me | H | H | Me | Me | H | H |
| CH₂CH₂OCH₂CH₃ | Me | H | H | H | H | Me | Me |
| CH₂CH₂OCH₂CH₃ | Et | Me | Me | H | H | H | H |
| CH₂CH₂OCH₂CH₃ | Et | H | H | Me | Me | H | H |
| CH₂CH₂OCH₂CH₃ | Et | H | H | H | H | Me | Me |
| CH₂CH₂OCH₂CH₃ | i-Pr | Me | Me | H | H | H | H |
| CH₂CH₂OCH₂CH₃ | i-Pr | H | H | Me | Me | H | H |
| CH₂CH₂OCH₂CH₃ | i-Pr | H | H | H | H | Me | Me |
| CH₂CH₂OCH₂CH₃ | Pr | Me | Me | H | H | H | H |
| CH₂CH₂OCH₂CH₃ | Pr | H | H | Me | Me | H | H |
| CH₂CH₂OCH₂CH₃ | Pr | H | H | H | H | Me | Me |
| CH₂CH₂OCH₂CH₃ | Bu | Me | Me | H | H | H | H |
| CH₂CH₂OCH₂CH₃ | Bu | H | H | Me | Me | H | H |
| CH₂CH₂OCH₂CH₃ | Bu | H | H | H | H | Me | Me |
| CH₂CH₂CH₂OCH₂CH₃ | H | Me | Me | H | H | H | H |
| CH₂CH₂CH₂OCH₂CH₃ | H | H | H | Me | Me | H | H |
| CH₂CH₂CH₂OCH₂CH₃ | H | H | H | H | H | Me | Me |
| CH₂CH₂CH₂OCH₂CH₃ | Me | Me | Me | H | H | H | H |
| CH₂CH₂CH₂OCH₂CH₃ | Me | H | H | Me | Me | H | H |
| CH₂CH₂CH₂OCH₂CH₃ | Me | H | H | H | H | Me | Me |
| CH₂CH₂CH₂OCH₂CH₃ | Et | Me | Me | H | H | H | H |
| CH₂CH₂CH₂OCH₂CH₃ | Et | H | H | Me | Me | H | H |
| CH₂CH₂CH₂OCH₂CH₃ | Et | H | H | H | H | Me | Me |
| CH₂CH₂CH₂OCH₂CH₃ | i-Pr | Me | Me | H | H | H | H |
| CH₂CH₂CH₂OCH₂CH₃ | i-Pr | H | H | Me | Me | H | H |
| CH₂CH₂CH₂OCH₂CH₃ | i-Pr | H | H | H | H | Me | Me |
| CH₂CH₂CH₂OCH₂CH₃ | Pr | Me | Me | H | H | H | H |
| CH₂CH₂CH₂OCH₂CH₃ | Pr | H | H | Me | Me | H | H |
| CH₂CH₂CH₂OCH₂CH₃ | Pr | H | H | H | H | Me | Me |
| CH₂CH₂CH₂OCH₂CH₃ | Bu | Me | Me | H | H | H | H |
| CH₂CH₂CH₂OCH₂CH₃ | Bu | H | H | Me | Me | H | H |
| CH₂CH₂CH₂OCH₂CH₃ | Bu | H | H | H | H | Me | Me |

TABLE L49

| R¹ | R² | R³ | R⁴ | R⁵ | R⁶ | R⁷ | R⁸ |
|---|---|---|---|---|---|---|---|
| CH₂CH₂OCH₂CH₂CH₃ | H | Me | Me | H | H | H | H |
| CH₂CH₂OCH₂CH₂CH₃ | H | H | H | Me | Me | H | H |
| CH₂CH₂OCH₂CH₂CH₃ | H | H | H | H | H | Me | Me |
| CH₂CH₂OCH₂CH₂CH₃ | Me | Me | Me | H | H | H | H |
| CH₂CH₂OCH₂CH₂CH₃ | Me | H | H | Me | Me | H | H |
| CH₂CH₂OCH₂CH₂CH₃ | Me | H | H | H | H | Me | Me |
| CH₂CH₂OCH₂CH₂CH₃ | Et | Me | Me | H | H | H | H |
| CH₂CH₂OCH₂CH₂CH₃ | Et | H | H | Me | Me | H | H |
| CH₂CH₂OCH₂CH₂CH₃ | Et | H | H | H | H | Me | Me |
| CH₂CH₂OCH₂CH₂CH₃ | i-Pr | Me | Me | H | H | H | H |
| CH₂CH₂OCH₂CH₂CH₃ | i-Pr | H | H | Me | Me | H | H |
| CH₂CH₂OCH₂CH₂CH₃ | i-Pr | H | H | H | H | Me | Me |
| CH₂CH₂OCH₂CH₂CH₃ | Pr | Me | Me | H | H | H | H |
| CH₂CH₂OCH₂CH₂CH₃ | Pr | H | H | Me | Me | H | H |
| CH₂CH₂OCH₂CH₂CH₃ | Pr | H | H | H | H | Me | Me |
| CH₂CH₂OCH₂CH₂CH₃ | Bu | Me | Me | H | H | H | H |
| CH₂CH₂OCH₂CH₂CH₃ | Bu | H | H | Me | Me | H | H |
| CH₂CH₂OCH₂CH₂CH₃ | Bu | H | H | H | H | Me | Me |
| CH₂CH₂CH₂CH₂OCH₃ | H | Me | Me | H | H | H | H |
| CH₂CH₂CH₂CH₂OCH₃ | H | H | H | Me | Me | H | H |
| CH₂CH₂CH₂CH₂OCH₃ | H | H | H | H | H | Me | Me |
| CH₂CH₂CH₂CH₂OCH₃ | Me | Me | Me | H | H | H | H |
| CH₂CH₂CH₂CH₂OCH₃ | Me | H | H | Me | Me | H | H |
| CH₂CH₂CH₂CH₂OCH₃ | Me | H | H | H | H | Me | Me |
| CH₂CH₂CH₂CH₂OCH₃ | Et | Me | Me | H | H | H | H |
| CH₂CH₂CH₂CH₂OCH₃ | Et | H | H | Me | Me | H | H |
| CH₂CH₂CH₂CH₂OCH₃ | Et | H | H | H | H | Me | Me |
| CH₂CH₂CH₂CH₂OCH₃ | i-Pr | Me | Me | H | H | H | H |
| CH₂CH₂CH₂CH₂OCH₃ | i-Pr | H | H | Me | Me | H | H |
| CH₂CH₂CH₂CH₂OCH₃ | i-Pr | H | H | H | H | Me | Me |
| CH₂CH₂CH₂CH₂OCH₃ | Pr | Me | Me | H | H | H | H |
| CH₂CH₂CH₂CH₂OCH₃ | Pr | H | H | Me | Me | H | H |
| CH₂CH₂CH₂CH₂OCH₃ | Pr | H | H | H | H | Me | Me |
| CH₂CH₂CH₂CH₂OCH₃ | Bu | Me | Me | H | H | H | H |
| CH₂CH₂CH₂CH₂OCH₃ | Bu | H | H | Me | Me | H | H |
| CH₂CH₂CH₂CH₂OCH₃ | Bu | H | H | H | H | Me | Me |

TABLE L50

| R¹ | R² | R³ | R⁴ | R⁵ | R⁶ | R⁷ | R⁸ |
|---|---|---|---|---|---|---|---|
| CH₂C(O)CH₃ | H | Me | Me | H | H | H | H |
| CH₂C(O)CH₃ | H | H | H | Me | Me | H | H |
| CH₂C(O)CH₃ | H | H | H | H | H | Me | Me |
| CH₂C(O)CH₃ | Me | Me | Me | H | H | H | H |
| CH₂C(O)CH₃ | Me | H | H | Me | Me | H | H |
| CH₂C(O)CH₃ | Me | H | H | H | H | Me | Me |
| CH₂C(O)CH₃ | Et | Me | Me | H | H | H | H |
| CH₂C(O)CH₃ | Et | H | H | Me | Me | H | H |
| CH₂C(O)CH₃ | Et | H | H | H | H | Me | Me |
| CH₂C(O)CH₃ | i-Pr | Me | Me | H | H | H | H |
| CH₂C(O)CH₃ | i-Pr | H | H | Me | Me | H | H |
| CH₂C(O)CH₃ | i-Pr | H | H | H | H | Me | Me |
| CH₂C(O)CH₃ | Pr | Me | Me | H | H | H | H |
| CH₂C(O)CH₃ | Pr | H | H | Me | Me | H | H |
| CH₂C(O)CH₃ | Pr | H | H | H | H | Me | Me |
| CH₂C(O)CH₃ | Bu | Me | Me | H | H | H | H |
| CH₂C(O)CH₃ | Bu | H | H | Me | Me | H | H |
| CH₂C(O)CH₃ | Bu | H | H | H | H | Me | Me |
| CH₂C(O)CH₂CH₃ | H | Me | Me | H | H | H | H |
| CH₂C(O)CH₂CH₃ | H | H | H | Me | Me | H | H |
| CH₂C(O)CH₂CH₃ | H | H | H | H | H | Me | Me |
| CH₂C(O)CH₂CH₃ | Me | Me | Me | H | H | H | H |
| CH₂C(O)CH₂CH₃ | Me | H | H | Me | Me | H | H |
| CH₂C(O)CH₂CH₃ | Me | H | H | H | H | Me | Me |
| CH₂C(O)CH₂CH₃ | Et | Me | Me | H | H | H | H |
| CH₂C(O)CH₂CH₃ | Et | H | H | Me | Me | H | H |
| CH₂C(O)CH₂CH₃ | Et | H | H | H | H | Me | Me |
| CH₂C(O)CH₂CH₃ | i-Pr | Me | Me | H | H | H | H |
| CH₂C(O)CH₂CH₃ | i-Pr | H | H | Me | Me | H | H |
| CH₂C(O)CH₂CH₃ | i-Pr | H | H | H | H | Me | Me |
| CH₂C(O)CH₂CH₃ | Pr | Me | Me | H | H | H | H |
| CH₂C(O)CH₂CH₃ | Pr | H | H | Me | Me | H | H |
| CH₂C(O)CH₂CH₃ | Pr | H | H | H | H | Me | Me |
| CH₂C(O)CH₂CH₃ | Bu | Me | Me | H | H | H | H |
| CH₂C(O)CH₂CH₃ | Bu | H | H | Me | Me | H | H |
| CH₂C(O)CH₂CH₃ | Bu | H | H | H | H | Me | Me |

TABLE L51

| R¹ | R² | R³ | R⁴ | R⁵ | R⁶ | R⁷ | R⁸ |
|---|---|---|---|---|---|---|---|
| CH₂C(O)CF₃ | H | Me | Me | H | H | H | H |
| CH₂C(O)CF₃ | H | H | H | Me | Me | H | H |
| CH₂C(O)CF₃ | H | H | H | H | H | Me | Me |
| CH₂C(O)CF₃ | Me | Me | Me | H | H | H | H |
| CH₂C(O)CF₃ | Me | H | H | Me | Me | H | H |
| CH₂C(O)CF₃ | Me | H | H | H | H | Me | Me |
| CH₂C(O)CF₃ | Et | Me | Me | H | H | H | H |
| CH₂C(O)CF₃ | Et | H | H | Me | Me | H | H |
| CH₂C(O)CF₃ | Et | H | H | H | H | Me | Me |
| CH₂C(O)CF₃ | i-Pr | Me | Me | H | H | H | H |
| CH₂C(O)CF₃ | i-Pr | H | H | Me | Me | H | H |
| CH₂C(O)CF₃ | i-Pr | H | H | H | H | Me | Me |
| CH₂C(O)CF₃ | Pr | Me | Me | H | H | H | H |
| CH₂C(O)CF₃ | Pr | H | H | Me | Me | H | H |
| CH₂C(O)CF₃ | Pr | H | H | H | H | Me | Me |
| CH₂C(O)CF₃ | Bu | Me | Me | H | H | H | H |
| CH₂C(O)CF₃ | Bu | H | H | Me | Me | H | H |
| CH₂C(O)CF₃ | Bu | H | H | H | H | Me | Me |
| CH₂C(O)OCH₃ | H | Me | Me | H | H | H | H |
| CH₂C(O)OCH₃ | H | H | H | Me | Me | H | H |
| CH₂C(O)OCH₃ | H | H | H | H | H | Me | Me |
| CH₂C(O)OCH₃ | Me | Me | Me | H | H | H | H |
| CH₂C(O)OCH₃ | Me | H | H | Me | Me | H | H |
| CH₂C(O)OCH₃ | Me | H | H | H | H | Me | Me |
| CH₂C(O)OCH₃ | Et | Me | Me | H | H | H | H |
| CH₂C(O)OCH₃ | Et | H | H | Me | Me | H | H |
| CH₂C(O)OCH₃ | Et | H | H | H | H | Me | Me |
| CH₂C(O)OCH₃ | i-Pr | Me | Me | H | H | H | H |
| CH₂C(O)OCH₃ | i-Pr | H | H | Me | Me | H | H |
| CH₂C(O)OCH₃ | i-Pr | H | H | H | H | Me | Me |
| CH₂C(O)OCH₃ | Pr | Me | Me | H | H | H | H |
| CH₂C(O)OCH₃ | Pr | H | H | Me | Me | H | H |
| CH₂C(O)OCH₃ | Pr | H | H | H | H | Me | Me |
| CH₂C(O)OCH₃ | Bu | Me | Me | H | H | H | H |
| CH₂C(O)OCH₃ | Bu | H | H | Me | Me | H | H |
| CH₂C(O)OCH₃ | Bu | H | H | H | H | Me | Me |

TABLE L52

| R¹ | R² | R³ | R⁴ | R⁵ | R⁶ | R⁷ | R⁸ |
|---|---|---|---|---|---|---|---|
| CH₂C(O)OCH₂CH₃ | H | Me | Me | H | H | H | H |
| CH₂C(O)OCH₂CH₃ | H | H | H | Me | Me | H | H |
| CH₂C(O)OCH₂CH₃ | H | H | H | H | H | Me | Me |
| CH₂C(O)OCH₂CH₃ | Me | Me | Me | H | H | H | H |
| CH₂C(O)OCH₂CH₃ | Me | H | H | Me | Me | H | H |
| CH₂C(O)OCH₂CH₃ | Me | H | H | H | H | Me | Me |
| CH₂C(O)OCH₂CH₃ | Et | Me | Me | H | H | H | H |
| CH₂C(O)OCH₂CH₃ | Et | H | H | Me | Me | H | H |
| CH₂C(O)OCH₂CH₃ | Et | H | H | H | H | Me | Me |
| CH₂C(O)OCH₂CH₃ | i-Pr | Me | Me | H | H | H | H |
| CH₂C(O)OCH₂CH₃ | i-Pr | H | H | Me | Me | H | H |
| CH₂C(O)OCH₂CH₃ | i-Pr | H | H | H | H | Me | Me |
| CH₂C(O)OCH₂CH₃ | Pr | Me | Me | H | H | H | H |
| CH₂C(O)OCH₂CH₃ | Pr | H | H | Me | Me | H | H |
| CH₂C(O)OCH₂CH₃ | Pr | H | H | H | H | Me | Me |
| CH₂C(O)OCH₂CH₃ | Bu | Me | Me | H | H | H | H |
| CH₂C(O)OCH₂CH₃ | Bu | H | H | Me | Me | H | H |
| CH₂C(O)OCH₂CH₃ | Bu | H | H | H | H | Me | Me |
| CH₂C(O)OCF₃ | H | Me | Me | H | H | H | H |
| CH₂C(O)OCF₃ | H | H | H | Me | Me | H | H |
| CH₂C(O)OCF₃ | H | H | H | H | H | Me | Me |
| CH₂C(O)OCF₃ | Me | Me | Me | H | H | H | H |
| CH₂C(O)OCF₃ | Me | H | H | Me | Me | H | H |
| CH₂C(O)OCF₃ | Me | H | H | H | H | Me | Me |
| CH₂C(O)OCF₃ | Et | Me | Me | H | H | H | H |
| CH₂C(O)OCF₃ | Et | H | H | Me | Me | H | H |
| CH₂C(O)OCF₃ | Et | H | H | H | H | Me | Me |
| CH₂C(O)OCF₃ | i-Pr | Me | Me | H | H | H | H |
| CH₂C(O)OCF₃ | i-Pr | H | H | Me | Me | H | H |
| CH₂C(O)OCF₃ | i-Pr | H | H | H | H | Me | Me |
| CH₂C(O)OCF₃ | Pr | Me | Me | H | H | H | H |
| CH₂C(O)OCF₃ | Pr | H | H | Me | Me | H | H |
| CH₂C(O)OCF₃ | Pr | H | H | H | H | Me | Me |
| CH₂C(O)OCF₃ | Bu | Me | Me | H | H | H | H |
| CH₂C(O)OCF₃ | Bu | H | H | Me | Me | H | H |
| CH₂C(O)OCF₃ | Bu | H | H | H | H | Me | Me |

TABLE L53

| R¹ | R² | R³ | R⁴ | R⁵ | R⁶ | R⁷ | R⁸ |
|---|---|---|---|---|---|---|---|
| CH₂S(O)₂CH₃ | H | Me | Me | H | H | H | H |
| CH₂S(O)₂CH₃ | H | H | H | Me | Me | H | H |
| CH₂S(O)₂CH₃ | H | H | H | H | H | Me | Me |
| CH₂S(O)₂CH₃ | Me | Me | Me | H | H | H | H |
| CH₂S(O)₂CH₃ | Me | H | H | Me | Me | H | H |
| CH₂S(O)₂CH₃ | Me | H | H | H | H | Me | Me |
| CH₂S(O)₂CH₃ | Et | Me | Me | H | H | H | H |
| CH₂S(O)₂CH₃ | Et | H | H | Me | Me | H | H |
| CH₂S(O)₂CH₃ | Et | H | H | H | H | Me | Me |
| CH₂S(O)₂CH₃ | i-Pr | Me | Me | H | H | H | H |
| CH₂S(O)₂CH₃ | i-Pr | H | H | Me | Me | H | H |
| CH₂S(O)₂CH₃ | i-Pr | H | H | H | H | Me | Me |
| CH₂S(O)₂CH₃ | Pr | Me | Me | H | H | H | H |
| CH₂S(O)₂CH₃ | Pr | H | H | Me | Me | H | H |
| CH₂S(O)₂CH₃ | Pr | H | H | H | H | Me | Me |
| CH₂S(O)₂CH₃ | Bu | Me | Me | H | H | H | H |
| CH₂S(O)₂CH₃ | Bu | H | H | Me | Me | H | H |
| CH₂S(O)₂CH₃ | Bu | H | H | H | H | Me | Me |
| CH₂S(O)₂CH₂CH₃ | H | Me | Me | H | H | H | H |
| CH₂S(O)₂CH₂CH₃ | H | H | H | Me | Me | H | H |
| CH₂S(O)₂CH₂CH₃ | H | H | H | H | H | Me | Me |
| CH₂S(O)₂CH₂CH₃ | Me | Me | Me | H | H | H | H |
| CH₂S(O)₂CH₂CH₃ | Me | H | H | Me | Me | H | H |
| CH₂S(O)₂CH₂CH₃ | Me | H | H | H | H | Me | Me |
| CH₂S(O)₂CH₂CH₃ | Et | Me | Me | H | H | H | H |
| CH₂S(O)₂CH₂CH₃ | Et | H | H | Me | Me | H | H |
| CH₂S(O)₂CH₂CH₃ | Et | H | H | H | H | Me | Me |
| CH₂S(O)₂CH₂CH₃ | i-Pr | Me | Me | H | H | H | H |
| CH₂S(O)₂CH₂CH₃ | i-Pr | H | H | Me | Me | H | H |
| CH₂S(O)₂CH₂CH₃ | i-Pr | H | H | H | H | Me | Me |
| CH₂S(O)₂CH₂CH₃ | Pr | Me | Me | H | H | H | H |
| CH₂S(O)₂CH₂CH₃ | Pr | H | H | Me | Me | H | H |
| CH₂S(O)₂CH₂CH₃ | Pr | H | H | H | H | Me | Me |
| CH₂S(O)₂CH₂CH₃ | Bu | Me | Me | H | H | H | H |
| CH₂S(O)₂CH₂CH₃ | Bu | H | H | Me | Me | H | H |
| CH₂S(O)₂CH₂CH₃ | Bu | H | H | H | H | Me | Me |

TABLE L54

| R¹ | R² | R³ | R⁴ | R⁵ | R⁶ | R⁷ | R⁸ |
|---|---|---|---|---|---|---|---|
| CH₂S(O)₂CF₃ | H | Me | Me | H | H | H | H |
| CH₂S(O)₂CF₃ | H | H | H | Me | Me | H | H |
| CH₂S(O)₂CF₃ | H | H | H | H | H | Me | Me |
| CH₂S(O)₂CF₃ | Me | Me | Me | H | H | H | H |
| CH₂S(O)₂CF₃ | Me | H | H | Me | Me | H | H |
| CH₂S(O)₂CF₃ | Me | H | H | H | H | Me | Me |
| CH₂S(O)₂CF₃ | Et | Me | Me | H | H | H | H |
| CH₂S(O)₂CF₃ | Et | H | H | Me | Me | H | H |
| CH₂S(O)₂CF₃ | Et | H | H | H | H | Me | Me |
| CH₂S(O)₂CF₃ | i-Pr | Me | Me | H | H | H | H |
| CH₂S(O)₂CF₃ | i-Pr | H | H | Me | Me | H | H |
| CH₂S(O)₂CF₃ | i-Pr | H | H | H | H | Me | Me |
| CH₂S(O)₂CF₃ | Pr | Me | Me | H | H | H | H |
| CH₂S(O)₂CF₃ | Pr | H | H | Me | Me | H | H |
| CH₂S(O)₂CF₃ | Pr | H | H | H | H | Me | Me |
| CH₂S(O)₂CF₃ | Bu | Me | Me | H | H | H | H |
| CH₂S(O)₂CF₃ | Bu | H | H | Me | Me | H | H |
| CH₂S(O)₂CF₃ | Bu | H | H | H | H | Me | Me |
| CH₂CF₃ | H | Me | Me | H | H | H | H |
| CH₂CF₃ | H | H | H | Me | Me | H | H |
| CH₂CF₃ | H | H | H | H | H | Me | Me |
| CH₂CF₃ | Me | Me | Me | H | H | H | H |
| CH₂CF₃ | Me | H | H | Me | Me | H | H |
| CH₂CF₃ | Me | H | H | H | H | Me | Me |
| CH₂CF₃ | Et | Me | Me | H | H | H | H |
| CH₂CF₃ | Et | H | H | Me | Me | H | H |
| CH₂CF₃ | Et | H | H | H | H | Me | Me |
| CH₂CF₃ | i-Pr | Me | Me | H | H | H | H |
| CH₂CF₃ | i-Pr | H | H | Me | Me | H | H |
| CH₂CF₃ | i-Pr | H | H | H | H | Me | Me |
| CH₂CF₃ | Pr | Me | Me | H | H | H | H |
| CH₂CF₃ | Pr | H | H | Me | Me | H | H |
| CH₂CF₃ | Pr | H | H | H | H | Me | Me |
| CH₂CF₃ | Bu | Me | Me | H | H | H | H |
| CH₂CF₃ | Bu | H | H | Me | Me | H | H |
| CH₂CF₃ | Bu | H | H | H | H | Me | Me |

TABLE L55

| R¹ | R² | R³ | R⁴ | R⁵ | R⁶ | R⁷ | R⁸ |
|---|---|---|---|---|---|---|---|
| CH₂CF₂CF₃ | H | Me | Me | H | H | H | H |
| CH₂CF₂CF₃ | H | H | H | Me | Me | H | H |
| CH₂CF₂CF₃ | H | H | H | H | H | Me | Me |
| CH₂CF₂CF₃ | Me | Me | Me | H | H | H | H |
| CH₂CF₂CF₃ | Me | H | H | Me | Me | H | H |
| CH₂CF₂CF₃ | Me | H | H | H | H | Me | Me |
| CH₂CF₂CF₃ | Et | Me | Me | H | H | H | H |
| CH₂CF₂CF₃ | Et | H | H | Me | Me | H | H |
| CH₂CF₂CF₃ | Et | H | H | H | H | Me | Me |
| CH₂CF₂CF₃ | i-Pr | Me | Me | H | H | H | H |
| CH₂CF₂CF₃ | i-Pr | H | H | Me | Me | H | H |
| CH₂CF₂CF₃ | i-Pr | H | H | H | H | Me | Me |
| CH₂CF₂CF₃ | Pr | Me | Me | H | H | H | H |
| CH₂CF₂CF₃ | Pr | H | H | Me | Me | H | H |
| CH₂CF₂CF₃ | Pr | H | H | H | H | Me | Me |
| CH₂CF₂CF₃ | Bu | Me | Me | H | H | H | H |
| CH₂CF₂CF₃ | Bu | H | H | Me | Me | H | H |
| CH₂CF₂CF₃ | Bu | H | H | H | H | Me | Me |
| CH₂CH₂CF₃ | H | Me | Me | H | H | H | H |
| CH₂CH₂CF₃ | H | H | H | Me | Me | H | H |
| CH₂CH₂CF₃ | H | H | H | H | H | Me | Me |
| CH₂CH₂CF₃ | Me | Me | Me | H | H | H | H |
| CH₂CH₂CF₃ | Me | H | H | Me | Me | H | H |
| CH₂CH₂CF₃ | Me | H | H | H | H | Me | Me |
| CH₂CH₂CF₃ | Et | Me | Me | H | H | H | H |
| CH₂CH₂CF₃ | Et | H | H | Me | Me | H | H |
| CH₂CH₂CF₃ | Et | H | H | H | H | Me | Me |
| CH₂CH₂CF₃ | i-Pr | Me | Me | H | H | H | H |
| CH₂CH₂CF₃ | i-Pr | H | H | Me | Me | H | H |
| CH₂CH₂CF₃ | i-Pr | H | H | H | H | Me | Me |
| CH₂CH₂CF₃ | Pr | Me | Me | H | H | H | H |
| CH₂CH₂CF₃ | Pr | H | H | Me | Me | H | H |
| CH₂CH₂CF₃ | Pr | H | H | H | H | Me | Me |
| CH₂CH₂CF₃ | Bu | Me | Me | H | H | H | H |
| CH₂CH₂CF₃ | Bu | H | H | Me | Me | H | H |
| CH₂CH₂CF₃ | Bu | H | H | H | H | Me | Me |

TABLE L56

| R¹ | R² | R³ | R⁴ | R⁵ | R⁶ | R⁷ | R⁸ |
|---|---|---|---|---|---|---|---|
| CH₂CH₂CH₂CF₃ | H | Me | Me | H | H | H | H |
| CH₂CH₂CH₂CF₃ | H | H | H | Me | Me | H | H |
| CH₂CH₂CH₂CF₃ | H | H | H | H | H | Me | Me |
| CH₂CH₂CH₂CF₃ | Me | Me | Me | H | H | H | H |
| CH₂CH₂CH₂CF₃ | Me | H | H | Me | Me | H | H |
| CH₂CH₂CH₂CF₃ | Me | H | H | H | H | Me | Me |
| CH₂CH₂CH₂CF₃ | Et | Me | Me | H | H | H | H |
| CH₂CH₂CH₂CF₃ | Et | H | H | Me | Me | H | H |
| CH₂CH₂CH₂CF₃ | Et | H | H | H | H | Me | Me |
| CH₂CH₂CH₂CF₃ | i-Pr | Me | Me | H | H | H | H |
| CH₂CH₂CH₂CF₃ | i-Pr | H | H | Me | Me | H | H |
| CH₂CH₂CH₂CF₃ | i-Pr | H | H | H | H | Me | Me |
| CH₂CH₂CH₂CF₃ | Pr | Me | Me | H | H | H | H |
| CH₂CH₂CH₂CF₃ | Pr | H | H | Me | Me | H | H |
| CH₂CH₂CH₂CF₃ | Pr | H | H | H | H | Me | Me |
| CH₂CH₂CH₂CF₃ | Bu | Me | Me | H | H | H | H |
| CH₂CH₂CH₂CF₃ | Bu | H | H | Me | Me | H | H |
| CH₂CH₂CH₂CF₃ | Bu | H | H | H | H | Me | Me |
| CH₂CH₂CF₂CF₃ | H | Me | Me | H | H | H | H |
| CH₂CH₂CF₂CF₃ | H | H | H | Me | Me | H | H |
| CH₂CH₂CF₂CF₃ | H | H | H | H | H | Me | Me |
| CH₂CH₂CF₂CF₃ | Me | Me | Me | H | H | H | H |
| CH₂CH₂CF₂CF₃ | Me | H | H | Me | Me | H | H |
| CH₂CH₂CF₂CF₃ | Me | H | H | H | H | Me | Me |
| CH₂CH₂CF₂CF₃ | Et | Me | Me | H | H | H | H |
| CH₂CH₂CF₂CF₃ | Et | H | H | Me | Me | H | H |
| CH₂CH₂CF₂CF₃ | Et | H | H | H | H | Me | Me |
| CH₂CH₂CF₂CF₃ | i-Pr | Me | Me | H | H | H | H |
| CH₂CH₂CF₂CF₃ | i-Pr | H | H | Me | Me | H | H |
| CH₂CH₂CF₂CF₃ | i-Pr | H | H | H | H | Me | Me |
| CH₂CH₂CF₂CF₃ | Pr | Me | Me | H | H | H | H |
| CH₂CH₂CF₂CF₃ | Pr | H | H | Me | Me | H | H |
| CH₂CH₂CF₂CF₃ | Pr | H | H | H | H | Me | Me |
| CH₂CH₂CF₂CF₃ | Bu | Me | Me | H | H | H | H |
| CH₂CH₂CF₂CF₃ | Bu | H | H | Me | Me | H | H |
| CH₂CH₂CF₂CF₃ | Bu | H | H | H | H | Me | Me |

TABLE L57

| R¹ | R² | R³ | R⁴ | R⁵ | R⁶ | R⁷ | R⁸ |
|---|---|---|---|---|---|---|---|
| CH₂CCl₃ | H | Me | Me | H | H | H | H |
| CH₂CCl₃ | H | H | H | Me | Me | H | H |
| CH₂CCl₃ | H | H | H | H | H | Me | Me |
| CH₂CCl₃ | Me | Me | Me | H | H | H | H |
| CH₂CCl₃ | Me | H | H | Me | Me | H | H |
| CH₂CCl₃ | Me | H | H | H | H | Me | Me |
| CH₂CCl₃ | Et | Me | Me | H | H | H | H |
| CH₂CCl₃ | Et | H | H | Me | Me | H | H |
| CH₂CCl₃ | Et | H | H | H | H | Me | Me |
| CH₂CCl₃ | i-Pr | Me | Me | H | H | H | H |
| CH₂CCl₃ | i-Pr | H | H | Me | Me | H | H |
| CH₂CCl₃ | i-Pr | H | H | H | H | Me | Me |
| CH₂CCl₃ | Pr | Me | Me | H | H | H | H |
| CH₂CCl₃ | Pr | H | H | Me | Me | H | H |
| CH₂CCl₃ | Pr | H | H | H | H | Me | Me |
| CH₂CCl₃ | Bu | Me | Me | H | H | H | H |
| CH₂CCl₃ | Bu | H | H | Me | Me | H | H |
| CH₂CCl₃ | Bu | H | H | H | H | Me | Me |
| CH₂CBr₃ | H | Me | Me | H | H | H | H |
| CH₂CBr₃ | H | H | H | Me | Me | H | H |
| CH₂CBr₃ | H | H | H | H | H | Me | Me |
| CH₂CBr₃ | Me | Me | Me | H | H | H | H |
| CH₂CBr₃ | Me | H | H | Me | Me | H | H |
| CH₂CBr₃ | Me | H | H | H | H | Me | Me |
| CH₂CBr₃ | Et | Me | Me | H | H | H | H |
| CH₂CBr₃ | Et | H | H | Me | Me | H | H |
| CH₂CBr₃ | Et | H | H | H | H | Me | Me |
| CH₂CBr₃ | i-Pr | Me | Me | H | H | H | H |
| CH₂CBr₃ | i-Pr | H | H | Me | Me | H | H |
| CH₂CBr₃ | i-Pr | H | H | H | H | Me | Me |
| CH₂CBr₃ | Pr | Me | Me | H | H | H | H |
| CH₂CBr₃ | Pr | H | H | Me | Me | H | H |
| CH₂CBr₃ | Pr | H | H | H | H | Me | Me |
| CH₂CBr₃ | Bu | Me | Me | H | H | H | H |
| CH₂CBr₃ | Bu | H | H | Me | Me | H | H |
| CH₂CBr₃ | Bu | H | H | H | H | Me | Me |

TABLE L58

| R¹ | R² | R³ | R⁴ | R⁵ | R⁶ | R⁷ | R⁸ |
|---|---|---|---|---|---|---|---|
| CH₂CH₂OH | H | Me | Me | H | H | H | H |
| CH₂CH₂OH | H | H | H | Me | Me | H | H |
| CH₂CH₂OH | H | H | H | H | H | Me | Me |
| CH₂CH₂OH | Me | Me | Me | H | H | H | H |
| CH₂CH₂OH | Me | H | H | Me | Me | H | H |
| CH₂CH₂OH | Me | H | H | H | H | Me | Me |
| CH₂CH₂OH | Et | Me | Me | H | H | H | H |
| CH₂CH₂OH | Et | H | H | Me | Me | H | H |
| CH₂CH₂OH | Et | H | H | H | H | Me | Me |
| CH₂CH₂OH | i-Pr | Me | Me | H | H | H | H |
| CH₂CH₂OH | i-Pr | H | H | Me | Me | H | H |
| CH₂CH₂OH | i-Pr | H | H | H | H | Me | Me |
| CH₂CH₂OH | Pr | Me | Me | H | H | H | H |
| CH₂CH₂OH | Pr | H | H | Me | Me | H | H |
| CH₂CH₂OH | Pr | H | H | H | H | Me | Me |
| CH₂CH₂OH | Bu | Me | Me | H | H | H | H |
| CH₂CH₂OH | Bu | H | H | Me | Me | H | H |
| CH₂CH₂OH | Bu | H | H | H | H | Me | Me |
| CH₂CH(OH)CH₃ | H | Me | Me | H | H | H | H |
| CH₂CH(OH)CH₃ | H | H | H | Me | Me | H | H |
| CH₂CH(OH)CH₃ | H | H | H | H | H | Me | Me |
| CH₂CH(OH)CH₃ | Me | Me | Me | H | H | H | H |
| CH₂CH(OH)CH₃ | Me | H | H | Me | Me | H | H |
| CH₂CH(OH)CH₃ | Me | H | H | H | H | Me | Me |
| CH₂CH(OH)CH₃ | Et | Me | Me | H | H | H | H |
| CH₂CH(OH)CH₃ | Et | H | H | Me | Me | H | H |
| CH₂CH(OH)CH₃ | Et | H | H | H | H | Me | Me |
| CH₂CH(OH)CH₃ | i-Pr | Me | Me | H | H | H | H |
| CH₂CH(OH)CH₃ | i-Pr | H | H | Me | Me | H | H |
| CH₂CH(OH)CH₃ | i-Pr | H | H | H | H | Me | Me |
| CH₂CH(OH)CH₃ | Pr | Me | Me | H | H | H | H |
| CH₂CH(OH)CH₃ | Pr | H | H | Me | Me | H | H |
| CH₂CH(OH)CH₃ | Pr | H | H | H | H | Me | Me |
| CH₂CH(OH)CH₃ | Bu | Me | Me | H | H | H | H |
| CH₂CH(OH)CH₃ | Bu | H | H | Me | Me | H | H |
| CH₂CH(OH)CH₃ | Bu | H | H | H | H | Me | Me |

TABLE L59

| R¹ | R² | R³ | R⁴ | R⁵ | R⁶ | R⁷ | R⁸ |
|---|---|---|---|---|---|---|---|
| CH₂CH₂CH₂OH | H | Me | Me | H | H | H | H |
| CH₂CH₂CH₂OH | H | H | H | Me | Me | H | H |
| CH₂CH₂CH₂OH | H | H | H | H | H | Me | Me |
| CH₂CH₂CH₂OH | Me | Me | Me | H | H | H | H |
| CH₂CH₂CH₂OH | Me | H | H | Me | Me | H | H |
| CH₂CH₂CH₂OH | Me | H | H | H | H | Me | Me |
| CH₂CH₂CH₂OH | Et | Me | Me | H | H | H | H |
| CH₂CH₂CH₂OH | Et | H | H | Me | Me | H | H |
| CH₂CH₂CH₂OH | Et | H | H | H | H | Me | Me |
| CH₂CH₂CH₂OH | i-Pr | Me | Me | H | H | H | H |
| CH₂CH₂CH₂OH | i-Pr | H | H | Me | Me | H | H |
| CH₂CH₂CH₂OH | i-Pr | H | H | H | H | Me | Me |
| CH₂CH₂CH₂OH | Pr | Me | Me | H | H | H | H |
| CH₂CH₂CH₂OH | Pr | H | H | Me | Me | H | H |
| CH₂CH₂CH₂OH | Pr | H | H | H | H | Me | Me |
| CH₂CH₂CH₂OH | Bu | Me | Me | H | H | H | H |
| CH₂CH₂CH₂OH | Bu | H | H | Me | Me | H | H |
| CH₂CH₂CH₂OH | Bu | H | H | H | H | Me | Me |
| CH₂CN | H | Me | Me | H | H | H | H |
| CH₂CN | H | H | H | Me | Me | H | H |
| CH₂CN | H | H | H | H | H | Me | Me |
| CH₂CN | Me | Me | Me | H | H | H | H |
| CH₂CN | Me | H | H | Me | Me | H | H |
| CH₂CN | Me | H | H | H | H | Me | Me |
| CH₂CN | Et | Me | Me | H | H | H | H |
| CH₂CN | Et | H | H | Me | Me | H | H |
| CH₂CN | Et | H | H | H | H | Me | Me |
| CH₂CN | i-Pr | Me | Me | H | H | H | H |
| CH₂CN | i-Pr | H | H | Me | Me | H | H |
| CH₂CN | i-Pr | H | H | H | H | Me | Me |
| CH₂CN | Pr | Me | Me | H | H | H | H |
| CH₂CN | Pr | H | H | Me | Me | H | H |
| CH₂CN | Pr | H | H | H | H | Me | Me |
| CH₂CN | Bu | Me | Me | H | H | H | H |
| CH₂CN | Bu | H | H | Me | Me | H | H |
| CH₂CN | Bu | H | H | H | H | Me | Me |

TABLE L60

| R¹ | R² | R³ | R⁴ | R⁵ | R⁶ | R⁷ | R⁸ |
|---|---|---|---|---|---|---|---|
| CH(CH₃)CN | H | Me | Me | H | H | H | H |
| CH(CH₃)CN | H | H | H | Me | Me | H | H |
| CH(CH₃)CN | H | H | H | H | H | Me | Me |
| CH(CH₃)CN | Me | Me | Me | H | H | H | H |
| CH(CH₃)CN | Me | H | H | Me | Me | H | H |
| CH(CH₃)CN | Me | H | H | H | H | Me | Me |
| CH(CH₃)CN | Et | Me | Me | H | H | H | H |
| CH(CH₃)CN | Et | H | H | Me | Me | H | H |
| CH(CH₃)CN | Et | H | H | H | H | Me | Me |
| CH(CH₃)CN | i-Pr | Me | Me | H | H | H | H |
| CH(CH₃)CN | i-Pr | H | H | Me | Me | H | H |
| CH(CH₃)CN | i-Pr | H | H | H | H | Me | Me |
| CH(CH₃)CN | Pr | Me | Me | H | H | H | H |
| CH(CH₃)CN | Pr | H | H | Me | Me | H | H |
| CH(CH₃)CN | Pr | H | H | H | H | Me | Me |
| CH(CH₃)CN | Bu | Me | Me | H | H | H | H |
| CH(CH₃)CN | Bu | H | H | Me | Me | H | H |
| CH(CH₃)CN | Bu | H | H | H | H | Me | Me |
| CH₂CH₂CN | H | Me | Me | H | H | H | H |
| CH₂CH₂CN | H | H | H | Me | Me | H | H |
| CH₂CH₂CN | H | H | H | H | H | Me | Me |
| CH₂CH₂CN | Me | Me | Me | H | H | H | H |
| CH₂CH₂CN | Me | H | H | Me | Me | H | H |
| CH₂CH₂CN | Me | H | H | H | H | Me | Me |
| CH₂CH₂CN | Et | Me | Me | H | H | H | H |
| CH₂CH₂CN | Et | H | H | Me | Me | H | H |
| CH₂CH₂CN | Et | H | H | H | H | Me | Me |
| CH₂CH₂CN | i-Pr | Me | Me | H | H | H | H |
| CH₂CH₂CN | i-Pr | H | H | Me | Me | H | H |
| CH₂CH₂CN | i-Pr | H | H | H | H | Me | Me |
| CH₂CH₂CN | Pr | Me | Me | H | H | H | H |
| CH₂CH₂CN | Pr | H | H | Me | Me | H | H |
| CH₂CH₂CN | Pr | H | H | H | H | Me | Me |
| CH₂CH₂CN | Bu | Me | Me | H | H | H | H |
| CH₂CH₂CN | Bu | H | H | Me | Me | H | H |
| CH₂CH₂CN | Bu | H | H | H | H | Me | Me |

TABLE L61

| R¹ | R² | R³ | R⁴ | R⁵ | R⁶ | R⁷ | R⁸ |
|---|---|---|---|---|---|---|---|
| CH₂CH(CH₃)CN | H | Me | Me | H | H | H | H |
| CH₂CH(CH₃)CN | H | H | H | Me | Me | H | H |
| CH₂CH(CH₃)CN | H | H | H | H | H | Me | Me |
| CH₂CH(CH₃)CN | Me | Me | Me | H | H | H | H |
| CH₂CH(CH₃)CN | Me | H | H | Me | Me | H | H |
| CH₂CH(CH₃)CN | Me | H | H | H | H | Me | Me |
| CH₂CH(CH₃)CN | Et | Me | Me | H | H | H | H |
| CH₂CH(CH₃)CN | Et | H | H | Me | Me | H | H |
| CH₂CH(CH₃)CN | Et | H | H | H | H | Me | Me |
| CH₂CH(CH₃)CN | i-Pr | Me | Me | H | H | H | H |
| CH₂CH(CH₃)CN | i-Pr | H | H | Me | Me | H | H |
| CH₂CH(CH₃)CN | i-Pr | H | H | H | H | Me | Me |
| CH₂CH(CH₃)CN | Pr | Me | Me | H | H | H | H |
| CH₂CH(CH₃)CN | Pr | H | H | Me | Me | H | H |
| CH₂CH(CH₃)CN | Pr | H | H | H | H | Me | M |
| CH₂CH(CH₃)CN | Bu | Me | Me | H | H | H | H |
| CH₂CH(CH₃)CN | Bu | H | H | Me | Me | H | H |
| CH₂CH(CH₃)CN | Bu | H | H | H | H | Me | Me |
| CH₂C(CH₃)₂CN | H | Me | Me | H | H | H | H |
| CH₂C(CH₃)₂CN | H | H | H | Me | Me | H | H |
| CH₂C(CH₃)₂CN | H | H | H | H | H | Me | Me |
| CH₂C(CH₃)₂CN | Me | Me | Me | H | H | H | H |
| CH₂C(CH₃)₂CN | Me | H | H | Me | Me | H | H |
| CH₂C(CH₃)₂CN | Me | H | H | H | H | Me | Me |
| CH₂C(CH₃)₂CN | Et | Me | Me | H | H | H | H |
| CH₂C(CH₃)₂CN | Et | H | H | Me | Me | H | H |
| CH₂C(CH₃)₂CN | Et | H | H | H | H | Me | Me |
| CH₂C(CH₃)₂CN | i-Pr | Me | Me | H | H | H | H |
| CH₂C(CH₃)₂CN | i-Pr | H | H | Me | Me | H | H |
| CH₂C(CH₃)₂CN | i-Pr | H | H | H | H | Me | Me |
| CH₂C(CH₃)₂CN | Pr | Me | Me | H | H | H | H |
| CH₂C(CH₃)₂CN | Pr | H | H | Me | Me | H | H |
| CH₂C(CH₃)₂CN | Pr | H | H | H | H | Me | Me |
| CH₂C(CH₃)₂CN | Bu | Me | Me | H | H | H | H |
| CH₂C(CH₃)₂CN | Bu | H | H | Me | Me | H | H |
| CH₂C(CH₃)₂CN | Bu | H | H | H | H | Me | Me |

TABLE L62

| R¹ | R² | R³ | R⁴ | R⁵ | R⁶ | R⁷ | R⁸ |
|---|---|---|---|---|---|---|---|
| CH₂C(O)NH₂ | H | Me | Me | H | H | H | H |
| CH₂C(O)NH₂ | H | H | H | Me | Me | H | H |
| CH₂C(O)NH₂ | H | H | H | H | H | Me | Me |
| CH₂C(O)NH₂ | Me | Me | Me | H | H | H | H |
| CH₂C(O)NH₂ | Me | H | H | Me | Me | H | H |
| CH₂C(O)NH₂ | Me | H | H | H | H | Me | Me |
| CH₂C(O)NH₂ | Et | Me | Me | H | H | H | H |
| CH₂C(O)NH₂ | Et | H | H | Me | Me | H | H |
| CH₂C(O)NH₂ | Et | H | H | H | H | Me | Me |
| CH₂C(O)NH₂ | i-Pr | Me | Me | H | H | H | H |
| CH₂C(O)NH₂ | i-Pr | H | H | Me | Me | H | H |
| CH₂C(O)NH₂ | i-Pr | H | H | H | H | Me | Me |
| CH₂C(O)NH₂ | Pr | Me | Me | H | H | H | H |
| CH₂C(O)NH₂ | Pr | H | H | Me | Me | H | H |
| CH₂C(O)NH₂ | Pr | H | H | H | H | Me | Me |
| CH₂C(O)NH₂ | Bu | Me | Me | H | H | H | H |
| CH₂C(O)NH₂ | Bu | H | H | Me | Me | H | H |
| CH₂C(O)NH₂ | Bu | H | H | H | H | Me | Me |
| CH₂C(O)NHCH₃ | H | Me | Me | H | H | H | H |
| CH₂C(O)NHCH₃ | H | H | H | Me | Me | H | H |
| CH₂C(O)NHCH₃ | H | H | H | H | H | Me | Me |
| CH₂C(O)NHCH₃ | Me | Me | Me | H | H | H | H |
| CH₂C(O)NHCH₃ | Me | H | H | Me | Me | H | H |
| CH₂C(O)NHCH₃ | Me | H | H | H | H | Me | Me |
| CH₂C(O)NHCH₃ | Et | Me | Me | H | H | H | H |
| CH₂C(O)NHCH₃ | Et | H | H | Me | Me | H | H |
| CH₂C(O)NHCH₃ | Et | H | H | H | H | Me | Me |
| CH₂C(O)NHCH₃ | i-Pr | Me | Me | H | H | H | H |
| CH₂C(O)NHCH₃ | i-Pr | H | H | Me | Me | H | H |
| CH₂C(O)NHCH₃ | i-Pr | H | H | H | H | Me | Me |
| CH₂C(O)NHCH₃ | Pr | Me | Me | H | H | H | H |
| CH₂C(O)NHCH₃ | Pr | H | H | Me | Me | H | H |
| CH₂C(O)NHCH₃ | Pr | H | H | H | H | Me | Me |
| CH₂C(O)NHCH₃ | Bu | Me | Me | H | H | H | H |
| CH₂C(O)NHCH₃ | Bu | H | H | Me | Me | H | H |
| CH₂C(O)NHCH₃ | Bu | H | H | H | H | Me | Me |

TABLE L63

| R¹ | R² | R³ | R⁴ | R⁵ | R⁶ | R⁷ | R⁸ |
|---|---|---|---|---|---|---|---|
| CH₂C(O)N(CH₃)₂ | H | Me | Me | H | H | H | H |
| CH₂C(O)N(CH₃)₂ | H | H | H | Me | Me | H | H |
| CH₂C(O)N(CH₃)₂ | H | H | H | H | H | Me | Me |
| CH₂C(O)N(CH₃)₂ | Me | Me | Me | H | H | H | H |
| CH₂C(O)N(CH₃)₂ | Me | H | H | Me | Me | H | H |
| CH₂C(O)N(CH₃)₂ | Me | H | H | H | H | Me | Me |
| CH₂C(O)N(CH₃)₂ | Et | Me | Me | H | H | H | H |
| CH₂C(O)N(CH₃)₂ | Et | H | H | Me | Me | H | H |
| CH₂C(O)N(CH₃)₂ | Et | H | H | H | H | Me | Me |
| CH₂C(O)N(CH₃)₂ | i-Pr | Me | Me | H | H | H | H |
| CH₂C(O)N(CH₃)₂ | i-Pr | H | H | Me | Me | H | H |
| CH₂C(O)N(CH₃)₂ | i-Pr | H | H | H | H | Me | Me |
| CH₂C(O)N(CH₃)₂ | Pr | Me | Me | H | H | H | H |
| CH₂C(O)N(CH₃)₂ | Pr | H | H | Me | Me | H | H |
| CH₂C(O)N(CH₃)₂ | Pr | H | H | H | H | Me | Me |
| CH₂C(O)N(CH₃)₂ | Bu | Me | Me | H | H | H | H |
| CH₂C(O)N(CH₃)₂ | Bu | H | H | Me | Me | H | H |
| CH₂C(O)N(CH₃)₂ | Bu | H | H | H | H | Me | Me |
| OCH₃ | H | Me | Me | H | H | H | H |
| OCH₃ | H | H | H | Me | Me | H | H |
| OCH₃ | H | H | H | H | H | Me | Me |
| OCH₃ | Me | Me | Me | H | H | H | H |
| OCH₃ | Me | H | H | Me | Me | H | H |
| OCH₃ | Me | H | H | H | H | Me | Me |
| OCH₃ | Et | Me | Me | H | H | H | H |
| OCH₃ | Et | H | H | Me | Me | H | H |
| OCH₃ | Et | H | H | H | H | Me | Me |
| OCH₃ | i-Pr | Me | Me | H | H | H | H |
| OCH₃ | i-Pr | H | H | Me | Me | H | H |
| OCH₃ | i-Pr | H | H | H | H | Me | Me |
| OCH₃ | Pr | Me | Me | H | H | H | H |
| OCH₃ | Pr | H | H | Me | Me | H | H |
| OCH₃ | Pr | H | H | H | H | Me | Me |
| OCH₃ | Bu | Me | Me | H | H | H | H |
| OCH₃ | Bu | H | H | Me | Me | H | H |
| OCH₃ | Bu | H | H | H | H | Me | Me |

TABLE L64

| R¹ | R² | R³ | R⁴ | R⁵ | R⁶ | R⁷ | R⁸ |
|---|---|---|---|---|---|---|---|
| OCH₂CH₃ | H | Me | Me | H | H | H | H |
| OCH₂CH₃ | H | H | H | Me | Me | H | H |
| OCH₂CH₃ | H | H | H | H | H | Me | Me |
| OCH₂CH₃ | Me | Me | Me | H | H | H | H |
| OCH₂CH₃ | Me | H | H | Me | Me | H | H |
| OCH₂CH₃ | Me | H | H | H | H | Me | Me |
| OCH₂CH₃ | Et | Me | Me | H | H | H | H |
| OCH₂CH₃ | Et | H | H | Me | Me | H | H |
| OCH₂CH₃ | Et | H | H | H | H | Me | Me |
| OCH₂CH₃ | i-Pr | Me | Me | H | H | H | H |
| OCH₂CH₃ | i-Pr | H | H | Me | Me | H | H |
| OCH₂CH₃ | i-Pr | H | H | H | H | Me | Me |
| OCH₂CH₃ | Pr | Me | Me | H | H | H | H |
| OCH₂CH₃ | Pr | H | H | Me | Me | H | H |
| OCH₂CH₃ | Pr | H | H | H | H | Me | Me |
| OCH₂CH₃ | Bu | Me | Me | H | H | H | H |
| OCH₂CH₃ | Bu | H | H | Me | Me | H | H |
| OCH₂CH₃ | Bu | H | H | H | H | Me | Me |
| OCH₂CH₂CH₃ | H | Me | Me | H | H | H | H |
| OCH₂CH₂CH₃ | H | H | H | Me | Me | H | H |
| OCH₂CH₂CH₃ | H | H | H | H | H | Me | Me |
| OCH₂CH₂CH₃ | Me | Me | Me | H | H | H | H |
| OCH₂CH₂CH₃ | Me | H | H | Me | Me | H | H |
| OCH₂CH₂CH₃ | Me | H | H | H | H | Me | Me |
| OCH₂CH₂CH₃ | Et | Me | Me | H | H | H | H |
| OCH₂CH₂CH₃ | Et | H | H | Me | Me | H | H |
| OCH₂CH₂CH₃ | Et | H | H | H | H | Me | Me |
| OCH₂CH₂CH₃ | i-Pr | Me | Me | H | H | H | H |
| OCH₂CH₂CH₃ | i-Pr | H | H | Me | Me | H | H |
| OCH₂CH₂CH₃ | i-Pr | H | H | H | H | Me | Me |
| OCH₂CH₂CH₃ | Pr | Me | Me | H | H | H | H |
| OCH₂CH₂CH₃ | Pr | H | H | Me | Me | H | H |
| OCH₂CH₂CH₃ | Pr | H | H | H | H | Me | Me |
| OCH₂CH₂CH₃ | Bu | Me | Me | H | H | H | H |
| OCH₂CH₂CH₃ | Bu | H | H | Me | Me | H | H |
| OCH₂CH₂CH₃ | Bu | H | H | H | H | Me | Me |

TABLE L65

| R¹ | R² | R³ | R⁴ | R⁵ | R⁶ | R⁷ | R⁸ |
|---|---|---|---|---|---|---|---|
| OCH₂CF₃ | H | Me | Me | H | H | H | H |
| OCH₂CF₃ | H | H | H | Me | Me | H | H |
| OCH₂CF₃ | H | H | H | H | H | Me | Me |
| OCH₂CF₃ | Me | Me | Me | H | H | H | H |
| OCH₂CF₃ | Me | H | H | Me | Me | H | H |
| OCH₂CF₃ | Me | H | H | H | H | Me | Me |
| OCH₂CF₃ | Et | Me | Me | H | H | H | H |
| OCH₂CF₃ | Et | H | H | Me | Me | H | H |
| OCH₂CF₃ | Et | H | H | H | H | Me | Me |
| OCH₂CF₃ | i-Pr | Me | Me | H | H | H | H |
| OCH₂CF₃ | i-Pr | H | H | Me | Me | H | H |
| OCH₂CF₃ | i-Pr | H | H | H | H | Me | Me |
| OCH₂CF₃ | Pr | Me | Me | H | H | H | H |
| OCH₂CF₃ | Pr | H | H | Me | Me | H | H |
| OCH₂CF₃ | Pr | H | H | H | H | Me | M |
| OCH₂CF₃ | Bu | Me | Me | H | H | H | H |
| OCH₂CF₃ | Bu | H | H | Me | Me | H | H |
| OCH₂CF₃ | Bu | H | H | H | H | Me | Me |
| OCH₂CH₂CF₃ | H | Me | Me | H | H | H | H |
| OCH₂CH₂CF₃ | H | H | H | Me | Me | H | H |
| OCH₂CH₂CF₃ | H | H | H | H | H | Me | Me |
| OCH₂CH₂CF₃ | Me | Me | Me | H | H | H | H |
| OCH₂CH₂CF₃ | Me | H | H | Me | Me | H | H |
| OCH₂CH₂CF₃ | Me | H | H | H | H | Me | Me |
| OCH₂CH₂CF₃ | Et | Me | Me | H | H | H | H |
| OCH₂CH₂CF₃ | Et | H | H | Me | Me | H | H |
| OCH₂CH₂CF₃ | Et | H | H | H | H | Me | Me |
| OCH₂CH₂CF₃ | i-Pr | Me | Me | H | H | H | H |
| OCH₂CH₂CF₃ | i-Pr | H | H | Me | Me | H | H |
| OCH₂CH₂CF₃ | i-Pr | H | H | H | H | Me | Me |
| OCH₂CH₂CF₃ | Pr | Me | Me | H | H | H | H |
| OCH₂CH₂CF₃ | Pr | H | H | Me | Me | H | H |
| OCH₂CH₂CF₃ | Pr | H | H | H | H | Me | Me |
| OCH₂CH₂CF₃ | Bu | Me | Me | H | H | H | H |
| OCH₂CH₂CF₃ | Bu | H | H | Me | Me | H | H |
| OCH₂CH₂CF₃ | Bu | H | H | H | H | Me | Me |

TABLE L66

| R¹ | R² | R³ | R⁴ | R⁵ | R⁶ | R⁷ | R⁸ |
|---|---|---|---|---|---|---|---|
| C(O)CH₃ | H | Me | Me | H | H | H | H |
| C(O)CH₃ | H | H | H | Me | Me | H | H |
| C(O)CH₃ | H | H | H | H | H | Me | Me |
| C(O)CH₃ | Me | Me | Me | H | H | H | H |
| C(O)CH₃ | Me | H | H | Me | Me | H | H |
| C(O)CH₃ | Me | H | H | H | H | Me | Me |
| C(O)CH₃ | Et | Me | Me | H | B | H | H |
| C(O)CH₃ | Et | H | H | Me | Me | H | H |
| C(O)CH₃ | Et | H | H | H | H | Me | Me |
| C(O)CH₃ | i-Pr | Me | Me | H | H | H | H |
| C(O)CH₃ | i-Pr | H | H | Me | Me | H | H |
| C(O)CH₃ | i-Pr | H | H | H | H | Me | Me |
| C(O)CH₃ | Pr | Me | Me | H | H | H | H |
| C(O)CH₃ | Pr | H | H | Me | Me | H | H |
| C(O)CH₃ | Pr | H | H | H | H | Me | Me |
| C(O)CH₃ | Bu | Me | Me | H | H | H | H |
| C(O)CH₃ | Bu | H | H | Me | Me | H | H |
| C(O)CH₃ | Bu | H | H | H | H | Me | Me |
| C(O)CH₂CH₃ | H | Me | Me | H | H | H | H |
| C(O)CH₂CH₃ | H | H | H | Me | Me | H | H |
| C(O)CH₂CH₃ | H | H | H | H | H | Me | Me |
| C(O)CH₂CH₃ | Me | Me | Me | H | H | H | H |
| C(O)CH₂CH₃ | Me | H | H | Me | Me | H | H |
| C(O)CH₂CH₃ | Me | H | H | H | H | Me | Me |
| C(O)CH₂CH₃ | Et | Me | Me | H | H | H | H |
| C(O)CH₂CH₃ | Et | H | H | Me | Me | H | H |
| C(O)CH₂CH₃ | Et | H | H | H | H | Me | Me |
| C(O)CH₂CH₃ | i-Pr | Me | Me | H | H | H | H |
| C(O)CH₂CH₃ | i-Pr | H | H | Me | Me | H | H |
| C(O)CH₂CH₃ | i-Pr | H | H | H | H | Me | Me |
| C(O)CH₂CH₃ | Pr | Me | Me | H | H | H | H |
| C(O)CH₂CH₃ | Pr | H | H | Me | Me | H | H |
| C(O)CH₂CH₃ | Pr | H | H | H | H | Me | Me |
| C(O)CH₂CH₃ | Bu | Me | Me | H | H | H | H |
| C(O)CH₂CH₃ | Bu | H | H | Me | Me | H | H |
| C(O)CH₂CH₃ | Bu | H | H | H | H | Me | Me |

TABLE L67

| R¹ | R² | R³ | R⁴ | R⁵ | R⁶ | R⁷ | R⁸ |
|---|---|---|---|---|---|---|---|
| C(O)CF₃ | H | Me | Me | H | H | H | H |
| C(O)CF₃ | H | H | H | Me | Me | H | H |
| C(O)CF₃ | H | H | H | H | H | Me | Me |
| C(O)CF₃ | Me | Me | Me | H | H | H | H |
| C(O)CF₃ | Me | H | H | Me | Me | H | H |
| C(O)CF₃ | Me | H | H | H | H | Me | Me |
| C(O)CF₃ | Et | Me | Me | H | H | H | H |
| C(O)CF₃ | Et | H | H | Me | Me | H | H |
| C(O)CF₃ | Et | H | H | H | H | Me | Me |
| C(O)CF₃ | i-Pr | Me | Me | H | H | H | H |
| C(O)CF₃ | i-Pr | H | H | Me | Me | H | H |
| C(O)CF₃ | i-Pr | H | H | H | H | Me | Me |
| C(O)CF₃ | Pr | Me | Me | H | H | H | H |
| C(O)CF₃ | Pr | H | H | Me | Me | H | H |
| C(O)CF₃ | Pr | H | H | H | H | Me | Me |
| C(O)CF₃ | Bu | Me | Me | H | H | H | H |
| C(O)CF₃ | Bu | H | H | Me | Me | H | H |
| C(O)CF₃ | Bu | H | H | H | H | Me | Me |
| C(O)CH₂CF₃ | H | Me | Me | H | H | H | H |
| C(O)CH₂CF₃ | H | H | H | Me | Me | H | H |
| C(O)CH₂CF₃ | H | H | H | H | H | Me | Me |
| C(O)CH₂CF₃ | Me | Me | Me | H | H | H | H |
| C(O)CH₂CF₃ | Me | H | H | Me | Me | H | H |
| C(O)CH₂CF₃ | Me | H | H | H | H | Me | Me |
| C(O)CH₂CF₃ | Et | Me | Me | H | H | H | H |
| C(O)CH₂CF₃ | Et | H | H | Me | Me | H | H |
| C(O)CH₂CF₃ | Et | H | H | H | H | Me | Me |
| C(O)CH₂CF₃ | i-Pr | Me | Me | H | H | H | H |
| C(O)CH₂CF₃ | i-Pr | H | H | Me | Me | H | H |
| C(O)CH₂CF₃ | i-Pr | H | H | H | H | Me | Me |
| C(O)CH₂CF₃ | Pr | Me | Me | H | H | H | H |
| C(O)CH₂CF₃ | Pr | H | H | Me | Me | H | H |
| C(O)CH₂CF₃ | Pr | H | H | H | H | Me | Me |
| C(O)CH₂CF₃ | Bu | Me | Me | H | H | H | H |
| C(O)CH₂CF₃ | Bu | H | H | Me | Me | H | H |
| C(O)CH₂CF₃ | Bu | H | H | H | H | Me | Me |

TABLE L68

| R¹ | R² | R³ | R⁴ | R⁵ | R⁶ | R⁷ | R⁸ |
|---|---|---|---|---|---|---|---|
| C(O)OCH₃ | H | Me | Me | H | H | H | H |
| C(O)OCH₃ | H | H | H | Me | Me | H | H |
| C(O)OCH₃ | H | H | H | H | H | Me | Me |
| C(O)OCH₃ | Me | Me | Me | H | H | H | H |
| C(O)OCH₃ | Me | H | H | Me | Me | H | H |
| C(O)OCH₃ | Me | H | H | H | H | Me | Me |
| C(O)OCH₃ | Et | Me | Me | H | H | H | H |
| C(O)OCH₃ | Et | H | H | Me | Me | H | H |
| C(O)OCH₃ | Et | H | H | H | H | Me | Me |
| C(O)OCH₃ | i-Pr | Me | Me | H | H | H | H |
| C(O)OCH₃ | i-Pr | H | H | Me | Me | H | H |
| C(O)OCH₃ | i-Pr | H | H | H | H | Me | Me |
| C(O)OCH₃ | Pr | Me | Me | H | H | H | H |
| C(O)OCH₃ | Pr | H | H | Me | Me | H | H |
| C(O)OCH₃ | Pr | H | H | H | H | Me | Me |
| C(O)OCH₃ | Bu | Me | Me | H | H | H | H |
| C(O)OCH₃ | Bu | H | H | Me | Me | H | H |
| C(O)OCH₃ | Bu | H | H | H | H | Me | Me |
| C(O)OCH₂CH₃ | H | Me | Me | H | H | H | H |
| C(O)OCH₂CH₃ | H | H | H | Me | Me | H | H |
| C(O)OCH₂CH₃ | H | H | H | H | H | Me | Me |
| C(O)OCH₂CH₃ | Me | Me | Me | H | H | H | H |
| C(O)OCH₂CH₃ | Me | H | H | Me | Me | H | H |
| C(O)OCH₂CH₃ | Me | H | H | H | H | Me | Me |
| C(O)OCH₂CH₃ | Et | Me | Me | H | H | H | H |
| C(O)OCH₂CH₃ | Et | H | H | Me | Me | H | H |
| C(O)OCH₂CH₃ | Et | H | H | H | H | Me | Me |
| C(O)OCH₂CH₃ | i-Pr | Me | Me | H | H | H | H |
| C(O)OCH₂CH₃ | i-Pr | H | H | Me | Me | H | H |
| C(O)OCH₂CH₃ | i-Pr | H | H | H | H | Me | Me |
| C(O)OCH₂CH₃ | Pr | Me | Me | H | H | H | H |
| C(O)OCH₂CH₃ | Pr | H | H | Me | Me | H | H |
| C(O)OCH₂CH₃ | Pr | H | H | H | H | Me | Me |
| C(O)OCH₂CH₃ | Bu | Me | Me | H | H | H | H |
| C(O)OCH₂CH₃ | Bu | H | H | Me | Me | H | H |
| C(O)OCH₂CH₃ | Bu | H | H | H | H | Me | Me |

TABLE L69

| R¹ | R² | R³ | R⁴ | R⁵ | R⁶ | R⁷ | R⁸ |
|---|---|---|---|---|---|---|---|
| C(O)OCF₃ | H | Me | Me | H | H | H | H |
| C(O)OCF₃ | H | H | H | Me | Me | H | H |
| C(O)OCF₃ | H | H | H | H | H | Me | Me |
| C(O)OCF₃ | Me | Me | Me | H | H | H | H |
| C(O)OCF₃ | Me | H | H | Me | Me | H | H |
| C(O)OCF₃ | Me | H | H | H | H | Me | Me |
| C(O)OCF₃ | Et | Me | Me | H | H | H | H |
| C(O)OCF₃ | Et | H | H | Me | Me | H | H |
| C(O)OCF₃ | Et | H | H | H | H | Me | Me |
| C(O)OCF₃ | i-Pr | Me | Me | H | H | H | H |
| C(O)OCF₃ | i-Pr | H | H | Me | Me | H | H |
| C(O)OCF₃ | i-Pr | H | H | H | H | Me | Me |
| C(O)OCF₃ | Pr | Me | Me | H | H | H | H |
| C(O)OCF₃ | Pr | H | H | Me | Me | H | H |
| C(O)OCF₃ | Pr | H | H | H | H | Me | Me |
| C(O)OCF₃ | Bu | Me | Me | H | H | H | H |
| C(O)OCF₃ | Bu | H | H | Me | Me | H | H |
| C(O)OCF₃ | Bu | H | H | H | H | Me | Me |
| C(O)OCH₂CF₃ | H | Me | Me | H | H | H | H |
| C(O)OCH₂CF₃ | H | H | H | Me | Me | H | H |
| C(O)OCH₂CF₃ | H | H | H | H | H | Me | Me |
| C(O)OCH₂CF₃ | Me | Me | Me | H | H | H | H |
| C(O)OCH₂CF₃ | Me | H | H | Me | Me | H | H |
| C(O)OCH₂CF₃ | Me | H | H | H | H | Me | Me |
| C(O)OCH₂CF₃ | Et | Me | Me | H | H | H | H |
| C(O)OCH₂CF₃ | Et | H | H | Me | Me | H | H |
| C(O)OCH₂CF₃ | Et | H | H | H | H | Me | Me |
| C(O)OCH₂CF₃ | i-Pr | Me | Me | H | H | H | H |
| C(O)OCH₂CF₃ | i-Pr | H | H | Me | Me | H | H |
| C(O)OCH₂CF₃ | i-Pr | H | H | H | H | Me | Me |
| C(O)OCH₂CF₃ | Pr | Me | Me | H | H | H | H |
| C(O)OCH₂CF₃ | Pr | H | H | Me | Me | H | H |
| C(O)OCH₂CF₃ | Pr | H | H | H | H | Me | Me |
| C(O)OCH₂CF₃ | Bu | Me | Me | H | H | H | H |
| C(O)OCH₂CF₃ | Bu | H | H | Me | Me | H | H |
| C(O)OCH₂CF₃ | Bu | H | H | H | H | Me | Me |

TABLE L70

| R¹ | R² | R³ | R⁴ | R⁵ | R⁶ | R⁷ | R⁸ |
|---|---|---|---|---|---|---|---|
| S(O)₂CH₃ | H | Me | Me | H | H | H | H |
| S(O)₂CH₃ | H | H | H | Me | Me | H | H |
| S(O)₂CH₃ | H | H | H | H | H | Me | Me |
| S(O)₂CH₃ | Me | Me | Me | H | H | H | H |
| S(O)₂CH₃ | Me | H | H | Me | Me | H | H |
| S(O)₂CH₃ | Me | H | H | H | H | Me | Me |
| S(O)₂CH₃ | Et | Me | Me | H | H | H | H |
| S(O)₂CH₃ | Et | H | H | Me | Me | H | H |
| S(O)₂CH₃ | Et | H | H | H | H | Me | Me |
| S(O)₂CH₃ | i-Pr | Me | Me | H | H | H | H |
| S(O)₂CH₃ | i-Pr | H | H | Me | Me | H | H |
| S(O)₂CH₃ | i-Pr | H | H | H | H | Me | Me |
| S(O)₂CH₃ | Pr | Me | Me | H | H | H | H |
| S(O)₂CH₃ | Pr | H | H | Me | Me | H | H |
| S(O)₂CH₃ | Pr | H | H | H | H | Me | Me |
| S(O)₂CH₃ | Bu | Me | Me | H | H | H | H |
| S(O)₂CH₃ | Bu | H | H | Me | Me | H | H |
| S(O)₂CH₃ | Bu | H | H | H | H | Me | Me |
| S(O)₂CH₂CH₃ | H | Me | Me | H | H | H | H |
| S(O)₂CH₂CH₃ | H | H | H | Me | Me | H | H |
| S(O)₂CH₂CH₃ | H | H | H | H | H | Me | Me |
| S(O)₂CH₂CH₃ | Me | Me | Me | H | H | H | H |
| S(O)₂CH₂CH₃ | Me | H | H | Me | Me | H | H |
| S(O)₂CH₂CH₃ | Me | H | H | H | H | Me | Me |
| S(O)₂CH₂CH₃ | Et | Me | Me | H | H | H | H |
| S(O)₂CH₂CH₃ | Et | H | H | Me | Me | H | H |
| S(O)₂CH₂CH₃ | Et | H | H | H | H | Me | Me |
| S(O)₂CH₂CH₃ | i-Pr | Me | Me | H | H | H | H |
| S(O)₂CH₂CH₃ | i-Pr | H | H | Me | Me | H | H |
| S(O)₂CH₂CH₃ | i-Pr | H | H | H | H | Me | Me |
| S(O)₂CH₂CH₃ | Pr | Me | Me | H | H | H | H |
| S(O)₂CH₂CH₃ | Pr | H | H | Me | Me | H | H |
| S(O)₂CH₂CH₃ | Pr | H | H | H | H | Me | Me |
| S(O)₂CH₂CH₃ | Bu | Me | Me | H | H | H | H |
| S(O)₂CH₂CH₃ | Bu | H | H | Me | Me | H | H |
| S(O)₂CH₂CH₃ | Bu | H | H | H | H | Me | Me |

TABLE L71

| R¹ | R² | R³ | R⁴ | R⁵ | R⁶ | R⁷ | R⁸ |
|---|---|---|---|---|---|---|---|
| S(O)₂CF₃ | H | Me | Me | H | H | H | H |
| S(O)₂CF₃ | H | H | H | Me | Me | H | H |
| S(O)₂CF₃ | H | H | H | H | H | Me | Me |
| S(O)₂CF₃ | Me | Me | Me | H | H | H | H |
| S(O)₂CF₃ | Me | H | H | Me | Me | H | H |
| S(O)₂CF₃ | Me | H | H | H | H | Me | Me |
| S(O)₂CF₃ | Et | Me | Me | H | H | H | H |
| S(O)₂CF₃ | Et | H | H | Me | Me | H | H |
| S(O)₂CF₃ | Et | H | H | H | H | Me | Me |
| S(O)₂CF₃ | i-Pr | Me | Me | H | H | H | H |
| S(O)₂CF₃ | i-Pr | H | H | Me | Me | H | H |
| S(O)₂CF₃ | i-Pr | H | H | H | H | Me | Me |
| S(O)₂CF₃ | Pr | Me | Me | H | H | H | H |
| S(O)₂CF₃ | Pr | H | H | Me | Me | H | H |
| S(O)₂CF₃ | Pr | H | H | H | H | Me | Me |
| S(O)₂CF₃ | Bu | Me | Me | H | H | H | H |
| S(O)₂CF₃ | Bu | H | H | Me | Me | H | H |
| S(O)₂CF₃ | Bu | H | H | H | H | Me | Me |
| S(O)₂CH₂CF₃ | H | Me | Me | H | H | H | H |
| S(O)₂CH₂CF₃ | H | H | H | Me | Me | H | H |
| S(O)₂CH₂CF₃ | H | H | H | H | H | Me | Me |
| S(O)₂CH₂CF₃ | Me | Me | Me | H | H | H | H |
| S(O)₂CH₂CF₃ | Me | H | H | Me | Me | H | H |
| S(O)₂CH₂CF₃ | Me | H | H | H | H | Me | Me |
| S(O)₂CH₂CF₃ | Et | Me | Me | H | H | H | H |
| S(O)₂CH₂CF₃ | Et | H | H | Me | Me | H | H |
| S(O)₂CH₂CF₃ | Et | H | H | H | H | Me | Me |
| S(O)₂CH₂CF₃ | i-Pr | Me | Me | H | H | H | H |
| S(O)₂CH₂CF₃ | i-Pr | H | H | Me | Me | H | H |
| S(O)₂CH₂CF₃ | i-Pr | H | H | H | H | Me | Me |
| S(O)₂CH₂CF₃ | Pr | Me | Me | H | H | H | H |
| S(O)₂CH₂CF₃ | Pr | H | H | Me | Me | H | H |
| S(O)₂CH₂CF₃ | Pr | H | H | H | H | Me | Me |
| S(O)₂CH₂CF₃ | Bu | Me | Me | H | H | H | H |
| S(O)₂CH₂CF₃ | Bu | H | H | Me | Me | H | H |
| S(O)₂CH₂CF₃ | Bu | H | H | H | H | Me | Me |

TABLE L72

| | | | | | | | |
|---|---|---|---|---|---|---|---|
| R¹ | R² | R³ | R⁴ | R⁵ | R⁶ | R⁷ | R⁸ |

| | | | | | | |
|---|---|---|---|---|---|---|
| —CH₂CH₂— | Me | Me | H | H | H | H |
| —CH₂CH₂— | H | H | Me | Me | H | H |
| —CH₂CH₂— | H | H | H | H | Me | Me |
| —CH₂CH₂CH₂— | Me | Me | H | H | H | H |
| —CH₂CH₂CH₂— | H | H | Me | Me | H | H |
| —CH₂CH₂CH₂— | H | H | H | H | Me | Me |
| —CH₂CH(CH₃)CH₂— | Me | Me | H | H | H | H |
| —CH₂CH(CH₃)CH₂— | H | H | Me | Me | H | H |
| —CH₂CH(CH₃)CH₂— | H | H | H | H | Me | Me |
| —CH₂C(CH₃)₂CH₂— | Me | Me | H | H | H | H |
| —CH₂C(CH₃)₂CH₂— | H | H | Me | Me | H | H |
| —CH₂C(CH₃)₂CH₂— | H | H | H | H | Me | Me |
| —CH₂CH₂CH₂CH₂— | Me | Me | H | H | H | H |
| —CH₂CH₂CH₂CH₂— | H | H | Me | Me | H | H |
| —CH₂CH₂CH₂CH₂— | H | H | H | H | Me | Me |
| —CH₂CH(CH₃)CH₂CH₂— | Me | Me | H | H | H | H |
| —CH₂CH(CH₃)CH₂CH₂— | H | H | Me | Me | H | H |
| —CH₂CH(CH₃)CH₂CH₂— | H | H | H | H | Me | Me |
| —CH₂C(CH₃)₂CH₂CH₂— | Me | Me | H | H | H | H |
| —CH₂C(CH₃)₂CH₂CH₂— | H | H | Me | Me | H | H |
| —CH₂C(CH₃)₂CH₂CH₂— | H | H | H | H | Me | Me |
| —CH₂CH₂CH₂CH₂CH₂— | Me | Me | H | H | H | H |
| —CH₂CH₂CH₂CH₂CH₂— | H | H | Me | Me | H | H |
| —CH₂CH₂CH₂CH₂CH₂— | H | H | H | H | Me | Me |
| —CH₂CH₂OCH₂CH₂— | Me | Me | H | H | H | H |
| —CH₂CH₂OCH₂CH₂— | H | H | Me | Me | H | H |
| —CH₂CH₂OCH₂CH₂— | H | H | H | H | Me | Me |
| —CH₂CH₂SCH₂CH₂— | Me | Me | H | H | H | H |
| —CH₂CH₂SCH₂CH₂— | H | H | Me | Me | H | H |
| —CH₂CH₂SCH₂CH₂— | H | H | H | H | Me | Me |
| —CH₂CH₂NHCH₂CH₂— | Me | Me | H | H | H | H |
| —CH₂CH₂NHCH₂CH₂— | H | H | Me | Me | H | H |
| —CH₂CH₂NHCH₂CH₂— | H | H | H | H | Me | Me |
| —CH₂CH₂N(CH₃)CH₂CH₂— | Me | Me | H | H | H | H |
| —CH₂CH₂N(CH₃)CH₂CH₂— | H | H | Me | Me | H | H |
| —CH₂CH₂N(CH₃)CH₂CH₂— | H | H | H | H | Me | Me |

TABLE L73

| R¹ | R² | R³ | R⁴ | R⁵ | R⁶ | R⁷ | R⁸ |
|---|---|---|---|---|---|---|---|
| CH₂—Ph | H | Me | Me | H | H | H | H |
| CH₂—Ph | H | H | H | Me | Me | H | H |
| CH₂—Ph | H | H | H | H | H | Me | Me |
| CH₂—Ph | Me | Me | Me | H | H | H | H |
| CH₂—Ph | Me | H | H | Me | Me | H | H |
| CH₂—Ph | Me | H | H | H | H | Me | Me |
| CH₂—Ph | Et | Me | Me | H | H | H | H |
| CH₂—Ph | Et | H | H | Me | Me | H | H |
| CH₂—Ph | Et | H | H | H | H | Me | Me |
| CH₂—Ph | i-Pr | Me | Me | H | H | H | H |
| CH₂—Ph | i-Pr | H | H | Me | Me | H | H |
| CH₂—Ph | i-Pr | H | H | H | H | Me | Me |
| CH₂—Ph | Pr | Me | Me | H | H | H | H |
| CH₂—Ph | Pr | H | H | Me | Me | H | H |
| CH₂—Ph | Pr | H | H | H | H | Me | Me |
| CH₂—Ph | Bu | Me | Me | H | H | H | H |
| CH₂—Ph | Bu | H | H | Me | Me | H | H |
| CH₂—Ph | Bu | H | H | H | H | Me | Me |
| CH₂-2-CH₃—Ph | H | Me | Me | H | H | H | H |
| CH₂-2-CH₃—Ph | H | H | H | Me | Me | H | H |
| CH₂-2-CH₃—Ph | H | H | H | H | H | Me | Me |
| CH₂-2-CH₃—Ph | Me | Me | Me | H | H | H | H |
| CH₂-2-CH₃—Ph | Me | H | H | Me | Me | H | H |
| CH₂-2-CH₃—Ph | Me | H | H | H | H | Me | Me |
| CH₂-2-CH₃—Ph | Et | Me | Me | H | H | H | H |
| CH₂-2-CH₃—Ph | Et | H | H | Me | Me | H | H |
| CH₂-2-CH₃—Ph | Et | H | H | H | H | Me | Me |
| CH₂-2-CH₃—Ph | i-Pr | Me | Me | H | H | H | H |
| CH₂-2-CH₃—Ph | i-Pr | H | H | Me | Me | H | H |
| CH₂-2-CH₃—Ph | i-Pr | H | H | H | H | Me | Me |
| CH₂-2-CH₃—Ph | Pr | Me | Me | H | H | H | H |
| CH₂-2-CH₃—Ph | Pr | H | H | Me | Me | H | H |
| CH₂-2-CH₃—Ph | Pr | H | H | H | H | Me | Me |
| CH₂-2-CH₃—Ph | Bu | Me | Me | H | H | H | H |
| CH₂-2-CH₃—Ph | Bu | H | H | Me | Me | H | H |
| CH₂-2-CH₃—Ph | Bu | H | H | H | H | Me | Me |

TABLE L74

| R¹ | R² | R³ | R⁴ | R⁵ | R⁶ | R⁷ | R⁸ |
|---|---|---|---|---|---|---|---|
| CH₂-3-CH₃—Ph | H | Me | Me | H | H | H | H |
| CH₂-3-CH₃—Ph | H | H | H | Me | Me | H | H |
| CH₂-3-CH₃—Ph | H | H | H | H | H | Me | Me |
| CH₂-3-CH₃—Ph | Me | Me | Me | H | H | H | H |
| CH₂-3-CH₃—Ph | Me | H | H | Me | Me | H | H |
| CH₂-3-CH₃—Ph | Me | H | H | H | H | Me | Me |
| CH₂-3-CH₃—Ph | Et | Me | Me | H | H | H | H |
| CH₂-3-CH₃—Ph | Et | H | H | Me | Me | H | H |
| CH₂-3-CH₃—Ph | Et | H | H | H | H | Me | Me |
| CH₂-3-CH₃—Ph | i-Pr | Me | Me | H | H | H | H |
| CH₂-3-CH₃—Ph | i-Pr | H | H | Me | Me | H | H |
| CH₂-3-CH₃—Ph | i-Pr | H | H | H | H | Me | Me |
| CH₂-3-CH₃—Ph | Pr | Me | Me | H | H | H | H |
| CH₂-3-CH₃—Ph | Pr | H | H | Me | Me | H | H |
| CH₂-3-CH₃—Ph | Pr | H | H | H | H | Me | Me |
| CH₂-3-CH₃—Ph | Bu | Me | Me | H | H | H | H |
| CH₂-3-CH₃—Ph | Bu | H | H | Me | Me | H | H |
| CH₂-3-CH₃—Ph | Bu | H | H | H | H | Me | Me |
| CH₂-4-CH₃—Ph | H | Me | Me | H | H | H | H |
| CH₂-4-CH₃—Ph | H | H | H | Me | Me | H | H |
| CH₂-4-CH₃—Ph | H | H | H | H | H | Me | Me |
| CH₂-4-CH₃—Ph | Me | Me | Me | H | H | H | H |
| CH₂-4-CH₃—Ph | Me | H | H | Me | Me | H | H |
| CH₂-4-CH₃—Ph | Me | H | H | H | H | Me | Me |
| CH₂-4-CH₃—Ph | Et | Me | Me | H | H | H | H |
| CH₂-4-CH₃—Ph | Et | H | H | Me | Me | H | H |
| CH₂-4-CH₃—Ph | Et | H | H | H | H | Me | Me |
| CH₂-4-CH₃—Ph | i-Pr | Me | Me | H | H | H | H |
| CH₂-4-CH₃—Ph | i-Pr | H | H | Me | Me | H | H |
| CH₂-4-CH₃—Ph | i-Pr | H | H | H | H | Me | Me |
| CH₂-4-CH₃—Ph | Pr | Me | Me | H | H | H | H |
| CH₂-4-CH₃—Ph | Pr | H | H | Me | Me | H | H |
| CH₂-4-CH₃—Ph | Pr | H | H | H | H | Me | Me |
| CH₂-4-CH₃—Ph | Bu | Me | Me | H | H | H | H |
| CH₂-4-CH₃—Ph | Bu | H | H | Me | Me | H | H |
| CH₂-4-CH₃—Ph | Bu | H | H | H | H | Me | Me |

TABLE L75

| R¹ | R² | R³ | R⁴ | R⁵ | R⁶ | R⁷ | R⁸ |
|---|---|---|---|---|---|---|---|
| CH₂-2-OCH₃—Ph | H | Me | Me | H | H | H | H |
| CH₂-2-OCH₃—Ph | H | H | H | Me | Me | H | H |
| CH₂-2-OCH₃—Ph | H | H | H | H | H | Me | Me |
| CH₂-2-OCH₃—Ph | Me | Me | Me | H | H | H | H |
| CH₂-2-OCH₃—Ph | Me | H | H | Me | Me | H | H |
| CH₂-2-OCH₃—Ph | Me | H | H | H | H | Me | Me |
| CH₂-2-OCH₃—Ph | Et | Me | Me | H | H | H | H |
| CH₂-2-OCH₃—Ph | Et | H | H | Me | M? | H | H |
| CH₂-2-OCH₃—Ph | Et | H | H | H | H | Me | Me |
| CH₂-2-OCH₃—Ph | i-Pr | Me | Me | H | H | H | H |
| CH₂-2-OCH₃—Ph | i-Pr | H | H | Me | Me | H | H |
| CH₂-2-OCH₃—Ph | i-Pr | H | H | H | H | Me | Me |
| CH₂-2-OCH₃—Ph | Pr | Me | Me | H | H | H | H |
| CH₂-2-OCH₃—Ph | Pr | H | H | Me | Me | H | H |
| CH₂-2-OCH₃—Ph | Pr | H | H | H | H | Me | Me |
| CH₂-2-OCH₃—Ph | Bu | Me | Me | H | H | H | H |
| CH₂-2-OCH₃—Ph | Bu | H | H | Me | Me | H | H |
| CH₂-2-OCH₃—Ph | Bu | H | H | H | H | Me | Me |
| CH₂-3-OCH₃—Ph | H | Me | Me | H | H | H | H |
| CH₂-3-OCH₃—Ph | H | H | H | Me | Me | H | H |
| CH₂-3-OCH₃—Ph | H | H | H | H | H | Me | Me |
| CH₂-3-OCH₃—Ph | Me | Me | Me | H | H | H | H |
| CH₂-3-OCH₃—Ph | Me | H | H | Me | Me | H | H |
| CH₂-3-OCH₃—Ph | Me | H | H | H | H | Me | Me |
| CH₂-3-OCH₃—Ph | Et | Me | Me | H | H | H | H |
| CH₂-3-OCH₃—Ph | Et | H | H | Me | Me | H | H |
| CH₂-3-OCH₃—Ph | Et | H | H | H | H | Me | Me |
| CH₂-3-OCH₃—Ph | i-Pr | Me | Me | H | H | H | H |
| CH₂-3-OCH₃—Ph | i-Pr | H | H | Me | Me | H | H |
| CH₂-3-OCH₃—Ph | i-Pr | H | H | H | H | Me | Me |
| CH₂-3-OCH₃—Ph | Pr | Me | Me | H | H | H | H |
| CH₂-3-OCH₃—Ph | Pr | H | H | Me | Me | H | H |
| CH₂-3-OCH₃—Ph | Pr | H | H | H | H | Me | Me |
| CH₂-3-OCH₃—Ph | Bu | Me | Me | H | H | H | H |
| CH₂-3-OCH₃—Ph | Bu | H | H | Me | Me | H | H |
| CH₂-3-OCH₃—Ph | Bu | H | H | H | H | Me | Me |

TABLE L76

| R¹ | R² | R³ | R⁴ | R⁵ | R⁶ | R⁷ | R⁸ |
|---|---|---|---|---|---|---|---|
| CH₂-4-OCH₃—Ph | H | Me | Me | H | H | H | H |
| CH₂-4-OCH₃—Ph | H | H | H | Me | Me | H | H |
| CH₂-4-OCH₃—Ph | H | H | H | H | H | Me | Me |
| CH₂-4-OCH₃—Ph | Me | Me | Me | H | H | H | H |
| CH₂-4-OCH₃—Ph | Me | H | H | Me | Me | H | H |
| CH₂-4-OCH₃—Ph | Me | H | H | H | H | Me | Me |
| CH₂-4-OCH₃—Ph | Et | Me | Me | H | H | H | H |
| CH₂-4-OCH₃—Ph | Et | H | H | Me | Me | H | H |
| CH₂-4-OCH₃—Ph | Et | H | H | H | H | Me | Me |
| CH₂-4-OCH₃—Ph | i-Pr | Me | Me | H | H | H | H |
| CH₂-4-OCH₃—Ph | i-Pr | H | H | Me | Me | H | H |
| CH₂-4-OCH₃—Ph | i-Pr | H | H | H | H | Me | Me |
| CH₂-4-OCH₃—Ph | Pr | Me | Me | H | H | H | H |
| CH₂-4-OCH₃—Ph | Pr | H | H | Me | Me | H | H |
| CH₂-4-OCH₃—Ph | Pr | H | H | H | H | Me | Me |
| CH₂-4-OCH₃—Ph | Bu | Me | Me | H | H | H | H |
| CH₂-4-OCH₃—Ph | Bu | H | H | Me | Me | H | H |
| CH₂-4-OCH₃—Ph | Bu | H | H | H | H | Me | Me |
| CH₂-2-F—Ph | H | Me | Me | H | H | H | H |
| CH₂-2-F—Ph | H | H | H | Me | Me | H | H |
| CH₂-2-F—Ph | H | H | H | H | H | Me | Me |
| CH₂-2-F—Ph | Me | Me | Me | H | H | H | H |
| CH₂-2-F—Ph | Me | H | H | Me | Me | H | H |
| CH₂-2-F—Ph | Me | H | H | H | H | Me | Me |
| CH₂-2-F—Ph | Et | Me | Me | H | H | H | H |
| CH₂-2-F—Ph | Et | H | H | Me | Me | H | H |
| CH₂-2-F—Ph | Et | H | H | H | H | Me | Me |
| CH₂-2-F—Ph | i-Pr | Me | Me | H | H | H | H |
| CH₂-2-F—Ph | i-Pr | H | H | Me | Me | H | H |
| CH₂-2-F—Ph | i-Pr | H | H | H | H | Me | Me |
| CH₂-2-F—Ph | Pr | Me | Me | H | H | H | H |
| CH₂-2-F—Ph | Pr | H | H | Me | Me | H | H |
| CH₂-2-F—Ph | Pr | H | H | H | H | Me | Me |
| CH₂-2-F—Ph | Bu | Me | Me | H | H | H | H |
| CH₂-2-F—Ph | Bu | H | H | Me | Me | H | H |
| CH₂-2-F—Ph | Bu | H | H | H | H | Me | Me |

TABLE L77

| R¹ | R² | R³ | R⁴ | R⁵ | R⁶ | R⁷ | R⁸ |
|---|---|---|---|---|---|---|---|
| CH₂-3-F—Ph | H | Me | Me | H | H | H | H |
| CH₂-3-F—Ph | H | H | H | Me | Me | H | H |
| CH₂-3-F—Ph | H | H | H | H | H | Me | Me |
| CH₂-3-F—Ph | Me | Me | Me | H | H | H | H |
| CH₂-3-F—Ph | Me | H | H | Me | Me | H | H |
| CH₂-3-F—Ph | Me | H | H | H | H | Me | Me |
| CH₂-3-F—Ph | Et | Me | Me | H | H | H | H |
| CH₂-3-F—Ph | Et | H | H | Me | Me | H | H |
| CH₂-3-F—Ph | Et | H | H | H | H | Me | Me |
| CH₂-3-F—Ph | i-Pr | Me | Me | H | H | H | H |
| CH₂-3-F—Ph | i-Pr | H | H | Me | Me | H | H |
| CH₂-3-F—Ph | i-Pr | H | H | H | H | Me | Me |
| CH₂-3-F—Ph | Pr | Me | Me | H | H | H | H |
| CH₂-3-F—Ph | Pr | H | H | Me | Me | H | H |
| CH₂-3-F—Ph | Pr | H | H | H | H | Me | Me |
| CH₂-3-F—Ph | Bu | Me | Me | H | H | H | H |
| CH₂-3-F—Ph | Bu | H | H | Me | Me | H | H |
| CH₂-3-F—Ph | Bu | H | H | H | H | Me | Me |
| CH₂-4-F—Ph | H | Me | Me | H | H | H | H |
| CH₂-4-F—Ph | H | H | H | Me | Me | H | H |
| CH₂-4-F—Ph | H | H | H | H | H | Me | Me |
| CH₂-4-F—Ph | Me | Me | Me | H | H | H | H |
| CH₂-4-F—Ph | Me | H | H | Me | Me | H | H |
| CH₂-4-F—Ph | Me | H | H | H | H | Me | Me |
| CH₂-4-F—Ph | Et | Me | Me | H | H | H | H |
| CH₂-4-F—Ph | Et | H | H | Me | Me | H | H |
| CH₂-4-F—Ph | Et | H | H | H | H | Me | Me |
| CH₂-4-F—Ph | i-Pr | Me | Me | H | H | H | H |
| CH₂-4-F—Ph | i-Pr | H | H | Me | Me | H | H |
| CH₂-4-F—Ph | i-Pr | H | H | H | H | Me | Me |
| CH₂-4-F—Ph | Pr | Me | Me | H | H | H | H |
| CH₂-4-F—Ph | Pr | H | H | Me | Me | H | H |
| CH₂-4-F—Ph | Pr | H | H | H | H | Me | Me |
| CH₂-4-F—Ph | Bu | Me | Me | H | H | H | H |
| CH₂-4-F—Ph | Bu | H | H | Me | Me | H | H |
| CH₂-4-F—Ph | Bu | H | H | H | H | Me | Me |

TABLE L78

| R¹ | R² | R³ | R⁴ | R⁵ | R⁶ | R⁷ | R⁸ |
|---|---|---|---|---|---|---|---|
| CH₂-2-Cl—Ph | H | Me | Me | H | H | H | H |
| CH₂-2-Cl—Ph | H | H | H | Me | Me | H | H |
| CH₂-2-Cl—Ph | H | H | H | H | H | Me | Me |
| CH₂-2-Cl—Ph | Me | Me | Me | H | H | H | H |
| CH₂-2-Cl—Ph | Me | H | H | Me | Me | H | H |
| CH₂-2-Cl—Ph | Me | H | H | H | H | Me | Me |
| CH₂-2-Cl—Ph | Et | Me | Me | H | H | H | H |
| CH₂-2-Cl—Ph | Et | H | H | Me | Me | H | H |
| CH₂-2-Cl—Ph | Et | H | H | H | H | Me | Me |
| CH₂-2-Cl—Ph | i-Pr | Me | Me | H | H | H | H |
| CH₂-2-Cl—Ph | i-Pr | H | H | Me | Me | H | H |
| CH₂-2-Cl—Ph | i-Pr | H | H | H | H | Me | Me |
| CH₂-2-Cl—Ph | Pr | Me | Me | H | H | H | H |
| CH₂-2-Cl—Ph | Pr | H | H | Me | Me | H | H |
| CH₂-2-Cl—Ph | Pr | H | H | H | H | Me | Me |
| CH₂-2-Cl—Ph | Bu | Me | Me | H | H | H | H |
| CH₂-2-Cl—Ph | Bu | H | H | Me | Me | H | H |
| CH₂-2-Cl—Ph | Bu | H | H | H | H | Me | Me |
| CH₂-3-Cl—Ph | H | Me | Me | H | H | H | H |
| CH₂-3-Cl—Ph | H | H | H | Me | Me | H | H |
| CH₂-3-Cl—Ph | H | H | H | H | H | Me | Me |
| CH₂-3-Cl—Ph | Me | Me | Me | H | H | H | H |
| CH₂-3-Cl—Ph | Me | H | H | Me | Me | H | H |
| CH₂-3-Cl—Ph | Me | H | H | H | H | Me | Me |
| CH₂-3-Cl—Ph | Et | Me | Me | H | H | H | H |
| CH₂-3-Cl—Ph | Et | H | H | Me | Me | H | H |
| CH₂-3-Cl—Ph | Et | H | H | H | H | Me | Me |
| CH₂-3-Cl—Ph | i-Pr | Me | Me | H | H | H | H |
| CH₂-3-Cl—Ph | i-Pr | H | H | Ms | Me | H | H |
| CH₂-3-Cl—Ph | i-Pr | H | H | H | H | Me | Me |
| CH₂-3-Cl—Ph | Pr | Me | Me | H | H | H | H |
| CH₂-3-Cl—Ph | Pr | H | H | Me | Me | H | H |
| CH₂-3-Cl—Ph | Pr | H | H | H | H | Me | Me |
| CH₂-3-Cl—Ph | Bu | Me | Me | H | H | H | H |
| CH₂-3-Cl—Ph | Bu | H | H | Me | Me | H | H |
| CH₂-3-Cl—Ph | Bu | H | H | H | H | Me | Me |

TABLE L79

| R¹ | R² | R³ | R⁴ | R⁵ | R⁶ | R⁷ | R⁸ |
|---|---|---|---|---|---|---|---|
| CH₂-4-Cl—Ph | H | Me | Me | H | H | H | H |
| CH₂-4-Cl—Ph | H | H | H | Me | Me | H | H |
| CH₂-4-Cl—Ph | H | H | H | H | H | Me | Me |
| CH₂-4-Cl—Ph | Me | Me | Me | H | H | H | H |
| CH₂-4-Cl—Ph | Me | H | H | Me | Me | H | H |
| CH₂-4-Cl—Ph | Me | H | H | H | H | Me | Me |
| CH₂-4-Cl—Ph | Et | Me | Me | H | H | H | H |
| CH₂-4-Cl—Ph | Et | H | H | Me | Me | H | H |
| CH₂-4-Cl—Ph | Et | H | H | H | H | Me | Me |
| CH₂-4-Cl—Ph | i-Pr | Me | Me | H | H | H | H |
| CH₂-4-Cl—Ph | i-Pr | H | H | Me | Me | H | H |
| CH₂-4-Cl—Ph | i-Pr | H | H | H | H | Me | Me |
| CH₂-4-Cl—Ph | Pr | Me | Me | H | H | H | H |
| CH₂-4-Cl—Ph | Pr | H | H | Me | Me | H | H |
| CH₂-4-Cl—Ph | Pr | H | H | H | H | Me | Me |
| CH₂-4-Cl—Ph | Bu | Me | Me | H | H | H | H |
| CH₂-4-Cl—Ph | Bu | H | H | Me | Me | H | H |
| CH₂-4-Cl—Ph | Bu | H | H | H | H | Me | Me |
| CH₂-2-Br—Ph | H | Me | Me | H | H | H | H |
| CH₂-2-Br—Ph | H | H | H | Me | Me | H | H |
| CH₂-2-Br—Ph | H | H | H | H | H | Me | Me |
| CH₂-2-Br—Ph | Me | Me | Me | H | H | H | H |
| CH₂-2-Br—Ph | Me | H | H | Me | Me | H | H |
| CH₂-2-Br—Ph | Me | H | H | H | H | Me | Me |
| CH₂-2-Br—Ph | Et | Me | Me | H | H | H | H |
| CH₂-2-Br—Ph | Et | H | H | Me | Me | H | H |
| CH₂-2-Br—Ph | Et | H | H | H | H | Me | Me |
| CH₂-2-Br—Ph | i-Pr | Me | Me | H | H | H | H |
| CH₂-2-Br—Ph | i-Pr | H | H | Me | Me | H | H |
| CH₂-2-Br—Ph | i-Pr | H | H | H | H | Me | Me |
| CH₂-2-Br—Ph | Pr | Me | Me | H | H | H | H |
| CH₂-2-Br—Ph | Pr | H | H | Me | Me | H | H |
| CH₂-2-Br—Ph | Pr | H | H | H | H | Me | Me |
| CH₂-2-Br—Ph | Bu | Me | Me | H | H | H | H |
| CH₂-2-Br—Ph | Bu | H | H | Me | Me | H | H |
| CH₂-2-Br—Ph | Bu | H | H | H | H | Me | Me |

TABLE L80

| R¹ | R² | R³ | R⁴ | R⁵ | R⁶ | R⁷ | R⁸ |
|---|---|---|---|---|---|---|---|
| CH₂-3-Br—Ph | H | Me | Me | H | H | H | H |
| CH₂-3-Br—Ph | H | H | H | Me | Me | H | H |
| CH₂-3-Br—Ph | H | H | H | H | H | Me | Me |
| CH₂-3-Br—Ph | Me | Me | Me | H | H | H | H |
| CH₂-3-Br—Ph | Me | H | H | Me | Me | H | H |
| CH₂-3-Br—Ph | Me | H | H | H | H | Me | Me |
| CH₂-3-Br—Ph | Et | Me | Me | H | H | H | H |
| CH₂-3-Br—Ph | Et | H | H | Me | Me | H | H |
| CH₂-3-Br—Ph | Et | H | H | H | H | Me | Me |
| CH₂-3-Br—Ph | i-Pr | Me | Me | H | H | H | H |
| CH₂-3-Br—Ph | i-Pr | H | H | Me | Me | H | H |
| CH₂-3-Br—Ph | i-Pr | H | H | H | H | Me | Me |
| CH₂-3-Br—Ph | Pr | Me | Me | H | H | H | H |
| CH₂-3-Br—Ph | Pr | H | H | Me | Me | H | H |
| CH₂-3-Br—Ph | Pr | H | H | H | H | Me | Me |
| CH₂-3-Br—Ph | Bu | Me | Me | H | H | H | H |
| CH₂-3-Br—Ph | Bu | H | H | Me | Me | H | H |
| CH₂-3-Br—Ph | Bu | H | H | H | H | Me | Me |
| CH₂-4-Br—Ph | H | Me | Me | H | H | H | H |
| CH₂-4-Br—Ph | H | H | H | Me | Me | H | H |
| CH₂-4-Br—Ph | H | H | H | H | H | Me | Me |
| CH₂-4-Br—Ph | Me | Me | Me | H | H | H | H |
| CH₂-4-Br—Ph | Me | H | H | Me | Me | H | H |
| CH₂-4-Br—Ph | Me | H | H | H | H | Me | Me |
| CH₂-4-Br—Ph | Et | Me | Me | H | H | H | H |
| CH₂-4-Br—Ph | Et | H | H | Me | Me | H | H |
| CH₂-4-Br—Ph | Et | H | H | H | H | Me | Me |
| CH₂-4-Br—Ph | i-Pr | Me | Me | H | H | H | H |
| CH₂-4-Br—Ph | i-Pr | H | H | Me | Me | H | H |
| CH₂-4-Br—Ph | i-Pr | H | H | H | H | Me | Me |
| CH₂-4-Br—Ph | Pr | Me | Me | H | H | H | H |
| CH₂-4-Br—Ph | Pr | H | H | Me | Me | H | H |
| CH₂-4-Br—Ph | Pr | H | H | H | H | Me | Me |
| CH₂-4-Br—Ph | Bu | Me | Me | H | H | H | H |
| CH₂-4-Br—Ph | Bu | H | H | Me | Me | H | H |
| CH₂-4-Br—Ph | Bu | H | H | H | H | Me | Me |

TABLE L81

| R¹ | R² | R³ | R⁴ | R⁵ | R⁶ | R⁷ | R⁸ |
|---|---|---|---|---|---|---|---|
| CH₂-2-CF₃—Ph | H | Me | Me | H | H | H | H |
| CH₂-2-CF₃—Ph | H | H | H | Me | Me | H | H |
| CH₂-2-CF₃—Ph | H | H | H | H | H | Me | Me |
| CH₂-2-CF₃—Ph | Me | Me | Me | H | H | H | H |
| CH₂-2-CF₃—Ph | Me | H | H | Me | Me | H | H |
| CH₂-2-CF₃—Ph | Me | H | H | H | H | Me | Me |
| CH₂-2-CF₃—Ph | Et | Me | Me | H | H | H | H |
| CH₂-2-CF₃—Ph | Et | H | H | Me | Me | H | H |
| CH₂-2-CF₃—Ph | Et | H | H | H | H | Me | Me |
| CH₂-2-CF₃—Ph | i-Pr | Me | Me | H | H | H | H |
| CH₂-2-CF₃—Ph | i-Pr | H | H | Me | Me | H | H |
| CH₂-2-CF₃—Ph | i-Pr | H | H | H | H | Me | Me |
| CH₂-2-CF₃—Ph | Pr | Me | Me | H | H | H | H |
| CH₂-2-CF₃—Ph | Pr | H | H | Me | Me | H | H |
| CH₂-2-CF₃—Ph | Pr | H | H | H | H | Me | Me |
| CH₂-2-CF₃—Ph | Bu | Me | Me | H | H | H | H |
| CH₂-2-CF₃—Ph | Bu | H | H | Me | Me | H | H |
| CH₂-2-CF₃—Ph | Bu | H | H | H | H | Me | Me |
| CH₂-3-CF₃—Ph | H | Me | Me | H | H | H | H |
| CH₂-3-CF₃—Ph | H | H | H | Me | Me | H | H |
| CH₂-3-CF₃—Ph | H | H | H | H | H | Me | Me |
| CH₂-3-CF₃—Ph | Me | Me | Me | H | H | H | H |
| CH₂-3-CF₃—Ph | Me | H | H | Me | Me | H | H |
| CH₂-3-CF₃—Ph | Me | H | H | H | H | Me | Me |
| CH₂-3-CF₃—Ph | Et | Me | Me | H | H | H | H |
| CH₂-3-CF₃—Ph | Et | H | H | Me | Me | H | H |
| CH₂-3-CF₃—Ph | Et | H | H | H | H | Me | Me |
| CH₂-3-CF₃—Ph | i-Pr | Me | Me | H | H | H | H |
| CH₂-3-CF₃—Ph | i-Pr | H | H | Me | Me | H | H |
| CH₂-3-CF₃—Ph | i-Pr | H | H | H | H | Me | Me |
| CH₂-3-CF₃—Ph | Pr | Me | Me | H | H | H | H |
| CH₂-3-CF₃—Ph | Pr | H | H | Me | Me | H | H |
| CH₂-3-CF₃—Ph | Pr | H | H | H | H | Me | Me |
| CH₂-3-CF₃—Ph | Bu | Me | Me | H | H | H | H |
| CH₂-3-CF₃—Ph | Bu | H | H | Me | Me | H | H |
| CH₂-3-CF₃—Ph | Bu | H | H | H | H | Me | Me |

TABLE L82

| R¹ | R² | R³ | R⁴ | R⁵ | R⁶ | R⁷ | R⁸ |
|---|---|---|---|---|---|---|---|
| CH₂-4-CF₃—Ph | H | Me | Me | H | H | H | H |
| CH₂-4-CF₃—Ph | H | H | H | Me | Me | H | H |
| CH₂-4-CF₃—Ph | H | H | H | H | H | Me | Me |
| CH₂-4-CF₃—Ph | Me | Me | Me | H | H | H | H |
| CH₂-4-CF₃—Ph | Me | H | H | Me | Me | H | H |
| CH₂-4-CF₃—Ph | Me | H | H | H | H | Me | Me |
| CH₂-4-CF₃—Ph | Et | Me | Me | H | H | H | H |
| CH₂-4-CF₃—Ph | Et | H | H | Me | Me | H | H |
| CH₂-4-CF₃—Ph | Et | H | H | H | H | Me | Me |
| CH₂-4-CF₃—Ph | i-Pr | Me | Me | H | H | H | H |
| CH₂-4-CF₃—Ph | i-Pr | H | H | Me | Me | H | H |
| CH₂-4-CF₃—Ph | i-Pr | H | H | H | H | Me | Me |
| CH₂-4-CF₃—Ph | Pr | Me | Me | H | H | H | H |
| CH₂-4-CF₃—Ph | Pr | H | H | Me | Me | H | H |
| CH₂-4-CF₃—Ph | Pr | H | H | H | H | Me | Me |
| CH₂-4-CF₃—Ph | Bu | Me | Me | H | H | H | H |
| CH₂-4-CF₃—Ph | Bu | H | H | Me | Me | H | H |
| CH₂-4-CF₃—Ph | Bu | H | H | H | H | Me | Me |
| CH₂-2-OCF₃—Ph | H | Me | Me | H | H | H | H |
| CH₂-2-OCF₃—Ph | H | H | H | Me | Me | H | H |
| CH₂-2-OCF₃—Ph | H | H | H | H | H | Me | Me |
| CH₂-2-OCF₃—Ph | Me | Me | Me | H | H | H | H |
| CH₂-2-OCF₃—Ph | Me | H | H | Me | Me | H | H |
| CH₂-2-OCF₃—Ph | Me | H | H | H | H | Me | Me |
| CH₂-2-OCF₃—Ph | Et | Me | Me | H | H | H | H |
| CH₂-2-OCF₃—Ph | Et | H | H | Me | Me | H | H |
| CH₂-2-OCF₃—Ph | Et | H | H | H | H | Me | Me |
| CH₂-2-OCF₃—Ph | i-Pr | Me | Me | H | H | H | H |
| CH₂-2-OCF₃—Ph | i-Pr | H | H | Me | Me | H | H |
| CH₂-2-OCF₃—Ph | i-Pr | H | H | H | H | Me | Me |
| CH₂-2-OCF₃—Ph | Pr | Me | Me | H | H | H | H |
| CH₂-2-OCF₃—Ph | Pr | H | H | Me | Me | H | H |
| CH₂-2-OCF₃—Ph | Pr | H | H | H | H | Me | Me |
| CH₂-2-OCF₃—Ph | Bu | Me | Me | H | H | H | H |
| CH₂-2-OCF₃—Ph | Bu | H | H | Me | Me | H | H |
| CH₂-2-OCF₃—Ph | Bu | H | H | H | H | Me | Me |

TABLE L83

| R¹ | R² | R³ | R⁴ | R⁵ | R⁶ | R⁷ | R⁸ |
|---|---|---|---|---|---|---|---|
| CH₂-3-OCF₃—Ph | H | Me | Me | H | H | H | H |
| CH₂-3-OCF₃—Ph | H | H | H | Me | Me | H | H |
| CH₂-3-OCF₃—Ph | H | H | H | H | H | Me | Me |
| CH₂-3-OCF₃—Ph | Me | Me | Me | H | H | H | H |
| CH₂-3-OCF₃—Ph | Me | H | H | Me | Me | H | H |
| CH₂-3-OCF₃—Ph | Me | H | H | H | H | Me | Me |
| CH₂-3-OCF₃—Ph | Et | Me | Me | H | H | H | H |
| CH₂-3-OCF₃—Ph | Et | H | H | Me | Me | H | H |
| CH₂-3-OCF₃—Ph | Et | H | H | H | H | Me | Me |
| CH₂-3-OCF₃—Ph | i-Pr | Me | Me | H | H | H | H |
| CH₂-3-OCF₃—Ph | i-Pr | H | H | Me | Me | H | H |
| CH₂-3-OCF₃—Ph | i-Pr | H | H | H | H | Me | Me |
| CH₂-3-OCF₃—Ph | Pr | Me | Me | H | H | H | H |
| CH₂-3-OCF₃—Ph | Pr | H | H | Me | Me | H | H |
| CH₂-3-OCF₃—Ph | Pr | H | H | H | H | Me | Me |
| CH₂-3-OCF₃—Ph | Bu | Me | Me | H | H | H | H |
| CH₂-3-OCF₃—Ph | Bu | H | H | Me | Me | H | H |
| CH₂-3-OCF₃—Ph | Bu | H | H | H | H | Me | Me |
| CH₂-4-OCF₃—Ph | H | Me | Me | H | H | H | H |
| CH₂-4-OCF₃—Ph | H | H | H | Me | Me | H | H |
| CH₂-4-OCF₃—Ph | H | H | H | H | H | Me | Me |
| CH₂-4-OCF₃—Ph | Me | Me | Me | H | H | H | H |
| CH₂-4-OCF₃—Ph | Me | H | H | Me | Me | H | H |
| CH₂-4-OCF₃—Ph | Me | H | H | H | H | Me | Me |
| CH₂-4-OCF₃—Ph | Et | Me | Me | H | H | H | H |
| CH₂-4-OCF₃—Ph | Et | H | H | Me | Me | H | H |
| CH₂-4-OCF₃—Ph | Et | H | H | H | H | Me | Me |
| CH₂-4-OCF₃—Ph | i-Pr | Me | Me | H | H | H | H |
| CH₂-4-OCF₃—Ph | i-Pr | H | H | Me | Me | H | H |
| CH₂-4-OCF₃—Ph | i-Pr | H | H | H | H | Me | Me |
| CH₂-4-OCF₃—Ph | Pr | Me | Me | H | H | H | H |
| CH₂-4-OCF₃—Ph | Pr | H | H | Me | Me | H | H |
| CH₂-4-OCF₃—Ph | Pr | H | H | H | H | Me | Me |
| CH₂-4-OCF₃—Ph | Bu | Me | Me | H | H | H | H |
| CH₂-4-OCF₃—Ph | Bu | H | H | Me | Me | H | H |
| CH₂-4-OCF₃—Ph | Bu | H | H | H | H | Me | Me |

TABLE L84

| R¹ | R² | R³ | R⁴ | R⁵ | R⁶ | R⁷ | R⁸ |
|---|---|---|---|---|---|---|---|
| CH₂-4-Ph-Ph | H | Me | Me | H | H | H | H |
| CH₂-4-Ph-Ph | H | H | H | Me | Me | H | H |
| CH₂-4-Ph-Ph | H | H | H | H | H | Me | Me |
| CH₂-4-Ph-Ph | Me | Me | Me | H | H | H | H |
| CH₂-4-Ph-Ph | Me | H | H | Me | Me | H | H |
| CH₂-4-Ph-Ph | Me | H | H | H | H | Me | Me |
| CH₂-4-Ph-Ph | Et | Me | Me | H | H | H | H |
| CH₂-4-Ph-Ph | Et | H | H | Me | Me | H | H |
| CH₂-4-Ph-Ph | Et | H | H | H | H | Me | Me |
| CH₂-4-Ph-Ph | i-Pr | Me | Me | H | H | H | H |
| CH₂-4-Ph-Ph | i-Pr | H | H | Me | Me | H | H |
| CH₂-4-Ph-Ph | i-Pr | H | H | H | H | Me | Me |
| CH₂-4-Ph-Ph | Pr | Me | Me | H | H | H | H |
| CH₂-4-Ph-Ph | Pr | H | H | Me | Me | H | H |
| CH₂-4-Ph-Ph | Pr | H | H | H | H | Me | Me |
| CH₂-4-Ph-Ph | Bu | Me | Me | H | H | H | H |
| CH₂-4-Ph-Ph | Bu | H | H | Me | Me | H | H |
| CH₂-4-Ph-Ph | Bu | H | H | H | H | Me | Me |
| CH₂-3-Ph-Ph | H | Me | Me | H | H | H | H |
| CH₂-3-Ph-Ph | H | H | H | Me | Me | H | H |
| CH₂-3-Ph-Ph | H | H | H | H | H | Me | Me |
| CH₂-3-Ph-Ph | Me | Me | Me | H | H | H | H |
| CH₂-3-Ph-Ph | Me | H | H | Me | Me | H | H |
| CH₂-3-Ph-Ph | Me | H | H | H | H | Me | Me |
| CH₂-3-Ph-Ph | Et | Me | Me | H | H | H | H |
| CH₂-3-Ph-Ph | Et | H | H | Me | Me | H | H |
| CH₂-3-Ph-Ph | Et | H | H | H | H | Me | Me |
| CH₂-3-Ph-Ph | i-Pr | Me | Me | H | H | H | H |
| CH₂-3-Ph-Ph | i-Pr | H | H | Me | Me | H | H |
| CH₂-3-Ph-Ph | i-Pr | H | H | H | H | Me | Me |
| CH₂-3-Ph-Ph | Pr | Me | Me | H | H | H | H |
| CH₂-3-Ph-Ph | Pr | H | H | Me | Me | H | H |
| CH₂-3-Ph-Ph | Pr | H | H | H | H | Me | Me |
| CH₂-3-Ph-Ph | Bu | Me | Me | H | H | H | H |
| CH₂-3-Ph-Ph | Bu | H | H | Me | Me | H | H |
| CH₂-3-Ph-Ph | Bu | H | H | H | H | Me | Me |

TABLE L85

| R¹ | R² | R³ | R⁴ | R⁵ | R⁶ | R⁷ | R⁸ |
|---|---|---|---|---|---|---|---|
| CH₂-4-Ph-Ph | H | Me | Me | H | H | H | H |
| CH₂-4-Ph-Ph | H | H | H | Me | Me | H | H |
| CH₂-4-Ph-Ph | H | H | H | H | H | Me | Me |
| CH₂-4-Ph-Ph | Me | Me | Me | H | H | H | H |
| CH₂-4-Ph-Ph | Me | H | H | Me | Me | H | H |
| CH₂-4-Ph-Ph | Me | H | H | H | H | Me | Me |
| CH₂-4-Ph-Ph | Et | Me | Me | H | H | H | H |
| CH₂-4-Ph-Ph | Et | H | H | Me | Me | H | H |
| CH₂-4-Ph-Ph | Et | H | H | H | H | Me | Me |
| CH₂-4-Ph-Ph | i-Pr | Me | Me | H | H | H | H |
| CH₂-4-Ph-Ph | i-Pr | H | H | Me | Me | H | H |
| CH₂-4-Ph-Ph | i-Pr | H | H | H | H | Me | Me |
| CH₂-4-Ph-Ph | Pr | Me | Me | H | H | H | H |
| CH₂-4-Ph-Ph | Pr | H | H | Me | Me | H | H |
| CH₂-4-Ph-Ph | Pr | H | H | H | H | Me | Me |
| CH₂-4-Ph-Ph | Bu | Me | Me | H | H | H | H |
| CH₂-4-Ph-Ph | Bu | H | H | Me | Me | H | H |
| CH₂-4-Ph-Ph | Bu | H | H | H | H | Me | Me |
| CH₂-2-OPh-Ph | H | Me | Me | H | H | H | H |
| CH₂-2-OPh-Ph | H | H | H | Me | Me | H | H |
| CH₂-2-OPh-Ph | H | H | H | H | H | Me | Me |
| CH₂-2-OPh-Ph | Me | Me | Me | H | H | H | H |
| CH₂-2-OPh-Ph | Me | H | H | Me | Me | H | H |
| CH₂-2-OPh-Ph | Me | H | H | H | H | Me | Me |
| CH₂-2-OPh-Ph | Et | Me | Me | H | H | H | H |
| CH₂-2-OPh-Ph | Et | H | H | Me | Me | H | H |
| CH₂-2-OPh-Ph | Et | H | H | H | H | Me | Me |
| CH₂-2-OPh-Ph | i-Pr | Me | Me | H | H | H | H |
| CH₂-2-OPh-Ph | i-Pr | H | H | Me | Me | H | H |
| CH₂-2-OPh-Ph | i-Pr | H | H | H | H | Me | Me |
| CH₂-2-OPh-Ph | Pr | Me | Me | H | H | H | H |
| CH₂-2-OPh-Ph | Pr | H | H | Me | Me | H | H |
| CH₂-2-OPh-Ph | Pr | H | H | H | H | Me | Me |
| CH₂-2-OPh-Ph | Bu | Me | Me | H | H | H | H |
| CH₂-2-OPh-Ph | Bu | H | H | Me | Me | H | H |
| CH₂-2-OPh-Ph | Bu | H | H | H | H | Me | Me |

TABLE L86

| R¹ | R² | R³ | R⁴ | R⁵ | R⁶ | R⁷ | R⁸ |
|---|---|---|---|---|---|---|---|
| CH₂-3-OPh-Ph | H | Me | Me | H | H | H | H |
| CH₂-3-OPh-Ph | H | H | H | Me | Me | H | H |
| CH₂-3-OPh-Ph | H | H | H | H | H | Me | Me |
| CH₂-3-OPh-Ph | Me | Me | Me | H | H | H | H |
| CH₂-3-OPh-Ph | Me | H | H | Me | Me | H | H |
| CH₂-3-OPh-Ph | Me | H | H | H | H | Me | Me |
| CH₂-3-OPh-Ph | Et | Me | Me | H | H | H | H |
| CH₂-3-OPh-Ph | Et | H | H | Me | Me | H | H |
| CH₂-3-OPh-Ph | Et | H | H | H | H | Me | Me |
| CH₂-3-OPh-Ph | i-Pr | Me | Me | H | H | H | H |
| CH₂-3-OPh-Ph | i-Pr | H | H | Me | Me | H | H |
| CH₂-3-OPh-Ph | i-Pr | H | H | H | H | Me | Me |
| CH₂-3-OPh-Ph | Pr | Me | Me | H | H | H | H |
| CH₂-3-OPh-Ph | Pr | H | H | Me | Me | H | H |
| CH₂-3-OPh-Ph | Pr | H | H | H | H | Me | Me |
| CH₂-3-OPh-Ph | Bu | Me | Me | H | H | H | H |
| CH₂-3-OPh-Ph | Bu | H | H | Me | Me | H | H |
| CH₂-3-OPh-Ph | Bu | H | H | H | H | Me | Me |
| CH₂-4-OPh-Ph | H | Me | Me | H | H | H | H |
| CH₂-4-OPh-Ph | H | H | H | Me | Me | H | H |
| CH₂-4-OPh-Ph | H | H | H | H | H | Me | Me |
| CH₂-4-OPh-Ph | Me | Me | Me | H | H | H | H |
| CH₂-4-OPh-Ph | Me | H | H | Me | Me | H | H |
| CH₂-4-OPh-Ph | Me | H | H | H | H | Me | Me |
| CH₂-4-OPh-Ph | Et | Me | Me | H | H | H | H |
| CH₂-4-OPh-Ph | Et | H | H | Me | Me | H | H |
| CH₂-4-OPh-Ph | Et | H | H | H | | Me | Me |
| CH₂-4-OPh-Ph | i-Pr | Me | Me | H | H | H | H |
| CH₂-4-OPh-Ph | i-Pr | H | H | Me | Me | H | H |
| CH₂-4-OPh-Ph | i-Pr | H | H | H | H | Me | Me |
| CH₂-4-OPh-Ph | Pr | Me | Me | H | H | H | H |
| CH₂-4-OPh-Ph | Pr | H | H | Me | Me | H | H |
| CH₂-4-OPh-Ph | Pr | H | H | H | H | Me | Me |
| CH₂-4-OPh-Ph | Bu | Me | Me | H | H | H | H |
| CH₂-4-OPh-Ph | Bu | H | H | Me | Me | H | H |
| CH₂-4-OPh-Ph | Bu | H | H | H | H | Me | Me |

TABLE L87

| R¹ | R² | R³ | R⁴ | R⁵ | R⁶ | R⁷ | R⁸ |
|---|---|---|---|---|---|---|---|
| CH₂-2-CN-Ph | H | Me | Me | H | H | H | H |
| CH₂-2-CN-Ph | H | H | H | Me | Me | H | H |
| CH₂-2-CN-Ph | H | H | H | H | H | Me | Me |
| CH₂-2-CN-Ph | Me | Me | Me | H | H | H | H |
| CH₂-2-CN-Ph | Me | H | H | Me | Me | H | H |
| CH₂-2-CN-Ph | Me | H | H | H | H | Me | Me |
| CH₂-2-CN-Ph | Et | Me | Me | H | H | H | H |
| CH₂-2-CN-Ph | Et | H | H | Me | Me | H | H |
| CH₂-2-CN-Ph | Et | H | H | H | H | Me | Me |
| CH₂-2-CN-Ph | i-Pr | Me | Me | H | H | H | H |
| CH₂-2-CN-Ph | i-Pr | H | H | Me | Me | H | H |
| CH₂-2-CN-Ph | i-Pr | H | H | H | | Me | Me |
| CH₂-2-CN-Ph | Pr | Me | Me | H | H | H | H |
| CH₂-2-CN-Ph | Pr | H | H | Me | Me | H | H |
| CH₂-2-CN-Ph | Pr | H | H | H | H | Me | Me |
| CH₂-2-CN-Ph | Bu | Me | Me | H | H | H | H |
| CH₂-2-CN-Ph | Bu | H | H | Me | Me | H | H |
| CH₂-2-CN-Ph | Bu | H | H | H | H | Me | Me |
| CH₂-3-CN-Ph | H | Me | Me | H | H | H | H |
| CH₂-3-CN-Ph | H | H | H | Me | Me | H | H |
| CH₂-3-CN-Ph | H | H | H | H | H | Me | Me |
| CH₂-3-CN-Ph | Me | Me | Me | H | H | H | H |
| CH₂-3-CN-Ph | Me | H | H | Me | Me | H | H |
| CH₂-3-CN-Ph | Me | H | H | H | H | Me | Me |
| CH₂-3-CN-Ph | Et | Me | Me | H | H | H | H |
| CH₂-3-CN-Ph | Et | H | H | Me | Me | H | H |
| CH₂-3-CN-Ph | Et | H | H | H | H | Me | Me |
| CH₂-3-CN-Ph | i-Pr | Me | Me | H | H | H | H |
| CH₂-3-CN-Ph | i-Pr | H | H | Me | Me | H | H |
| CH₂-3-CN-Ph | i-Pr | H | H | H | H | Me | Me |
| CH₂-3-CN-Ph | Pr | Me | Me | H | H | H | H |
| CH₂-3-CN-Ph | Pr | H | H | Me | Me | H | H |
| CH₂-3-CN-Ph | Pr | H | H | H | H | Me | Me |
| CH₂-3-CN-Ph | Bu | Me | Me | H | H | H | H |
| CH₂-3-CN-Ph | Bu | H | H | Me | Me | H | H |
| CH₂-3-CN-Ph | Bu | H | H | H | H | Me | Me |

TABLE L88

| R¹ | R² | R³ | R⁴ | R⁵ | R⁶ | R⁷ | R⁸ |
|---|---|---|---|---|---|---|---|
| CH₂-4-CN-Ph | H | Me | Me | H | H | H | H |
| CH₂-4-CN-Ph | H | H | H | Me | Me | H | H |
| CH₂-4-CN-Ph | H | H | H | H | H | Me | Me |
| CH₂-4-CN-Ph | Me | Me | Me | H | H | H | H |
| CH₂-4-CN-Ph | Me | H | H | Me | Me | H | H |
| CH₂-4-CN-Ph | Me | H | H | H | H | Me | Me |
| CH₂-4-CN-Ph | Et | Me | Me | H | H | H | H |
| CH₂-4-CN-Ph | Et | H | H | Me | Me | H | H |
| CH₂-4-CN-Ph | Et | H | H | H | H | Me | Me |
| CH₂-4-CN-Ph | i-Pr | Me | Me | H | H | H | H |
| CH₂-4-CN-Ph | i-Pr | H | H | Me | Me | H | H |
| CH₂-4-CN-Ph | i-Pr | H | H | H | H | Me | Me |
| CH₂-4-CN-Ph | Pr | Me | Me | H | H | H | H |
| CH₂-4-CN-Ph | Pr | H | H | Me | Me | H | H |
| CH₂-4-CN-Ph | Pr | H | H | H | H | Me | Me |
| CH₂-4-CN-Ph | Bu | Me | Me | H | H | H | H |
| CH₂-4-CN-Ph | Bu | H | H | Me | Me | H | H |
| CH₂-4-CN-Ph | Bu | H | H | H | H | Me | Me |
| CH₂-2-NO₂-Ph | H | Me | Me | H | H | H | H |
| CH₂-2-NO₂-Ph | H | H | H | Me | Me | H | H |
| CH₂-2-NO₂-Ph | H | H | H | H | H | Me | Me |
| CH₂-2-NO₂-Ph | Me | Me | Me | H | H | H | H |
| CH₂-2-NO₂-Ph | Me | H | H | Me | Me | H | H |
| CH₂-2-NO₂-Ph | Me | H | H | H | H | Me | Me |
| CH₂-2-NO₂-Ph | Et | Me | Me | H | H | H | H |
| CH₂-2-NO₂-Ph | Et | H | H | Me | Me | H | H |
| CH₂-2-NO₂-Ph | Et | H | H | H | H | Me | Me |
| CH₂-2-NO₂-Ph | i-Pr | Me | Me | H | H | H | H |
| CH₂-2-NO₂-Ph | i-Pr | H | H | Me | Me | H | H |
| CH₂-2-NO₂-Ph | i-Pr | H | H | H | H | Me | Me |
| CH₂-2-NO₂-Ph | Pr | Me | Me | H | H | H | H |
| CH₂-2-NO₂-Ph | Pr | H | H | Me | Me | H | H |
| CH₂-2-NO₂-Ph | Pr | H | H | H | H | Me | Me |
| CH₂-2-NO₂-Ph | Bu | Me | Me | H | H | H | H |
| CH₂-2-NO₂-Ph | Bu | H | H | Me | Me | H | H |
| CH₂-2-NO₂-Ph | Bu | H | H | H | H | Me | Me |

TABLE L89

| R¹ | R² | R³ | R⁴ | R⁵ | R⁶ | R⁷ | R⁸ |
|---|---|---|---|---|---|---|---|
| CH₂-3-NO₂-Ph | H | Me | Me | H | H | H | H |
| CH₂-3-NO₂-Ph | H | H | H | Me | Me | H | H |
| CH₂-3-NO₂-Ph | H | H | H | H | H | Me | Me |
| CH₂-3-NO₂-Ph | Me | Me | Me | H | H | H | H |
| CH₂-3-NO₂-Ph | Me | H | H | Me | Me | H | H |
| CH₂-3-NO₂-Ph | Me | H | H | H | H | Me | Me |
| CH₂-3-NO₂-Ph | Et | Me | Me | H | H | H | H |
| CH₂-3-NO₂-Ph | Et | H | H | Me | Me | H | H |
| CH₂-3-NO₂-Ph | Et | H | H | H | H | Me | Me |
| CH₂-3-NO₂-Ph | i-Pr | Me | Me | H | H | H | H |
| CH₂-3-NO₂-Ph | i-Pr | H | H | Me | Me | H | H |
| CH₂-3-NO₂-Ph | i-Pr | H | H | H | H | Me | Me |
| CH₂-3-NO₂-Ph | Pr | Me | Me | H | H | H | H |
| CH₂-3-NO₂-Ph | Pr | H | H | Me | Me | H | H |
| CH₂-3-NO₂-Ph | Pr | H | H | H | H | Me | Me |
| CH₂-3-NO₂-Ph | Bu | Me | Me | H | H | H | H |
| CH₂-3-NO₂-Ph | Bu | H | H | Me | Me | H | H |
| CH₂-3-NO₂-Ph | Bu | H | H | H | H | Me | Me |
| CH₂-4-NO₂-Ph | H | Me | Me | H | H | H | H |
| CH₂-4-NO₂-Ph | H | H | H | Me | Me | H | H |
| CH₂-4-NO₂-Ph | H | H | H | H | H | Me | Me |
| CH₂-4-NO₂-Ph | Me | Me | Me | H | H | H | H |
| CH₂-4-NO₂-Ph | Me | H | H | Me | Me | H | H |
| CH₂-4-NO₂-Ph | Me | H | H | H | H | Me | Me |
| CH₂-4-NO₂-Ph | Et | Me | Me | H | H | H | H |
| CH₂-4-NO₂-Ph | Et | H | H | Me | Me | H | H |
| CH₂-4-NO₂-Ph | Et | H | H | H | H | Me | Me |
| CH₂-4-NO₂-Ph | i-Pr | Me | Me | H | H | H | H |
| CH₂-4-NO₂-Ph | i-Pr | H | H | Me | Me | H | H |
| CH₂-4-NO₂-Ph | i-Pr | H | H | H | H | Me | Me |
| CH₂-4-NO₂-Ph | Pr | Me | Me | H | H | H | H |
| CH₂-4-NO₂-Ph | Pr | H | H | Me | Me | H | H |
| CH₂-4-NO₂-Ph | Pr | H | H | H | H | Me | Me |
| CH₂-4-NO₂-Ph | Bu | Me | Me | H | H | H | H |
| CH₂-4-NO₂-Ph | Bu | H | H | Me | Me | H | H |
| CH₂-4-NO₂-Ph | Bu | H | H | H | H | Me | Me |

TABLE L90

| R¹ | R² | R³ | R⁴ | R⁵ | R⁶ | R⁷ | R⁸ |
|---|---|---|---|---|---|---|---|
| CH₂-Py2 | H | Me | Me | H | H | H | H |
| CH₂-Py2 | H | H | H | Me | Me | H | H |
| CH₂-Py2 | H | H | H | H | H | Me | Me |
| CH₂-Py2 | Me | Me | Me | H | H | H | H |
| CH₂-Py2 | Me | H | H | Me | Me | H | H |
| CH₂-Py2 | Me | H | H | H | H | Me | Me |
| CH₂-Py2 | Et | Me | Me | H | H | H | H |
| CH₂-Py2 | Et | H | H | Me | Me | H | H |
| CH₂-Py2 | Et | H | H | H | H | Me | Me |
| CH₂-Py2 | i-Pr | Me | Me | H | H | H | H |
| CH₂-Py2 | i-Pr | H | H | Me | Me | H | H |
| CH₂-Py2 | i-Pr | H | H | H | H | Me | Me |
| CH₂-Py2 | Pr | Me | Me | H | H | H | H |
| CH₂-Py2 | Pr | H | H | Me | Me | H | H |
| CH₂-Py2 | Pr | H | H | H | H | Me | Me |
| CH₂-Py2 | Bu | Me | Me | H | H | H | H |
| CH₂-Py2 | Bu | H | H | Me | Me | H | H |
| CH₂-Py2 | Bu | H | H | H | H | Me | Me |
| CH₂-Py3 | H | Me | Me | H | H | H | H |
| CH₂-Py3 | H | H | H | Me | Me | H | H |
| CH₂-Py3 | H | H | H | H | H | Me | Me |
| CH₂-Py3 | Me | Me | Me | H | H | H | H |
| CH₂-Py3 | Me | H | H | Me | Me | H | H |
| CH₂-Py3 | Me | H | H | H | H | Me | Me |
| CH₂-Py3 | Et | Me | Me | H | H | H | H |
| CH₂-Py3 | Et | H | H | Me | Me | H | H |
| CH₂-Py3 | Et | H | H | H | H | Me | Me |
| CH₂-Py3 | i-Pr | Me | Me | H | H | H | H |
| CH₂-Py3 | i-Pr | H | H | Me | Me | H | H |
| CH₂-Py3 | i-Pr | H | H | H | H | Me | Me |
| CH₂-Py3 | Pr | Me | Me | H | H | H | H |
| CH₂-Py3 | Pr | H | H | Me | Me | H | H |
| CH₂-Py3 | Pr | H | H | H | H | Me | Me |
| CH₂-Py3 | Bu | Me | Me | H | H | H | H |
| CH₂-Py3 | Bu | H | H | Me | Me | H | H |
| CH₂-Py3 | Bu | H | H | H | H | Me | Me |

TABLE L91

| R¹ | R² | R³ | R⁴ | R⁵ | R⁶ | R⁷ | R⁸ |
|---|---|---|---|---|---|---|---|
| CH₂-Py4 | H | Me | Me | H | H | H | H |
| CH₂-Py4 | H | H | H | Me | Me | H | H |
| CH₂-Py4 | H | H | H | H | H | Me | Me |
| CH₂-Py4 | Me | Me | Me | H | H | H | H |
| CH₂-Py4 | Me | H | H | Me | Me | H | H |
| CH₂-Py4 | Me | H | H | H | H | Me | Me |
| CH₂-Py4 | Et | Me | Me | H | H | H | H |
| CH₂-Py4 | Et | H | H | Me | Me | H | H |
| CH₂-Py4 | Et | H | H | H | H | Me | Me |
| CH₂-Py4 | i-Pr | Me | Me | H | H | H | H |
| CH₂-Py4 | i-Pr | H | H | Me | Me | H | H |
| CH₂-Py4 | i-Pr | H | H | H | H | Me | Me |
| CH₂-Py4 | Pr | Me | Me | H | H | H | H |
| CH₂-Py4 | Pr | H | H | Me | Me | H | H |
| CH₂-Py4 | Pr | H | H | H | H | Me | Me |
| CH₂-Py4 | Bu | Me | Me | H | H | H | H |
| CH₂-Py4 | Bu | H | H | Me | Me | | H |
| CH₂-Py4 | Bu | H | H | H | H | Me | Me |
| CH₂-5-CF₃-Py2 | H | Me | Me | H | H | H | H |
| CH₂-5-CF₃-Py2 | H | H | H | Me | Me | H | H |
| CH₂-5-CF₃-Py2 | H | H | H | H | H | Me | Me |
| CH₂-5-CF₃-Py2 | Me | Me | Me | H | H | H | H |
| CH₂-5-CF₃-Py2 | Me | H | H | Me | Me | H | H |
| CH₂-5-CF₃-Py2 | Me | H | H | H | H | Me | Me |
| CH₂-5-CF₃-Py2 | Et | Me | Me | H | H | H | H |
| CH₂-5-CF₃-Py2 | Et | H | H | Me | Me | H | H |
| CH₂-5-CF₃-Py2 | Et | H | H | H | H | Me | Me |
| CH₂-5-CF₃-Py2 | i-Pr | Me | Me | H | H | H | H |
| CH₂-5-CF₃-Py2 | i-Pr | H | H | Me | Me | H | H |
| CH₂-5-CF₃-Py2 | i-Pr | H | H | H | H | Me | Me |
| CH₂-5-CF₃-Py2 | Pr | Me | Me | H | H | H | H |
| CH₂-5-CF₃-Py2 | Pr | H | H | Me | Me | H | H |
| CH₂-5-CF₃-Py2 | Pr | H | H | H | H | Me | Me |
| CH₂-5-CF₃-Py2 | Bu | Me | Me | H | H | H | H |
| CH₂-5-CF₃-Py2 | Bu | H | H | Me | Me | H | H |
| CH₂-5-CF₃-Py2 | Bu | H | H | H | H | Me | Me |

TABLE L92

| R¹ | R² | R³ | R⁴ | R⁵ | R⁶ | R⁷ | R⁸ |
|---|---|---|---|---|---|---|---|
| CH₂-6-CF₃-Py3 | H | Me | Me | H | H | H | H |
| CH₂-6-CF₃-Py3 | H | H | H | Me | Me | H | H |
| CH₂-6-CF₃-Py3 | H | H | H | H | H | Me | Me |
| CH₂-6-CF₃-Py3 | Me | Me | Me | H | H | H | H |
| CH₂-6-CF₃-Py3 | Me | H | H | Me | Me | H | H |
| CH₂-6-CF₃-Py3 | Me | H | H | H | H | Me | Me |
| CH₂-6-CF₃-Py3 | Et | Me | Me | H | H | H | H |
| CH₂-6-CF₃-Py3 | Et | H | H | Me | Me | H | H |
| CH₂-6-CF₃-Py3 | Et | H | H | H | H | Me | Me |
| CH₂-6-CF₃-Py3 | i-Pr | Me | Me | H | H | H | H |
| CH₂-6-CF₃-Py3 | i-Pr | H | H | Me | Me | H | H |
| CH₂-6-CF₃-Py3 | i-Pr | H | H | H | H | Me | Me |
| CH₂-6-CF₃-Py3 | Pr | Me | Me | H | H | H | H |
| CH₂-6-CF₃-Py3 | Pr | H | H | Me | Me | H | H |
| CH₂-6-CF₃-Py3 | Pr | H | | H | H | Me | Me |
| CH₂-6-CF₃-Py3 | Bu | Me | Me | H | H | H | H |
| CH₂-6-CF₃-Py3 | Bu | H | H | Me | Me | H | H |
| CH₂-6-CF₃-Py3 | Bu | H | H | H | H | Me | Me |
| CH₂-furan-2-yl | H | Me | Me | H | H | H | H |
| CH₂-furan-2-yl | H | H | H | Me | Me | H | H |
| CH₂-furan-2-yl | H | H | H | H | H | Me | Me |
| CH₂-furan-2-yl | Me | Me | Me | H | H | H | H |
| CH₂-furan-2-yl | Me | H | H | Me | Me | H | H |
| CH₂-furan-2-yl | Me | H | H | H | H | Me | Me |
| CH₂-furan-2-yl | Et | Me | Me | H | H | H | H |
| CH₂-furan-2-yl | Et | H | H | Me | Me | H | H |
| CH₂-furan-2-yl | Et | H | H | H | H | Me | Me |
| CH₂-furan-2-yl | i-Pr | Me | Me | H | H | H | H |
| CH₂-furan-2-yl | i-Pr | H | H | Me | Me | H | H |
| CH₂-furan-2-yl | i-Pr | H | H | H | H | Me | Me |
| CH₂-furan-2-yl | Pr | Me | Me | H | H | H | H |
| CH₂-furan-2-yl | Pr | H | H | Me | Me | H | H |
| CH₂-furan-2-yl | Pr | H | H | H | H | Me | Me |
| CH₂-furan-2-yl | Bu | Me | Me | H | H | H | H |
| CH₂-furan-2-yl | Bu | H | H | Me | Me | H | H |
| CH₂-furan-2-yl | Bu | H | H | H | H | Me | Me |

TABLE L93

| R¹ | R² | R³ | R⁴ | R⁵ | R⁶ | R⁷ | R⁸ |
|---|---|---|---|---|---|---|---|
| CH₂-furan-3-yl | H | Me | Me | H | H | H | H |
| CH₂-furan-3-yl | H | H | H | Me | Me | H | H |
| CH₂-furan-3-yl | H | H | H | H | H | Me | Me |
| CH₂-furan-3-yl | Me | Me | Me | H | H | H | H |
| CH₂-furan-3-yl | Me | H | H | Me | Me | H | H |
| CH₂-furan-3-yl | Me | H | H | H | H | Me | Me |
| CH₂-furan-3-yl | Et | Me | Me | H | H | H | H |
| CH₂-furan-3-yl | Et | H | H | Me | Me | H | H |
| CH₂-furan-3-yl | Et | H | H | H | H | Me | Me |
| CH₂-furan-3-yl | i-Pr | Me | Me | H | H | H | H |
| CH₂-furan-3-yl | i-Pr | H | H | Me | Me | H | H |
| CH₂-furan-3-yl | i-Pr | H | H | H | H | Me | Me |
| CH₂-furan-3-yl | Pr | Me | Me | H | H | H | H |
| CH₂-furan-3-yl | Pr | H | H | Me | Me | H | H |
| CH₂-furan-3-yl | Pr | H | H | H | H | Me | Me |
| CH₂-furan-3-yl | Bu | Me | Me | H | H | H | H |
| CH₂-furan-3-yl | Bu | H | H | Me | Me | H | H |
| CH₂-furan-3-yl | Bu | H | H | H | H | Me | Me |
| CH₂-5-CF₃-furan-2-yl | H | Me | Me | H | H | H | H |
| CH₂-5-CF₃-furan-2-yl | H | H | H | Me | Me | H | H |
| CH₂-5-CF₃-furan-2-yl | H | H | H | H | H | Me | Me |
| CH₂-5-CF₃-furan-2-yl | Me | Me | Me | H | H | H | H |
| CH₂-5-CF₃-furan-2-yl | Me | H | H | Me | Me | H | H |
| CH₂-5-CF₃-furan-2-yl | Me | H | H | H | H | Me | Me |
| CH₂-5-CF₃-furan-2-yl | Et | Me | Me | H | H | H | H |
| CH₂-5-CF₃-furan-2-yl | Et | H | H | Me | Me | H | H |
| CH₂-5-CF₃-furan-2-yl | Et | H | H | H | H | Me | Me |
| CH₂-5-CF₃-furan-2-yl | i-Pr | Me | Me | H | H | H | H |
| CH₂-5-CF₃-furan-2-yl | i-Pr | H | H | Me | Me | H | H |
| CH₂-5-CF₃-furan-2-yl | i-Pr | H | H | H | H | Me | Me |
| CH₂-5-CF₃-furan-2-yl | Pr | Me | Me | H | H | H | H |
| CH₂-5-CF₃-furan-2-yl | Pr | H | H | Me | Me | H | H |
| CH₂-5-CF₃-furan-2-yl | Pr | H | H | H | H | Me | Me |
| CH₂-5-CF₃-furan-2-yl | Bu | Me | Me | H | H | H | H |
| CH₂-5-CE3-furan-2-yl | Bu | H | H | Me | Me | H | H |
| CH₂-5-CF₃-furan-2-yl | Bu | H | H | H | H | Me | Me |

TABLE L94

| R¹ | R² | R³ | R⁴ | R⁵ | R⁶ | R⁷ | R⁸ |
|---|---|---|---|---|---|---|---|
| CH₂-5-CF₃-furan-3-yl | H | Me | Me | H | H | H | H |
| CH₂-5-CF₃-furan-3-yl | H | H | H | Me | Me | H | H |
| CH₂-5-CF₃-furan-3-yl | H | H | H | H | H | Me | Me |
| CH₂-5-CF₃-furan-3-yl | Me | Me | Me | H | H | H | H |
| CH₂-5-CF₃-furan-3-yl | Me | H | H | Me | Me | H | H |
| CH₂-5-CF₃-furan-3-yl | Me | H | H | H | H | Me | Me |
| CH₂-5-CF₃-furan-3-yl | Et | Me | Me | H | H | H | H |
| CH₂-5-CF₃-furan-3-yl | Et | H | H | Me | Me | H | H |
| CH₂-5-CF₃-furan-3-yl | Et | H | H | H | H | Me | Me |
| CH₂-5-CF₃-furan-3-yl | i-Pr | Me | Me | H | H | H | H |
| CH₂-5-CF₃-furan-3-yl | i-Pr | H | H | Me | Me | H | H |
| CH₂-5-CF₃-furan-3-yl | i-Pr | H | H | H | H | Me | Me |
| CH₂-5-CF₃-furan-3-yl | Pr | Me | Me | H | H | H | H |
| CH₂-5-CF₃-furan-3-yl | Pr | H | H | Me | Me | H | H |
| CH₂-5-CF₃-furan-3-yl | Pr | H | H | H | H | Me | Me |
| CH₂-5-CF₃-furan-3-yl | Bu | Me | Me | H | H | H | H |
| CH₂-5-CF₃-furan-3-yl | Bu | H | H | Me | Me | H | H |
| CH₂-5-CF₃-furan-3-yl | Bu | H | H | H | H | Me | Me |
| CH₂-thiophen-2-yl | H | Me | Me | H | H | H | H |
| CH₂-thiophen-2-yl | H | H | H | Me | Me | H | H |
| CH₂-thiophen-2-yl | H | H | H | H | H | Me | Me |
| CH₂-thiophen-2-yl | Me | Me | Me | H | H | H | H |
| CH₂-thiophen-2-yl | Me | H | H | Me | Me | H | H |
| CH₂-thiophen-2-yl | Me | H | H | H | H | Me | Me |
| CH₂-thiophen-2-yl | Et | Me | Me | H | H | H | H |
| CH₂-thiophen-2-yl | Et | H | H | Me | Me | H | H |
| CH₂-thiophen-2-yl | Et | H | H | H | H | Me | Me |
| CH₂-thiophen-2-yl | i-Pr | Me | Me | H | H | H | H |
| CH₂-thiophen-2-yl | i-Pr | H | H | Me | Me | H | H |
| CH₂-thiophen-2-yl | i-Pr | H | H | H | H | Me | Me |
| CH₂-thiophen-2-yl | Pr | Me | Me | H | H | H | H |
| CH₂-thiophen-2-yl | Pr | H | H | Me | Me | H | H |
| CH₂-thiophen-2-yl | Pr | H | H | H | H | Me | Me |
| CH₂-thiophen-2-yl | Bu | Me | Me | H | H | H | H |
| CH₂-thiophen-2-yl | Bu | H | H | Me | Me | H | H |
| CH₂-thiophen-2-yl | Bu | H | H | H | H | Me | Me |

TABLE L95

| R¹ | R² | R³ | R⁴ | R⁵ | R⁶ | R⁷ | R⁸ |
|---|---|---|---|---|---|---|---|
| CH₂-thiophen-3-yl | H | Me | Me | H | H | H | H |
| CH₂-thiophen-3-yl | H | H | H | Me | Me | H | H |
| CH₂-thiophen-3-yl | H | H | H | H | H | Me | Me |
| CH₂-thiophen-3-yl | Me | Me | Me | H | H | H | H |
| CH₂-thiophen-3-yl | Me | H | H | Me | Me | H | H |
| CH₂-thiophen-3-yl | Me | H | H | H | H | Me | Me |
| CH₂-thiophen-3-yl | Et | Me | Me | H | H | H | H |
| CH₂-thiophen-3-yl | Et | H | H | Me | Me | H | H |
| CH₂-thiophen-3-yl | Et | H | H | H | H | Me | Me |
| CH₂-thiophen-3-yl | i-Pr | Me | Me | H | H | H | H |
| CH₂-thiophen-3-yl | i-Pr | H | H | Me | Me | H | H |
| CH₂-thiophen-3-yl | i-Pr | H | H | H | H | Me | Me |
| CH₂-thiophen-3-yl | Pr | Me | Me | H | H | H | H |
| CH₂-thiophen-3-yl | Pr | H | H | Me | Me | H | H |
| CH₂-thiophen-3-yl | Pr | H | H | H | H | Me | Me |
| CH₂-thiophen-3-yl | Bu | Me | Me | H | H | H | H |
| CH₂-thiophen-3-yl | Bu | H | H | Me | Me | H | H |
| CH₂-thiophen-3-yl | Bu | H | H | H | H | Me | Me |
| CH₂-5-CF₃-thiophen-2-yl | H | Me | Me | H | H | H | H |
| CH₂-5-CF₃-thiophen-2-yl | H | H | H | Me | Me | H | H |
| CH₂-5-CF₃-thiophen-2-yl | H | H | H | H | H | Me | Me |
| CH₂-5-CF₃-thiophen-2-yl | Me | Me | Me | H | H | H | H |
| CH₂-5-CF₃-thiophen-2-yl | Me | H | H | Me | Me | H | H |
| CH₂-5-CF₃-thiophen-2-yl | Me | H | H | H | H | Me | Me |
| CH₂-5-CF₃-thiophen-2-yl | Et | Me | Me | H | H | H | H |
| CH₂-5-CF₃-thiophen-2-yl | Et | H | H | Me | Me | H | H |
| CH₂-5-CF₃-thiophen-2-yl | Et | H | H | H | H | Me | Me |
| CH₂-5-CF₃-thiophen-2-yl | i-Pr | Me | Me | H | H | H | H |
| CH₂-5-CF₃-thiophen-2-yl | i-Pr | H | H | Me | Me | H | H |
| CH₂-5-CF₃-thiophen-2-yl | i-Pr | H | H | H | H | Me | Me |
| CH₂-5-CF₃-thiophen-2-yl | Pr | Me | Me | H | H | H | H |
| CH₂-5-CF₃-thiophen-2-yl | Pr | H | H | Me | Me | H | H |
| CH₂-5-CF₃-thiophen-2-yl | Pr | H | H | H | H | Me | Me |
| CH₂-5-CF₃-thiophen-2-yl | Bu | Me | Me | H | H | H | H |
| CH₂-5-CF₃-thiophen-2-yl | Bu | H | H | Me | Me | H | H |
| CH₂-5-CF₃-thiophen-2-yl | Bu | H | H | H | H | Me | Me |

TABLE L96

| R¹ | R² | R³ | R⁴ | R⁵ | R⁶ | R⁷ | R⁸ |
|---|---|---|---|---|---|---|---|
| CH₂-5-CF₃-thiophen-3-yl | H | Me | Me | H | H | H | H |
| CH₂-5-CF₃-thiophen-3-yl | H | H | H | Me | Me | H | H |
| CH₂-5-CF₃-thiophen-3-yl | H | H | H | H | H | Me | Me |
| CH₂-5-CF₃-thiophen-3-yl | Me | Me | Me | H | H | H | H |
| CH₂-5-CF₃-thiophen-3-yl | Me | H | H | Me | Me | H | H |
| CH₂-5-CF₃-thiophen-3-yl | Me | H | H | H | H | Me | Me |
| CH₂-5-CF₃-thiophen-3-yl | Et | Me | Me | H | H | H | H |
| CH₂-5-CF₃-thiophen-3-yl | Et | H | H | Me | Me | H | H |
| CH₂-5-CF₃-thiophen-3-yl | Et | H | H | H | H | Me | Me |
| CH₂-5-CF₃-thiophen-3-yl | i-Pr | Me | Me | H | H | H | H |
| CH₂-5-CF₃-thiophen-3-yl | i-Pr | H | H | Me | Me | H | H |
| CH₂-5-CF₃-thiophen-3-yl | i-Pr | H | H | H | H | Me | Me |
| CH₂-5-CF₃-thiophen-3-yl | Pr | Me | Me | H | H | H | H |
| CH₂-5-CF₃-thiophen-3-yl | Pr | H | H | Me | Me | H | H |
| CH₂-5-CF₃-thiophen-3-yl | Pr | H | H | H | H | Me | Me |
| CH₂-5-CF₃-thiophen-3-yl | Bu | Me | Me | H | H | H | H |
| CH₂-5-CF₃-thiophen-3-yl | Bu | H | H | Me | Me | H | H |
| CH₂-5-CF₃-thiophen-3-yl | Bu | H | H | H | H | Me | Me |
| pyridazin-3-yl | H | Me | Me | H | H | H | H |
| pyridazin-3-yl | H | H | H | Me | Me | H | H |
| pyridazin-3-yl | H | H | H | H | H | Me | Me |
| pyridazin-3-yl | Me | Me | Me | H | H | I- | H |
| pyridazin-3-yl | Me | H | H | Me | Me | H | H |
| pyridazin-3-yl | Me | H | H | H | H | Me | Me |
| pyridazin-3-yl | Et | Me | Me | H | H | H | H |
| pyridazin-3-yl | Et | H | H | Me | Me | H | H |
| pyridazin-3-yl | Et | H | H | H | H | Me | Me |
| pyridazin-3-yl | i-Pr | Me | Me | H | H | H | H |
| pyridazin-3-yl | i-Pr | H | H | Me | Me | H | H |
| pyridazin-3-yl | i-Pr | H | H | H | H | Me | Me |
| pyridazin-3-yl | Pr | Me | Me | H | H | H | H |
| pyridazin-3-yl | Pr | H | H | Me | Me | H | H |
| pyridazin-3-yl | Pr | H | H | H | H | Me | Me |
| pyridazin-3-yl | Bu | Me | Me | H | H | H | H |
| pyridazin-3-yl | Bu | H | H | Me | Me | H | H |
| pyridazin-3-yl | Bu | H | H | H | H | Me | Me |

TABLE L97

| R¹ | R² | R³ | R⁴ | R⁵ | R⁶ | R⁷ | R⁸ |
|---|---|---|---|---|---|---|---|
| pyridazin-4-yl | H | Me | Me | H | H | H | H |
| pyridazin-4-yl | H | H | H | Me | Me | H | H |
| pyridazin-4-yl | H | H | H | H | H | Me | Me |
| pyridazin-4-yl | Me | Me | Me | H | H | H | H |
| pyridazin-4-yl | Me | H | H | Me | Me | H | H |
| pyridazin-4-yl | Me | H | H | H | H | Me | Me |
| pyridazin-4-yl | Et | Me | Me | H | H | H | H |
| pyridazin-4-yl | Et | H | H | Me | Me | H | H |
| pyridazin-4-yl | Et | H | H | H | H | Me | Me |
| pyridazin-4-yl | i-Pr | Me | Me | H | H | H | H |
| pyridazin-4-yl | i-Pr | H | H | Me | Me | H | H |
| pyridazin-4-yl | i-Pr | H | H | H | H | Me | Me |
| pyridazin-4-yl | Pr | Me | Me | H | H | H | H |
| pyridazin-4-yl | Pr | H | H | Me | Me | H | H |
| pyridazin-4-yl | Pr | H | H | H | H | Me | Me |
| pyridazin-4-yl | Bu | Me | Me | H | H | H | H |
| pyridazin-4-yl | Bu | H | H | Me | Me | H | H |
| pyridazin-4-yl | Bu | H | H | H | H | Me | Me |
| pyrimidin-2-yl | H | Me | Me | H | H | H | H |
| pyrimidin-2-yl | H | H | H | Me | Me | H | H |
| pyrimidin-2-yl | H | H | H | H | H | Me | Me |
| pyrimidin-2-yl | Me | Me | Me | H | H | H | H |
| pyrimidin-2-yl | Me | H | H | Me | Me | H | H |
| pyrimidin-2-yl | Me | H | H | H | H | Me | Me |
| pyrimidin-2-yl | Et | Me | Me | H | H | H | H |
| pyrimidin-2-yl | Et | H | H | Me | Me | H | H |
| pyrimidin-2-yl | Et | H | H | H | H | Me | Me |
| pyrimidin-2-yl | i-Pr | Me | Me | H | H | H | H |
| pyrimidin-2-yl | i-Pr | H | H | Me | Me | H | H |
| pyrimidin-2-yl | i-Pr | H | H | H | H | Me | Me |
| pyrimidin-2-yl | Pr | Me | Me | H | H | H | H |
| pyrimidin-2-yl | Pr | H | H | Me | Me | H | H |
| pyrimidin-2-yl | Pr | H | H | H | H | Me | Me |
| pyrimidin-2-yl | Bu | Me | Me | H | H | H | H |
| pyrimidin-2-yl | Bu | H | H | Me | Me | H | H |
| pyrimidin-2-yl | Bu | H | H | H | H | Me | Me |

TABLE L98

| R¹ | R² | R³ | R⁴ | R⁵ | R⁶ | R⁷ | R⁸ |
|---|---|---|---|---|---|---|---|
| pyrimidin-4-yl | H | Me | Me | H | H | H | H |
| pyrimidin-4-yl | H | H | H | Me | Me | H | H |
| pyrimidin-4-yl | H | H | H | H | H | Me | Me |
| pyrimidin-4-yl | Me | Me | Me | H | H | H | H |
| pyrimidin-4-yl | Me | H | H | Me | Me | H | H |
| pyrimidin-4-yl | Me | H | H | H | H | Me | Me |
| pyrimidin-4-yl | Et | Me | Me | H | H | H | H |
| pyrimidin-4-yl | Et | H | H | Me | Me | H | H |
| pyrimidin-4-yl | Et | H | H | H | H | Me | Me |
| pyrimidin-4-yl | i-Pr | Me | Me | H | H | H | H |
| pyrimidin-4-yl | i-Pr | H | H | Me | Me | H | H |
| pyrimidin-4-yl | i-Pr | H | H | H | H | Me | Me |
| pyrimidin-4-yl | Pr | Me | Me | H | H | H | H |
| pyrimidin-4-yl | Pr | H | H | Me | Me | H | H |
| pyrimidin-4-yl | Pr | H | H | H | H | Me | Me |
| pyrimidin-4-yl | Bu | Me | Me | H | H | H | H |
| pyrimidin-4-yl | Bu | H | H | Me | Me | H | H |
| pyrimidin-4-yl | Bu | H | H | H | H | Me | Me |
| pyrimidin-5-yl | H | Me | Me | H | H | H | H |
| pyrimidin-5-yl | H | H | H | Me | Me | H | H |
| pyrimidin-5-yl | H | H | H | H | H | Me | Me |
| pyrimidin-5-yl | Me | Me | Me | H | H | H | H |
| pyrimidin-5-yl | Me | H | H | Me | Me | H | H |
| pyrimidin-5-yl | Me | H | H | H | H | Me | Me |
| pyrimidin-5-yl | Et | Me | Me | H | H | H | H |
| pyrimidin-5-yl | Et | H | H | Me | Me | H | H |
| pyrimidin-5-yl | Et | H | H | H | H | Me | Me |
| pyrimidin-5-yl | i-Pr | Me | Me | H | H | H | H |
| pyrimidin-5-yl | i-Pr | H | H | Me | Me | H | H |
| pyrimidin-5-yl | i-Pr | H | H | H | H | Me | Me |
| pyrimidin-5-yl | Pr | Me | Me | H | H | H | H |
| pyrimidin-5-yl | Pr | H | H | Me | Me | H | H |
| pyrimidin-5-yl | Pr | H | H | H | H | Me | Me |
| pyrimidin-5-yl | Bu | Me | Me | H | H | H | H |
| pyrimidin-5-yl | Bu | H | H | Me | Me | H | H |
| pyrimidin-5-yl | Bu | H | H | H | H | Me | Me |
| pyrazin-2-yl | H | Me | Me | H | H | H | H |
| pyrazin-2-yl | H | H | H | Me | Me | H | H |
| pyrazin-2-yl | H | H | H | H | H | Me | Me |
| pyrazin-2-yl | Me | Me | Me | H | H | H | H |
| pyrazin-2-yl | Me | H | H | Me | Me | H | H |
| pyrazin-2-yl | Me | H | H | H | H | Me | Me |
| pyrazin-2-yl | Et | Me | Me | H | H | H | H |
| pyrazin-2-yl | Et | H | H | Me | Me | H | H |
| pyrazin-2-yl | Et | H | H | H | H | Me | Me |
| pyrazin-2-yl | i-Pr | Me | Me | H | H | H | H |
| pyrazin-2-yl | i -Pr | H | H | Me | Me | H | H |
| pyrazin-2-yl | i-Pr | H | H | H | H | Me | Me |
| pyrazin-2-yl | Pr | Me | Me | H | | H | H |
| pyrazin-2-yl | Pr | H | H | Me | Me | H | H |
| pyrazin-2-yl | Pr | H | H | H | H | Me | Me |
| pyrazin-2-yl | Bu | Me | Me | H | H | H | H |
| pyrazin-2-yl | Bu | H | H | Me | Me | H | H |
| pyrazin-2-yl | Bu | H | H | H | H | Me | Me |

The compound (L-2), wherein R¹² is a methyl group, R¹³ is a hydrogen atom, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX618).

The compound (L-2), wherein R¹² is a hydrogen atom, R¹³ is a methyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX619).

The compound (L-2), wherein R¹² is a hydrogen atom, R¹³ is a hydrogen atom, R¹⁴ is a methyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX620).

The compound (L-2), wherein R¹² is a hydrogen atom, R¹³ is a hydrogen atom, R¹⁴ is a hydrogen atom, R¹⁵ is a methyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX621).

The compound (L-2), wherein R¹² is a hydrogen atom, R¹³ is a hydrogen atom, R¹⁴ is a hydrogen atom, R¹⁵ is a hydrogen atom, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX622).

The compound (L-2), wherein R¹² is a hydrogen atom, R¹³ is a methyl group, R¹⁴ is a methyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX623).

The compound (L-2), wherein R¹² is a hydrogen atom, R¹³ is a methyl group, R¹⁴ is a hydrogen atom, R¹⁵ is a methyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX624).

The compound (L-2), wherein R¹² is a hydrogen atom, R¹³ is a methyl group, R¹⁴ is a hydrogen atom, R¹⁵ is a hydrogen atom, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX625).

The compound (L-2), wherein R¹² is a hydrogen atom, R¹³ is a hydrogen atom, R¹⁴ is a methyl group, R¹⁵ is a methyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX626).

The compound (L-2), wherein R¹² is a hydrogen atom, R¹³ is a hydrogen atom, R¹⁴ is a methyl group, R¹⁵ is a hydrogen atom, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX627).

The compound (L-2), wherein R¹² is a hydrogen atom, R¹³ is a hydrogen atom, R¹⁴ is a hydrogen atom, R¹⁵ is a methyl group, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX628).

The compound (L-2), wherein R¹² is a hydrogen atom, R¹³ is a methyl group, R¹⁴ is a methyl group, R¹⁵ is a methyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX629).

The compound (L-2), wherein R¹² is a hydrogen atom, R¹³ is a hydrogen atom, R¹⁴ is a methyl group, R¹⁵ is a methyl group, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX630).

The compound (L-2), wherein R¹² is a hydrogen atom, R¹³ is a fluorine atom, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX631).

The compound (L-2), wherein R¹² is a hydrogen atom, R¹³ is a hydrogen atom, R¹⁴ is a fluorine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX632).

The compound (L-2), wherein R¹² is a hydrogen atom, R¹³ is a hydrogen atom, R¹⁴ is a hydrogen atom, R¹⁵ is a fluorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX633).

The compound (L-2), wherein R¹² is a hydrogen atom, R¹³ is a hydrogen atom, R¹⁴ is a hydrogen atom, R¹⁵ is a hydrogen atom, R¹⁶ is a fluorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX634).

The compound (L-2), wherein R¹² is a hydrogen atom, R¹³ is a chlorine atom, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX635).

The compound (L-2), wherein R¹² is a hydrogen atom, R¹³ is a hydrogen atom, R¹⁴ is a chlorine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX636).

The compound (L-2), wherein R¹² is a hydrogen atom, R¹³ is a hydrogen atom, R¹⁴ is a hydrogen atom, R¹⁵ is a chlorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX637).

The compound (L-2), wherein R¹² is a hydrogen atom, R¹³ is a hydrogen atom, R¹⁴ is a hydrogen atom, R¹⁵ is a hydrogen atom, R¹⁶ is a chlorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX638).

The compound (L-2), wherein R¹² is a hydrogen atom, R¹³ is a bromine atom, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX639).

The compound (L-2), wherein R¹² is a hydrogen atom, R¹³ is a hydrogen atom, R¹⁴ is a bromine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX640).

The compound (L-2), wherein R¹² is a hydrogen atom, R¹³ is a hydrogen atom, R¹⁴ is a hydrogen atom, R¹⁵ is a bromine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX641).

The compound (L-2), wherein R¹² is a hydrogen atom, R¹³ is a hydrogen atom, R¹⁴ is a hydrogen atom, R¹⁵ is a hydrogen atom, R¹⁶ is a bromine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX642).

The compound (L-2), wherein R¹² is a hydrogen atom, R¹³ is an iodine atom, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX643).

The compound (L-2), wherein R¹² is a hydrogen atom, R¹³ is a hydrogen atom, R¹⁴ is an iodine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX644).

The compound (L-2), wherein R¹² is a hydrogen atom, R¹³ is a hydrogen atom, R¹⁴ is a hydrogen atom, R¹⁵ is an iodine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table 99] (hereinafter, referred to as a compound group SX645).

The compound (L-2), wherein R¹² is a hydrogen atom, R¹³ is a hydrogen atom, R¹⁴ is a hydrogen atom, R¹⁵ is a hydrogen atom, R¹⁶ is an iodine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX646).

The compound (L-2), wherein R¹² is a hydrogen atom, R¹³ is a methoxy group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX647).

The compound (L-2), wherein R¹² is a hydrogen atom, R¹³ is a hydrogen atom, R¹⁴ is a methoxy group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX648).

The compound (L-2), wherein R¹² is a hydrogen atom, R¹³ is a hydrogen atom, R¹⁴ is a hydrogen atom, R¹⁵ is a methoxy group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX649).

The compound (L-2), wherein R¹² is a hydrogen atom, R¹³ is a hydrogen atom, R¹⁴ is a hydrogen atom, R¹⁵ is a hydrogen atom, R¹⁶ is a methoxy group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX650).

The compound (L-2), wherein R¹² is a hydrogen atom, R¹³ is a trifluoromethyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX651).

The compound (L-2), wherein R¹² is a hydrogen atom, R¹³ is a hydrogen atom, R¹⁴ is a trifluoromethyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX652).

The compound (L-2), wherein R¹² is a hydrogen atom, R¹³ is a hydrogen atom, R¹⁴ is a hydrogen atom, R¹⁵ is a trifluoromethyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX653).

The compound (L-2), wherein R¹² is a hydrogen atom, R¹³ is a hydrogen atom, R¹⁴ is a hydrogen atom, R¹⁵ is a hydrogen atom, R¹⁶ is a trifluoromethyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX654).

The compound (L-2), wherein R¹² is a hydrogen atom, R¹³ is a cyclopropyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX655).

The compound (L-2), wherein R¹² is a hydrogen atom, R¹³ is a hydrogen atom, R¹⁴ is a cyclopropyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX656).

The compound (L-2), wherein R¹² is a hydrogen atom, R¹³ is a hydrogen atom, R¹⁴ is a hydrogen atom, R¹⁵ is a cyclopropyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX657).

The compound (L-2), wherein R¹² is a hydrogen atom, R¹³ is a hydrogen atom, R¹⁴ is a hydrogen atom, R¹⁵ is a hydrogen atom, R¹⁶ is a cyclopropyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX658).

The compound (L-2), wherein R¹² is a hydrogen atom, R¹³ is a cyclobutyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX659).

The compound (L-2), wherein R¹² is a hydrogen atom, R¹³ is a hydrogen atom, R¹⁴ is a cyclobutyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX660).

The compound (L-2), wherein R¹² is a hydrogen atom, R¹³ is a hydrogen atom, R¹⁴ is a hydrogen atom, R¹⁵ is a cyclobutyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX661).

The compound (L-2), wherein R¹² is a hydrogen atom, R¹³ is a hydrogen atom, R¹⁴ is a hydrogen atom, R¹⁵ is a hydrogen atom, R¹⁶ is a cyclobutyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX662).

The compound (L-2), wherein R¹² is a hydrogen atom, R¹³ is a cyclopentyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX663).

The compound (L-2), wherein R¹² is a hydrogen atom, R¹³ is a hydrogen atom, R¹⁴ is a cyclopentyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX664).

The compound (L-2), wherein R¹² is a hydrogen atom, R¹³ is a hydrogen atom, R¹⁴ is a hydrogen atom, R¹⁵ is a cyclopentyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX665).

The compound (L-2), wherein R¹² is a hydrogen atom, R¹³ is a hydrogen atom, R¹⁴ is a hydrogen atom, R¹⁵ is a hydrogen atom, R¹⁶ is a cyclopentyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX666).

The compound (L-2), wherein R¹² is a hydrogen atom, R¹³ is a cyclohexyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX667).

The compound (L-2), wherein R¹² is a hydrogen atom, R¹³ is a hydrogen atom, R¹⁴ is a cyclohexyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX668).

The compound (L-2), wherein R¹² is a hydrogen atom, R¹³ is a hydrogen atom, R¹⁴ is a hydrogen atom, R¹⁵ is a cyclohexyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX669).

The compound (L-2), wherein R¹² is a hydrogen atom, R¹³ is a hydrogen atom, R¹⁴ is a hydrogen atom, R¹⁵ is a hydrogen atom, R¹⁶ is a cyclohexyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX670).

The compound (L-2), wherein R¹² is a hydrogen atom, R¹³ is a phenyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX671).

The compound (L-2), wherein R¹² is a hydrogen atom, R¹³ is a hydrogen atom, R¹⁴ is a phenyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX672).

The compound (L-2), wherein R¹² is a hydrogen atom, R¹³ is a hydrogen atom, R¹⁴ is a hydrogen atom, R¹⁵ is a phenyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX673).

The compound (L-2), wherein R¹² is a hydrogen atom, R¹³ is a hydrogen atom, R¹⁴ is a hydrogen atom, R¹⁵ is a hydrogen atom, R¹⁶ is a phenyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX674).

The compound (L-2), wherein R¹² is a hydrogen atom, R¹³ is an acetyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX675).

The compound (L-2), wherein R¹² is a hydrogen atom, R¹³ is a hydrogen atom, R¹⁴ is an acetyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX676).

The compound (L-2), wherein R¹² is a hydrogen atom, R¹³ is a hydrogen atom, R¹⁴ is a hydrogen atom, R¹⁵ is an acetyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX677).

The compound (L-2), wherein R¹² is a hydrogen atom, R¹³ is a hydrogen atom, R¹⁴ is a hydrogen atom, R¹⁵ is a hydrogen atom, R¹⁶ is an acetyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX678).

The compound (L-2), wherein R¹² is a hydrogen atom, R¹³ is a trifluoroacetyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX679).

The compound (L-2), wherein R¹² is a hydrogen atom, R¹³ is a hydrogen atom, R¹⁴ is a trifluoroacetyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX680).

The compound (L-2), wherein R¹² is a hydrogen atom, R¹³ is a hydrogen atom, R¹⁴ is a hydrogen atom, R¹⁵ is a trifluoroacetyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX681).

The compound (L-2), wherein R¹² is a hydrogen atom, R¹³ is a hydrogen atom, R¹⁴ is a hydrogen atom, R¹⁵ is a hydrogen atom, R¹⁶ is a trifluoroacetyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX682).

The compound (L-2), wherein R¹² is a hydrogen atom, R¹³ is a methoxycarbonyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX683).

The compound (L-2), wherein R¹² is a hydrogen atom, R¹³ is a hydrogen atom, R¹⁴ is a methoxycarbonyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX684).

The compound (L-2), wherein R¹² is a hydrogen atom, R¹³ is a hydrogen atom, R¹⁴ is a hydrogen atom, R¹⁵ is a methoxycarbonyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX685).

The compound (L-2), wherein R¹² is a hydrogen atom, R¹³ is a hydrogen atom, R¹⁴ is a hydrogen atom, R¹⁵ is a hydrogen atom, R¹⁶ is a methoxycarbonyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX686).

The compound (L-2), wherein R¹² is a hydrogen atom, R¹³ is a trifluoromethoxycarbonyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX687).

The compound (L-2), wherein R¹² is a hydrogen atom, R¹³ is a hydrogen atom, R¹⁴ is a trifluoromethoxycarbonyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX688).

The compound (L-2), wherein R¹² is a hydrogen atom, R¹³ is a hydrogen atom, R¹⁴ is a hydrogen atom, R¹⁵ is a trifluoromethoxycarbonyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX689).

The compound (L-2), wherein R¹² is a hydrogen atom, R¹³ is a hydrogen atom, R¹⁴ is a hydrogen atom, R¹⁵ is a hydrogen atom, R¹⁶ is a trifluoromethoxycarbonyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX690).

The compound (L-2), wherein R¹² is a hydrogen atom, R¹³ is an acetoxy group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99 (hereinafter, referred to as a compound group SX691).

The compound (L-2), wherein R¹² is a hydrogen atom, R¹³ is a hydrogen atom, R¹⁴ is an acetoxy group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX692).

The compound (L-2), wherein R¹² is a hydrogen atom, R¹³ is a hydrogen atom, R¹⁴ is a hydrogen atom, R¹⁵ is an acetoxy group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX693).

The compound (L-2), wherein R¹² is a hydrogen atom, R¹³ is a hydrogen atom, R¹⁴ is a hydrogen atom, R¹⁵ is a hydrogen atom, R¹⁶ is an acetoxy group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX694).

The compound (L-2), wherein R¹² is a hydrogen atom, R¹³ is a trifluoroacetoxy group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and Ra is any one of the combinations shown in [Table L13] to [Table L99] (hereinafter, referred to as a compound group SX695).

The compound (L-2), wherein R¹² is a hydrogen atom, R¹³ is a hydrogen atom, R¹⁴ is a trifluoroacetoxy group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX696).

The compound (L-2), wherein R¹² is a hydrogen atom, R¹³ is a hydrogen atom, R¹⁴ is a hydrogen atom, R¹⁵ is a trifluoroacetoxy group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX697).

The compound (L-2), wherein R¹² is a hydrogen atom, R¹³ is a hydrogen atom, R¹⁴ is a hydrogen atom, R¹⁵ is a hydrogen atom, R¹⁶ is a trifluoroacetoxy group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX698).

The compound (L-2), wherein R¹² is a hydrogen atom, R¹³ is a cyano group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX699).

The compound (L-2), wherein R¹² is a hydrogen atom, R¹³ is a hydrogen atom, R¹⁴ is a cyano group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX700).

The compound (L-2), wherein R¹² is a hydrogen atom, R¹³ is a hydrogen atom, R¹⁴ is a hydrogen atom, R¹⁵ is a cyano group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX701).

The compound (L-2), wherein R¹² is a hydrogen atom, R¹³ is a hydrogen atom, R¹⁴ is a hydrogen atom, R¹⁵ is a hydrogen atom, R¹⁶ is a cyano group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX702).

The compound (L-2), wherein R¹² is a hydrogen atom, R¹³ is a formyl group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX703).

The compound (L-2), wherein R¹² is a hydrogen atom, R¹³ is a hydrogen atom, R¹⁴ is a formyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX704).

The compound (L-2), wherein R¹² is a hydrogen atom, R¹³ is a hydrogen atom, R¹⁴ is a hydrogen atom, R¹⁵ is a formyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX705).

The compound (L-2), wherein R¹² is a hydrogen atom, R¹³ is a hydrogen atom, R¹⁴ is a hydrogen atom, R¹⁵ is a hydrogen atom, R¹⁶ is a formyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX706).

The compound (L-2), wherein R¹² is a hydrogen atom, R¹³ is a carboxy group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX707).

The compound (L-2), wherein R¹² is a hydrogen atom, R¹³ is a hydrogen atom, R¹⁴ is a carboxy group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX708).

The compound (L-2), wherein R¹² is a hydrogen atom, R¹³ is a hydrogen atom, R¹⁴ is a hydrogen atom, R¹⁵ is a carboxy group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX709).

The compound (L-2), wherein R¹² is a hydrogen atom, R¹³ is a hydrogen atom, R¹⁴ is a hydrogen atom, R¹⁵ is a hydrogen atom, R¹⁶ is a carboxy group, X is an oxygen atom, and the combination of R¹, R², R³, R⁴, R⁵, R⁶, R⁷, and R⁸ is any one of the combinations shown in [Table L1] to [Table L99] (hereinafter, referred to as a compound group SX710).

The compound (L-2), wherein R¹² is a hydrogen atom, R¹³ is a nitro group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX711).

The compound (L-2), wherein R¹² is a hydrogen atom, R¹³ is a hydrogen atom, R¹⁴ is a nitro group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX712).

The compound (L-2), wherein R¹² is a hydrogen atom, R¹³ is a hydrogen atom, R¹⁴ is a hydrogen atom, R¹⁵ is a nitro group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX713).

The compound (L-2), wherein R¹² is a hydrogen atom, R¹³ is a hydrogen atom, R¹⁴ is a hydrogen atom, R¹⁵ is a hydrogen atom, R¹⁶ is a nitro group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX714).

The compound (L-2), wherein R¹² is a hydrogen atom, R¹³ is a hydroxy group, R¹⁴ is a hydrogen atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX715).

The compound (L-2), wherein R¹² is a hydrogen atom, R¹³ is a hydrogen atom, R¹⁴ is a hydroxy group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX716).

The compound (L-2), wherein R¹² is a hydrogen atom, R¹³ is a hydrogen atom, R¹⁴ is a hydrogen atom, R¹⁵ is a hydroxy group, R¹⁶ is a hydrogen atom, X is an oxygen atom, and the combination of R¹, R², R³, R⁴, R⁵, R⁶, R⁷, and R⁸ is any one of the combinations shown in [Table L1] to [Table L99] (hereinafter, referred to as a compound group SX717).

The compound (L-2), wherein R¹² is a hydrogen atom, R¹³ is a hydrogen atom, R¹⁴ is a hydrogen atom, R¹⁵ is a hydrogen atom, R¹⁶ is a hydroxy group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX718).

The compound (L-2), wherein R¹² is a hydrogen atom, R¹³ is a fluorine atom, R¹⁴ is a fluorine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX719).

The compound (L-2), wherein R¹² is a hydrogen atom, R¹³ is a fluorine atom, R¹⁴ is a hydrogen atom, R¹⁵ is a fluorine atom, R¹⁶ is a hydrogen atom, X is an oxygen atom, and the combination of R¹, R², R³, R⁴, R⁵, R⁶, R⁷, and RB is any one of the combinations shown in [Table L13] to [Table L99] (hereinafter, referred to as a compound group SX720).

The compound (L-2), wherein R¹² is a hydrogen atom, R¹³ is a fluorine atom, R¹⁴ is a hydrogen atom, R¹⁵ is a hydrogen atom, R¹⁶ is a fluorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX721).

The compound (L-2), wherein R¹² is a hydrogen atom, R¹³ is a hydrogen atom, R¹⁴ is a fluorine atom, R¹⁵ is a fluorine atom, R¹⁶ is a hydrogen atom, X is an oxygen atom, and the combination of R¹, R², R³, R¹, R⁵, R⁶, R⁷, and R⁸ is any one of the combinations shown in [Table L13] to [Table L99] (hereinafter, referred to as a compound group SX722).

The compound (L-2), wherein R¹² is a hydrogen atom, R¹³ is a hydrogen atom, R¹⁴ is a fluorine atom, R¹⁵ is a hydrogen atom, R¹⁶ is a fluorine atom, X is an oxygen atom, any one of the combinations shown in [Table L13] to [Table L99] (hereinafter, referred to as a compound group SX723).

The compound (L-2), wherein R¹² is a hydrogen atom, R¹³ is a hydrogen atom, R¹⁴ is a hydrogen atom, R¹⁵ is a fluorine atom, R¹⁶ is a fluorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX724).

The compound (L-2), wherein R¹² is a hydrogen atom, R¹³ is a fluorine atom, R¹⁴ is a fluorine atom, R¹⁵ is a fluorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX725).

The compound (L-2), wherein R¹² is a hydrogen atom, R¹³ is a hydrogen atom, R¹⁴ is a fluorine atom, R¹⁵ is a fluorine atom, R¹⁶ is a fluorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX726).

The compound (L-2), wherein R¹² is a hydrogen atom, R¹³ is a chlorine atom, R¹⁴ is a chlorine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX727).

The compound (L-2), wherein R¹² is a hydrogen atom, R¹³ is a chlorine atom, R¹⁴ is a hydrogen atom, R¹⁵ is a chlorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX728).

The compound (L-2), wherein R¹² is a hydrogen atom, R¹³ is a chlorine atom, R¹⁴ is a hydrogen atom, R¹⁵ is a hydrogen atom, R¹⁶ is a chlorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX729).

The compound (L-2), wherein R¹² is a hydrogen atom, R¹³ is a hydrogen atom, R¹⁴ is a chlorine atom, R¹⁵ is a chlorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX730).

The compound (L-2), wherein R¹² is a hydrogen atom, R¹³ is a hydrogen atom, R¹⁴ is a chlorine atom, R¹⁵ is a hydrogen atom, R¹⁶ is a chlorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX731).

The compound (L-2), wherein R¹² is a hydrogen atom, R¹³ is a hydrogen atom, R¹⁴ is a hydrogen atom, R¹⁵ is a chlorine atom, R¹⁶ is a chlorine atom, X is an oxygen atom, and the combination of R¹, R², R³, R⁴, R⁵, R⁶, R⁷, and R⁸ is any one of the combinations shown in [Table L1] to [Table L99] (hereinafter, referred to as a compound group SX732).

The compound (L-2), wherein R¹² is a hydrogen atom, R¹³ is a chlorine atom, R¹⁴ is a chlorine atom, R¹⁵ is a chlorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX733).

The compound (L-2), wherein R¹² is a hydrogen atom, R¹³ is a hydrogen atom, R¹⁴ is a chlorine atom, R¹⁵ is a chlorine atom, R¹⁶ is a chlorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX734).

The compound (L-2), wherein R¹² is a hydrogen atom, R¹³ is a bromine atom, R¹⁴ is a bromine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX735).

The compound (L-2), wherein R¹² is a hydrogen atom, R¹³ is a bromine atom, R¹⁴ is a hydrogen atom, R¹⁵ is a bromine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX736).

The compound (L-2), wherein R¹² is a hydrogen atom, R¹³ is a bromine atom, R¹⁴ is a hydrogen atom, R¹⁵ is a hydrogen atom, R¹⁶ is a bromine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX737).

The compound (L-2), wherein R¹² is a hydrogen atom, R¹³ is a hydrogen atom, R¹⁴ is a bromine atom, R¹⁵ is a bromine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX738).

The compound (L-2), wherein R¹² is a hydrogen atom, R¹³ is a hydrogen atom, R¹⁴ is a bromine atom, R¹⁵ is a hydrogen atom, R¹⁶ is a bromine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX739).

The compound (L-2), wherein R¹² is a hydrogen atom, R¹³ is a hydrogen atom, R¹⁴ is a hydrogen atom, R¹⁵ is a bromine atom, Re is a bromine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX740).

The compound (L-2), wherein R¹² is a hydrogen atom, R¹³ is a bromine atom, R¹⁴ is a bromine atom, R¹⁵ is a bromine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX741).

The compound (L-2), wherein R¹² is a hydrogen atom, R¹³ is a hydrogen atom, R¹⁴ is a bromine atom, R¹⁵ is a bromine atom, R¹⁶ is a bromine atom, X is an oxygen atom, and the combination of R¹, R², R³, R⁴, R⁵, R⁶, R⁷, and R⁸ is any one of the combinations shown in [Table L1] to [Table L99] (hereinafter, referred to as a compound group SX742).

The compound (L-2), wherein R¹² is a hydrogen atom, R¹³ is an iodine atom, R¹⁴ is an iodine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX743).

The compound (L-2), wherein R¹² is a hydrogen atom, R¹³ is an iodine atom, R¹⁴ is a hydrogen atom, R¹⁵ is an iodine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX744).

The compound (L-2), wherein R¹² is a hydrogen atom, R¹³ is an iodine atom, R¹⁴ is a hydrogen atom, R¹⁵ is a hydrogen atom, R¹⁶ is an iodine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX745).

The compound (L-2), wherein R¹² is a hydrogen atom, R¹³ is a hydrogen atom, R¹⁴ is an iodine atom, R¹⁵ is an iodine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX746).

The compound (L-2), wherein R¹² is a hydrogen atom, R¹³ is a hydrogen atom, R¹⁴ is an iodine atom, R¹⁵ is a hydrogen atom, R¹⁶ is an iodine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX747).

The compound (L-2), wherein R¹² is a hydrogen atom, R¹³ is a hydrogen atom, R¹⁴ is a hydrogen atom, R¹⁵ is an iodine atom, R¹⁶ is an iodine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX748).

The compound (L-2), wherein R¹² is a hydrogen atom, R¹³ is an iodine atom, R¹⁴ is an iodine atom, R¹⁵ is an iodine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX749).

The compound (L-2), wherein R¹² is a hydrogen atom, R¹³ is a hydrogen atom, R¹⁴ is an iodine atom, R¹⁵ is an iodine atom, R¹⁶ is an iodine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX750).

The compound (L-2), wherein R¹² is a hydrogen atom, R¹³ is a fluorine atom, R¹⁴ is a chlorine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX751).

The compound (L-2), wherein R¹² is a hydrogen atom, R¹³ is a fluorine atom, R¹⁴ is a hydrogen atom, R¹⁵ is a chlorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX752).

The compound (L-2), wherein R¹² is a hydrogen atom, R¹³ is a fluorine atom, R¹⁴ is a hydrogen atom, R¹⁵ is a hydrogen atom, R¹⁶ is a chlorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX753).

The compound (L-2), wherein R¹² is a hydrogen atom, R¹³ is a chlorine atom, R¹⁴ is a fluorine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX754).

The compound (L-2), wherein R¹² is a hydrogen atom, R¹³ is a chlorine atom, R¹⁴ is a hydrogen atom, R¹⁵ is a fluorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX755).

The compound (L-2), wherein R¹² is a hydrogen atom, R¹³ is a chlorine atom, R¹⁴ is a hydrogen atom, R¹⁵ is a hydrogen atom, R¹⁶ is a fluorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX756).

The compound (L-2), wherein R¹² is a hydrogen atom, R¹³ is a hydrogen atom, R¹⁴ is a fluorine atom, R¹⁵ is a chlorine atom, R¹⁶ is a hydrogen atom, X is an oxygen atom, and the combination of R¹, R², R³, R⁴, R⁵, R⁶, R⁷, and R⁸ is any one of the combinations shown in [Table L1] to [Table L99] (hereinafter, referred to as a compound group SX757).

The compound (L-2), wherein R¹² is a hydrogen atom, R¹³ is a hydrogen atom, R¹⁴ is a fluorine atom, R¹⁵ is a hydrogen atom, R¹⁶ is a chlorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX758).

The compound (L-2), wherein R¹² is a hydrogen atom, R¹³ is a hydrogen atom, R¹⁴ is a chlorine atom, R¹⁵ is a fluorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX759).

The compound (L-2), wherein R¹² is a hydrogen atom, R¹³ is a hydrogen atom, R¹⁴ is a chlorine atom, R¹⁵ is a hydrogen atom, R¹⁶ is a fluorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX760).

The compound (L-2), wherein R¹² is a hydrogen atom, R¹³ is a hydrogen atom, R¹⁴ is a hydrogen atom, R¹⁵ is a fluorine atom, R¹⁶ is a chlorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX761).

The compound (L-2), wherein R¹² is a hydrogen atom, R¹³ is a hydrogen atom, R¹⁴ is a hydrogen atom, R¹⁵ is a chlorine atom, R¹⁶ is a fluorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX762).

The compound (L-2), wherein R¹² is a hydrogen atom, R¹³ is a fluorine atom, R¹⁴ is a methyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX763).

The compound (L-2), wherein R¹² is a hydrogen atom, R¹³ is a fluorine atom, R¹⁴ is a hydrogen atom, R¹⁵ is a methyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX764).

The compound (L-2), wherein R¹² is a hydrogen atom, R¹³ is a fluorine atom, R¹⁴ is a hydrogen atom, R¹⁵ is a hydrogen atom, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX765).

The compound (L-2), wherein R¹² is a hydrogen atom, R¹³ is a methyl group, R¹⁴ is a fluorine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX766).

The compound (L-2), wherein R¹² is a hydrogen atom, R¹³ is a methyl group, R¹⁴ is a hydrogen atom, R¹⁵ is a fluorine atom, R¹⁶ is a hydrogen atom, X is an oxygen atom, and the combination of R¹, R², R³, R⁴, R⁵, R⁶, R⁷, and R⁸ is any one of the combinations shown in [Table L1] to [Table L99] (hereinafter, referred to as a compound group SX767).

The compound (L-2), wherein R¹² is a hydrogen atom, R¹³ is a methyl group, R¹⁴ is a hydrogen atom, R¹⁵ is a hydrogen atom, R¹⁶ is a fluorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX768).

The compound (L-2), wherein R¹² is a hydrogen atom, R¹³ is a hydrogen atom, R¹⁴ is a fluorine atom, R¹⁵ is a methyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX769).

The compound (L-2), wherein R¹² is a hydrogen atom, R¹³ is a hydrogen atom, R¹⁴ is a fluorine atom, R¹⁵ is a hydrogen atom, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX770).

The compound (L-2), wherein R¹² is a hydrogen atom, R¹³ is a hydrogen atom, R¹⁴ is a methyl group, R¹⁵ is a fluorine atom, R¹⁶ is a hydrogen atom, X is an oxygen atom, and the combination of R¹, R², R³, R⁴, R⁵, R⁶, R⁷, and R⁸ is any one of the combinations shown in [Table L1] to [Table L99] (hereinafter, referred to as a compound group SX771).

The compound (L-2), wherein R¹² is a hydrogen atom, R¹³ is a hydrogen atom, R¹⁴ is a methyl group, R¹⁵ is a hydrogen atom, R¹⁶ is a fluorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX772).

The compound (L-2), wherein R¹² is a hydrogen atom, R¹³ is a hydrogen atom, R¹⁴ is a hydrogen atom, R¹⁵ is a fluorine atom, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX773).

The compound (L-2), wherein R¹² is a hydrogen atom, R¹³ is a hydrogen atom, R¹⁴ is a hydrogen atom, R¹⁵ is a methyl group, R¹⁶ is a fluorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX774).

The compound (L-2), wherein R¹² is a hydrogen atom, R¹³ is a fluorine atom, R¹⁴ is a methoxy group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX775).

The compound (L-2), wherein R¹² is a hydrogen atom, R¹³ is a fluorine atom, R¹⁴ is a hydrogen atom, R¹⁵ is a methoxy group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX776).

The compound (L-2), wherein R¹² is a hydrogen atom, R¹³ is a fluorine atom, R¹⁴ is a hydrogen atom, R¹⁵ is a hydrogen atom, R¹⁶ is a methoxy group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX777).

The compound (L-2), wherein R¹² is a hydrogen atom, R¹³ is a methoxy group, R¹⁴ is a fluorine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX778).

The compound (L-2), wherein R¹² is a hydrogen atom, R¹³ is a methoxy group, R¹⁴ is a hydrogen atom, R¹⁵ is a fluorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX779).

The compound (L-2), wherein R¹² is a hydrogen atom, R¹³ is a methoxy group, R¹⁴ is a hydrogen atom, R¹⁵ is a hydrogen atom, R¹⁶ is a fluorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX780).

The compound (L-2), wherein R¹² is a hydrogen atom, R¹³ is a hydrogen atom, R¹⁴ is a fluorine atom, R¹⁵ is a methoxy group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX781).

The compound (L-2), wherein R¹² is a hydrogen atom, R¹³ is a hydrogen atom, R¹⁴ is a fluorine atom, R¹⁵ is a hydrogen atom, R¹⁶ is a methoxy group, X is an oxygen atom, and the combination of R¹, R², R³, R⁴, R⁵, R⁶, R⁷, and R⁸ is any one of the combinations shown in [Table L1] to [Table L99] (hereinafter, referred to as a compound group SX782).

The compound (L-2), wherein R¹² is a hydrogen atom, R¹³ is a hydrogen atom, R¹⁴ is a methoxy group, R¹⁵ is a fluorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX783).

The compound (L-2), wherein R¹² is a hydrogen atom, R¹³ is a hydrogen atom, R¹⁴ is a methoxy group, R¹⁵ is a hydrogen atom, R¹⁶ is a fluorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX784).

The compound (L-2), wherein R¹² is a hydrogen atom, R¹³ is a hydrogen atom, R¹⁴ is a hydrogen atom, R¹⁵ is a fluorine atom, R¹⁶ is a methoxy group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX785).

The compound (L-2), wherein R¹² is a hydrogen atom, R¹³ is a hydrogen atom, R¹⁴ is a hydrogen atom, R¹⁵ is a methoxy group, R¹⁶ is a fluorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX786).

The compound (L-2), wherein R¹² is a hydrogen atom, R¹³ is a chlorine atom, R¹⁴ is a methyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX787).

The compound (L-2), wherein R¹² is a hydrogen atom, R¹³ is a chlorine atom, R¹⁴ is a hydrogen atom, R¹⁵ is a methyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX788).

The compound (L-2), wherein R¹² is a hydrogen atom, R¹³ is a chlorine atom, R¹⁴ is a hydrogen atom, R¹⁵ is a hydrogen atom, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX789).

The compound (L-2), wherein R¹² is a hydrogen atom, R¹³ is a methyl group, R¹⁴ is a chlorine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX790).

The compound (1-2), wherein R¹² is a hydrogen atom, R¹³ is a methyl group, R¹⁴ is a hydrogen atom, R¹⁵ is a chlorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX791).

The compound (L-2), wherein R¹² is a hydrogen atom, R¹³ is a methyl group, R¹⁴ is a hydrogen atom, R¹⁵ is a hydrogen atom, R¹⁶ is a chlorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX792).

The compound (L-2), wherein R¹² is a hydrogen atom, R¹³ is a hydrogen atom, R¹⁴ is a chlorine atom, R¹⁵ is a methyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX793).

The compound (L-2), wherein R¹² is a hydrogen atom, R¹³ is a hydrogen atom, R¹⁴ is a chlorine atom, R¹⁵ is a hydrogen atom, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX794).

The compound (L-2), wherein R¹² is a hydrogen atom, R¹³ is a hydrogen atom, R¹⁴ is a methyl group, R¹⁵ is a chlorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX795).

The compound (L-2), wherein R¹² is a hydrogen atom, R¹³ is a hydrogen atom, R¹⁴ is a methyl group, R¹⁵ is a hydrogen atom, R¹⁶ is a chlorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX796).

The compound (L-2), wherein R¹² is a hydrogen atom, R¹³ is a hydrogen atom, R¹⁴ is a hydrogen atom, R¹⁵ is a chlorine atom, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX797).

The compound (L-2), wherein R¹² is a hydrogen atom, R¹³ is a hydrogen atom, R¹⁴ is a hydrogen atom, R¹⁵ is a methyl group, R¹⁶ is a chlorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX798).

The compound (L-2), wherein R¹² is a hydrogen atom, R¹³ is a chlorine atom, R¹⁴ is a methoxy group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX799).

The compound (L-2), wherein R¹² is a hydrogen atom, R¹³ is a chlorine atom, R¹⁴ is a hydrogen atom, R¹⁵ is a methoxy group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX800).

The compound (L-2), wherein R¹² is a hydrogen atom, R¹³ is a chlorine atom, R¹⁴ is a hydrogen atom, R¹⁵ is a hydrogen atom, R¹⁶ is a methoxy group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX801).

The compound (L-2), wherein R¹² is a hydrogen atom, R¹³ is a methoxy group, R¹⁴ is a chlorine atom, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX802).

The compound (L-2), wherein R¹² is a hydrogen atom, R¹³ is a methoxy group, R¹⁴ is a hydrogen atom, R¹⁵ is a chlorine atom, R¹⁶ is a hydrogen atom, X is an oxygen atom, and the combination of R¹, R², R³, R⁴, R⁵, R⁶, R⁷, and R⁸ is any one of the combinations shown in [Table L1] to [Table L99] (hereinafter, referred to as a compound group SX803).

The compound (L-2), wherein R¹² is a hydrogen atom, R¹³ is a methoxy group, R¹⁴ is a hydrogen atom, R¹⁵ is a hydrogen atom, R¹⁶ is a chlorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX804).

The compound (L-2), wherein R¹² is a hydrogen atom, R¹³ is a hydrogen atom, R¹⁴ is a chlorine atom, R¹⁵ is a methoxy group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX805).

The compound (L-2), wherein R¹² is a hydrogen atom, R¹³ is a hydrogen atom, R¹⁴ is a chlorine atom, R¹⁵ is a hydrogen atom, R¹⁶ is a methoxy group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX806).

The compound (L-2), wherein R¹² is a hydrogen atom, R¹³ is a hydrogen atom, R¹⁴ is a methoxy group, R¹⁵ is a chlorine atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX807).

The compound (L-2), wherein R¹² is a hydrogen atom, R¹³ is a hydrogen atom, R¹⁴ is a methoxy group, R¹⁵ is a hydrogen atom, R¹⁶ is a chlorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX808).

The compound (L-2), wherein R¹² is a hydrogen atom, R¹³ is a hydrogen atom, R¹⁴ is a hydrogen atom, R¹⁵ is a chlorine atom, R¹⁶ is a methoxy group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX809).

The compound (L-2), wherein R¹² is a hydrogen atom, R¹³ is a hydrogen atom, R¹⁴ is a hydrogen atom, R¹⁵ is a methoxy group, R¹⁶ is a chlorine atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX810).

The compound (L-2), wherein R¹² is a hydrogen atom, R¹³ is a methyl group, R¹⁴ is a methoxy group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX811).

The compound (L-2), wherein R¹² is a hydrogen atom, R¹³ is a methyl group, R¹⁴ is a hydrogen atom, R¹⁵ is a methoxy group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX812).

The compound (L-2), wherein R¹² is a hydrogen atom, R¹³ is a methyl group, R¹⁴ is a hydrogen atom, R¹⁵ is a hydrogen atom, R¹⁶ is a methoxy group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX813).

The compound (L-2), wherein R¹² is a hydrogen atom, R¹³ is a methoxy group, R¹⁴ is a methyl group, R¹⁵ is a hydrogen atom, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX814).

The compound (L-2), wherein R¹² is a hydrogen atom, R¹³ is a methoxy group, R¹⁴ is a hydrogen atom, R¹⁵ is a methyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX815).

The compound (L-2), wherein R¹² is a hydrogen atom, R¹³ is a methoxy group, R¹⁴ is a hydrogen atom, R¹⁵ is a hydrogen atom, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX816).

The compound (L-2), wherein R¹² is a hydrogen atom, R¹³ is a hydrogen atom, R¹⁴ is a methyl group, R¹⁵ is a methoxy group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX817).

The compound (L-2), wherein R¹² is a hydrogen atom, R¹³ is a hydrogen atom, R¹⁴ is a methyl group, R¹⁵ is a hydrogen atom, R¹⁶ is a methoxy group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX818).

The compound (1-2), wherein R¹² is a hydrogen atom, R¹³ is a hydrogen atom, R¹⁴ is a methoxy group, R¹⁵ is a methyl group, R¹⁶ is a hydrogen atom, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX819).

The compound (L-2), wherein R¹² is a hydrogen atom, R¹³ is a hydrogen atom, R¹⁴ is a methoxy group, R¹⁵ is a hydrogen atom, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX820).

The compound (L-2), wherein R¹² is a hydrogen atom, R¹³ is a hydrogen atom, R¹⁴ is a hydrogen atom, R¹⁵ is a methyl group, R¹⁶ is a methoxy group, X is an oxygen atom, and the combination of R¹, R², R³, R⁴, R⁵, R⁶, R⁷, and R⁸ is any one of the combinations shown in [Table L1] to [Table L99] (hereinafter, referred to as a compound group SX821).

The compound (L-2), wherein R¹² is a hydrogen atom, R¹³ is a hydrogen atom, R¹⁴ is a hydrogen atom, R¹⁵ is a methoxy group, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX822).

The compound (L-2), wherein R¹² is a hydrogen atom, R¹³ is a hydrogen atom, R¹⁴ is a hydrogen atom, R¹⁵ is a hydrogen atom, R¹⁶ is a methyl group, X is an oxygen atom, and the combination of R¹, R², R³, R⁴, R⁵, R⁶, R⁷, and R⁸ is any one of the combinations shown in [Table L13] to [Table L99] (hereinafter, referred to as a compound group SX823).

The compound (L-2), wherein R¹² is a methyl group, R¹³ is a hydrogen atom, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX824).

The compound (L-2), wherein R¹² is a hydrogen atom, R¹³ is a methyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX825).

The compound (L-2), wherein R¹² is a hydrogen atom, R¹³ is a hydrogen atom, R¹⁴ is a methyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX826).

The compound (L-2), wherein R¹² is a hydrogen atom, R¹³ is a hydrogen atom, R¹⁴ is a hydrogen atom, R¹⁵ is a methyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX827).

The compound (L-2), wherein R¹² is a hydrogen atom, R¹³ is a hydrogen atom, R¹⁴ is a hydrogen atom, R¹⁵ is a hydrogen atom, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX828).

The compound (L-2), wherein R¹² is a hydrogen atom, R¹³ is a methyl group, R¹⁴ is a methyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX829).

The compound (L-2), wherein R¹² is a hydrogen atom, R¹³ is a methyl group, R¹⁴ is a hydrogen atom, R¹⁵ is a methyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX830).

The compound (L-2), wherein R¹² is a hydrogen atom, R¹³ is a methyl group, R¹⁴ is a hydrogen atom, R¹⁵ is a hydrogen atom, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX831).

The compound (L-2), wherein R¹² is a hydrogen atom, R¹³ is a hydrogen atom, R¹⁴ is a methyl group, R¹⁵ is a methyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX832).

The compound (L-2), wherein R¹² is a hydrogen atom, R¹³ is a hydrogen atom, R¹⁴ is a methyl group, R¹⁵ is a hydrogen atom, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX833).

The compound (L-2), wherein R¹² is a hydrogen atom, R¹³ is a hydrogen atom, R¹⁴ is a hydrogen atom, R¹⁵ is a methyl group, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX834).

The compound (L-2), wherein R¹² is a hydrogen atom, R¹³ is a methyl group, R¹⁴ is a methyl group, R¹⁵ is a methyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX835).

The compound (L-2), wherein R¹² is a hydrogen atom, R¹³ is a hydrogen atom, R¹⁴ is a methyl group, R¹⁵ is a methyl group, Rile is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX836).

The compound (L-2), wherein R¹² is a hydrogen atom, R¹³ is a fluorine atom, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX837).

The compound (L-2), wherein R¹² is a hydrogen atom, R¹³ is a hydrogen atom, R¹⁴ is a fluorine atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX838).

The compound (L-2), wherein R¹² is a hydrogen atom, R¹³ is a hydrogen atom, R¹⁴ is a hydrogen atom, R¹⁵ is a fluorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX839).

The compound (L-2), wherein R¹² is a hydrogen atom, R¹³ is a hydrogen atom, R¹⁴ is a hydrogen atom, R¹⁵ is a hydrogen atom, R¹⁶ is a fluorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX840).

The compound (L-2), wherein R¹² is a hydrogen atom, R¹³ is a chlorine atom, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX841).

The compound (L-2), wherein R¹² is a hydrogen atom, R¹³ is a hydrogen atom, R¹⁴ is a chlorine atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX842).

The compound (L-2), wherein R¹² is a hydrogen atom, R¹³ is a hydrogen atom, R¹⁴ is a hydrogen atom, R¹⁵ is a chlorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX843).

The compound (L-2), wherein R¹² is a hydrogen atom, R¹³ is a hydrogen atom, R¹⁴ is a hydrogen atom, R¹⁵ is a hydrogen atom, R¹⁶ is a chlorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX844).

The compound (L-2), wherein R¹² is a hydrogen atom, R¹³ is a bromine atom, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX845).

The compound (L-2), wherein R¹² is a hydrogen atom, R¹³ is a hydrogen atom, R¹⁴ is a bromine atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX846).

The compound (L-2), wherein R¹² is a hydrogen atom, R¹³ is a hydrogen atom, R¹⁴ is a hydrogen atom, R¹⁵ is a bromine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX847).

The compound (L-2), wherein R¹² is a hydrogen atom, R¹³ is a hydrogen atom, R¹⁴ is a hydrogen atom, R¹⁵ is a hydrogen atom, R¹⁶ is a bromine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX848).

The compound (L-2), wherein R¹² is a hydrogen atom, R¹³ is an iodine atom, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX849).

The compound (L-2), wherein R¹² is a hydrogen atom, R¹³ is a hydrogen atom, R¹⁴ is an iodine atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX850).

The compound (L-2), wherein R¹² is a hydrogen atom, R¹³ is a hydrogen atom, R¹⁴ is a hydrogen atom, R¹⁵ is an iodine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX851).

The compound (L-2), wherein R¹² is a hydrogen atom, R¹³ is a hydrogen atom, R¹⁴ is a hydrogen atom, R¹⁵ is a hydrogen atom, R¹⁶ is an iodine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX852).

The compound (L-2), wherein R¹² is a hydrogen atom, R¹³ is a methoxy group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX853).

The compound (L-2), wherein R¹² is a hydrogen atom, R¹³ is a hydrogen atom, R¹⁴ is a methoxy group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX854).

The compound (L-2), wherein R¹² is a hydrogen atom, R¹³ is a hydrogen atom, R¹⁴ is a hydrogen atom, R¹⁵ is a methoxy group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX855).

The compound (L-2), wherein R¹² is a hydrogen atom, R¹³ is a hydrogen atom, R¹⁴ is a hydrogen atom, R¹⁵ is a hydrogen atom, R¹⁶ is a methoxy group, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX856).

The compound (L-2), wherein R¹² is a hydrogen atom, R¹³ is a trifluoromethyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX857).

The compound (L-2), wherein R¹² is a hydrogen atom, R¹³ is a hydrogen atom, R¹⁴ is a trifluoromethyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX858).

The compound (L-2), wherein R¹² is a hydrogen atom, R¹³ is a hydrogen atom, R¹⁴ is a hydrogen atom, R¹⁵ is a trifluoromethyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX859).

The compound (L-2), wherein R¹² is a hydrogen atom, R¹³ is a hydrogen atom, R¹⁴ is a hydrogen atom, R¹⁵ is a hydrogen atom, R¹⁶ is a trifluoromethyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX860).

The compound (L-2), wherein R¹² is a hydrogen atom, R¹³ is a cyclopropyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX861).

The compound (L-2), wherein R¹² is a hydrogen atom, R¹³ is a hydrogen atom, R¹⁴ is a cyclopropyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX862).

The compound (L-2), wherein R¹² is a hydrogen atom, R¹³ is a hydrogen atom, R¹⁴ is a hydrogen atom, R¹⁵ is a cyclopropyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX863).

The compound (L-2), wherein R¹² is a hydrogen atom, R¹³ is a hydrogen atom, R¹⁴ is a hydrogen atom, R¹⁵ is a hydrogen atom, R¹⁶ is a cyclopropyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX864).

The compound (L-2), wherein R¹² is a hydrogen atom, R¹³ is a cyclobutyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX865).

The compound (L-2), wherein R¹² is a hydrogen atom, R¹³ is a hydrogen atom, R¹⁴ is a cyclobutyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX866).

The compound (L-2), wherein R¹² is a hydrogen atom, R¹³ is a hydrogen atom, R¹⁴ is a hydrogen atom, R¹⁵ is a cyclobutyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX867).

The compound (L-2), wherein R¹² is a hydrogen atom, R¹³ is a hydrogen atom, R¹⁴ is a hydrogen atom, R¹⁵ is a hydrogen atom, R¹⁶ is a cyclobutyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX868).

The compound (L-2), wherein R¹² is a hydrogen atom, R¹³ is a cyclopentyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX869).

The compound (L-2), wherein R¹² is a hydrogen atom, R¹³ is a hydrogen atom, R¹⁴ is a cyclopentyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX870).

The compound (L-2), wherein R¹² is a hydrogen atom, R¹³ is a hydrogen atom, R¹⁴ is a hydrogen atom, R¹⁵ is a cyclopentyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX871).

The compound (L-2), wherein R¹² is a hydrogen atom, R¹³ is a hydrogen atom, R¹⁴ is a hydrogen atom, R¹⁵ is a hydrogen atom, R¹⁶ is a cyclopentyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX872).

The compound (L-2), wherein R¹² is a hydrogen atom, R¹³ is a cyclohexyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX873).

The compound (L-2), wherein R¹² is a hydrogen atom, R¹³ is a hydrogen atom, R¹⁴ is a cyclohexyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX874).

The compound (L-2), wherein R¹² is a hydrogen atom, R¹³ is a hydrogen atom, R¹⁴ is a hydrogen atom, R¹⁵ is a cyclohexyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX875).

The compound (L-2), wherein R¹² is a hydrogen atom, R¹³ is a hydrogen atom, R¹⁴ is a hydrogen atom, R¹⁵ is a hydrogen atom, R¹⁶ is a cyclohexyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX876).

The compound (L-2), wherein R¹² is a hydrogen atom, R¹³ is a phenyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX877).

The compound (L-2), wherein R¹² is a hydrogen atom, R¹³ is a hydrogen atom, R¹⁴ is a phenyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX878).

The compound (L-2), wherein R¹² is a hydrogen atom, R¹³ is a hydrogen atom, R¹⁴ is a hydrogen atom, R¹⁵ is a phenyl group, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX879).

The compound (L-2), wherein R¹² is a hydrogen atom, R¹³ is a hydrogen atom, R¹⁴ is a hydrogen atom, R¹⁵ is a hydrogen atom, R¹⁶ is a phenyl group, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX880).

The compound (L-2), wherein R¹² is a hydrogen atom, R¹³ is an acetyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX881).

The compound (L-2), wherein R¹² is a hydrogen atom, R¹³ is a hydrogen atom, R¹⁴ is an acetyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX882).

The compound (L-2), wherein R¹² is a hydrogen atom, R¹³ is a hydrogen atom, R¹⁴ is a hydrogen atom, R¹⁵ is an acetyl group, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX883).

The compound (L-2), wherein R¹² is a hydrogen atom, R¹³ is a hydrogen atom, R¹⁴ is a hydrogen atom, R¹⁵ is a hydrogen atom, R¹⁶ is an acetyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX884).

The compound (L-2), wherein R¹² is a hydrogen atom, R¹³ is a trifluoroacetyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX885).

The compound (L-2), wherein R¹² is a hydrogen atom, R¹³ is a hydrogen atom, R¹⁴ is a trifluoroacetyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX886).

The compound (L-2), wherein R¹² is a hydrogen atom, R¹³ is a hydrogen atom, R¹⁴ is a hydrogen atom, R¹⁵ is a trifluoroacetyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX887).

The compound (L-2), wherein R¹² is a hydrogen atom, R¹³ is a hydrogen atom, R¹⁴ is a hydrogen atom, R¹⁵ is a hydrogen atom, R¹⁶ is a trifluoroacetyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX888).

The compound (L-2), wherein R¹² is a hydrogen atom, R¹³ is a methoxycarbonyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX889).

The compound (L-2), wherein R¹² is a hydrogen atom, R¹³ is a hydrogen atom, R¹⁴ is a methoxycarbonyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX890).

The compound (L-2), wherein R¹² is a hydrogen atom, R¹³ is a hydrogen atom, R¹⁴ is a hydrogen atom, R¹⁵ is a methoxycarbonyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX891).

The compound (L-2), wherein R¹² is a hydrogen atom, R¹³ is a hydrogen atom, R¹⁴ is a hydrogen atom, R¹⁵ is a hydrogen atom, R¹⁶ is a methoxycarbonyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX892).

The compound (L-2), wherein R¹² is a hydrogen atom, R¹³ is a trifluoromethoxycarbonyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX893).

The compound (L-2), wherein R¹² is a hydrogen atom, R¹³ is a hydrogen atom, R¹⁴ is a trifluoromethoxycarbonyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX894).

The compound (L-2), wherein R¹² is a hydrogen atom, R¹³ is a hydrogen atom, R¹⁴ is a hydrogen atom, R¹⁵ is a trifluoromethoxycarbonyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX895).

The compound (L-2), wherein R¹² is a hydrogen atom, R¹³ is a hydrogen atom, R¹⁴ is a hydrogen atom, R¹⁵ is a hydrogen atom, R¹⁶ is a trifluoromethoxycarbonyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX896).

The compound (L-2), wherein R¹² is a hydrogen atom, R¹³ is an acetoxy group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX897).

The compound (L-2), wherein R¹² is a hydrogen atom, R¹³ is a hydrogen atom, R¹⁴ is an acetoxy group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX898).

The compound (L-2), wherein R¹² is a hydrogen atom, R¹³ is a hydrogen atom, R¹⁴ is a hydrogen atom, R¹⁵ is an acetoxy group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX899).

The compound (L-2), wherein R¹² is a hydrogen atom, R¹³ is a hydrogen atom, R¹⁴ is a hydrogen atom, R¹⁵ is a hydrogen atom, R¹⁶ is an acetoxy group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX900).

The compound (L-2), wherein R¹² is a hydrogen atom, R¹³ is a trifluoroacetoxy group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX901).

The compound (L-2), wherein R¹² is a hydrogen atom, R¹³ is a hydrogen atom, R¹⁴ is a trifluoroacetoxy group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX902).

The compound (L-2), wherein R¹² is a hydrogen atom, R¹³ is a hydrogen atom, R¹⁴ is a hydrogen atom, R¹⁵ is a trifluoroacetoxy group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX903).

The compound (L-2), wherein R¹² is a hydrogen atom, R¹³ is a hydrogen atom, R¹⁴ is a hydrogen atom, R¹⁵ is a hydrogen atom, R¹⁶ is a trifluoroacetoxy group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX904).

The compound (L-2), wherein R¹² is a hydrogen atom, R¹³ is a cyano atom, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX905).

The compound (L-2), wherein R¹² is a hydrogen atom, R¹³ is a hydrogen atom, R¹⁴ is a cyano group, R¹⁵ is a and the combination of R¹, R², R³, R⁴, R⁵, R⁶, R⁷, and R⁸ is any one of the combinations shown in [Table L13] to [Table L99] (hereinafter, referred to as a compound group SX906).

The compound (L-2), wherein R¹² is a hydrogen atom, R¹³ is a hydrogen atom, R¹⁴ is a hydrogen atom, R¹⁵ is a cyano group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX907).

The compound (L-2), wherein R¹² is a hydrogen atom, R¹³ is a hydrogen atom, R¹⁴ is a hydrogen atom, R¹⁵ is a hydrogen atom, R¹⁶ is a cyano group, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX908).

The compound (L-2), wherein R¹² is a hydrogen atom, R¹³ is a formyl group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX909).

The compound (L-2), wherein R¹² is a hydrogen atom, R¹³ is a hydrogen atom, R¹⁴ is a formyl group, R¹⁵ is a any one of the combinations shown in [Table L13] to [Table L99] (hereinafter, referred to as a compound group SX910).

The compound (L-2), wherein R¹² is a hydrogen atom, R¹³ is a hydrogen atom, R¹⁴ is a hydrogen atom, R¹⁵ is a formyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX911).

The compound (L-2), wherein R¹² is a hydrogen atom, R¹³ is a hydrogen atom, R¹⁴ is a hydrogen atom, R¹⁵ is a hydrogen atom, R¹⁶ is a formyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX912).

The compound (L-2), wherein R¹² is a hydrogen atom, R¹³ is a carboxy group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX913).

The compound (L-2), wherein R¹² is a hydrogen atom, R¹³ is a hydrogen atom, R¹⁴ is a carboxy group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX914).

The compound (L-2), wherein R¹² is a hydrogen atom, R¹³ is a hydrogen atom, R¹⁴ is a hydrogen atom, R¹⁵ is a carboxy group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX915).

The compound (L-2), wherein R¹² is a hydrogen atom, R¹³ is a hydrogen atom, R¹⁴ is a hydrogen atom, R¹⁵ is a hydrogen atom, R¹⁶ is a carboxy atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX916).

The compound (L-2), wherein R¹² is a hydrogen atom, R¹³ is a nitro group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX917).

The compound (L-2), wherein R¹² is a hydrogen atom, R¹³ is a hydrogen atom, R¹⁴ is a nitro group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX918).

The compound (L-2), wherein R¹² is a hydrogen atom, R¹³ is a hydrogen atom, R¹⁴ is a hydrogen atom, R¹⁵ is a nitro group, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX919).

The compound (L-2), wherein R¹² is a hydrogen atom, R¹³ is a hydrogen atom, R¹⁴ is a hydrogen atom, R¹⁵ is a hydrogen atom, R¹⁶ is a nitro group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX920).

The compound (L-2), wherein R¹² is a hydrogen atom, R¹³ is a hydroxy group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX921).

The compound (L-2), wherein R¹² is a hydrogen atom, R¹³ is a hydrogen atom, R¹⁴ is a hydroxy group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX922).

The compound (L-2), wherein R¹² is a hydrogen atom, R¹³ is a hydrogen atom, R¹⁴ is a hydrogen atom, R¹⁵ is a hydroxy group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX923).

The compound (L-2), wherein R¹² is a hydrogen atom, R¹³ is a hydrogen atom, R¹⁴ is a hydrogen atom, R¹⁵ is a hydrogen atom, R¹⁶ is a hydroxy group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX924).

The compound (L-2), wherein R¹² is a hydrogen atom, R¹³ is a fluorine atom, R¹⁴ is a fluorine atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX925).

The compound (L-2), wherein R¹² is a hydrogen atom, R¹³ is a fluorine atom, R¹⁴ is a hydrogen atom, R¹⁵ is a fluorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX926).

The compound (L-2), wherein R¹² is a hydrogen atom, R¹³ is a fluorine atom, R¹⁴ is a hydrogen atom, R¹⁵ is a hydrogen atom, R¹⁶ is a fluorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX927).

The compound (1-2), wherein R¹² is a hydrogen atom, R¹³ is a hydrogen atom, R¹⁴ is a fluorine atom, R¹⁵ is a fluorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX928).

The compound (L-2), wherein R¹² is a hydrogen atom, R¹³ is a hydrogen atom, R¹⁴ is a fluorine atom, R¹⁵ is a hydrogen atom, R¹⁶ is a fluorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX929).

The compound (L-2), wherein R¹² is a hydrogen atom, R¹³ is a hydrogen atom, R¹⁴ is a hydrogen atom, R¹⁵ is a fluorine atom, R¹⁶ is a fluorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX930).

The compound (L-2), wherein R¹² is a hydrogen atom, R¹³ is a fluorine atom, R¹⁴ is a fluorine atom, R¹⁵ is a fluorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX931).

The compound (L-2), wherein R¹² is a hydrogen atom, R¹³ is a hydrogen atom, R¹⁴ is a fluorine atom, R¹⁵ is a fluorine atom, R¹⁶ is a fluorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX932).

The compound (L-2), wherein R¹² is a hydrogen atom, R¹³ is a chlorine atom, R¹⁴ is a chlorine atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX933).

The compound (L-2), wherein R¹² is a hydrogen atom, R¹³ is a chlorine atom, R¹⁴ is a hydrogen atom, R¹⁵ is a chlorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX934).

The compound (L-2), wherein R¹² is a hydrogen atom, R¹³ is a chlorine atom, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX935).

The compound (L-2), wherein R¹² is a hydrogen atom, R¹³ is a hydrogen atom, R¹⁴ is a chlorine atom, R¹⁵ is a chlorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX936).

The compound (L-2), wherein R¹² is a hydrogen atom, R¹³ is a hydrogen atom, R¹⁴ is a chlorine atom, R¹⁵ is a hydrogen atom, R¹⁶ is a chlorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX937).

The compound (L-2), wherein R¹² is a hydrogen atom, R¹³ is a hydrogen atom, R¹⁴ is a hydrogen atom, R¹⁵ is a chlorine atom, R¹⁶ is a chlorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX938).

The compound (L-2), wherein R¹² is a hydrogen atom, R¹³ is a chlorine atom, R¹⁴ is a chlorine atom, R¹⁵ is a chlorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX939).

The compound (L-2), wherein R¹² is a hydrogen atom, R¹³ is a hydrogen atom, R¹⁴ is a chlorine atom, R¹⁵ is a chlorine atom, R¹⁶ is a chlorine atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX940).

The compound (L-2), wherein R¹² is a hydrogen atom, R¹³ is a bromine atom, R¹⁴ is a bromine atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX941).

The compound (L-2), wherein R¹² is a hydrogen atom, R¹³ is a bromine atom, R¹⁴ is a hydrogen atom, R¹⁵ is a bromine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX942).

The compound (L-2), wherein R¹² is a hydrogen atom, R¹³ is a bromine atom, R¹⁴ is a hydrogen atom, R¹⁵ is a hydrogen atom, R¹⁶ is a bromine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX943).

The compound (L-2), wherein R¹² is a hydrogen atom, R¹³ is a hydrogen atom, R¹⁴ is a bromine atom, R¹⁵ is a bromine atom, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX944).

The compound (L-2), wherein R¹² is a hydrogen atom, R¹³ is a hydrogen atom, R¹⁴ is a bromine atom, R¹⁵ is a hydrogen atom, R¹⁶ is a bromine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX945).

The compound (L-2), wherein R¹² is a hydrogen atom, R¹³ is a hydrogen atom, R¹⁴ is a hydrogen atom, R¹⁵ is a bromine atom, R¹⁶ is a bromine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX946).

The compound (L-2), wherein R¹² is a hydrogen atom, R¹³ is a bromine atom, R¹⁴ is a bromine atom, R¹⁵ is a bromine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX947).

The compound (L-2), wherein R¹² is a hydrogen atom, R¹³ is a hydrogen atom, R¹⁴ is a bromine atom, R¹⁵ is a bromine atom, R¹⁶ is a bromine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX948).

The compound (L-2), wherein R¹² is a hydrogen atom, R¹³ is an iodine atom, R¹⁴ is an iodine atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX949).

The compound (L-2), wherein R¹² is a hydrogen atom, R¹³ is an iodine atom, R¹⁴ is a hydrogen atom, R¹⁵ is an iodine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX950).

The compound (L-2), wherein R¹² is a hydrogen atom, R¹³ is an iodine atom, R¹⁴ is a hydrogen atom, R¹⁵ is a hydrogen atom, R¹⁶ is an iodine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX951).

The compound (L-2), wherein R¹² is a hydrogen atom, R¹³ is a hydrogen atom, R¹⁴ is an iodine atom, R¹⁵ is an iodine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX952).

The compound (L-2), wherein R¹² is a hydrogen atom, R¹³ is a hydrogen atom, R¹⁴ is an iodine atom, R¹⁵ is a hydrogen atom, R¹⁶ is an iodine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX953).

The compound (L-2), wherein R¹² is a hydrogen atom, R¹³ is a hydrogen atom, R¹⁴ is a hydrogen atom, R¹⁵ is an iodine atom, R¹⁶ is an iodine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX954).

The compound (L-2), wherein R¹² is a hydrogen atom, R¹³ is an iodine atom, R¹⁴ is an iodine atom, R¹⁵ is an iodine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX955).

The compound (L-2), wherein R¹² is a hydrogen atom, R¹³ is a hydrogen atom, R¹⁴ is an iodine atom, R¹⁵ is an iodine atom, R¹⁶ is an iodine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX956).

The compound (L-2), wherein R¹² is a hydrogen atom, R¹³ is a fluorine atom, R¹⁴ is a chlorine atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX957).

The compound (L-2), wherein R¹² is a hydrogen atom, R¹³ is a fluorine atom, R¹⁴ is a hydrogen atom, R¹⁵ is a chlorine atom, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX958).

The compound (L-2), wherein R¹² is a hydrogen atom, R¹³ is a fluorine atom, R¹⁴ is a hydrogen atom, R¹⁵ is a hydrogen atom, R¹⁶ is a chlorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX959).

The compound (L-2), wherein R¹² is a hydrogen atom, R¹³ is a chlorine atom, R¹⁴ is a fluorine atom, R¹⁵ is a any one of the combinations shown in [Table L13] to [Table L99] (hereinafter, referred to as a compound group SX960).

The compound (L-2), wherein R¹² is a hydrogen atom, R¹³ is a chlorine atom, R¹⁴ is a hydrogen atom, R¹⁵ is a fluorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX961).

The compound (L-2), wherein R¹² is a hydrogen atom, R¹³ is a chlorine atom, R¹⁴ is a hydrogen atom, R¹⁵ is a hydrogen atom, R¹⁶ is a fluorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX962).

The compound (L-2), wherein R¹² is a hydrogen atom, R¹³ is a hydrogen atom, R¹⁴ is a fluorine atom, R¹⁵ is a chlorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX963).

The compound (L-2), wherein R¹² is a hydrogen atom, R¹³ is a hydrogen atom, R¹⁴ is a fluorine atom, R¹⁵ is a hydrogen atom, R¹⁶ is a chlorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX964).

The compound (L-2), wherein R¹² is a hydrogen atom, R¹³ is a hydrogen atom, R¹⁴ is a chlorine atom, R¹⁵ is a fluorine atom, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX965).

The compound (L-2), wherein R¹² is a hydrogen atom, R¹³ is a hydrogen atom, R¹⁴ is a chlorine atom, R¹⁵ is a hydrogen atom, R¹⁶ is a fluorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX966).

The compound (L-2), wherein R¹² is a hydrogen atom, R¹³ is a hydrogen atom, R¹⁴ is a hydrogen atom, R¹⁵ is a fluorine atom, R¹⁶ is a chlorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX967).

The compound (L-2), wherein R¹² is a hydrogen atom, R¹³ is a hydrogen atom, R¹⁴ is a hydrogen atom, R¹⁵ is a chlorine atom, R¹⁶ is a fluorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX968).

The compound (L-2), wherein R¹² is a hydrogen atom, R¹³ is a fluorine atom, R¹⁴ is a methyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX969).

The compound (L-2), wherein R¹² is a hydrogen atom, R¹³ is a fluorine atom, R¹⁴ is a hydrogen atom, R¹⁵ is a methyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX970).

The compound (L-2), wherein R¹² is a hydrogen atom, R¹³ is a fluorine atom, R¹⁴ is a hydrogen atom, R¹⁵ is a hydrogen atom, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX971).

The compound (L-2), wherein R¹² is a hydrogen atom, R¹³ is a methyl group, R¹⁴ is a fluorine atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX972).

The compound (L-2), wherein R¹² is a hydrogen atom, R¹³ is a methyl group, R¹⁴ is a hydrogen atom, R¹⁵ is a fluorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX973).

The compound (L-2), wherein R¹² is a hydrogen atom, R¹³ is a methyl group, R¹⁴ is a hydrogen atom, R¹⁵ is a hydrogen atom, R¹⁶ is a fluorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX974).

The compound (L-2), wherein R¹² is a hydrogen atom, R¹³ is a hydrogen atom, R¹⁴ is a fluorine atom, R¹⁵ is a methyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX975).

The compound (L-2), wherein R¹² is a hydrogen atom, R¹³ is a hydrogen atom, R¹⁴ is a fluorine atom, R¹⁵ is a hydrogen atom, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX976).

The compound (L-2), wherein R¹² is a hydrogen atom, R¹³ is a hydrogen atom, R¹⁴ is a methyl group, R¹⁵ is a fluorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX977).

The compound (L-2), wherein R¹² is a hydrogen atom, R¹³ is a hydrogen atom, R¹⁴ is a methyl group, R¹⁵ is a hydrogen atom, R¹⁶ is a fluorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX978).

The compound (L-2), wherein R¹² is a hydrogen atom, R¹³ is a hydrogen atom, R¹⁴ is a hydrogen atom, R¹⁵ is a fluorine atom, R¹⁶ is a methyl group, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX979).

The compound (L-2), wherein R¹² is a hydrogen atom, R¹³ is a hydrogen atom, R¹⁴ is a hydrogen atom, R¹⁵ is a methyl group, R¹⁶ is a fluorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX980).

The compound (L-2), wherein R¹² is a hydrogen atom, R¹³ is a fluorine atom, R¹⁴ is a methoxy atom, R¹⁵ is a hydrogen atom, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX981).

The compound (L-2), wherein R¹² is a hydrogen atom, R¹³ is a fluorine atom, R¹⁴ is a hydrogen atom, R¹⁵ is a methoxy atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX982).

The compound (L-2), wherein R¹² is a hydrogen atom, R¹³ is a fluorine atom, R¹⁴ is a hydrogen atom, R¹⁵ is a hydrogen atom, R¹⁶ is a methoxy atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX983).

The compound (L-2), wherein R¹² is a hydrogen atom, R¹³ is a methoxy group, R¹⁴ is a fluorine atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX984).

The compound (L-2), wherein R¹² is a hydrogen atom, R¹³ is a methoxy group, R¹⁴ is a hydrogen atom, R¹⁵ is a fluorine atom, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX985).

The compound (L-2), wherein R¹² is a hydrogen atom, R¹³ is a methoxy group, R¹⁴ is a hydrogen atom, R¹⁵ is a hydrogen atom, R¹⁶ is a fluorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX986).

The compound (L-2), wherein R¹² is a hydrogen atom, R¹³ is a hydrogen atom, R¹⁴ is a fluorine atom, R¹⁵ is a methoxy group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX987).

The compound (L-2), wherein R¹² is a hydrogen atom, R¹³ is a hydrogen atom, R¹⁴ is a fluorine atom, R¹⁵ is a hydrogen atom, R¹⁶ is a methoxy group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX988).

The compound (L-2), wherein R¹² is a hydrogen atom, R¹³ is a hydrogen atom, R¹⁴ is a methoxy group, R¹⁵ is a fluorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX989).

The compound (L-2), wherein R¹² is a hydrogen atom, R¹³ is a hydrogen atom, R¹⁴ is a methoxy group, R¹⁵ is a hydrogen atom, R¹⁶ is a fluorine atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX990).

The compound (L-2), wherein R¹² is a hydrogen atom, R¹³ is a hydrogen atom, R¹⁴ is a hydrogen atom, R¹⁵ is a fluorine atom, R¹⁶ is a methoxy group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX991).

The compound (L-2), wherein R¹² is a hydrogen atom, R¹³ is a hydrogen atom, R¹⁴ is a hydrogen atom, R¹⁵ is a methoxy group, R¹⁶ is a fluorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX992).

The compound (L-2), wherein R¹² is a hydrogen atom, R¹³ is a chlorine atom, R¹⁴ is a methyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX993).

The compound (L-2), wherein R¹² is a hydrogen atom, R¹³ is a chlorine atom, R¹⁴ is a hydrogen atom, R¹⁵ is a methyl group, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX994).

The compound (L-2), wherein R¹² is a hydrogen atom, R¹³ is a chlorine atom, R¹⁴ is a hydrogen atom, R¹⁵ is a hydrogen atom, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX995).

The compound (L-2), wherein R¹² is a hydrogen atom, R¹³ is a methyl group, R¹⁴ is a chlorine atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX996).

The compound (L-2), wherein R¹² is a hydrogen atom, R¹³ is a methyl group, R¹⁴ is a hydrogen atom, R¹⁵ is a chlorine atom, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX997).

The compound (L-2), wherein R¹² is a hydrogen atom, R¹³ is a methyl group, R¹⁴ is a hydrogen atom, R¹⁵ is a hydrogen atom, R¹⁶ is a chlorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX998).

The compound (L-2), wherein R¹² is a hydrogen atom, R¹³ is a hydrogen atom, R¹⁴ is a chlorine atom, R¹⁵ is a methyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX999).

The compound (L-2), wherein R¹² is a hydrogen atom, R¹³ is a hydrogen atom, R¹⁴ is a chlorine atom, R¹⁵ is a hydrogen atom, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1000).

The compound (L-2), wherein R¹² is a hydrogen atom, R¹³ is a hydrogen atom, R¹⁴ is a methyl group, R¹⁵ is a chlorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1001).

The compound (L-2), wherein R¹² is a hydrogen atom, R¹³ is a hydrogen atom, R¹⁴ is a methyl group, R¹⁵ is a hydrogen atom, R¹⁶ is a chlorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1002).

The compound (L-2), wherein R¹² is a hydrogen atom, R¹³ is a hydrogen atom, R¹⁴ is a hydrogen atom, R¹⁵ is a chlorine atom, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1003).

The compound (L-2), wherein R¹² is a hydrogen atom, R¹³ is a hydrogen atom, R¹⁴ is a hydrogen atom, R¹⁵ is a methyl group, R¹⁶ is a chlorine atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX1004).

The compound (L-2), wherein R¹² is a hydrogen atom, R¹³ is a chlorine atom, R¹⁴ is a methoxy group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1005).

The compound (L-2), wherein R¹² is a hydrogen atom, R¹³ is a chlorine atom, R¹⁴ is a hydrogen atom, R¹⁵ is a methoxy group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1006).

The compound (L-2), wherein R¹² is a hydrogen atom, R¹³ is a chlorine atom, R¹⁴ is a hydrogen atom, R¹⁵ is a hydrogen atom, R¹⁶ is a methoxy group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1007).

The compound (L-2), wherein R¹² is a hydrogen atom, R¹³ is a methoxy group, R¹⁴ is a chlorine atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX1008).

The compound (L-2), wherein R¹² is a hydrogen atom, R¹³ is a methoxy group, R¹⁴ is a hydrogen atom, R¹⁵ is a chlorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1009).

The compound (L-2), wherein R¹² is a hydrogen atom, R¹³ is a methoxy group, R¹⁴ is a hydrogen atom, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1010).

The compound (L-2), wherein R¹² is a hydrogen atom, R¹³ is a hydrogen atom, R¹⁴ is a chlorine atom, R¹⁵ is a methoxy group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1011).

The compound (L-2), wherein R¹² is a hydrogen atom, R¹³ is a hydrogen atom, R¹⁴ is a chlorine atom, R¹⁵ is a hydrogen atom, R¹⁶ is a methoxy group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1012).

The compound (L-2), wherein R¹² is a hydrogen atom, R¹³ is a hydrogen atom, R¹⁴ is a methoxy group, R¹⁵ is a chlorine atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1013).

The compound (L-2), wherein R¹² is a hydrogen atom, R¹³ is a hydrogen atom, R¹⁴ is a methoxy group, R¹⁵ is a hydrogen atom, R¹⁶ is a chlorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1014).

The compound (L-2), wherein R¹² is a hydrogen atom, R¹³ is a hydrogen atom, R¹⁴ is a hydrogen atom, R¹⁵ is a chlorine atom, R¹⁶ is a methoxy group, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX1015).

The compound (L-2), wherein R¹² is a hydrogen atom, R¹³ is a hydrogen atom, R¹⁴ is a hydrogen atom, R¹⁵ is a methoxy group, R¹⁶ is a chlorine atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1016).

The compound (L-2), wherein R¹² is a hydrogen atom, R¹³ is a methyl group, R¹⁴ is a methoxy group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1017).

The compound (L-2), wherein R¹² is a hydrogen atom, R¹³ is a methyl group, R¹⁴ is a hydrogen atom, R¹⁵ is a methoxy group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1018).

The compound (L-2), wherein R¹² is a hydrogen atom, R¹³ is a methyl group, R¹⁴ is a hydrogen atom, R¹⁵ is a hydrogen atom, R¹⁶ is a methoxy group, X is a sulfur atom, and the combination of R¹, R², R³, R⁴, R⁵, R⁶, R⁷, and R⁸ is any one of the combinations shown in [Table L1] to [Table L99] (hereinafter, referred to as a compound group SX1019).

The compound (L-2), wherein R¹² is a hydrogen atom, R¹³ is a methoxy group, R¹⁴ is a methyl group, R¹⁵ is a hydrogen atom, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1020).

The compound (L-2), wherein R¹² is a hydrogen atom, R¹³ is a methoxy group, R¹⁴ is a hydrogen atom, R¹⁵ is a methyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1021).

The compound (L-2), wherein R¹² is a hydrogen atom, R¹³ is a methoxy group, R¹⁴ is a hydrogen atom, R¹⁵ is a hydrogen atom, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1022).

The compound (L-2), wherein R¹² is a hydrogen atom, R¹³ is a hydrogen atom, R¹⁴ is a methyl group, R¹⁵ is a methoxy group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1023).

The compound (L-2), wherein R¹² is a hydrogen atom, R¹³ is a hydrogen atom, R¹⁴ is a methyl group, R¹⁵ is a hydrogen atom, R¹⁶ is a methoxy group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1024).

The compound (L-2), wherein R¹² is a hydrogen atom, R¹³ is a hydrogen atom, R¹⁴ is a methoxy group, R¹⁵ is a methyl group, R¹⁶ is a hydrogen atom, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1025).

The compound (L-2), wherein R¹² is a hydrogen atom, R¹³ is a hydrogen atom, R¹⁴ is a methoxy group, R¹⁵ is a hydrogen atom, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1026).

The compound (L-2), wherein R¹² is a hydrogen atom, R¹³ is a hydrogen atom, R¹⁴ is a hydrogen atom, R¹⁵ is a methyl group, R¹⁶ is a methoxy group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1027).

The compound (L-2), wherein R¹² is a hydrogen atom, R¹³ is a hydrogen atom, R¹⁴ is a hydrogen atom, R¹⁵ is a methoxy group, R¹⁶ is a methyl group, X is a sulfur atom, and the combination of R¹, R², R³, R⁴, R⁵, R⁶, R⁷, and R⁸ is any one of the combinations shown in [Table L13] to [Table L99] (hereinafter, referred to as a compound group SX1028).

The compound (L-2), wherein R¹² is a hydrogen atom, R¹³ is a hydrogen atom, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1029).

The compound (L-2), wherein R¹² is a methyl group, R¹³ is a hydrogen atom, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1030).

The compound (L-2), wherein R¹² is a hydrogen atom, R¹³ is a methyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1031).

The compound (L-2), wherein R¹² is a hydrogen atom, R¹³ is a hydrogen atom, R¹⁴ is a methyl group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1032).

The compound (L-2), wherein R¹² is a hydrogen atom, R¹³ is a hydrogen atom, R¹⁴ is a hydrogen atom, R¹⁵ is a methyl group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1033).

The compound (L-2), wherein R¹² is a hydrogen atom, R¹³ is a hydrogen atom, R¹⁴ is a hydrogen atom, R¹⁵ is a hydrogen atom, R¹⁶ is a methyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1034).

The compound (L-2), wherein R¹² is a hydrogen atom, R¹³ is a methyl group, R¹⁴ is a methyl group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1035).

The compound (L-2), wherein R¹² is a hydrogen atom, R¹³ is a methyl group, R¹⁴ is a hydrogen atom, R¹⁵ is a methyl group, R¹⁶ is a hydrogen atom, X is NH, and the Combination of R¹, R², R³, R⁴, R⁵, R⁶, R⁷, and R⁸ is any one of the combinations shown in [Table L13] to [Table L99] (hereinafter, referred to as a compound group SX1036).

The compound (L-2), wherein R¹² is a hydrogen atom, R¹³ is a methyl group, R¹⁴ is a hydrogen atom, R¹⁵ is a hydrogen atom, R¹⁶ is a methyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1037).

The compound (L-2), wherein R¹² is a hydrogen atom, R¹³ is a hydrogen atom, R¹⁴ is a methyl group, R¹⁵ is a methyl group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1038).

The compound (L-2), wherein R¹² is a hydrogen atom, R¹³ is a hydrogen atom, R¹⁴ is a methyl group, R¹⁵ is a hydrogen atom, R¹⁶ is a methyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1039).

The compound (L-2), wherein R¹² is a hydrogen atom, R¹³ is a hydrogen atom, R¹⁴ is a hydrogen atom, R¹⁵ is a methyl group, R¹⁶ is a methyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1040).

The compound (L-2), wherein R¹² is a hydrogen atom, R¹³ is a methyl group, R¹⁴ is a methyl group, R¹⁵ is a methyl group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1041).

The compound (L-2), wherein R¹² is a hydrogen atom, R¹³ is a hydrogen atom, R¹⁴ is a methyl group, R¹⁵ is a methyl group, R¹⁶ is a methyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1042).

The compound (L-2), wherein R¹² is a hydrogen atom, R¹³ is a fluorine atom, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1043).

The compound (L-2), wherein R¹² is a hydrogen atom, R¹³ is a hydrogen atom, R¹⁴ is a fluorine atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1044).

The compound (L-2), wherein R¹² is a hydrogen atom, R¹³ is a hydrogen atom, R¹⁴ is a hydrogen atom, R¹⁵ is a fluorine atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1045).

The compound (L-2), wherein R¹² is a hydrogen atom, R¹³ is a hydrogen atom, R¹⁴ is a hydrogen atom, R¹⁵ is a hydrogen atom, R¹⁶ is a fluorine atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1046).

The compound (L-2), wherein R¹² is a hydrogen atom, R¹³ is a chlorine atom, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1047).

The compound (L-2), wherein R¹² is a hydrogen atom, R¹³ is a hydrogen atom, R¹⁴ is a chlorine atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1048).

The compound (L-2), wherein R¹² is a hydrogen atom, R¹³ is a hydrogen atom, R¹⁴ is a hydrogen atom, R¹⁵ is a chlorine atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1049).

The compound (L-2), wherein R¹² is a hydrogen atom, R¹³ is a hydrogen atom, R¹⁴ is a hydrogen atom, R¹⁵ is a hydrogen atom, R¹⁶ is a chlorine atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1050).

The compound (L-2), wherein R¹² is a hydrogen atom, R¹³ is a bromine atom, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1051).

The compound (L-2), wherein R¹² is a hydrogen atom, R¹³ is a hydrogen atom, R¹⁴ is a bromine atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1052).

The compound (L-2), wherein R¹² is a hydrogen atom, R¹³ is a hydrogen atom, R¹⁴ is a hydrogen atom, R¹⁵ is a bromine atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1053).

The compound (L-2), wherein R¹² is a hydrogen atom, R¹³ is a hydrogen atom, R¹⁴ is a hydrogen atom, R¹⁵ is a hydrogen atom, R¹⁶ is a bromine atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1054).

The compound (L-2), wherein R¹² is a hydrogen atom, R¹³ is an iodine atom, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1055).

The compound (L-2), wherein R¹² is a hydrogen atom, R¹³ is a hydrogen atom, R¹⁴ is an iodine atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1056).

The compound (L-2), wherein R¹² is a hydrogen atom, R¹³ is a hydrogen atom, R¹⁴ is a hydrogen atom, R¹⁵ is an iodine atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1057).

The compound (L-2), wherein R¹² is a hydrogen atom, R¹³ is a hydrogen atom, R¹⁴ is a hydrogen atom, R¹⁵ is a hydrogen atom, R¹⁶ is an iodine atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1058).

The compound (L-2), wherein R¹² is a hydrogen atom, R¹³ is a methoxy group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1059).

The compound (L-2), wherein R¹² is a hydrogen atom, R¹³ is a hydrogen atom, R¹⁴ is a methoxy group, Ru5 is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1060).

The compound (L-2), wherein R¹² is a hydrogen atom, R¹³ is a hydrogen atom, R¹⁴ is a hydrogen atom, R¹⁵ is a methoxy group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1061).

The compound (L-2), wherein R¹² is a hydrogen atom, R¹³ is a hydrogen atom, R¹⁴ is a hydrogen atom, R¹⁵ is a hydrogen atom, R¹⁶ is a methoxy group, X is NH, and the combination of R¹, R², R³, R⁴, R⁵, R⁶, R⁷, and R⁸ is any one of the combinations shown in [Table L13] to [Table L99] (hereinafter, referred to as a compound group SX1062).

The compound (L-2), wherein R¹² is a hydrogen atom, R¹³ is a trifluoromethyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1063).

The compound (L-2), wherein R¹² is a hydrogen atom, R¹³ is a hydrogen atom, R¹⁴ is a trifluoromethyl group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1064).

The compound (L-2), wherein R¹² is a hydrogen atom, R¹³ is a hydrogen atom, R¹⁴ is a hydrogen atom, R¹⁵ is a trifluoromethyl group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1065).

The compound (L-2), wherein R¹² is a hydrogen atom, R¹³ is a hydrogen atom, R¹⁴ is a hydrogen atom, R¹⁵ is a hydrogen atom, R¹⁶ is a trifluoromethyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1066).

The compound (L-2), wherein R¹² is a hydrogen atom, R¹³ is a cyclopropyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1067).

The compound (L-2), wherein R¹² is a hydrogen atom, R¹³ is a hydrogen atom, R¹⁴ is a cyclopropyl group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1068).

The compound (L-2), wherein R¹² is a hydrogen atom, R¹³ is a hydrogen atom, R¹⁴ is a hydrogen atom, R¹⁵ is a cyclopropyl group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1069).

The compound (L-2), wherein R¹² is a hydrogen atom, R¹³ is a hydrogen atom, R¹⁴ is a hydrogen atom, R¹⁵ is a hydrogen atom, R¹⁶ is a cyclopropyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1070).

The compound (L-2), wherein R¹² is a hydrogen atom, R¹³ is a cyclobutyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1071).

The compound (L-2), wherein R¹² is a hydrogen atom, R¹³ is a hydrogen atom, R¹⁴ is a cyclobutyl group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1072).

The compound (L-2), wherein R¹² is a hydrogen atom, R¹³ is a hydrogen atom, R¹⁴ is a hydrogen atom, R¹⁵ is a cyclobutyl group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1073).

The compound (L-2), wherein R¹² is a hydrogen atom, R¹³ is a hydrogen atom, R¹⁴ is a hydrogen atom, R¹⁵ is a hydrogen atom, R¹⁶ is a cyclobutyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1074).

The compound (L-2), wherein R¹² is a hydrogen atom, R¹³ is a cyclopentyl group, R¹⁴ is a hydrogen atom, R¹⁵ is a hydrogen atom, R¹⁶ is a hydrogen atom, X is NH, and the Combination of R¹, R², R³, R⁴, R⁵, R⁶, R⁷, and R⁸ is any one of the combinations shown in [Table L13] to [Table L99] (hereinafter, referred to as a compound group SX1075).

The compound (L-2), wherein R¹² is a hydrogen atom, R¹³ is a hydrogen atom, R¹⁴ is a cyclopentyl group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1076).

The compound (L-2), wherein R¹² is a hydrogen atom, R¹³ is a hydrogen atom, R¹⁴ is a hydrogen atom, R¹⁵ is a cyclopentyl group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1077).

The compound (L-2), wherein R¹² is a hydrogen atom, R¹³ is a hydrogen atom, R¹⁴ is a hydrogen atom, R¹⁵ is a hydrogen atom, R¹⁶ is a cyclopentyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1078).

The compound (L-2), wherein R¹² is a hydrogen atom, R¹³ is a cyclohexyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1079).

The compound (L-2), wherein R¹² is a hydrogen atom, R¹³ is a hydrogen atom, R¹⁴ is a cyclohexyl group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1080).

The compound (L-2), wherein R¹² is a hydrogen atom, R¹³ is a hydrogen atom, R¹⁴ is a hydrogen atom, R¹⁵ is a cyclohexyl group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1081).

The compound (L-2), wherein R¹² is a hydrogen atom, R¹³ is a hydrogen atom, R¹⁴ is a hydrogen atom, R¹⁵ is a hydrogen atom, R¹⁶ is a cyclohexyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1082).

The compound (L-2), wherein R¹² is a hydrogen atom, R¹³ is a phenyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1083).

The compound (L-2), wherein R¹² is a hydrogen atom, R¹³ is a hydrogen atom, R¹⁴ is a phenyl group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1084).

The compound (L-2), wherein R¹² is a hydrogen atom, R¹³ is a hydrogen atom, R¹⁴ is a hydrogen atom, R¹⁵ is a phenyl group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1085).

The compound (L-2), wherein R¹² is a hydrogen atom, R¹³ is a hydrogen atom, R¹⁴ is a hydrogen atom, R¹⁵ is a hydrogen atom, R¹⁶ is a phenyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1086).

The compound (1-2), wherein R¹² is a hydrogen atom, R¹³ is an acetyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1087).

The compound (L-2), wherein R¹² is a hydrogen atom, R¹³ is a hydrogen atom, R¹⁴ is an acetyl group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1088).

The compound (L-2), wherein R¹² is a hydrogen atom, R¹³ is a hydrogen atom, R¹⁴ is a hydrogen atom, R¹⁵ is an acetyl group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1089).

The compound (L-2), wherein R¹² is a hydrogen atom, R¹³ is a hydrogen atom, R¹⁴ is a hydrogen atom, R¹⁵ is a hydrogen atom, R¹⁶ is an acetyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1090).

The compound (L-2), wherein R¹² is a hydrogen atom, R¹³ is a trifluoroacetyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1091).

The compound (L-2), wherein R¹² is a hydrogen atom, R¹³ is a hydrogen atom, R¹⁴ is a trifluoroacetyl group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1092).

The compound (L-2), wherein R¹² is a hydrogen atom, R¹³ is a hydrogen atom, R¹⁴ is a hydrogen atom, R¹⁵ is a trifluoroacetyl group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1093).

The compound (L-2), wherein R¹² is a hydrogen atom, R¹³ is a hydrogen atom, R¹⁴ is a hydrogen atom, R¹⁵ is a hydrogen atom, R¹⁶ is a trifluoroacetyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1094).

The compound (L-2), wherein R¹² is a hydrogen atom, R¹³ is a methoxycarbonyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1095).

The compound (L-2), wherein R¹² is a hydrogen atom, R¹³ is a hydrogen atom, R¹⁴ is a methoxycarbonyl group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1096).

The compound (L-2), wherein R¹² is a hydrogen atom, R¹³ is a hydrogen atom, R¹⁴ is a hydrogen atom, R¹⁵ is a methoxycarbonyl group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1097).

The compound (L-2), wherein R¹² is a hydrogen atom, R¹³ is a hydrogen atom, R¹⁴ is a hydrogen atom, R¹⁵ is a hydrogen atom, R¹⁶ is a methoxycarbonyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1098).

The compound (L-2), wherein R¹² is a hydrogen atom, R¹³ is a trifluoromethoxycarbonyl group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and Re is any one of the combinations shown in [Table L13] to [Table L99] (hereinafter, referred to as a compound group SX1099).

The compound (L-2), wherein R¹² is a hydrogen atom, R¹³ is a hydrogen atom, R¹⁴ is a trifluoromethoxycarbonyl group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1100).

The compound (L-2), wherein R¹² is a hydrogen atom, R¹³ is a hydrogen atom, R¹⁴ is a hydrogen atom, R¹⁵ is a trifluoromethoxycarbonyl group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1101).

The compound (L-2), wherein R¹² is a hydrogen atom, R¹³ is a hydrogen atom, R¹⁴ is a hydrogen atom, R¹⁵ is a hydrogen atom, R¹⁶ is a trifluoromethoxycarbonyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1102).

The compound (L-2), wherein R¹² is a hydrogen atom, R¹³ is an acetoxy group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1103).

The compound (L-2), wherein R¹² is a hydrogen atom, R¹³ is a hydrogen atom, R¹⁴ is an acetoxy group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1104).

The compound (L-2), wherein R¹² is a hydrogen atom, R¹³ is a hydrogen atom, R¹⁴ is a hydrogen atom, R¹⁵ is an acetoxy group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1105).

The compound (L-2), wherein R¹² is a hydrogen atom, R¹³ is a hydrogen atom, R¹⁴ is a hydrogen atom, R¹⁵ is a hydrogen atom, R¹⁶ is an acetoxy group, X is NH, and the combination of R¹, R², R³, R⁴, R⁵, R⁶, R⁷, and R⁸ is any one of the combinations shown in [Table L13] to [Table L99] (hereinafter, referred to as a compound group SX1106).

The compound (L-2), wherein R¹² is a hydrogen atom, R¹³ is a trifluoroacetoxy group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1107).

The compound (L-2), wherein R¹² is a hydrogen atom, R¹³ is a hydrogen atom, R¹⁴ is a trifluoroacetoxy group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1108).

The compound (L-2), wherein R¹² is a hydrogen atom, R¹³ is a hydrogen atom, R¹⁴ is a hydrogen atom, R¹⁵ is a trifluoroacetoxy group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1109).

The compound (L-2), wherein R¹² is a hydrogen atom, R¹³ is a hydrogen atom, R¹⁴ is a hydrogen atom, R¹⁵ is a hydrogen atom, R¹⁶ is a trifluoroacetoxy group, X is NH, and the combination of R¹, R², R³, R⁴, R⁵, R⁶, R⁷, and R⁸ is any one of the combinations shown in [Table L1] to [Table L99] (hereinafter, referred to as a compound group SX1110).

The compound (L-2), wherein R¹² is a hydrogen atom, R¹³ is a cyano group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1111).

The compound (L-2), wherein R¹² is a hydrogen atom, R¹³ is a hydrogen atom, R¹⁴ is a cyano group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1112).

The compound (L-2), wherein R¹² is a hydrogen atom, R¹³ is a hydrogen atom, R¹⁴ is a hydrogen atom, R¹⁵ is a cyano group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1113).

The compound (L-2), wherein R¹² is a hydrogen atom, R¹³ is a hydrogen atom, R¹⁴ is a hydrogen atom, R¹⁵ is a hydrogen atom, R¹⁶ is a cyano group, X is NH, and the combination of R¹, R², R³, R⁴, R⁵, R⁶, R⁷, and R⁸ is any one of the combinations shown in [Table L13] to [Table L99] (hereinafter, referred to as a compound group SX1114).

The compound (L-2), wherein R¹² is a hydrogen atom, R¹³ is a formyl group, R¹⁴ is a hydrogen atom, R¹⁵ is a hydrogen atom, R¹⁶ is a formyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1115).

The compound (L-2), wherein R¹² is a hydrogen atom, R¹³ is a hydrogen atom, R¹⁴ is a formyl group, R¹⁵ is a hydrogen atom, R¹⁶ is a formyl group, X is NH, and the Combination of R¹, R², R³, R⁴, R⁵, R⁶, R⁷, and R⁸ is any one of the combinations shown in [Table L13] to [Table L99] (hereinafter, referred to as a compound group SX1116).

The compound (L-2), wherein R¹² is a hydrogen atom, R¹³ is a hydrogen atom, R¹⁴ is a hydrogen atom, R¹⁵ is a formyl group, R¹⁶ is a formyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1117).

The compound (L-2), wherein R¹² is a hydrogen atom, R¹³ is a hydrogen atom, R¹⁴ is a hydrogen atom, R¹⁵ is a hydrogen atom, R¹⁶ is a formyl group, X is NH, and the combination of R¹, R², R³, R⁴, R⁵, R⁶, R⁷, and R⁸ is any one of the combinations shown in [Table L13] to [Table L99] (hereinafter, referred to as a compound group SX1118).

The compound (L-2), wherein R¹² is a hydrogen atom, R¹³ is a carboxy group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1119).

The compound (L-2), wherein R¹² is a hydrogen atom, R¹³ is a hydrogen atom, R¹⁴ is a carboxy group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1120).

The compound (L-2), wherein R¹² is a hydrogen atom, R¹³ is a hydrogen atom, R¹⁴ is a hydrogen atom, R¹⁵ is a carboxy group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1121).

The compound (L-2), wherein R¹² is a hydrogen atom, R¹³ is a hydrogen atom, R¹⁴ is a hydrogen atom, R¹⁵ is a hydrogen atom, R¹⁶ is a carboxy group, X is NH, and the combination of R¹, R², R³, R⁴, R⁵, R⁶, R⁷, and R⁸ is any one of the combinations shown in [Table L13] to [Table L99] (hereinafter, referred to as a compound group SX1122).

The compound (L-2), wherein R¹² is a hydrogen atom, R¹³ is a nitro group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1123).

The compound (L-2), wherein R¹² is a hydrogen atom, R¹³ is a hydrogen atom, R¹⁴ is a nitro group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1124).

The compound (L-2), wherein R¹² is a hydrogen atom, R¹³ is a hydrogen atom, R¹⁴ is a hydrogen atom, R¹⁵ is a nitro group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1125).

The compound (L-2), wherein R¹² is a hydrogen atom, R¹³ is a hydrogen atom, R¹⁴ is a hydrogen atom, R¹⁵ is a hydrogen atom, R¹⁶ is a nitro group, X is NH, and the combination of R¹, R², R³, R⁴, R⁵, R⁶, R⁷, and R⁸ is any one of the combinations shown in [Table L13] to [Table L99] (hereinafter, referred to as a compound group SX1126).

The compound (L-2), wherein R¹² is a hydrogen atom, R¹³ is a hydroxy group, R¹⁴ is a hydrogen atom, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1127).

The compound (L-2), wherein R¹² is a hydrogen atom, R¹³ is a hydrogen atom, R¹⁴ is a hydroxy group, R¹⁵ is a hydrogen atom, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1128).

The compound (L-2), wherein R¹² is a hydrogen atom, R¹³ is a hydrogen atom, R¹⁴ is a hydrogen atom, R¹⁵ is a hydroxy group, R¹⁶ is a hydrogen atom, X is NH, and the combination of R¹, R², R³, R⁴, R⁵, R⁶, R⁷, and R⁸ is any one of the combinations shown in [Table L13] to [Table L99] (hereinafter, referred to as a compound group SX1129).

The compound (L-2), wherein R¹² is a hydrogen atom, R¹³ is a hydrogen atom, R¹⁴ is a hydrogen atom, R¹⁵ is a hydrogen atom, R¹⁶ is a hydroxy group, X is NH, and the combination of R¹, R², R³, R⁴, R⁵, R⁶, R⁷, and R⁸ is any one of the combinations shown in [Table L13] to [Table L99] (hereinafter, referred to as a compound group SX1130).

The compound (L-2), wherein R¹² is a hydrogen atom, R¹³ is a hydrogen atom, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1131).

The compound (L-2), wherein R¹² is a methyl group, R¹³ is a hydrogen atom, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1132).

The compound (1-2), wherein R¹² is a hydrogen atom, R¹³ is a methyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1133).

The compound (L-2), wherein R¹² is a hydrogen atom, R¹³ is a hydrogen atom, R¹⁴ is a methyl group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1134).

The compound (L-2), wherein R¹² is a hydrogen atom, R¹³ is a hydrogen atom, R¹⁴ is a hydrogen atom, R¹⁵ is a methyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1135).

The compound (L-2), wherein R¹² is a hydrogen atom, R¹³ is a hydrogen atom, R¹⁴ is a hydrogen atom, R¹⁵ is a hydrogen atom, R¹⁶ is a methyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1136).

The compound (L-2), wherein R¹² is a hydrogen atom, R¹³ is a methyl group, R¹⁴ is a methyl group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1137).

The compound (L-2), wherein R¹² is a hydrogen atom, R¹³ is a methyl group, R¹⁴ is a hydrogen atom, R¹⁵ is a methyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1138).

The compound (L-2), wherein R¹² is a hydrogen atom, R¹³ is a methyl group, R¹⁴ is a hydrogen atom, R¹⁵ is a hydrogen atom, R¹⁶ is a methyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1139).

The compound (L-2), wherein R¹² is a hydrogen atom, R¹³ is a hydrogen atom, R¹⁴ is a methyl group, R¹⁵ is a methyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1140).

The compound (L-2), wherein R¹² is a hydrogen atom, R¹³ is a hydrogen atom, R¹⁴ is a methyl group, R¹⁵ is a hydrogen atom, R¹⁶ is a methyl group, X is NCH₃, and the Combination of R¹, R², R³, R⁴, R⁵, R⁶, R⁷, and R⁸ is any one of the combinations shown in [Table L13] to [Table L99] (hereinafter, referred to as a compound group SX1141).

The compound (L-2), wherein R¹² is a hydrogen atom, R¹³ is a hydrogen atom, R¹⁴ is a hydrogen atom, R¹⁵ is a methyl group, R¹⁶ is a methyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1142).

The compound (L-2), wherein R¹² is a hydrogen atom, R¹³ is a methyl group, R¹⁴ is a methyl group, R¹⁵ is a methyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1143).

The compound (L-2), wherein R¹² is a hydrogen atom, R¹³ is a hydrogen atom, R¹⁴ is a methyl group, R¹⁵ is a methyl group, R¹⁶ is a methyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1144).

The compound (L-2), wherein R¹² is a hydrogen atom, R¹³ is a fluorine atom, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1145).

The compound (L-2), wherein R¹² is a hydrogen atom, R¹³ is a hydrogen atom, R¹⁴ is a fluorine atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1146).

The compound (L-2), wherein R¹² is a hydrogen atom, R¹³ is a hydrogen atom, R¹⁴ is a hydrogen atom, R¹⁵ is a fluorine atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1147).

The compound (L-2), wherein R¹² is a hydrogen atom, R¹³ is a hydrogen atom, R¹⁴ is a hydrogen atom, R¹⁵ is a hydrogen atom, R¹⁶ is a fluorine atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1148).

The compound (L-2), wherein R¹² is a hydrogen atom, R¹³ is a chlorine atom, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1149).

The compound (L-2), wherein R¹² is a hydrogen atom, R¹³ is a hydrogen atom, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1150).

The compound (L-2), wherein R¹² is a hydrogen atom, R¹³ is a hydrogen atom, R¹⁴ is a hydrogen atom, R¹⁵ is a chlorine atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1151).

The compound (L-2), wherein R¹² is a hydrogen atom, R¹³ is a hydrogen atom, R¹⁴ is a hydrogen atom, R¹⁵ is a hydrogen atom, R¹⁶ is a chlorine atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1152).

The compound (L-2), wherein R¹² is a hydrogen atom, R¹³ is a bromine atom, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1153).

The compound (L-2), wherein R¹² is a hydrogen atom, R¹³ is a hydrogen atom, R¹⁴ is a bromine atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1154).

The compound (L-2), wherein R¹² is a hydrogen atom, R¹³ is a hydrogen atom, R¹⁴ is a hydrogen atom, R¹⁵ is a bromine atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1155).

The compound (L-2), wherein R¹² is a hydrogen atom, R¹³ is a hydrogen atom, R¹⁴ is a hydrogen atom, R¹⁵ is a hydrogen atom, R¹⁶ is a bromine atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1156).

The compound (L-2), wherein R¹² is a hydrogen atom, R¹³ is an iodine atom, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1157).

The compound (L-2), wherein R¹² is a hydrogen atom, R¹³ is a hydrogen atom, R¹⁴ is an iodine atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1158).

The compound (L-2), wherein R¹² is a hydrogen atom, R¹³ is a hydrogen atom, R¹⁴ is a hydrogen atom, R¹⁵ is an iodine atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1159).

The compound (L-2), wherein R¹² is a hydrogen atom, R¹³ is a hydrogen atom, R¹⁴ is a hydrogen atom, R¹⁵ is a hydrogen atom, R¹⁶ is an iodine atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1160).

The compound (L-2), wherein R¹² is a hydrogen atom, R¹³ is a methoxy group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1161).

The compound (L-2), wherein R¹² is a hydrogen atom, R¹³ is a hydrogen atom, R¹⁴ is a methoxy group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1162).

The compound (L-2), wherein R¹² is a hydrogen atom, R¹³ is a hydrogen atom, R¹⁴ is a hydrogen atom, R¹⁵ is a methoxy group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1163).

The compound (L-2), wherein R¹² is a hydrogen atom, R¹³ is a hydrogen atom, R¹⁴ is a hydrogen atom, R¹⁵ is a hydrogen atom, R¹⁶ is a methoxy group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1164).

The compound (L-2), wherein R¹² is a hydrogen atom, R¹³ is a trifluoromethyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1165).

The compound (L-2), wherein R¹² is a hydrogen atom, R¹³ is a hydrogen atom, R¹⁴ is a trifluoromethyl group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1166).

The compound (L-2), wherein R¹² is a hydrogen atom, R¹³ is a hydrogen atom, R¹⁴ is a hydrogen atom, R¹⁵ is a trifluoromethyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1167).

The compound (L-2), wherein R¹² is a hydrogen atom, R¹³ is a hydrogen atom, R¹⁴ is a hydrogen atom, R¹⁵ is a hydrogen atom, R¹⁶ is a trifluoromethyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1168).

The compound (L-2), wherein R¹² is a hydrogen atom, R¹³ is a cyclopropyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1169).

The compound (L-2), wherein R¹² is a hydrogen atom, R¹³ is a hydrogen atom, R¹⁴ is a cyclopropyl group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1170).

The compound (L-2), wherein R¹² is a hydrogen atom, R¹³ is a hydrogen atom, R¹⁴ is a hydrogen atom, R¹⁵ is a cyclopropyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1171).

The compound (L-2), wherein R¹² is a hydrogen atom, R¹³ is a hydrogen atom, R¹⁴ is a hydrogen atom, R¹⁵ is a hydrogen atom, R¹⁶ is a cyclopropyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1172).

The compound (L-2), wherein R¹² is a hydrogen atom, R¹³ is a cyclobutyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R³ is any one of the combinations shown in [Table L13] to [Table L99] (hereinafter, referred to as a compound group SX1173).

The compound (L-2), wherein R¹² is a hydrogen atom, R¹³ is a hydrogen atom, R¹⁴ is a cyclobutyl group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1174).

The compound (L-2), wherein R¹² is a hydrogen atom, R¹³ is a hydrogen atom, R¹⁴ is a hydrogen atom, R¹⁵ is a cyclobutyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1175).

The compound (L-2), wherein R¹² is a hydrogen atom, R¹³ is a hydrogen atom, R¹⁴ is a hydrogen atom, R¹⁵ is a hydrogen atom, R¹⁶ is a cyclobutyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1176).

The compound (L-2), wherein R¹² is a hydrogen atom, R¹³ is a cyclopentyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1177).

The compound (L-2), wherein R¹² is a hydrogen atom, R¹³ is a hydrogen atom, R¹⁴ is a cyclopentyl group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1178).

The compound (L-2), wherein R¹² is a hydrogen atom, R¹³ is a hydrogen atom, R¹⁴ is a hydrogen atom, R¹⁵ is a cyclopentyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1179).

The compound (L-2), wherein R¹² is a hydrogen atom, R¹³ is a hydrogen atom, R¹⁴ is a hydrogen atom, R¹⁵ is a hydrogen atom, R¹⁶ is a cyclopentyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1180).

The compound (L-2), wherein R¹² is a hydrogen atom, R¹³ is a cyclohexyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1181).

The compound (L-2), wherein R¹² is a hydrogen atom, R¹³ is a hydrogen atom, R¹⁴ is a cyclohexyl group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1182).

The compound (L-2), wherein R¹² is a hydrogen atom, R¹³ is a hydrogen atom, R¹⁴ is a hydrogen atom, R¹⁵ is a cyclohexyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1183).

The compound (L-2), wherein R¹² is a hydrogen atom, R¹³ is a hydrogen atom, R¹⁴ is a hydrogen atom, R¹⁵ is a hydrogen atom, R¹⁶ is a cyclohexyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1184).

The compound (L-2), wherein R¹² is a hydrogen atom, R¹³ is a phenyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1185).

The compound (L-2), wherein R¹² is a hydrogen atom, R¹³ is a hydrogen atom, R¹⁴ is a phenyl group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1186).

The compound (L-2), wherein R¹² is a hydrogen atom, R¹³ is a hydrogen atom, R¹⁴ is a hydrogen atom, R¹⁵ is a phenyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1187).

The compound (L-2), wherein R¹² is a hydrogen atom, R¹³ is a hydrogen atom, R¹⁴ is a hydrogen atom, R¹⁵ is a hydrogen atom, R¹⁶ is a phenyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1188).

The compound (L-2), wherein R¹² is a hydrogen atom, R¹³ is an acetyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1189).

The compound (L-2), wherein R¹² is a hydrogen atom, R¹³ is a hydrogen atom, R¹⁴ is an acetyl group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1190).

The compound (L-2), wherein R¹² is a hydrogen atom, R¹³ is a hydrogen atom, R¹⁴ is a hydrogen atom, R¹⁵ is an acetyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1191).

The compound (L-2), wherein R¹² is a hydrogen atom, R¹³ is a hydrogen atom, R¹⁴ is a hydrogen atom, R¹⁵ is a hydrogen atom, R¹⁶ is an acetyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1192).

The compound (L-2), wherein R¹² is a hydrogen atom, R¹³ is a trifluoroacetyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1193).

The compound (L-2), wherein R¹² is a hydrogen atom, R¹³ is a hydrogen atom, R¹⁴ is a trifluoroacetyl group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1194).

The compound (L-2), wherein R¹² is a hydrogen atom, R¹³ is a hydrogen atom, R¹⁴ is a hydrogen atom, R¹⁵ is a trifluoroacetyl group, R¹⁶ is a hydrogen atom, X is NCH₃, and the combination of R¹, R², R³, R⁴, R⁵, R⁶, R⁷, and R⁸ is any one of the combinations shown in [Table L1] to [Table L99] (hereinafter, referred to as a compound group SX1195).

The compound (L-2), wherein R¹² is a hydrogen atom, R¹³ is a hydrogen atom, R¹⁴ is a hydrogen atom, R¹⁵ is a hydrogen atom, R¹⁶ is a trifluoroacetyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1196).

The compound (L-2), wherein R¹² is a hydrogen atom, R¹³ is a methoxycarbonyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1197).

The compound (L-2), wherein R¹² is a hydrogen atom, R¹³ is a hydrogen atom, R¹⁴ is a methoxycarbonyl group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1198).

The compound (L-2), wherein R¹² is a hydrogen atom, R¹³ is a hydrogen atom, R¹⁴ is a hydrogen atom, R¹⁵ is a methoxycarbonyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1199).

The compound (L-2), wherein R¹² is a hydrogen atom, R¹³ is a hydrogen atom, R¹⁴ is a hydrogen atom, R¹⁵ is a hydrogen atom, R¹⁶ is a methoxycarbonyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1200).

The compound (L-2), wherein R¹² is a hydrogen atom, R¹³ is a trifluoromethoxycarbonyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1201).

The compound (L-2), wherein R¹² is a hydrogen atom, R¹³ is a hydrogen atom, R¹⁴ is a trifluoromethoxycarbonyl group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1202).

The compound (L-2), wherein R¹² is a hydrogen atom, R¹³ is a hydrogen atom, R¹⁴ is a hydrogen atom, R¹⁵ is a trifluoromethoxycarbonyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1203).

The compound (L-2), wherein R¹² is a hydrogen atom, R¹³ is a hydrogen atom, R¹⁴ is a hydrogen atom, R¹⁵ is a hydrogen atom, R¹⁶ is a trifluoromethoxycarbonyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1204).

The compound (L-2), wherein R¹² is a hydrogen atom, R¹³ is an acetoxy group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1205).

The compound (L-2), wherein R¹² is a hydrogen atom, R¹³ is a hydrogen atom, R¹⁴ is an acetoxy group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1206).

The compound (L-2), wherein R¹² is a hydrogen atom, R¹³ is a hydrogen atom, R¹⁴ is a hydrogen atom, R¹⁵ is an acetoxy group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1207).

The compound (L-2), wherein R¹² is a hydrogen atom, R¹³ is a hydrogen atom, R¹⁴ is a hydrogen atom, R¹⁵ is a hydrogen atom, R¹⁶ is an acetoxy group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1208).

The compound (L-2), wherein R¹² is a hydrogen atom, R¹³ is a trifluoroacetoxy group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1209).

The compound (L-2), wherein R¹² is a hydrogen atom, R¹³ is a hydrogen atom, R¹⁴ is a trifluoroacetoxy group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1210).

The compound (L-2), wherein R¹² is a hydrogen atom, R¹³ is a hydrogen atom, R¹⁴ is a hydrogen atom, R¹⁵ is a trifluoroacetoxy group, R¹⁶ is a hydrogen atom, X is NCH₃, and the combination of R¹, R², R³, R⁴, R⁵, R⁶, R⁷, and R⁸ is any one of the combinations shown in [Table L1] to [Table L99] (hereinafter, referred to as a compound group SX1211).

The compound (L-2), wherein R¹² is a hydrogen atom, R¹³ is a hydrogen atom, R¹⁴ is a hydrogen atom, R¹⁵ is a hydrogen atom, R¹⁶ is a trifluoroacetoxy group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1212).

The compound (L-2), wherein R¹² is a hydrogen atom, R¹³ is a cyano group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1213).

The compound (L-2), wherein R¹² is a hydrogen atom, R¹³ is a hydrogen atom, R¹⁴ is a cyano group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1214).

The compound (L-2), wherein R¹² is a hydrogen atom, R¹³ is a hydrogen atom, R¹⁴ is a hydrogen atom, R¹⁵ is a cyano group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1215).

The compound (L-2), wherein R¹² is a hydrogen atom, R¹³ is a hydrogen atom, R¹⁴ is a hydrogen atom, R¹⁵ is a hydrogen atom, R¹⁶ is a cyano group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1216).

The compound (L-2), wherein R¹² is a hydrogen atom, R¹³ is a formyl group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1217).

The compound (L-2), wherein R¹² is a hydrogen atom, R¹³ is a hydrogen atom, R¹⁴ is a formyl group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1218).

The compound (L-2), wherein R¹² is a hydrogen atom, R¹³ is a hydrogen atom, R¹⁴ is a hydrogen atom, R¹⁵ is a formyl group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1219).

The compound (L-2), wherein R¹² is a hydrogen atom, R¹³ is a hydrogen atom, R¹⁴ is a hydrogen atom, R¹⁵ is a hydrogen atom, R¹⁶ is a formyl group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1220).

The compound (L-2), wherein R¹² is a hydrogen atom, R¹³ is a carboxy group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1221).

The compound (L-2), wherein R¹² is a hydrogen atom, R¹³ is a hydrogen atom, R¹⁴ is a carboxy group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1222).

The compound (L-2), wherein R¹² is a hydrogen atom, R¹³ is a hydrogen atom, R¹⁴ is a hydrogen atom, R¹⁵ is a carboxy group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1223).

The compound (L-2), wherein R¹² is a hydrogen atom, R¹³ is a hydrogen atom, R¹⁴ is a hydrogen atom, R¹⁵ is a hydrogen atom, R¹⁶ is a carboxy group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1224).

The compound (L-2), wherein R¹² is a hydrogen atom, R¹³ is a nitro group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1225).

The compound (L-2), wherein R¹² is a hydrogen atom, R¹³ is a hydrogen atom, R¹⁴ is a nitro group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1226).

The compound (L-2), wherein R¹² is a hydrogen atom, R¹³ is a hydrogen atom, R¹⁴ is a hydrogen atom, R¹⁵ is a nitro group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1227).

The compound (L-2), wherein R¹² is a hydrogen atom, R¹³ is a hydrogen atom, R¹⁴ is a hydrogen atom, R¹⁵ is a hydrogen atom, R¹⁶ is a nitro group, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1228).

The compound (L-2), wherein R¹² is a hydrogen atom, R¹³ is a hydroxy group, R¹⁴ is a hydrogen atom, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1229).

The compound (L-2), wherein R¹² is a hydrogen atom, R¹³ is a hydroxy atom, R¹⁴ is a hydroxy group, R¹⁵ is a hydrogen atom, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1230).

The compound (L-2), wherein R¹² is a hydrogen atom, R¹³ is a hydroxy atom, R¹⁴ is a hydrogen atom, R¹⁵ is a hydroxy group, R¹⁶ is a hydrogen atom, X is NCH₃, and the combination of R¹, R², R³, R⁴, R⁵, R⁶, R⁷, and R⁸ is any one of the combinations shown in [Table L13] to [Table L99] (hereinafter, referred to as a compound group SX1231).

The compound (L-2), wherein R¹² is a hydrogen atom, R¹³ is a hydroxy atom, R¹⁴ is a hydrogen atom, R¹⁵ is a hydrogen atom, R¹⁶ is a hydroxy group, X is NCH₃, and the Combination of R¹, R², R³, R⁴, R⁵, R⁶, R⁷, and R⁸ is any one of the combinations shown in [Table L13] to [Table L99] (hereinafter, referred to as a compound group SX1232).

A compound represented by a formula (L-3) (hereinafter, referred to as a compound (L-3))

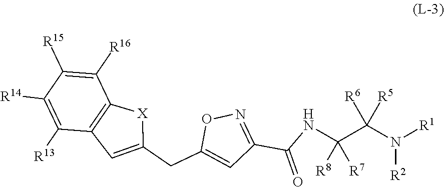

wherein R¹³ is a hydrogen atom, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1233).

TABLE L100

| R¹ | R² | R⁵ | R⁶ | R⁷ | R⁸ |
|---|---|---|---|---|---|
| H | H | H | H | H | H |
| H | H | Me | H | H | H |
| H | H | H | H | Me | H |
| H | H | H | H | H | H |
| H | H | Me | Me | H | H |
| H | H | H | H | Me | Me |
| H | H | H | H | H | H |
| Me | H | H | H | H | H |
| Me | H | Me | H | H | H |
| Me | H | H | H | Me | H |
| Me | H | Me | Me | H | H |
| Me | H | H | H | Me | Me |
| Me | Me | H | H | H | H |
| Me | Me | Me | Me | H | H |
| Me | Me | H | H | Me | Me |
| Me | Me | Me | Me | Me | Me |
| Et | H | H | H | H | H |
| Et | H | Me | H | H | H |
| Et | H | H | H | Me | H |
| Et | H | Me | Me | H | H |
| Et | H | H | H | Me | Me |
| Et | Me | H | H | H | H |
| Et | Me | Me | Me | H | H |
| Et | Me | H | H | Me | Me |
| Et | Me | Me | Me | Me | Me |
| Et | Et | H | H | H | H |
| Et | Et | Me | Me | H | H |
| Et | Et | H | H | Me | Me |
| Et | Et | Me | Me | Me | Me |

TABLE L101

| R¹ | R² | R⁵ | R⁶ | R⁷ | R⁸ |
|---|---|---|---|---|---|
| i-Pr | H | H | H | H | H |
| i-Pr | H | Me | H | H | H |
| i-Pr | H | H | H | Me | H |
| i-Pr | H | Me | Me | H | H |
| i-Pr | H | H | H | Me | Me |
| i-Pr | Me | H | H | H | H |
| i-Pr | Me | Me | Me | H | H |
| i-Pr | Me | H | H | Me | Me |
| i-Pr | Et | H | H | H | H |
| i-Pr | Et | Me | Me | H | H |
| i-Pr | Et | H | H | Me | Me |
| Pr | H | H | H | H | H |
| Pr | H | Me | H | H | H |
| Pr | H | H | H | Me | H |
| Pr | H | Me | Me | H | H |
| Pr | H | H | H | Me | Me |
| Pr | Me | H | H | H | H |
| Pr | Me | Me | Me | H | H |
| Pr | Me | H | H | Me | Me |
| Pr | Et | H | H | H | H |
| Pr | Et | Me | Me | H | H |
| Pr | Et | H | H | Me | Me |
| Pr | i-Pr | H | H | H | H |
| Pr | i-Pr | Me | Me | H | H |
| Pr | i-Pr | H | H | Me | Me |
| Pr | Pr | H | H | H | H |
| Pr | Pr | Me | Me | H | H |
| Pr | Pr | H | H | Me | Me |

TABLE L102

| R¹ | R² | R⁵ | R⁶ | R⁷ | R⁸ |
|---|---|---|---|---|---|
| Bu | H | H | H | H | H |
| Bu | H | Me | H | H | H |
| Bu | H | H | H | Me | H |
| Bu | H | Me | Me | H | H |
| Bu | H | H | H | Me | Me |
| Bu | Me | H | H | H | H |
| Bu | Me | Me | Me | H | H |
| Bu | Me | H | H | Me | Me |
| Bu | Et | H | H | H | H |
| Bu | Et | Me | Me | H | H |
| Bu | Et | H | H | Me | Me |
| Bu | i-Pr | H | H | H | H |
| Bu | i-Pr | Me | Me | H | H |
| Bu | i-Pr | H | H | Me | Me |
| Bu | Pr | H | H | H | H |
| Bu | Pr | Me | Me | H | H |
| Bu | Pr | H | H | Me | Me |
| Bu | Bu | H | H | H | H |
| Bu | Bu | Me | Me | H | H |
| Bu | Bu | H | H | Me | Me |
| t-Bu | H | H | H | H | H |
| t-Bu | H | Me | H | H | H |
| t-Bu | H | H | H | Me | H |
| t-Bu | H | Me | Me | H | H |
| t-Bu | H | H | H | Me | Me |
| t-Bu | Me | H | H | H | H |
| t-Bu | Me | Me | Me | H | H |
| t-Bu | Me | H | H | Me | Me |
| t-Bu | Et | H | H | H | H |
| t-Bu | Et | Me | Me | H | H |
| t-Bu | Et | H | H | Me | Me |
| t-Bu | Pr | H | H | H | H |
| t-Bu | Pr | Me | Me | H | H |
| t-Bu | Pr | H | H | Me | Me |
| t-Bu | Bu | H | H | H | H |
| t-Bu | Bu | Me | Me | H | H |
| t-Bu | Bu | H | H | Me | Me |

TABLE L103

| R¹ | R² | R⁵ | R⁶ | R⁷ | R⁸ |
|---|---|---|---|---|---|
| H | H | Et | Et | H | H |
| H | H | H | H | Et | Et |
| Me | H | Et | Et | H | H |
| Me | H | H | H | Et | Et |
| Me | Me | Et | Et | H | H |
| Me | Me | H | H | Et | Et |
| Et | H | Et | Et | H | H |
| Et | H | H | H | Et | Et |
| Et | Me | Et | Et | H | H |
| Et | Me | H | H | Et | Et |
| Et | Et | Et | Et | H | H |
| Et | Et | H | H | Et | Et |
| i-Pr | H | Et | Et | H | H |
| i-Pr | H | H | H | Et | Et |
| i-Pr | Me | Et | Et | H | H |
| i-Pr | Me | H | H | Et | Et |
| i-Pr | Et | Et | Et | H | H |
| i-Pr | Et | H | H | Et | Et |
| Pr | H | Et | Et | H | H |
| Pr | H | H | H | Et | Et |
| Pr | Me | Et | Et | H | H |
| Pr | Me | H | H | Et | Et |
| Pr | Et | Et | Et | H | H |
| Pr | Et | H | H | Et | Et |
| Pr | i-Pr | Et | Et | H | H |
| Pr | i-Pr | H | H | Et | Et |
| Pr | Pr | Et | Et | H | H |
| Pr | Pr | H | H | Et | Et |

TABLE L104

| R¹ | R² | R⁵ | R⁶ | R⁷ | R⁸ |
|---|---|---|---|---|---|
| Bu | H | Et | Et | H | H |
| Bu | H | H | H | Et | Et |
| Bu | Me | Et | Et | H | H |
| Bu | Me | H | H | Et | Et |
| Bu | Et | Et | Et | H | H |
| Bu | Et | H | H | Et | Et |
| Bu | i-Pr | Et | Et | H | H |
| Bu | i-Pr | H | H | Et | Et |
| Bu | Pr | Et | Et | H | H |
| Bu | Pr | H | H | Et | Et |
| Bu | Bu | Et | Et | H | H |
| Bu | Bu | H | H | Et | Et |
| t-Bu | H | Et | Et | H | H |
| t-Bu | H | H | H | Et | Et |
| t-Bu | Me | Et | Et | H | H |
| t-Bu | Me | H | H | Et | Et |
| t-Bu | Et | Et | Et | H | H |
| t-Bu | Et | H | H | Et | Et |
| t-Bu | Pr | Et | Et | H | H |
| t-Bu | Pr | H | H | Et | Et |
| t-Bu | Bu | Et | Et | H | H |
| t-Bu | Bu | H | H | Et | Et |

TABLE L105

| R¹ | R² | R⁵ | R⁶ | R⁷ | R⁸ |
|---|---|---|---|---|---|
| H | H | i-Pr | H | H | H |
| H | H | H | H | i-Pr | H |
| Me | H | i-Pr | H | H | H |
| Me | H | H | H | i-Pr | H |
| Me | Me | i-Pr | H | H | H |
| Me | Me | H | H | i-Pr | H |
| Et | H | i-Pr | H | H | H |
| Et | H | H | H | i-Pr | H |
| Et | Me | i-Pr | H | H | H |
| Et | Me | H | H | i-Pr | H |
| Et | Et | i-Pr | H | H | H |
| Et | Et | H | H | i-Pr | H |
| i-Pr | H | i-Pr | H | H | H |
| i-Pr | H | H | H | i-Pr | H |
| i-Pr | Me | i-Pr | H | H | H |
| i-Pr | Me | H | H | i-Pr | H |
| i-Pr | Et | i-Pr | H | H | H |
| i-Pr | Et | H | H | i-Pr | H |
| Pr | H | i-Pr | H | H | H |
| Pr | H | H | H | i-Pr | H |
| Pr | Me | i-Pr | H | H | H |
| Pr | Me | H | H | i-Pr | H |
| Pr | Et | i-Pr | H | H | H |
| Pr | Et | H | H | i-Pr | H |
| Pr | i-Pr | i-Pr | H | H | H |
| Pr | i-Pr | H | H | i-Pr | H |
| Pr | Pr | i-Pr | H | H | H |
| Pr | Pr | H | H | i-Pr | H |

TABLE L106

| R¹ | R² | R⁵ | R⁶ | R⁷ | R⁸ |
|---|---|---|---|---|---|
| Bu | H | i-Pr | H | H | H |
| Bu | H | H | H | i-Pr | H |
| Bu | Me | i-Pr | H | H | H |
| Bu | Me | H | H | i-Pr | H |
| Bu | Et | i-Pr | H | H | H |
| Bu | Et | H | H | i-Pr | H |
| Bu | i-Pr | i-Pr | H | H | H |
| Bu | i-Pr | H | H | i-Pr | H |
| Bu | Pr | i-Pr | H | H | H |
| Bu | Pr | H | H | i-Pr | H |
| Bu | Bu | i-Pr | H | H | H |
| Bu | Bu | H | H | i-Pr | H |
| t-Bu | H | i-Pr | H | H | H |
| t-Bu | H | H | H | i-Pr | H |
| t-Bu | Me | i-Pr | H | H | H |
| t-Bu | Me | H | H | i-Pr | H |
| t-Bu | Et | i-Pr | H | H | H |
| t-Bu | Et | H | H | i-Pr | H |
| t-Bu | Pr | i-Pr | H | H | H |
| t-Bu | Pr | H | H | i-Pr | H |
| t-Bu | Bu | i-Pr | H | H | H |
| t-Bu | Bu | H | H | i-Pr | H |

TABLE L107

| R¹ | R² | R⁵ | R⁶ | R⁷ | R⁸ |
|---|---|---|---|---|---|
| H | H | Pr | Pr | H | H |
| H | H | H | H | Pr | Pr |
| Me | H | Pr | Pr | H | H |
| Me | H | H | H | Pr | Pr |
| Me | Me | Pr | Pr | H | H |
| Me | Me | H | H | Pr | Pr |
| Et | H | Pr | Pr | H | H |
| Et | H | H | H | Pr | Pr |
| Et | Me | Pr | Pr | H | H |
| Et | Me | H | H | Pr | Pr |
| Et | Et | Pr | Pr | H | H |
| Et | Et | H | H | Pr | Pr |
| i-Pr | H | Pr | Pr | H | H |
| i-Pr | H | H | H | Pr | Pr |
| i-Pr | Me | Pr | Pr | H | H |
| i-Pr | Me | H | H | Pr | Pr |
| i-Pr | Et | Pr | Pr | H | H |
| i-Pr | Et | H | H | Pr | Pr |
| Pr | H | Pr | Pr | H | H |
| Pr | H | H | H | Pr | Pr |
| Pr | Me | Pr | Pr | H | H |
| Pr | Me | H | H | Pr | Pr |
| Pr | Et | Pr | Pr | H | H |
| Pr | Et | H | H | Pr | Pr |
| Pr | i-Pr | Pr | Pr | H | H |
| Pr | i-Pr | H | H | Pr | Pr |
| Pr | Pr | Pr | Pr | H | H |
| Pr | Pr | H | H | Pr | Pr |

TABLE L108

| R¹ | R² | R⁵ | R⁶ | R⁷ | R⁸ |
|---|---|---|---|---|---|
| Bu | H | Pr | Pr | H | H |
| Bu | H | H | H | Pr | Pr |
| Bu | Me | Pr | Pr | H | H |
| Bu | Me | H | H | Pr | Pr |
| Bu | Et | Pr | Pr | H | H |
| Bu | Et | H | H | Pr | Pr |
| Bu | i-Pr | Pr | Pr | H | H |
| Bu | i-Pr | H | H | Pr | Pr |
| Bu | Pr | Pr | Pr | H | H |
| Bu | Pr | H | H | Pr | Pr |
| Bu | Bu | Pr | Pr | H | H |
| Bu | Bu | H | H | Pr | Pr |
| t-Bu | H | Pr | Pr | H | H |
| t-Bu | H | H | H | Pr | Pr |
| t-Bu | Me | Pr | Pr | H | H |
| t-Bu | Me | H | H | Pr | Pr |
| t-Bu | Et | Pr | Pr | H | H |
| t-Bu | Et | H | H | Pr | Pr |
| t-Bu | Pr | Pr | Pr | H | H |
| t-Bu | Pr | H | H | Pr | Pr |
| t-Bu | Bu | Pr | Pr | H | H |
| t-Bu | Bu | H | H | Pr | Pr |

TABLE L109

| R¹ | R² | R⁵ | R⁶ | R⁷ | R⁸ |
|---|---|---|---|---|---|
| H | H | Bu | Bu | H | H |
| H | H | H | H | Bu | Bu |
| Me | H | Bu | Bu | H | H |
| Me | H | H | H | Bu | Bu |
| Me | Me | Bu | Bu | H | H |
| Me | Me | H | H | Bu | Bu |
| Et | H | Bu | Bu | H | H |
| Et | H | H | H | Bu | Bu |
| Et | Me | Bu | Bu | H | H |
| Et | Me | H | H | Bu | Bu |
| Et | Et | Bu | Bu | H | H |
| Et | Et | H | H | Bu | Bu |
| i-Pr | H | Bu | Bu | H | H |
| i-Pr | H | H | H | Bu | Bu |
| i-Pr | Me | Bu | Bu | H | H |
| i-Pr | Me | H | H | Bu | Bu |
| i-Pr | Et | Bu | Bu | H | H |
| i-Pr | Et | H | H | Bu | Bu |
| Pr | H | Bu | Bu | H | H |
| Pr | H | H | H | Bu | Bu |
| Pr | Me | Bu | Bu | H | H |
| Pr | Me | H | H | Bu | Bu |
| Pr | Et | Bu | Bu | H | H |
| Pr | Et | H | H | Bu | Bu |
| Pr | i-Pr | Bu | Bu | H | H |
| Pr | i-Pr | H | H | Bu | Bu |
| Pr | Pr | Bu | Bu | H | H |
| Pr | Pr | H | H | Bu | Bu |

TABLE L110

| R¹ | R² | R⁵ | R⁶ | R⁷ | R⁸ |
|---|---|---|---|---|---|
| Bu | H | Bu | Bu | H | H |
| Bu | H | H | H | Bu | Bu |
| Bu | Me | Bu | Bu | H | H |
| Bu | Me | H | H | Bu | Bu |
| Bu | Et | Bu | Bu | H | H |
| Bu | Et | H | H | Bu | Bu |
| Bu | i-Pr | Bu | Bu | H | H |
| Bu | i-Pr | H | H | Bu | Bu |
| Bu | Pr | Bu | Bu | H | H |
| Bu | Pr | H | H | Bu | Bu |
| Bu | Bu | Bu | Bu | H | H |
| Bu | Bu | H | H | Bu | Bu |
| t-Bu | H | Bu | Bu | H | H |
| t-Bu | H | H | H | Bu | Bu |
| t-Bu | Me | Bu | Bu | H | H |
| t-Bu | Me | H | H | Bu | Bu |
| t-Bu | Et | Bu | Bu | H | H |
| t-Bu | Et | H | H | Bu | Bu |
| t-Bu | Pr | Bu | Bu | H | H |
| t-Bu | Pr | H | H | Bu | Bu |
| t-Bu | Bu | Bu | Bu | H | H |
| t-Bu | Bu | H | H | Bu | Bu |

TABLE L111

| | | | | | |
|---|---|---|---|---|---|
| R¹ | R² | R⁵ | R⁶ | R⁷ | R⁸ |

| | | | | |
|---|---|---|---|---|
| H | H | —(CH₂)₂— | H | H |

| | | | | |
|---|---|---|---|---|
| H | H | H | H | —(CH₂)₂— |

| | | | | |
|---|---|---|---|---|
| Me | H | —(CH₂)₂— | H | H |

| | | | | |
|---|---|---|---|---|
| Me | H | H | H | —(CH₂)₂— |

| | | | | |
|---|---|---|---|---|
| Me | Me | —(CH₂)₂— | H | H |

| | | | | |
|---|---|---|---|---|
| Me | Me | H | H | —(CH₂)₂— |

| | | | | |
|---|---|---|---|---|
| Et | H | —(CH₂)₂— | H | H |

| | | | | |
|---|---|---|---|---|
| Et | H | H | H | —(CH₂)₂— |

| | | | | |
|---|---|---|---|---|
| Et | Me | —(CH₂)₂— | H | H |

| | | | | |
|---|---|---|---|---|
| Et | Me | H | H | —(CH₂)₂— |

| | | | | |
|---|---|---|---|---|
| Et | Et | —(CH₂)₂— | H | H |

| | | | | |
|---|---|---|---|---|
| Et | Et | H | H | —(CH₂)₂— |

| | | | | |
|---|---|---|---|---|
| i-Pr | H | —(CH₂)₂— | H | H |

| | | | | |
|---|---|---|---|---|
| i-Pr | H | H | H | —(CH₂)₂— |

| | | | | |
|---|---|---|---|---|
| i-Pr | Me | —(CH₂)₂— | H | H |

| | | | | |
|---|---|---|---|---|
| i-Pr | Me | H | H | —(CH₂)₂— |

| | | | | |
|---|---|---|---|---|
| i-Pr | Et | —(CH₂)₂— | H | H |

| | | | | |
|---|---|---|---|---|
| i-Pr | Et | H | H | —(CH₂)₂— |

| | | | | |
|---|---|---|---|---|
| Pr | H | —(CH₂)₂— | H | H |

| | | | | |
|---|---|---|---|---|
| Pr | H | H | H | —(CH₂)₂— |

| | | | | |
|---|---|---|---|---|
| Pr | Me | —(CH₂)₂— | H | H |

| | | | | |
|---|---|---|---|---|
| Pr | Me | H | H | —(CH₂)₂— |

| | | | | |
|---|---|---|---|---|
| Pr | Et | —(CH₂)₂— | H | H |

| | | | | |
|---|---|---|---|---|
| Pr | Et | H | H | —(CH₂)₂— |

| | | | | |
|---|---|---|---|---|
| Pr | i-Pr | —(CH₂)₂— | H | H |

| | | | | |
|---|---|---|---|---|
| Pr | i-Pr | H | H | —(CH₂)₂— |

| | | | | |
|---|---|---|---|---|
| Pr | Pr | —(CH₂)₂— | H | H |

| | | | | |
|---|---|---|---|---|
| Pr | Pr | H | H | —(CH₂)₂— |

TABLE L112

| | | | | | |
|---|---|---|---|---|---|
| R¹ | R² | R⁵ | R⁶ | R⁷ | R⁸ |

| | | | | |
|---|---|---|---|---|
| Bu | H | —(CH₂)₂— | H | H |

| | | | | |
|---|---|---|---|---|
| Bu | H | H | H | —(CH₂)₂— |

| | | | | |
|---|---|---|---|---|
| Bu | Me | —(CH₂)₂— | H | H |

| | | | | |
|---|---|---|---|---|
| Bu | Me | H | H | —(CH₂)₂— |

| | | | | |
|---|---|---|---|---|
| Bu | Et | —(CH₂)₂— | H | H |

| | | | | |
|---|---|---|---|---|
| Bu | Et | H | H | —(CH₂)₂— |

| | | | | |
|---|---|---|---|---|
| Bu | i-Pr | —(CH₂)₂— | H | H |

| | | | | |
|---|---|---|---|---|
| Bu | i-Pr | H | H | —(CH₂)₂— |

| | | | | |
|---|---|---|---|---|
| Bu | Pr | —(CH₂)₂— | H | H |

| | | | | |
|---|---|---|---|---|
| Bu | Pr | H | H | —(CH₂)₂— |

| | | | | |
|---|---|---|---|---|
| Bu | Bu | —(CH₂)₂— | H | H |

| | | | | |
|---|---|---|---|---|
| Bu | Bu | H | H | —(CH₂)₂— |

| | | | | |
|---|---|---|---|---|
| t-Bu | H | —(CH₂)₂— | H | H |

| | | | | |
|---|---|---|---|---|
| t-Bu | H | H | H | —(CH₂)₂— |

| | | | | |
|---|---|---|---|---|
| t-Bu | Me | —(CH₂)₂— | H | H |

| | | | | |
|---|---|---|---|---|
| t-Bu | Me | H | H | —(CH₂)₂— |

| | | | | |
|---|---|---|---|---|
| t-Bu | Et | —(CH₂)₂— | H | H |

| | | | | |
|---|---|---|---|---|
| t-Bu | Et | H | H | —(CH₂)₂— |

| | | | | |
|---|---|---|---|---|
| t-Bu | Pr | —(CH₂)₂— | H | H |

| | | | | |
|---|---|---|---|---|
| t-Bu | Pr | H | H | —(CH₂)₂— |

| | | | | |
|---|---|---|---|---|
| t-Bu | Bu | —(CH₂)₂— | H | H |

| | | | | |
|---|---|---|---|---|
| t-Bu | Bu | H | H | —(CH₂)₂— |

TABLE L113

| | | | | | |
|---|---|---|---|---|---|
| R¹ | R² | R⁵ | R⁶ | R⁷ | R⁸ |

| | | | | |
|---|---|---|---|---|
| H | H | —(CH₂)₃— | H | H |

| | | | | |
|---|---|---|---|---|
| H | H | H | H | —(CH₂)₃— |

| | | | | |
|---|---|---|---|---|
| Me | H | —(CH₂)₃— | H | H |

| | | | | |
|---|---|---|---|---|
| Me | H | H | H | —(CH₂)₃— |

| | | | | |
|---|---|---|---|---|
| Me | Me | —(CH₂)₃— | H | H |

| | | | | |
|---|---|---|---|---|
| Me | Me | H | H | —(CH₂)₃— |

| | | | | |
|---|---|---|---|---|
| Et | H | —(CH₂)₃— | H | H |

| | | | | |
|---|---|---|---|---|
| Et | H | H | H | —(CH₂)₃— |

| | | | | |
|---|---|---|---|---|
| Et | Me | —(CH₂)₃— | H | H |

| | | | | |
|---|---|---|---|---|
| Et | Me | H | H | —(CH₂)₃— |

| | | | | |
|---|---|---|---|---|
| Et | Et | —(CH₂)₃— | H | H |

| | | | | |
|---|---|---|---|---|
| Et | Et | H | H | —(CH₂)₃— |

| | | | | |
|---|---|---|---|---|
| i-Pr | H | —(CH₂)₃— | H | H |

| | | | | |
|---|---|---|---|---|
| i-Pr | H | H | H | —(CH₂)₃— |

| | | | | |
|---|---|---|---|---|
| i-Pr | Me | —(CH₂)₃— | H | H |

| | | | | |
|---|---|---|---|---|
| i-Pr | Me | H | H | —(CH₂)₃— |

| | | | | |
|---|---|---|---|---|
| i-Pr | Et | —(CH₂)₃— | H | H |

| | | | | |
|---|---|---|---|---|
| i-Pr | Et | H | H | —(CH₂)₃— |

| | | | | |
|---|---|---|---|---|
| Pr | H | —(CH₂)₃— | H | H |

| | | | | |
|---|---|---|---|---|
| Pr | H | H | H | —(CH₂)₃— |

| | | | | |
|---|---|---|---|---|
| Pr | Me | —(CH₂)₃— | H | H |

| | | | | |
|---|---|---|---|---|
| Pr | Me | H | H | —(CH₂)₃— |

| | | | | |
|---|---|---|---|---|
| Pr | Et | —(CH₂)₃— | H | H |

| | | | | |
|---|---|---|---|---|
| Pr | Et | H | H | —(CH₂)₃— |

| | | | | |
|---|---|---|---|---|
| Pr | i-Pr | —(CH₂)₃— | H | H |

| | | | | |
|---|---|---|---|---|
| Pr | i-Pr | H | H | —(CH₂)₃— |

| | | | | |
|---|---|---|---|---|
| Pr | Pr | —(CH₂)₃— | H | H |

| | | | | |
|---|---|---|---|---|
| Pr | Pr | H | H | —(CH₂)₃— |

TABLE L114

| | | | | | |
|---|---|---|---|---|---|
| R¹ | R² | R⁵ | R⁶ | R⁷ | R⁸ |

| | | | | |
|---|---|---|---|---|
| Bu | H | —(CH₂)₃— | H | H |

| | | | | |
|---|---|---|---|---|
| Bu | H | H | H | —(CH₂)₃— |

| | | | | |
|---|---|---|---|---|
| Bu | Me | —(CH₂)₃— | H | H |

| | | | | |
|---|---|---|---|---|
| Bu | Me | H | H | —(CH₂)₃— |

| | | | | |
|---|---|---|---|---|
| Bu | Et | —(CH₂)₃— | H | H |

| | | | | |
|---|---|---|---|---|
| Bu | Et | H | H | —(CH₂)₃— |

| | | | | |
|---|---|---|---|---|
| Bu | i-Pr | —(CH₂)₃— | H | H |

| | | | | |
|---|---|---|---|---|
| Bu | i-Pr | H | H | —(CH₂)₃— |

| | | | | |
|---|---|---|---|---|
| Bu | Pr | —(CH₂)₃— | H | H |

| | | | | |
|---|---|---|---|---|
| Bu | Pr | H | H | —(CH₂)₃— |

| | | | | |
|---|---|---|---|---|
| Bu | Bu | —(CH₂)₃— | H | H |

| | | | | |
|---|---|---|---|---|
| Bu | Bu | H | H | —(CH₂)₃— |

| | | | | |
|---|---|---|---|---|
| t-Bu | H | —(CH₂)₃— | H | H |

| | | | | |
|---|---|---|---|---|
| t-Bu | H | H | H | —(CH₂)₃— |

| | | | | |
|---|---|---|---|---|
| t-Bu | Me | —(CH₂)₃— | H | H |

| | | | | |
|---|---|---|---|---|
| t-Bu | Me | H | H | —(CH₂)₃— |

| | | | | |
|---|---|---|---|---|
| t-Bu | Et | —(CH₂)₃— | H | H |

| | | | | |
|---|---|---|---|---|
| t-Bu | Et | H | H | —(CH₂)₃— |

| | | | | |
|---|---|---|---|---|
| t-Bu | i-Pr | —(CH₂)₃— | H | H |

| | | | | |
|---|---|---|---|---|
| t-Bu | i-Pr | H | H | —(CH₂)₃— |

| | | | | |
|---|---|---|---|---|
| t-Bu | Pr | —(CH₂)₃— | H | H |

| | | | | |
|---|---|---|---|---|
| t-Bu | Pr | H | H | —(CH₂)₃— |

| | | | | |
|---|---|---|---|---|
| t-Bu | Bu | —(CH₂)₃— | H | H |

| | | | | |
|---|---|---|---|---|
| t-Bu | Bu | H | H | —(CH₂)₃— |

TABLE L115

| | | | | | |
|---|---|---|---|---|---|
| R¹ | R² | R⁵ | R⁶ | R⁷ | R⁸ |

| | | | | |
|---|---|---|---|---|
| H | H | —(CH₂)₄— | H | H |

| | | | | |
|---|---|---|---|---|
| H | H | H | H | —(CH₂)₄— |

| | | | | |
|---|---|---|---|---|
| Me | H | —(CH₂)₄— | H | H |

| | | | | |
|---|---|---|---|---|
| Me | H | H | H | —(CH₂)₄— |

| | | | | |
|---|---|---|---|---|
| Me | Me | —(CH₂)₄— | H | H |

| | | | | |
|---|---|---|---|---|
| Me | Me | H | H | —(CH₂)₄— |

| | | | | |
|---|---|---|---|---|
| Et | H | —(CH₂)₄— | H | H |

| | | | | |
|---|---|---|---|---|
| Et | H | H | H | —(CH₂)₄— |

| | | | | |
|---|---|---|---|---|
| Et | Me | —(CH₂)₄— | H | H |

| | | | | |
|---|---|---|---|---|
| Et | Me | H | H | —(CH₂)₄— |

| | | | | |
|---|---|---|---|---|
| Et | Et | —(CH₂)₄— | H | H |

| | | | | |
|---|---|---|---|---|
| Et | Et | H | H | —(CH₂)₄— |

| | | | | |
|---|---|---|---|---|
| i-Pr | H | —(CH₂)₄— | H | H |

| | | | | |
|---|---|---|---|---|
| i-Pr | H | H | H | —(CH₂)₄— |

| | | | | |
|---|---|---|---|---|
| i-Pr | Me | —(CH₂)₄— | H | H |

| | | | | |
|---|---|---|---|---|
| i-Pr | Me | H | H | —(CH₂)₄— |

| | | | | |
|---|---|---|---|---|
| i-Pr | Et | —(CH₂)₄— | H | H |

| | | | | |
|---|---|---|---|---|
| i-Pr | Et | H | H | —(CH₂)₄— |

| | | | | |
|---|---|---|---|---|
| Pr | H | —(CH₂)₄— | H | H |

| | | | | |
|---|---|---|---|---|
| Pr | H | H | H | —(CH₂)₄— |

| | | | | |
|---|---|---|---|---|
| Pr | Me | —(CH₂)₄— | H | H |

| | | | | |
|---|---|---|---|---|
| Pr | Me | H | H | —(CH₂)₄— |

| | | | | |
|---|---|---|---|---|
| Pr | Et | —(CH₂)₄— | H | H |

| | | | | |
|---|---|---|---|---|
| Pr | Et | H | H | —(CH₂)₄— |

| | | | | |
|---|---|---|---|---|
| Pr | i-Pr | —(CH₂)₄— | H | H |

| | | | | |
|---|---|---|---|---|
| Pr | i-Pr | H | H | —(CH₂)₄— |

| | | | | |
|---|---|---|---|---|
| Pr | Pr | —(CH₂)₄— | H | H |

| | | | | |
|---|---|---|---|---|
| Pr | Pr | H | H | —(CH₂)₄— |

TABLE L116

| | | | | | |
|---|---|---|---|---|---|
| R¹ | R² | R⁵ | R⁶ | R⁷ | R⁸ |

| | | | | |
|---|---|---|---|---|
| Bu | H | —(CH₂)₄— | H | H |

| | | | | |
|---|---|---|---|---|
| Bu | H | H | H | —(CH₂)₄— |

| | | | | |
|---|---|---|---|---|
| Bu | Me | —(CH₂)₄— | H | H |

| | | | | |
|---|---|---|---|---|
| Bu | Me | H | H | —(CH₂)₄— |

| | | | | |
|---|---|---|---|---|
| Bu | Et | —(CH₂)₄— | H | H |

| | | | | |
|---|---|---|---|---|
| Bu | Et | H | H | —(CH₂)₄— |

| | | | | |
|---|---|---|---|---|
| Bu | i-Pr | —(CH₂)₄— | H | H |

| | | | | |
|---|---|---|---|---|
| Bu | i-Pr | H | H | —(CH₂)₄— |

| | | | | |
|---|---|---|---|---|
| Bu | Pr | —(CH₂)₄— | H | H |

| | | | | |
|---|---|---|---|---|
| Bu | Pr | H | H | —(CH₂)₄— |

| | | | | |
|---|---|---|---|---|
| Bu | Bu | —(CH₂)₄— | H | H |

| | | | | |
|---|---|---|---|---|
| Bu | Bu | H | H | —(CH₂)₄— |

| | | | | |
|---|---|---|---|---|
| t-Bu | H | —(CH₂)₄— | H | H |

| | | | | |
|---|---|---|---|---|
| t-Bu | H | H | H | —(CH₂)₄— |

| | | | | |
|---|---|---|---|---|
| t-Bu | Me | —(CH₂)₄— | H | H |

| | | | | |
|---|---|---|---|---|
| t-Bu | Me | H | H | —(CH₂)₄— |

| | | | | |
|---|---|---|---|---|
| t-Bu | Et | —(CH₂)₄— | H | H |

| | | | | |
|---|---|---|---|---|
| t-Bu | Et | H | H | —(CH₂)₄— |

| | | | | |
|---|---|---|---|---|
| t-Bu | i-Pr | —(CH₂)₄— | H | H |

| | | | | |
|---|---|---|---|---|
| t-Bu | i-Pr | H | H | —(CH₂)₄— |

| | | | | |
|---|---|---|---|---|
| t-Bu | Pr | —(CH₂)₄— | H | H |

| | | | | |
|---|---|---|---|---|
| t-Bu | Pr | H | H | —(CH₂)₄— |

| | | | | |
|---|---|---|---|---|
| t-Bu | Bu | —(CH₂)₄— | H | H |

| | | | | |
|---|---|---|---|---|
| t-Bu | Bu | H | H | —(CH₂)₄— |

TABLE L117

| | | | | | |
|---|---|---|---|---|---|
| R¹ | R² | R⁵ | R⁶ | R⁷ | R⁸ |

| | | | | |
|---|---|---|---|---|
| H | H | —(CH₂)₅— | H | H |

| | | | | |
|---|---|---|---|---|
| H | H | H | H | —(CH₂)₅— |

| | | | | |
|---|---|---|---|---|
| Me | H | —(CH₂)₅— | H | H |

| | | | | |
|---|---|---|---|---|
| Me | H | H | H | —(CH₂)₅— |

| | | | | |
|---|---|---|---|---|
| Me | Me | —(CH₂)₅— | H | H |

| | | | | |
|---|---|---|---|---|
| Me | Me | H | H | —(CH₂)₅— |

| | | | | |
|---|---|---|---|---|
| Et | H | —(CH₂)₅— | H | H |

| | | | | |
|---|---|---|---|---|
| Et | H | H | H | —(CH₂)₅— |

| | | | | |
|---|---|---|---|---|
| Et | Me | —(CH₂)₅— | H | H |

| | | | | |
|---|---|---|---|---|
| Et | Me | H | H | —(CH₂)₅— |

| | | | | |
|---|---|---|---|---|
| Et | Et | —(CH₂)₅— | H | H |

| | | | | |
|---|---|---|---|---|
| Et | Et | H | H | —(CH₂)₅— |

| | | | | |
|---|---|---|---|---|
| i-Pr | H | —(CH₂)₅— | H | H |

| | | | | |
|---|---|---|---|---|
| i-Pr | H | H | H | —(CH₂)₅— |

| | | | | |
|---|---|---|---|---|
| i-Pr | Me | —(CH₂)₅— | H | H |

| | | | | |
|---|---|---|---|---|
| i-Pr | Me | H | H | —(CH₂)₅— |

| | | | | |
|---|---|---|---|---|
| i-Pr | Et | —(CH₂)₅— | H | H |

| | | | | |
|---|---|---|---|---|
| i-Pr | Et | H | H | —(CH₂)₅— |

| | | | | |
|---|---|---|---|---|
| Pr | H | —(CH₂)₅— | H | H |

| | | | | |
|---|---|---|---|---|
| Pr | H | H | H | —(CH₂)₅— |

| | | | | |
|---|---|---|---|---|
| Pr | Me | —(CH₂)₅— | H | H |

| | | | | |
|---|---|---|---|---|
| Pr | Me | H | H | —(CH₂)₅— |

| | | | | |
|---|---|---|---|---|
| Pr | Et | —(CH₂)₅— | H | H |

| | | | | |
|---|---|---|---|---|
| Pr | Et | H | H | —(CH₂)₅— |

| | | | | |
|---|---|---|---|---|
| Pr | i-Pr | —(CH₂)₅— | H | H |

| | | | | |
|---|---|---|---|---|
| Pr | i-Pr | H | H | —(CH₂)₅— |

| | | | | |
|---|---|---|---|---|
| Pr | Pr | —(CH₂)₅— | H | H |

| | | | | |
|---|---|---|---|---|
| Pr | Pr | H | H | —(CH₂)₅— |

TABLE L118

| | | | | | |
|---|---|---|---|---|---|
| R¹ | R² | R⁵ | R⁶ | R⁷ | R⁸ |

| | | | | |
|---|---|---|---|---|
| Bu | H | —(CH₂)₅— | H | H |

| | | | | |
|---|---|---|---|---|
| Bu | H | H | H | —(CH₂)₅— |

| | | | | |
|---|---|---|---|---|
| Bu | Me | —(CH₂)₅— | H | H |

| | | | | |
|---|---|---|---|---|
| Bu | Me | H | H | —(CH₂)₅— |

| | | | | |
|---|---|---|---|---|
| Bu | Et | —(CH₂)₅— | H | H |

| | | | | |
|---|---|---|---|---|
| Bu | Et | H | H | —(CH₂)₅— |

| | | | | |
|---|---|---|---|---|
| Bu | i-Pr | —(CH₂)₅— | H | H |

| | | | | |
|---|---|---|---|---|
| Bu | i-Pr | H | H | —(CH₂)₅— |

| | | | | |
|---|---|---|---|---|
| Bu | Pr | —(CH₂)₅— | H | H |

| | | | | |
|---|---|---|---|---|
| Bu | Pr | H | H | —(CH₂)₅— |

| | | | | |
|---|---|---|---|---|
| Bu | Bu | —(CH₂)₅— | H | H |

| | | | | |
|---|---|---|---|---|
| Bu | Bu | H | H | —(CH₂)₅— |

| | | | | |
|---|---|---|---|---|
| t-Bu | H | —(CH₂)₅— | H | H |

| | | | | |
|---|---|---|---|---|
| t-Bu | H | H | H | —(CH₂)₅— |

| | | | | |
|---|---|---|---|---|
| t-Bu | Me | —(CH₂)₅— | H | H |

| | | | | |
|---|---|---|---|---|
| t-Bu | Me | H | H | —(CH₂)₅— |

| | | | | |
|---|---|---|---|---|
| t-Bu | Et | —(CH₂)₅— | H | H |

| | | | | |
|---|---|---|---|---|
| t-Bu | Et | H | H | —(CH₂)₅— |

| | | | | |
|---|---|---|---|---|
| t-Bu | i-Pr | —(CH₂)₅— | H | H |

| | | | | |
|---|---|---|---|---|
| t-Bu | i-Pr | H | H | —(CH₂)₅— |

| | | | | |
|---|---|---|---|---|
| t-Bu | Pr | —(CH₂)₅— | H | H |

| | | | | |
|---|---|---|---|---|
| t-Bu | Pr | H | H | —(CH₂)₅— |

| | | | | |
|---|---|---|---|---|
| t-Bu | Bu | —(CH₂)₅— | H | H |

| | | | | |
|---|---|---|---|---|
| t-Bu | Bu | H | H | —(CH₂)₅— |

TABLE L119

| R¹ | R² | R⁵ | R⁶ | R⁷ | R⁸ |
|---|---|---|---|---|---|
| CH₂CH═CH₂ | H | Me | Me | H | H |
| CH₂CH═CH₂ | H | H | H | Me | Me |
| CH₂CH═CH₂ | Me | Me | Me | H | H |
| CH₂CH═CH₂ | Me | H | H | Me | Me |
| CH₂CH═CH₂ | Et | Me | Me | H | H |
| CH₂CH═CH₂ | Et | H | H | Me | Me |
| CH₂CH═CH₂ | i-Pr | Me | Me | H | H |
| CH₂CH═CH₂ | i-Pr | H | H | Me | Me |
| CH₂CH═CH₂ | Pr | Me | Me | H | H |
| CH₂CH═CH₂ | Pr | H | H | Me | Me |
| CH₂CH═CH₂ | Bu | Me | Me | H | H |
| CH₂CH═CH₂ | Bu | H | H | Me | Me |
| CH₂CH═CHCH₃ | H | Me | Me | H | H |
| CH₂CH═CHCH₃ | H | H | H | Me | Me |
| CH₂CH═CHCH₃ | Me | Me | Me | H | H |
| CH₂CH═CHCH₃ | Me | H | H | Me | Me |
| CH₂CH═CHCH₃ | Et | Me | Me | H | H |
| CH₂CH═CHCH₃ | Et | H | H | Me | Me |
| CH₂CH═CHCH₃ | i-Pr | Me | Me | H | H |
| CH₂CH═CHCH₃ | i-Pr | H | H | Me | Me |
| CH₂CH═CHCH₃ | Pr | Me | Me | H | H |
| CH₂CH═CHCH₃ | Pr | H | H | Me | Me |
| CH₂CH═CHCH₃ | Bu | Me | Me | H | H |
| CH₂CH═CHCH₃ | Bu | H | H | Me | Me |

TABLE L120

| R¹ | R² | R⁵ | R⁶ | R⁷ | R⁸ |
|---|---|---|---|---|---|
| CH₂CH═C(CH₃)₂ | H | Me | Me | H | H |
| CH₂CH═C(CH₃)₂ | H | H | H | Me | Me |
| CH₂CH═C(CH₃)₂ | Me | Me | Me | H | H |
| CH₂CH═C(CH₃)₂ | Me | H | H | Me | Me |
| CH₂CH═C(CH₃)₂ | Et | Me | Me | H | H |
| CH₂CH═C(CH₃)₂ | Et | H | H | Me | Me |
| CH₂CH═C(CH₃)₂ | i-Pr | Me | Me | H | H |
| CH₂CH═C(CH₃)₂ | i-Pr | H | H | Me | Me |
| CH₂CH═C(CH₃)₂ | Pr | Me | Me | H | H |
| CH₂CH═C(CH₃)₂ | Pr | H | H | Me | Me |
| CH₂CH═C(CH₃)₂ | Bu | Me | Me | H | H |
| CH₂CH═C(CH₃)₂ | Bu | H | H | Me | Me |
| CH₂C(CH₃)═CH₂ | H | Me | Me | H | H |
| CH₂C(CH₃)═CH₂ | H | H | H | Me | Me |
| CH₂C(CH₃)═CH₂ | Me | Me | Me | H | H |
| CH₂C(CH₃)═CH₂ | Me | H | H | Me | Me |
| CH₂C(CH₃)═CH₂ | Et | Me | Me | H | H |
| CH₂C(CH₃)═CH₂ | Et | H | H | Me | Me |
| CH₂C(CH₃)═CH₂ | i-Pr | Me | Me | H | H |
| CH₂C(CH₃)═CH₂ | i-Pr | H | H | Me | Me |
| CH₂C(CH₃)═CH₂ | Pr | Me | Me | H | H |
| CH₂C(CH₃)═CH₂ | Pr | H | H | Me | Me |
| CH₂C(CH₃)═CH₂ | Bu | Me | Me | H | H |
| CH₂C(CH₃)═CH₂ | Bu | H | H | Me | Me |

TABLE L121

| R¹ | R² | R⁵ | R⁶ | R⁷ | R⁸ |
|---|---|---|---|---|---|
| CH₂C(CH₃)═CHCH₃ | H | Me | Me | H | H |
| CH₂C(CH₃)═CHCH₃ | H | H | H | Me | Me |
| CH₂C(CH₃)═CHCH₃ | Me | Me | Me | H | H |
| CH₂C(CH₃)═CHCH₃ | Me | H | H | Me | Me |
| CH₂C(CH₃)═CHCH₃ | Et | Me | Me | H | H |
| CH₂C(CH₃)═CHCH₃ | Et | H | H | Me | Me |
| CH₂C(CH₃)═CHCH₃ | i-Pr | Me | Me | H | H |
| CH₂C(CH₃)═CHCH₃ | i-Pr | H | H | Me | Me |
| CH₂C(CH₃)═CHCH₃ | Pr | Me | Me | H | H |
| CH₂C(CH₃)═CHCH₃ | Pr | H | H | Me | Me |
| CH₂C(CH₃)═CHCH₃ | Bu | Me | Me | H | H |
| CH₂C(CH₃)═CHCH₃ | Bu | H | H | Me | Me |
| CH₂C(CH₃)═C(CH₃)₂ | H | Me | Me | H | H |
| CH₂C(CH₃)═C(CH₃)₂ | H | H | H | Me | Me |
| CH₂C(CH₃)═C(CH₃)₂ | Me | Me | Me | H | H |
| CH₂C(CH₃)═C(CH₃)₂ | Me | H | H | Me | Me |
| CH₂C(CH₃)═C(CH₃)₂ | Et | Me | Me | H | H |
| CH₂C(CH₃)═C(CH₃)₂ | Et | H | H | Me | Me |
| CH₂C(CH₃)═C(CH₃)₂ | i-Pr | Me | Me | H | H |
| CH₂C(CH₃)═C(CH₃)₂ | i-Pr | H | H | Me | Me |
| CH₂C(CH₃)═C(CH₃)₂ | Pr | Me | Me | H | H |
| CH₂C(CH₃)═C(CH₃)₂ | Pr | H | H | Me | Me |
| CH₂C(CH₃)═C(CH₃)₂ | Bu | Me | Me | H | H |
| CH₂C(CH₃)═C(CH₃)₂ | Bu | H | H | Me | Me |

TABLE L122

| R¹ | R² | R⁵ | R⁶ | R⁷ | R⁸ |
|---|---|---|---|---|---|
| CH₂C≡CH | H | Me | Me | H | H |
| CH₂C≡CH | H | H | H | Me | Me |
| CH₂C≡CH | Me | Me | Me | H | H |
| CH₂C≡CH | Me | H | H | Me | Me |
| CH₂C≡CH | Et | Me | Me | H | H |
| CH₂C≡CH | Et | H | H | Me | Me |
| CH₂C≡CH | i-Pr | Me | Me | H | H |
| CH₂C≡CH | i-Pr | H | H | Me | Me |
| CH₂C≡CH | Pr | Me | Me | H | H |
| CH₂C≡CH | Pr | H | H | Me | Me |
| CH₂C≡CH | Bu | Me | Me | H | H |
| CH₂C≡CH | Bu | H | H | Me | Me |
| CH₂C≡CCH₃ | H | Me | Me | H | H |
| CH₂C≡CCH₃ | H | H | H | Me | Me |
| CH₂C≡CCH₃ | Me | Me | Me | H | H |
| CH₂C≡CCH₃ | Me | H | H | Me | Me |
| CH₂C≡CCH₃ | Et | Me | Me | H | H |
| CH₂C≡CCH₃ | Et | H | H | Me | Me |
| CH₂C≡CCH₃ | i-Pr | Me | Me | H | H |
| CH₂C≡CCH₃ | i-Pr | H | H | Me | Me |
| CH₂C≡CCH₃ | Pr | Me | Me | H | H |
| CH₂C≡CCH₃ | Pr | H | H | Me | Me |
| CH₂C≡CCH₃ | Bu | Me | Me | H | H |
| CH₂C≡CCH₃ | Bu | H | H | Me | Me |

TABLE L123

| R¹ | R² | R⁵ | R⁶ | R⁷ | R⁸ |
|---|---|---|---|---|---|
| CH₂C≡CCH₂CH₃ | H | Me | Me | H | H |
| CH₂C≡CCH₂CH₃ | H | H | H | Me | Me |
| CH₂C≡CCH₂CH₃ | Me | Me | Me | H | H |
| CH₂C≡CCH₂CH₃ | Me | H | H | Me | Me |
| CH₂C≡CCH₂CH₃ | Et | Me | Me | H | H |
| CH₂C≡CCH₂CH₃ | Et | H | H | Me | Me |
| CH₂C≡CCH₂CH₃ | i-Pr | Me | Me | H | H |
| CH₂C≡CCH₂CH₃ | i-Pr | H | H | Me | Me |
| CH₂C≡CCH₂CH₃ | Pr | Me | Me | H | H |
| CH₂C≡CCH₂CH₃ | Pr | H | H | Me | Me |
| CH₂C≡CCH₂CH₃ | Bu | Me | Me | H | H |
| CH₂C≡CCH₂CH₃ | Bu | H | H | Me | Me |
| CH₂C≡CCH₂CH₂CH₃ | H | Me | Me | H | H |
| CH₂C≡CCH₂CH₂CH₃ | H | H | H | Me | Me |
| CH₂C≡CCH₂CH₂CH₃ | Me | Me | Me | H | H |
| CH₂C≡CCH₂CH₂CH₃ | Me | H | H | Me | Me |
| CH₂C≡CCH₂CH₂CH₃ | Et | Me | Me | H | H |
| CH₂C≡CCH₂CH₂CH₃ | Et | H | H | Me | Me |
| CH₂C≡CCH₂CH₂CH₃ | i-Pr | Me | Me | H | H |
| CH₂C≡CCH₂CH₂CH₃ | i-Pr | H | H | Me | Me |
| CH₂C≡CCH₂CH₂CH₃ | Pr | Me | Me | H | H |
| CH₂C≡CCH₂CH₂CH₃ | Pr | H | H | Me | Me |
| CH₂C≡CCH₂CH₂CH₃ | Bu | Me | Me | H | H |
| CH₂C≡CCH₂CH₂CH₃ | Bu | H | H | Me | Me |

TABLE L124

| R¹ | R² | R⁵ | R⁶ | R⁷ | R⁸ |
|---|---|---|---|---|---|
| CH₂c-Pr | H | Me | Me | H | H |
| CH₂c-Pr | H | H | H | Me | Me |
| CH₂c-Pr | Me | Me | Me | H | H |
| CH₂c-Pr | Me | H | H | Me | Me |
| CH₂c-Pr | Et | Me | Me | H | H |
| CH₂c-Pr | Et | H | H | Me | Me |
| CH₂c-Pr | i-Pr | Me | Me | H | H |
| CH₂c-Pr | i-Pr | H | H | Me | Me |
| CH₂c-Pr | Pr | Me | Me | H | H |
| CH₂c-Pr | Pr | H | H | Me | Me |
| CH₂c-Pr | Bu | Me | Me | H | H |
| CH₂c-Pr | Bu | H | H | Me | Me |
| CH₂-1-CN-c-Pr | H | Me | Me | H | H |
| CH₂-1-CN-c-Pr | H | H | H | Me | Me |
| CH₂-1-CN-c-Pr | Me | Me | Me | H | H. |
| CH₂-1-CN-c-Pr | Me | H | H | Me | Me |
| CH₂-1-CN-c-Pr | Et | Me | Me | H | H |
| CH₂-1-CN-c-Pr | Et | H | H | Me | Me |
| CH₂-1-CN-c-Pr | i-Pr | Me | Me | H | H |
| CH₂-1-CN-c-Pr | i-Pr | H | H | Me | Me |
| CH₂-1-CN-c-Pr | Pr | Me | Me | H | H |
| CH₂-1-CN-c-Pr | Pr | H | H | Me | Me |
| CH₂-1-CN-c-Pr | Bu | Me | Me | H | H |
| CH₂-1-CN-c-Pr | Bu | H | H | Me | Me |

TABLE L125

| R¹ | R² | R⁵ | R⁶ | R⁷ | R⁸ |
|---|---|---|---|---|---|
| CH₂-1-F-c-Pr | H | Me | Me | H | H |
| CH₂-1-F-c-Pr | H | H | H | Me | Me |
| CH₂-1-F-c-Pr | Me | Me | Me | H | H |
| CH₂-1-F-c-Pr | Me | H | H | Me | Me |
| CH₂-1-F-c-Pr | Et | Me | Me | H | H |
| CH₂-1-F-c-Pr | Et | H | H | Me | Me |
| CH₂-1-F-c-Pr | i-Pr | Me | Me | H | H |
| CH₂-1-F-c-Pr | i-Pr | H | H | Me | Me |
| CH₂-1-F-c-Pr | Pr | Me | Me | H | H |
| CH₂-1-F-c-Pr | Pr | H | H | Me | Me |
| CH₂-1-F-c-Pr | Bu | Me | Me | H | H |
| CH₂-1-F-c-Pr | Bu | H | H | Me | Me |
| CH₂c-Bu | H | Me | Me | H | H |
| CH₂c-Bu | H | H | H | Me | Me |
| CH₂c-Bu | Me | Me | Me | H | H |
| CH₂c-Bu | Me | H | H | Me | Me |
| CH₂c-Bu | Et | Me | Me | H | H |
| CH₂c-Bu | Et | H | H | Me | Me |
| CH₂c-Bu | i-Pr | Me | Me | H | H |
| CH₂c-Bu | i-Pr | H | H | Me | Me |
| CH₂c-Bu | Pr | Me | Me | H | H |
| CH₂c-Bu | Pr | H | H | Me | Me |
| CH₂c-Bu | Bu | Me | Me | H | H |
| CH₂c-Bu | Bu | H | H | Me | Me |

TABLE L126

| R¹ | R² | R⁵ | R⁶ | R⁷ | R⁸ |
|---|---|---|---|---|---|
| CH₂c-C₅H₉ | H | Me | Me | H | H |
| CH₂c-C₅H₉ | H | H | H | Me | Me |
| CH₂c-C₅H₉ | Me | Me | Me | H | H |
| CH₂c-C₅H₉ | Me | H | H | Me | Me |
| CH₂c-C₅H₉ | Et | Me | Me | H | H |
| CH₂c-C₅H₉ | Et | H | H | Me | Me |
| CH₂c-C₅H₉ | i-Pr | Me | Me | H | H |
| CH₂c-C₅H₉ | i-Pr | H | H | Me | Me |
| CH₂c-C₅H₉ | Pr | Me | Me | H | H |
| CH₂c-C₅H₉ | Pr | H | H | Me | Me |
| CH₂c-C₅H₉ | Bu | Me | Me | H | H |
| CH₂c-C₅H₉ | Bu | H | H | Me | Me |
| CH₂-1-cyclopentenyl | H | Me | Me | H | H |
| CH₂-1-cyclopentenyl | H | H | H | Me | Me |
| CH₂-1-cyclopentenyl | Me | Me | Me | H | H |
| CH₂-1-cyclopentenyl | Me | H | H | Me | Me |
| CH₂-1-cyclopentenyl | Et | Me | Me | H | H |
| CH₂-1-cyclopentenyl | Et | H | H | Me | Me |
| CH₂-1-cyclopentenyl | i-Pr | Me | Me | H | H |
| CH₂-1-cyclopentenyl | i-Pr | H | H | Me | Me |
| CH₂-1-cyclopentenyl | Pr | Me | Me | H | H |
| CH₂-1-cyclopentenyl | Pr | H | H | Me | Me |
| CH₂-1-cyclopentenyl | Bu | Me | Me | H | H |
| CH₂-1-cyclopentenyl | Bu | H | H | Me | Me |

TABLE L127

| R¹ | R² | R⁵ | R⁶ | R⁷ | R⁸ |
|---|---|---|---|---|---|
| CH₂c-C₆H₁₁ | H | Me | Me | H | H |
| CH₂c-C₆H₁₁ | H | H | H | Me | Me |
| CH₂c-C₆H₁₁ | Me | Me | Me | H | H |
| CH₂c-C₆H₁₁ | Me | H | H | Me | Me |
| CH₂c-C₆H₁₁ | Et | Me | Me | H | H |
| CH₂c-C₆H₁₁ | Et | H | H | Me | Me |
| CH₂c-C₆H₁₁ | i-Pr | Me | Me | H | H |
| CH₂c-C₆H₁₁ | i-Pr | H | H | Me | Me |
| CH₂c-C₆H₁₁ | Pr | Me | Me | H | H |
| CH₂c-C₆H₁₁ | Pr | H | H | Me | Me |
| CH₂c-C₆H₁₁ | Bu | Me | Me | H | H |
| CH₂c-C₆H₁₁ | Bu | H | H | Me | Me |
| CH₂-1-cyclohexenyl | H | Me | Me | H | H |
| CH₂-1-cyclohexenyl | H | H | H | Me | Me |
| CH₂-1-cyclohexenyl | Me | Me | Me | H | H |
| CH₂-1-cyclohexenyl | Me | H | H | Me | Me |
| CH₂-1-cyclohexenyl | Et | Me | Me | H | H |
| CH₂-1-cyclohexenyl | Et | H | H | Me | Me |
| CH₂-1-cyclohexenyl | i-Pr | Me | Me | H | H |
| CH₂-1-cyclohexenyl | i-Pr | H | H | Me | Me |
| CH₂-1-cyclohexenyl | Pr | Me | Me | H | H |
| CH₂-1-cyclohexenyl | Pr | H | H | Me | Me |
| CH₂-1-cyclohexenyl | Bu | Me | Me | H | H |
| CH₂-1-cyclohexenyl | Bu | H | H | Me | Me |

TABLE L128

| R¹ | R² | R⁵ | R⁶ | R⁷ | R⁸ |
|---|---|---|---|---|---|
| CH₂CH₂OCH₃ | H | Me | Me | H | H |
| CH₂CH₂OCH₃ | H | H | H | Me | Me |
| CH₂CH₂OCH₃ | Me | Me | Me | H | H |
| CH₂CH₂OCH₃ | Me | H | H | Me | Me |
| CH₂CH₂OCH₃ | Et | Me | Me | H | H |
| CH₂CH₂OCH₃ | Et | H | H | Me | Me |
| CH₂CH₂OCH₃ | i-Pr | Me | Me | H | H |
| CH₂CH₂OCH₃ | i-Pr | H | H | Me | Me |
| CH₂CH₂OCH₃ | Pr | Me | Me | H | H |
| CH₂CH₂OCH₃ | Pr | H | H | Me | Me |
| CH₂CH₂OCH₃ | Bu | Me | Me | H | H |
| CH₂CH₂OCH₃ | Bu | H | H | Me | Me |
| CH₂CH₂CH₂OCH₃ | H | Me | Me | H | H |
| CH₂CH₂CH₂OCH₃ | H | H | H | Me | Me |
| CH₂CH₂CH₂OCH₃ | Me | Me | Me | H | H |
| CH₂CH₂CH₂OCH₃ | Me | H | H | Me | Me |
| CH₂CH₂CH₂OCH₃ | Et | Me | Me | H | H |
| CH₂CH₂CH₂OCH₃ | Et | H | H | Me | Me |
| CH₂CH₂CH₂OCH₃ | i-Pr | Me | Me | H | H |
| CH₂CH₂CH₂OCH₃ | i-Pr | H | H | Me | Me |
| CH₂CH₂CH₂OCH₃ | Pr | Me | Me | H | H |
| CH₂CH₂CH₂OCH₃ | Pr | H | H | Me | Me |
| CH₂CH₂CH₂OCH₃ | Bu | Me | Me | H | H |
| CH₂CH₂CH₂OCH₃ | Bu | H | H | Me | Me |

TABLE L129

| R¹ | R² | R⁵ | R⁶ | R⁷ | R⁸ |
|---|---|---|---|---|---|
| CH₂CH₂OCH₂CH₃ | H | Me | Me | H | H |
| CH₂CH₂OCH₂CH₃ | H | H | H | Me | Me |
| CH₂CH₂OCH₂CH₃ | Me | Me | Me | H | H |
| CH₂CH₂OCH₂CH₃ | Me | H | H | Me | Me |
| CH₂CH₂OCH₂CH₃ | Et | Me | Me | H | H |
| CH₂CH₂OCH₂CH₃ | Et | H | H | Me | Me |
| CH₂CH₂OCH₂CH₃ | i-Pr | Me | Me | H | H |
| CH₂CH₂OCH₂CH₃ | i-Pr | H | H | Me | Me |
| CH₂CH₂OCH₂CH₃ | Pr | Me | Me | H | H |
| CH₂CH₂OCH₂CH₃ | Pr | H | H | Me | Me |
| CH₂CH₂OCH₂CH₃ | Bu | Me | Me | H | H |
| CH₂CH₂OCH₂CH₃ | Bu | H | H | Me | Me |
| CH₂CH₂CH₂OCH₂CH₃ | H | Me | Me | H | H |
| CH₂CH₂CH₂OCH₂CH₃ | H | H | H | Me | Me |
| CH₂CH₂CH₂OCH₂CH₃ | Me | Me | Me | H | H |
| CH₂CH₂CH₂OCH₂CH₃ | Me | H | H | Me | Me |
| CH₂CH₂CH₂OCH₂CH₃ | Et | Me | Me | H | H |
| CH₂CH₂CH₂OCH₂CH₃ | Et | H | H | Me | Me |
| CH₂CH₂CH₂OCH₂CH₃ | i-Pr | Me | Me | H | H |
| CH₂CH₂CH₂OCH₂CH₃ | i-Pr | H | H | Me | Me |
| CH₂CH₂CH₂OCH₂CH₃ | Pr | Me | Me | H | H |
| CH₂CH₂CH₂OCH₂CH₃ | Pr | H | H | Me | Me |
| CH₂CH₂CH₂OCH₂CH₃ | Bu | Me | Me | H | H |
| CH₂CH₂CH₂OCH₂CH₃ | Bu | H | H | Me | Me |

TABLE L130

| R¹ | R² | R⁵ | R⁶ | R⁷ | R⁸ |
|---|---|---|---|---|---|
| CH₂CH₂OCH₂CH₂CH₃ | H | Me | Me | H | H |
| CH₂CH₂OCH₂CH₂CH₃ | H | H | H | Me | Me |
| CH₂CH₂OCH₂CH₂CH₃ | Me | Me | Me | H | H |
| CH₂CH₂OCH₂CH₂CH₃ | Me | H | H | Me | Me |
| CH₂CH₂OCH₂CH₂CH₃ | Et | Me | Me | H | H |
| CH₂CH₂OCH₂CH₂CH₃ | Et | H | H | Me | Me |
| CH₂CH₂OCH₂CH₂CH₃ | i-Pr | Me | Me | H | H |
| CH₂CH₂OCH₂CH₂CH₃ | i-Pr | H | H | Me | Me |
| CH₂CH₂OCH₂CH₂CH₃ | Pr | Me | Me | H | H |
| CH₂CH₂OCH₂CH₂CH₃ | Pr | H | H | Me | Me |
| CH₂CH₂OCH₂CH₂CH₃ | Bu | Me | Me | H | H |
| CH₂CH₂OCH₂CH₂CH₃ | Bu | H | H | Me | Me |
| CH₂CH₂OCH₂CH₂CH₃ | H | Me | Me | H | H |
| CH₂CH₂OCH₂CH₂CH₃ | H | H | H | Me | Me |
| CH₂CH₂OCH₂CH₂CH₃ | Me | Me | Me | H | H |
| CH₂CH₂OCH₂CH₂CH₃ | Me | H | H | Me | Me |
| CH₂CH₂OCH₂CH₂CH₃ | Et | Me | Me | H | H |
| CH₂CH₂OCH₂CH₂CH₃ | Et | H | H | Me | Me |
| CH₂CH₂OCH₂CH₂CH₃ | i-Pr | Me | Me | H | H |
| CH₂CH₂OCH₂CH₂CH₃ | i-Pr | H | H | Me | Me |
| CH₂CH₂OCH₂CH₂CH₃ | Pr | Me | Me | H | H |
| CH₂CH₂OCH₂CH₂CH₃ | Pr | H | H | Me | Me |
| CH₂CH₂OCH₂CH₂CH₃ | Bu | Me | Me | H | H |
| CH₂CH₂OCH₂CH₂CH₃ | Bu | H | H | Me | Me |

TABLE L131

| R¹ | R² | R⁵ | R⁶ | R⁷ | R⁸ |
|---|---|---|---|---|---|
| CH₂C(O)CH₃ | H | Me | Me | H | H |
| CH₂C(O)CH₃ | H | H | H | Me | Me |
| CH₂C(O)CH₃ | Me | Me | Me | H | H |
| CH₂C(O)CH₃ | Me | H | H | Me | Me |
| CH₂C(O)CH₃ | Et | Me | Me | H | H |
| CH₂C(O)CH₃ | Et | H | H | Me | Me |
| CH₂C(O)CH₃ | i-Pr | Me | Me | H | H |
| CH₂C(O)CH₃ | i-Pr | H | H | Me | Me |
| CH₂C(O)CH₃ | Pr | Me | Me | H | H |
| CH₂C(O)CH₃ | Pr | H | H | Me | Me |
| CH₂C(O)CH₃ | Bu | Me | Me | H | H |
| CH₂C(O)CH₃ | Bu | H | H | Me | Me |
| CH₂C(O)CH₂CH₃ | H | Me | Me | H | H |
| CH₂C(O)CH₂CH₃ | H | H | H | Me | Me |
| CH₂C(O)CH₂CH₃ | Me | Me | Me | H | H |
| CH₂C(O)CH₂CH₃ | Me | H | H | Me | Me |
| CH₂C(O)CH₂CH₃ | Et | Me | Me | H | H |
| CH₂C(O)CH₂CH₃ | Et | H | H | Me | Me |
| CH₂C(O)CH₂CH₃ | i-Pr | Me | Me | H | H |
| CH₂C(O)CH₂CH₃ | i-Pr | H | H | Me | Me |
| CH₂C(O)CH₂CH₃ | Pr | Me | Me | H | H |
| CH₂C(O)CH₂CH₃ | Pr | H | H | Me | Me |
| CH₂C(O)CH₂CH₃ | Bu | Me | Me | H | H |
| CH₂C(O)CH₂CH₃ | Bu | H | H | Me | Me |

TABLE L132

| R¹ | R² | R⁵ | R⁶ | R⁷ | R⁸ |
|---|---|---|---|---|---|
| CH₂C(O)CF₃ | H | Me | Me | H | H |
| CH₂C(O)CF₃ | H | H | H | Me | Me |
| CH₂C(O)CF₃ | Me | Me | Me | H | H |
| CH₂C(O)CF₃ | Me | H | H | Me | Me |
| CH₂C(O)CF₃ | Et | Me | Me | H | H |
| CH₂C(O)CF₃ | Et | H | H | Me | Me |
| CH₂C(O)CF₃ | i-Pr | Me | Me | H | H |
| CH₂C(O)CF₃ | i-Pr | H | H | Me | Me |
| CH₂C(O)CF₃ | Pr | Me | Me | H | H |
| CH₂C(O)CF₃ | Pr | H | H | Me | Me |
| CH₂C(O)CF₃ | Bu | Me | Me | H | H |
| CH₂C(O)CF₃ | Bu | H | H | Me | Me |
| CH₂C(O)OCH₃ | H | Me | Me | H | H |
| CH₂C(O)OCH₃ | H | H | H | Me | Me |
| CH₂C(O)OCH₃ | Me | Me | Me | H | H |
| CH₂C(O)OCH₃ | Me | H | H | Me | Me |
| CH₂C(O)OCH₃ | Et | Me | Me | H | H |
| CH₂C(O)OCH₃ | Et | H | H | Me | Me |
| CH₂C(O)OCH₃ | i-Pr | Me | Me | H | H |
| CH₂C(O)OCH₃ | i-Pr | H | H | Me | Me |
| CH₂C(O)OCH₃ | Pr | Me | Me | H | H |
| CH₂C(O)OCH₃ | Pr | H | H | Me | Me |
| CH₂C(O)OCH₃ | Bu | Me | Me | H | H |
| CH₂C(O)OCH₃ | Bu | H | H | Me | Me |

TABLE L133

| R¹ | R² | R⁵ | R⁶ | R⁷ | R⁸ |
|---|---|---|---|---|---|
| CH₂C(O)OCH₂CH₃ | H | Me | Me | H | H |
| CH₂C(O)OCH₂CH₃ | H | H | H | Me | Me |
| CH₂C(O)OCH₂CH₃ | Me | Me | Me | H | H |
| CH₂C(O)OCH₂CH₃ | Me | H | H | Me | Me |
| CH₂C(O)OCH₂CH₃ | Et | Me | Me | H | H |
| CH₂C(O)OCH₂CH₃ | Et | H | H | Me | Me |
| CH₂C(O)OCH₂CH₃ | i-Pr | Me | Me | H | H |
| CH₂C(O)OCH₂CH₃ | i-Pr | H | H | Me | Me |
| CH₂C(O)OCH₂CH₃ | Pr | Me | Me | H | H |
| CH₂C(O)OCH₂CH₃ | Pr | H | H | Me | Me |
| CH₂C(O)OCH₂CH₃ | Bu | Me | Me | H | H |
| CH₂C(O)OCH₂CH₃ | Bu | H | H | Me | Me |
| CH₂C(O)OCF₃ | H | Me | Me | H | H |
| CH₂C(O)OCF₃ | H | H | H | Me | Me |
| CH₂C(O)OCF₃ | Me | Me | Me | H | H |
| CH₂C(O)OCF₃ | Me | H | H | Me | Me |
| CH₂C(O)OCF₃ | Et | Me | Me | H | H |
| CH₂C(O)OCF₃ | Et | H | H | Me | Me |
| CH₂C(O)OCF₃ | i-Pr | Me | Me | H | H |
| CH₂C(O)OCF₃ | i-Pr | H | H | Me | Me |
| CH₂C(O)OCF₃ | Pr | Me | Me | H | H |
| CH₂C(O)OCF₃ | Pr | H | H | Me | Me |
| CH₂C(O)OCF₃ | Bu | Me | Me | H | H |
| CH₂C(O)OCF₃ | Bu | H | H | Me | Me |

TABLE L134

| R¹ | R² | R⁵ | R⁶ | R⁷ | R⁸ |
|---|---|---|---|---|---|
| CH₂S(O)₂CH₃ | H | Me | Me | H | H |
| CH₂S(O)₂CH₃ | H | H | H | Me | Me |
| CH₂S(O)₂CH₃ | Me | Me | Me | H | H |
| CH₂S(O)₂CH₃ | Me | H | H | Me | Me |
| CH₂S(O)₂CH₃ | Et | Me | Me | H | H |
| CH₂S(O)₂CH₃ | Et | H | H | Me | Me |
| CH₂S(O)₂CH₃ | i-Pr | Me | Me | H | H |
| CH₂S(O)₂CH₃ | i-Pr | H | H | Me | Me |
| CH₂S(O)₂CH₃ | Pr | Me | Me | H | H |
| CH₂S(O)₂CH₃ | Pr | H | H | Me | Me |
| CH₂S(O)₂CH₃ | Bu | Me | Me | H | H |
| CH₂S(O)₂CH₃ | Bu | H | H | Me | Me |
| CH₂S(O)₂CH₂CH₃ | H | Me | Me | H | H |
| CH₂S(O)₂CH₂CH₃ | H | H | H | Me | Me |
| CH₂S(O)₂CH₂CH₃ | Me | Me | Me | H | H |
| CH₂S(O)₂CH₂CH₃ | Me | H | H | Me | Me |
| CH₂S(O)₂CH₂CH₃ | Et | Me | Me | H | H |
| CH₂S(O)₂CH₂CH₃ | Et | H | H | Me | Me |
| CH₂S(O)₂CH₂CH₃ | i-Pr | Me | Me | H | H |
| CH₂S(O)₂CH₂CH₃ | i-Pr | H | H | Me | Me |
| CH₂S(O)₂CH₂CH₃ | Pr | Me | Me | H | H |
| CH₂S(O)₂CH₂CH₃ | Pr | H | H | Me | Me |
| CH₂S(O)₂CH₂CH₃ | Bu | Me | Me | H | H |
| CH₂S(O)₂CH₂CH₃ | Bu | H | H | Me | Me |

TABLE L135

| R¹ | R² | R⁵ | R⁶ | R⁷ | R⁸ |
|---|---|---|---|---|---|
| CH₂S(O)₂CF₃ | H | Me | Me | H | H |
| CH₂S(O)₂CF₃ | H | H | H | Me | Me |
| CH₂S(O)₂CF₃ | Me | Me | Me | H | H |
| CH₂S(O)₂CF₃ | Me | H | H | Me | Me |
| CH₂S(O)₂CF₃ | Et | Me | Me | H | H |
| CH₂S(O)₂CF₃ | Et | H | H | Me | Me |
| CH₂S(O)₂CF₃ | i-Pr | Me | Me | H | H |
| CH₂S(O)₂CF₃ | i-Pr | H | H | Me | Me |
| CH₂S(O)₂CF₃ | Pr | Me | Me | H | H |
| CH₂S(O)₂CF₃ | Pr | H | H | Me | Me |
| CH₂S(O)₂CF₃ | Bu | Me | Me | H | H |
| CH₂S(O)₂CF₃ | Bu | H | H | Me | Me |
| CH₂CF₃ | H | Me | Me | H | H |
| CH₂CF₃ | H | H | H | Me | Me |
| CH₂CF₃ | Me | Me | Me | H | H. |
| CH₂CF₃ | Me | H | H | Me | Me |
| CH₂CF₃ | Et | Me | Me | H | H |
| CH₂CF₃ | Et | H | H | Me | Me |
| CH₂CF₃ | i-Pr | Me | Me | H | H |
| CH₂CF₃ | i-Pr | H | H | Me | Me |
| CH₂CF₃ | Pr | Me | Me | H | H |
| CH₂CF₃ | Pr | H | H | Me | Me |
| CH₂CF₃ | Bu | Me | Me | H | H |
| CH₂CF₃ | Bu | H | H | Me | Me |

TABLE L136

| R¹ | R² | R⁵ | R⁶ | R⁷ | R⁸ |
|---|---|---|---|---|---|
| CH₂CF₂CF₃ | H | Me | Me | H | H |
| CH₂CF₂CF₃ | H | H | H | Me | Me |
| CH₂CF₂CF₃ | Me | Me | Me | H | H |
| CH₂CF₂CF₃ | Me | H | H | Me | Me |
| CH₂CF₂CF₃ | Et | Me | Me | H | H |
| CH₂CF₂CF₃ | Et | H | H | Me | Me |
| CH₂CF₂CF₃ | i-Pr | Me | Me | H | H |
| CH₂CF₂CF₃ | i-Pr | H | H | Me | Me |
| CH₂CF₂CF₃ | Pr | Me | Me | H | H |
| CH₂CF₂CF₃ | Pr | H | H | Me | Me |
| CH₂CF₂CF₃ | Bu | Me | Me | H | H |
| CH₂CF₂CF₃ | Bu | H | H | Me | Me |
| CH₂CH₂CF₃ | H | Me | Me | H | H |
| CH₂CH₂CF₃ | H | H | H | Me | Me |
| CH₂CH₂CF₃ | Me | Me | Me | H | H |
| CH₂CH₂CF₃ | Me | H | H | Me | Me |
| CH₂CH₂CF₃ | Et | Me | Me | H | H |
| CH₂CH₂CF₃ | Et | H | H | Me | Me |
| CH₂CH₂CF₃ | i-Pr | Me | Me | H | H |
| CH₂CH₂CF₃ | i-Pr | H | H | Me | Me |
| CH₂CH₂CF₃ | Pr | Me | Me | H | H |
| CH₂CH₂CF₃ | Pr | H | H | Me | Me |
| CH₂CH₂CF₃ | Bu | Me | Me | H | H |
| CH₂CH₂CF₃ | Bu | H | H | Me | Me |

TABLE L137

| R¹ | R² | R⁵ | R⁶ | R⁷ | R⁸ |
|---|---|---|---|---|---|
| CH₂CH₂CH₂CF₃ | H | Me | Me | H | H |
| CH₂CH₂CH₂CF₃ | H | H | H | Me | Me |
| CH₂CH₂CH₂CF₃ | Me | Me | Me | H | H |
| CH₂CH₂CH₂CF₃ | Me | H | H | Me | Me |
| CH₂CH₂CH₂CF₃ | Et | Me | Me | H | H |
| CH₂CH₂CH₂CF₃ | Et | H | H | Me | Me |
| CH₂CH₂CH₂CF₃ | i-Pr | Me | Me | H | H |
| CH₂CH₂CH₂CF₃ | i-Pr | H | H | Me | Me |
| CH₂CH₂CH₂CF₃ | Pr | Me | Me | H | H |
| CH₂CH₂CH₂CF₃ | Pr | H | H | Me | Me |
| CH₂CH₂CH₂CF₃ | Bu | Me | Me | H | H |
| CH₂CH₂CH₂CF₃ | Bu | H | H | Me | Me |
| CH₂CH₂CF₂CF₃ | H | Me | Me | H | H |
| CH₂CH₂CF₂CF₃ | H | H | H | Me | Me |
| CH₂CH₂CF₂CF₃ | Me | Me | Me | H | H |
| CH₂CH₂CF₂CF₃ | Me | H | H | Me | Me |
| CH₂CH₂CF₂CF₃ | Et | Me | Me | H | H |
| CH₂CH₂CF₂CF₃ | Et | H | H | Me | Me |
| CH₂CH₂CF₂CF₃ | i-Pr | Me | Me | H | H |
| CH₂CH₂CF₂CF₃ | i-Pr | H | H | Me | Me |
| CH₂CH₂CF₂CF₃ | Pr | Me | Me | H | H |
| CH₂CH₂CF₂CF₃ | Pr | H | H | Me | Me |
| CH₂CH₂CF₂CF₃ | Bu | Me | Me | H | H |
| CH₂CH₂CF₂CF₃ | Bu | H | H | Me | Me |

TABLE L138

| R¹ | R² | R⁵ | R⁶ | R⁷ | R⁸ |
|---|---|---|---|---|---|
| CH₂CCl₃ | H | Me | Me | H | H |
| CH₂CCl₃ | H | H | H | Me | Me |
| CH₂CCl₃ | Me | Me | Me | H | H |
| CH₂CCl₃ | Me | H | H | Me | Me |
| CH₂CCl₃ | Et | Me | Me | H | H |
| CH₂CCl₃ | Et | H | H | Me | Me |
| CH₂CCl₃ | i-Pr | Me | Me | H | H |
| CH₂CCl₃ | i-Pr | H | H | Me | Me |
| CH₂CCl₃ | Pr | Me | Me | H | H |
| CH₂CCl₃ | Pr | H | H | Me | Me |
| CH₂CCl₃ | Bu | Me | Me | H | H |
| CH₂CCl₃ | Bu | H | H | Me | Me |
| CH₂CBr₃ | H | Me | Me | H | H |
| CH₂CBr₃ | H | H | H | Me | Me |
| CH₂CBr₃ | Me | Me | Me | H | H |
| CH₂CBr₃ | Me | H | H | Me | Me |
| CH₂CBr₃ | Et | Me | Me | H | H |
| CH₂CBr₃ | Et | H | H | Me | Me |
| CH₂CBr₃ | i-Pr | Me | Me | H | H |
| CH₂CBr₃ | i-Pr | H | H | Me | Me |
| CH₂CBr₃ | Pr | Me | Me | H | H |
| CH₂CBr₃ | Pr | H | H | Me | Me |
| CH₂CBr₃ | Bu | Me | Me | H | H |
| CH₂CBr₃ | Bu | H | H | Me | Me |

TABLE L139

| R¹ | R² | R⁵ | R⁶ | R⁷ | R⁸ |
|---|---|---|---|---|---|
| CH₂CH₂OH | H | Me | Me | H | H |
| CH₂CH₂OH | H | H | H | Me | Me |
| CH₂CH₂OH | Me | Me | Me | H | H |
| CH₂CH₂OH | Me | H | H | Me | Me |
| CH₂CH₂OH | Et | Me | Me | H | H |
| CH₂CH₂OH | Et | H | H | Me | Me |
| CH₂CH₂OH | i-Pr | Me | Me | H | H |
| CH₂CH₂OH | i-Pr | H | H | Me | Me |
| CH₂CH₂OH | Pr | Me | Me | H | H |
| CH₂CH₂OH | Pr | H | H | Me | Me |
| CH₂CH₂OH | Bu | Me | Me | H | H |
| CH₂CH₂OH | Bu | H | H | Me | Me |
| CH₂CH(OH)CH₃ | H | Me | Me | H | H |
| CH₂CH(OH)CH₃ | H | H | H | Me | Me |
| CH₂CH(OH)CH₃ | Me | Me | Me | H | H |
| CH₂CH(OH)CH₃ | Me | H | H | Me | Me |
| CH₂CH(OH)CH₃ | Et | Me | Me | H | H |
| CH₂CH(OH)CH₃ | Et | H | H | Me | Me |
| CH₂CH(OH)CH₃ | i-Pr | Me | Me | H | H |
| CH₂CH(OH)CH₃ | i-Pr | H | H | Me | Me |
| CH₂CH(OH)CH₃ | Pr | Me | Me | H | H |
| CH₂CH(OH)CH₃ | Pr | H | H | Me | Me |
| CH₂CH(OH)CH₃ | Bu | Me | Me | H | H |
| CH₂CH(OH)CH₃ | Bu | H | H | Me | Me |

TABLE L140

| R¹ | R² | R⁵ | R⁶ | R⁷ | R⁸ |
|---|---|---|---|---|---|
| CH₂CH₂CH₂OH | H | Me | Me | H | H |
| CH₂CH₂CH₂OH | H | H | H | Me | Me |
| CH₂CH₂CH₂OH | Me | Me | Me | H | H |
| CH₂CH₂CH₂OH | Me | H | H | Me | Me |
| CH₂CH₂CH₂OH | Et | Me | Me | H | H |
| CH₂CH₂CH₂OH | Et | H | H | Me | Me |
| CH₂CH₂CH₂OH | i-Pr | Me | Me | H | H |
| CH₂CH₂CH₂OH | i-Pr | H | H | Me | Me |
| CH₂CH₂CH₂OH | Pr | Me | Me | H | H |
| CH₂CH₂CH₂OH | Pr | H | H | Me | Me |
| CH₂CH₂CH₂OH | Bu | Me | Me | H | H |
| CH₂CH₂CH₂OH | Bu | H | H | Me | Me |
| CH₂CN | H | Me | Me | H | H |
| CH₂CN | H | H | H | Me | Me |
| CH₂CN | Me | Me | Me | H | H |
| CH₂CN | Me | H | H | Me | Me |
| CH₂CN | Et | Me | Me | H | H |
| CH₂CN | Et | H | H | Me | Me |
| CH₂CN | i-Pr | Me | Me | H | H |
| CH₂CN | i-Pr | H | H | Me | Me |
| CH₂CN | Pr | Me | Me | H | H |
| CH₂CN | Pr | H | H | Me | Me |
| CH₂CN | Bu | Me | Me | H | H |
| CH₂CN | Bu | H | H | Me | Me |

TABLE L141

| R¹ | R² | R⁵ | R⁶ | R⁷ | R⁸ |
|---|---|---|---|---|---|
| CH(CH₃)CN | H | Me | Me | H | H |
| CH(CH₃)CN | H | H | H | Me | Me |
| CH(CH₃)CN | Me | Me | Me | H | H |
| CH(CH₃)CN | Me | H | H | Me | Me |
| CH(CH₃)CN | Et | Me | Me | H | H |
| CH(CH₃)CN | Et | H | H | Me | Me |
| CH(CH₃)CN | i-Pr | Me | Me | H | H |
| CH(CH₃)CN | i-Pr | H | H | Me | Me |
| CH(CH₃)CN | Pr | Me | Me | H | H |
| CH(CH₃)CN | Pr | H | H | Me | Me |
| CH(CH₃)CN | Bu | Me | Me | H | H |
| CH(CH₃)CN | Bu | H | H | Me | Me |
| CH₂CH₂CN | H | Me | Me | H | H |
| CH₂CH₂CN | H | H | H | Me | Me |
| CH₂CH₂CN | Me | Me | Me | H | H |
| CH₂CH₂CN | Me | H | H | Me | Me |
| CH₂CH₂CN | Et | Me | Me | H | H |
| CH₂CH₂CN | Et | H | H | Me | Me |
| CH₂CH₂CN | i-Pr | Me | Me | H | H |
| CH₂CH₂CN | i-Pr | H | H | Me | Me |
| CH₂CH₂CN | Pr | Me | Me | H | H |
| CH₂CH₂CN | Pr | H | H | Me | Me |
| CH₂CH₂CN | Bu | Me | Me | H | H |
| CH₂CH₂CN | Bu | H | H | Me | Me |

TABLE L142

| R¹ | R² | R⁵ | R⁶ | R⁷ | R⁸ |
|---|---|---|---|---|---|
| CH₂CH(CH₃)CN | H | Me | Me | H | H |
| CH₂CH(CH₃)CN | H | H | H | Me | Me |
| CH₂CH(CH₃)CN | Me | Me | Me | H | H |
| CH₂CH(CH₃)CN | Me | H | H | Me | Me |
| CH₂CH(CH₃)CN | Et | Me | Me | H | H |
| CH₂CH(CH₃)CN | Et | H | H | Me | Me |
| CH₂CH(CH₃)CN | i-Pr | Me | Me | H | H |
| CH₂CH(CH₃)CN | i-Pr | H | H | Me | Me |
| CH₂CH(CH₃)CN | Pr | Me | Me | H | H |
| CH₂CH(CH₃)CN | Pr | H | H | Me | Me |
| CH₂CH(CH₃)CN | Bu | Me | Me | H | H |
| CH₂CH(CH₃)CN | Bu | H | H | Me | Me |
| CH₂C(CH₃)₂CN | H | Me | Me | H | H |
| CH₂C(CH₃)₂CN | H | H | H | Me | Me |
| CH₂C(CH₃)₂CN | Me | Me | Me | H | H |
| CH₂C(CH₃)₂CN | Me | H | H | Me | Me |
| CH₂C(CH₃)₂CN | Et | Me | Me | H | H |
| CH₂C(CH₃)₂CN | Et | H | H | Me | Me |
| CH₂C(CH₃)₂CN | i-Pr | Me | Me | H | H |
| CH₂C(CH₃)₂CN | i-Pr | H | H | Me | Me |
| CH₂C(CH₃)₂CN | Pr | Me | Me | H | H |
| CH₂C(CH₃)₂CN | Pr | H | H | Me | Me |
| CH₂C(CH₃)₂CN | Bu | Me | Me | H | H |
| CH₂C(CH₃)₂CN | Bu | H | H | Me | Me |

TABLE L143

| R¹ | R² | R⁵ | R⁶ | R⁷ | R⁸ |
|---|---|---|---|---|---|
| CH₂C(O)NH₂ | H | Me | Me | H | H |
| CH₂C(O)NH₂ | H | H | H | Me | Me |
| CH₂C(O)NH₂ | Me | Me | Me | H | H |
| CH₂C(O)NH₂ | Me | H | H | Me | Me |
| CH₂C(O)NH₂ | Et | Me | Me | H | H |
| CH₂C(O)NH₂ | Et | H | H | Me | Me |
| CH₂C(O)NH₂ | i-Pr | Me | Me | H | H |
| CH₂C(O)NH₂ | i-Pr | H | H | Me | Me |
| CH₂C(O)NH₂ | Pr | Me | Me | H | H |
| CH₂C(O)NH₂ | Pr | H | H | Me | Me |
| CH₂C(O)NH₂ | Bu | Me | Me | H | H |
| CH₂C(O)NH₂ | Bu | H | H | Me | Me |
| CH₂C(O)NHCH₃ | H | Me | Me | H | H |
| CH₂C(O)NHCH₃ | H | H | H | Me | Me |
| CH₂C(O)NHCH₃ | Me | Me | Me | H | H |
| CH₂C(O)NHCH₃ | Me | H | H | Me | Me |
| CH₂C(O)NHCH₃ | Et | Me | Me | H | H |
| CH₂C(O)NHCH₃ | Et | H | H | Me | Me |
| CH₂C(O)NHCH₃ | i-Pr | Me | Me | H | H |
| CH₂C(O)NHCH₃ | i-Pr | H | H | Me | Me |
| CH₂C(O)NHCH₃ | Pr | Me | Me | H | H |
| CH₂C(O)NHCH₃ | Pr | H | H | Me | Me |
| CH₂C(O)NHCH₃ | Bu | Me | Me | H | H |
| CH₂C(O)NHCH₃ | Bu | H | H | Me | Me |

TABLE L144

| R¹ | R² | R⁵ | R⁶ | R⁷ | R⁸ |
|---|---|---|---|---|---|
| CH₂C(O)N(CH₃)₂ | H | Me | Me | H | H |
| CH₂C(O)N(CH₃)₂ | H | H | H | Me | Me |
| CH₂C(O)N(CH₃)₂ | Me | Me | Me | H | H |
| CH₂C(O)N(CH₃)₂ | Me | H | H | Me | Me |
| CH₂C(O)N(CH₃)₂ | Et | Me | Me | H | H |
| CH₂C(O)N(CH₃)₂ | Et | H | H | Me | Me |
| CH₂C(O)N(CH₃)₂ | i-Pr | Me | Me | H | H |
| CH₂C(O)N(CH₃)₂ | i-Pr | H | H | Me | Me |
| CH₂C(O)N(CH₃)₂ | Pr | Me | Me | H | H |
| CH₂C(O)N(CH₃)₂ | Pr | H | H | Me | Me |
| CH₂C(O)N(CH₃)₂ | Bu | Me | Me | H | H |
| CH₂C(O)N(CH₃)₂ | Bu | H | H | Me | Me |
| OCH₃ | H | Me | Me | H | H |
| OCH₃ | H | H | H | Me | Me |
| OCH₃ | Me | Me | Me | H | H |
| OCH₃ | Me | H | H | Me | Me |
| OCH₃ | Et | Me | Me | H | H |
| OCH₃ | Et | H | H | Me | Me |
| OCH₃ | i-Pr | Me | Me | H | H |
| OCH₃ | i-Pr | H | H | Me | Me |
| OCH₃ | Pr | Me | Me | H | H |
| OCH₃ | Pr | H | H | Me | Me |
| OCH₃ | Bu | Me | Me | H | H |
| OCH₃ | Bu | H | H | Me | Me |

TABLE L145

| R¹ | R² | R⁵ | R⁶ | R⁷ | R⁸ |
|---|---|---|---|---|---|
| OCH₂CH₃ | H | Me | Me | H | H |
| OCH₂CH₃ | H | H | H | Me | Me |
| OCH₂CH₃ | Me | Me | Me | H | H |
| OCH₂CH₃ | Me | H | H | Me | Me |
| OCH₂CH₃ | Et | Me | Me | H | H |
| OCH₂CH₃ | Et | H | H | Me | Me |
| OCH₂CH₃ | i-Pr | Me | Me | H | H |
| OCH₂CH₃ | i-Pr | H | H | Me | Me |
| OCH₂CH₃ | Pr | Me | Me | H | H |
| OCH₂CH₃ | Pr | H | H | Me | Me |
| OCH₂CH₃ | Bu | Me | Me | H | H |
| OCH₂CH₃ | Bu | H | H | Me | Me |
| OCH₂CH₂CH₃ | H | Me | Me | H | H |
| OCH₂CH₂CH₃ | H | H | H | Me | Me |
| OCH₂CH₂CH₃ | Me | Me | Me | H | H |
| OCH₂CH₂CH₃ | Me | H | H | Me | Me |
| OCH₂CH₂CH₃ | Et | Me | Me | H | H |
| OCH₂CH₂CH₃ | Et | H | H | Me | Me |
| OCH₂CH₂CH₃ | i-Pr | Me | Me | H | H |
| OCH₂CH₂CH₃ | i-Pr | H | H | Me | Me |
| OCH₂CH₂CH₃ | Pr | Me | Me | H | H |
| OCH₂CH₂CH₃ | Pr | H | H | Me | Me |
| OCH₂CH₂CH₃ | Bu | Me | Me | H | H |
| OCH₂CH₂CH₃ | Bu | H | H | Me | Me |

TABLE L146

| R¹ | R² | R⁵ | R⁶ | R⁷ | R⁸ |
|---|---|---|---|---|---|
| OCH₂CF₃ | H | Me | Me | H | H |
| OCH₂CF₃ | H | H | H | Me | Me |
| OCH₂CF₃ | Me | Me | Me | H | H |
| OCH₂CF₃ | Me | H | H | Me | Me |
| OCH₂CF₃ | Et | Me | Me | H | H |
| OCH₂CF₃ | Et | H | H | Me | Me |
| OCH₂CF₃ | i-Pr | Me | Me | H | H |
| OCH₂CF₃ | i-Pr | H | H | Me | Me |
| OCH₂CF₃ | Pr | Me | Me | H | H |
| OCH₂CF₃ | Pr | H | H | Me | Me |
| OCH₂CF₃ | Bu | Me | Me | H | H |
| OCH₂CF₃ | Bu | H | H | Me | Me |
| OCH₂CH₂CF₃ | H | Me | Me | H | H |
| OCH₂CH₂CF₃ | H | H | H | Me | Me |
| OCH₂CH₂CF₃ | Me | Me | Me | H | H |
| OCH₂CH₂CF₃ | Me | H | H | Me | Me |
| OCH₂CH₂CF₃ | Et | Me | Me | H | H |
| OCH₂CH₂CF₃ | Et | H | H | Me | Me |
| OCH₂CH₂CF₃ | i-Pr | Me | Me | H | H |
| OCH₂CH₂CF₃ | i-Pr | H | H | Me | Me |
| OCH₂CH₂CF₃ | Pr | Me | Me | H | H |
| OCH₂CH₂CF₃ | Pr | H | H | Me | Me |
| OCH₂CH₂CF₃ | Bu | Me | Me | H | H |
| OCH₂CH₂CF₃ | Bu | H | H | Me | Me |

TABLE L147

| R¹ | R² | R⁵ | R⁶ | R⁷ | R⁸ |
|---|---|---|---|---|---|
| C(O)CH₃ | H | Me | Me | H | H |
| C(O)CH₃ | H | H | H | Me | Me |
| C(O)CH₃ | Me | Me | Me | H | H |
| C(O)CH₃ | Me | H | H | Me | Me |
| C(O)CH₃ | Et | Me | Me | H | H |
| C(O)CH₃ | Et | H | H | Me | Me |
| C(O)CH₃ | i-Pr | Me | Me | H | H |
| C(O)CH₃ | i-Pr | H | H | Me | Me |
| C(O)CH₃ | Pr | Me | Me | H | H |
| C(O)CH₃ | Pr | H | H | Me | Me |
| C(O)CH₃ | Bu | Me | Me | H | H |
| C(O)CH₃ | Bu | H | H | Me | Me |
| C(O)CH₂CH₃ | H | Me | Me | H | H |
| C(O)CH₂CH₃ | H | H | H | Me | Me |
| C(O)CH₂CH₃ | Me | Me | Me | H | H |
| C(O)CH₂CH₃ | Me | H | H | Me | Me |
| C(O)CH₂CH₃ | Et | Me | Me | H | H |
| C(O)CH₂CH₃ | Et | H | H | Me | Me |
| C(O)CH₂CH₃ | i-Pr | Me | Me | H | H |
| C(O)CH₂CH₃ | i-Pr | H | H | Me | Me |
| C(O)CH₂CH₃ | Pr | Me | Me | H | H |
| C(O)CH₂CH₃ | Pr | H | H | Me | Me |
| C(O)CH₂CH₃ | Bu | Me | Me | H | H |
| C(O)CH₂CH₃ | Bu | H | H | Me | Me |

TABLE L148

| R¹ | R² | R⁵ | R⁶ | R⁷ | R⁸ |
|---|---|---|---|---|---|
| C(O)CF₃ | H | Me | Me | H | H |
| C(O)CF₃ | H | H | H | Me | Me |
| C(O)CF₃ | Me | Me | Me | H | H |
| C(O)CF₃ | Me | H | H | Me | Me |
| C(O)CF₃ | Et | Me | Me | H | H |
| C(O)CF₃ | Et | H | H | Me | Me |
| C(O)CF₃ | i-Pr | Me | Me | H | H |
| C(O)CF₃ | i-Pr | H | H | Me | Me |
| C(O)CF₃ | Pr | Me | Me | H | H |
| C(O)CF₃ | Pr | H | H | Me | Me |
| C(O)CF₃ | Bu | Me | Me | H | H |
| C(O)CF₃ | Bu | H | H | Me | Me |
| C(O)CH₂CF₃ | H | Me | Me | H | H |
| C(O)CH₂CF₃ | H | H | H | Me | Me |
| C(O)CH₂CF₃ | Me | Me | Me | H | H |
| C(O)CH₂CF₃ | Me | H | H | Me | Me |
| C(O)CH₂CF₃ | Et | Me | Me | H | H |
| C(O)CH₂CF₃ | Et | H | H | Me | Me |
| C(O)CH₂CF₃ | i-Pr | Me | Me | H | H |
| C(O)CH₂CF₃ | i-Pr | H | H | Me | Me |
| C(O)CH₂CF₃ | Pr | Me | Me | H | H |
| C(O)CH₂CF₃ | Pr | H | H | Me | Me |
| C(O)CH₂CF₃ | Bu | Me | Me | H | H |
| C(O)CH₂CF₃ | Bu | H | H | Me | Me |

TABLE L149

| R¹ | R² | R⁵ | R⁶ | R⁷ | R⁸ |
|---|---|---|---|---|---|
| C(O)OCH₃ | H | Me | Me | H | H |
| C(O)OCH₃ | H | H | H | Me | Me |
| C(O)OCH₃ | Me | Me | Me | H | H |
| C(O)OCH₃ | Me | H | H | Me | Me |
| C(O)OCH₃ | Et | Me | Me | H | H |
| C(O)OCH₃ | Et | H | H | Me | Me |
| C(O)OCH₃ | i-Pr | Me | Me | H | H |
| C(O)OCH₃ | i-Pr | H | H | Me | Me |
| C(O)OCH₃ | Pr | Me | Me | H | H |
| C(O)OCH₃ | Pr | H | H | Me | Me |
| C(O)OCH₃ | Bu | Me | Me | H | H |
| C(O)OCH₃ | Bu | H | H | Me | Me |
| C(O)OCH₂CH₃ | H | Me | Me | H | H |
| C(O)OCH₂CH₃ | H | H | H | Me | Me |
| C(O)OCH₂CH₃ | Me | Me | Me | H | H |
| C(O)OCH₂CH₃ | Me | H | H | Me | Me |
| C(O)OCH₂CH₃ | Et | Me | Me | H | H |
| C(O)OCH₂CH₃ | Et | H | H | Me | Me |
| C(O)OCH₂CH₃ | i-Pr | Me | Me | H | H |
| C(O)OCH₂CH₃ | i-Pr | H | H | Me | Me |
| C(O)OCH₂CH₃ | Pr | Me | Me | H | H |
| C(O)OCH₂CH₃ | Pr | H | H | Me | Me |
| C(O)OCH₂CH₃ | Bu | Me | Me | H | H |
| C(O)OCH₂CH₃ | Bu | H | H | Me | Me |

TABLE L150

| R¹ | R² | R⁵ | R⁶ | R⁷ | R⁸ |
|---|---|---|---|---|---|
| C(O)OCF₃ | H | Me | Me | H | H |
| C(O)OCF₃ | H | H | H | Me | Me |
| C(O)OCF₃ | Me | Me | Me | H | H |
| C(O)OCF₃ | Me | H | H | Me | Me |
| C(O)OCF₃ | Et | Me | Me | H | H |
| C(O)OCF₃ | Et | H | H | Me | Me |
| C(O)OCF₃ | i-Pr | Me | Me | H | H |
| C(O)OCF₃ | i-Pr | H | H | Me | Me |
| C(O)OCF₃ | Pr | Me | Me | H | H |
| C(O)OCF₃ | Pr | H | H | Me | Me |
| C(O)OCF₃ | Bu | Me | Me | H | H |
| C(O)OCF₃ | Bu | H | H | Me | Me |
| C(O)OCH₂CF₃ | H | Me | Me | H | H |
| C(O)OCH₂CF₃ | H | H | H | Me | Me |
| C(O)OCH₂CF₃ | Me | Me | Me | H | H |
| C(O)OCH₂CF₃ | Me | H | H | Me | Me |
| C(O)OCH₂CF₃ | Et | Me | Me | H | H |
| C(O)OCH₂CF₃ | Et | H | H | Me | Me |
| C(O)OCH₂CF₃ | i-Pr | Me | Me | H | H |
| C(O)OCH₂CF₃ | i-Pr | H | H | Me | Me |
| C(O)OCH₂CF₃ | Pr | Me | Me | H | H |
| C(O)OCH₂CF₃ | Pr | H | H | Me | Me |
| C(O)OCH₂CF₃ | Bu | Me | Me | H | H |
| C(O)OCH₂CF₃ | Bu | H | H | Me | Me |

TABLE L151

| R¹ | R² | R⁵ | R⁶ | R⁷ | R⁸ |
|---|---|---|---|---|---|
| S(O)₂CH₃ | H | Me | Me | H | H |
| S(O)₂CH₃ | H | H | H | Me | Me |
| S(O)₂CH₃ | Me | Me | Me | H | H |
| S(O)₂CH₃ | Me | H | H | Me | Me |
| S(O)₂CH₃ | Et | Me | Me | H | H |
| S(O)₂CH₃ | Et | H | H | Me | Me |
| S(O)₂CH₃ | i-Pr | Me | Me | H | H |
| S(O)₂CH₃ | i-Pr | H | H | Me | Me |
| S(O)₂CH₃ | Pr | Me | Me | H | H |
| S(O)₂CH₃ | Pr | H | H | Me | Me |
| S(O)₂CH₃ | Bu | Me | Me | H | H |
| S(O)₂CH₃ | Bu | H | H | Me | Me |
| S(O)₂CH₂CH₃ | H | Me | Me | H | H |
| S(O)₂CH₂CH₃ | H | H | H | Me | Me |
| S(O)₂CH₂CH₃ | Me | Me | Me | H | H |
| S(O)₂CH₂CH₃ | Me | H | H | Me | Me |
| S(O)₂CH₂CH₃ | Et | Me | Me | H | H |
| S(O)₂CH₂CH₃ | Et | H | H | Me | Me |
| S(O)₂CH₂CH₃ | i-Pr | Me | Me | H | H |
| S(O)₂CH₂CH₃ | i-Pr | H | H | Me | Me |
| S(O)₂CH₂CH₃ | Pr | Me | Me | H | H |
| S(O)₂CH₂CH₃ | Pr | H | H | Me | Me |
| S(O)₂CH₂CH₃ | Bu | Me | Me | H | H |
| S(O)₂CH₂CH₃ | Bu | H | H | Me | Me |

TABLE L152

| R¹ | R² | R⁵ | R⁶ | R⁷ | R⁸ |
|---|---|---|---|---|---|
| S(O)₂CF₃ | H | Me | Me | H | H |
| S(O)₂CF₃ | H | H | H | Me | Me |
| S(O)₂CF₃ | Me | Me | Me | H | H |
| S(O)₂CF₃ | Me | H | H | Me | Me |
| S(O)₂CF₃ | Et | Me | Me | H | H |
| S(O)₂CF₃ | Et | H | H | Me | Me |
| S(O)₂CF₃ | i-Pr | Me | Me | H | H |
| S(O)₂CF₃ | i-Pr | H | H | Me | Me |
| S(O)₂CF₃ | Pr | Me | Me | H | H |
| S(O)₂CF₃ | Pr | H | H | Me | Me |
| S(O)₂CF₃ | Bu | Me | Me | H | H |
| S(O)₂CF₃ | Bu | H | H | Me | Me |
| S(O)₂CH₂CF₃ | H | Me | Me | H | H |
| S(O)₂CH₂CF₃ | H | H | H | Me | Me |
| S(O)₂CH₂CF₃ | Me | Me | Me | H | H |
| S(O)₂CH₂CF₃ | Me | H | H | Me | Me |
| S(O)₂CH₂CF₃ | Et | Me | Me | H | H |
| S(O)₂CH₂CF₃ | Et | H | H | Me | Me |
| S(O)₂CH₂CF₃ | i-Pr | Me | Me | H | H |
| S(O)₂CH₂CF₃ | i-Pr | H | H | Me | Me |
| S(O)₂CH₂CF₃ | Pr | Me | Me | H | H |
| S(O)₂CH₂CF₃ | Pr | H | H | Me | Me |
| S(O)₂CH₂CF₃ | Bu | Me | Me | H | H |
| S(O)₂CH₂CF₃ | Bu | H | H | Me | Me |

TABLE L153

| | | | | | |
|---|---|---|---|---|---|
| R¹ | R² | R⁵ | R⁶ | R⁷ | R⁸ |

| | | | | |
|---|---|---|---|---|
| —CH₂CH₂— | Me | Me | H | H |
| —CH₂CH₂— | H | H | Me | Me |
| —CH₂CH₂CH₂— | Me | Me | H | H |
| —CH₂CH₂CH₂— | H | H | Me | Me |
| —CH₂CH(CH₃)CH₂— | Me | Me | H | H |
| —CH₂CH(CH₃)CH₂— | H | H | Me | Me |
| —CH₂C(CH₃)₂CH₂— | Me | Me | H | H |
| —CH₂C(CH₃)₂CH₂— | H | H | Me | Me |
| —CH₂CH₂CH₂CH₂— | Me | Me | H | H |
| —CH₂CH₂CH₂CH₂— | H | H | Me | Me |
| —CH₂CH(CH₃)CH₂CH₂— | Me | Me | H | H |
| —CH₂CH(CH₃)CH₂CH₂— | H | H | Me | Me |
| —CH₂C(CH₃)₂CH₂CH₂— | Me | Me | H | H |
| —CH₂C(CH₃)₂CH₂CH₂— | H | H | Me | Me |
| —CH₂CH₂CH₂CH₂CH₂— | Me | Me | H | H |
| —CH₂CH₂CH₂CH₂CH₂— | H | H | Me | Me |
| —CH₂CH₂OCH₂CH₂— | Me | Me | H | H |
| —CH₂CH₂OCH₂CH₂— | H | H | Me | Me |
| —CH₂CH₂SCH₂CH₂— | Me | Me | H | H |
| —CH₂CH₂SCH₂CH₂— | H | H | Me | Me |
| —CH₂CH₂NHCH₂CH₂— | Me | Me | H | H |
| —CH₂CH₂NHCH₂CH₂— | H | H | Me | Me |
| —CH₂CH₂N(CH₃)CH₂CH₂— | Me | Me | H | H |
| —CH₂CH₂N(CH₃)CH₂CH₂— | H | H | Me | Me |

TABLE L154

| R¹ | R² | R⁵ | R⁶ | R⁷ | R⁸ |
|---|---|---|---|---|---|
| CH₂—Ph | H | Me | Me | H | H |
| CH₂—Ph | H | H | H | Me | Me |
| CH₂—Ph | Me | Me | Me | H | H |
| CH₂—Ph | Me | H | H | Me | Me |
| CH₂—Ph | Et | Me | Me | H | H |
| CH₂—Ph | Et | H | H | Me | Me |
| CH₂—Ph | i-Pr | Me | Me | H | H |
| CH₂—Ph | i-Pr | H | H | Me | Me |
| CH₂—Ph | Pr | Me | Me | H | H |
| CH₂—Ph | Pr | H | H | Me | Me |
| CH₂—Ph | Bu | Me | Me | H | H |
| CH₂—Ph | Bu | H | H | Me | Me |
| CH₂—2—CH₃—Ph | H | Me | Me | H | H |
| CH₂—2—CH₃—Ph | H | H | H | Me | Me |
| CH₂—2—CH₃—Ph | Me | Me | Me | H | H |
| CH₂—2—CH₃—Ph | Me | H | H | Me | Me |
| CH₂—2—CH₃—Ph | Et | Me | Me | H | H |
| CH₂—2—CH₃—Ph | Et | H | H | Me | Me |
| CH₂—2—CH₃—Ph | i-Pr | Me | Me | H | H |
| CH₂—2—CH₃—Ph | i-Pr | H | H | Me | Me |
| CH₂—2—CH₃—Ph | Pr | Me | Me | H | H |
| CH₂—2—CH₃—Ph | Pr | H | H | Me | Me |
| CH₂—2—CH₃—Ph | Bu | Me | Me | H | H |
| CH₂—2—CH₃—Ph | Bu | H | H | Me | Me |

TABLE L155

| R¹ | R² | R⁵ | R⁶ | R⁷ | R⁸ |
|---|---|---|---|---|---|
| CH₂—3—CH₃—Ph | H | Me | Me | H | H |
| CH₂—3—CH₃—Ph | H | H | H | Me | Me |
| CH₂—3—CH₃—Ph | Me | Me | Me | H | H |
| CH₂—3—CH₃—Ph | Me | H | H | Me | Me |
| CH₂—3—CH₃—Ph | Et | Me | Me | H | H |
| CH₂—3—CH₃—Ph | Et | H | H | Me | Me |
| CH₂—3—CH₃—Ph | i-Pr | Me | Me | H | H |
| CH₂—3—CH₃—Ph | i-Pr | H | H | Me | Me |
| CH₂—3—CH₃—Ph | Pr | Me | Me | H | H |
| CH₂—3—CH₃—Ph | Pr | H | H | Me | Me |
| CH₂—3—CH₃—Ph | Bu | Me | Me | H | H |
| CH₂—3—CH₃—Ph | Bu | H | H | Me | Me |
| CH₂—4—CH₃—Ph | H | Me | Me | H | H |
| CH₂—4—CH₃—Ph | H | H | H | Me | Me |
| CH₂—4—CH₃—Ph | Me | Me | Me | H | H |
| CH₂—4—CH₃—Ph | Me | H | H | Me | Me |
| CH₂—4—CH₃—Ph | Et | Me | Me | H | H |
| CH₂—4—CH₃—Ph | Et | H | H | Me | Me |
| CH₂—4—CH₃—Ph | i-Pr | Me | Me | H | H |
| CH₂—4—CH₃—Ph | i-Pr | H | H | Me | Me |
| CH₂—4—CH₃—Ph | Pr | Me | Me | H | H |
| CH₂—4—CH₃—Ph | Pr | H | H | Me | Me |
| CH₂—4—CH₃—Ph | Bu | Me | Me | H | H |
| CH₂—4—CH₃—Ph | Bu | H | H | Me | Me |

TABLE L156

| R¹ | R² | R⁵ | R⁶ | R⁷ | R⁸ |
|---|---|---|---|---|---|
| CH₂—2—OCH₃—Ph | H | Me | Me | H | H |
| CH₂—2—OCH₃—Ph | H | H | H | Me | Me |
| CH₂—2—OCH₃—Ph | Me | Me | Me | H | H |
| CH₂—2—OCH₃—Ph | Me | H | H | Me | Me |
| CH₂—2—OCH₃—Ph | Et | Me | Me | H | H |
| CH₂—2—OCH₃—Ph | Et | H | H | Me | Me |
| CH₂—2—OCH₃—Ph | i-Pr | Me | Me | H | H |
| CH₂—2—OCH₃—Ph | i-Pr | H | H | Me | Me |
| CH₂—2—OCH₃—Ph | Pr | Me | Me | H | H |
| CH₂—2—OCH₃—Ph | Pr | H | H | Me | Me |
| CH₂—2—OCH₃—Ph | Bu | Me | Me | H | H |
| CH₂—2—OCH₃—Ph | Bu | H | H | Me | Me |
| CH₂—3—OCH₃—Ph | H | Me | Me | H | H |
| CH₂—3—OCH₃—Ph | H | H | H | Me | Me |
| CH₂—3—OCH₃—Ph | Me | Me | Me | H | H |
| CH₂—3—OCH₃—Ph | Me | H | H | Me | Me |
| CH₂—3—OCH₃—Ph | Et | Me | Me | H | H |
| CH₂—3—OCH₃—Ph | Et | H | H | Me | Me |
| CH₂—3—OCH₃—Ph | i-Pr | Me | Me | H | H |
| CH₂—3—OCH₃—Ph | i-Pr | H | H | Me | Me |
| CH₂—3—OCH₃—Ph | Pr | Me | Me | H | H |
| CH₂—3—OCH₃—Ph | Pr | H | H | Me | Me |
| CH₂—3—OCH₃—Ph | Bu | Me | Me | H | H |
| CH₂—3—OCH₃—Ph | Bu | H | H | Me | Me |

TABLE L157

| R¹ | R² | R⁵ | R⁶ | R⁷ | R⁸ |
|---|---|---|---|---|---|
| CH₂—4—OCH₃—Ph | H | Me | Me | H | H |
| CH₂—4—OCH₃—Ph | H | H | H | Me | Me |
| CH₂—4—OCH₃—Ph | Me | Me | Me | H | H |
| CH₂—4—OCH₃—Ph | Me | H | H | Me | Me |
| CH₂—4—OCH₃—Ph | Et | Me | Me | H | H |
| CH₂—4—OCH₃—Ph | Et | H | H | Me | Me |
| CH₂—4—OCH₃—Ph | i-Pr | Me | Me | H | H |
| CH₂—4—OCH₃—Ph | i-Pr | H | H | Me | Me |
| CH₂—4—OCH₃—Ph | Pr | Me | Me | H | H |
| CH₂—4—OCH₃—Ph | Pr | H | H | Me | Me |
| CH₂—4—OCH₃—Ph | Bu | Me | Me | H | H |
| CH₂—4—OCH₃—Ph | Bu | H | H | Me | Me |
| CH₂—2—F—Ph | H | Me | Me | H | H |
| CH₂—2—F—Ph | H | H | H | Me | Me |
| CH₂—2—F—Ph | Me | Me | Me | H | H |
| CH₂—2—F—Ph | Me | H | H | Me | Me |
| CH₂—2—F—Ph | Et | Me | Me | H | H |
| CH₂—2—F—Ph | Et | H | H | Me | Me |
| CH₂—2—F—Ph | i-Pr | Me | Me | H | H |
| CH₂—2—F—Ph | i-Pr | H | H | Me | Me |
| CH₂—2—F—Ph | Pr | Me | Me | H | H |
| CH₂—2—F—Ph | Pr | H | H | Me | Me |
| CH₂—2—F—Ph | Bu | Me | Me | H | H |
| CH₂—2—F—Ph | Bu | H | H | Me | Me |

TABLE L158

| R¹ | R² | R⁵ | R⁶ | R⁷ | R⁸ |
|---|---|---|---|---|---|
| CH₂-3-F—Ph | H | Me | Me | H | H |
| CH₂-3-F—Ph | H | H | H | Me | Me |
| CH₂-3-F—Ph | Me | Me | Me | H | H |
| CH₂-3-F—Ph | Me | H | H | Me | Me |
| CH₂-3-F—Ph | Et | Me | Me | H | H |
| CH₂-3-F—Ph | Et | H | H | Me | Me |
| CH₂-3-F—Ph | i-Pr | Me | Me | H | H |
| CH₂-3-F—Ph | i-Pr | H | H | Me | Me |
| CH₂-3-F—Ph | Pr | Me | Me | H | H |
| CH₂-3-F—Ph | Pr | H | H | Me | Me |
| CH₂-3-F—Ph | Bu | Me | Me | H | H |
| CH₂-3-F—Ph | Bu | H | H | Me | Me |
| CH₂-4-F—Ph | H | Me | Me | H | H |
| CH₂-4-F—Ph | H | H | H | Me | Me |
| CH₂-4-F—Ph | Me | Me | Me | H | H |
| CH₂-4-F—Ph | Me | H | H | Me | Me |
| CH₂-4-F—Ph | Et | Me | Me | H | H |
| CH₂-4-F—Ph | Et | H | H | Me | Me |
| CH₂-4-F—Ph | i-Pr | Me | Me | H | H |
| CH₂-4-F—Ph | i-Pr | H | H | Me | Me |
| CH₂-4-F—Ph | Pr | Me | Me | H | H |
| CH₂-4-F—Ph | Pr | H | H | Me | Me |
| CH₂-4-F—Ph | Bu | Me | Me | H | H |
| CH₂-4-F—Ph | Bu | H | H | Me | Me |

TABLE L159

| R¹ | R² | R⁵ | R⁶ | R⁷ | R⁸ |
|---|---|---|---|---|---|
| CH₂-2-Cl—Ph | H | Me | Me | H | H |
| CH₂-2-Cl—Ph | H | H | H | Me | Me |
| CH₂-2-Cl—Ph | Me | Me | Me | H | H |
| CH₂-2-Cl—Ph | Me | H | H | Me | Me |
| CH₂-2-Cl—Ph | Et | Me | Me | H | H |
| CH₂-2-Cl—Ph | Et | H | H | Me | Me |
| CH₂-2-Cl—Ph | i-Pr | Me | Me | H | H |
| CH₂-2-Cl—Ph | i-Pr | H | H | Me | Me |
| CH₂-2-Cl—Ph | Pr | Me | Me | H | H |
| CH₂-2-Cl—Ph | Pr | H | H | Me | Me |
| CH₂-2-Cl—Ph | Bu | Me | Me | H | H |
| CH₂-2-Cl—Ph | Bu | H | H | Me | Me |
| CH₂-3-Cl—Ph | H | Me | Me | H | H |
| CH₂-3-Cl—Ph | H | H | H | Me | Me |
| CH₂-3-Cl—Ph | Me | Me | Me | H | H |
| CH₂-3-Cl—Ph | Me | H | H | Me | Me |
| CH₂-3-Cl—Ph | Et | Me | Me | H | H |
| CH₂-3-Cl—Ph | Et | H | H | Me | Me |
| CH₂-3-Cl—Ph | i-Pr | Me | Me | H | H |
| CH₂-3-Cl—Ph | i-Pr | H | H | Me | Me |
| CH₂-3-Cl—Ph | Pr | Me | Me | H | H |
| CH₂-3-Cl—Ph | Pr | H | H | Me | Me |
| CH₂-3-Cl—Ph | Bu | Me | Me | H | H |
| CH₂-3-Cl—Ph | Bu | H | H | Me | Me |

TABLE L160

| R¹ | R² | R⁵ | R⁶ | R⁷ | R⁸ |
|---|---|---|---|---|---|
| CH₂-4-Cl—Ph | H | Me | Me | H | H |
| CH₂-4-Cl—Ph | H | H | H | Me | Me |
| CH₂-4-Cl—Ph | Me | Me | Me | H | H |
| CH₂-4-Cl—Ph | Me | H | H | Me | Me |
| CH₂-4-Cl—Ph | Et | Me | Me | H | H |
| CH₂-4-Cl—Ph | Et | H | H | Me | Me |
| CH₂-4-Cl—Ph | i-Pr | Me | Me | H | H |
| CH₂-4-Cl—Ph | i-Pr | H | H | Me | Me |
| CH₂-4-Cl—Ph | Pr | Me | Me | H | H |
| CH₂-4-Cl—Ph | Pr | H | H | Me | Me |
| CH₂-4-Cl—Ph | Bu | Me | Me | H | H |
| CH₂-4-Cl—Ph | Bu | H | H | Me | Me |
| CH₂-2-Br—Ph | H | Me | Me | H | H |
| CH₂-2-Br—Ph | H | H | H | Me | Me |
| CH₂-2-Br—Ph | Me | Me | Me | H | H |
| CH₂-2-Br—Ph | Me | H | H | Me | Me |
| CH₂-2-Br—Ph | Et | Me | Me | H | H |
| CH₂-2-Br—Ph | Et | H | H | Me | Me |
| CH₂-2-Br—Ph | i-Pr | Me | Me | H | H |
| CH₂-2-Br—Ph | i-Pr | H | H | Me | Me |
| CH₂-2-Br—Ph | Pr | Me | Me | H | H |
| CH₂-2-Br—Ph | Pr | H | H | Me | Me |
| CH₂-2-Br—Ph | Bu | Me | Me | H | H |
| CH₂-2-Br—Ph | Bu | H | H | Me | Me |

TABLE L161

| R¹ | R² | R⁵ | R⁶ | R⁷ | R⁸ |
|---|---|---|---|---|---|
| CH₂-3-Br—Ph | H | Me | Me | H | H |
| CH₂-3-Br—Ph | H | H | H | Me | Me |
| CH₂-3-Br—Ph | Me | Me | Me | H | H |
| CH₂-3-Br—Ph | Me | H | H | Me | Me |
| CH₂-3-Br—Ph | Et | Me | Me | H | H |
| CH₂-3-Br—Ph | Et | H | H | Me | Me |
| CH₂-3-Br—Ph | i-Pr | Me | Me | H | H |
| CH₂-3-Br—Ph | i-Pr | H | H | Me | Me |
| CH₂-3-Br—Ph | Pr | Me | Me | H | H |
| CH₂-3-Br—Ph | Pr | H | H | Me | Me |
| CH₂-3-Br—Ph | Bu | Me | Me | H | H |
| CH₂-3-Br—Ph | Bu | H | H | Me | Me |
| CH₂-4-Br—Ph | H | Me | Me | H | H |
| CH₂-4-Br—Ph | H | H | H | Me | Me |
| CH₂-4-Br—Ph | Me | Me | Me | H | H |
| CH₂-4-Br—Ph | Me | H | H | Me | Me |
| CH₂-4-Br—Ph | Et | Me | Me | H | H |
| CH₂-4-Br—Ph | Et | H | H | Me | Me |
| CH₂-4-Br—Ph | i-Pr | Me | Me | H | H |
| CH₂-4-Br—Ph | i-Pr | H | H | Me | Me |
| CH₂-4-Br—Ph | Pr | Me | Me | H | H |
| CH₂-4-Br—Ph | Pr | H | H | Me | Me |
| CH₂-4-Br—Ph | Bu | Me | Me | H | H |
| CH₂-4-Br—Ph | Bu | H | H | Me | Me |

TABLE L162

| R¹ | R² | R⁵ | R⁶ | R⁷ | R⁸ |
|---|---|---|---|---|---|
| CH₂-2-CF₃—Ph | H | Me | Me | H | H |
| CH₂-2-CF₃—Ph | H | H | H | Me | Me |
| CH₂-2-CF₃—Ph | Me | Me | Me | H | H |
| CH₂-2-CF₃—Ph | Me | H | H | Me | Me |
| CH₂-2-CF₃—Ph | Et | Me | Me | H | H |
| CH₂-2-CF₃—Ph | Et | H | H | Me | Me |
| CH₂-2-CF₃—Ph | i-Pr | Me | Me | H | H |
| CH₂-2-CF₃—Ph | i-Pr | H | H | Me | Me |
| CH₂-2-CF₃—Ph | Pr | Me | Me | H | H |
| CH₂-2-CF₃—Ph | Pr | H | H | Me | Me |
| CH₂-2-CF₃—Ph | Bu | Me | Me | H | H |
| CH₂-2-CF₃—Ph | Bu | H | H | Me | Me |
| CH₂-3-CF₃—Ph | H | Me | Me | H | H |
| CH₂-3-CF₃—Ph | H | H | H | Me | Me |
| CH₂-3-CF₃—Ph | Me | Me | Me | H | H |
| CH₂-3-CF₃—Ph | Me | H | H | Me | Me |
| CH₂-3-CF₃—Ph | Et | Me | Me | H | H |
| CH₂-3-CF₃—Ph | Et | H | H | Me | Me |
| CH₂-3-CF₃—Ph | i-Pr | Me | Me | H | H |
| CH₂-3-CF₃—Ph | i-Pr | H | H | Me | Me |
| CH₂-3-CF₃—Ph | Pr | Me | Me | H | H |
| CH₂-3-CF₃—Ph | Pr | H | H | Me | Me |
| CH₂-3-CF₃—Ph | Bu | Me | Me | H | H |
| CH₂-3-CF₃—Ph | Bu | H | H | Me | Me |

TABLE L163

| R¹ | R² | R⁵ | R⁶ | R⁷ | R⁸ |
|---|---|---|---|---|---|
| CH₂-4-CF₃—Ph | H | Me | Me | H | H |
| CH₂-4-CF₃—Ph | H | H | H | Me | Me |
| CH₂-4-CF₃—Ph | Me | Me | Me | H | H |
| CH₂-4-CF₃—Ph | Me | H | H | Me | Me |
| CH₂-4-CF₃—Ph | Et | Me | Me | H | H |
| CH₂-4-CF₃—Ph | Et | H | H | Me | Me |
| CH₂-4-CF₃—Ph | i-Pr | Me | Me | H | H |
| CH₂-4-CF₃—Ph | i-Pr | H | H | Me | Me |
| CH₂-4-CF₃—Ph | Pr | Me | Me | H | H |
| CH₂-4-CF₃—Ph | Pr | H | H | Me | Me |
| CH₂-4-CF₃—Ph | Bu | Me | Me | H | H |
| CH₂-4-CF₃—Ph | Bu | H | H | Me | Me |
| CH₂-2-OCF₃—Ph | H | Me | Me | H | H |
| CH₂-2-OCF₃—Ph | H | H | H | Me | Me |
| CH₂-2-OCF₃—Ph | Me | Me | Me | H | H |
| CH₂-2-OCF₃—Ph | Me | H | H | Me | Me |
| CH₂-2-OCF₃—Ph | Et | Me | Me | H | H |
| CH₂-2-OCF₃—Ph | Et | H | H | Me | Me |
| CH₂-2-OCF₃—Ph | i-Pr | Me | Me | H | H |
| CH₂-2-OCF₃—Ph | i-Pr | H | H | Me | Me |
| CH₂-2-OCF₃—Ph | Pr | Me | Me | H | H |
| CH₂-2-OCF₃—Ph | Pr | H | H | Me | Me |
| CH₂-2-OCF₃—Ph | Bu | Me | Me | H | H |
| CH₂-2-OCF₃—Ph | Bu | H | H | Me | Me |

TABLE L164

| R¹ | R² | R⁵ | R⁶ | R⁷ | R⁸ |
|---|---|---|---|---|---|
| CH₂-3-OCF₃—Ph | H | Me | Me | H | H |
| CH₂-3-OCF₃—Ph | H | H | H | Me | Me |
| CH₂-3-OCF₃—Ph | Me | Me | Me | H | H |
| CH₂-3-OCF₃—Ph | Me | H | H | Me | Me |
| CH₂-3-OCF₃—Ph | Et | Me | Me | H | H |
| CH₂-3-OCF₃—Ph | Et | H | H | Me | Me |
| CH₂-3-OCF₃—Ph | i-Pr | Me | Me | H | H |
| CH₂-3-OCF₃—Ph | i-Pr | H | H | Me | Me |
| CH₂-3-OCF₃—Ph | Pr | Me | Me | H | H |
| CH₂-3-OCF₃—Ph | Pr | H | H | Me | Me |
| CH₂-3-OCF₃—Ph | Bu | Me | Me | H | H |
| CH₂-3-OCF₃—Ph | Bu | H | H | Me | Me |
| CH₂-4-OCF₃—Ph | H | Me | Me | H | H |
| CH₂-4-OCF₃—Ph | H | H | H | Me | Me |
| CH₂-4-OCF₃—Ph | Me | Me | Me | H | H |
| CH₂-4-OCF₃—Ph | Me | H | H | Me | Me |
| CH₂-4-OCF₃—Ph | Et | Me | Me | H | H |
| CH₂-4-OCF₃—Ph | Et | H | H | Me | Me |
| CH₂-4-OCF₃—Ph | i-Pr | Me | Me | H | H |
| CH₂-4-OCF₃—Ph | i-Pr | H | H | Me | Me |
| CH₂-4-OCF₃—Ph | Pr | Me | Me | H | H |
| CH₂-4-OCF₃—Ph | Pr | H | H | Me | Me |
| CH₂-4-OCF₃—Ph | Bu | Me | Me | H | H |
| CH₂-4-OCF₃—Ph | Bu | H | H | Me | Me |

TABLE L165

| R¹ | R² | R⁵ | R⁶ | R⁷ | R⁸ |
|---|---|---|---|---|---|
| CH₂-2-Ph—Ph | H | Me | Me | H | H |
| CH₂-2-Ph—Ph | H | H | H | Me | Me |
| CH₂-2-Ph—Ph | Me | Me | Me | H | H |
| CH₂-2-Ph—Ph | Me | H | H | Me | Me |
| CH₂-2-Ph—Ph | Et | Me | Me | H | H |
| CH₂-2-Ph—Ph | Et | H | H | Me | Me |
| CH₂-2-Ph—Ph | i-Pr | Me | Me | H | H |
| CH₂-2-Ph—Ph | i-Pr | H | H | Me | Me |
| CH₂-2-Ph—Ph | Pr | Me | Me | H | H |
| CH₂-2-Ph—Ph | Pr | H | H | Me | Me |
| CH₂-2-Ph—Ph | Bu | Me | Me | H | H |
| CH₂-2-Ph—Ph | Bu | H | H | Me | Me |
| CH₂-3-Ph—Ph | H | Me | Me | H | H |
| CH₂-3-Ph—Ph | H | H | H | Me | Me |
| CH₂-3-Ph—Ph | Me | Me | Me | H | H |
| CH₂-3-Ph—Ph | Me | H | H | Me | Me |
| CH₂-3-Ph—Ph | Et | Me | Me | H | H |
| CH₂-3-Ph—Ph | Et | H | H | Me | Me |
| CH₂-3-Ph—Ph | i-Pr | Me | Me | H | H |
| CH₂-3-Ph—Ph | i-Pr | H | H | Me | Me |
| CH₂-3-Ph—Ph | Pr | Me | Me | H | H |
| CH₂-3-Ph—Ph | Pr | H | H | Me | Me |
| CH₂-3-Ph—Ph | Bu | Me | Me | H | H |
| CH₂-3-Ph—Ph | Bu | H | H | Me | Me |

TABLE L166

| R¹ | R² | R⁵ | R⁶ | R⁷ | R⁸ |
|---|---|---|---|---|---|
| CH₂-4-Ph—Ph | H | Me | Me | H | H |
| CH₂-4-Ph—Ph | H | H | H | Me | Me |
| CH₂-4-Ph—Ph | Me | Me | Me | H | H |
| CH₂-4-Ph—Ph | Me | H | H | Me | Me |
| CH₂-4-Ph—Ph | Et | Me | Me | H | H |
| CH₂-4-Ph—Ph | Et | H | H | Me | Me |
| CH₂-4-Ph—Ph | i-Pr | Me | Me | H | H |
| CH₂-4-Ph—Ph | i-Pr | H | H | Me | Me |
| CH₂-4-Ph—Ph | Pr | Me | Me | H | H |
| CH₂-4-Ph—Ph | Pr | H | H | Me | Me |
| CH₂-4-Ph—Ph | Bu | Me | Me | H | H |
| CH₂-4-Ph—Ph | Bu | H | H | Me | Me |
| CH₂-2-OPh—Ph | H | Me | Me | H | H |
| CH₂-2-OPh—Ph | H | H | H | Me | Me |
| CH₂-2-OPh—Ph | Me | Me | Me | H | H |
| CH₂-2-OPh—Ph | Me | H | H | Me | Me |
| CH₂-2-OPh—Ph | Et | Me | Me | H | H |
| CH₂-2-OPh—Ph | Et | H | H | Me | Me |
| CH₂-2-OPh—Ph | i-Pr | Me | Me | H | H |
| CH₂-2-OPh—Ph | i-Pr | H | H | Me | Me |
| CH₂-2-OPh—Ph | Pr | Me | Me | H | H |
| CH₂-2-OPh—Ph | Pr | H | H | Me | Me |
| CH₂-2-OPh—Ph | Bu | Me | Me | H | H |
| CH₂-2-OPh—Ph | Bu | H | H | Me | Me |

TABLE L167

| R¹ | R² | R⁵ | R⁶ | R⁷ | R⁸ |
|---|---|---|---|---|---|
| CH₂-3-OPh—Ph | H | Me | Me | H | H |
| CH₂-3-OPh—Ph | H | H | H | Me | Me |
| CH₂-3-OPh—Ph | Me | Me | Me | H | H |
| CH₂-3-OPh—Ph | Me | H | H | Me | Me |
| CH₂-3-OPh—Ph | Et | Me | Me | H | H |
| CH₂-3-OPh—Ph | Et | H | H | Me | Me |
| CH₂-3-OPh—Ph | i-Pr | Me | Me | H | H |
| CH₂-3-OPh—Ph | i-Pr | H | H | Me | Me |
| CH₂-3-OPh—Ph | Pr | Me | Me | H | H |
| CH₂-3-OPh—Ph | Pr | H | H | Me | Me |
| CH₂-3-OPh—Ph | Bu | Me | Me | H | H |
| CH₂-3-OPh—Ph | Bu | H | H | Me | Me |
| CH₂-4-OPh—Ph | H | Me | Me | H | H |
| CH₂-4-OPh—Ph | H | H | H | Me | Me |
| CH₂-4-OPh—Ph | Me | Me | Me | H | H |
| CH₂-4-OPh—Ph | Me | H | H | Me | Me |
| CH₂-4-OPh—Ph | Et | Me | Me | H | H |
| CH₂-4-OPh—Ph | Et | H | H | Me | Me |
| CH₂-4-OPh—Ph | i-Pr | Me | Me | H | H |
| CH₂-4-OPh—Ph | i-Pr | H | H | Me | Me |
| CH₂-4-OPh—Ph | Pr | Me | Me | H | H |
| CH₂-4-OPh—Ph | Pr | H | H | Me | Me |
| CH₂-4-OPh—Ph | Bu | Me | Me | H | H |
| CH₂-4-OPh—Ph | Bu | H | H | Me | Me |

TABLE L168

| R¹ | R² | R⁵ | R⁶ | R⁷ | R⁸ |
|---|---|---|---|---|---|
| CH₂-2-CN—Ph | H | Me | Me | H | H |
| CH₂-2-CN—Ph | H | H | H | Me | Me |
| CH₂-2-CN—Ph | Me | Me | Me | H | H |
| CH₂-2-CN—Ph | Me | H | H | Me | Me |
| CH₂-2-CN—Ph | Et | Me | Me | H | H |
| CH₂-2-CN—Ph | Et | H | H | Me | Me |
| CH₂-2-CN—Ph | i-Pr | Me | Me | H | H |
| CH₂-2-CN—Ph | i-Pr | H | H | Me | Me |
| CH₂-2-CN—Ph | Pr | Me | Me | H | H |
| CH₂-2-CN—Ph | Pr | H | H | Me | Me |
| CH₂-2-CN—Ph | Bu | Me | Me | H | H |
| CH₂-2-CN—Ph | Bu | H | H | Me | Me |
| CH₂-3-CN—Ph | H | Me | Me | H | H |
| CH₂-3-CN—Ph | H | H | H | Me | Me |
| CH₂-3-CN—Ph | Me | Me | Me | H | H |
| CH₂-3-CN—Ph | Me | H | H | Me | Me |
| CH₂-3-CN—Ph | Et | Me | Me | H | H |
| CH₂-3-CN—Ph | Et | H | H | Me | Me |
| CH₂-3-CN—Ph | i-Pr | Me | Me | H | H |
| CH₂-3-CN—Ph | i-Pr | H | H | Me | Me |
| CH₂-3-CN—Ph | Pr | Me | Me | H | H |
| CH₂-3-CN—Ph | Pr | H | H | Me | Me |
| CH₂-3-CN—Ph | Bu | Me | Me | H | H |
| CH₂-3-CN—Ph | Bu | H | H | Me | Me |

TABLE L169

| R¹ | R² | R⁵ | R⁶ | R⁷ | R⁸ |
|---|---|---|---|---|---|
| CH₂-4-CN—Ph | H | Me | Me | H | H |
| CH₂-4-CN—Ph | H | H | H | Me | Me |
| CH₂-4-CN—Ph | Me | Me | Me | H | H |
| CH₂-4-CN—Ph | Me | H | H | Me | Me |
| CH₂-4-CN—Ph | Et | Me | Me | H | H |
| CH₂-4-CN—Ph | Et | H | H | Me | Me |
| CH₂-4-CN—Ph | i-Pr | Me | Me | H | H |
| CH₂-4-CN—Ph | i-Pr | H | H | Me | Me |
| CH₂-4-CN—Ph | Pr | Me | Me | H | H |
| CH₂-4-CN—Ph | Pr | H | H | Me | Me |
| CH₂-4-CN—Ph | Bu | Me | Me | H | H |
| CH₂-4-CN—Ph | Bu | H | H | Me | Me |
| CH₂-2-NO₂—Ph | H | Me | Me | H | H |
| CH₂-2-NO₂—Ph | H | H | H | Me | Me |
| CH₂-2-NO₂—Ph | Me | Me | Me | H | H |
| CH₂-2-NO₂—Ph | Me | H | H | Me | Me |
| CH₂-2-NO₂—Ph | Et | Me | Me | H | H |
| CH₂-2-NO₂—Ph | Et | H | H | Me | Me |
| CH₂-2-NO₂—Ph | i-Pr | Me | Me | H | H |
| CH₂-2-NO₂—Ph | i-Pr | H | H | Me | Me |
| CH₂-2-NO₂—Ph | Pr | Me | Me | H | H |
| CH₂-2-NO₂—Ph | Pr | H | H | Me | Me |
| CH₂-2-NO₂—Ph | Bu | Me | Me | H | H |
| CH₂-2-NO₂—Ph | Bu | H | H | Me | Me |

TABLE L170

| R¹ | R² | R⁵ | R⁶ | R⁷ | R⁸ |
|---|---|---|---|---|---|
| CH₂-3-NO₂—Ph | H | Me | Me | H | H |
| CH₂-3-NO₂—Ph | H | H | H | Me | Me |
| CH₂-3-NO₂—Ph | Me | Me | Me | H | H |
| CH₂-3-NO₂—Ph | Me | H | H | Me | Me |
| CH₂-3-NO₂—Ph | Et | Me | Me | H | H |
| CH₂-3-NO₂—Ph | Et | H | H | Me | Me |
| CH₂-3-NO₂—Ph | i-Pr | Me | Me | H | H |
| CH₂-3-NO₂—Ph | i-Pr | H | H | Me | Me |
| CH₂-3-NO₂—Ph | Pr | Me | Me | H | H |
| CH₂-3-NO₂—Ph | Pr | H | H | Me | Me |
| CH₂-3-NO₂—Ph | Bu | Me | Me | H | H |
| CH₂-3-NO₂—Ph | Bu | H | H | Me | Me |
| CH₂-4-NO₂—Ph | H | Me | Me | H | H |
| CH₂-4-NO₂—Ph | H | H | H | Me | Me |
| CH₂-4-NO₂—Ph | Me | Me | Me | H | H |
| CH₂-4-NO₂—Ph | Me | H | H | Me | Me |
| CH₂-4-NO₂—Ph | Et | Me | Me | H | H |
| CH₂-4-NO₂—Ph | Et | H | H | Me | Me |
| CH₂-4-NO₂—Ph | i-Pr | Me | Me | H | H |
| CH₂-4-NO₂—Ph | i-Pr | H | H | Me | Me |
| CH₂-4-NO₂—Ph | Pr | Me | Me | H | H |
| CH₂-4-NO₂—Ph | Pr | H | H | Me | Me |
| CH₂-4-NO₂—Ph | Bu | Me | Me | H | H |
| CH₂-4-NO₂—Ph | Bu | H | H | Me | Me |

TABLE L171

| R¹ | R² | R⁵ | R⁶ | R⁷ | R⁸ |
|---|---|---|---|---|---|
| CH₂—Py2 | H | Me | Me | H | H |
| CH₂—Py2 | H | H | H | Me | Me |
| CH₂—Py2 | Me | Me | Me | H | H |
| CH₂—Py2 | Me | H | H | Me | Me |
| CH₂—Py2 | Et | Me | Me | H | H |
| CH₂—Py2 | Et | H | H | Me | Me |
| CH₂—Py2 | i-Pr | Me | Me | H | H |
| CH₂—Py2 | i-Pr | H | H | Me | Me |
| CH₂—Py2 | Pr | Me | Me | H | H |
| CH₂—Py2 | Pr | H | H | Me | Me |
| CH₂—Py2 | Bu | Me | Me | H | H |
| CH₂—Py2 | Bu | H | H | Me | Me |
| CH₂—Py3 | H | Me | Me | H | H |
| CH₂—Py3 | H | H | H | Me | Me |
| CH₂—Py3 | Me | Me | Me | H | H |
| CH₂—Py3 | Me | H | H | Me | Me |
| CH₂—Py3 | Et | Me | Me | H | H |
| CH₂—Py3 | Et | H | H | Me | Me |
| CH₂—Py3 | i-Pr | Me | Me | H | H |
| CH₂—Py3 | i-Pr | H | H | Me | Me |
| CH₂—Py3 | Pr | Me | Me | H | H |
| CH₂—Py3 | Pr | H | H | Me | Me |
| CH₂—Py3 | Bu | Me | Me | H | H |
| CH₂—Py3 | Bu | H | H | Me | Me |

TABLE L172

| R¹ | R² | R⁵ | R⁶ | R⁷ | R⁸ |
|---|---|---|---|---|---|
| CH₂—Py4 | H | Me | Me | H | H |
| CH₂—Py4 | H | H | H | Me | Me |
| CH₂—Py4 | Me | Me | Me | H | H |
| CH₂—Py4 | Me | H | H | Me | Me |
| CH₂—Py4 | Et | Me | Me | H | H |
| CH₂—Py4 | Et | H | H | Me | Me |
| CH₂—Py4 | i-Pr | Me | Me | H | H |
| CH₂—Py4 | i-Pr | H | H | Me | Me |
| CH₂—Py4 | Pr | Me | Me | H | H |
| CH₂—Py4 | Pr | H | H | Me | Me |
| CH₂—Py4 | Bu | Me | Me | H | H |
| CH₂—Py4 | Bu | H | H | Me | Me |
| CH₂-5-CF₃—Py2 | H | Me | Me | H | H |
| CH₂-5-CF₃—Py2 | H | H | H | Me | Me |
| CH₂-5-CF₃—Py2 | Me | Me | Me | H | H |
| CH₂-5-CF₃—Py2 | Me | H | H | Me | Me |
| CH₂-5-CF₃—Py2 | Et | Me | Me | H | H |
| CH₂-5-CF₃—Py2 | Et | H | H | Me | Me |
| CH₂-5-CF₃—Py2 | i-Pr | Me | Me | H | H |
| CH₂-5-CF₃—Py2 | i-Pr | H | H | Me | Me |
| CH₂-5-CF₃—Py2 | Pr | Me | Me | H | H |
| CH₂-5-CF₃—Py2 | Pr | H | H | Me | Me |
| CH₂-5-CF₃—Py2 | Bu | Me | Me | H | H |
| CH₂-5-CF₃—Py2 | Bu | H | H | Me | Me |

TABLE L173

| R¹ | R² | R⁵ | R⁶ | R⁷ | R⁸ |
|---|---|---|---|---|---|
| CH₂-6-CF₃—Py3 | H | Me | Me | H | H |
| CH₂-6-CF₃—Py3 | H | H | H | Me | Me |
| CH₂-6-CF₃—Py3 | Me | Me | Me | H | H |
| CH₂-6-CF₃—Py3 | Me | H | H | Me | Me |
| CH₂-6-CF₃—Py3 | Et | Me | Me | H | H |
| CH₂-6-CF₃—Py3 | Et | H | H | Me | Me |
| CH₂-6-CF₃—Py3 | i-Pr | Me | Me | H | H |
| CH₂-6-CF₃—Py3 | i-Pr | H | H | Me | Me |
| CH₂-6-CF₃—Py3 | Pr | Me | Me | H | H |
| CH₂-6-CF₃—Py3 | Pr | H | H | Me | Me |
| CH₂-6-CF₃—Py3 | Bu | Me | Me | H | H |
| CH₂-6-CF₃—Py3 | Bu | H | H | Me | Me |
| CH₂-furan-2-yl | H | Me | Me | H | H |
| CH₂-furan-2-yl | H | H | H | Me | Me |
| CH₂-furan-2-yl | Me | Me | Me | H | H |
| CH₂-furan-2-yl | Me | H | H | Me | Me |
| CH₂-furan-2-yl | Et | Me | Me | H | H |
| CH₂-furan-2-yl | Et | H | H | Me | Me |
| CH₂-furan-2-yl | i-Pr | Me | Me | H | H |
| CH₂-furan-2-yl | i-Pr | H | H | Me | Me |
| CH₂-furan-2-yl | Pr | Me | Me | H | H |
| CH₂-furan-2-yl | Pr | H | H | Me | Me |
| CH₂-furan-2-yl | Bu | Me | Me | H | H |
| CH₂-furan-2-yl | Bu | H | H | Me | Me |

TABLE L174

| R¹ | R² | R⁵ | R⁶ | R⁷ | R⁸ |
|---|---|---|---|---|---|
| CH₂-furan-3-yl | H | Me | Me | H | H |
| CH₂-furan-3-yl | H | H | H | Me | Me |
| CH₂-furan-3-yl | Me | Me | Me | H | H |
| CH₂-furan-3-yl | Me | H | H | Me | Me |
| CH₂-furan-3-yl | Et | Me | Me | H | H |
| CH₂-furan-3-yl | Et | H | H | Me | Me |
| CH₂-furan-3-yl | i-Pr | Me | Me | H | H |
| CH₂-furan-3-yl | i-Pr | H | H | Me | Me |
| CH₂-furan-3-yl | Pr | Me | Me | H | H |
| CH₂-furan-3-yl | Pr | H | H | Me | Me |
| CH₂-furan-3-yl | Bu | Me | Me | H | H |
| CH₂-furan-3-yl | Bu | H | H | Me | Me |
| CH₂-furan-3-yl | H | Me | Me | H | H |
| CH₂-5-CF₃-furan-2-yl | H | H | H | Me | Me |
| CH₂-5-CF₃-furan-2-yl | Me | Me | Me | H | H |
| CH₂-5-CF₃-furan-2-yl | Me | H | H | Me | Me |
| CH₂-5-CF₃-furan-2-yl | Et | Me | Me | H | H |
| CH₂-5-CF₃-furan-2-yl | Et | H | H | Me | Me |
| CH₂-5-CF₃-furan-2-yl | i-Pr | Me | Me | H | H |
| CH₂-5-CF₃-furan-2-yl | i-Pr | H | H | Me | Me |
| CH₂-5-CF₃-furan-2-yl | Pr | Me | Me | H | H |
| CH₂-5-CF₃-furan-2-yl | Pr | H | H | Me | Me |
| CH₂-5-CF₃-furan-2-yl | Bu | Me | Me | H | H |
| CH₂-5-CF₃-furan-2-yl | Bu | H | H | Me | Me |
| CH₂-5-CF₃-furan-2-yl | | | | | |

TABLE L175

| R¹ | R² | R⁵ | R⁶ | R⁷ | R⁸ |
|---|---|---|---|---|---|
| CH₂-5-CF₃-furan-3-yl | H | Me | Me | H | H |
| CH₂-5-CF₃-furan-3-yl | H | H | H | Me | Me |
| CH₂-5-CF₃-furan-3-yl | Me | Me | Me | H | H |
| CH₂-5-CF₃-furan-3-yl | Me | H | H | Me | Me |
| CH₂-5-CF₃-furan-3-yl | Et | Me | Me | H | H |
| CH₂-5-CF₃-furan-3-yl | Et | H | H | Me | Me |
| CH₂-5-CF₃-furan-3-yl | i-Pr | Me | Me | H | H |
| CH₂-5-CF₃-furan-3-yl | i-Pr | H | H | Me | Me |
| CH₂-5-CF₃-furan-3-yl | Pr | Me | Me | H | H |
| CH₂-5-CF₃-furan-3-yl | Pr | H | H | Me | Me |
| CH₂-5-CF₃-furan-3-yl | Bu | Me | Me | H | H |
| CH₂-5-CF₃-furan-3-yl | Bu | H | H | Me | Me |
| CH₂-thiophen-2-yl | H | Me | Me | H | H |
| CH₂-thiophen-2-yl | H | H | H | Me | Me |
| CH₂-thiophen-2-yl | Me | Me | Me | H | H |
| CH₂-thiophen-2-yl | Me | H | H | Me | Me |
| CH₂-thiophen-2-yl | Et | Me | Me | H | H |
| CH₂-thiophen-2-yl | Et | H | H | Me | Me |
| CH₂-thiophen-2-yl | i-Pr | Me | Me | H | H |
| CH₂-thiophen-2-yl | i-Pr | H | H | Me | Me |
| CH₂-thiophen-2-yl | Pr | Me | Me | H | H |
| CH₂-thiophen-2-yl | Pr | H | H | Me | Me |
| CH₂-thiophen-2-yl | Bu | Me | Me | H | H |
| CH₂-thiophen-2-yl | Bu | H | H | Me | Me |

TABLE L176

| R¹ | R² | R⁵ | R⁶ | R⁷ | R⁸ |
|---|---|---|---|---|---|
| CH₂-thiophen-3-yl | H | Me | Me | H | H |
| CH₂-thiophen-3-yl | H | H | H | Me | Me |
| CH₂-thiophen-3-yl | Me | Me | Me | H | H |
| CH₂-thiophen-3-yl | Me | H | H | Me | Me |
| CH₂-thiophen-3-yl | Et | Me | Me | H | H |
| CH₂-thiophen-3-yl | Et | H | H | Me | Me |
| CH₂-thiophen-3-yl | i-Pr | Me | Me | H | H |
| CH₂-thiophen-3-yl | i-Pr | H | H | Me | Me |
| CH₂-thiophen-3-yl | Pr | Me | Me | H | H |
| CH₂-thiophen-3-yl | Pr | H | H | Me | Me |
| CH₂-thiophen-3-yl | Bu | Me | Me | H | H |
| CH₂-thiophen-3-yl | Bu | H | H | Me | Me |
| CH₂-5-CF₃-thiophen-2-yl | H | Me | Me | H | H |
| CH₂-5-CF₃-thiophen-2-yl | H | H | H | Me | Me |
| CH₂-5-CF₃-thiophen-2-yl | Me | Me | Me | H | H |
| CH₂-5-CF₃-thiophen-2-yl | Me | H | H | Me | Me |
| CH₂-5-CF₃-thiophen-2-yl | Et | Me | Me | H | H |
| CH₂-5-CF₃-thiophen-2-yl | Et | H | H | Me | Me |
| CH₂-5-CF₃-thiophen-2-yl | i-Pr | Me | Me | H | H |
| CH₂-5-CF₃-thiophen-2-yl | i-Pr | H | H | Me | Me |
| CH₂-5-CF₃-thiophen-2-yl | Pr | Me | Me | H | H |
| CH₂-5-CF₃-thiophen-2-yl | Pr | H | H | Me | Me |
| CH₂-5-CF₃-thiophen-2-yl | Bu | Me | Me | H | H |
| CH₂-5-CF₃-thiophen-2-yl | Bu | H | H | Me | Me |

TABLE L177

| R¹ | R² | R⁵ | R⁶ | R⁷ | R⁸ |
|---|---|---|---|---|---|
| CH₂-5-CF₃-thiophen-3-yl | H | Me | Me | H | H |
| CH₂-5-CF₃-thiophen-3-yl | H | H | H | Me | Me |
| CH₂-5-CF₃-thiophen-3-yl | Me | Me | Me | H | H |
| CH₂-5-CF₃-thiophen-3-yl | Me | H | H | Me | Me |
| CH₂-5-CF₃-thiophen-3-yl | Et | Me | Me | H | H |
| CH₂-5-CF₃-thiophen-3-yl | Et | H | H | Me | Me |
| CH₂-5-CF₃-thiophen-3-yl | i-Pr | Me | Me | H | H |
| CH₂-5-CF₃-thiophen-3-yl | i-Pr | H | H | Me | Me |
| CH₂-5-CF₃-thiophen-3-yl | Pr | Me | Me | H | H |
| CH₂-5-CF₃-thiophen-3-yl | Pr | H | H | Me | Me |
| CH₂-5-CF₃-thiophen-3-yl | Bu | Me | Me | H | H |
| CH₂-5-CF₃-thiophen-3-yl | Bu | H | H | Me | Me |
| pyridazin-3-yl | H | Me | Me | H | H |
| pyridazin-3-yl | H | H | H | Me | Me |
| pyridazin-3-yl | Me | Me | Me | H | H |
| pyridazin-3-yl | Me | H | H | Me | Me |
| pyridazin-3-yl | Et | Me | Me | H | H |
| pyridazin-3-yl | Et | H | H | Me | Me |
| pyridazin-3-yl | i-Pr | Me | Me | H | H |
| pyridazin-3-yl | i-Pr | H | H | Me | Me |
| pyridazin-3-yl | Pr | Me | Me | H | H |
| pyridazin-3-yl | Pr | H | H | Me | Me |
| pyridazin-3-yl | Bu | Me | Me | H | H |
| pyridazin-3-yl | Bu | H | H | Me | Me |

TABLE L178

| R¹ | R² | R⁵ | R⁶ | R⁷ | R⁸ |
|---|---|---|---|---|---|
| pyridazin-4-yl | H | Me | Me | H | H |
| pyridazin-4-yl | H | H | H | Me | Me |
| pyridazin-4-yl | Me | Me | Me | H | H |
| pyridazin-4-yl | Me | H | H | Me | Me |
| pyridazin-4-yl | Et | Me | Me | H | H |
| pyridazin-4-yl | Et | H | H | Me | Me |
| pyridazin-4-yl | i-Pr | Me | Me | H | H |
| pyridazin-4-yl | i-Pr | H | H | Me | Me |
| pyridazin-4-yl | Pr | Me | Me | H | H |
| pyridazin-4-yl | Pr | H | H | Me | Me |
| pyridazin-4-yl | Bu | Me | Me | H | H |
| pyridazin-4-yl | Bu | H | H | Me | Me |
| pyrimidin-2-yl | H | Me | Me | H | H |
| pyrimidin-2-yl | H | H | H | Me | Me |
| pyrimidin-2-yl | Me | Me | Me | H | H |
| pyrimidin-2-yl | Me | H | H | Me | Me |
| pyrimidin-2-yl | Et | Me | Me | H | H |
| pyrimidin-2-yl | Et | H | H | Me | Me |
| pyrimidin-2-yl | i-Pr | Me | Me | H | H |
| pyrimidin-2-yl | i-Pr | H | H | Me | Me |
| pyrimidin-2-yl | Pr | Me | Me | H | H |
| pyrimidin-2-yl | Pr | H | H | Me | Me |
| pyrimidin-2-yl | Bu | Me | Me | H | H |
| pyrimidin-2-yl | Bu | H | H | Me | Me |

TABLE L179

| R¹ | R² | R⁵ | R⁶ | R⁷ | R⁸ |
|---|---|---|---|---|---|
| pyrimidin-4-yl | H | Me | Me | H | H |
| pyrimidin-4-yl | H | H | H | Me | Me |
| pyrimidin-4-yl | Me | Me | Me | H | H |
| pyrimidin-4-yl | Me | H | H | Me | Me |
| pyrimidin-4-yl | Et | Me | Me | H | H |
| pyrimidin-4-yl | Et | H | H | Me | Me |
| pyrimidin-4-yl | i-Pr | Me | Me | H | H |
| pyrimidin-4-yl | i-Pr | H | H | Me | Me |
| pyrimidin-4-yl | Pr | Me | Me | H | H |
| pyrimidin-4-yl | Pr | H | H | Me | Me |
| pyrimidin-4-yl | Bu | Me | Me | H | H |
| pyrimidin-4-yl | Bu | H | H | Me | Me |
| pyrimidin-5-yl | H | Me | Me | H | H |
| pyrimidin-5-yl | H | H | H | Me | Me |
| pyrimidin-5-yl | Me | Me | Me | H | H |
| pyrimidin-5-yl | Me | H | H | Me | Me |
| pyrimidin-5-yl | Et | Me | Me | H | H |
| pyrimidin-5-yl | Et | H | H | Me | Me |
| pyrimidin-5-yl | i-Pr | Me | Me | H | H |
| pyrimidin-5-yl | i-Pr | H | H | Me | Me |
| pyrimidin-5-yl | Pr | Me | Me | H | H |
| pyrimidin-5-yl | Pr | H | H | Me | Me |
| pyrimidin-5-yl | Bu | Me | Me | H | H |
| pyrimidin-5-yl | Bu | H | H | Me | Me |

TABLE L180

| R¹ | R² | R⁵ | R⁶ | R⁷ | R⁸ |
|---|---|---|---|---|---|
| pyrazin-2-yl | H | Me | Me | H | H |
| pyrazin-2-yl | H | H | H | Me | Me |
| pyrazin-2-yl | Me | Me | Me | H | H |
| pyrazin-2-yl | Me | H | H | Me | Me |
| pyrazin-2-yl | Et | Me | Me | H | H |
| pyrazin-2-yl | Et | H | H | Me | Me |
| pyrazin-2-yl | i-Pr | Me | Me | H | H |
| pyrazin-2-yl | i-Pr | H | H | Me | Me |
| pyrazin-2-yl | Pr | Me | Me | H | H |
| pyrazin-2-yl | Pr | H | H | Me | Me |
| pyrazin-2-yl | Bu | Me | Me | H | H |
| pyrazin-2-yl | Bu | H | H | Me | Me |

The compound (L-3), wherein R¹³ is a methyl group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1234).

The compound (L-3), wherein R¹³ is a hydrogen atom, R¹⁴ is a methyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1235).

The compound (L-3), wherein R¹³ is a hydrogen atom, R¹⁴ is a hydrogen atom, R¹⁵ is a methyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1236).

The compound (L-3), wherein R¹³ is a hydrogen atom, R¹⁴ is a hydrogen atom, R¹⁵ is a hydrogen atom, R¹⁶ is a methyl group, X is an oxygen atom, and the combination of R¹, R², R⁵, R⁶, R⁷, and R⁸ is any one of the combinations shown in [Table L100] to [Table L180] (hereinafter, referred to as a compound group SX1237).

The compound, R¹³ is a methyl group, R¹⁴ is a methyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1238).

The compound (L-3), wherein R¹³ is a methyl group, R¹⁴ is a hydrogen atom, R¹⁵ is a methyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1239).

The compound (L-3), wherein R¹³ is a methyl group, R¹⁴ is a hydrogen atom, R¹⁵ is a hydrogen atom, R¹⁶ is a methyl group, X is an oxygen atom, and the combination of R¹, R², R⁵, R⁶, R⁷, and R⁸ is any one of the combinations shown in [Table L100] to [Table L180] (hereinafter, referred to as a compound group SX1240).

The compound (L-3), wherein R¹³ is a hydrogen atom, R¹⁴ is a methyl group, R¹⁵ is a methyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1241).

The compound (L-3), wherein R¹³ is a hydrogen atom, R¹⁴ is a methyl group, R¹⁵ is a hydrogen atom, R¹⁶ is a methyl group, X is an oxygen atom, and the combination of R¹, R², R⁵, R⁶, R⁷, and R⁸ is any one of the combinations shown in [Table L100] to [Table L180] (hereinafter, referred to as a compound group SX1242).

The compound (L-3), wherein R¹³ is a hydrogen atom, R¹⁴ is a hydrogen atom, R¹⁵ is a methyl group, R¹⁶ is a methyl group, X is an oxygen atom, and the combination of R¹, R², R⁵, R⁶, R⁷, and R⁸ is any one of the combinations shown in [Table L100] to [Table L180] (hereinafter, referred to as a compound group SX1243).

The compound (L-3), wherein R¹³ is a methyl group, R¹⁴ is a methyl group, R¹⁵ is a methyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1244).

The compound (L-3), wherein R¹³ is a hydrogen atom, R¹⁴ is a methyl group, R¹⁵ is a methyl group, R¹⁶ is a methyl group, X is an oxygen atom, and the combination of R¹, R², R⁵, R⁶, R⁷, and R⁸ is any one of the combinations shown in [Table L100] to [Table L180] (hereinafter, referred to as a compound group SX1245).

The compound (L-3), wherein R¹³ is a fluorine atom, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1246).

The compound (L-3), wherein R¹³ is a hydrogen atom, R¹⁴ is a fluorine atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1247).

The compound (L-3), wherein R¹³ is a hydrogen atom, R¹⁴ is a hydrogen atom, R¹⁵ is a fluorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1248).

The compound (L-3), wherein R¹³ is a hydrogen atom, R¹⁴ is a hydrogen atom, R¹⁵ is a hydrogen atom, R¹⁶ is a fluorine atom, X is an oxygen atom, and the combination of R¹, R², R⁵, R⁶, R⁷, and R⁸ is any one of the combinations shown in [Table L100] to [Table L180] (hereinafter, referred to as a compound group SX1249).

The compound (L-3), wherein R¹³ is a chlorine atom, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1250).

The compound (L-3), wherein R¹³ is a hydrogen atom, R¹⁴ is a chlorine atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1251).

The compound (L-3), wherein R¹³ is a hydrogen atom, R¹⁴ is a hydrogen atom, R¹⁵ is a chlorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1252).

The compound (L-3), wherein R¹³ is a hydrogen atom, R¹⁴ is a hydrogen atom, R¹⁵ is a hydrogen atom, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1253).

The compound (L-3), wherein R¹³ is a bromine atom, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1254).

The compound (L-3), wherein R¹³ is a hydrogen atom, R¹⁴ is a bromine atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1255).

The compound (L-3), wherein R¹³ is a hydrogen atom, R¹⁴ is a hydrogen atom, R¹⁵ is a bromine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1256).

The compound (L-3), wherein R¹³ is a hydrogen atom, R¹⁴ is a hydrogen atom, R¹⁵ is a hydrogen atom, R¹⁶ is a bromine atom, X is an oxygen atom, and the combination of R¹, R², R⁵, R⁶, R⁷, and R⁸ is any one of the combinations shown in [Table L100] to [Table L180] (hereinafter, referred to as a compound group SX1257).

The compound (L-3), wherein R¹³ is an iodine atom, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1258).

The compound (L-3), wherein R¹³ is a hydrogen atom, R¹⁴ is an iodine atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1259).

The compound (L-3), wherein R¹³ is a hydrogen atom, R¹⁴ is a hydrogen atom, R¹⁵ is an iodine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1260).

The compound (L-3), wherein R¹³ is a hydrogen atom, R¹⁴ is a hydrogen atom, R¹⁵ is a hydrogen atom, R¹⁶ is an iodine atom, X is an oxygen atom, and the combination of R¹, R², R⁵, R⁶, R⁷, and R⁸ is any one of the combinations shown in [Table L100] to [Table L180] (hereinafter, referred to as a compound group SX1261).

The compound (L-3), wherein R¹³ is a methoxy group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1262).

The compound (L-3), wherein R¹³ is a hydrogen atom, R¹⁴ is a methoxy group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1263).

The compound (L-3), wherein R¹³ is a hydrogen atom, R¹⁴ is a hydrogen atom, R¹⁵ is a methoxy group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1264).

The compound (L-3), wherein R¹³ is a hydrogen atom, R¹⁴ is a hydrogen atom, R¹⁵ is a hydrogen atom, R¹⁶ is a methoxy group, X is an oxygen atom, and the combination of R¹, R², R⁵, R⁶, R⁷, and R⁸ is any one of the combinations shown in [Table L100] to [Table L180] (hereinafter, referred to as a compound group SX1265).

The compound (L-3), wherein R¹³ is a trifluoromethyl group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1266).

The compound (L-3), wherein R¹³ is a hydrogen atom, R¹⁴ is a trifluoromethyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1267).

The compound (L-3), wherein R¹³ is a hydrogen atom, R¹⁴ is a hydrogen atom, R¹⁵ is a trifluoromethyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1268).

The compound (L-3), wherein R¹³ is a hydrogen atom, R¹⁴ is a hydrogen atom, R¹⁵ is a hydrogen atom, R¹⁶ is a trifluoromethyl group, X is an oxygen atom, and the combination of R¹, R², R⁵, R⁶, R⁷, and R⁸ is any one of the combinations shown in [Table L100] to [Table L180] (hereinafter, referred to as a compound group SX1269).

The compound (L-3), wherein R¹³ is a cyclopropyl group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1270).

The compound (L-3), wherein R¹³ is a hydrogen atom, R¹⁴ is a cyclopropyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1271).

The compound (L-3), wherein R¹³ is a hydrogen atom, R¹⁴ is a hydrogen atom, R¹⁵ is a cyclopropyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1272).

The compound (L-3), wherein R¹³ is a hydrogen atom, R¹⁴ is a hydrogen atom, R¹⁵ is a hydrogen atom, R¹⁶ is a cyclopropyl group, X is an oxygen atom, and the combination of R¹, R², R⁵, R⁶, R⁷, and R⁸ is any one of the combinations shown in [Table L100] to [Table L180] (hereinafter, referred to as a compound group SX1273).

The compound (L-3), wherein R¹³ is a cyclobutyl group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations Shown in [Table L100] to [Table L180] (hereinafter, referred to as a compound group SX1274).

The compound (L-3), wherein R¹³ is a hydrogen atom, R¹⁴ is a cyclobutyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1275).

The compound (L-3), wherein R¹³ is a hydrogen atom, R¹⁴ is a hydrogen atom, R¹⁵ is a cyclobutyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1276).

The compound (L-3), wherein R¹³ is a hydrogen atom, R¹⁴ is a hydrogen atom, R¹⁵ is a hydrogen atom, R¹⁶ is a cyclobutyl group, X is an oxygen atom, and the combination of R¹, R², R⁵, R⁶, R⁷, and R⁸ is any one of the combinations shown in [Table L100] to [Table L180] (hereinafter, referred to as a compound group SX1277).

The compound (L-3), wherein R¹³ is a cyclopentyl group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1278).

The compound (L-3), wherein R¹³ is a hydrogen atom, R¹⁴ is a cyclopentyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1279).

The compound (L-3), wherein R¹³ is a hydrogen atom, R¹⁴ is a hydrogen atom, R¹⁵ is a cyclopentyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1280).

The compound (L-3), wherein R¹³ is a hydrogen atom, R¹⁴ is a hydrogen atom, R¹⁵ is a hydrogen atom, R¹⁶ is a cyclopentyl group, X is an oxygen atom, and the combination of R¹, R², R⁵, R⁶, R⁷, and R⁸ is any one of the combinations shown in [Table L100] to [Table L180] (hereinafter, referred to as a compound group SX1281).

The compound (L-3), wherein R¹³ is a cyclohexyl group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1282).

The compound (L-3), wherein R¹³ is a hydrogen atom, R¹⁴ is a cyclohexyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1283).

The compound (L-3), wherein R¹³ is a hydrogen atom, R¹⁴ is a hydrogen atom, R¹⁵ is a cyclohexyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1284).

The compound (L-3), wherein R¹³ is a hydrogen atom, R¹⁴ is a hydrogen atom, R¹⁵ is a hydrogen atom, R¹⁶ is a cyclohexyl group, X is an oxygen atom, and the combination of R¹, R², R⁵, R⁶, R⁷, and R⁸ is any one of the combinations shown in [Table L100] to [Table L180] (hereinafter, referred to as a compound group SX1285).

The compound (L-3), wherein R¹³ is a phenyl group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1286).

The compound (L-3), wherein R¹³ is a hydrogen atom, R¹⁴ is a phenyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1287).

The compound (L-3), wherein R¹³ is a hydrogen atom, R¹⁴ is a hydrogen atom, R¹⁵ is a phenyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1288).

The compound (L-3), wherein R¹³ is a hydrogen atom, R¹⁴ is a hydrogen atom, R¹⁵ is a hydrogen atom, R¹⁶ is a phenyl group, X is an oxygen atom, and the combination of R¹, R², R⁵, R⁶, R⁷, and R⁸ is any one of the combinations shown in [Table L100] to [Table L180] (hereinafter, referred to as a compound group SX1289).

The compound (L-3), wherein R¹³ is an acetyl group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1290).

The compound (L-3), wherein R¹³ is a hydrogen atom, R¹⁴ is an acetyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1291).

The compound (L-3), wherein R¹³ is a hydrogen atom, R¹⁴ is a hydrogen atom, R¹⁵ is an acetyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1292).

The compound (L-3), wherein R¹³ is a hydrogen atom, R¹⁴ is a hydrogen atom, R¹⁵ is a hydrogen atom, R¹⁶ is an acetyl group, X is an oxygen atom, and the combination of R¹, R², R⁵, R⁶, R⁷, and R⁸ is any one of the combinations shown in [Table L100] to [Table L180] (hereinafter, referred to as a compound group SX1293).

The compound (L-3), wherein R¹³ is a trifluoroacetyl group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1294).

The compound (L-3), wherein R¹³ is a hydrogen atom, R¹⁴ is a trifluoroacetyl group, R¹⁵ is a hydrogen atom, Rie is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1295).

The compound (L-3), wherein R¹³ is a hydrogen atom, R¹⁴ is a hydrogen atom, R¹⁶ is a trifluoroacetyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1296).

The compound (L-3), wherein R¹³ is a hydrogen atom, R¹⁴ is a hydrogen atom, R¹⁵ is a hydrogen atom, R¹⁶ is a trifluoroacetyl group, X is an oxygen atom, and the combination of R¹, R², R⁵, R⁶, R⁷, and R⁸ is any one of the combinations shown in [Table L100] to [Table L180] (hereinafter, referred to as a compound group SX1297).

The compound (L-3), wherein R¹³ is a methoxycarbonyl group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1298).

The compound (L-3), wherein R¹³ is a hydrogen atom, R¹⁴ is a methoxycarbonyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1299).

The compound (L-3), wherein R¹³ is a hydrogen atom, R¹⁴ is a hydrogen atom, R¹⁵ is a methoxycarbonyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1300).

The compound (L-3), wherein R¹³ is a hydrogen atom, R¹⁴ is a hydrogen atom, R¹⁵ is a hydrogen atom, R¹⁶ is a methoxycarbonyl group, X is an oxygen atom, and the combination of R¹, R², R⁵, R⁶, R⁷, and R⁸ is any one of the combinations shown in [Table L100] to [Table L180] (hereinafter, referred to as a compound group SX1301).

The compound (L-3), wherein R¹³ is a trifluoromethoxycarbonyl group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1302).

The compound (L-3), wherein R¹³ is a hydrogen atom, R¹⁴ is a trifluoromethoxycarbonyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1303).

The compound (L-3), wherein R¹³ is a hydrogen atom, R¹⁴ is a hydrogen atom, R¹⁵ is a trifluoromethoxycarbonyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1304).

The compound (L-3), wherein R¹³ is a hydrogen atom, R¹⁴ is a hydrogen atom, R¹⁵ is a hydrogen atom, R¹⁶ is a trifluoromethoxycarbonyl group, X is an oxygen atom, and the combination of R¹, R², R⁵, R⁶, R⁷, and R⁸ is any one of the combinations shown in [Table L100] to [Table L180] (hereinafter, referred to as a compound group SX1305).

The compound (L-3), wherein R¹³ is an acetoxy group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1306).

The compound (L-3), wherein R¹³ is a hydrogen atom, R¹⁴ is an acetoxy group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1307).

The compound (L-3), wherein R¹³ is a hydrogen atom, R¹⁴ is a hydrogen atom, R¹⁵ is an acetoxy group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1308).

The compound (L-3), wherein R¹³ is a hydrogen atom, R¹⁴ is a hydrogen atom, R¹⁵ is a hydrogen atom, R¹⁶ is an acetoxy group, X is an oxygen atom, and the combination of R¹, R², R⁵, R⁶, R⁷, and R⁸ is any one of the combinations shown in [Table L100] to [Table L180] (hereinafter, referred to as a compound group SX1309).

The compound (L-3), wherein R¹³ is a trifluoroacetoxy group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1310).

The compound (L-3), wherein R¹³ is a hydrogen atom, R¹⁴ is a trifluoroacetoxy group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1311).

The compound (L-3), wherein R¹³ is a hydrogen atom, R¹⁴ is a hydrogen atom, R¹⁵ is a trifluoroacetoxy group, R¹⁶ is a hydrogen atom, X is an oxygen atom, and the combination of R, R², R⁵, R⁶, R⁷, and R⁸ is any one of the combinations shown in [Table L100] to [Table L180] (hereinafter, referred to as a compound group SX1312).

The compound (L-3), wherein R¹³ is a hydrogen atom, R¹⁴ is a hydrogen atom, R¹⁵ is a hydrogen atom, R¹⁶ is a trifluoroacetoxy group, X is an oxygen atom, and the Combination of R¹, R², R⁵, R⁶, R⁷, and R⁸ is any one of the combinations shown in [Table L100] to [Table L180] (hereinafter, referred to as a compound group SX1313).

The compound (L-3), wherein R¹³ is a cyano group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1314).

The compound (L-3), wherein R¹³ is a hydrogen atom, R¹⁴ is a cyano group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1315).

The compound (L-3), wherein R¹³ is a hydrogen atom, R¹⁴ is a hydrogen atom, R¹⁵ is a cyano group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1316).

The compound (L-3), wherein R¹³ is a hydrogen atom, R¹⁴ is a hydrogen atom, R¹⁵ is a hydrogen atom, R¹⁶ is a cyano group, X is an oxygen atom, and the combination of R¹, R², R⁵, R⁶, R⁷, and R⁸ is any one of the combinations shown in [Table L100] to [Table L180] (hereinafter, referred to as a compound group SX1317).

The compound (L-3), wherein R¹³ is a formyl group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1318).

The compound (L-3), wherein R¹³ is a hydrogen atom, R¹⁴ is a formyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1319).

The compound (L-3), wherein R¹³ is a hydrogen atom, R¹⁴ is a hydrogen atom, R¹⁵ is a formyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1320).

The compound (L-3), wherein R¹³ is a hydrogen atom, R¹⁴ is a hydrogen atom, R¹⁵ is a hydrogen atom, R¹⁶ is a formyl group, X is an oxygen atom, and the combination of R¹, R², R⁵, R⁶, R⁷, and R⁸ is any one of the combinations shown in [Table L100] to [Table L180] (hereinafter, referred to as a compound group SX1321).

The compound (L-3), wherein R¹³ is a carboxy group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1322).

The compound (L-3), wherein R¹³ is a hydrogen atom, R¹⁴ is a carboxy group, R¹⁵ is a hydrogen atom, R¹⁶ is a hydrogen atom, X is an oxygen atom, and the combination of shown in [Table L100] to [Table L180] (hereinafter, referred to as a compound group SX1323).

The compound (L-3), wherein R¹³ is a hydrogen atom, R¹⁴ is a hydrogen atom, R¹⁵ is a carboxy group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1324).

The compound (L-3), wherein R¹³ is a hydrogen atom, R¹⁴ is a hydrogen atom, R¹⁵ is a hydrogen atom, R¹⁶ is a carboxy group, X is an oxygen atom, and the combination of R¹, R², R⁵, R⁶, R⁷, and R⁸ is any one of the combinations shown in [Table L100] to [Table L180] (hereinafter, referred to as a compound group SX1325).

The compound (L-3), wherein R¹³ is a nitro group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1326).

The compound (L-3), wherein R¹³ is a hydrogen atom, R¹⁴ is a nitro group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1327).

The compound (L-3), wherein R¹³ is a hydrogen atom, R¹⁴ is a hydrogen atom, R¹⁵ is a nitro group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1328).

The compound (L-3), wherein R¹³ is a hydrogen atom, R¹⁴ is a hydrogen atom, R¹⁵ is a hydrogen atom, R¹⁶ is a nitro group, X is an oxygen atom, and the combination of R¹, R², R⁵, R⁶, R⁷, and R⁸ is any one of the combinations shown in [Table L100] to [Table L180] (hereinafter, referred to as a compound group SX1329).

The compound (L-3), wherein R¹³ is a hydroxy group, R¹⁴ is a hydrogen atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1330).

The compound (L-3), wherein R¹³ is a hydrogen atom, R¹⁴ is a hydroxy group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1331).

The compound (L-3), wherein R¹³ is a hydrogen atom, R¹⁴ is a hydrogen atom, R¹⁵ is a hydroxy group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1332).

The compound (L-3), wherein R¹³ is a hydrogen atom, R¹⁴ is a hydrogen atom, R¹⁵ is a hydrogen atom, R¹⁶ is a hydroxy group, X is an oxygen atom, and the combination of R¹, R², R⁵, R⁶, R⁷, and R⁸ is any one of the combinations shown in [Table L100] to [Table L180] (hereinafter, referred to as a compound group SX1333).

The compound (L-3), wherein R¹³ is a fluorine atom, R¹⁴ is a fluorine atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1334).

The compound (L-3), wherein R¹³ is a fluorine atom, R¹⁴ is a hydrogen atom, R¹⁵ is a fluorine atom, R¹⁶ is a hydrogen atom, X is an oxygen atom, and the combination of R¹, R², R³, R⁶, R⁷, and R⁸ is any one of the combinations shown in [Table L100] to [Table L180] (hereinafter, referred to as a compound group SX1335).

The compound (L-3), wherein R¹³ is a fluorine atom, R¹⁴ is a hydrogen atom, R¹⁵ is a hydrogen atom, R¹⁶ is a fluorine atom, X is an oxygen atom, and the combination of R¹, R², R⁵, R⁶, R⁷, and R⁸ is any one of the combinations shown in [Table L100] to [Table L180] (hereinafter, referred to as a compound group SX1336).

The compound (L-3), wherein R¹³ is a hydrogen atom, R¹⁴ is a fluorine atom, R¹⁵ is a fluorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1337).

The compound (L-3), wherein R¹³ is a hydrogen atom, R¹⁴ is a fluorine atom, R¹⁵ is a hydrogen atom, R¹⁶ is a fluorine atom, X is an oxygen atom, and the combination of R¹, R², R⁵, R⁶, R⁷, and R⁸ is any one of the combinations Shown in [Table L100] to [Table L180] (hereinafter, referred to as a compound group SX1338).

The compound (L-3), wherein R¹³ is a hydrogen atom, R¹⁴ is a hydrogen atom, R¹⁵ is a fluorine atom, R¹⁶ is a fluorine atom, X is an oxygen atom, and the combination of R¹, R², R⁵, R⁶, R⁷, and R⁸ is any one of the combinations shown in [Table L100] to [Table L180] (hereinafter, referred to as a compound group SX1339).

The compound (L-3), wherein R¹³ is a fluorine atom, R¹⁴ is a fluorine atom, R¹⁵ is a fluorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1340).

The compound (L-3), wherein R¹³ is a hydrogen atom, R¹⁴ is a fluorine atom, R¹⁵ is a fluorine atom, R¹⁶ is a fluorine atom, X is an oxygen atom, and the combination of R¹, R², R⁵, R⁶, R⁷, and R⁸ is any one of the combinations shown in [Table L100] to [Table L180] (hereinafter, referred to as a compound group SX1341).

The compound (L-3), wherein R¹³ is a chlorine atom, R¹⁴ is a chlorine atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1342).

The compound (L-3), wherein R¹³ is a chlorine atom, R¹⁴ is a hydrogen atom, R¹⁵ is a chlorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1343).

The compound (L-3), wherein R¹³ is a chlorine atom, R¹⁴ is a hydrogen atom, R¹⁵ is a hydrogen atom, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1344).

The compound (L-3), wherein R¹³ is a hydrogen atom, R¹⁴ is a chlorine atom, R¹⁵ is a chlorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1345).

The compound (L-3), wherein R¹³ is a hydrogen atom, R¹⁴ is a chlorine atom, R¹⁵ is a hydrogen atom, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1346).

The compound (L-3), wherein R¹³ is a hydrogen atom, R¹⁴ is a hydrogen atom, R¹⁵ is a chlorine atom, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1347).

The compound (L-3), wherein R¹³ is a chlorine atom, R¹⁴ is a chlorine atom, R¹⁵ is a chlorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1348).

The compound (L-3), wherein R¹³ is a hydrogen atom, R¹⁴ is a chlorine atom, R¹⁵ is a chlorine atom, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1349).

The compound (L-3), wherein R¹³ is a bromine atom, R¹⁴ is a bromine atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1350).

The compound (L-3), wherein R¹³ is a bromine atom, R¹⁴ is a hydrogen atom, R¹⁵ is a bromine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1351).

The compound (L-3), wherein R¹³ is a bromine atom, R¹⁴ is a hydrogen atom, R¹⁵ is a hydrogen atom, R¹⁶ is a bromine atom, X is an oxygen atom, and the combination of R¹, R², R⁵, R⁶, R⁷, and R⁸ is any one of the combinations shown in [Table L100] to [Table L180] (hereinafter, referred to as a compound group SX1352).

The compound (L-3), wherein R¹³ is a hydrogen atom, R¹⁴ is a bromine atom, R¹⁵ is a bromine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1353).

The compound (L-3), wherein R¹³ is a hydrogen atom, R¹⁴ is a bromine atom, R¹⁵ is a hydrogen atom, R¹⁶ is a bromine atom, X is an oxygen atom, and the combination of R¹, R², R⁵, R⁶, R⁷, and R⁸ is any one of the combinations shown in [Table L100] to [Table L180] (hereinafter, referred to as a compound group SX1354).

The compound (L-3), wherein R¹³ is a hydrogen atom, R¹⁴ is a hydrogen atom, R¹⁵ is a bromine atom, R¹⁶ is a bromine atom, X is an oxygen atom, and the combination of R¹, R², R⁵, R⁶, R⁷, and R⁸ is any one of the combinations shown in [Table L100] to [Table L180] (hereinafter, referred to as a compound group SX1355).

The compound (L-3), wherein R¹³ is a bromine atom, R¹⁴ is a bromine atom, R¹⁵ is a bromine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1356).

The compound (L-3), wherein R¹³ is a hydrogen atom, R¹⁴ is a bromine atom, R¹⁵ is a bromine atom, R¹⁶ is a bromine atom, X is an oxygen atom, and the combination of R¹, R², R⁵, R⁶, R⁷, and R⁸ is any one of the combinations shown in [Table L100] to [Table L180] (hereinafter, referred to as a compound group SX1357).

The compound (L-3), wherein R¹³ is an iodine atom, R¹⁴ is an iodine atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1358).

The compound (L-3), wherein R¹³ is an iodine atom, R¹⁴ is a hydrogen atom, R¹⁵ is an iodine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1359).

The compound (L-3), wherein R¹³ is an iodine atom, R¹⁴ is a hydrogen atom, R¹⁵ is a hydrogen atom, R¹⁶ is an iodine atom, X is an oxygen atom, and the combination of R¹, R², R⁵, R⁶, R⁷, and R⁸ is any one of the combinations shown in [Table L100] to [Table L180] (hereinafter, referred to as a compound group SX1360).

The compound (L-3), wherein R¹³ is a hydrogen atom, R¹⁴ is an iodine atom, R¹⁵ is an iodine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1361).

The compound (L-3), wherein R¹³ is a hydrogen atom, R¹⁴ is an iodine atom, R¹⁵ is a hydrogen atom, R¹⁶ is an iodine atom, X is an oxygen atom, and the combination of R¹, R², R⁵, R⁶, R⁷, and R⁸ is any one of the combinations shown in [Table L100] to [Table L180] (hereinafter, referred to as a compound group SX1362).

The compound (L-3), wherein R¹³ is a hydrogen atom, R¹⁴ is a hydrogen atom, R¹⁵ is an iodine atom, R¹⁶ is an iodine atom, X is an oxygen atom, and the combination of R¹, R², R⁵, R⁶, R⁷, and R⁸ is any one of the combinations shown in [Table L100] to [Table L180] (hereinafter, referred to as a compound group SX1363).

The compound (L-3), wherein R¹³ is an iodine atom, R¹⁴ is an iodine atom, R¹⁵ is an iodine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1364).

The compound (L-3), wherein R¹³ is a hydrogen atom, R¹⁴ is an iodine atom, R¹⁵ is an iodine atom, R¹⁶ is an iodine atom, X is an oxygen atom, and the combination of R¹, R², R⁵, R⁶, R⁷, and R⁸ is any one of the combinations shown in [Table L100] to [Table L180] (hereinafter, referred to as a compound group SX1365).

The compound (L-3), wherein R¹³ is a fluorine atom, R¹⁴ is a chlorine atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1366).

The compound (L-3), wherein R¹³ is a fluorine atom, R¹⁴ is a hydrogen atom, R¹⁵ is a chlorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1367).

The compound (L-3), wherein R¹³ is a fluorine atom, R¹⁴ is a hydrogen atom, R¹⁵ is a hydrogen atom, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1368).

The compound (L-3), wherein R¹³ is a chlorine atom, R¹⁴ is a fluorine atom, R¹⁵ is a hydrogen atom, R¹⁶ is a hydrogen atom, X is an oxygen atom, and the combination of iodine atom, X is an oxygen atom, and the combination of R¹, R², R⁵, R⁶, R⁷, and R⁸ is any one of the combinations shown in [Table L100] to [Table L180] (hereinafter, referred to as a compound group SX1369).

The compound (L-3), wherein R¹³ is a chlorine atom, R¹⁴ is a hydrogen atom, R¹⁵ is a fluorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1370).

The compound (L-3), wherein R¹³ is a chlorine atom, R¹⁴ is a hydrogen atom, R¹⁵ is a hydrogen atom, R¹⁶ is a fluorine atom, X is an oxygen atom, and the combination of R¹, R², R⁵, R⁶, R⁷, and R⁸ is any one of the combinations shown in [Table L100] to [Table L180] (hereinafter, referred to as a compound group SX1371).

The compound (L-3), wherein R¹³ is a hydrogen atom, R¹⁴ is a fluorine atom, R¹⁵ is a chlorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1372).

The compound (L-3), wherein R¹³ is a hydrogen atom, R¹⁴ is a fluorine atom, R¹⁵ is a hydrogen atom, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1373).

The compound (L-3), wherein R¹³ is a hydrogen atom, R¹⁴ is a chlorine atom, R¹⁵ is a fluorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1374).

The compound (L-3), wherein R¹³ is a hydrogen atom, R¹⁴ is a chlorine atom, R¹⁵ is a hydrogen atom, R¹⁶ is a fluorine atom, X is an oxygen atom, and the combination of R¹, R², R⁵, R⁶, R⁷, and R⁸ is any one of the combinations shown in [Table L100] to [Table L180] (hereinafter, referred to as a compound group SX1375).

The compound (L-3), wherein R¹³ is a hydrogen atom, R¹⁴ is a hydrogen atom, R¹⁵ is a fluorine atom, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1376).

The compound (L-3), wherein R¹³ is a hydrogen atom, R¹⁴ is a hydrogen atom, R¹⁵ is a chlorine atom, R¹⁶ is a fluorine atom, X is an oxygen atom, and the combination of R¹, R², R⁵, R⁶, R⁷, and R⁸ is any one of the combinations shown in [Table L100] to [Table L180] (hereinafter, referred to as a compound group SX1377).

The compound (L-3), wherein R¹³ is a fluorine atom, R¹⁴ is a methyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1378).

The compound (L-3), wherein R¹³ is a fluorine atom, R¹⁴ is a hydrogen atom, R¹⁵ is a methyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1379).

The compound (L-3), wherein R¹³ is a fluorine atom, R¹⁴ is a hydrogen atom, R¹⁵ is a hydrogen atom, R¹⁶ is a methyl group, X is an oxygen atom, and the combination of R¹, R², R⁵, R⁶, R⁷, and R⁸ is any one of the combinations shown in [Table L100] to [Table L180] (hereinafter, referred to as a compound group SX1380).

The compound (L-3), wherein R¹³ is a methyl group, R¹⁴ is a fluorine atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1381).

The compound (L-3), wherein R¹³ is a methyl group, R¹⁴ is a hydrogen atom, R¹⁵ is a fluorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1382).

The compound (L-3), wherein R¹³ is a methyl group, R¹⁴ is a hydrogen atom, R¹⁵ is a hydrogen atom, R¹⁶ is a fluorine atom, X is an oxygen atom, and the combination of R¹, R², R⁵, R⁶, R⁷, and R⁸ is any one of the combinations shown in [Table L100] to [Table L180] (hereinafter, referred to as a compound group SX1383).

The compound (L-3), wherein R¹³ is a hydrogen atom, R¹⁴ is a fluorine atom, R¹⁵ is a methyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1384).

The compound (L-3), wherein R¹³ is a hydrogen atom, R¹⁴ is a fluorine atom, R¹⁵ is a hydrogen atom, R¹⁶ is a methyl group, X is an oxygen atom, and the combination of R¹, R², R³, R⁶, R⁷, and R⁸ is any one of the combinations shown in [Table L100] to [Table L180] (hereinafter, referred to as a compound group SX1385).

The compound (L-3), wherein R¹³ is a hydrogen atom, R¹⁴ is a methyl group, R¹⁵ is a fluorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1386).

The compound (L-3), wherein R¹³ is a hydrogen atom, R¹⁴ is a methyl group, R¹⁵ is a hydrogen atom, R¹⁶ is a fluorine atom, X is an oxygen atom, and the combination of R¹, R², R⁵, R⁶, R⁷, and R⁸ is any one of the combinations shown in [Table L100] to [Table L180] (hereinafter, referred to as a compound group SX1387).

The compound (L-3), wherein R¹³ is a hydrogen atom, R¹⁴ is a hydrogen atom, R¹⁵ is a fluorine atom, R¹⁶ is a methyl group, X is an oxygen atom, and the combination of R¹, R², R⁵, R⁶, R⁷, and R⁸ is any one of the combinations shown in [Table L100] to [Table L180] (hereinafter, referred to as a compound group SX1388).

The compound (L-3), wherein R¹³ is a hydrogen atom, R¹⁴ is a hydrogen atom, R¹⁶ is a methyl group, R¹⁶ is a fluorine atom, X is an oxygen atom, and the combination of R¹, R², R⁵, R⁶, R⁷, and R⁸ is any one of the combinations shown in [Table L100] to [Table L180] (hereinafter, referred to as a compound group SX1389).

The compound (L-3), wherein R¹³ is a fluorine atom, R¹⁴ is a methoxy group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1390).

The compound (L-3), wherein R¹³ is a fluorine atom, R¹⁴ is a hydrogen atom, R¹⁵ is a methoxy group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1391).

The compound (L-3), wherein R¹³ is a fluorine atom, R¹⁴ is a hydrogen atom, R¹⁵ is a hydrogen atom, R¹⁶ is a methoxy group, X is an oxygen atom, and the combination of R¹, R², R⁵, R⁶, R⁷, and R⁸ is any one of the combinations shown in [Table L100] to [Table L180] (hereinafter, referred to as a compound group SX1392).

The compound (L-3), wherein R¹³ is a methoxy group, R¹⁴ is a fluorine atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1393).

The compound (L-3), wherein R¹³ is a methoxy group, R¹⁴ is a hydrogen atom, R¹⁵ is a fluorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1394).

The compound (L-3), wherein R¹³ is a methoxy group, R¹⁴ is a hydrogen atom, R¹⁵ is a hydrogen atom, R¹⁶ is a fluorine atom, X is an oxygen atom, and the combination of R¹, R², R⁵, R⁶, R⁷, and R⁸ is any one of the combinations shown in [Table L100] to [Table L180] (hereinafter, referred to as a compound group SX1395).

The compound (L-3), wherein R¹³ is a hydrogen atom, R¹⁴ is a fluorine atom, R¹⁶ is a methoxy group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1396).

The compound (L-3), wherein R¹³ is a hydrogen atom, R¹⁴ is a fluorine atom, R¹⁵ is a hydrogen atom, R¹⁶ is a methoxy group, X is an oxygen atom, and the combination of R¹, R², R⁵, R⁶, R⁷, and R⁸ is any one of the combinations shown in [Table L100] to [Table L180] (hereinafter, referred to as a compound group SX1397).

The compound (L-3), wherein R¹³ is a hydrogen atom, R¹⁴ is a methoxy group, R¹⁵ is a fluorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1398).

The compound (L-3), wherein R¹³ is a hydrogen atom, R¹⁴ is a methoxy group, R¹⁵ is a hydrogen atom, R¹⁶ is a fluorine atom, X is an oxygen atom, and the combination of R¹, R², R⁵, R⁶, R⁷, and R⁸ is any one of the combinations shown in [Table L100] to [Table L180] (hereinafter, referred to as a compound group SX1399).

The compound (L-3), wherein R¹³ is a hydrogen atom, R¹⁴ is a hydrogen atom, R¹⁵ is a fluorine atom, R¹⁶ is a methoxy group, X is an oxygen atom, and the combination of R¹, R², R⁵, R⁶, R⁷, and R⁸ is any one of the combinations shown in [Table L100] to [Table L180] (hereinafter, referred to as a compound group SX1400).

The compound (L-3), wherein R¹³ is a hydrogen atom, R¹⁴ is a hydrogen atom, R¹⁵ is a methoxy group, R¹⁶ is a fluorine atom, X is an oxygen atom, and the combination of R¹, R², R⁵, R⁶, R⁷, and R⁸ is any one of the combinations shown in [Table L100] to [Table L180] (hereinafter, referred to as a compound group SX1401).

The compound (L-3), wherein R¹³ is a chlorine atom, R¹⁴ is a methyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1402).

The compound (L-3), wherein R¹³ is a chlorine atom, R¹⁴ is a hydrogen atom, R¹⁵ is a methyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1403).

The compound (L-3), wherein R¹³ is a chlorine atom, R¹⁴ is a hydrogen atom, R¹⁵ is a hydrogen atom, R¹⁶ is a methyl group, X is an oxygen atom, and the combination of R¹, R², R⁵, R⁶, R⁷, and R⁸ is any one of the combinations shown in [Table L100] to [Table L180] (hereinafter, referred to as a compound group SX1404).

The compound (L-3), wherein R¹³ is a methyl group, R¹⁴ is a chlorine atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1405).

The compound (L-3), wherein R¹³ is a methyl group, R¹⁴ is a hydrogen atom, R¹⁵ is a chlorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1406).

The compound (L-3), wherein R¹³ is a methyl group, R¹⁴ is a hydrogen atom, R¹⁵ is a hydrogen atom, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1407).

The compound (L-3), wherein R¹³ is a hydrogen atom, R¹⁴ is a chlorine atom, R¹⁵ is a methyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1408).

The compound (L-3), wherein R¹³ is a hydrogen atom, R¹⁴ is a chlorine atom, R¹⁵ is a hydrogen atom, R¹⁶ is a methyl group, X is an oxygen atom, and the combination of R¹, R², R⁵, R⁶, R⁷, and R⁸ is any one of the combinations shown in [Table L100] to [Table L180] (hereinafter, referred to as a compound group SX1409).

The compound (L-3), wherein R¹³ is a hydrogen atom, R¹⁴ is a methyl group, R¹⁵ is a chlorine atom, R¹⁶ is a hydrogen atom, X is an oxygen atom, and the combination of R¹, R², R³, R⁶, R⁷, and R⁸ is any one of the combinations shown in [Table L100] to [Table L180] (hereinafter, referred to as a compound group SX1410).

The compound (L-3), wherein R¹³ is a hydrogen atom, R¹⁴ is a methyl group, R¹⁵ is a hydrogen atom, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1411).

The compound (L-3), wherein R¹³ is a hydrogen atom, R¹⁴ is a hydrogen atom, R¹⁵ is a chlorine atom, R¹⁶ is a methyl group, X is an oxygen atom, and the combination of R¹, R², R⁵, R⁶, R⁷, and R⁸ is any one of the combinations shown in [Table L100] to [Table L180] (hereinafter, referred to as a compound group SX1412).

The compound (L-3), wherein R¹³ is a hydrogen atom, R¹⁴ is a hydrogen atom, R¹⁵ is a methyl group, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1413).

The compound (L-3), wherein R¹³ is a chlorine atom, R¹⁴ is a methoxy group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1414).

The compound (L-3), wherein R¹³ is a chlorine atom, R¹⁴ is a hydrogen atom, R¹⁵ is a methoxy group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1415).

The compound (L-3), wherein R¹³ is a chlorine atom, R¹⁴ is a hydrogen atom, R¹⁵ is a hydrogen atom, R¹⁶ is a methoxy group, X is an oxygen atom, and the combination of R¹, R², R⁵, R⁶, R⁷, and R⁸ is any one of the combinations shown in [Table L100] to [Table L180] (hereinafter, referred to as a compound group SX1416).

The compound (L-3), wherein R¹³ is a methoxy group, R¹⁴ is a chlorine atom, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1417).

The compound (L-3), wherein R¹³ is a methoxy group, R¹⁴ is a hydrogen atom, R¹⁵ is a chlorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1418).

The compound (L-3), wherein R¹³ is a methoxy group, R¹⁴ is a hydrogen atom, R¹⁵ is a hydrogen atom, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1419).

The compound (L-3), wherein R¹³ is a hydrogen atom, R¹⁴ is a chlorine atom, R¹⁵ is a methoxy group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1420).

The compound (L-3), wherein R¹³ is a hydrogen atom, R¹⁴ is a chlorine atom, R¹⁵ is a hydrogen atom, R¹⁶ is a methoxy group, X is an oxygen atom, and the combination of R¹, R², R³, R⁶, R⁷, and R⁸ is any one of the combinations shown in [Table L100] to [Table L180] (hereinafter, referred to as a compound group SX1421).

The compound (L-3), wherein R¹³ is a hydrogen atom, R¹⁴ is a methoxy group, R¹⁵ is a chlorine atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1422).

The compound (L-3), wherein R¹³ is a hydrogen atom, R¹⁴ is a methoxy group, R¹⁵ is a hydrogen atom, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1423).

The compound (L-3), wherein R¹³ is a hydrogen atom, R¹⁴ is a hydrogen atom, R¹⁵ is a chlorine atom, R¹⁶ is a methoxy group, X is an oxygen atom, and the combination of R¹, R², R⁵, R⁶, R⁷, and R⁸ is any one of the combinations shown in [Table L100] to [Table L180] (hereinafter, referred to as a compound group SX1424).

The compound (L-3), wherein R¹³ is a hydrogen atom, R¹⁴ is a hydrogen atom, R¹⁵ is a methoxy group, R¹⁶ is a chlorine atom, X is an oxygen atom, and the combination of R¹, R², R⁵, R⁶, R⁷, and R⁸ is any one of the combinations shown in [Table L100] to [Table L180] (hereinafter, referred to as a compound group SX1425).

The compound (L-3), wherein R¹³ is a methyl group, R¹⁴ is a methoxy group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1426).

The compound (L-3), wherein R¹³ is a methyl group, R¹⁴ is a hydrogen atom, R¹⁵ is a methoxy group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1427).

The compound (L-3), wherein R¹³ is a methyl group, R¹⁴ is a hydrogen atom, R¹⁵ is a hydrogen atom, R¹⁶ is a methoxy group, X is an oxygen atom, and the combination of R¹, R², R⁵, R⁶, R⁷, and R⁸ is any one of the combinations shown in [Table L100] to [Table L180] (hereinafter, referred to as a compound group SX1428).

The compound (L-3), wherein R¹³ is a methoxy group, R¹⁴ is a methyl group, R¹⁵ is a hydrogen atom, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1429).

The compound (L-3), wherein R¹³ is a methoxy group, R¹⁴ is a hydrogen atom, R¹⁵ is a methyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1430).

The compound (L-3), wherein R¹³ is a methoxy group, R¹⁴ is a hydrogen atom, R¹⁵ is a hydrogen atom, R¹⁶ is a methyl group, X is an oxygen atom, and the combination of R¹, R², R⁵, R⁶, R⁷, and R⁸ is any one of the combinations shown in [Table L100] to [Table L180] (hereinafter, referred to as a compound group SX1431).

The compound (L-3), wherein R¹³ is a hydrogen atom, R¹⁴ is a methyl group, R¹⁵ is a methoxy group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1432).

The compound (L-3), wherein R¹³ is a hydrogen atom, R¹⁴ is a methyl group, R¹⁵ is a hydrogen atom, R¹⁶ is a methoxy group, X is an oxygen atom, and the combination of R¹, R², R⁵, R⁶, R⁷, and R⁸ is any one of the combinations shown in [Table L100] to [Table L180] (hereinafter, referred to as a compound group SX1433).

The compound (L-3), wherein R¹³ is a hydrogen atom, R¹⁴ is a methoxy group, R¹⁵ is a methyl group, R¹⁶ is a hydrogen atom, X is an oxygen atom, and the combination of R¹, R², R⁵, R⁶, R⁷, and R⁸ is any one of the combinations shown in [Table L100] to [Table L180] (hereinafter, referred to as a compound group SX1434).

The compound (L-3), wherein R¹³ is a hydrogen atom, R¹⁴ is a methoxy group, R¹⁵ is a hydrogen atom, R¹⁶ is a methyl group, X is an oxygen atom, and the combination of R¹, R², R³, R⁶, R⁷, and R⁸ is any one of the combinations shown in [Table L100] to [Table L180] (hereinafter, referred to as a compound group SX1435).

The compound (L-3), wherein R¹³ is a hydrogen atom, R¹⁴ is a hydrogen atom, R¹⁵ is a methyl group, R¹⁶ is a methoxy group, X is an oxygen atom, and the combination of R¹, R², R⁵, R⁶, R⁷, and R⁸ is any one of the combinations shown in [Table L100] to [Table L180] (hereinafter, referred to as a compound group SX1436).

The compound (L-3), wherein R¹³ is a hydrogen atom, R¹⁴ is a hydrogen atom, R¹⁵ is a methoxy group, R¹⁶ is a methyl group, X is an oxygen atom, and the combination of R¹, R², R⁵, R⁶, R⁷, and R⁸ is any one of the combinations shown in [Table L100] to [Table L180] (hereinafter, referred to as a compound group SX1437).

The compound (L-3), wherein R¹³ is a hydrogen atom, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1438).

The compound (L-3), wherein R¹³ is a methyl group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1439).

The compound (L-3), wherein R¹³ is a hydrogen atom, R¹⁴ is a methyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1440).

The compound (L-3), wherein R¹³ is a hydrogen atom, R¹⁴ is a hydrogen atom, R¹⁵ is a methyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1441).

The compound (L-3), wherein R¹³ is a hydrogen atom, R¹⁴ is a hydrogen atom, R¹⁵ is a hydrogen atom, R¹⁶ is a methyl group, X is a sulfur atom, and the combination of R¹, R², R⁵, R⁶, R⁷, and R⁸ is any one of the combinations shown in [Table L100] to [Table L180] (hereinafter, referred to as a compound group SX1442).

The compound (L-3), wherein R¹³ is a methyl group, R¹⁴ is a methyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1443).

The compound (L-3), wherein R¹³ is a methyl group, R¹⁴ is a hydrogen atom, R¹⁵ is a methyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1444).

The compound (L-3), wherein R¹³ is a methyl group, R¹⁴ is a hydrogen atom, R¹⁵ is a hydrogen atom, R¹⁶ is a methyl group, X is a sulfur atom, and the combination of R¹, R², R⁵, R⁶, R⁷, and R⁸ is any one of the combinations shown in [Table L100] to [Table L180] (hereinafter, referred to as a compound group SX1445).

The compound (L-3), wherein R¹³ is a hydrogen atom, R¹⁴ is a methyl group, R¹⁵ is a methyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1446).

The compound (L-3), wherein R¹³ is a hydrogen atom, R¹⁴ is a methyl group, R¹⁵ is a hydrogen atom, R¹⁶ is a methyl group, X is a sulfur atom, and the combination of R¹, R², R⁵, R⁶, R⁷, and R⁸ is any one of the combinations shown in [Table L100] to [Table L180] (hereinafter, referred to as a compound group SX1447).

The compound (L-3), wherein R¹³ is a hydrogen atom, R¹⁴ is a hydrogen atom, R¹⁵ is a methyl group, R¹⁶ is a methyl group, X is a sulfur atom, and the combination of R¹, R², R⁵, R⁶, R⁷, and R⁸ is any one of the combinations shown in [Table L100] to [Table L180] (hereinafter, referred to as a compound group SX1448).

The compound (L-3), wherein R¹³ is a methyl group, R¹⁴ is a methyl group, R¹⁵ is a methyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1449).

The compound (L-3), wherein R¹³ is a hydrogen atom, R¹⁴ is a methyl group, R¹⁵ is a methyl group, R¹⁶ is a methyl group, X is a sulfur atom, and the combination of R¹, R², R⁵, R⁶, R⁷, and R⁸ is any one of the combinations shown in [Table L100] to [Table L180] (hereinafter, referred to as a compound group SX1450).

The compound (L-3), wherein R¹³ is a fluorine atom, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1451).

The compound (L-3), wherein R¹³ is a hydrogen atom, R¹⁴ is a fluorine atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1452).

The compound (L-3), wherein R¹³ is a hydrogen atom, R¹⁴ is a hydrogen atom, R¹⁵ is a fluorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1453).

The compound (L-3), wherein R¹³ is a hydrogen atom, R¹⁴ is a hydrogen atom, R¹⁵ is a hydrogen atom, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1454).

The compound (L-3), wherein R¹³ is a chlorine atom, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1455).

The compound (L-3), wherein R¹³ is a hydrogen atom, R¹⁴ is a chlorine atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1456).

The compound (L-3), wherein R¹³ is a hydrogen atom, R¹⁴ is a hydrogen atom, R¹⁵ is a chlorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1457).

The compound (L-3), wherein R¹³ is a hydrogen atom, R¹⁴ is a hydrogen atom, R¹⁵ is a hydrogen atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1458).

The compound (L-3), wherein R¹³ is a bromine atom, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1459).

The compound (L-3), wherein R¹³ is a hydrogen atom, R¹⁴ is a bromine atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1460).

The compound (L-3), wherein R¹³ is a hydrogen atom, R¹⁴ is a hydrogen atom, R¹⁵ is a bromine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1461).

The compound (L-3), wherein R¹³ is a hydrogen atom, R¹⁴ is a hydrogen atom, R¹⁵ is a hydrogen atom, R¹⁶ is a bromine atom, X is a sulfur atom, and the combination of R¹, R², R⁵, R⁶, R⁷, and R⁸ is any one of the combinations shown in [Table L100] to [Table L180] (hereinafter, referred to as a compound group SX1462).

The compound (L-3), wherein R¹³ is an iodine atom, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1463).

The compound (L-3), wherein R¹³ is a hydrogen atom, R¹⁴ is an iodine atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1464).

The compound (L-3), wherein R¹³ is a hydrogen atom, R¹⁴ is a hydrogen atom, R¹⁵ is an iodine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1465).

The compound (L-3), wherein R¹³ is a hydrogen atom, R¹⁴ is a hydrogen atom, R¹⁵ is a hydrogen atom, R¹⁶ is an iodine atom, X is a sulfur atom, and the combination of R¹, R², R⁵, R⁶, R⁷, and R⁸ is any one of the combinations shown in [Table L100] to [Table L180] (hereinafter, referred to as a compound group SX1466).

The compound (L-3), wherein R¹³ is a methoxy group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1467).

The compound (L-3), wherein R¹³ is a hydrogen atom, R¹⁴ is a methoxy group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1468).

The compound (L-3), wherein R¹³ is a hydrogen atom, R¹⁴ is a hydrogen atom, R¹⁵ is a methoxy group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1469).

The compound (L-3), wherein R¹³ is a hydrogen atom, R¹⁴ is a hydrogen atom, R¹⁵ is a hydrogen atom, R¹⁶ is a methoxy group, X is a sulfur atom, and the combination of R¹, R², R⁵, R⁶, R⁷, and R⁸ is any one of the combinations shown in [Table L100] to [Table L180] (hereinafter, referred to as a compound group SX1470).

The compound (L-3), wherein R¹³ is a trifluoromethyl group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1471).

The compound (L-3), wherein R¹³ is a hydrogen atom, R¹⁴ is a trifluoromethyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1472).

The compound (L-3), wherein R¹³ is a hydrogen atom, R¹⁴ is a hydrogen atom, R¹⁵ is a trifluoromethyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1473).

The compound (L-3), wherein R¹³ is a hydrogen atom, R¹⁴ is a hydrogen atom, R¹⁵ is a hydrogen atom, R¹⁶ is a trifluoromethyl group, X is a sulfur atom, and the Combination of R¹, R², R⁵, R⁶, R⁷, and R⁸ is any one of the combinations shown in [Table L100] to [Table L180] (hereinafter, referred to as a compound group SX1474).

The compound (L-3), wherein R¹³ is a cyclopropyl group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1475).

The compound (L-3), wherein R¹³ is a hydrogen atom, R¹⁴ is a cyclopropyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1476).

The compound (L-3), wherein R¹³ is a hydrogen atom, R¹⁴ is a hydrogen atom, R¹⁵ is a cyclopropyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1477).

The compound (L-3), wherein R¹³ is a hydrogen atom, R¹⁴ is a hydrogen atom, R¹⁵ is a hydrogen atom, R¹⁶ is a cyclopropyl group, X is a sulfur atom, and the combination of R¹, R², R⁵, R⁶, R⁷, and R⁸ is any one of the combinations shown in [Table L100] to [Table L180] (hereinafter, referred to as a compound group SX1478).

The compound (L-3), wherein R¹³ is a cyclobutyl group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1479).

The compound (L-3), wherein R¹³ is a hydrogen atom, R¹⁴ is a cyclobutyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1480).

The compound (L-3), wherein R¹³ is a hydrogen atom, R¹⁴ is a hydrogen atom, R¹⁵ is a cyclobutyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1481).

The compound (L-3), wherein R¹³ is a hydrogen atom, R¹⁴ is a hydrogen atom, R¹⁵ is a hydrogen atom, R¹⁶ is a cyclobutyl group, X is a sulfur atom, and the combination of R¹, R², R⁵, R⁶, R⁷, and R⁸ is any one of the combinations shown in [Table L100] to [Table L180] (hereinafter, referred to as a compound group SX1482).

The compound (L-3), wherein R¹³ is a cyclopentyl group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1483).

The compound (L-3), wherein R¹³ is a hydrogen atom, R¹⁴ is a cyclopentyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1484).

The compound (L-3), wherein R¹³ is a hydrogen atom, R¹⁴ is a hydrogen atom, R¹⁵ is a cyclopentyl group, R¹⁶ is a hydrogen atom, X is a sulfur atom, and the combination of R¹, R², R³, R⁶, R⁷, and R⁸ is any one of the combinations shown in [Table L100] to [Table L180] (hereinafter, referred to as a compound group SX1485).

The compound (L-3), wherein R¹³ is a hydrogen atom, R¹⁴ is a hydrogen atom, R¹⁵ is a hydrogen atom, R¹⁶ is a cyclopentyl group, X is a sulfur atom, and the combination of R¹, R², R⁵, R⁶, R⁷, and R⁸ is any one of the combinations shown in [Table L100] to [Table L180] (hereinafter, referred to as a compound group SX1486).

The compound (L-3), wherein R¹³ is a cyclohexyl group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1487).

The compound (L-3), wherein R¹³ is a hydrogen atom, R¹⁴ is a cyclohexyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1488).

The compound (L-3), wherein R¹³ is a hydrogen atom, R¹⁴ is a hydrogen atom, R¹⁵ is a cyclohexyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1489).

The compound (L-3), wherein R¹³ is a hydrogen atom, R¹⁴ is a hydrogen atom, R¹⁵ is a hydrogen atom, R¹⁶ is a cyclohexyl group, X is a sulfur atom, and the combination of R¹, R², R⁵, R⁶, R⁷, and R⁸ is any one of the combinations shown in [Table L100] to [Table L180] (hereinafter, referred to as a compound group SX1490).

The compound (L-3), wherein R¹³ is a phenyl group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1491).

The compound (L-3), wherein R¹³ is a hydrogen atom, R¹⁴ is a phenyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1492).

The compound (L-3), wherein R¹³ is a hydrogen atom, R¹⁴ is a hydrogen atom, R¹⁵ is a phenyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1493).

The compound (L-3), wherein R¹³ is a hydrogen atom, R¹⁴ is a hydrogen atom, R¹⁵ is a hydrogen atom, R¹⁶ is a phenyl group, X is a sulfur atom, and the combination of R¹, R², R⁵, R⁶, R⁷, and R⁸ is any one of the combinations shown in [Table L100] to [Table L180] (hereinafter, referred to as a compound group SX1494).

The compound (L-3), wherein R¹³ is an acetyl group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1495).

The compound (L-3), wherein R¹³ is a hydrogen atom, R¹⁴ is an acetyl group, R¹⁶ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1496).

The compound (L-3), wherein R¹³ is a hydrogen atom, R¹⁴ is a hydrogen atom, R¹⁵ is an acetyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1497).

The compound (L-3), wherein R¹³ is a hydrogen atom, R¹⁴ is a hydrogen atom, R¹⁵ is a hydrogen atom, R¹⁶ is an acetyl group, X is a sulfur atom, and the combination of R¹, R², R⁵, R⁶, R⁷, and R⁸ is any one of the combinations shown in [Table L100] to [Table L180] (hereinafter, referred to as a compound group SX1498).

The compound (L-3), wherein R¹³ is a trifluoroacetyl group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1499).

The compound (L-3), wherein R¹³ is a hydrogen atom, R¹⁴ is a trifluoroacetyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1500).

The compound (L-3), wherein R¹³ is a hydrogen atom, R¹⁴ is a hydrogen atom, R¹⁵ is a trifluoroacetyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1501).

The compound (L-3), wherein R¹³ is a hydrogen atom, R¹⁴ is a hydrogen atom, R¹⁵ is a hydrogen atom, R¹⁶ is a trifluoroacetyl group, X is a sulfur atom, and the combination of R¹, R², R⁵, R⁶, R⁷, and R⁸ is any one of the combinations shown in [Table L100] to [Table L180] (hereinafter, referred to as a compound group SX1502).

The compound (L-3), wherein R¹³ is a methoxycarbonyl group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1503).

The compound (L-3), wherein R¹³ is a hydrogen atom, R¹⁴ is a methoxycarbonyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1504).

The compound (L-3), wherein R¹³ is a hydrogen atom, R¹⁴ is a hydrogen atom, R¹⁵ is a methoxycarbonyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1505).

The compound (L-3), wherein R¹³ is a hydrogen atom, R¹⁴ is a hydrogen atom, R¹⁵ is a hydrogen atom, R¹⁶ is a methoxycarbonyl group, X is a sulfur atom, and the combination of R¹, R², R⁵, R⁶, R⁷, and R⁸ is any one of the combinations shown in [Table L100] to [Table L180] (hereinafter, referred to as a compound group SX1506).

The compound (L-3), wherein R¹³ is a trifluoromethoxycarbonyl group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1507).

The compound (L-3), wherein R¹³ is a hydrogen atom, R¹⁴ is a trifluoromethoxycarbonyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1508).

The compound (L-3), wherein R¹³ is a hydrogen atom, R¹⁴ is a hydrogen atom, R¹⁵ is a trifluoromethoxycarbonyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1509).

The compound (L-3), wherein R¹³ is a hydrogen atom, R¹⁴ is a hydrogen atom, R¹⁵ is a hydrogen atom, R¹⁶ is a trifluoromethoxycarbonyl group, X is a sulfur atom, and the combination of R¹, R², R⁵, R⁶, R⁷, and R⁸ is any one of the combinations shown in [Table L100] to [Table L180] (hereinafter, referred to as a compound group SX1510).

The compound (L-3), wherein R¹³ is an acetoxy group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1511).

The compound (L-3), wherein R¹³ is a hydrogen atom, R¹⁴ is an acetoxy group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1512).

The compound (L-3), wherein R¹³ is a hydrogen atom, R¹⁴ is a hydrogen atom, R¹⁵ is an acetoxy group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1513).

The compound (L-3), wherein R¹³ is a hydrogen atom, R¹⁴ is a hydrogen atom, R¹⁵ is a hydrogen atom, R¹⁶ is an acetoxy group, X is a sulfur atom, and the combination of R¹, R², R⁵, R⁶, R⁷, and R⁸ is any one of the combinations shown in [Table L100] to [Table L180] (hereinafter, referred to as a compound group SX1514).

The compound (L-3), wherein R¹³ is a trifluoroacetoxy group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1515).

The compound (L-3), wherein R¹³ is a hydrogen atom, R¹⁴ is a trifluoroacetoxy group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1516).

The compound (L-3), wherein R¹³ is a hydrogen atom, R¹⁴ is a hydrogen atom, R¹⁵ is a trifluoroacetoxy group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1517).

The compound (L-3), wherein R¹³ is a hydrogen atom, R¹⁴ is a hydrogen atom, R¹⁵ is a hydrogen atom, R¹⁶ is a trifluoroacetoxy group, X is a sulfur atom, and the combination of R¹, R², R⁵, R⁶, R⁷, and R⁸ is any one of the combinations shown in [Table L100] to [Table L180] (hereinafter, referred to as a compound group SX1518).

The compound (L-3), wherein R¹³ is a cyano group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1519).

The compound (L-3), wherein R¹³ is a hydrogen atom, R¹⁴ is a cyano group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1520).

The compound (L-3), wherein R¹³ is a hydrogen atom, R¹⁴ is a hydrogen atom, R¹⁵ is a cyano group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1521).

The compound (L-3), wherein R¹³ is a hydrogen atom, R¹⁴ is a hydrogen atom, R¹⁵ is a hydrogen atom, R¹⁶ is a cyano group, X is a sulfur atom, and the combination of R¹, R², R⁵, R⁶, R⁷, and R⁸ is any one of the combinations shown in [Table L100] to [Table L180] (hereinafter, referred to as a compound group SX1522).

The compound (L-3), wherein R¹³ is a formyl group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1523).

The compound (L-3), wherein R¹³ is a hydrogen atom, R¹⁴ is a formyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1524).

The compound (L-3), wherein R¹³ is a hydrogen atom, R¹⁴ is a hydrogen atom, R¹⁵ is a formyl group, R¹⁶ is a hydrogen atom, X is a sulfur atom, and the combination of R¹, R², R³, R⁶, R⁷, and R⁸ is any one of the combinations shown in [Table L100] to [Table L180] (hereinafter, referred to as a compound group SX1525).

The compound (L-3), wherein R¹³ is a hydrogen atom, R¹⁴ is a hydrogen atom, R¹⁵ is a hydrogen atom, R¹⁶ is a formyl group, X is a sulfur atom, and the combination of R¹, R², R⁵, R⁶, R⁷, and R⁸ is any one of the combinations shown in [Table L100] to [Table L180] (hereinafter, referred to as a compound group SX1526).

The compound (L-3), wherein R¹³ is a carboxy group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1527).

The compound (L-3), wherein R¹³ is a hydrogen atom, R¹⁴ is a carboxy group, R¹⁵ is a hydrogen atom, R¹⁶ is a hydrogen atom, X is a sulfur atom, and the combination of R¹, R², R⁵, R⁶, R⁷, and R⁸ is any one of the combinations Shown in [Table L100] to [Table L180] (hereinafter, referred to as a compound group SX1528).

The compound (L-3), wherein R¹³ is a hydrogen atom, R¹⁴ is a hydrogen atom, R¹⁵ is a carboxy group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1529).

The compound (L-3), wherein R¹³ is a hydrogen atom, R¹⁴ is a hydrogen atom, R¹⁵ is a hydrogen atom, R¹⁶ is a carboxy group, X is a sulfur atom, and the combination of R¹, R², R⁵, R⁶, R⁷, and R⁸ is any one of the combinations shown in [Table L100] to [Table L180] (hereinafter, referred to as a compound group SX1530).

The compound (L-3), wherein R¹³ is a nitro group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1531).

The compound (L-3), wherein R¹³ is a hydrogen atom, R¹⁴ is a nitro group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1532).

The compound (L-3), wherein R¹³ is a hydrogen atom, R¹⁴ is a hydrogen atom, R¹⁵ is a nitro group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1533).

The compound (L-3), wherein R¹³ is a hydrogen atom, R¹⁴ is a hydrogen atom, R¹⁵ is a hydrogen atom, R¹⁶ is a nitro group, X is a sulfur atom, and the combination of R¹, R², R⁵, R⁶, R⁷, and R⁸ is any one of the combinations shown in [Table L100] to [Table L180] (hereinafter, referred to as a compound group SX1534).

The compound (L-3), wherein R¹³ is a hydroxy group, R¹⁴ is a hydrogen atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1535).

The compound (L-3), wherein R¹³ is a hydrogen atom, R¹⁴ is a hydroxy group, Rib is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1536).

The compound (L-3), wherein R¹³ is a hydrogen atom, R¹⁴ is a hydrogen atom, R¹⁵ is a hydroxy group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1537).

The compound (L-3), wherein R¹³ is a hydrogen atom, R¹⁴ is a hydrogen atom, R¹⁵ is a hydrogen atom, R¹⁶ is a hydroxy group, X is a sulfur atom, and the combination of R¹, R², R⁵, R⁶, R⁷, and R⁸ is any one of the combinations shown in [Table L100] to [Table L180] (hereinafter, referred to as a compound group SX1538).

The compound (L-3), wherein R¹³ is a fluorine atom, R¹⁴ is a fluorine atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1539).

The compound (L-3), wherein R¹³ is a fluorine atom, R¹⁴ is a hydrogen atom, R¹⁵ is a fluorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1540).

The compound (L-3), wherein R¹³ is a fluorine atom, R¹⁴ is a hydrogen atom, R¹⁵ is a hydrogen atom, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1541).

The compound (L-3), wherein R¹³ is a hydrogen atom, R¹⁴ is a fluorine atom, R¹⁵ is a fluorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1542).

The compound (L-3), wherein R¹³ is a hydrogen atom, R¹⁴ is a fluorine atom, R¹⁵ is a hydrogen atom, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1543).

The compound (L-3), wherein R¹³ is a hydrogen atom, R¹⁴ is a hydrogen atom, R¹⁵ is a fluorine atom, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1544).

The compound (L-3), wherein R¹³ is a fluorine atom, R¹⁴ is a fluorine atom, R¹⁵ is a fluorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1545).

The compound (L-3), wherein R¹³ is a hydrogen atom, R¹⁴ is a fluorine atom, R¹⁵ is a fluorine atom, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1546).

The compound (L-3), wherein R¹³ is a chlorine atom, R¹⁴ is a chlorine atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1547).

The compound (L-3), wherein R¹³ is a chlorine atom, R¹⁴ is a hydrogen atom, R¹⁵ is a chlorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1548).

The compound (L-3), wherein R¹³ is a chlorine atom, R¹⁴ is a hydrogen atom, R¹⁵ is a hydrogen atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1549).

The compound (L-3), wherein R¹³ is a hydrogen atom, R¹⁴ is a chlorine atom, R¹⁵ is a chlorine atom, R¹⁶ is a hydrogen atom, X is a sulfur atom, and the combination of R¹, R², R³, R⁶, R⁷, and R⁸ is any one of the combinations shown in [Table L100] to [Table L180] (hereinafter, referred to as a compound group SX1550).

The compound (L-3), wherein R¹³ is a hydrogen atom, R¹⁴ is a chlorine atom, R¹⁵ is a hydrogen atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1551).

The compound (L-3), wherein R¹³ is a hydrogen atom, R¹⁴ is a hydrogen atom, R¹⁵ is a chlorine atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1552).

The compound (L-3), wherein R¹³ is a chlorine atom, R¹⁴ is a chlorine atom, R¹⁵ is a chlorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1553).

The compound (L-3), wherein R¹³ is a hydrogen atom, R¹⁴ is a chlorine atom, R¹⁵ is a chlorine atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1554).

The compound (L-3), wherein R¹³ is a bromine atom, R¹⁴ is a bromine atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1555).

The compound (L-3), wherein R¹³ is a bromine atom, R¹⁴ is a hydrogen atom, R¹⁵ is a bromine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1556).

The compound (L-3), wherein R¹³ is a bromine atom, R¹⁴ is a hydrogen atom, R¹⁵ is a hydrogen atom, R¹⁶ is a bromine atom, X is a sulfur atom, and the combination of R¹, R², R⁵, R⁶, R⁷, and R⁸ is any one of the combinations shown in [Table L100] to [Table L180] (hereinafter, referred to as a compound group SX1557).

The compound (L-3), wherein R¹³ is a hydrogen atom, R¹⁴ is a bromine atom, R¹⁵ is a bromine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1558).

The compound (L-3), wherein R¹³ is a hydrogen atom, R¹⁴ is a bromine atom, R¹⁵ is a hydrogen atom, R¹⁶ is a bromine atom, X is a sulfur atom, and the combination of R¹, R², R⁵, R⁶, R⁷, and R⁸ is any one of the combinations shown in [Table L100] to [Table L180] (hereinafter, referred to as a compound group SX1559).

The compound (L-3), wherein R¹³ is a hydrogen atom, R¹⁴ is a hydrogen atom, R¹⁵ is a bromine atom, R¹⁶ is a bromine atom, X is a sulfur atom, and the combination of R¹, R², R⁵, R⁶, R⁷, and R⁸ is any one of the combinations shown in [Table L100] to [Table L180] (hereinafter, referred to as a compound group SX1560).

The compound (L-3), wherein R¹³ is a bromine atom, R¹⁴ is a bromine atom, R¹⁵ is a bromine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1561).

The compound (L-3), wherein R¹³ is a hydrogen atom, R¹⁴ is a bromine atom, R¹⁵ is a bromine atom, R¹⁶ is a bromine atom, X is a sulfur atom, and the combination of R¹, R², R⁵, R⁶, R⁷, and R⁸ is any one of the combinations shown in [Table L100] to [Table L180] (hereinafter, referred to as a compound group SX1562).

The compound (L-3), wherein R¹³ is an iodine atom, R¹⁴ is an iodine atom, R¹⁵ is a hydrogen atom, Ru is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1563).

The compound (L-3), wherein R¹³ is an iodine atom, R¹⁴ is a hydrogen atom, R¹⁵ is an iodine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1564).

The compound (L-3), wherein R¹³ is an iodine atom, R¹⁴ is a hydrogen atom, R¹⁵ is a hydrogen atom, R¹⁶ is an iodine atom, X is a sulfur atom, and the combination of R¹, R², R⁵, R⁶, R⁷, and R⁸ is any one of the combinations shown in [Table L100] to [Table L180] (hereinafter, referred to as a compound group SX1565).

The compound (L-3), wherein R¹³ is a hydrogen atom, R¹⁴ is an iodine atom, R¹⁵ is an iodine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1566).

The compound (L-3), wherein R¹³ is a hydrogen atom, R¹⁴ is an iodine atom, R¹⁵ is a hydrogen atom, R¹⁶ is an iodine atom, X is a sulfur atom, and the combination of R¹, R², R⁵, R⁶, R⁷, and R⁸ is any one of the combinations shown in [Table L100] to [Table L180] (hereinafter, referred to as a compound group SX1567).

The compound (L-3), wherein R¹³ is a hydrogen atom, R¹⁴ is a hydrogen atom, R¹⁵ is an iodine atom, R¹⁶ is an iodine atom, X is a sulfur atom, and the combination of R¹, R², R⁵, R⁶, R⁷, and R⁸ is any one of the combinations shown in [Table L100] to [Table L180] (hereinafter, referred to as a compound group SX1568).

The compound (L-3), wherein R¹³ is an iodine atom, R¹⁴ is an iodine atom, R¹⁵ is an iodine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1569).

The compound (L-3), wherein R¹³ is a hydrogen atom, R¹⁴ is an iodine atom, R¹⁵ is an iodine atom, R¹⁶ is an iodine atom, X is a sulfur atom, and the combination of R¹, R², R⁵, R⁶, R⁷, and R⁸ is any one of the combinations shown in [Table L100] to [Table L180] (hereinafter, referred to as a compound group SX1570).

The compound (L-3), wherein R¹³ is a fluorine atom, R¹⁴ is a chlorine atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1571).

The compound (L-3), wherein R¹³ is a fluorine atom, R¹⁴ is a hydrogen atom, R¹⁵ is a chlorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1572).

The compound (L-3), wherein R¹³ is a fluorine atom, R¹⁴ is a hydrogen atom, R¹⁵ is a hydrogen atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1573).

The compound (L-3), wherein R¹³ is a chlorine atom, R¹⁴ is a fluorine atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1574).

The compound (L-3), wherein R¹³ is a chlorine atom, R¹⁴ is a hydrogen atom, R¹⁵ is a fluorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1575).

The compound (L-3), wherein R¹³ is a chlorine atom, R¹⁴ is a hydrogen atom, R¹⁵ is a hydrogen atom, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1576).

The compound (L-3), wherein R¹³ is a hydrogen atom, R¹⁴ is a fluorine atom, R¹⁵ is a chlorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1577).

The compound (L-3), wherein R¹³ is a hydrogen atom, R¹⁴ is a fluorine atom, R¹⁵ is a hydrogen atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1578).

The compound (L-3), wherein R¹³ is a hydrogen atom, R¹⁴ is a chlorine atom, R¹⁵ is a fluorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1579).

The compound (L-3), wherein R¹³ is a hydrogen atom, R¹⁴ is a chlorine atom, R¹⁵ is a hydrogen atom, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1580).

The compound (L-3), wherein R¹³ is a hydrogen atom, R¹⁴ is a hydrogen atom, R¹⁵ is a fluorine atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1581).

The compound (L-3), wherein R¹³ is a hydrogen atom, R¹⁴ is a hydrogen atom, R¹⁵ is a chlorine atom, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1582).

The compound (L-3), wherein R¹³ is a fluorine atom, R¹⁴ is a methyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1583).

The compound (L-3), wherein R¹³ is a fluorine atom, R¹⁴ is a hydrogen atom, R¹⁵ is a methyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1584).

The compound (L-3), wherein R¹³ is a fluorine atom, R¹⁴ is a hydrogen atom, R¹⁵ is a hydrogen atom, R¹⁶ is a methyl group, X is a sulfur atom, and the combination of R¹, R², R⁵, R⁶, R⁷, and R⁸ is any one of the combinations shown in [Table L100] to [Table L180] (hereinafter, referred to as a compound group SX1585).

The compound (L-3), wherein R¹³ is a methyl group, R¹⁴ is a fluorine atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1586).

The compound (L-3), wherein R¹³ is a methyl group, R¹⁴ is a hydrogen atom, R¹⁵ is a fluorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1587).

The compound (L-3), wherein R¹³ is a methyl group, R¹⁴ is a hydrogen atom, Ru5 is a hydrogen atom, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1588).

The compound (L-3), wherein R¹³ is a hydrogen atom, R¹⁴ is a fluorine atom, R¹⁵ is a methyl group, R¹⁶ is a hydrogen atom, X is a sulfur atom, and the combination of R¹, R², R³, R⁶, R⁷, and R⁸ is any one of the combinations shown in [Table L100] to [Table L180] (hereinafter, referred to as a compound group SX1589).

The compound (L-3), wherein R¹³ is a hydrogen atom, R¹⁴ is a fluorine atom, R¹⁵ is a hydrogen atom, R¹⁶ is a methyl group, X is a sulfur atom, and the combination of R¹, R², R⁵, R⁶, R⁷, and R⁸ is any one of the combinations shown in [Table L100] to [Table L180] (hereinafter, referred to as a compound group SX1590).

The compound (L-3), wherein R¹³ is a hydrogen atom, R¹⁴ is a methyl group, R¹⁵ is a fluorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1591).

The compound (L-3), wherein R¹³ is a hydrogen atom, R¹⁴ is a methyl group, R¹⁵ is a hydrogen atom, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1592).

The compound (L-3), wherein R¹³ is a hydrogen atom, R¹⁴ is a hydrogen atom, R¹⁵ is a fluorine atom, R¹⁶ is a methyl group, X is a sulfur atom, and the combination of R¹, R², R⁵, R⁶, R⁷, and R⁸ is any one of the combinations shown in [Table L100] to [Table L180] (hereinafter, referred to as a compound group SX1593).

The compound (L-3), wherein R¹³ is a hydrogen atom, R¹⁴ is a hydrogen atom, R¹⁵ is a methyl group, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1594).

The compound (L-3), wherein R¹³ is a fluorine atom, R¹⁴ is a methoxy group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1595).

The compound (L-3), wherein R¹³ is a fluorine atom, R¹⁴ is a hydrogen atom, R¹⁵ is a methoxy group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1596).

The compound (L-3), wherein R¹³ is a fluorine atom, R¹⁴ is a hydrogen atom, R¹⁵ is a hydrogen atom, R¹⁶ is a methoxy group, X is a sulfur atom, and the combination of R¹, R², R⁵, R⁶, R⁷, and R⁸ is any one of the combinations shown in [Table L100] to [Table L180] (hereinafter, referred to as a compound group SX1597).

The compound (L-3), wherein R¹³ is a methoxy group, R¹⁴ is a fluorine atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1598).

The compound (L-3), wherein R¹³ is a methoxy group, R¹⁴ is a hydrogen atom, R¹⁵ is a fluorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1599).

The compound (L-3), wherein R¹³ is a methoxy group, R¹⁴ is a hydrogen atom, R¹⁵ is a hydrogen atom, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1600).

The compound (L-3), wherein R¹³ is a hydrogen atom, R¹⁴ is a fluorine atom, R¹⁵ is a methoxy group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1601).

The compound (L-3), wherein R¹³ is a hydrogen atom, R¹⁴ is a fluorine atom, R¹⁵ is a hydrogen atom, R¹⁶ is a methoxy group, X is a sulfur atom, and the combination of R¹, R², R⁵, R⁶, R⁷, and R⁸ is any one of the combinations shown in [Table L100] to [Table L180] (hereinafter, referred to as a compound group SX1602).

The compound (L-3), wherein R¹³ is a hydrogen atom, R¹⁴ is a methoxy group, R¹⁵ is a fluorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1603).

The compound (L-3), wherein R¹³ is a hydrogen atom, R¹⁴ is a methoxy group, R¹⁵ is a hydrogen atom, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1604).

The compound (L-3), wherein R¹³ is a hydrogen atom, R¹⁴ is a hydrogen atom, R¹⁵ is a fluorine atom, R¹⁶ is a methoxy group, X is a sulfur atom, and the combination of R¹, R², R⁵, R⁶, R⁷, and R⁸ is any one of the combinations shown in [Table L100] to [Table L180] (hereinafter, referred to as a compound group SX1605).

The compound (L-3), wherein R¹³ is a hydrogen atom, R¹⁴ is a hydrogen atom, R¹⁵ is a methoxy group, R¹⁶ is a fluorine atom, X is a sulfur atom, and the combination of R¹, R², R⁵, R⁶, R⁷, and R⁸ is any one of the combinations shown in [Table L100] to [Table L180] (hereinafter, referred to as a compound group SX1606).

The compound (L-3), wherein R¹³ is a chlorine atom, R¹⁴ is a methyl group, R¹⁶ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1607).

The compound (L-3), wherein R¹³ is a chlorine atom, R¹⁴ is a hydrogen atom, R¹⁵ is a methyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1608).

The compound (L-3), wherein R¹³ is a chlorine atom, R¹⁴ is a hydrogen atom, R¹⁵ is a hydrogen atom, R¹⁶ is a methyl group, X is a sulfur atom, and the combination of R¹, R², R⁵, R⁶, R⁷, and R⁸ is any one of the combinations shown in [Table L100] to [Table L180] (hereinafter, referred to as a compound group SX1609).

The compound (L-3), wherein R¹³ is a methyl group, R¹⁴ is a chlorine atom, R¹⁵ is a hydrogen atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1610).

The compound (L-3), wherein R¹³ is a methyl group, R¹⁴ is a hydrogen atom, R¹⁵ is a chlorine atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1611).

The compound (L-3), wherein R¹³ is a methyl group, R¹⁴ is a hydrogen atom, R¹⁵ is a hydrogen atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1612).

The compound (L-3), wherein R¹³ is a hydrogen atom, R¹⁴ is a chlorine atom, R¹⁵ is a methyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1613).

The compound (L-3), wherein R¹³ is a hydrogen atom, R¹⁴ is a chlorine atom, R¹⁵ is a hydrogen atom, R¹⁶ is a methyl group, X is a sulfur atom, and the combination of R¹, R², R³, R⁶, R⁷, and R⁸ is any one of the combinations shown in [Table L100] to [Table L180] (hereinafter, referred to as a compound group SX1614).

The compound (L-3), wherein R¹³ is a hydrogen atom, R¹⁴ is a methyl group, R¹⁵ is a chlorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1615).

The compound (L-3), wherein R¹³ is a hydrogen atom, R¹⁴ is a methyl group, R¹⁵ is a hydrogen atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1616).

The compound (L-3), wherein R¹³ is a hydrogen atom, R¹⁴ is a hydrogen atom, R¹⁵ is a chlorine atom, R¹⁶ is a methyl group, X is a sulfur atom, and the combination of R¹, R², R⁵, R⁶, R⁷, and R⁸ is any one of the combinations shown in [Table L100] to [Table L180] (hereinafter, referred to as a compound group SX1617).

The compound (L-3), wherein R¹³ is a hydrogen atom, R¹⁴ is a hydrogen atom, R¹⁵ is a methyl group, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1618).

The compound (L-3), wherein R¹³ is a chlorine atom, R¹⁴ is a methoxy group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1619).

The compound (L-3), wherein R¹³ is a chlorine atom, R¹⁴ is a hydrogen atom, R¹⁵ is a methoxy group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1620).

The compound (L-3), wherein R¹³ is a chlorine atom, R¹⁴ is a hydrogen atom, R¹⁵ is a hydrogen atom, R¹⁶ is a methoxy group, X is a sulfur atom, and the combination of R¹, R², R⁵, R⁶, R⁷, and R⁸ is any one of the combinations shown in [Table L100] to [Table L180] (hereinafter, referred to as a compound group SX1621).

The compound (L-3), wherein R¹³ is a methoxy group, R¹⁴ is a chlorine atom, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1622).

The compound (L-3), wherein R¹³ is a methoxy group, R¹⁴ is a hydrogen atom, R¹⁵ is a chlorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1623).

The compound (L-3), wherein R¹³ is a methoxy group, R¹⁴ is a hydrogen atom, R¹⁵ is a hydrogen atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1624).

The compound (L-3), wherein R¹³ is a hydrogen atom, R¹⁴ is a chlorine atom, R¹⁵ is a methoxy group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1625).

The compound (L-3), wherein R¹³ is a hydrogen atom, R¹⁴ is a chlorine atom, R¹⁵ is a hydrogen atom, R¹⁶ is a methoxy group, X is a sulfur atom, and the combination of R¹, R², R⁵, R⁶, R⁷, and R⁸ is any one of the combinations shown in [Table L100] to [Table L180] (hereinafter, referred to as a compound group SX1626).

The compound (L-3), wherein R¹³ is a hydrogen atom, R¹⁴ is a methoxy group, R¹⁵ is a chlorine atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1627).

The compound (L-3), wherein R¹³ is a hydrogen atom, R¹⁴ is a methoxy group, R¹⁵ is a hydrogen atom, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1628).

The compound (L-3), wherein R¹³ is a hydrogen atom, R¹⁴ is a hydrogen atom, R¹⁵ is a chlorine atom, R¹⁶ is a methoxy group, X is a sulfur atom, and the combination of R¹, R², R⁵, R⁶, R⁷, and R⁸ is any one of the combinations shown in [Table L100] to [Table L180] (hereinafter, referred to as a compound group SX1629).

The compound (L-3), wherein R¹³ is a hydrogen atom, R¹⁴ is a hydrogen atom, R¹⁵ is a methoxy group, R¹⁶ is a chlorine atom, X is a sulfur atom, and the combination of R¹, R², R⁵, R⁶, R⁷, and R⁸ is any one of the combinations shown in [Table L100] to [Table L180] (hereinafter, referred to as a compound group SX1630).

The compound (L-3), wherein R¹³ is a methyl group, R¹⁴ is a methoxy group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1631).

The compound (L-3), wherein R¹³ is a methyl group, R¹⁴ is a hydrogen atom, R¹⁵ is a methoxy group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1632).

The compound (L-3), wherein R¹³ is a methyl group, R¹⁴ is a hydrogen atom, R¹⁵ is a hydrogen atom, R¹⁶ is a methoxy group, X is a sulfur atom, and the combination of R¹, R², R⁵, R⁶, R⁷, and R⁸ is any one of the combinations shown in [Table L100] to [Table L180] (hereinafter, referred to as a compound group SX1633).

The compound (L-3), wherein R¹³ is a methoxy group, R¹⁴ is a methyl group, R¹⁵ is a hydrogen atom, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1634).

The compound (L-3), wherein R¹³ is a methoxy group, R¹⁴ is a hydrogen atom, R¹⁵ is a methyl group, R¹⁶ is a hydrogen atom, X is a sulfur atom, and the combination of R¹, R², R⁵, R⁶, R⁷, and R⁸ is any one of the combinations Shown in [Table L100] to [Table L180] (hereinafter, referred to as a compound group SX1635).

The compound (L-3), wherein R¹³ is a methoxy group, R¹⁴ is a hydrogen atom, R¹⁵ is a hydrogen atom, R¹⁶ is a methyl group, X is a sulfur atom, and the combination of R¹, R², R⁵, R⁶, R⁷, and R⁸ is any one of the combinations shown in [Table L100] to [Table L180] (hereinafter, referred to as a compound group SX1636).

The compound (L-3), wherein R¹³ is a hydrogen atom, R¹⁴ is a methyl group, R¹⁵ is a methoxy group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1637).

The compound (L-3), wherein R¹³ is a hydrogen atom, R¹⁴ is a methyl group, R¹⁵ is a hydrogen atom, R¹⁶ is a methoxy group, X is a sulfur atom, and the combination of R¹, R², R⁵, R⁶, R⁷, and R⁸ is any one of the combinations shown in [Table L100] to [Table L180] (hereinafter, referred to as a compound group SX1638).

The compound (L-3), wherein R¹³ is a hydrogen atom, R¹⁴ is a methoxy group, R¹⁵ is a methyl group, R¹⁶ is a hydrogen atom, X is a sulfur atom, and the combination of R¹, R², R⁵, R⁶, R⁷, and R⁸ is any one of the combinations shown in [Table L100] to [Table L180] (hereinafter, referred to as a compound group SX1639).

The compound (L-3), wherein R¹³ is a hydrogen atom, R¹⁴ is a methoxy group, R¹⁵ is a hydrogen atom, R¹⁶ is a methyl group, X is a sulfur atom, and the combination of R¹, R², R⁵, R⁶, R⁷, and R⁸ is any one of the combinations shown in [Table L100] to [Table L180] (hereinafter, referred to as a compound group SX1640).

The compound (L-3), wherein R¹³ is a hydrogen atom, R¹⁴ is a hydrogen atom, R¹⁵ is a methyl group, R¹⁶ is a methoxy group, X is a sulfur atom, and the combination of R¹, R², R⁵, R⁶, R⁷, and R⁸ is any one of the combinations shown in [Table L100] to [Table L180] (hereinafter, referred to as a compound group SX1641).

The compound (L-3), wherein R¹³ is a hydrogen atom, R¹⁴ is a hydrogen atom, R¹⁵ is a methoxy group, R¹⁶ is a methyl group, X is a sulfur atom, and the combination of R¹, R², R⁵, R⁶, R⁷, and R⁸ is any one of the combinations shown in [Table L100] to [Table L180] (hereinafter, referred to as a compound group SX1642).

The compound (L-3), wherein R¹³ is a hydrogen atom, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1643).

The compound (L-3), wherein R¹³ is a methyl group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1644).

The compound (L-3), wherein R¹³ is a hydrogen atom, R¹⁴ is a methyl group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1645).

The compound (L-3), wherein R¹³ is a hydrogen atom, R¹⁴ is a hydrogen atom, R¹⁵ is a methyl group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1646).

The compound (L-3), wherein R¹³ is a hydrogen atom, R¹⁴ is a hydrogen atom, R¹⁵ is a hydrogen atom, R¹⁶ is a methyl group, X is NH, and the combination of R¹, R², R⁵, R⁶, R⁷, and R⁸ is any one of the combinations shown in [Table L100] to [Table L180] (hereinafter, referred to as a compound group SX1647).

The compound (L-3), wherein R¹³ is a methyl group, R¹⁴ is a methyl group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1648).

The compound (L-3), wherein R¹³ is a methyl group, R¹⁴ is a hydrogen atom, Ru is a methyl group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1649).

The compound (L-3), wherein R¹³ is a methyl group, R¹⁴ is a hydrogen atom, R¹⁵ is a hydrogen atom, R¹⁶ is a methyl group, X is NH, and the combination of R¹, R², R⁵, R⁶, R⁷, and R⁸ is any one of the combinations shown in [Table L100] to [Table L180] (hereinafter, referred to as a compound group SX1650).

The compound (L-3), wherein R¹³ is a hydrogen atom, R¹⁴ is a methyl group, R¹⁵ is a methyl group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1651).

The compound (L-3), wherein R¹³ is a hydrogen atom, R¹⁴ is a methyl group, R¹⁵ is a hydrogen atom, R¹⁶ is a methyl group, X is NH, and the combination of R¹, R², R⁵, R⁶, R⁷, and R⁸ is any one of the combinations shown in [Table L100] to [Table L180] (hereinafter, referred to as a compound group SX1652).

The compound (L-3), wherein R¹³ is a hydrogen atom, R¹⁴ is a hydrogen atom, R¹⁵ is a methyl group, R¹⁶ is a methyl group, X is NH, and the combination of R¹, R², R⁵, R⁶, R⁷, and R⁸ is any one of the combinations shown in [Table L100] to [Table L180] (hereinafter, referred to as a compound group SX1653).

The compound (L-3), wherein R¹³ is a methyl group, R¹⁴ is a methyl group, R¹⁵ is a methyl group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1654).

The compound (L-3), wherein R¹³ is a hydrogen atom, R¹⁴ is a methyl group, R¹⁵ is a methyl group, R¹⁶ is a methyl group, X is NH, and the combination of R¹, R², R⁵, R⁶, R⁷, and R⁸ is any one of the combinations shown in [Table L100] to [Table L180] (hereinafter, referred to as a compound group SX1655).

The compound (L-3), wherein R¹³ is a fluorine atom, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1656).

The compound (L-3), wherein R¹³ is a hydrogen atom, R¹⁴ is a fluorine atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1657).

The compound (L-3), wherein R¹³ is a hydrogen atom, R¹⁴ is a hydrogen atom, R¹⁵ is a fluorine atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1658).

The compound (L-3), wherein R¹³ is a hydrogen atom, R¹⁴ is a hydrogen atom, R¹⁵ is a hydrogen atom, R¹⁶ is a fluorine atom, X is NH, and the combination of R¹, R², R⁵, R⁶, R⁷, and R⁸ is any one of the combinations shown in [Table L100] to [Table L180] (hereinafter, referred to as a compound group SX1659).

The compound (L-3), wherein R¹³ is a chlorine atom, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1660).

The compound (L-3), wherein R¹³ is a hydrogen atom, R¹⁴ is a chlorine atom, R¹⁵ is a hydrogen atom, R¹⁶ is a hydrogen atom, X is NH, and the combination of R¹, R², R⁵, R⁶, R⁷, and R^{a} is any one of the combinations shown in [Table L100] to [Table L180] (hereinafter, referred to as a compound group SX1661).

The compound (L-3), wherein R¹³ is a hydrogen atom, R¹⁴ is a hydrogen atom, R¹⁵ is a chlorine atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1662).

The compound (L-3), wherein R¹³ is a hydrogen atom, R¹⁴ is a hydrogen atom, R¹⁵ is a hydrogen atom, R¹⁶ is a chlorine atom, X is NH, and the combination of R¹, R², R⁵, R⁶, R⁷, and R⁸ is any one of the combinations shown in [Table L100] to [Table L180] (hereinafter, referred to as a compound group SX1663).

The compound (L-3), wherein R¹³ is a bromine atom, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1664).

The compound (L-3), wherein R¹³ is a bromine atom, R¹⁴ R¹⁴ is a bromine atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1665).

The compound (L-3), wherein R¹³ is a hydrogen atom, R¹⁴ is a hydrogen atom, R¹⁵ is a bromine atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1666).

The compound (L-3), wherein R¹³ is a hydrogen atom, R¹⁴ is a hydrogen atom, R¹⁵ is a hydrogen atom, R¹⁶ is a bromine atom, X is NH, and the combination of R¹, R², R⁵, R⁶, R⁷, and R⁸ is any one of the combinations shown in [Table L100] to [Table L180] (hereinafter, referred to as a compound group SX1667).

The compound (L-3), wherein R¹³ is an iodine atom, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1668).

The compound (L-3), wherein R¹³ is a hydrogen atom, R¹⁴ is an iodine atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1669).

The compound (L-3), wherein R¹³ is a hydrogen atom, R¹⁴ is a hydrogen atom, R¹⁵ is an iodine atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1670).

The compound (L-3), wherein R¹³ is a hydrogen atom, R¹⁴ is a hydrogen atom, R¹⁵ is a hydrogen atom, R¹⁶ is an iodine atom, X is NH, and the combination of R¹, R², R⁵, R⁶, R⁷, and R⁸ is any one of the combinations shown in [Table L13] to [Table L180] (hereinafter, referred to as a compound group SX1671).

The compound (L-3), wherein R¹³ is a methoxy group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1672).

The compound (L-3), wherein R¹³ is a hydrogen atom, R¹⁴ is a methoxy group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1673).

The compound (L-3), wherein R¹³ is a hydrogen atom, R¹⁴ is a hydrogen atom, R¹⁵ is a methoxy group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1674).

The compound (L-3), wherein R¹³ is a hydrogen atom, R¹⁴ is a hydrogen atom, R¹⁵ is a hydrogen atom, R¹⁶ is a methoxy group, X is NH, and the combination of R¹, R², R⁵, R⁶, R⁷, and R⁸ is any one of the combinations shown in [Table L100] to [Table L180] (hereinafter, referred to as a compound group SX1675).

The compound (L-3), wherein R¹³ is a trifluoromethyl group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1676).

The compound (L-3), wherein R¹³ is a hydrogen atom, R¹⁴ is a trifluoromethyl group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1677).

The compound (L-3), wherein R¹³ is a hydrogen atom, R¹⁴ is a hydrogen atom, R¹⁵ is a trifluoromethyl group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1678).

The compound (L-3), wherein R¹³ is a hydrogen atom, R¹⁴ is a hydrogen atom, R¹⁵ is a hydrogen atom, R¹⁶ is a trifluoromethyl group, X is NH, and the combination of R¹, R², R⁵, R⁶, R⁷, and R⁸ is any one of the combinations shown in [Table L100] to [Table L180] (hereinafter, referred to as a compound group SX1679).

The compound (L-3), wherein R¹³ is a cyclopropyl. group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1680).

The compound (L-3), wherein R¹³ is a hydrogen atom, R¹⁴ is a cyclopropyl group, Rib is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1681).

The compound (L-3), wherein R¹³ is a hydrogen atom, R¹⁴ is a hydrogen atom, R¹⁵ is a cyclopropyl group, R¹⁶ is a hydrogen atom, X is NH, and the combination of R¹, R², R⁵, R⁶, R⁷, and R⁸ is any one of the combinations shown in [Table L100] to [Table 1L180] (hereinafter, referred to as a compound group SX1682).

The compound (L-3), wherein R¹³ is a hydrogen atom, R¹⁴ is a hydrogen atom, R¹⁵ is a hydrogen atom, R¹⁶ is a cyclopropyl group, X is NH, and the combination of R¹, R², R⁵, R⁶, R⁷, and R⁸ is any one of the combinations shown in [Table L100] to [Table L180] (hereinafter, referred to as a compound group SX1683).

The compound (L-3), wherein R¹³ is a cyclobutyl group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1684).

The compound (L-3), wherein R¹³ is a hydrogen atom, R¹⁴ is a cyclobutyl group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1685).

The compound (L-3), wherein R¹³ is a hydrogen atom, R¹⁴ is a hydrogen atom, R¹⁵ is a cyclobutyl group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1686).

The compound (L-3), wherein R¹³ is a hydrogen atom, R¹⁴ is a hydrogen atom, R¹⁵ is a hydrogen atom, R¹⁶ is a cyclobutyl group, X is NH, and the combination of R¹, R², R⁵, R⁶, R⁷, and R⁸ is any one of the combinations shown in [Table L100] to [Table L180] (hereinafter, referred to as a compound group SX1687).

The compound (L-3), wherein R¹³ is a cyclopentyl group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1688).

The compound (L-3), wherein R¹³ is a hydrogen atom, R¹⁴ is a cyclopentyl group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1689).

The compound (L-3), wherein R¹³ is a hydrogen atom, R¹⁴ is a hydrogen atom, R¹⁵ is a cyclopentyl group, R¹⁶ is a hydrogen atom, X is NH, and the combination of R¹, R², R⁵, R⁶, R⁷, and R⁹ is any one of the combinations shown in [Table L100] to [Table L180] (hereinafter, referred to as a compound group SX1690).

The compound (L-3), wherein R¹³ is a hydrogen atom, R¹⁴ is a hydrogen atom, R¹⁵ is a hydrogen atom, R¹⁶ is a cyclopentyl group, X is NH, and the combination of R¹, R², R⁵, R⁶, R⁷, and R⁸ is any one of the combinations shown in [Table L100] to [Table L180] (hereinafter, referred to as a compound group SX1691).

The compound (L-3), wherein R¹³ is a cyclohexyl group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1692).

The compound (L-3), wherein R¹³ is a hydrogen atom, R¹⁴ is a cyclohexyl group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1693).

The compound (L-3), wherein R¹³ is a hydrogen atom, R¹⁴ is a hydrogen atom, R¹⁵ is a cyclohexyl group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1694).

The compound (L-3), wherein R¹³ is a hydrogen atom, R¹⁴ is a hydrogen atom, R¹⁵ is a hydrogen atom, R¹⁶ is a cyclohexyl group, X is NH, and the combination of R¹, R², R⁵, R⁶, R⁷, and R⁸ is any one of the combinations shown in [Table L100] to [Table L180] (hereinafter, referred to as a compound group SX1695).

The compound (L-3), wherein R¹³ is a phenyl group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1696).

The compound (L-3), wherein R¹³ is a hydrogen atom, R¹⁴ is a phenyl group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1697).

The compound (L-3), wherein R¹³ is a hydrogen atom, R¹⁴ is a hydrogen atom, R¹⁵ is a phenyl group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1698).

The compound (L-3), wherein R¹³ is a hydrogen atom, R¹⁴ is a hydrogen atom, R¹⁵ is a hydrogen atom, R¹⁶ is a phenyl group, X is NH, and the combination of R¹, R², R⁵, R⁶, R⁷, and R⁸ is any one of the combinations shown in [Table L100] to [Table L180] (hereinafter, referred to as a compound group SX1699).

The compound (L-3), wherein R¹³ is an acetyl group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1700).

The compound (L-3), wherein R¹³ is a hydrogen atom, R¹⁴ is an acetyl group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1701).

The compound (L-3), wherein R¹³ is a hydrogen atom, R¹⁴ is a hydrogen atom, R¹⁵ is an acetyl group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1702).

The compound (L-3), wherein R¹³ is a hydrogen atom, R¹⁴ is a hydrogen atom, R¹⁵ is a hydrogen atom, R¹⁶ is an acetyl group, X is NH, and the combination of R¹, R², R⁵, R⁶, R⁷, and R⁸ is any one of the combinations shown in [Table L100] to [Table L180] (hereinafter, referred to as a compound group SX1703).

The compound (L-3), wherein R¹³ is a trifluoroacetyl group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1704).

The compound (L-3), wherein R¹³ is a hydrogen atom, R¹⁴ is a trifluoroacetyl group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1705).

The compound (L-3), wherein R¹³ is a hydrogen atom, R¹⁴ is a hydrogen atom, R¹⁵ is a trifluoroacetyl group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1706).

The compound (L-3), wherein R¹³ is a hydrogen atom, R¹⁴ is a hydrogen atom, R¹⁵ is a hydrogen atom, R¹⁶ is a trifluoroacetyl group, X is NH, and the combination of R¹, R², R⁵, R⁶, R⁷, and R⁸ is any one of the combinations shown in [Table L100] to [Table L180] (hereinafter, referred to as a compound group SX1707).

The compound (L-3), wherein R¹³ is a methoxycarbonyl group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1708).

The compound (L-3), wherein R¹³ is a hydrogen atom, R¹⁴ is a methoxycarbonyl group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1709).

The compound (L-3), wherein R¹³ is a hydrogen atom, R¹⁴ is a hydrogen atom, R¹⁵ is a methoxycarbonyl group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1710).

The compound (L-3), wherein R¹³ is a hydrogen atom, R¹⁴ is a hydrogen atom, R¹⁵ is a hydrogen atom, R¹⁶ is a methoxycarbonyl group, X is NH, and the combination of R¹, R², R⁵, R⁶, R⁷, and R⁸ is any one of the combinations shown in [Table L100] to [Table L180] (hereinafter, referred to as a compound group SX1711).

The compound (L-3), wherein R¹³ is a trifluoromethoxycarbonyl group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1712).

The compound (L-3), wherein R¹³ is a hydrogen atom, R¹⁴ is a trifluoromethoxycarbonyl group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1713).

The compound (L-3), wherein R¹³ is a hydrogen atom, R¹⁴ is a hydrogen atom, R¹⁵ is a trifluoromethoxycarbonyl group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1714).

The compound (L-3), wherein R¹³ is a hydrogen atom, R¹⁴ is a hydrogen atom, R¹⁵ is a hydrogen atom, R¹⁶ is a trifluoromethoxycarbonyl group, X is NH, and the combination of R¹, R², R⁵, R⁶, R⁷, and R⁸ is any one of the combinations shown in [Table L100] to [Table L180] (hereinafter, referred to as a compound group SX1715).

The compound (L-3), wherein R¹³ is an acetoxy group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1716).

The compound (L-3), wherein R¹³ is a hydrogen atom, R¹⁴ is an acetoxy group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1717).

The compound (L-3), wherein R¹³ is a hydrogen atom, R¹⁴ is a hydrogen atom, R¹⁵ is an acetoxy group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1718).

The compound (L-3), wherein R¹³ is a hydrogen atom, R¹⁴ is a hydrogen atom, R¹⁵ is a hydrogen atom, R¹⁶ is an acetoxy group, X is NH, and the combination of R¹, R², R⁵, R⁶, R⁷, and R⁸ is any one of the combinations shown in [Table L100] to [Table L180] (hereinafter, referred to as a compound group SX1719).

The compound (L-3), wherein R¹³ is a trifluoroacetoxy group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1720).

The compound (L-3), wherein R¹³ is a hydrogen atom, R¹⁴ is a trifluoroacetoxy group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1721).

The compound (L-3), wherein R¹³ is a hydrogen atom, R¹⁴ is a hydrogen atom, R¹⁵ is a trifluoroacetoxy group, Rie is a hydrogen atom, X is NH, and the combination of R¹, R², R⁵, R⁶, R⁷, and R⁸ is any one of the combinations shown in [Table L100] to [Table L180] (hereinafter, referred to as a compound group SX1722).

The compound (L-3), wherein R¹³ is a hydrogen atom, R¹⁴ is a hydrogen atom, R¹⁵ is a hydrogen atom, R¹⁶ is a trifluoroacetoxy group, X is NH, and the combination of R¹, R², R⁵, R⁶, R⁷, and R⁸ is any one of the combinations shown in [Table L100] to [Table L180] (hereinafter, referred to as a compound group SX1723).

The compound (L-3), wherein R¹³ is a cyano group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1724).

The compound (L-3), wherein R¹³ is a hydrogen atom, R¹⁴ is a cyano group, R¹⁵ is a hydrogen atom, R¹⁶ is a hydrogen atom, X is NH, and the combination of R¹, R², R⁵, R⁶, R⁷, and R^{a} is any one of the combinations shown in [Table L100] to [Table L180] (hereinafter, referred to as a compound group SX1725).

The compound (L-3), wherein R¹³ is a hydrogen atom, R¹⁴ is a hydrogen atom, R¹⁵ is a cyano group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1726).

The compound (L-3), wherein R¹³ is a hydrogen atom, R¹⁴ is a hydrogen atom, R¹⁵ is a hydrogen atom, R¹⁶ is a cyano group, X is NH, and the combination of R¹, R², R⁵, R⁶, R⁷, and R⁸ is any one of the combinations shown in [Table L100] to [Table L180] (hereinafter, referred to as a compound group SX1727).

The compound (L-3), wherein R¹³ is a formyl group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1728).

The compound (L-3), wherein R¹³ is a hydrogen atom, R¹⁴ is a formyl group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1729).

The compound (L-3), wherein R¹³ is a hydrogen atom, R¹⁴ is a hydrogen atom, R¹⁵ is a formyl group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1730).

The compound (L-3), wherein R¹³ is a hydrogen atom, R¹⁴ is a hydrogen atom, R¹⁵ is a hydrogen atom, R¹⁶ is a formyl group, X is NH, and the combination of R¹, R², R⁵, R⁶, R⁷, and R⁸ is any one of the combinations shown in [Table L100] to [Table L180] (hereinafter, referred to as a compound group SX1731).

The compound (L-3), wherein R¹³ is a carboxy group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1732).

The compound (L-3), wherein R¹³ is a hydrogen atom, R¹⁴ is a carboxy group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1733).

The compound (L-3), wherein R¹³ is a hydrogen atom, R¹⁴ is a hydrogen atom, R¹⁵ is a carboxy group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1734).

The compound (L-3), wherein R¹³ is a hydrogen atom, R¹⁴ is a hydrogen atom, R¹⁵ is a hydrogen atom, R¹⁶ is a carboxy group, X is NH, and the combination of R¹, R², R⁵, R⁶, R⁷, and R⁸ is any one of the combinations shown in [Table L100] to [Table L180] (hereinafter, referred to as a compound group SX1735).

The compound (L-3), wherein R¹³ is a nitro group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1736).

The compound (L-3), wherein R¹³ is a hydrogen atom, R¹⁴ is a nitro group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1737).

The compound (L-3), wherein R¹³ is a hydrogen atom, R¹⁴ is a hydrogen atom, R¹⁵ is a nitro group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1738).

The compound (L-3), wherein R¹³ is a hydrogen atom, R¹⁴ is a hydrogen atom, R¹⁵ is a hydrogen atom, R¹⁶ is a nitro group, X is NH, and the combination of R¹, R², R⁵, R⁶, R⁷, and R⁸ is any one of the combinations shown in [Table L100] to [Table L180] (hereinafter, referred to as a compound group SX1739).

The compound (L-3), wherein R¹³ is a hydroxy group, R¹⁴ is a hydrogen atom, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1740).

The compound (L-3), wherein R¹³ is a hydrogen atom, R¹⁴ is a hydroxy group, R¹⁵ is a hydrogen atom, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1741).

The compound (L-3), wherein R¹³ is a hydrogen atom, R¹⁴ is a hydrogen atom, R¹⁵ is a hydroxy group, R¹⁶ is a hydrogen atom, X is NH, and the combination of R¹, R², R⁵, R⁶, R⁷, and R⁸ is any one of the combinations shown in [Table L100] to [Table L180] (hereinafter, referred to as a compound group SX1742).

The compound (L-3), wherein R¹³ is a hydrogen atom, R¹⁴ is a hydrogen atom, R¹⁵ is a hydrogen atom, R¹⁶ is a hydroxy group, X is NH, and the combination of R¹, R², R⁵, R⁶, R⁷, and R⁸ is any one of the combinations shown in [Table L100] to [Table L180] (hereinafter, referred to as a compound group SX1743).

The compound (L-3), wherein R¹³ is a hydrogen atom, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1744).

The compound (L-3), wherein R¹³ is a methyl group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1745).

The compound (L-3), wherein R¹³ is a hydrogen atom, R¹⁴ is a methyl group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1746).

The compound (L-3), wherein R¹³ is a hydrogen atom, R¹⁴ is a hydrogen atom, R¹⁵ is a methyl group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1747).

The compound (L-3), wherein R¹³ is a hydrogen atom, R¹⁴ is a hydrogen atom, R¹⁵ is a hydrogen atom, R¹⁶ is a methyl group, X is NCH₃, and the combination of R¹, R², R⁵, R⁶, R⁷, and R⁸ is any one of the combinations shown in [Table L100] to [Table L180] (hereinafter, referred to as a compound group SX1748).

The compound (L-3), wherein R¹³ is a methyl group, R¹⁴ is a methyl group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1749).

The compound (L-3), wherein R¹³ is a methyl group, R¹⁴ is a hydrogen atom, R¹⁵ is a methyl group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1750).

The compound (L-3), wherein R¹³ is a methyl group, R¹⁴ is a hydrogen atom, R¹⁵ is a hydrogen atom, R¹⁶ is a methyl group, X is NCH₃, and the combination of R¹, R², R³, R⁶, R⁷, and R⁸ is any one of the combinations shown in [Table L100] to [Table L180] (hereinafter, referred to as a compound group SX1751).

The compound (L-3), wherein R¹³ is a hydrogen atom, R¹⁴ is a methyl group, R¹⁵ is a methyl group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1752).

The compound (L-3), wherein R¹³ is a hydrogen atom, R¹⁴ is a methyl group, R¹⁵ is a hydrogen atom, R¹⁶ is a methyl group, X is NCH₃, and the combination of R¹, R², R⁵, R⁶, R⁷, and R⁸ is any one of the combinations shown in [Table L100] to [Table L180] (hereinafter, referred to as a compound group SX1753).

The compound (L-3), wherein R¹³ is a hydrogen atom, R¹⁴ is a hydrogen atom, R¹⁵ is a methyl group, R¹⁶ is a methyl group, X is NCH₃, and the combination of R¹, R², R⁵, R⁶, R⁷, and R⁸ is any one of the combinations shown in [Table L100] to [Table L180] (hereinafter, referred to as a compound group SX1754).

The compound (L-3), wherein R¹³ is a methyl group, R¹⁴ is a methyl group, R¹⁵ is a methyl group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1755).

The compound (L-3), wherein R¹³ is a hydrogen atom, R¹⁴ is a methyl group, R¹⁵ is a methyl group, R¹⁶ is a methyl group, X is NCH₃, and the combination of R¹, R², R⁵, R⁶, R⁷, and R⁸ is any one of the combinations shown in [Table L100] to [Table L180] (hereinafter, referred to as a compound group SX1756).

The compound (L-3), wherein R¹³ is a fluorine atom, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1757).

The compound (L-3), wherein R¹³ is a hydrogen atom, R¹⁴ is a fluorine atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1758).

The compound (L-3), wherein R¹³ is a hydrogen atom, R¹⁴ is a hydrogen atom, R¹⁵ is a fluorine atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1759).

The compound (L-3), wherein R¹³ is a hydrogen atom, R¹⁴ is a hydrogen atom, R¹⁵ is a hydrogen atom, R¹⁶ is a fluorine atom, X is NCH₃, and the combination of R¹, R², R⁵, R⁶, R⁷, and R⁸ is any one of the combinations shown in [Table L100] to [Table L180] (hereinafter, referred to as a compound group SX1760).

The compound (L-3), wherein R¹³ is a chlorine atom, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1761).

The compound (L-3), wherein R¹³ is a hydrogen atom, R¹⁴ is a chlorine atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1762).

The compound (L-3), wherein R¹³ is a hydrogen atom, R¹⁴ is a hydrogen atom, R¹⁵ is a chlorine atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1763).

The compound (L-3), wherein R¹³ is a hydrogen atom, R¹⁴ is a hydrogen atom, R¹⁵ is a hydrogen atom, R¹⁶ is a chlorine atom, X is NCH₃, and the combination of R¹, R², R⁵, R⁶, R⁷, and R⁸ is any one of the combinations shown in [Table L100] to [Table L180] (hereinafter, referred to as a compound group SX1764).

The compound (L-3), wherein R¹³ is a bromine atom, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1765).

The compound (L-3), wherein R¹³ is a hydrogen atom, R¹⁴ is a bromine atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1766).

The compound (L-3), wherein R¹³ is a hydrogen atom, R¹⁴ is a hydrogen atom, R¹⁵ is a bromine atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1767).

The compound (L-3), wherein R¹³ is a hydrogen atom, R¹⁴ is a hydrogen atom, R¹⁵ is a hydrogen atom, R¹⁶ is a bromine atom, X is NCH₃, and the combination of R¹, R², R⁵, R⁶, R⁷, and R⁸ is any one of the combinations shown in [Table L100] to [Table L180] (hereinafter, referred to as a compound group SX1768).

The compound (L-3), wherein R¹³ is an iodine atom, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1769).

The compound (L-3), wherein R¹³ is a hydrogen atom, R¹⁴ is an iodine atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1770).

The compound (L-3), wherein R¹³ is a hydrogen atom, R¹⁴ is a hydrogen atom, R¹⁵ is an iodine atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1771).

The compound (L-3), wherein R¹³ is a hydrogen atom, R¹⁴ is a hydrogen atom, R¹⁵ is a hydrogen atom, R¹⁶ is an iodine atom, X is NCH₃, and the combination of R¹, R², R⁵, R⁶, R⁷, and R⁸ is any one of the combinations shown in [Table L100] to [Table L180] (hereinafter, referred to as a compound group SX1772).

The compound (L-3), wherein R¹³ is a methoxy group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1773).

The compound (L-3), wherein R¹³ is a hydrogen atom, R¹⁴ is a methoxy group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1774).

The compound (L-3), wherein R¹³ is a hydrogen atom, R¹⁴ is a hydrogen atom, R¹⁵ is a methoxy group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1775).

The compound (L-3), wherein R¹³ is a hydrogen atom, R¹⁴ is a hydrogen atom, R¹⁵ is a hydrogen atom, R¹⁶ is a methoxy group, X is NCH₃, and the combination of R¹, R², R⁵, R⁶, R⁷, and R⁸ is any one of the combinations shown in [Table L100] to [Table L180] (hereinafter, referred to as a compound group SX1776).

The compound (L-3), wherein R¹³ is a trifluoromethyl group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1777).

The compound (L-3), wherein R¹³ is a hydrogen atom, R¹⁴ is a trifluoromethyl group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1778).

The compound (L-3), wherein R¹³ is a hydrogen atom, R¹⁴ is a hydrogen atom, R¹⁵ is a trifluoromethyl group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1779).

The compound (L-3), wherein R¹³ is a hydrogen atom, R¹⁴ is a hydrogen atom, R¹⁵ is a hydrogen atom, R¹⁶ is a trifluoromethyl group, X is NCH₃, and the combination of R¹, R², R⁵, R⁶, R⁷, and R⁸ is any one of the combinations shown in [Table L100] to [Table L180] (hereinafter, referred to as a compound group SX1780).

The compound (L-3), wherein R¹³ is a cyclopropyl group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1781).

The compound (L-3), wherein R¹³ is a hydrogen atom, R¹⁴ is a cyclopropyl group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1782).

The compound (L-3), wherein R¹³ is a hydrogen atom, R¹⁴ is a hydrogen atom, R¹⁵ is a cyclopropyl group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1783).

The compound (L-3), wherein R¹³ is a hydrogen atom, R¹⁴ is a hydrogen atom, R¹⁵ is a hydrogen atom, R¹⁶ is a cyclopropyl group, X is NCH₃, and the combination of R¹, R², R⁵, R⁶, R⁷, and R⁸ is any one of the combinations shown in [Table L100] to [Table L180] (hereinafter, referred to as a compound group SX1784).

The compound (L-3), wherein R¹³ is a cyclobutyl group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1785).

The compound (L-3), wherein R¹³ is a hydrogen atom, R¹⁴ is a cyclobutyl group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1786).

The compound (L-3), wherein R¹³ is a hydrogen atom, R¹⁴ is a hydrogen atom, R¹⁵ is a cyclobutyl group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1787).

The compound (L-3), wherein R¹³ is a hydrogen atom, R¹⁴ is a hydrogen atom, R¹⁵ is a hydrogen atom, R¹⁶ is a cyclobutyl group, X is NCH₃, and the combination of R¹, R², R⁵, R⁶, R⁷, and R⁸ is any one of the combinations shown in [Table L100] to [Table L180] (hereinafter, referred to as a compound group SX1788).

The compound (L-3), wherein R¹³ is a cyclopentyl group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1789).

The compound (L-3), wherein R¹³ is a hydrogen atom, R¹⁴ is a cyclopentyl group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1790).

The compound (L-3), wherein R¹³ is a hydrogen atom, R¹⁴ is a hydrogen atom, R¹⁵ is a cyclopentyl group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1791).

The compound (L-3), wherein R¹³ is a hydrogen atom, R¹⁴ is a hydrogen atom, R¹⁵ is a hydrogen atom, R¹⁶ is a cyclopentyl group, X is NCH₃, and the combination of R¹, R², R⁵, R⁶, R⁷, and R⁸ is any one of the combinations shown in [Table L100] to [Table L180] (hereinafter, referred to as a compound group SX1792).

The compound (L-3), wherein R¹³ is a cyclohexyl group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1793).

The compound (L-3), wherein R¹³ is a hydrogen atom, R¹⁴ is a cyclohexyl group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1794).

The compound (L-3), wherein R¹³ is a hydrogen atom, R¹⁴ is a hydrogen atom, R¹⁵ is a cyclohexyl group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1795).

The compound (L-3), wherein R¹³ is a hydrogen atom, R¹⁴ is a hydrogen atom, R¹⁵ is a hydrogen atom, R¹⁶ is a cyclohexyl group, X is NCH₃, and the combination of R¹, R², R⁵, R⁶, R⁷, and R⁸ is any one of the combinations shown in [Table L100] to [Table L180] (hereinafter, referred to as a compound group SX1796).

The compound (L-3), wherein R¹³ is a phenyl group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1797).

The compound (L-3), wherein R¹³ is a hydrogen atom, R¹⁴ is a phenyl group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1798).

The compound (L-3), wherein R¹³ is a hydrogen atom, R¹⁴ is a hydrogen atom, R¹⁵ is a phenyl group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1799).

The compound (L-3), wherein R¹³ is a hydrogen atom, R¹⁴ is a hydrogen atom, R¹⁵ is a hydrogen atom, R¹⁶ is a phenyl group, X is NCH₃, and the combination of R¹, R², R⁵, R⁶, R⁷, and R⁸ is any one of the combinations shown in [Table L100] to [Table L180] (hereinafter, referred to as a compound group SX1800).

The compound (L-3), wherein R¹³ is an acetyl group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1801).

The compound (L-3), wherein R¹³ is a hydrogen atom, R¹⁴ is an acetyl group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1802).

The compound (L-3), wherein R¹³ is a hydrogen atom, R¹⁴ is a hydrogen atom, R¹⁵ is an acetyl group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1803).

The compound (L-3), wherein R¹³ is a hydrogen atom, R¹⁴ is a hydrogen atom, R¹⁵ is a hydrogen atom, R¹⁶ is an acetyl group, X is NCH₃, and the combination of R¹, R², R⁵, R⁶, R⁷, and R⁸ is any one of the combinations shown in [Table L100] to [Table L180] (hereinafter, referred to as a compound group SX1804).

The compound (L-3), wherein R¹³ is a trifluoroacetyl group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1805).

The compound (L-3), wherein R¹³ is a hydrogen atom, R¹⁴ is a trifluoroacetyl group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1806).

The compound (L-3), wherein R¹³ is a hydrogen atom, R¹⁴ is a hydrogen atom, R¹⁵ is a trifluoroacetyl group, R¹⁶ is a hydrogen atom, X is NCH₃, and the combination of R′, R², R³, R⁶, R⁷, and R⁸ is any one of the combinations shown in [Table L100] to [Table L180] (hereinafter, referred to as a compound group SX1807).

The compound (L-3), wherein R¹³ is a hydrogen atom, R¹⁴ is a hydrogen atom, R¹⁵ is a hydrogen atom, R¹⁶ is a trifluoroacetyl group, X is NCH₃, and the combination of R¹, R², R⁵, R⁶, R⁷, and R⁸ is any one of the combinations shown in [Table L100] to [Table L180] (hereinafter, referred to as a compound group SX1808).

The compound (L-3), wherein R¹³ is a methoxycarbonyl group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1809).

The compound (L-3), wherein R¹³ is a hydrogen atom, R¹⁴ is a methoxycarbonyl group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1810).

The compound (L-3), wherein R¹³ is a hydrogen atom, R¹⁴ is a hydrogen atom, R¹⁵ is a methoxycarbonyl group, R¹⁶ is a hydrogen atom, X is NCH₃, and the combination of R′, R², R⁵, R⁶, R⁷, and R⁸ is any one of the combinations shown in [Table L100] to [Table L180] (hereinafter, referred to as a compound group SX1811).

The compound (L-3), wherein R¹³ is a hydrogen atom, R¹⁴ is a hydrogen atom, R¹⁵ is a hydrogen atom, R¹⁶ is a methoxycarbonyl group, X is NCH₃, and the combination of R¹, R², R⁵, R⁶, R⁷, and R⁸ is any one of the combinations shown in [Table L100] to [Table L180] (hereinafter, referred to as a compound group SX1812).

The compound (L-3), wherein R¹³ is a trifluoromethoxycarbonyl group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1813).

The compound (L-3), wherein R¹³ is a hydrogen atom, R¹⁴ is a trifluoromethoxycarbonyl group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1814).

The compound (L-3), wherein R¹³ is a hydrogen atom, R¹⁴ is a hydrogen atom, R¹⁵ is a trifluoromethoxycarbonyl group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1815).

The compound (L-3), wherein R¹³ is a hydrogen atom, R¹⁴ is a hydrogen atom, R¹⁵ is a hydrogen atom, R¹⁶ is a trifluoromethoxycarbonyl group, X is NCH₃, and the combination of R¹, R², R⁵, R⁶, R⁷, and R⁸ is any one of the combinations shown in [Table L100] to [Table L180] (hereinafter, referred to as a compound group SX1816).

The compound (L-3), wherein R¹³ is an acetoxy group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1817).

The compound (L-3), wherein R¹³ is a hydrogen atom, R¹⁴ is an acetoxy group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1818).

The compound (L-3), wherein R¹³ is a hydrogen atom, R¹⁴ is a hydrogen atom, R¹⁵ is an acetoxy group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1819).

The compound (L-3), wherein R¹³ is a hydrogen atom, R¹⁴ is a hydrogen atom, R¹⁵ is a hydrogen atom, R¹⁶ is an acetoxy group, X is NCH₃, and the combination of R¹, R², R⁵, R⁶, R⁷, and R⁸ is any one of the combinations shown in [Table L100] to [Table L180] (hereinafter, referred to as a compound group SX1820).

The compound (L-3), wherein R¹³ is a trifluoroacetoxy group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1821).

The compound (L-3), wherein R¹³ is a hydrogen atom, R¹⁴ is a trifluoroacetoxy group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1822).

The compound (L-3), wherein R¹³ is a hydrogen atom, R¹⁴ is a hydrogen atom, R¹⁵ is a trifluoroacetoxy group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1823).

The compound (L-3), wherein R¹³ is a hydrogen atom, R¹⁴ is a hydrogen atom, R¹⁵ is a hydrogen atom, R¹⁶ is a trifluoroacetoxy group, X is NCH₃, and the combination of R¹, R², R⁵, R⁶, R⁷, and R⁸ is any one of the combinations shown in [Table L100] to [Table L180] (hereinafter, referred to as a compound group SX1824).

The compound (L-3), wherein R¹³ is a cyano group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1825).

The compound (L-3), wherein R¹³ is a hydrogen atom, R¹⁴ is a cyano group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1826).

The compound (L-3), wherein R¹³ is a hydrogen atom, R¹⁴ is a hydrogen atom, R¹⁵ is a cyano group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1827).

The compound (L-3), wherein R¹³ is a hydrogen atom, R¹⁴ is a hydrogen atom, R¹⁵ is a hydrogen atom, R¹⁶ is a cyano group, X is NCH₃, and the combination of R¹, R², R⁵, R⁶, R⁷, and R⁸ is any one of the combinations shown in [Table L100] to [Table L180] (hereinafter, referred to as a compound group SX1828).

The compound (L-3), wherein R¹³ is a formyl group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1829).

The compound (L-3), wherein R¹³ is a hydrogen atom, R¹⁴ is a formyl group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1830).

The compound (L-3), wherein R¹³ is a hydrogen atom, R¹⁴ is a hydrogen atom, R¹⁵ is a formyl group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1831).

The compound (L-3), wherein R¹³ is a hydrogen atom, R¹⁴ is a hydrogen atom, R¹⁵ is a hydrogen atom, R¹⁶ is a formyl group, X is NCH₃, and the combination of R¹, R², R⁵, R⁶, R⁷, and R⁸ is any one of the combinations shown in [Table L100] to [Table L180] (hereinafter, referred to as a compound group SX1832).

The compound (L-3), wherein R¹³ is a carboxy group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1833).

The compound (L-3), wherein R¹³ is a hydrogen atom, R¹⁴ is a carboxy group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1834).

The compound (L-3), wherein R¹³ is a hydrogen atom, R¹⁴ is a hydrogen atom, R¹⁵ is a carboxy group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1835).

The compound (L-3), wherein R¹³ is a hydrogen atom, R¹⁴ is a hydrogen atom, R¹⁵ is a hydrogen atom, R¹⁶ is a carboxy group, X is NCH₃, and the combination of R¹, R², R⁵, R⁶, R⁷, and R⁸ is any one of the combinations shown in [Table L100] to [Table L180] (hereinafter, referred to as a compound group SX1836).

The compound (L-3), wherein R¹³ is a nitro group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1837).

The compound (L-3), wherein R¹³ is a hydrogen atom, R¹⁴ is a nitro group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1838).

The compound (L-3), wherein R¹³ is a hydrogen atom, R¹⁴ is a hydrogen atom, R¹⁵ is a nitro group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1839).

The compound (L-3), wherein R¹³ is a hydrogen atom, R¹⁴ is a hydrogen atom, R¹⁵ is a hydrogen atom, R¹⁶ is a nitro group, X is NCH₃, and the combination of R¹, R², R⁵, R⁶, R⁷, and R⁸ is any one of the combinations shown in [Table L100] to [Table L180] (hereinafter, referred to as a compound group SX1840).

The compound (L-3), wherein R¹³ is a hydroxy group, R¹⁴ is a hydrogen atom, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1841).

The compound (L-3), wherein R¹³ is a hydrogen atom, R¹⁴ is a hydroxy group, R¹⁵ is a hydrogen atom, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1842).

The compound (L-3), wherein R¹³ is a hydrogen atom, R¹⁴ is a hydrogen atom, R¹⁵ is a hydroxy group, R¹⁶ is a hydrogen atom, X is NCH₃, and the combination of R¹, R², R⁵, R⁶, R⁷, and R⁸ is any one of the combinations shown in [Table L100] to [Table L180] (hereinafter, referred to as a compound group SX1843).

The compound (L-3), wherein R¹³ is a hydrogen atom, R¹⁴ is a hydrogen atom, R¹⁵ is a hydrogen atom, R¹⁶ is a hydroxy group, X is NCH₃, and the combination of R¹, R², R⁵, R⁶, R⁷, and R⁸ is any one of the combinations shown in [Table L100] to [Table L180] (hereinafter, referred to as a compound group SX1844).

Then, formulation examples of the Compound W of the present invention will be described. Parts represent parts by weight. In addition, the Compound S of the present invention represents a compound described in the compound groups SX1 to SX1844.

### Formulation Example 1

35 parts of a mixture of polyoxyethylene alkyl ether sulfate ammonium salt and silica (weight ratio 1:1), 10 parts of any one of the Compounds S of the present invention, and 55 parts of water are mixed, and finely pulverized by wet grinding method to provide a formulation.

### Formulation Example 2

50 parts of any one of the Compounds S of the present invention, 3 parts of calcium lignosulfonate, 2 parts of sodium lauryl sulfate, and 45 parts of silica are pulverized and mixed to provide a formulation.

### Formulation Example 3

5 parts of any one of the Compounds S of the present invention, 9 parts of polyoxyethylene styrylphenyl ether, 5 parts of polyoxyethylene decyl ether (addition number of ethylene oxide: 5), 6 parts of calcium dodecylbenzene sulfonate, and 75 parts of xylene are mixed to provide a formulation.

### Formulation Example 4

2 parts of any one of the Compounds S of the present invention, 1 part of silica, 2 parts of calcium lignosulfonate, 30 parts of bentonite, and 65 parts of kaolin clay are pulverized and mixed, an appropriate amount of water is added thereto, the mixture is kneaded, granulated by a granulator, and then dried to provide a formulation.

### Formulation Example 5

10 parts of any one of the Compounds S of the present invention is mixed with a mixture of 18 parts of benzyl alcohol and 9 parts of DMSO, 6.3 parts of GERONOL (registered trademark) TE250, 2.7 parts of Ethylan (registered trademark)NS-500LQ, and 54 parts of solvent naphtha are added thereto, and the mixture is mixed to provide a formulation.

### Formulation Example 6

0.1 parts of any one of the Compounds S of the present invention and 39.9 parts of kerosene are mixed and dissolved, and placed in an aerosol container, and filled with 60 parts of liquefied petroleum gas (mixture of propane, butane, and isobutane and saturated vapor pressure: 0.47 MPa (25° C.)) to provide a formulation.

### Formulation Example 7

0.2 parts of any one of the Compounds S of the present invention, 50 parts of pyrethrum extract cake powder, 30 parts of tab powder, and 19.8 parts of wood powder are mixed, an appropriate amount of water is added thereto, and the mixture is kneaded, then subjected to an extruder to form a plate-shaped sheet, and formed into a spiral shape by a punching machine, thereby providing a formulation.

Then, the efficacy of the Compound W of the present invention against harmful arthropod is shown by Test Examples. In the following Test Examples, the test was performed at 25° C.

### Test Method 1

A test compound is formed into a formulation according to the method described in Formulation Example 1, and water containing 0.03% by volume of Sindain (registered trademark) is added thereto to prepare a dilution containing the test compound at a predetermined concentration.

A cucumber (Cucumis sativus) seedling (second true leaf stage) planted in a container is inoculated with about 30 cotton aphids (Aphis gossypii) (whole stage). One day later, the dilution is sprayed onto the seedlings at a rate of 10 mL/seedling. After 5 days, the number of surviving insects is examined, and the controlling value is determined by the following formula.

$$Controlling value ( \% ) = \{ 1 - ( Cb \times Tai ) / ( Cai \times Tb ) \} \times 100$$

wherein the symbols in the formula represent the following meanings.
Cb: number of test insects in a non-treated section
Cai: number of surviving insects in a non-treated section at the time of investigation
Tb: number of test insects in a treated section
Tai: number of surviving insects in a treated section at the time of investigation

Herein, the non-treated section means a section that is subjected to the same operation as that of the treated section except that the test compound is not used.

### Test Example 1-1

As a result of performing a test according to test method 1 using the following Compound of the present invention as a test compound at a predetermined concentration of 500 ppm, the following Compounds of the present invention all exhibited a controlling value of 90% or more.

Compound of the present invention: 50, 85, 111

### Test Method 2

A test compound is formed into a formulation according to the method described in Formulation Example 1, and water containing 0.03% by volume of Sindain (registered trademark) is added thereto to prepare a dilution containing the test compound at a predetermined concentration.

A cabbage (Brassicae oleracea) seedling (2 to 3 true leaf stage) planted in a container is sprayed with the dilution at a rate of 20 mL/seedling. Thereafter, the foliage portion of the seedling is cut out and placed in a container on which filter paper is laid. Thereto are released five second instar larvae of common cutworm (Spodoptera litura). After 5 days, the number of surviving insects is counted, and the mortality is determined from the following equation.

$$Mortality ( \% ) = ( 1 - number of surviving insects / 5 ) \times 100$$

### Test Example 2-1

As a result of performing a test according to test method 2 using the following Compound of the present invention as a test compound at a predetermined concentration of 500 ppm, the following Compounds of the present invention all exhibited a mortality of 80% or more.

Compound of the present invention: 1, 3, 5, 6, 7, 8, 9, 10, 11, 13, 15, 16, 17, 20, 21, 22, 23, 25, 27, 28, 30, 31, 32, 34, 36, 37, 38, 39, 40, 41, 42, 43, 44, 45, 46, 47, 49, 50, 51, 52, 53, 54, 55, 56, 57, 58, 59, 60, 61, 62, 63, 64, 65, 66, 67, 68, 69, 70, 71, 72, 74, 75, 76, 77, 78, 80, 81, 83, 84, 85, 86, 87, 88, 90, 91, 92, 94, 95, 96, 98, 99, 100, 102, 103, 104, 105, 108, 109, 110, 111, 113, 114, 115, 116, 119, 120, 121, 122, 123, 124, 125, 126, 127, 128, 129, 131, 132, 133, 135, 136, 138, 140, 141, 142, 143, 144, 145, 146, 147, 148, 149, 150, 151, 152, 156, 157, 158, 159, 160, 161, 162, 164, 165, 166, 167, 168, 169, 170, 173, 174, 175, 176

### Test Method 3

A test compound is formed into a formulation according to the method described in Formulation Example 1, and water containing 0.03% by volume of Sindain (registered trademark) is added thereto to prepare a dilution containing the test compound at a predetermined concentration.

A cabbage (Brassicae oleracea) seedling (2 to 3 true leaf stage) planted in a container is sprayed with the dilution at a rate of 20 mL/seedling. Thereafter, the foliage portion of the seedling is cut out and placed in a container on which filter paper is laid. Thereto are released five second instar larvae of diamondback moth (Plutella xylostella). After 5 days, the number of surviving insects is counted, and the mortality is determined from the following equation.

$$Mortality ( \% ) = ( 1 - number of surviving insects / 5 ) \times 100$$

### Test Example 3-1

As a result of performing a test according to test method 3 using the following Compound of the present invention as a test compound at a predetermined concentration of 500 ppm, the following Compounds of the present invention all exhibited a mortality of 80% or more.

Compound of the present invention: 1, 2, 3, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 85, 86, 87, 88, 89, 90, 91, 92, 94, 95, 96, 98, 99, 100, 101, 102, 103, 104, 105, 107, 108, 109, 110, 111, 112, 113, 114, 115, 116, 118, 119, 120, 121, 123, 124, 125, 126, 127, 128, 129, 130, 131, 132, 133, 134, 135, 136, 137, 138, 139, 140, 141, 142, 143, 144, 145, 146, 147, 148, 150, 151, 152, 154, 155, 156, 157, 158, 159, 160, 161, 162, 163, 164, 165, 168, 169, 170, 171, 172, 173, 174, 175, 176

### Test Method 4

Each 1 mg of the test compound is dissolved in 50 μL of a mixed solution of polyoxyethylene sorbitan monococoate and acetone (5:95 (volume ratio)). Water containing 0.03% by volume of Sindain (registered trademark) is added thereto to prepare a dilution containing the test compound at a predetermined concentration.

Young seedlings of maize (Zea mays) are immersed in the dilution for 30 seconds. Thereafter, the two fruits are placed in a petri dish (90 mm diameter), and thereto are released 10 second instar larvae of Western corn rootworms (Diabrotica virgifera virgifera). After 5 days, the number of dead insects is counted, and the mortality is determined from the following equation.

Mortality(%)=(number of dead insects/10)×100

### Test Example 4-1

As a result of performing a test according to test method 4 using the following Compound of the present invention as a test compound at a predetermined concentration of 200 ppm, the following Compounds of the present invention exhibited a mortality of 90% or more.

Compound of the present invention: 8, 27, 41, 50, 100, 114, 116

### Test Method 5

An acetone solution prepared such that the amount of the test compound is 800 ppm is poured into a container having an internal volume of 50 mb, and the inner surface of the container is uniformly coated such that the amount of the test compound is 40 mg/m², and then dried.

Five adult male German cockroaches (Blattella germanica) are placed in the container, and the lid is closed. After a lapse of a predetermined time, the state of German cockroaches (Blattella germanica) is examined, and the mortality is determined. The mortality is calculated by the following equation.

$$Mortality ( \% ) = ( number of dead insects / number of tested insects ) \times 100$$

### Test Example 5-1

As a result of performing a test according to test method 5 using the following Compound of the present invention as a test compound at a predetermined time of one hour, the following Compound of the present inventions all exhibited a mortality of 80% or more.

Compound of the present invention: 1, 2, 3, 4, 6, 7, 8, 9, 10, 11, 13, 16, 17, 18, 19, 20, 21, 22, 23, 26, 27, 28, 29, 30, 31, 32, 34, 38, 39, 41, 42, 43, 44, 45, 46, 47, 48, 49, 50, 51, 52, 53, 54, 55, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 90, 91, 94, 96, 97, 98, 99, 100, 101, 102, 103, 104, 105, 108, 109, 110, 111, 113, 115, 119, 120, 121, 122, 123, 124, 138, 142, 145, 146, 147, 148, 149, 150, 151, 156, 158, 159, 164, 174, 175, 176

### Test Example 5-2

As a result of performing a test according to test method 5 using the following Compound of the present invention as a test compound at a predetermined time of one day, the following Compounds of the present invention all exhibited a mortality of 80% or more.

Compound of the present invention: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 36, 37, 38, 39, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 90, 91, 92, 94, 96, 97, 98, 99, 100, 101, 102, 103, 104, 105, 106, 108, 109, 110, 111, 112, 113, 114, 115, 116, 117, 118, 119, 120, 121, 122, 123, 124, 125, 126, 128, 129, 130, 131, 132, 133, 135, 136, 137, 138, 139, 142, 143, 144, 145, 146, 147, 148, 149, 150, 151, 152, 156, 157, 158, 159, 160, 161, 164, 165, 166, 167, 168, 169, 170, 171; 173, 174, 175, 176

### Test Example 5-3

As a result of performing a test according to test method 5 using the following Compound of the present invention as a test compound at a predetermined time of three days, the following Compounds of the present invention all exhibited a mortality of 80% or more.

Compound of the present invention: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 90, 91, 92, 94, 96, 97, 98, 99, 100, 101, 102, 103, 104, 105, 106, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 142, 143, 144, 145, 146, 147, 148, 149, 150, 151, 152, 156, 157, 158, 159, 160, 161, 163, 164, 165, 166, 167, 168, 169, 170, 171, 173, 174, 175, 176

### Test Method 6

An acetone solution prepared such that the amount of the test compound is 2000 ppm is poured into a container having an internal volume of 20 mb, and the inner surface of the container is uniformly coated such that the amount of the test compound is 100 mg/m², and then dried.

Five nymphs of Haemaphysalis longicornis are placed in the container, and the lid is closed. After a lapse of a predetermined time, the state of Haemaphysalis longicornis is examined to determine the mortality. The mortality is calculated by the following equation.

$$Mortality ( \% ) = ( number of dead insects / number of tested insects ) \times 100$$

### Test Example 6-1

As a result of performing a test according to test method 6 using the following Compound of the present invention as a test compound at a predetermined time of two days, the following Compounds of the present invention all exhibited a mortality of 80% or more.

Compound of the present invention: 1, 3, 4, 5, 6, 8, 9, 10, 11, 12, 13, 14, 16, 17, 19, 20, 21, 22, 23, 26, 27, 28, 29, 30, 31, 32, 33, 34, 37, 38, 39, 40, 41, 43, 44, 45, 47, 48, 49, 50, 51, 52, 53, 54, 55, 57, 58, 59, 60, 61, 62, 63, 64, 65, 66, 68, 69, 71, 72, 73, 74, 75, 76, 77, 78, 79, 80, 81, 82, 83, 84, 85, 86, 87, 88, 90, 91, 92, 94, 95, 96, 98, 99, 100, 101, 102, 103, 104, 105, 107, 108, 109, 110, 111, 113, 114, 115, 116, 119, 120, 121, 122, 124, 125, 126, 128, 130, 131, 132, 133, 135, 136, 138, 139, 140, 141, 142, 143, 144, 145, 146, 148, 149, 156, 157, 158, 159, 160, 161, 164

### Test Method 7

An acetone solution prepared such that the amount of the test compound is 800 ppm is poured into a container having an internal volume of 20 mb, and the inner surface of the container is uniformly coated such that the amount of the test compound is 40 mg/m², and then dried.

Five adult female house flies (Musca domestica) are placed in the container, and the lid is closed. After a lapse of a predetermined time, the state of house flies (Musca domestica) is examined, and the mortality is determined. The mortality is calculated by the following equation.

$$Mortality ( \% ) = ( number of dead insects / number of tested insects ) \times 100$$

### Test Example 7-1

As a result of performing a test according to test method 7 using the following Compound of the present invention as a test compound at a predetermined time of one hour, the following Compounds of the present invention all exhibited a mortality of 80% or more.

Compound of the present invention: 1, 2, 3, 4, 5, 7, 8, 9, 10, 11, 13, 14, 15, 16, 17, 18, 19, 20, 21, 22, 23, 24, 25, 26, 27, 28, 29, 30, 31, 32, 33, 34, 36, 37, 38, 39, 40, 41, 42, 43, 44, 45, 46, 47, 48, 49, 50, 51, 52, 53, 54, 55, 57, 58, 59, 60, 61, 62, 63, 64, 65, 66, 67, 70, 71, 72, 73, 74, 75, 76, 77, 78, 79, 80, 81, 82, 83, 84, 85, 86, 87, 88, 90, 91, 92, 94, 95, 96, 97, 98, 99, 100, 101, 102, 103, 104, 105, 106, 107, 108, 109, 110, 111, 113, 114, 115, 116, 117, 118, 120, 121, 122, 123, 124, 125, 126, 128, 129, 131, 135, 136, 137, 138, 139, 140, 141, 142, 143, 144, 145, 146, 147, 148, 149, 150, 151, 152, 156, 157, 158, 159, 160, 161, 166, 167, 168, 169, 170, 173, 174, 175, 176

### Test Example 7-2

As a result of performing a test according to test method 7 using the following Compound of the present invention as a test compound at a predetermined time of one day, the following Compounds of the present invention all exhibited a mortality of 80% or more.

Compound of the present invention: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 90, 91, 92, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 155, 156, 157, 158, 159, 160, 161, 162, 164, 165, 166, 167, 168, 169, 170, 173, 174, 175, 176

### Test Method 8

An acetone solution prepared such that the amount of the test compound is 200 ppm is poured into a container having an internal volume of 20 mL, and the inner surface of the container is uniformly coated such that the amount of the test compound is 10 mg/m², and then dried, Five adult female common house mosquitos (Culex pipiens pallens) are placed in the container, and the lid is closed. After a lapse of a predetermined time, the state of common house mosquitos (Culex pipiens pallens) is examined, and the mortality is determined. The mortality is calculated by the following equation.

$$Mortality ( \% ) = ( number of dead insects / number of tested insects ) \times 100$$

### Test Example 8-1

As a result of performing a test according to test method 8 using the following Compound of the present invention as a test compound at a predetermined time of one hour, the following Compounds of the present invention all exhibited a mortality of 80% or more.

Compound of the present invention: 1, 2, 3, 4, 5, 6, 7, 8, 9, 10, 11, 13, 14, 15, 16, 17, 18, 19, 20, 21, 22, 23, 25, 26, 27, 28, 29, 30, 31, 32, 33, 34, 36, 37, 38, 39, 40, 41, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 79, 80, 81, 83, 84, 85, 86, 87, 88, 90, 91, 94, 95, 96, 97, 98, 99, 100, 101, 102, 103, 104, 105, 106, 108, 109, 110, 111, 113, 114, 115, 116, 117, 119, 120, 121, 122, 123, 124, 128, 129, 131, 136, 138, 139, 140, 142, 143, 144, 145, 146, 147, 148, 149, 150, 151, 156, 157, 158, 159, 160, 161, 165, 166, 167, 168, 169, 170, 173, 174, 175, 176

### Test Example 8-2

As a result of performing a test according to test method 8 using the following Compound of the present invention as a test compound at a predetermined time of one day, the following Compounds of the present invention all exhibited a mortality of 80% or more.

Compound of the present invention: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 156, 157, 158, 159, 160, 161, 162, 164, 165, 166, 167, 168, 169, 170, 171, 173, 174, 175, 176

### Test Method 9

An acetone solution prepared such that the amount of the test compound is 50 ppm is poured into a container having an internal volume of 20 mL, and the inner surface of the container is uniformly coated such that the amount of the test compound is 2.5 mg/m², and then dried.

10 adult female Pharaoh ants (Monomorium pharaonis) are placed in the container, and the lid is closed. After a lapse of a predetermined time, the state of Pharaoh ants (Monomorium pharaonis) is examined, and the mortality is determined. The mortality is calculated by the following equation.

$$Mortality ( \% ) = ( number of dead insects / number of tested insects ) \times 100$$

### Test Example 9-1

As a result of performing a test according to test method 9 using the following Compound of the present invention as a test compound at a predetermined time of one hour, the following Compounds of the present invention all exhibited a mortality of 70% or more.

Compound of the present invention: 2, 3, 4, 8, 9, 17, 18, 19, 20, 21, 22, 23, 24, 25, 26, 28, 29, 30, 31, 32, 33, 34, 36, 38, 39, 41, 43, 44, 45, 46, 47, 48, 49, 50, 51, 52, 53, 54, 55, 56, 57, 58, 59, 60, 61, 62, 63, 64, 65, 66, 67, 68, 69, 72, 73, 74, 77, 78, 79, 80, 81, 83, 85, 87, 88, 90, 91, 92, 95, 96, 97, 98, 99, 100, 101, 102, 103, 108, 109, 110, 111, 113, 114, 115, 116, 117, 119, 120, 121, 122, 124, 125, 126, 128, 130, 131, 132, 133, 134, 135, 136, 138, 139, 140, 141, 142, 143, 144, 145, 146, 147, 148, 149, 150, 151, 152, 156, 157, 158, 159, 160, 161, 164, 166, 167, 168, 169, 170, 171, 173, 174, 175, 176

### Test Example 9-2

As a result of performing a test according to test method 9 using the following Compound of the present invention as a test compound at a predetermined time of one day, the following Compounds of the present invention all exhibited a mortality of 70% or more.

Compound of the present invention: 1, 2, 3, 4, 5, 6, 7, 8, 9, 10, 11, 13, 15, 16, 17, 18, 19, 20, 21, 22, 23, 24, 25, 26, 27, 28, 29, 30, 31, 32, 33, 34, 35, 36, 37, 38, 39, 41, 42, 43, 44, 45, 46, 47, 48, 49, 50, 51, 52, 53, 54, 55, 56, 57, 58, 59, 60, 61, 71, 72, 73, 74, 75, 76, 77, 78, 79, 80, 81, 83, 84, 85, 86, 87, 88, 90, 91, 92, 94, 95, 96, 97, 98, 99, 100, 101, 102, 103, 104, 105, 107, 108, 109, 110, 111, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 156, 157, 158, 159, 160, 161, 162, 164, 165, 166, 167, 168, 169, 170, 171, 173, 174, 175, 176

### Test Method 10

A test compound is formed into a formulation according to the method described in Formulation Example 5, and water containing 0.03% by volume of Sindain (registered trademark) is added thereto to prepare a dilution containing the test compound at a predetermined concentration.

A cucumber (Cucumis sativus) seedling (second true leaf stage) planted in a container is sprayed with the dilution at a rate of 10 mb/seedling. Thereafter, the first true leaf is cut out and stored in a container, and approximately 20 larvae of western flower thrips (Frankliniella occidentalis) are released into the container. After 7 days, the number of surviving insects is examined, and the mortality is determined by the following equation.

$$Mortality ( \% ) = \{ 1 - number of surviving insects / 20 \} \times 100$$

### Test Example 10-1

As a result of performing a test according to test method 10 using the following Compound of the present invention as a test compound at a predetermined concentration of 200 ppm, the following Compounds of the present invention all exhibited a mortality of 90% or more.

Compound of the present invention: 3, 7, 8, 9, 13, 15, 16, 17, 18, 19, 20, 25, 29, 37, 38, 39, 42, 44, 45, 46, 47, 48, 49, 50, 51, 54, 55, 57, 58, 60, 62, 63, 64, 65, 66, 69, 71, 72, 75, 76, 77, 78, 80, 81, 83, 84, 85, 88, 90, 91, 94, 95, 96, 99, 100, 102, 103, 104, 105, 107, 108, 109, 110, 111, 113, 114, 115, 116, 119, 120, 122, 124, 126, 129, 131, 136, 138, 139, 140, 141, 142, 143, 144, 145, 146, 149, 150, 151, 152, 156, 157, 158, 159, 160, 161, 167, 168, 170, 173, 174, 175

### Test Method 11

A test compound is formed into a formulation according to the method described in Formulation Example 5, and water containing 0.03% by volume of Sindain (registered trademark) is added thereto to prepare a dilution containing the test compound at a predetermined concentration.

A cabbage (Brassicae oleracea) seedling (3 to 4 true leaf stage) planted in a container is sprayed with the dilution at a rate of 20 mL/seedling. Thereafter, 10 third instar larvae of common cutworm (Spodoptera litura) are released. After 6 days, the number of surviving insects is counted, and the mortality is determined from the following equation.

Mortality(%)=(1−number of surviving insects/10)×100

### Test Example 11-1

As a result of performing a test according to test method 11 using the following Compound of the present invention as a test compound at a predetermined concentration of 200 ppm, the following Compounds of the present invention all exhibited a mortality of 80% or more.

Compound of the present invention: 1, 3, 5, 6, 7, 8, 9, 10, 13, 15, 16, 17, 20, 22, 27, 30, 32, 34, 36, 37, 38, 39, 42, 43, 44, 45, 46, 47, 49, 50, 51, 54, 55, 56, 57, 58, 59, 60, 62, 63, 64, 65, 66, 68, 69, 70, 71, 75, 75, 78, 80, 81, 83, 84, 85, 86, 87, 88, 90, 91, 92, 94, 95, 96, 98, 99, 100, 102, 103, 104, 105, 108, 109, 110, 111, 113, 114, 115, 116, 119, 120, 121, 122, 124, 126, 128, 129, 131, 132, 133, 135, 136, 138, 139, 140, 141, 142, 143, 144, 145, 146, 147, 148, 149, 150, 151, 152, 156, 157, 158, 159, 160, 161, 166, 167, 168, 169, 170, 173, 174, 175, 176

### Test Method 12

A test compound is formed into a formulation according to the method described in Formulation Example 5, and water containing 0.03% by volume of Sindain (registered trademark) is added thereto to prepare a dilution containing the test compound at a predetermined concentration.

A cabbage (Brassicae oleracea) seedling (3 to 4 true leaf stage) planted in a container is sprayed with the dilution at a rate of 20 mL/seedling. Thereafter, 10 third instar larvae of diamondback moth (Plutella xylostella) are released. After 5 days, the number of surviving insects is counted, and the mortality is determined from the following equation.

$$Mortality ( \% ) = ( 1 - number of surviving insects / 10 ) \times 100$$

### Test Example 12-1

As a result of performing a test according to test method 12 using the following Compound of the present invention as a test compound at a predetermined concentration of 200 ppm, the following Compounds of the present invention all exhibited a mortality of 80% or more.

Compound of the present invention: 1, 3, 5, 6, 7, 8, 9, 10, 13, 15, 16, 17, 18, 19, 20, 22, 23, 25, 27, 28, 29, 30, 31, 32, 33, 34, 35, 36, 37, 38, 39, 40, 41, 42, 43, 44, 45, 46, 47, 48, 49, 50, 51, 52, 54, 55, 56, 57, 58, 59, 60, 61, 62, 63, 64, 65, 66, 67, 68, 69, 70, 71, 72, 73, 74, 75, 81, 83, 85, 86, 87, 88, 89, 90, 91, 92, 94, 95, 96, 98, 99, 100, 101, 102, 103, 104, 105, 107, 108, 109, 110, 111, 112, 113, 114, 115, 116, 118, 119, 120, 121, 122, 124, 125, 126, 127, 128, 129, 130, 131, 132, 133, 134, 135, 136, 137, 138, 139, 140, 141, 142, 143, 144, 145, 146, 147, 148, 149, 150, 151, 152, 155, 156, 157, 158, 159, 160, 161, 162, 163, 164, 168, 169, 170, 171, 172, 173, 174, 175, 176

### Test Method 13

A test compound is formed into a formulation according to the method described in Formulation Example 5, and water containing 0.03% by volume of Sindain (registered trademark) is added thereto to prepare a dilution containing the test compound at a predetermined concentration.

A cucumber (Cucumis sativus) seedling (second true leaf stage) planted in a container is inoculated with about 30 cotton aphids (Aphis gossypii) (whole stage). One day later, the dilution is sprayed onto the seedlings at a rate of 10 mL/seedling. After 5 days, the number of surviving insects is examined, and the controlling value is determined by the following formula.

$$Controlling value ( \% ) = \{ 1 - ( Cb \times Tai ) / ( Cai \times Tb ) \} \times 100$$

wherein the symbols in the formula represent the following meanings.
Cb: number of test insects in a non-treated section
Cai: number of surviving insects in a non-treated section at the time of investigation

Tb: number of test insects in a treated section Tai: number of surviving insects in a treated section at the time of investigation Herein, the non-treated section means a section that is subjected to the same operation as that of the treated section except that the test compound is not used.

### Test Example 13-1

As a result of performing a test according to test method 13 using the following Compound of the present invention as a test compound at a predetermined concentration of 200 ppm, the following Compounds of the present invention all exhibited a controlling value of 90% or more.

Compound of the present invention: 50, 85

### Test Method 14

The test compound is formulated according to the method described in Formulation Example 1, and water is added thereto to prepare a dilution containing the test compound at a predetermined concentration.

30 last-instar larvae of common house mosquito (Culex pipiens pallens) are released into the dilution, and the state of the larvae of common house mosquito (Culex pipiens pallens) is examined one day later to determine the mortality. The mortality is calculated by the following equation.

$$Mortality ( \% ) = ( number of dead insects / number of tested insects ) \times 100$$

### Test Example 14-1

As a result of performing a test according to test method 14 using the following Compound of the present invention as a test compound at a predetermined concentration of 3.5 ppm, the following Compounds of the present invention all exhibited a mortality of 91% or more.

Compound of the present invention: 1, 2, 3, 5, 6, 7, 8, 9, 10, 11, 13, 15, 16, 17, 18, 19, 20, 25, 26, 27, 28, 29, 30, 31, 32, 33, 34, 35, 36, 37, 38, 39, 40, 45, 47, 48, 49, 50, 51, 54, 55, 56, 57, 59, 61, 62, 63, 65, 66, 67, 68, 69, 70, 74, 75, 78, 82, 83, 84, 85, 86, 87, 88, 90, 91, 92, 93, 94, 95, 96, 97, 98, 99, 100, 102, 103, 104, 105, 107, 108, 109, 110, 111, 113, 114, 116, 118, 122, 124, 125, 126, 128, 129, 130, 131, 132, 133, 135, 136, 138, 140, 141, 142, 143, 144, 145, 146, 149, 150, 151, 152, 155, 156, 157, 158, 159, 160, 161, 164, 166, 167, 168, 169, 170, 171, 172, 173, 174, 175, 176

### Test Method 15

A test compound is formed into a formulation according to the method described in Formulation Example 1, and water containing 0.03% by volume of Sindain (registered trademark) is added thereto to prepare a dilution containing the test compound at a predetermined concentration.

A rice (Oryza sativa) seedling (second leaf stage) planted in a container is sprayed with the dilution at a rate of 10 mL/seedling. Thereafter, 20 third-instar nymphs of brown planthopper (Nilaparvata lugens) are released. After 6 days, the number of surviving insects is examined, and the mortality is determined by the following equation.

$$Mortality ( \% ) = \{ 1 - number of surviving insects / 20 \} \times 100$$

### Test Example 15-1

As a result of performing a test according to Test Example 15 using the following Compound of the present invention as a test compound at a predetermined concentration of 500 ppm, the following Compounds of the present invention all exhibited a mortality of 90% or more.

Compound of the present invention: 2, 3, 8, 13, 17, 21, 23, 26, 27, 28, 29, 31, 37, 38, 41, 42, 44, 45, 46, 47, 48, 49, 51, 52, 53, 54, 55, 58, 59, 60, 61, 62, 63, 64, 65, 66, 69, 72, 76, 77, 78, 81, 85, 88, 90, 91, 94, 96, 98, 99, 100, 102, 103, 109, 110, 111, 113, 114, 115, 116, 119, 121, 122, 123, 124, 125, 126, 131, 133, 138, 141, 142, 145, 146, 147, 148, 149, 150, 151, 152, 156, 157, 158, 159, 166, 167, 168

### Test Method 16

A test compound is formed into a formulation according to the method described in Formulation Example 5, and water containing 0.03% by volume of Sindain (registered trademark) is added thereto to prepare a dilution containing the test compound at a predetermined concentration.

A rice (Oryza sativa) seedling (second leaf stage) planted in a container is sprayed with the dilution at a rate of 10 mL/seedling. Thereafter, 20 third-instar nymphs of the brown planthopper (Nilaparvata lugens) are released. After 6 days, the number of surviving insects is examined, and the mortality is determined by the following equation.

$$Mortality ( \% ) = \{ 1 - number of surviving insects / 20 \} \times 100$$

### Test Example 16-1

As a result of performing a test according to Test Example 16 using the following Compound of the present invention as a test compound at a predetermined concentration of 200 ppm, the following Compounds of the present invention all exhibited a mortality of 90% or more.

Compound of the present invention: 2, 9, 13, 16, 41, 42, 44, 46, 47, 48, 50, 51, 52, 55, 57, 58, 63, 64, 65, 66, 76, 77, 78, 81, 83, 88, 90, 98, 100, 102, 103, 109, 110, 111, 113, 114, 115, 116, 119, 121, 122, 131, 132, 133, 139, 141, 142, 143, 144, 145, 146, 147, 148, 149, 150, 156, 158, 159, 160, 166, 167, 168, 170

### Test Method 17

1 mg of the Compound W of the present invention is dissolved in 10 μL of a mixed solution of xylene:DMF:surfactant=4:4:1 (volume ratio), and diluted with water containing 0.028 by volume of an adjuvant to prepare a dilution A containing the Compound W of the present invention at a predetermined concentration.

1 mg of the present component is dissolved in 10 μL of a mixed solution of xylene:DMF:surfactant=4:4:1 (volume ratio), and diluted with water containing 0.02% by volume of an adjuvant to prepare a dilution B containing the present component at a predetermined concentration.

The dilution A and dilution B are mixed to provide a dilution C.

Leaf pieces of cucumber cotyledons (length 1.5 cm) are accommodated in each well of a 24 well microplate, 2 apterous adults and 8 larvae of cotton aphids (Aphis gossypii) are released per well, and 20 μL of the dilution C is sprayed per well. This is defined as a treated section.

A well to which 20 μl of water containing 0.02% by volume of an adjuvant is sprayed instead of the dilution C is defined as a non-treated section.

After the dilution C is dried, the upper portion of the microplate is covered with a film sheet. After 5 days, the number of surviving insects in each well is examined.

The controlling value is calculated from the following equation.

$$Controlling value ( \% ) = \{ 1 - ( Tai ) / ( Cai ) \} \times 100$$

wherein the symbols in the equation represent the following meanings.
Cai: number of surviving insects in a non-treated section at the time of investigation
Tai: number of surviving insects in a treated section at the time of investigation

Specific dilutions C in which effects can be confirmed by test method 17 are shown in the following 1) to 5).
1) Dilution C having a concentration of the Compound W of the present invention of 200 ppm and a concentration of the present component of 2000 ppm in the combination shown in the list A. In the list A, Comp X means any one compound selected from the compound groups SX1 to SX1844.
List A: Comp X+clothianidin; Comp X+thiamethoxam; Comp X+imidacloprid; Comp X+thiacloprid; Comp X+flupyradifuron; Comp X+sulfoxaflor; Comp X+triflumezopyrime; Comp X+dicloromezotiaz; Comp X+beta cyfluthrin; Comp X+tefluthrin; Comp X+fipronil; Comp X+chlorantraniliprole; Comp X+cyantraniliprole; Comp X+tetraniliprole; Comp X+thiodicarb; Comp X+carbofuran; Comp X+fluxamethamide; Comp X+afoxolanel; Comp X+fluraranel; Comp X+brofuranilide; Comp X+abamectin; Comp X+fluopyram; Comp X+fluenesulfone; Comp X+fluazaindolidine; Comp X+thioxazafen; Comp X+flupyrimine; Comp X+mycorrhizal fungi; Comp X+Bradyrhizobium japonicum TA-11 strain; Comp X+Bacillus firmus; Comp X+Bacillus firmus I-1582 strain; Comp X+Bacillus amyloliquefaciens; Comp X+Bacillus amyloliquefaciens FZB42 strain; Comp X+pathuria nishizawae; Comp X+pathuria nishizawae Pn1 strain; Comp X+pasteuria penetrans; Comp X+tebuconazole; Comp X+prothioconazole; Comp X+metconazole; Comp X+ipconazole; Comp X+triticonazole; Comp X+difenoconazole; Comp X+imazalyl; Comp X+triazimenol; Comp X+tetraconazole; Comp X+flutriafol; Comp X+mandestrobin; Comp X+azoxystrobin; Comp X+pyraclostrobin; Comp X+trifloxystrobin; Comp X+fluoxastrobin; Comp X+picoxystrobin; Comp X+phenamidone; Comp X+metalaxyl; Comp X+metalaxyl M; Comp X+fludioxonil; Comp X+sedaxane; Comp X+penflufen; Comp X+fluxapyroxad; Comp X+benzobindiflupyr; Comp X+boscalid; Comp X+carboxine; Comp X+penthiopyrad; Comp X+flutolanyl; Comp X+captan; Comp X+thiuram; Comp X+tolclofos-methyl; Comp X+thiabendazole; Comp X+etaboxam; Comp X+mancozeb; Comp X+picarbutrazox; Comp X+oxathiapiproline; Comp X+silthiopham; and Comp X+impilfluxam.
2) Dilution C having a concentration of the Compound W of the present invention of 200 ppm and a concentration of the present component of 200 ppm in the combination shown in the list A.
3) Dilution C having a concentration of the Compound W of the present invention of 500 ppm and a concentration of the present component of 50 ppm in the combination shown in the list A.
4) Dilution C having a concentration of the Compound W of the present invention of 500 ppm and a concentration of the present component of 5 ppm in the combination shown in the list A.
5) Dilution C having a concentration of the Compound W of the present invention of 500 ppm and a concentration of the present component of 0.5 ppm in the combination shown in the list A.

### INDUSTRIAL APPLICABILITY

The Compound W of the present invention exhibits an excellent control effect on harmful arthropod.

1. A compound represented by formula (I) 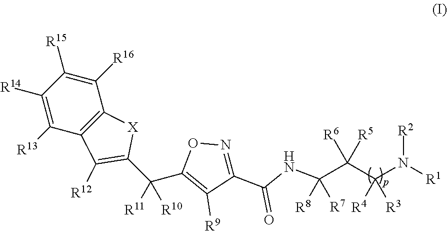
[wherein:
R¹ and R² are identical to or different from each other, and each represent a C1-C6 chain hydrocarbon group optionally having one or more substituents selected from Group A¹, a C1-C3 alkoxy group optionally having one or more halogen atoms, a benzyl group optionally having one or more substituents selected from Group B, a C1-C3 alkylcarbonyl group optionally having one or more halogen atoms, a C1-C3 alkoxycarbonyl group optionally having one or more halogen atoms, a C1-C3 alkylsulfonyl group optionally having one or more halogen atoms, or a hydrogen atom;
R¹ and R² may be combined to form —(CR¹⁷R¹⁸)— (CR¹⁹R²⁰)_{b}—(CR²⁵R²⁶)—, or —(CR²⁷R²⁸)—(CR²⁹R³⁰)—Y—(CR³¹R³²)—(CR³³R³⁴)—;
b represents 0, 1, 2, or 3;
Y represents an oxygen atom, a sulfur atom, or NR³⁵;
R¹⁷, R¹⁸, R¹⁹, R²⁰, R²⁵, R²⁶, R²⁷, R²⁸, R²⁹, R³⁰, R³¹, R³², R³³, R³⁴, and R³⁵ are identical to or different from each other, and each represent a C1-C4 alkyl group optionally having one or more halogen atoms, or a hydrogen atom;
when b is 2 or 3, R¹⁹ and R²⁰ of 2 or 3 may be each independently identical to or different from each other;
p represents 0, 1, 2, 3, 4, 5, or 6;
R³, R⁴, R⁵, R⁶, R⁷, and R⁸ are identical to or different from each other, and each represent a C1-C6 alkyl group optionally having one or more halogen atoms, or a hydrogen atom;
R³ and R⁴ may be combined to form —(CR³⁶R³⁷)—(CR³⁸R³⁹)ₑ—(CR⁴⁰R⁴¹)—, or —(CR⁴²R⁴³)ₕ—Z¹—(CR⁴⁴R⁴⁵)₁—;
R⁵ and R⁶ may be combined to form —(CR⁴⁶R⁴⁷)—(CR⁴⁸R⁴⁹)_{f}—(CR⁵⁰R⁵¹)—, or —(CR⁵²R⁵³)ⱼ—Z²—(CR⁵⁴R⁵⁵)ₖ—;
R⁷ and R⁸ may be combined to form —(CR⁵⁶R⁵⁷)—(CR⁵⁸R⁵⁹)_{g}—(CR⁶⁰R⁶¹)—, or —(CR⁶²R⁶³)ₘ—Z³—(CR⁶⁴R⁶⁵)ₙ—;
when p is 2, 3, 4, 5, or 6, R³ and R⁴ of 2, 3, 4, 5, or 6 may be each independently identical to or different from each other;
e, f, and g each independently represent 0, 1, 2, or 3;
h, i, j, k, m, and n each independently represent 1 or 2;
Z¹, Z², and Z³ are identical to or different from each other, and each represent an oxygen atom, a sulfur atom, or NR⁷⁰;
R³⁶, R³⁷, R³², R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, and R⁷⁰ are identical to or different from each other, and each represent a C1-C4 alkyl group optionally having one or more halogen atoms, or a hydrogen atom;
when e, f, or g is 2 or 3, R³⁸, R³⁹, R⁴⁸, R⁴⁹, R⁵⁸, and R⁵⁹ of 2 or 3 may be each independently identical to or different from each other;
when h, i, j, k, m, or nis 2, R⁴², R⁴³, R⁴⁴, R⁴⁵, R⁵², R⁵³, R⁵⁴, R⁵⁵, R⁶², R⁶³, R⁶⁴, and R⁶⁵ of 2 may be each independently identical to or different from each other;
a plurality of R⁷⁰ (s) may be each independently identical to or different from each other;
R⁹ represents a C1-C3 chain hydrocarbon group optionally having one or more substituents selected from Group D, a cyano group, a formyl group, a carboxy group, a (C1-C4 alkoxy) carbonyl group wherein the C1-C4 alkoxy group optionally has one or more halogen atoms, a carbamoyl group, a halogen atom, or a hydrogen atom;
R¹⁰ and R¹¹ are identical to or different from each other, and each represent a C1-C4 alkyl group optionally having one or more halogen atoms, a halogen atom, or a hydrogen atom;
R¹², R¹³, R¹⁴, R¹⁵, and R¹⁶ are identical to or different from each other, and each represent a C1-C4 chain hydrocarbon group optionally having one or more substituents selected from Group E, a C1-C4 alkoxy group optionally having one or more substituents selected from Group E, a C3-C6 alicyclic hydrocarbon group optionally having one or more substituents selected from Group E, a phenyl group optionally having one or more substituents selected from Group B, a (C1-C4 alkyl) carbonyl group wherein the C1-C4 alkyl group optionally has one or more halogen atoms, a (C1-C4 alkoxy) carbonyl group wherein the C1-C4 alkoxy group optionally has one or more halogen atoms, a (C1-C4 alkyl) carbonyloxy group wherein the C1-C4 alkyl group optionally has one or more halogen atoms, a cyano group, a formyl group, a carboxy group, a nitro group, a hydroxy group, a halogen atom, or a hydrogen atom;
R¹³ and R¹⁴ may be combined to form —O—(CR⁷⁶R⁷⁷)—O—;
R¹⁴ and R¹⁵ may be combined to form —O—(CR⁷⁸R⁷⁹)—O—;
R¹⁵ and R¹⁶ may be combined to form —O—(CR⁸⁰R⁸¹)—O—;
R⁷⁵, R⁷⁷, R⁷⁸, R⁷⁹, R⁸⁰, and R⁸¹ are identical to or different from each other, and each represent a C1-C4 alkyl group optionally having one or more halogen atoms, a halogen atom, or a hydrogen atom;
X represents an oxygen atom, a sulfur atom, or NR⁷¹, and
R⁷¹ represents a C1-C4 alkyl group optionally having one or more halogen atoms, a C1-C4 alkoxycarbonyl group optionally having one or more halogen atoms, or a hydrogen atom;
Group A¹: a group consisting of a C3-C6 alicyclic hydrocarbon group optionally having one or more substituents selected from Group E, a C1-C4 alkoxy group optionally having one or more halogen atoms, a C1-C4 alkylcarbonyl group optionally having one or more halogen atoms, a C1-C4 alkoxycarbonyl group optionally having one or more halogen atoms, a C1-C4 alkylsulfonyl group optionally having one or more halogen atoms, a pyridyl group, a furyl group, a thienyl group, an isoxazolyl group, a pyridazinyl group, a pyrimidinyl group, a pyrazinyl group {the pyridyl group, the furyl group, the thienyl group, the isoxazolyl group, the pyridazinyl group, the pyrimidinyl group, and the pyrazinyl group may have one or more substituents selected from Group B}, a halogen atom, a hydroxy group, a cyano group, and CONR⁷²R⁷³,
R⁷² and R⁷³ are identical to or different from each other, and each represent a C1-C4 alkyl group optionally having one or more halogen atoms or a hydrogen atom,
Group B: a group consisting of a C1-C4 alkyl group optionally having one or more halogen atoms, a C1-C4 alkoxy group optionally having one or more halogen atoms, a C1-C4 alkylthio group optionally having one or more halogen atoms, a C1-C4 alkylsulfinyl group optionally having one or more halogen atoms, a C1-C4 alkylsulfonyl group optionally having one or more halogen atoms, a (C1-C4 alkoxy) carbonyl group wherein the C1-C4 alkoxy group optionally has one or more halogen atoms, a phenyl group optionally having one or more halogen atoms, a phenoxy group optionally having one or more halogen atoms, a cyano group, a nitro group, a carboxy group, a hydroxy group, a halogen atom, and CONR⁷⁴R⁷⁵;
R⁷⁴ and R⁷⁵ are identical to or different from each other, and each represent a C1-C4 alkyl group optionally having one or more halogen atoms or a hydrogen atom;
Group D: a group consisting of a hydroxy group and a halogen atom;
Group E: a group consisting of a C1-C4 alkoxy group optionally having one or more halogen atoms, a cyano group, a hydroxy group, and a halogen atom] or a salt thereof.

2. The compound or a salt thereof according to claim 1, wherein
R¹ and R² are identical to or different from each other, and each represent a C1-C6 chain hydrocarbon group optionally having one or more substituents selected from Group A, a C1-C3 alkoxy group optionally having one or more halogen atoms, a benzyl group optionally having one or more substituents selected from Group B, a C1-C3 alkylcarbonyl group optionally having one or more halogen atoms, a C1-C3 alkoxycarbonyl group optionally having one or more halogen atoms, a C1-C3 alkylsulfonyl group optionally having one or more halogen atoms, or a hydrogen atom; or
R¹ and R² are combined to form —(CR¹⁷R¹⁸)—(CR¹⁹R²⁰)_{b}—(CR²⁵R²⁶)—, or —(CR²⁷R²⁸)—(CR²⁹R³⁰)—Y—(CR³¹R³²)—(CR³³R³⁴)—:
Group A: a group consisting of a C3-C6 alicyclic hydrocarbon group optionally having one or more substituents selected from Group E, a C1-C4 alkoxy group optionally having one or more halogen atoms, a C1-C4 alkylcarbonyl group optionally having one or more halogen atoms, a C1-C4 alkoxycarbonyl group optionally having one or more halogen atoms, a C1-C4 alkylsulfonyl group optionally having one or more halogen atoms, a pyridyl group, a furyl group, a thienyl group, a pyridazinyl group, a pyrimidinyl group, a pyrazinyl group {the pyridyl group, the furyl group, the thienyl group, the pyridazinyl group, the pyrimidinyl group, and the pyrazinyl group may have one or more substituents selected from Group B}, a halogen atom, a hydroxy group, a cyano group, and CONR⁷²R⁷³.

3. The compound or a salt thereof according to claim 1, wherein
R¹ and R² are identical to or different from each other, and each represent a C1-C6 chain hydrocarbon group optionally having one or more substituents selected from Group A¹, a C1-C3 alkoxy group optionally having one or more halogen atoms, a benzyl group optionally having one or more substituents selected from Group F, or a hydrogen atom; or
R¹ and R² are combined to form —(CR¹⁷R¹⁸)—(CR¹⁹R²⁰)_{b}—(CR²⁵R²⁶)—, or —(CR²⁷R²⁸)—(CR²⁹R³⁰)—Y—(CR³¹R³²)—(CR³³R³⁴)—:
Group F: a group consisting of a C1-C4 alkyl group optionally having one or more halogen atoms, a C1-C4 alkoxy group optionally having one or more halogen atoms, a (C1-C4 alkoxy) carbonyl group wherein the C1-C4 alkoxy group optionally has one or more halogen atoms, a cyano group, a nitro group, a carboxy group, a hydroxy group, and a halogen atom.

4. The compound or a salt thereof according to claim 2, wherein
R¹ and R² are identical to or different from each other, and each represent a C1-C6 chain hydrocarbon group optionally having one or more substituents selected from Group A, a C1-C3 alkoxy group optionally having one or more halogen atoms, a benzyl group optionally having one or more substituents selected from Group F, or a hydrogen atom; or
R¹ and R² are combined to form —(CR¹⁷R¹⁸)—(CR¹⁹R²⁰)_{b}—(CR²⁵R²⁶)—, or —(CR²⁷R²⁸)—(CR²⁹R³⁰)—Y—(CR³¹R³²)—(CR³³R³⁴)—:
Group F: a group consisting of a C1-C4 alkyl group optionally having one or more halogen atoms, a C1-C4 alkoxy group optionally having one or more halogen atoms, a (C1-C4 alkoxy) carbonyl group wherein the C1-C4 alkoxy group optionally has one or more halogen atoms, a cyano group, a nitro group, a carboxy group, a hydroxy group, and a halogen atom.

5. The compound or a salt thereof according to claim 1, wherein
R¹², R¹³, R¹⁴, R¹⁵, and R¹⁶ are identical to or different from each other, and each represent a C1-C4 chain hydrocarbon group optionally having one or more substituents selected from Group E, a C1-C4 alkoxy group optionally having one or more substituents selected from Group E, a C3-C6 alicyclic hydrocarbon group optionally having one or more substituents selected from Group E, a cyano group, a formyl group, a carboxy group, a nitro group, a hydroxy group, a halogen atom, or a hydrogen atom.

6. The compound or a salt thereof according to claim 1, wherein
X represents an oxygen atom or a sulfur atom.

7. The compound or a salt thereof according to claim 1, wherein
R⁹ represents a C1-C3 chain hydrocarbon group optionally having one or more substituents selected from Group D or a hydrogen atom.

8. The compound or a salt thereof according to claim 1, wherein
R¹⁰ and R¹¹ each represent a hydrogen atom.

9. The compound or a salt thereof according to claim 1, wherein
p represents 0 or 1.

10. The compound or a salt thereof according to claim 1, wherein
p represents 0.

11. A composition for controlling a harmful arthropod comprising the compound or a salt thereof according to claim 1, and an inert carrier.

12. A composition comprising one or more ingredients selected from the group consisting of Group (a), Group (b), Group (c), and Group (d), and the compound or a salt thereof according to claim 1:
Group (a): a group consisting of insecticidal active ingredients, miticidal active ingredients, and nematicidal active ingredients;
Group (b): fungicidal active ingredients;
Group (c): plant growth modulating ingredients; and
Group (d): repellent ingredients.

13. A method for controlling a harmful arthropod, which comprises applying an effective amount of the compound or a salt thereof according to claim 1 to a harmful arthropod or a habitat where the harmful arthropod lives.

14. A seed or vegetative reproductive organ carrying an effective amount of the compound or a salt thereof according to claim 1.

15. A method for controlling a harmful arthropod, which comprises applying an effective amount of the composition according to claim 12 to a harmful arthropod or a habitat where the harmful arthropod lives.

16. A seed or vegetative reproductive organ carrying an effective amount of the composition according to claim 12.
